SEQUENCE LISTING (10 sequences)

SEQ ID NO: 1 - protein, 30 aa, Meleagris gallopavo
DGWLPPDCGPHNRSGGGGATAAPTGSRQVS

SEQ ID NO: 2 - protein, 35 aa, Meleagris gallopavo
CEGRFYGSQEQPQPPPLPQHQPILGNGRASKRKTS

SEQ ID NO: 3 - protein, 115 aa, Meleagris gallopavo
AGGQPAPLPGGFISTLGSPEHSPGGTWSDCNGGTRGGSESSLEERHSKTSRSESKMEREKNILATTRFYCTFLGNGDKAVFCTVLRIVKLFEDATCTCPHTHKLKMKWRFKQHQA

SEQ ID NO: 4 - protein, 483 aa, Meleagris Gallopavo
MGDGWLPPDCGPHNRSGGGGATAAPTGSRQVSAELLSQQWEAGMSLLMALVVLLIVAGNVLVIAAIGRTQRLQTLTNLFITSLACADLVMGLLVVPFGATLVVRGTWLWGSFLCECWTSLDVLCVTASIETLCVIAIDRYLAITSPFRYQSLMTRARAKVIICTVWAISALVSFLPIMMHWWRDEDPQALKCYQDPGCCDFVTNRAYAIASSIISFYIPLLIMIFVYLRVYREAKEQIRKIDRCEGRFYGSQEQPQPPPLPQHQPILGNGRASKRKTSRVMAMREHKALKTLGIIMGVFTLCWLPFFLVNIVNVFNRDLVPDWLFVFFNWLGYANSAFNPIIYCRSPDFRKAFKRLLCFPRKADRRLHAGGQPAPLPGGFISTLGSPEHSPGGTWSDCNGGTRGGSESSLEERHSKTSRSESKMEREKNILATTRFYCTFLGNGDKAVFCTVLRIVKLFEDATCTCPHTHKLKMKWRFKQHQA

SEQ ID NO: 5 - protein, 477 aa, Homo sapiens
MGAGVLVLGASEPGNLSSAAPLPDGAATAARLLVPASPPASLLPPASESPEPLSQQWTAGMGLLMALIVLLIVAGNVLVIVAIAKTPRLQTLTNLFIMSLASADLVMGLLVVPFGATIVVWGRWEYGSFFCELWTSVDVLCVTASIETLCVIALDRYLAITSPFRYQSLLTRARARGLVCTVWAISALVSFLPILMHWWRAESDEARRCYNDPKCCDFVTNRAYAIASSVVSFYVPLCIMAFVYLRVFREAQKQVKKIDSCERRFLGGPARPPSPSPSPVPAPAPPPGPPRPAAAAATAPLANGRAGKRRPSRLVALREQKALKTLGIIMGVFTLCWLPFFLANVVKAFHRELVPDRLFVFFNWLGYANSAFNPIIYCRSPDFRKAFQRLLCCARRAARRRHATHGDRPRASGCLARPGPPPSPGAASDDDDDDVVGATPPARLLEPWAGCNGGAAADSDSSLDEPCRPGFASESKV

SEQ ID NO: 6 - protein, 413 aa, Homo sapiens
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 7 - protein, 408 aa, Homo sapiens
MAPWPHENSSLAPWPDLPTLAPNTANTSGLPGVPWEAALAGALLALAVLATVGGNLLVIVAIAWTPRLQTMTNVFVTSLAAADLVMGLLVVPPAATLALTGHWPLGATGCELWTSVDVLCVTASIETLCALAVDRYLAVTNPLRYGALVTKRCARTAVVLVWVVSAAVSFAPIMSQWWRVGADAEAQRCHSNPRCCAFASNMPYVLLSSSVSFYLPLLVMLFVYARVFVVATRQLRLLRGELGRFPPEESPPAPSRSLAPAPVGTCAPPEGVPACGRRPARLLPLREHRALCTLGLIMGTFTLCWLPFFLANVLRALGGPSLVPGPAFLALNWLGYANSAFNPLIYCRSPDFRSAFRRLLCRCGRRLPPEPCAAARPALFPSGVPAARSSPAQPRLCQRLDGASWGVS

SEQ ID NO: 8 - protein, 313 aa, Meleagris gallopavo
MGAELLSQQWEAGMSLLMALVVLLIVAGNVLVIAAIGSTQRLQTLTNLFITSLACADLVVGLLVVPFGATLVVRGTWLWGSFLCELWTSLDVLCVTASIETLCVIAIDRYLAITSPFRYQSLMTRARAKVIICTVWAISALVSFLPIMMHWWRDEDPQALKCYQDPGCCDFVTNRAYAIASSIISFYIPLLIMIFVALRVYREAKEQIRKIDRASKRKRVMLMREHKALKTLGIIMGVFTLCWLPFFLVNIVNVFNRDLVPDWLFVAFNWLGYANSAMNPIIYCRSPDFRKAFKRLLAFPRKADRRLHHHHHH

SEQ ID NO: 9 - protein, 500 aa, Homo sapiens
DYKDDDAMGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYAEETCCDFFTNQAYAIASSIVSFYVPLVIMVFVYSRVFQEAKRQLNIFEMLRIDEGLRLKIYKDTEGYYTIGIGHLLTKSPSLNAAKSELDKAIGRNTNGVITKDEAEKLFNQDVDAAVRGILRNAKLKPVYDSLDAVRRAALINMVFQMGETGVAGFTNSLRMLQQKRWDEAAVNLAKSRWYNQTPNRAKRVITTFRTGTWDAYKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSG

SEQ ID NO: 10 - protein, 365 aa, Homo sapiens
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSG

(19) United States
(12) Patent Application Publication
(10) Pub. No.: US 2011/0112037 A1
(43) Pub. Date: May 12, 2011

(54) CRYSTAL STRUCTURE

(75) Inventors: Antony Johannes Warne, Cambridge (GB); Maria Josefa Serrano-Vega, Cambridge (GB); Rouslan Moukhametzianov, Cambridge (GB); Patricia C. Edwards, Cambridge (GB); Richard Henderson, Cambridge (GB); Andrew G.W. Leslie, Cambridge (GB); Christopher Gordon Tate, Cambridge (GB); Gebhard F.X. Schertler, Cambridge (GB)

(73) Assignee: Heptares Therapeutics Limited BioPark, Hertfordshire (GB)

(21) Appl. No.: 12/921,036

(22) PCT Filed: Mar. 5, 2008
(86) PCT No.: PCT/GB08/00740

§ 371 Date: Jan. 14, 2011

(51) Int. Cl. C07K 14/705; G01N 33/68; C07K 2/00; C07K 1/00; C07D 209/42; C07K 16/28; C07H 21/00; C07C 53/00; C12N 9/12; A61K 38/02; A61P 9/00; G06G 7/58

(52) U.S. Cl. 514/20.6; 436/86; 530/350; 530/300; 548/505; 530/389.1; 530/387.3; 536/23.1; 554/1; 435/194; 703/11

(57) ABSTRACT

The invention provides a method of predicting a three dimensional structural representation of a target protein of unknown structure, or part thereof, comprising: providing the coordinates of the turkey β1-AR structure listed in Table A, Table B, Table C or Table D, optionally varied by a root mean square deviation of residue backbone atoms of not more than 1.235 A, or selected coordinates thereof; and predicting the three-dimensional structural representation of the target protein, or part thereof, by modelling the structural representation on all or the selected coordinates of the turkey β1-AR. The invention also provides the use of the turkey β1-AR coordinates to select or design one or more binding partners of β1-AR.

The present invention relates to protein crystal structures and their use in identifying protein binding partners and in protein structure determination. In particular, it relates to the crystal structure of a ββ-adrenergic receptor (β1-AR) and uses thereof.

The listing or discussion of an apparently prior-published document in this specification should not necessarily be taken as an acknowledgement that the document is part of the state of the art or is common general knowledge.

G protein-coupled receptors (GPCRs) are a large family of integral membrane proteins that are ubiquitous in eukaryotes from yeast to man, which function as key intermediaries in the transduction of signals from the outside of the cell to the inside. Activating molecules (agonists), such as hormones and neurotransmitters, bind to the GPCRs at the cell surface and cause a conformational change at the cytoplasmic surface, resulting in the activation of G proteins and the resultant increase in intracellular messengers such as cAMP, Ca²⁺ and signalling lipids. The central role of GPCRs in signalling throughout the body makes them ideal targets for therapeutic agents and, in fact, about 30% of prescription drugs mediate their effects by binding specifically to GPCRs and it is thought that developing new specific compounds to inhibit or activate other GPCRs could represent a major route to the development of new drugs.

There are about 850 different GPCRs in the human body and they all share the characteristic of 7 transmembrane domains with their N terminus in the extracellular side of the plasma membrane. Analysis of their primary amino acid sequence has resulted in the definition of a number of subfamilies, the largest of which, Family A, includes the archetypal GPCR, rhodopsin. One of the subdivisions within Family A contains the aminergic receptors, which include, for example, serotonin, dopamine, acetylcholine and adrenergic receptors. The natural ligand for adrenergic receptors is either adrenaline, released into the blood from the adrenal glands, or noradrenaline, which is a neurotransmitter in the brain, but also acts peripherally. The adrenergic receptors are further divided into two groups, the α- and β-adrenergic receptors, originally classified depending on whether they caused contraction or relaxation of tissues. There are three β-adrenergic (β-AR) subtypes in humans, β1, β2 and β3 and they share 53% sequence identity, excluding the N- and C-termini and inner loop 3. There is a wealth of pharmacology associated with the βARs, because molecules that inhibit receptor signalling (antagonists) are capable of modulating the function of the heart and are commonly known as β-blockers. Non-selective β-blockers such as propranolol were used in treatment of hypertension or for cardioprotection after a heart attack (inhibition of the β1-AR), but more recently selective β1-antagonists are preferred since they have fewer side effects due to bronchial constriction (β2 effect). The development of β-blockers followed classical pharmacological characterisation of small molecules that inhibited signalling of βARs, which has resulted in a multitude of compounds that differentially effect the three different subtypes (Baker J G (2005) British Journal Pharmanol. Vol 144, pp 317-322). However, it has been unclear what determines the specificity of drug binding to the specific subtypes; elucidation of this mechanism will allow the development of more subtype-specific β-blockers and hence reduce side-effects for various patient groups.

Two independently determined structures of the β2-adrenergic receptor (β2-AR) that both contained bound antagonist (specifically, a partial inverse agonist) carazolol have recently been published (Rasmussen et al 2007; Cherezov et al 2007). The structures define the overall architecture of the protein and provide a description of the ligand binding region and how amino acid residues contribute to the specificity of the ligand bound. However, the structures also raise many questions of how different βARs bind the same ligand with different affinities. For example, the human β1 and β2 receptors are 69% identical within their transmembrane regions, but if only the residues that were predicted to surround the ligand binding region in the β2 structure are considered, then the receptors are apparently identical. Despite these similarities, compounds such as CGP20712A bind 500 times more strongly to the β1 receptor than to the β2 receptor, whilst ICI 118551 shows a 550 fold specificity for the β2 receptor over β1 (Baker J G (2005) British Journal Pharmacol. Vol 144, pp 317-322). Ideally, the structures of both the β1 and β2 receptors need to be compared to elucidate the mechanism behind drug discrimination.

We have now crystallised and determined the first structure of a β1-AR, the turkey β1-AR, in complex with the antagonist cyanopindolol using X-ray crystallography. Crystals of a stabilised mutant turkey β1-AR receptor (β1-AR-m23) were crystallised in a variety of detergents and conditions, giving rise to two predominant forms with either C2 or P1 geometry. In both space groups there were four molecules per unit cell (molecules A-D). The structure was solved to a resolution of 2.7 Å by molecular replacement using the coordinates of the β2-AR (Cherezov et al, 2007). The atomic coordinates of molecules A-D are provided in Tables A-D respectively.

The coordinates of the β1-AR can be utilised and manipulated in many different ways with wide ranging applications including the fitting of binding partners, homology modelling and structure solution, analysis of ligand interactions and drug discovery.

Accordingly, a first aspect of the invention provides a method of predicting a three dimensional structural representation of a target protein of unknown structure, or part thereof, comprising:
providing the coordinates of the turkey β1-AR structure listed in Table A, Table B, Table C or Table D, optionally varied by a root mean square deviation of residue backbone atoms of not more than 1.235 Å, or selected coordinates thereof; and
predicting the three-dimensional structural representation of the target protein, or part thereof, by modelling the structural representation on all or the selected coordinates of the turkey β1-AR.

By a ‘three dimensional structural representation’ we include a computer generated representation or a physical representation. Typically, in all aspects of the invention which feature a structural representation, the representation is computer generated. Computer representations can be generated or displayed by commercially available software programs. Examples of software programs include but are not limited to QUANTA (Accelrys. COPYRIGHT. 2001, 2002), O (Jones et al., Acta Crystallogr. A47, pp. 110-119 (1991)) and RIBBONS (Carson, J. Appl. Crystallogr., 24, pp. 9589-961 (1991)), which are incorporated herein by reference. Examples of representations include any of a wire-frame model, a chicken-wire model, a ball-and-stick model, a space-filling model, a stick model, a ribbon model, a snake model, an arrow and cylinder model, an electron density map or a molecular surface model. Certain software programs may also imbue these three dimensional representations with physico-chemical attributes which are known from the chemical composition of the molecule, such as residue charge, hydrophobicity, torsional and rotational degrees of freedom for the residue or segment, etc. Examples of software programs for calculating chemical energies are described below.

Typically, the coordinates of the turkey β1-AR structure used in the invention are those listed in Table A, Table B, Table C or Table D. Preferably the coordinates used are of molecule B in Table B. However, it is appreciated that it is not necessary to have recourse to the original coordinates listed in Table A, Table B, Table C or Table D and that any equivalent geometric representation derived from or obtained by reference to the original coordinates may be used.

Thus, for the avoidance of doubt, by ‘the coordinates of the turkey β1-AR structure listed in Table A, Table B, Table C or Table D’, we include any equivalent representation wherein the original coordinates have been reparameterised in some way. For example, the coordinates in Table A, Table B, Table C or Table D may undergo any mathematical transformation known in the art, such as a geometric transformation, and the resulting transformed coordinates can be used. For example, the coordinates of Table A, Table B, Table C or Table D may be transposed to a different origin and/or axes or may be rotated about an axis. Furthermore, it is possible to use the coordinates to calculate the psi and phi backbone torsion angles (as displayed on a Ramachandran plot) and the chi sidechain torsion angles for each residue in the protein. These angles together with the corresponding bond lengths, enable the construction of a geometric representation of the protein which may be used based on the parameters of psi, phi and chi angles and bond lengths. Thus while the coordinates used are typically those in Table A, Table B, Table C or Table D, the inventors recognise that any equivalent geometric representation of the turkey β1-AR structure, based on the coordinates listed in Table A, Table B, Table C or Table D, may be used.

Additionally, it is appreciated that changing the number and/or positions of the water molecules and/or ligand molecule of the Tables does not generally affect the usefulness of the coordinates in the aspects of the invention. Thus, it is also within the scope of the invention if the number and/or positions of water molecules and/or ligand molecules of the coordinates of Table A, Table B, Table C or Table D is varied.

It will be appreciated that in all aspects of the invention which utilise the coordinates of the turkey β1-AR, it is not necessary to utilise all the coordinates of Table A, Table B, Table C or Table D, but merely a portion of them, e.g. a set of coordinates representing atoms of particular interest in relation to a particular use. Such a portion of coordinates is referred to herein as ‘selected coordinates’.

By ‘selected coordinates’, we include at least 5, 10 or 20 non-hydrogen protein atoms of the turkey β1-AR structure, more preferably at least 50, 100, 200, 300, 400, 500, 600, 700, 800 or 900 atoms and even more preferably at least 1000, 1100, 1200, 1300, 1400, 1500, 1600, 1700, 1800, 1900, 2000, 2100 or 2200 non-hydrogen atoms. Preferably the selected coordinates pertain to at least 0.5, 10, 20 or 30 different amino acid residues (i.e. at least one atom from 5, 10, 20 or 30 different residues may be present), more preferably at least 40, 50, 60, 70, 80 or 90 residues, and even more preferably at least 100, 150, 200, 250 or 300 residues. Optionally, the selected coordinates may include one or more ligand atoms and/or water atoms and/or sodium atoms as set out in Table A, Table B, Table C or Table D. Alternatively, the selected coordinates may exclude one or more water atoms or sodium atoms or may exclude one or more atoms of the ligand.

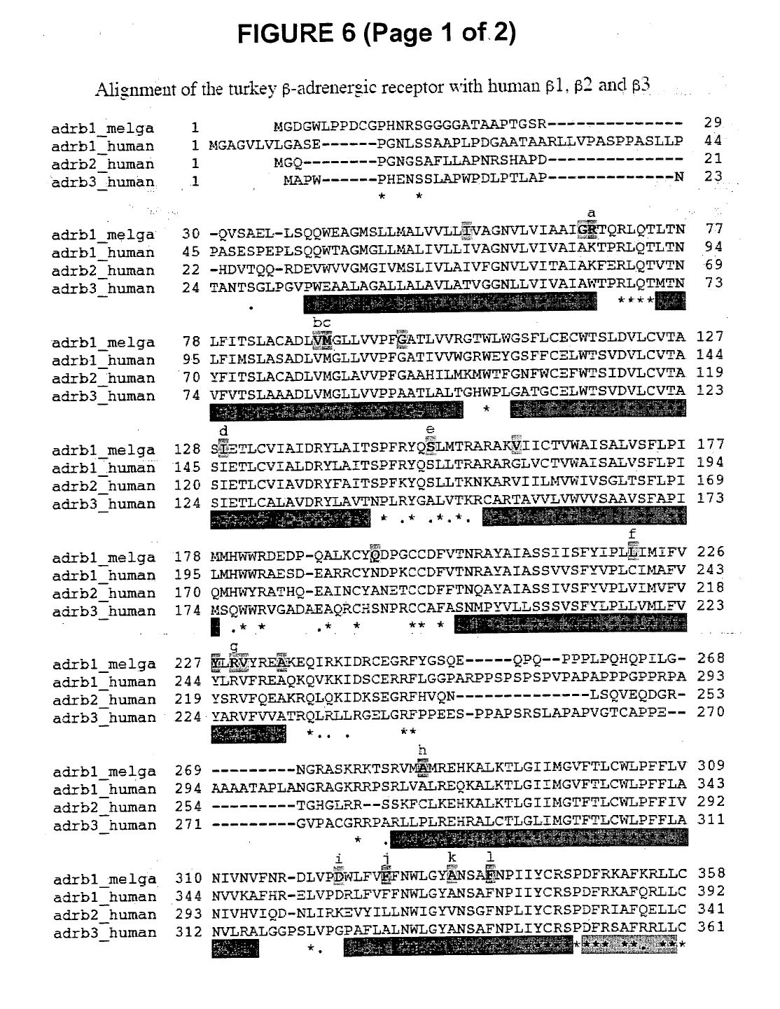

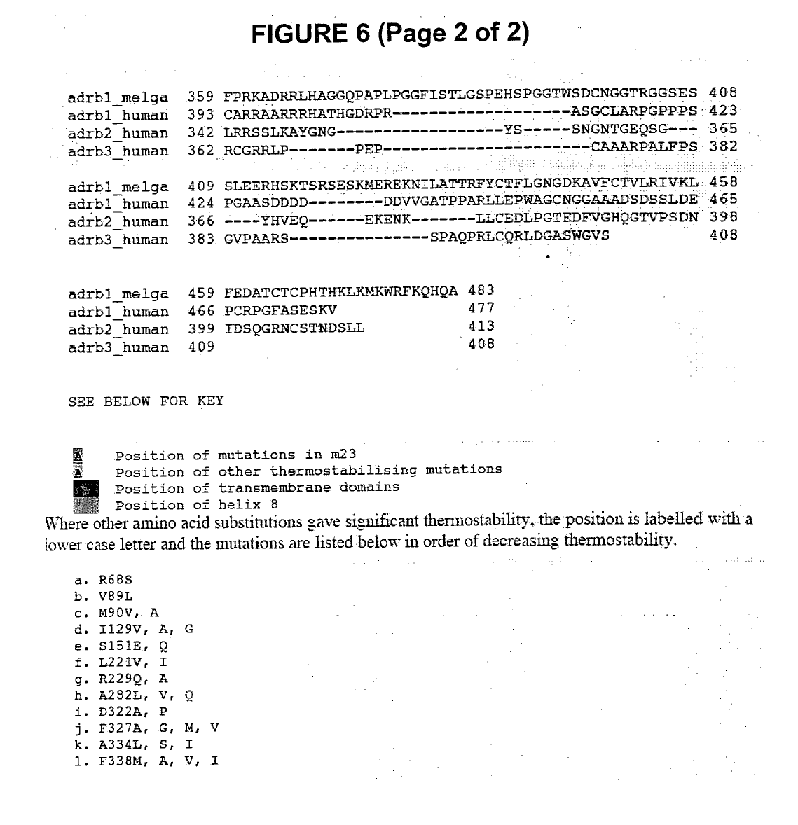

In one example, the selected coordinates may comprise atoms of one or more amino acid residues that contribute to the main chain or side chain atoms of a binding region of the turkey β1-AR. For example, amino acid residues contributing to the ligand binding site include amino acid residues 117, 118, 121, 122, 125, 201, 203, 207, 211, 215, 306, 307, 310 and 329, according to the numbering of turkey β1-AR as set out in FIG. 6, all of which make direct contact to the ligand cyanopindolol ligand. Thus the selected coordinates may comprise one or more atoms from any one or more of amino acid residues 117, 118, 121, 122, 125, 201, 203, 207, 211, 215, 306, 307, 310 and 329, according to the numbering of turkey β1-AR as set out in FIG. 6. Typically, coordinates of all of the atoms of the side chain are selected.

In another example, the selected coordinates may comprise atoms which coordinate a sodium ion. For example, an interesting observation of the β1-AR structure is the presence of a well coordinated sodium ion at the C-terminus of the short extracellular loop-1 (EL1) helix in a location often found for positive ions or ligands at the negative end of the α-helix dipole. The sodium ion is coordinated by the carbonyl groups in the peptide backbone from residues Cys 192, Asp 195 and Cys 198 and one water molecule. Thus, the selected coordinates may comprise one or more (for example all atoms of the side chain) atoms of any one or more of these residues and the water molecule which coordinates the sodium ion.

In a further example, the selected coordinates may comprise atoms of one or more amino acids in cytoplasmic loop-2 (CL2) which mediates coupling of the GPCR to G proteins when in the activated state. The cytoplasmic loop structure of CL2 in β1-AR is significantly different from that in β2-AR despite the amino acid sequence of CL2 being almost identical in the β-AR family. Specifically, CL2 in β1-AR is a well-structured short α-helix, whereas in the β2 structures CL2 is unstructured. Thus, the selected coordinates may comprise atoms of one or more of amino acid residues Ser 145, Pro 146, Phe 147, Arg 148, Tyr 149, Gln 150, Ser 151, Leu 152, Met 153 and Thr 154.

In another example, the selected coordinates may comprise atoms of one or more amino acids which define the conserved DRY motif in helix 3 of GPCRs. The DRY motif has been implicated both in G protein coupling and in the regulation of receptor activation (Rovati et al 2007, Mol Pharmacol 71(4): 959). Thus, the selected coordinates may comprise atoms of one or more of amino acid residues Asp 138, Arg 139 and Tyr 140.

In a further example, the selected coordinates may comprise atoms of one or more of the amino acids that define the binding region and are highly conserved in β1-ARs but not in β2-ARs. For example, residues Val 172 and Phe 325 are highly conserved in the β1 receptor but not in the β2 receptor whereas equivalent residues Thr 164 and Tyr 308 are highly conserved in the β2 receptor but not in the β1 receptor. Therefore, these residues are believed to have a profound effect upon ligand binding and selectivity. Thus, the selected coordinates may comprise atoms of Val 172 and/or Phe 325.

In yet a further example, the selected coordinates may comprise atoms of one or more of the amino acids in β1-AR which have been shown to be important in β1 versus β2 selectivity for particular ligands. For example amino residues Leu 110, Thr 117 and Phe 359 in β1-AR have been demonstrated to be important for the β1 selectivity of ligand RO363 (Sugimoto et al, 2002). Thus, the selected coordinates may comprise atoms of one or more of amino acids Leu 110, Thr 117 and Phe 359.

In another example, the selected coordinates may comprise atoms of an amino acid residue, mutation of which is a known polymorphism in the human β1AR family. For example, the human β1-AR mutation R389G corresponds to turkey β1-AR Arg 355 in C-terminal helix 8 and has a marked effect on in vitro function. Thus, the selected coordinates may comprise atoms of amino acid Arg 355.

It is appreciated that the selected coordinates may comprise any atoms of particular interest including atoms mentioned in any one or more of the above examples.

Preferably, the selected coordinates include at least 2% or 5% C-α atoms, and more preferably at least 10% C-α atoms. Alternatively or additionally, the selected coordinates include at least 10% and more preferably at least 20% or 30% backbone atoms selected from any combination of the nitrogen, C-α, carbonyl C and carbonyl oxygen atoms.

It is appreciated that the coordinates of the turkey β1-AR used in the invention may be optionally varied and a subset of the coordinates or the varied coordinates may be selected (and constitute selected coordinates). Indeed, such variation may be necessary in various aspects of the invention, for example in the modelling of protein structures and in the fitting of various binding partners to the β1-AR structure.

Protein structure variability and similarity is routinely expressed and measured by the root mean square deviation (rmsd), which measures the difference in positioning in space between two sets of atoms. The rmsd measures distance between equivalent atoms after their optimal superposition. The rmsd can be calculated over all atoms, over residue backbone atoms (i.e. the nitrogen-carbon-carbon backbone atoms of the protein amino acid residues), main chain atoms only (i.e. the nitrogen-carbon-oxygen-carbon backbone atoms of the protein amino acid residues), side chain atoms only or more usually over C-α atoms only.

The least-squares algorithms used to calculate rmsd are well known in the art and include those described by Rossman and Argos (J Biol Chem, (1975) 250:7525), Kabsch (Acta Cryst (1976) A92:922; Acta Cryst (1978) A34:827-828), Hendrickson (Acta Cryst (1979) A35: 158), McLachan (J Mol Biol (1979) 128:49) and Kearsley (Acta Cryst (1989) A45:208). Both algorithms based on iteration in which one molecule is moved relative to the other, such as that described by Ferro and Hermans (Acta Cryst (1977) A33:345-347), and algorithms which locate the best fit directly (e.g. Kabsch's methods) may be used. Methods of comparing proteins structures are also discussed in Methods of Enzymology, vol 115: 397-420.

Typically, rmsd values are calculated using coordinate fitting computer programs and any suitable computer program known in the art may be used, for example MNYFIT (part of a collection of programs called COMPOSER, Sutcliffe et al (1987) Protein Eng 1:377-384). Other programs also include LSQMAN (Kleywegt & Jones (1994) A super position, CCP4/ESF-EACBM, Newsletter on Protein Crystallography, 31: 9-14), LSQKAB (Collaborative Computational Project 4. The CCP4 Suite: Programs for Protein Crystallography, Acta Cryst (1994) D50:760-763), QUANTA (Jones et al, Acta Cryst (1991) A47:110-119 and commercially available from Accelrys, San Diego, Calif.), Insight (Commercially available from Accelrys, San Diego, Calif.), Sybyl® (commercially available from Tripos, Inc., St Louis) and O (Jones et al., Acta Cryst (1991) A47:110-119).

In, for example, the programs LSQKAB and O, the user can define the residues in the two proteins that are to be paired for the purpose of the calculation. Alternatively, the pairing of residues can be determined by generating a sequence alignment of the two proteins as is well known in the art. The atomic coordinates can then be superimposed according to this alignment and an rmsd value calculated. The program Sequoia (Bruns et al (1999) J Mol Biol 288(3):427-439) performs the alignment of homologous protein sequences, and the superposition of homologous protein atomic coordinates. Once aligned, the rmsd can be calculated using programs detailed above. When the sequences are identical or highly similar, the structural alignment of proteins can be done manually or automatically as outlined above. Another approach would be to generate a superposition of protein atomic coordinates without considering the sequence.

We have conducted an rmsd analysis of residue backbone atoms (i.e. the nitrogen-carbon-carbon backbone atoms of the protein) between the β1-AR (molecule B) and the β2-AR (Cherezov et al., 2007) using a LSQMAN script as shown in part B of Example 3. Similar scripts can be used to calculate rmsd values for any other selected coordinates. Rmsd values have been calculated on residue backbone atoms in the complete structure (1.235 Å), on residue backbone atoms used in aligning helices 2-6, on residue backbone atoms within the individual helices and on residue backbone atoms within the individual loop regions. Thus in an embodiment, where the coordinates or selected coordinates used in the invention are optionally varied within a particular structural region of the turkey β1-AR (e.g. helix 3 or just within the helices), they are optionally varied within an rmsd of residue backbone atoms of not more than the value corresponding to that structural region provided in part B of Example 3. For example, if the coordinates or selected coordinates are optionally varied within helix 3, they are optionally varied within an rmsd of residue backbone atoms of not more than 0.304 Å (such as not more than 0.3 Å or 0.2 Å or 0.1 Å) and if the coordinates or selected coordinates are optionally varied within extracellular loop 2, they are optionally varied within an rmsd of residue backbone atoms of not more than 0.836 Å (such as not more than 0.8 Å or 0.7 Å or 0.6 Å or 0.5 Å or 0.4 Å or 0.3 Å or 0.2 Å or 0.1 Å). By the helices and loop regions of the turkey β1-AR we mean the following:

### Helix 1 Residues 47-67

### Helix 2 Residues 77-98

### Helix 3 Residues 117-142

### Helix 4 Residues 156-173

### Helix 5 Residues 208-237

### Helix 6 Residues 286-310

### Helix 7 Residues 320-340

### Helix 8 Residues 341-358

### CL1 Residues 68-76

### EL1 Residues 99-116

### CL2 Residues 143-155

### EL2 Residues 174-207

### EL3 Residues 311-319

However, it will be appreciated that there are different criteria for which residues are considered to be in a helical conformation depending on phi and psi angles. Moreover, when comparing the turkey β1-AR to other structures, some residues may be missing in one or other of the structures and some residues may be considered helical in one structure but not the other. Therefore the limits above are not to be construed as absolute, but rather may vary according to the criteria used. Nevertheless, for the purposes of the comparisons set out below, we have used the above-mentioned definitions of helices and loops.

Thus in one embodiment, the coordinates or selected coordinates of Table A, Table B, Table C or Table D may be optionally varied within an rmsd of residue backbone atoms (i.e. the nitrogen-carbon-carbon backbone atoms of the protein) of not more than 1.235 Å. Preferably, the coordinates or selected coordinates are varied within an rmsd of residue backbone atoms of not more than 1.2 Å, 1.1 Å, 1.0 Å, 0.9 Å or 0.8 Å and more preferably not more than 0.7 Å, 0.6 Å, 0.5 Å, 0.4 Å, 0.3 Å, 0.2 Å or 0.1 Å.

Conducting an rmsd analysis of residue backbone atoms between β1-AR (molecule A; where N-terminal 50 residues of Helix 1 are omitted) and β2-AR (Cherezov et al, 2007) gave an rmsd value of 1.25 Å. Thus in one embodiment, the coordinates or selected coordinates of Table A, Table B, Table C or Table D may be optionally varied within an rmsd of residue backbone atoms of not more than 1.25 Å. Preferably, the coordinates or selected coordinates are varied within an rmsd of residue backbone atoms of not more than 1.2 Å, 1.1 Å, 1.0 Å, 0.9 Å or 0.8 Å and more preferably not more than 0.7 Å, 0.6 Å, 0.5 Å, 0.4 Å, 0.3 Å, 0.2 Å or 0.1 Å.

It is appreciated that rmsd can also be calculated over C-α atoms and side chain atoms.

For example, we aligned β1-AR (molecule B) with β2-AR (Cherezov et al, 2007) over the residues in helices 2-6, and a rmsd analysis of residue C-α atoms gave a value of 0.399 Å. The same analysis using β1-AR (molecule A) in the alignment gave a value of 0.401 Å. Thus, in one embodiment, the coordinates or selected coordinates are optionally varied within an rmsd of residue C-α atoms in helices 2-6 of not more than 0.40 Å. Preferably, the coordinates or selected coordinates are varied within an rmsd of residue C-α atoms in helices 2-6 of not more than 0.35 Å, 0.30 Å or 0.25 Å and more preferably not more than 0.2 Å, 0.15 Å or 0.10 Å.

We have conducted an rmsd analysis of residue C-α atoms and residue side chain atoms between β1-AR (molecule B) and β2-AR (Cherezov et al, 2007) within the active site (i.e. residues 117, 118, 121, 122, 125, 201, 203, 207, 211, 215, 306, 307, 310 and 329) as shown in Example 3. The rmsd value for residue C-α atoms is 0.38 Å and for side chain atoms is 0.59 Å. Thus in an embodiment, where the coordinates or selected coordinates used in the invention are optionally varied within the active site, they are varied within an rmsd of C-α atoms of not more than 0.38 Å (such as not more than 0.3 Å or 0.2 Å or 0.1 Å) and/or within an rmsd of side chain atoms of not more than 0.59 Å (such as not more than 0.5 Å or 0.4 Å or 0.3 Å or 0.2 Å or 0.1 Å).

We have conducted an rmsd analysis of residue C-α atoms and residue side chain atoms between β1-AR (molecule B) and β2-AR (Cherezov et al, 2007) within the Na ion coordination site (i.e. residues Cys 192, Asp 195 and Cys 198). The rmsd value for residue C-α atoms is 1.03 Å and for side chain atoms is 1.09 Å. Thus in an embodiment, where the coordinates or selected coordinates used in the invention are optionally varied within the Na ion coordination site, they are varied within an rmsd of C-α atoms of not more than 1.03 Å (such as not more than 1 Å or 0.9 Å or 0.8 Å or 0.7 Å or 0.6 Å or 0.5 Å or 0.4 Å or 0.3 Å or 0.2 Å or 0.1 Å) and/or within an rmsd of side chain atoms of not more than 1.09 Å (such as not more than 1 Å or 0.9 Å or 0.8 Å or 0.7 Å or 0.6 Å or 0.5 Å or 0.4 Å or 0.3 Å or 0.2 Å or 0.1 Å).

We have conducted an rmsd analysis of residue C-α atoms and residue side chain atoms between β1-AR (molecule B) and 62-AR (Cherezov et al, 2007) within the CL2 (i.e. residues 145-154). The rmsd value for residue C-α atoms is 5.66 Å and for side chain atoms is 6.88 Å. Thus in an embodiment, where the coordinates or selected coordinates used in the invention are optionally varied within the CL2, they are varied within an rmsd of C-α atoms of not more than 5.66 Å (such as not more than 5.5 Å or 5 Å or 4.5 Å or 4 Å or 3.5 Å or 3 Å or 2.5 Å or 2 Å or 1.5 Å or 1 Å or 0.5 Å) and/or within an rmsd of side chain atoms of not more than 6.88 Å (such as not more than 6.5 Å or 6 Å or 5.5 Å or 5 Å or 4.5 Å or 4 Å or 3.5 Å or 3 Å or 2.5 Å or 2 Å or 1.5 Å or 1 Å or 0.5 Å).

We have conducted an rmsd analysis of residue C-α atoms and residue side chain atoms between β1-AR (molecule B) and 62-AR (Cherezov et al, 2007) within the DRY motif (i.e. residues 138-140). The rmsd value for residue C-α atoms is 0.31 Å and for side chain atoms is 0.48 Å. Thus in an embodiment, where the coordinates or selected coordinates used in the invention are optionally varied within the DRY motif, they are varied within an rmsd of C-α atoms of not more than 0.31 Å (such as not more than 0.3 Å or 0.2 Å or 0.1 Å) and/or within an rmsd of side chain atoms of not more than 0.48 Å (such as not more than 0.4 Å or 0.3 Å or 0.2 Å or 0.1 Å).

We have conducted an rmsd analysis of residue backbone atoms and residue side chain atoms between β1-AR (molecule B) and 62-AR (Cherezov et al, 2007) within the residues Val 172 and Phe 325 which are believed to have a profound effect upon ligand binding and specificity. The rmsd value for residue backbone atoms is 0.72 Å and for side chain atoms is 1.99 Å. Thus in an embodiment, where the coordinates or selected coordinates used in the invention are optionally varied within the residues Val 172 and Phe 325, they are varied within an rmsd of residue backbone atoms of not more than 0.72 Å (such as not more than 0.7 Å or 0.6 Å or 0.5 Å or 0.4 Å or 0.3 Å or 0.2 Å or 0.1 Å) and/or within an rmsd of side chain atoms of not more than 1.99 Å (such as not more than 1.9 Å or 1.7 Å or 1.5 Å or 1.3 Å or 1.1 Å or 0.9 Å or 0.7 Å or 0.5 Å or 0.3 Å or 0.1 Å).

We have conducted an rmsd analysis of residue C-α atoms and residue side chain atoms between β1-AR (molecule B) and β2-AR (Cherezov et al, 2007) within the residues Leu 110, Thr 117 and Phe 359 which are thought to be important in ligand specificity. The rmsd value for residue C-α atoms is 0.94 Å and for side chain atoms is 0.92 Å. Thus in an embodiment, where the coordinates or selected coordinates used in the invention are optionally varied within the residues Leu 110, Thr 117 and Phe 359, they are varied within an rmsd of C-α atoms of not more than 0.94 Å (such as not more than 0.9 Å or 0.8 Å or 0.7 Å or 0.6 Å or 0.5 Å or 0.4 Å or 0.3 Å or 0.2 Å or 0.1 Å) and/or within an rmsd of side chain atoms of not more than 0.92 Å (such as not more than 0.9 Å or 0.8 Å or 0.7 Å or 0.6 Å or 0.5 Å or 0.4 Å or 0.3 Å or 0.2 Å or 0.1 Å).

In this aspect of the invention, the coordinates of the turkey β1-AR structure are used to predict a three dimensional representation of a target protein of unknown structure, or part thereof, by modelling. By “modelling”, we mean the prediction of structures using computer-assisted or other de novo prediction of structure, based upon manipulation of the coordinate data from Table A, Table B, Table C or Table D or selected coordinates thereof.

The target protein may be any protein that shares sufficient sequence identity to the turkey β1-AR such that its structure can be modelled by using the turkey β1-AR coordinates of Table A, Table B, Table C or Table D. It will be appreciated that if a structural representation of only a part of the target protein is being modelled, for example a particular domain, the target protein only has to share sufficient sequence identity to the turkey β1-AR over that part.

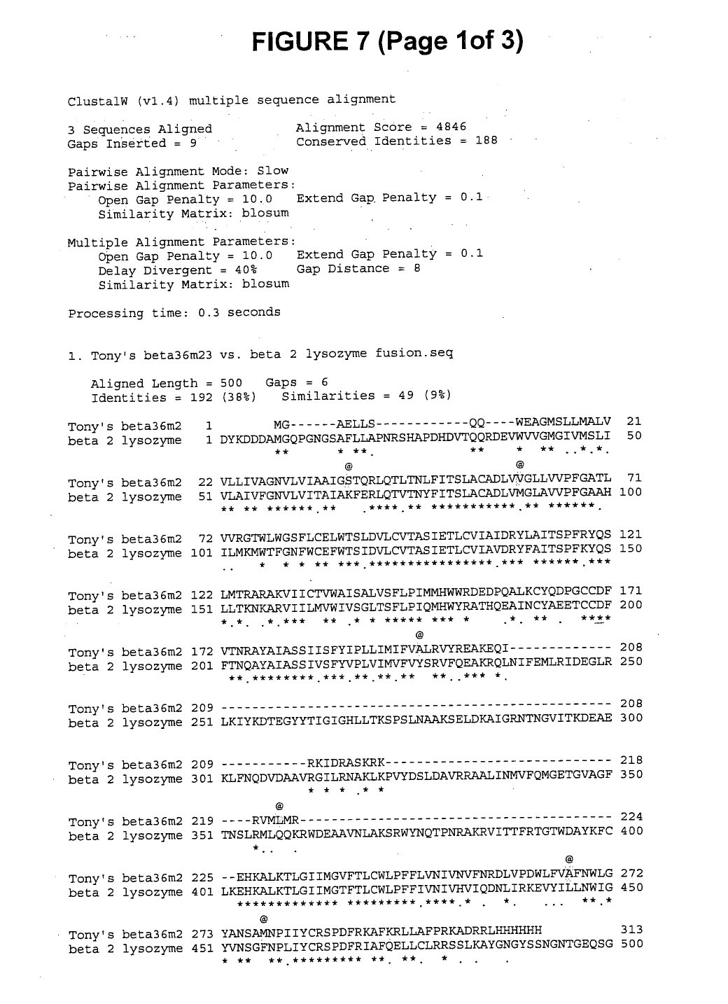

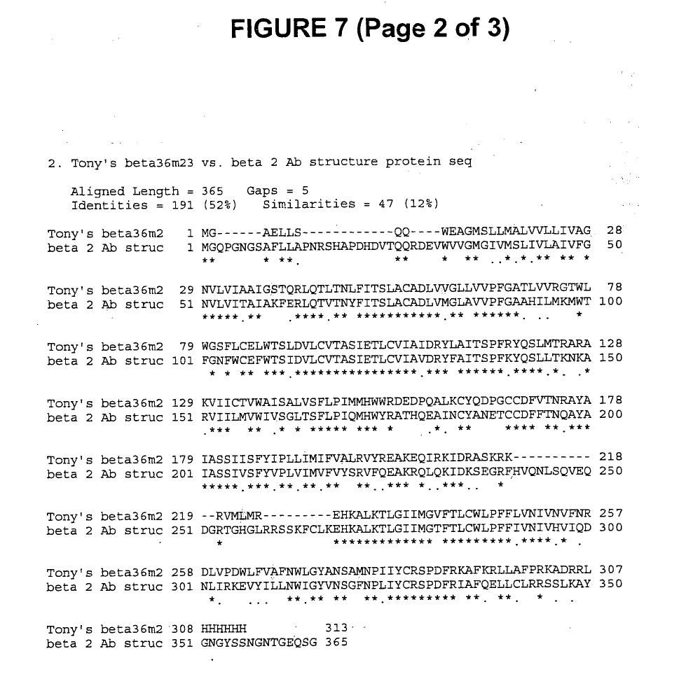

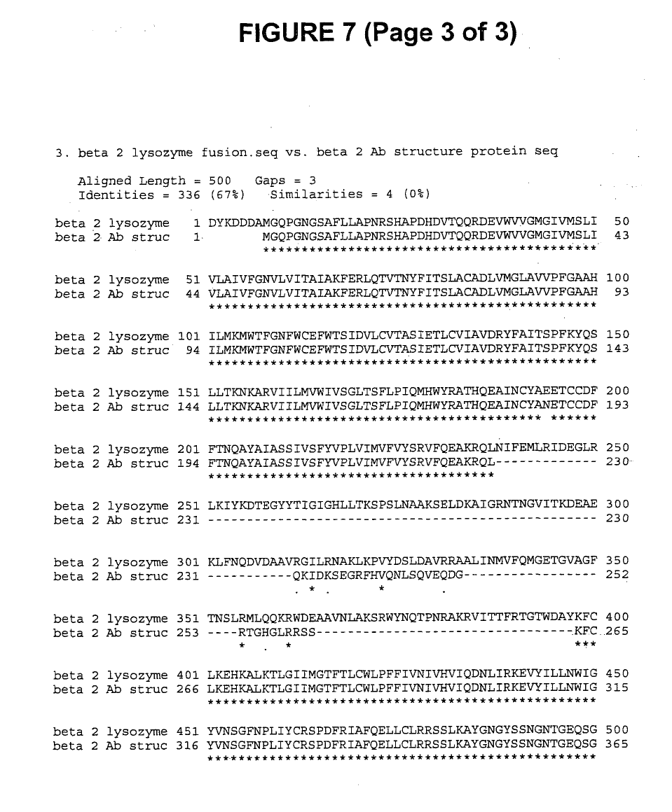

It has been shown for soluble protein domains that their three dimensional structure is broadly conserved above 20% amino acid sequence identity and well conserved above 30% identity, with the level of structural conservation increasing as amino acid sequence identity increases up to 100% (Ginalski, K. Curr Op Struc Biol (2006) 16, 172-177). Thus, it is preferred if the target protein, or part thereof, shares at least 20% amino acid sequence identity with turkey β1-AR sequence provided in FIG. 7, and more preferably at least 30%, 40%, 50%, 60%, 70%, 80% or 90% sequence identity, and yet more preferably at least 95% or 99% sequence identity.

It will be appreciated therefore that the target protein may be a turkey β1-AR analogue or homologue.

Analogues are defined as proteins with similar three-dimensional structures and/or functions with little evidence of a common ancestor at a sequence level.

Homologues are proteins with evidence of a common ancestor, i.e. likely to be the result of evolutionary divergence and are divided into remote, medium and close sub-divisions based on the degree (usually expressed as a percentage) of sequence identity.

By a turkey β1-AR homologue, we include a protein with at least 20%, 25%, 30%, 35%, 40%, 45% or at least 50% amino acid sequence identity with the sequence of turkey β1-AR provided in FIG. 7, preferably at least 55%, 60%, 65%, 70%, 75% or 80% amino acid sequence identity and more preferably 85%, 90%, 95% or 99% amino acid sequence identity. This includes polymorphic forms of β1-ARs, e.g. mutants and β1-ARs from other species as well as other β-adrenergic receptors such as β2-ARs and β3-ARs. For example, the turkey β1-AR shares 82%, 65% and 58% amino acid sequence identity with human β1-AR, human β2-AR and human β3-AR respectively (when excluding CL3 and N- and C-termini). Thus a turkey β1-AR homologue would include a human β1-AR, a human 32-AR and a human β3-AR.

Sequence identity may be measured by the use of algorithms such as BLAST or PSI-BLAST (Altschul et al, NAR (1997), 25, 3389-3402) or methods based on Hidden Markov Models (Eddy S et al, J Comput Biol (1995) Spring 2 (1) 9-23). Typically, the percent sequence identity between two polypeptides may be determined using any suitable computer program, for example the GAP program of the University of Wisconsin Genetic Computing Group and it will be appreciated that percent identity is calculated in relation to polypeptides whose sequence has been aligned optimally. The alignment may alternatively be carried out using the Clustal W program (Thompson et al., 1994). The parameters used may be as follows: Fast pairwise alignment parameters: K-tuple(word) size; 1, window size; 5, gap penalty; 3, number of top diagonals; 5. Scoring method: x percent. Multiple alignment parameters: gap open penalty; 10, gap extension penalty; 0.05. Scoring matrix: BLOSUM.

In one embodiment the target protein is an integral membrane protein. By “integral membrane protein” we mean a protein that is permanently integrated into the membrane and can only be removed using detergents, non-polar solvents or denaturing agents that physically disrupt the lipid bilayer. Examples include receptors such as GPCRs, the T-cell receptor complex and growth factor, receptors; transmembrane ion channels such as ligand-gated and voltage gated channels; transmembrane transporters such as neurotransmitter transporters; enzymes; carrier proteins; and ion pumps.

The amino acid sequences (and the nucleotide sequences of the cDNAs which encode them) of many membrane proteins are readily available, for example by reference to GenBank. For example, Foord et al supra gives the human gene symbols and human, mouse and rat gene IDs from Entrez Gene (http://www.ncbi.nlm.nih.gov/entrez) for GPCRs. It should be noted, also, that because the sequence of the human genome is substantially complete, the amino acid sequences of human membrane proteins can be deduced therefrom.

In a preferred embodiment, the target protein is a GPCR.

Suitable GPCRs include, but are not limited to β-adrenergic receptors, adenosine receptors, in particular the adenosine A₂ₐ receptor, neurotensin receptors (NTR) and muscarinic receptors. Other suitable GPCRs are well known in the art and include those listed in Hopkins & Groom supra. In addition, the International Union of Pharmacology produce a list of GPCRs (Foord et al (2005) Pharmacol. Rev. 57, 279-288, incorporated herein by reference and this list is periodically updated at http://www.iuphar-db.org/GPCR/ReceptorFamiliesForward). It will be noted that GPCRs are divided into different classes, principally based on their amino acid sequence similarities. They are also divided into families by reference to the natural ligands to which they bind. All GPCRs are included in the scope of the invention and their structure may be modelled by using the coordinates of the turkey β1-AR.

Although the target protein may be derived from any source, it is particularly preferred if it is from a eukaryotic source. It is particularly preferred if it is derived from a vertebrate source such as a mammal or a bird. It is particularly preferred if the target protein is derived from rat, mouse, rabbit or dog or non-human primate or man, or from chicken or turkey.

Typically, modelling a structural representation of a target is done by homology modelling whereby homologous regions between the turkey β1-AR and the target protein are matched and the coordinate data of the turkey β1-AR used to predict a structural representation of the target protein.

The term “homologous regions” describes amino acid residues in two sequences that are identical or have similar (e.g. aliphatic, aromatic, polar, negatively charged, or positively charged) side-chain chemical groups. Identical and similar residues in homologous regions are sometimes described as being respectively “invariant” and “conserved” by those skilled in the art.

Typically, the method involves comparing the amino acid sequences of turkey β1-AR with a target protein by aligning the amino acid sequences. Amino acids in the sequences are then compared and groups of amino acids that are homologous (conveniently referred to as “corresponding regions”) are grouped together. This method detects conserved regions of the polypeptides and accounts for amino acid insertions or deletions.

Homology between amino acid sequences can be determined using commercially available algorithms known in the art. For example, the programs BLAST, gapped BLAST, BLASTN, PSI-BLAST, BLAST 2 and WU-BLAST (provided by the National Center for Biotechnology Information) can be used to align homologous regions of two, or more, amino acid sequences. These may be used with default parameters to determine the degree of homology between the amino acid sequence of the turkey β1-AR and other target proteins which are to be modelled.

Preferred for use according to the present invention is the WU-BLAST (Washington University BLAST) version 2.0 software. WU-BLAST version 2.0 executable programs for several UNIX platforms can be downloaded from ftp://blast. wustl. edu/blast/executables. This program is based on WU-BLAST version 1.4, which in turn is based on the public domain NCBI-BLAST version 1.4 (Altschul and Gish, 1996, Local alignment statistics, Doolittle ed., Methods in Enzymology 266: 460-480; Altschul et al., 1990, Basic local alignment search tool, Journal of Molecular Biology 215: 403-410; Gish and States, 1993, Identification of protein coding regions by database similarity search, Nature Genetics 3: 266-272; Karlin and Altschul, 1993, Applications and statistics for multiple high-scoring segments in molecular sequences, Proc. Natl. Acad. Sci. USA 90: 5873-5877; all of which are incorporated by reference herein).

In all search programs in the suite the gapped alignment routines are integral to the database search itself. Gapping can be turned off if desired. The default penalty (O) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Once the amino acid sequences of turkey β1-AR and the target protein of unknown structure have been aligned, the structures of the conserved amino acids in the structural representation of the turkey β1-AR may be transferred to the corresponding amino acids of the target protein. For example, a tyrosine in the amino acid sequence of turkey β1-AR may be replaced by a phenylalanine, the corresponding homologous amino acid in the amino acid sequence of the target protein.

The structures of amino acids located in non-conserved regions may be assigned manually by using standard peptide geometries or by molecular simulation techniques, such as molecular dynamics. The final step in the process is accomplished by refining the entire structure using molecular dynamics and/or energy minimization. Typically, the predicted three dimensional structural representation will be one in which favourable interactions are formed within the target protein and/or so that a low energy conformation is formed (“High resolution structure prediction and the crystallographic phase problem” Qian et al (2007) Nature 450; 259-264; “State of the art in studying protein folding and protein structure production using molecular dynamics methods” Lee et al (2001) J of Mol Graph & Modelling 19(1): 146-149).

Whereas it is preferred to base homology modelling on homologous amino acid sequences, it is appreciated that some proteins have low sequence identity (e.g. family B and C GPCRs) and at the same time are very similar in structure. Therefore, where at least part of the structure of the target protein is known, homologous regions can also be identified by comparing structures directly.

Homology modelling as such is a technique well known in the art (see e.g. Greer, (Science, Vol. 228, (1985), 1055), and Blundell et al (Eur. J. Biochem, Vol. 172, (1988), 513)). The techniques described in these references, as well as other homology modelling techniques generally available in the art, may be used in performing the present invention.

Typically, homology modelling is performed using computer programs, for example SWISS-MODEL available through the Swiss Institute for Bioinformatics in Geneva, Switzerland; WHATIF available on EMBL servers; Schnare et al. (1996) J. Mol. Biol, 256: 701-719; Blundell et al. (1987) Nature 326: 347-352; Fetrow and Bryant (1993) Bio/Technology 11:479-484; Greer (1991) Methods in Enzymology 202: 239-252; and Johnson et al (1994) Crit. Rev. Biochem. Mol. Biol. 29:1-68. An example of homology modelling is described in Szklarz G. D (1997) Life Sci. 61: 2507-2520.

Thus, in an embodiment of the first aspect of the invention, the method further comprises aligning the amino acid sequence of the target protein of unknown structure with the amino acid sequence of turkey β1-AR listed in FIG. 7 to match homologous regions of the amino acid sequences, and subsequently modelling the structural representation of the target protein by modelling the structural representation of the matched homologous regions of the target protein on the corresponding regions of the β1-AR to obtain a three dimensional structural representation for the target protein that substantially preserves the structural representation of the matched homologous regions.

The invention therefore provides a method of predicting a three dimensional structural representation of a target protein of unknown structure, or part thereof, comprising:
providing the coordinates of the turkey β1-AR structure listed in Table A, Table B, Table C or Table D, optionally varied by a root mean square deviation of residue backbone atoms of not more than 1.235 Å, or selected coordinates thereof; aligning the amino acid sequence of a target protein of unknown structure or part thereof with the amino acid sequence of turkey β1-AR listed in FIG. 7 or part thereof to match homologous regions of the amino acid sequences;
modelling the structure of the matched homologous regions of the target protein on the corresponding regions of the turkey β1-AR structure as defined by Table A, Table B, Table C or Table D, optionally varied by a root mean square deviation of residue backbone atoms of not more than 1.235 Å, or selected coordinates thereof; and
predicting a three dimensional structural representation for the target protein which substantially preserves the structure of the matched homologous regions.

The coordinate data of Table A, Table B, Table C or Table D, or selected coordinates thereof, will be particularly advantageous for homology modelling of other GPCRs. For example, since the protein sequence of β1-AR and dopamine D2 receptor can be aligned relative to each other, it is possible to predict structural representations of the structures of the Dopamine D2 receptor, particularly in the regions of the transmembrane helices and ligand binding region, using the β1-AR coordinates.

The coordinate data of the turkey β1-AR can also be used to predict the crystal structure of target proteins where X-ray diffraction data or NMR spectroscopic data of the protein has been generated and requires interpretation in order to provide a structure.

A second aspect of the invention provides a method of predicting the three dimensional structural representation of a target protein of unknown structure, or part thereof, comprising: providing the coordinates of the turkey β1-AR structure listed in Table A, Table B, Table C or Table D, optionally varied by a root mean square deviation of residue backbone atoms of not more than 1.235 Å, or selected coordinates thereof; and either (a) positioning the coordinates in the crystal unit cell of the protein so as to predict its structural representation, or (b) assigning NMR spectra peaks of the protein by manipulating the coordinates.

Thus, where X-ray crystallographic or NMR spectroscopic data is provided for a target protein of unknown structure, the coordinate data of Table A, Table B, Table C or Table D may be used to interpret that data to predict a likely structure using techniques well known in the art including phasing, in the case of X-ray crystallography, and assisting peak assignments in the case of NMR spectra.

A three dimensional structural representation of any part of any target protein that is sufficiently similar to any portion of the turkey β1-AR can be predicted by this method. Typically, the target protein or part thereof has at least 20% amino acid sequence identity with any portion of turkey β1-AR, such as at least 30% amino acid sequence identity or at least 40% or 50% or 60% or 70% or 80% or 90% sequence identity. For example, the coordinates may be used to predict the three-dimensional representations of other crystal forms of turkey β1-AR, other β1-ARs, β1-AR mutants or co-complexes of a β1-AR. Other suitable target proteins are as defined with respect to the first aspect of the invention.

One method that may be employed for these purposes is molecular replacement which is well known in the art and described, for example, in Evans & McCoy (Acta Cryst, 2008, D64:1-10), McCoy (Acta Cryst, 2007, D63:32-42) and McCoy et al (J of App Cryst, 2007, 40:658-674). Molecular replacement enables the solution of the crystallographic phase problem by providing initial estimates of the phases of the new structure from a previously known structure, as opposed to the other major methods for solving the phase problem, i.e. experimental methods (which measure the phase from isomorphous or anomalous differences) or direct methods (which use mathematical relationships between reflection triplets and quartets to bootstrap a phase set for all reflections from phases for a small or random ‘seed’ set of reflections.) Compared to molecular replacement, such methods are time consuming and generally hinder the solution of crystal structures. Thus molecular replacement provides an accurate structural form for an unknown crystal more quickly and efficiently than attempting to determine such information ab initio.

Accordingly, the invention involves generating a preliminary model of a target protein whose structure coordinates are unknown, by orienting and positioning the relevant portion of the turkey β1-AR according to Table A, Table B, Table C or Table D within the unit cell of a crystal of the target protein so as best to account for the observed X-ray diffraction pattern of the crystal of the target protein. Phases can be calculated from this model and combined with the observed X-ray diffraction pattern amplitudes to generate an electron density map of the target protein's structure. This, in turn, can be subjected to any well-known model building and structure refinement techniques to provide a final, accurate structural representation of the target protein (E. Lattman, “Use of the Rotation and Translation Functions”, in Meth. Enzymol., 115, pp. 55-77 (1985); M. G. Rossmann, ed., “The Molecular Replacement Method”, Int. Sci. Rev. Ser., No. 13, Gordon & Breach, New York (1972)).

Thus the invention includes a method of predicting a three dimensional structural representation of a target protein of unknown structure, or part thereof, comprising: providing the coordinates of the turkey β1-AR structure, optionally varied by a root mean square deviation of residue backbone atoms of not more than 1.235 Å, or selected coordinates thereof; providing an X-ray diffraction pattern of the target protein; and using the coordinates to predict at least part of the structure coordinates of the target protein.

In an embodiment, the X-ray diffraction pattern of the target protein is provided by crystallising the target protein unknown structure; and generating an X-ray diffraction pattern from the crystallised target protein. Thus, the invention also provides a method of method of predicting a three dimensional structural representation of a target protein of unknown structure comprising the steps of (a) crystallising the target protein; (b) generating an X-ray diffraction pattern from the crystallised target protein; (c) applying the coordinates of the turkey β1-AR structure, optionally varied by a root mean square deviation of residue backbone atoms of not more than 1.235 Å, or selected coordinates thereof, to the X-ray diffraction pattern to generate a three-dimensional electron density map of the target protein, or part thereof; and (d) predicting a three dimensional structural representation of the target protein from the three-dimensional electron density map.

Examples of computer programs known in the art for performing molecular replacement include CNX (Brunger A T.; Adams P. D.; Rice L. M., Current Opinion in Structural Biology, Volume 8, Issue 5, October 1998, Pages 606-611 (also commercially available from Accelrys San Diego, Calif.), MOLREP (A. Vagin, A. Teplyakov, MOLREP: an automated program for molecular replacement, J Appl Cryst (1997) 30, 1022-1025, part of the CCP4 suite) or AMoRe (Navaza, J. (1994). AMoRe: an automated package for molecular replacement. Acta Cryst A50, 157-163).

Preferred selected coordinates of the turkey β1-AR are as defined above with respect to the first aspect of the invention.

The invention may also be used to assign peaks of NMR spectra of target proteins, by manipulation of the data of Table A, Table B, Table C or Table D (J Magn Reson (2002) 157(1): 119-23).

The coordinates of the β1-AR of Table A, Table B, Table C or Table D optionally varied by a root mean square deviation of residue backbone atoms of not more than 1.235 Å, or selected coordinates thereof may be used in the provision, design, modification or analysis of binding partners of β1-ARs. Such a use will be important in drug design.

By β1-AR we mean any β1-AR which has at least 75% sequence identity with turkey β1-AR, including turkey β1-AR as well as β1-AR from other species and mutants thereof. For example, human β1-AR has 82% amino acid sequence identity with turkey β1-AR. Therefore it is preferred if the β1-AR has at least 82% amino acid sequence identity to turkey β1-AR, more preferably at least 85%, 90%, 95% or 99% amino acid sequence identity.

By “binding partner” we mean any molecule that binds to a β1-AR. Preferably, the molecule binds selectively to the β1-AR. For example, it is preferred if the binding partner has a K_{d} value (dissociation constant) which is at least five or ten times lower (i.e. higher affinity) than for at least one other β-AR (e.g. β2-AR or β3-AR), and preferably more than 100 or 500 times lower. More preferably, the binding partner of a β1-AR has a K_{d} value more than 1000 or 5000 times lower than for at least one other β-AR. However, it will be appreciated that the limits will vary dependent upon the nature of the binding partner. Thus, typically, for small molecule binding partners, the binding partner typically has a K_{d} value which is at least 50 times or 100 times lower than for at least one other β-AR. Typically, for antibody binding partners, the binding partner typically has a K_{d} value which is at least 500 or 1000 times lower than for at least one other β-AR.

K_{d} values can be determined readily using methods well known in the art and as described, for example, below.

At equilibrium Kd=[R][L]/[RL]

where the terms in brackets represent the concentration of
Receptor-ligand complexes [RL],
unbound receptor [R], and
unbound (“free”) ligand [L].

In order to determine the K_{d} the value of these terms must be known. Since the concentration of receptor is not usually known then the Hill-Langmuir equation is used where

Fractional occupancy=[L]/[L]+K_{d}.

In order to experimentally determine a K_{d} then, the concentration of free ligand and bound ligand at equilibrium must be known. Typically, this can be done by using a radio-labelled or fluorescently labelled ligand which is incubated with the receptor (present in whole cells or homogenised membranes) until equilibrium is reached. The amount of free ligand vs bound ligand must then be determined by separating the signal from bound vs free ligand. In the case of a radioligand this can be done by centrifugation or filtration to separate bound ligand present on whole cells or membranes from free ligand in solution. Alternatively a scintillation proximity assay is used. In this assay the receptor (in membranes) is bound to a bead containing scintillant and a signal is only detected by the proximity of the radioligand bound to the receptor immobilised on the bead.

The binding partner may be any of a polypeptide; an anticalin; a peptide; an antibody; a chimeric antibody; a single chain antibody; an aptamer; a darpin; a Fab, F(ab′)₂, Fv, ScFv or dAb antibody fragment; a small molecule; a natural product; an affibody; a peptidomimetic; a nucleic acid; a peptide nucleic acid molecule; a lipid; a carbohydrate; a protein based on a modular framework including ankyrin repeat proteins, armadillo repeat proteins, leucine rich proteins, tetrariopeptide repeat proteins or Designed Ankyrin Repeat Proteins (DARPins); a protein based on lipocalin or fibronectin domains or Affilin scaffolds based on either human gamma crystalline or human ubiquitin; a G protein; an RGS protein; an arrestin; a GPCR kinase; a receptor tyrosine kinase; a RAMP; a NSF; a GPCR; an NMDA receptor subunit NR1 or NR2a; calcyon; or a fragment or derivative thereof that binds to β1-AR.

It will be appreciated that the coordinates of the invention will also be useful in the analysis of solvent and ion interactions with a β1-AR, which are important factors in drug design. Thus the binding partner may be a solvent molecule, for example water or acetonitrile, or an ion, for example a sodium ion or a protein.

It is particularly preferred if the binding partner is a small molecule with a molecule weight less than 5000 daltons, for example less than 4000, 3000, 2000 or 1000 daltons, or with a molecule weight less than 500 daltons, for example less than 450 daltons, 400 daltons, 350 daltons, 300 daltons, 250 daltons, 200 daltons, 150 daltons, 100 daltons, 50 daltons or 10 daltons.

It is further preferred if the binding partner causes a change (i.e a modulation) in the level of biological activity of the β1-AR, i.e. it has functional agonist or antagonist activity, and therefore may have the potential to be a candidate drug. Thus, the binding partner may be any of a full agonist, a partial agonist, an inverse agonist or an antagonist of β1-AR.

Accordingly, a third aspect of the invention provides a method for selecting or designing one or more binding partners of β1-AR comprising using molecular modelling means to select or design one or more binding partners of β1-AR, wherein the three-dimensional structural representation of at least part of turkey β1-AR, as defined by the coordinates of turkey β1-AR of Table A, Table B, Table C or Table D, optionally varied by a root mean square deviation of residue backbone atoms of not more than 1.235 Å or selected coordinates thereof, is compared with a three-dimensional structural representation of one or more candidate binding partners, and one or more binding partners that are predicted to interact with β1-AR are selected.

In order to provide a three-dimensional structural representation of a candidate binding partner, the binding partner structural representation may be modelled in three dimensions using commercially available software for this purpose or, if its crystal structure is available, the coordinates of the structure may be used to provide a structural representation of the binding partner.

The design of binding partners that bind to a β1-AR generally involves consideration of two factors.

First, the binding partner must be capable of physically and structurally associating with parts or all of a β1-AR binding region. Non-covalent molecular interactions important in this association include hydrogen bonding, van der Waals interactions, hydrophobic interactions and electrostatic interactions.

Second, the binding partner must be able to assume a conformation that allows it to associate with a β1-AR binding region directly. Although certain portions of the binding partner will not directly participate in these associations, those portions of the binding partner may still influence the overall conformation of the molecule. This, in turn, may have a significant impact on potency. Such conformational requirements include the overall three-dimensional structure and orientation of the binding partner in relation to all or a portion of the binding region, or the spacing between functional groups of a binding partner comprising several binding partners that directly interact with the β1-AR.

Thus it will be appreciated that selected coordinates which represent a binding region of the turkey β1-AR, e.g. atoms from amino acid residues contributing to the ligand binding site including amino acid residues 117, 118, 121, 122, 125, 201, 203, 207, 211, 215, 306, 307, 310 and 329 and amino acid residues 172 and 325 may be used. Selected coordinates representing an extracellular face would be useful to select or design for antibodies, and selected coordinates representing an intracellular face would be useful to select or design for natural binding partners such as G proteins.

Additional preferences for the selected coordinates are as defined above with respect to the first aspect of the invention.

Designing of binding partners can generally be achieved in two ways, either by the step wise assembly of a binding partner or by the de novo synthesis of a binding partner.

With respect to the step-wise assembly of a binding partner, several methods may be used. Typically the process begins by visual inspection of, for example, any of the binding regions on a computer representation of the turkey β1-AR as defined by the coordinates in Table A, Table B, Table C or Table D optionally varied within a rmsd of residue backbone atoms of not more than 1.235 Å, or selected coordinates thereof. Selected binding partners, or fragments or moieties thereof may then be positioned in a variety of orientations, or docked, within the binding region. Docking may be accomplished using software such as QUANTA and Sybyl (Tripos Associates, St. Louis, Mo.), followed by, or performed simultaneously with, energy minimization, rigid-body minimization (Gshwend, supra) and molecular dynamics with standard molecular mechanics force fields, such as CHARMM and AMBER.

Specialized computer programs may also assist in the process of selecting binding partners or fragments or moieties thereof. These include: 1. GRID (P. J. Goodford, “A Computational Procedure for Determining Energetically Favorable Binding Sites on Biologically Important Macromolecules”, J. Med. Chem., 28, pp. 849-857 (1985)). GRID is available from Oxford University, Oxford, UK. 2. MCSS (A. Miranker et al., “Functionality Maps of Binding Sites: A Multiple Copy Simultaneous Search Method.”Proteins: Structure, Function and Genetics, 11, pp. 29-34 (1991)). MCSS is available from Molecular Simulations, San Diego, Calif. 3. AUTODOCK (D. S. Goodsell et al., “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif. 4. DOCK (I. D. Kuntz et al., “A Geometric Approach to Macromolecule-Ligand Interactions”, J. Mol. Biol., 161, pp. 269-288 (1982)). DOCK is available from University of California, San Francisco, Calif.

Once suitable binding partners or fragments have been selected, they may be assembled into a single compound or complex. Assembly may be preceded by visual inspection of the relationship of the fragments to each other on the three-dimensional image displayed on a computer screen in relation to the structure coordinates of the turkey β1-AR. This would be followed by manual model building using software such as QUANTA or Sybyl.

Useful programs to aid one of skill in the art in connecting the individual chemical entities or fragments include: 1. CAVEAT (P. A. Bartlett et al., “CAVEAT: A Program to Facilitate the Structure-Derived Design of Biologically Active Molecules”, in “Molecular Recognition in Chemical and Biological Problems”, Special Pub., Royal Chem. Soc., 78, pp. 182-196 (1989); G. Lauri and P. A. Bartlett, “CAVEAT: a Program to Facilitate the Design of Organic Molecules”, J. Comput. Aided Mol. Des., 8, pp. 51-66 (1994)). CAVEAT is available from the University of California, Berkeley, Calif.; 2. 3D Database systems such as ISIS (MDL Information Systems, San Leandro, Calif.). This area is reviewed in Y. C. Martin, “3D Database Searching in Drug Design”, J. Med. Chem., 35, pp. 2145-2154 (1992); and 3. HOOK (M. B. Eisen et al., “HOOK: A Program for Finding Novel Molecular Architectures that Satisfy the Chemical and Steric Requirements of a Macromolecule Binding Site”, Proteins: Struct., Funct., Genet., 19, pp. 199-221 (1994). HOOK is available from Molecular Simulations, San Diego, Calif.

Thus the invention includes a method of designing a binding partner of a β1-AR comprising the steps of: (a) providing a structural representation of a β1-AR binding region as defined by the coordinates of turkey β1-AR of Table A, Table B, Table C or Table D, optionally varied by a root mean square deviation of residue backbone atoms of not more than 1.235 Å or selected coordinates thereof (b) using computational means to dock a three dimensional structural representation of a first binding partner in part of the binding region; (c) docking at least a second binding partner in another part of the binding region; (d) quantifying the interaction energy between the first or second binding partner and part of the binding region; (e) repeating steps (b) to (d) with another first and second binding partner, selecting a first and a second binding partner based on the quantified interaction energy of all of said first and second binding partners; (f) optionally, visually inspecting the relationship of the first and second binding partner to each other in relation to the binding region; and (g) assembling the first and second binding partners into a one binding partner that interacts with the binding region by model building.

As an alternative to the step-wise assembly of binding partners, binding partners may be designed as a whole or “de novo” using either an empty binding region or optionally including some portion(s) of a known binding partner(s). There are many de novo ligand design methods including: 1. LUDI (H.-J. Bohm, “The Computer Program LUDI: A New Method for the De Novo Design of Enzyme Inhibitors”, J. Comp. Aid. Molec. Design, 6, pp. 61-78 (1992)). LUDI is available from Molecular Simulations Incorporated, San Diego, Calif.; 2. LEGEND (Y. Nishibata et al., Tetrahedron, 47, p. 8985 (1991)). LEGEND is available from Molecular Simulations Incorporated, San Diego, Calif.; 3. LeapFrog (available from Tripos Associates, St. Louis, Mo.); and 4. SPROUT (V. Gillet et al., “SPROUT: A Program for Structure Generation)”, J. Comput. Aided Mol. Design, 7, pp. 127-153 (1993)). SPROUT is available from the University of Leeds, UK.

Other molecular modelling techniques may also be employed in accordance with this invention (see, e.g., N. C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)).

In addition to the methods described above in relation to the design of binding partners, other computer-based methods are available to select for binding partners that interact with β1-AR.

For example the invention involves the computational screening of small molecule databases for binding partners that can bind in whole, or in part, to the turkey β1-AR.

In this screening, the quality of fit of such binding partners to a binding region of a β1-AR site as defined by the coordinates of turkey β1-AR of Table A, Table B, Table. C or Table D, optionally varied by a root mean square deviation of residue backbone atoms of not more than 1.235 Å or selected coordinates thereof, may be judged either by shape complementarity or by estimated interaction energy (E. C. Meng et. al., J. Comp. Chem., 13, pp. 505-524 (1992)).

For example, selection may involve using a computer for selecting an orientation of a binding partner with a favourable shape complementarity in a binding region comprising the steps of: (a) providing the coordinates of turkey β1-AR of Table A, Table B, Table C or Table D, optionally varied by a root mean square deviation of residue backbone atoms of not more than 1.235 Å or selected coordinates thereof and a three-dimensional structural representation of one or more candidate binding partners; (b) employing computational means to dock a first binding partner in the binding region; (c) quantitating the contact score of the binding partner in different orientions; and (d) selecting an orientation with the highest contact score.

The docking may be facilitated by the contact score. The method may further comprise the step of generating a three-dimensional structural repsentation of the binding region and binding partner bound therein prior to step (b).

The method may further comprise the steps of: (e) repeating steps (b) through (d) with a second binding partner; and (f) selecting at least one of the first or second binding partner that has a higher contact score based on the quantitated contact score of the first or second binding partner.

In another embodiment, selection may involve using a computer for selecting an orientation of a binding partner that interacts favourably with a binding region comprising; a) providing the coordinates of turkey β1-AR of Table A, Table B, Table C or Table D, optionally varied by a root mean square deviation of residue backbone atoms of not more than 1.235 Å or selected coordinates thereof; b) employing computational means to dock a first binding partner in the binding region; c) quantitating the interaction energy between the binding partner and all or part of a binding region for different orientations of the binding partner; and d) selecting the orientation of the binding partner with the most favorable interaction energy.

The docking may be facilitated by the quantitated interaction energy and energy minimization with or without molecular dynamics simulations may be performed simultaneously with or following step (b).

The method may further comprise the steps of: (e) repeating steps (b) through (d) with a second binding partner; and (f) selecting at least one of the first or second binding partner that interacts more favourably with a binding region based on the quantitated interaction energy of the first or second binding partner.

In another embodiment, selection may involve screening a binding partner to associate at a deformation energy of binding of less than −7 kcal/mol with a β1-AR binding region comprising: (a) providing the coordinates of turkey rβ1-AR of Table A, Table B, Table C or Table D, optionally varied by a root mean square deviation of residue backbone atoms of not more than 1.235 Å or selected coordinates thereof and employing computational means which utilise coordinates to dock the binding partner into a binding region; (b) quantifying the deformation energy of binding between the binding partner and the binding region; and (d) selecting a binding partner that associates with a β1-AR binding region at a deformation energy of binding of less than −7 kcal/mol.

It is appreciated that in some instances high throughput screening of binding partners is preferred and that methods of the invention may be used as “library screening” methods, a term well known to those skilled in the art. Thus, the binding partner may be a library of binding partners. For example, the library may be a peptide or protein library produced, for example, by ribosome display or an antibody library prepared either in vivo, ex vivo or in vitro. Methodologies for preparing and screening such libraries are known in the art.

Determination of the three-dimensional structure of the turkey β1-AR provides important information about the binding sites of β1-ARs, particularly when comparisons are made with other β-ARs. This information may then be used for rational design and modification of β1-AR binding partners, e.g. by computational techniques which identify possible binding ligands for the binding sites, by enabling linked-fragment approaches to drug design, and by enabling the identification and location of bound ligands using X-ray crystallographic analysis. These techniques are discussed in more detail below.

Thus as a result of the determination of the turkey β1-AR three-dimensional structure, more purely computational techniques for rational drug design may also be used to design structures whose interaction with β1-AR is better understood (for an overview of these techniques see e.g. Walters et al (Drug Discovery Today, Vol. 3, No. 4, (1998), 160-178; Abagyan, R.; Totrov, M. Curr. Opin. Chem. Biol. 2001, 5, 375-382). For example, automated ligand-receptor docking programs (discussed e.g. by Jones et al. in Current Opinion in Biotechnology, Vol. 6, (1995), 652-656 and Halperin, I.; Ma, B.; Wolfson, H.; Nussinov, R. Proteins 2002, 47, 409-443), which require accurate information on the atomic coordinates of target receptors may be used.

The aspects of the invention described herein which utilize the β1-AR structure in silico may be equally applied to both the turkey β1-AR structure of Table A, Table B, Table C or Table D, optionally varied by a root mean square deviation of residue backbone atoms of not more than 1.235 Å, or selected coordinates thereof; and predicting the three-dimensional structural representation of the target protein, or part thereof, by modelling the structural representation on all or the selected coordinates of the turkey β1-AR or selected coordinates thereof and the models of target proteins obtained by the first and second aspects of the invention. Thus having determined a conformation of a target protein, for example a β1-AR, by the methods described above, such a conformation may be used in a computer-based method of rational drug design as described herein. In addition, the availability of the structure of the turkey β1-AR will allow the generation of highly predictive pharmacophore models for virtual library screening or ligand design.

Accordingly, a fourth aspect of the invention provides a method for the analysis of the interaction of one or more binding partners with β1-AR, comprising: providing a three dimensional structural representation of β1-AR as defined by the coordinates of the turkey β1-AR structure of Table A, Table B, Table C or Table D, optionally varied by a root mean square deviation of residue backbone atoms of not more than 1.235 Å, or selected coordinates thereof; providing a three dimensional structural representation of one or more binding partners to be fitted to the structural representation of β1-AR or selected coordinates thereof; and fitting the one of more binding partners to said structure.

This method of the invention is generally applicable for the analysis of known binding partners of β1-AR, the development or discovery of binding partners of β1-AR, the modification of binding partners of β1-AR e.g. to improve or modify one or more of their properties, and the like. Moreover, the methods of the invention are useful in identifying binding partners than are selective for β1-ARs over β2-ARs. For example, comparing corresponding binding regions between β1-AR and β2-AR will facilitate the design of β1-AR specific binding partners.

It will be desirable to model a sufficient number of atoms of the β1-AR as defined by the coordinates of Table A, Table B, Table C or Table D optionally varied by a root mean square deviation of residue backbone atoms of not more than 1.235 Å, or selected coordinates thereof, which represent a binding region, e.g. atoms from amino acid residues contributing to the ligand binding site including amino acid residues 117, 118, 121, 122, 125, 201, 203, 207, 211, 215, 306, 307, 310 and 329. Although every different binding partner bound by β1-AR may interact with different parts of the binding region of the protein, the structure of the turkey β1-AR allows the identification of a number of particular sites which are likely to be involved in many of the interactions of β1-AR with a drug candidate. Additional preferred selected coordinates are as described as above with respect to the first aspect of the invention.

In order to provide a three-dimensional structural representation of a binding partner to be fitted to the turkey β1-AR structure, the binding partner structural representation may be modelled in three dimensions using commercially available software for this purpose or, if its crystal structure is available, the coordinates of the structure may be used to provide a structural representation of the binding partner for fitting to the turkey β1-AR structure of the invention.

By “fitting”, is meant determining by automatic, or semi-automatic means, interactions between one or more atoms of a candidate binding partner and at least one atom of the turkey β1-AR structure of the invention, and calculating the extent to which such interactions are stable. Interactions include attraction and repulsion, brought about by charge, steric, lipophilic, considerations and the like. Charge and steric interactions of this type can be modelled computationally. An example of such computation would be via a force field such as Amber (Cornell et al., A Second Generation Force Field for the Simulation of Proteins, Nucleic Acids, and Organic Molecules, Journal of the American Chemical Society, (1995), 117(19), 5179-97) which would assign partial charges to atoms on the protein and binding partner and evaluate the electrostatic interaction energy between a protein and binding partner atom using the Coulomb potential. The Amber force field would also assign van der Waals energy terms to assess the attractive and repulsive steric interactions between two atoms. Lipophilic interactions can be modeled using a variety of means. For example the ChemScore function (Eldridge M D; Murray C W; Auton T R; Paolini G V; Mee R P Empirical scoring functions: I. The development of a fast empirical scoring function to estimate the binding affinity of binding partners in receptor complexes, Journal of computer-aided molecular design (1997 September), 11 (5), 425-45) assigns protein and binding partner atoms as hydrophobic or polar, and a favourable energy term is specified for the interaction between two hydrophobic atoms. Other methods of assessing the hydrophobic contributions to ligand binding are available and these would be known to one skilled in the art. Other methods of assessing interactions are available and would be known to one skilled in the art of designing molecules. Various computer-based methods for fitting are described further herein.

More specifically, the interaction of a binding partner with the turkey β1-AR structure of the invention can be examined through the use of computer modelling using a docking program such as GOLD (Jones et al., J. Mol. Biol., 245, 43-53 (1995), Jones et al., J. Mol. Biol., 267, 727-748 (1997)), GRAMM (Vakser, I. A., Proteins, Suppl., 1: 226-230 (1997)), DOCK (Kuntz et al, (1982) J. Mol. Biol., 161, 269-288; Makino et al, (1997) J. Comput. Chem., 18, 1812-1825), AUTODOCK (Goodsell et al, (1990) Proteins, 8, 195-202, Morris et al, (1998) J. Comput. Chem., 19, 1639-1662.), FlexX, (Rarey et al, (1996) J. Mol. Biol., 261, 470-489) or ICM (Abagyan et al, (1994) J. Comput. Chem., 15, 488-506). This procedure can include computer fitting of binding partners to the turkey β1-AR structure to ascertain how well the shape and the chemical structure of the binding partner will bind to a β1-AR.

Thus the invention includes a method for the analysis of the interaction of one or more binding partners with β1-AR comprising (a) constructing a computer representation of a binding region of the turkey β1-AR as defined by the coordinates of turkey β1-AR of Table A, Table B, Table C or Table D, optionally varied by a root mean square deviation of residue backbone atoms of not more than 1.235 Å or selected coordinates thereof (b) selecting a binding partner to be evaluated by a method selected from the group consisting of assembling said binding partner; selecting a binding partner from a small molecule database; de novo ligand design of the binding partner; and modifying a known agonist or inhibitor, or a portion thereof, of a β1-AR or homologue thereof; (c) employing computational means to dock said binding partner to be evaluated in a binding region in order to provide an energy-minimized configuration of the binding partner in a binding region; and (d) evaluating the results of said docking to quantify the interaction energy between said, binding partner and the binding region.

Also computer-assisted, manual examination of the binding region structure of the turkey β1-AR may be performed. The use of programs such as GRID (Goodford, (1985) J. Med. Chem., 28, 849-857)—a program that determines probable interaction sites between molecules with various functional groups and an enzyme surface—may also be used to analyse a binding region to predict, for example, the types of modifications which will alter the rate of metabolism of a binding partner.

Computer programs can be employed to estimate the attraction, repulsion, and steric hindrance of the turkey β1-AR structure and a binding partner.

If more than one turkey β1-AR binding region is characterized and a plurality of respective smaller molecular fragments are designed or selected, a binding partner may be formed by linking the respective small molecular fragments into a single binding partner, which maintains the relative positions and orientations of the respective small molecular fragments at the binding sites. The single larger binding partner may be formed as a real molecule or by computer modelling. Detailed structural information can then be obtained about the binding of the binding partner to β1-AR, and in the light of this information adjustments can be made to the structure or functionality of the binding partner, e.g. to alter its interaction with β1-AR. The above steps may be repeated and re-repeated as necessary.

Thus, the three dimensional structural representation of the one or more binding partners of the third and fourth aspects of the invention may be obtained by: providing structural representations of a plurality of molecular fragments; fitting the structural representation of each of the molecular fragments to the coordinates of the turkey β1-AR structural representation of Table A, Table B, Table C or Table D, optionally varied by a root mean square deviation of residue C-α atoms of not more than 1.235 Å, or selected coordinates thereof; and assembling the representations of the molecular fragments into one or more representations of single molecules to provide the three-dimensional structural representation of one or more candidate binding partners.

Typically the binding partner or molecule fragment is fitted to at least 5 or 10 non-hydrogen atoms of the turkey β1-AR structure, preferably at least 20, 30, 40, 50, 60, 70, 80 or 90 non-hydrogen atoms and more preferably at least 100, 150, 200, 250, 300, 350, 400, 450, or 500 atoms and even more preferably at least 600, 700, 800, 900, 1000, 1100, 1200, 1300, 1400, 1500, 1600, 1700, 1800, 1900, 2000, 2100 or 2200 non-hydrogen atoms.

The invention includes screening methods to identify drugs or lead compounds of use in treating a disease or condition. For example, large numbers of binding partners, for example in a chemical database, can be screened for their ability to bind β1-AR.

It is appreciated that in the methods described herein, which may be drug screening methods, a term well known to those skilled in the art, the binding partner may be a drug-like compound or lead compound for the development of a drug-like compound.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons (such as less than 560 daltons) and which may be water-soluble. A drug-like compound may additionally exhibit features of selective interaction with a particular protein or proteins and be bioavailable and/or able to penetrate target cellular membranes or the blood:brain barrier, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics.

Thus in one embodiment of the methods of third and fourth aspects of the invention, the methods further comprise modifying the structural representation of the binding partner so as to increase or decrease their interaction with β1-AR.

For example, once a binding partner has been designed or selected by the above methods, the efficiency with which that binding partner may bind to a β1-AR may be tested and optimized, for example by computational evaluation. For example, a binding partner designed or selected as binding to a β1-AR may be further computationally optimized so that in its bound state it would preferably lack repulsive electrostatic interaction with the target β1-AR and with the surrounding water molecules. Such non-complementary electrostatic interactions include repulsive charge-charge, dipole-dipole and charge-dipole interactions.

Furthermore, it is often desired that binding partners demonstrate a relatively small difference in energy between the bound and free states (i.e., a small deformation energy of binding). Thus, binding partners may be designed with a deformation energy of binding of not greater than about 10 kcal/mole, more preferably, not greater than 7 kcal/mole. Binding partners may interact with the binding region in more than one conformation that is similar in overall binding energy. In those cases, the deformation energy of binding is taken to be the difference between the energy of the free binding partner and the average energy of the conformations observed when the binding partner binds to the protein.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. .COPYRGT. 1995); AMBER, version 4.1 (P. A. Kollman, University of California at San Francisco, .COPYRGT. 1995); QUANTA/CHARMM (Molecular Simulations, Inc., San Diego, Calif. COPYRGT. 1998); Insight II/Discover (Molecular Simulations, Inc., San Diego, Calif. COPYRGT. 1998); DelPhi (Molecular Simulations, Inc., San Diego, Calif. COPYRGT. 1998); and AMSOL (Quantum Chemistry Program Exchange, Indiana University). These programs may be implemented, for instance, using a Silicon Graphics workstation such as an Indigo2 with “IMPACT” graphics. Other hardware systems and software packages will be known to those skilled in the art.

By modifying the structural representation we include, for example, adding molecular scaffolding, adding or varying functional groups, or connecting the molecule with other molecules (e.g. using a fragment linking approach) such that the chemical structure of the binding partner is changed while its original binding to β1-AR capability is increased or decreased. Such optimisation is regularly undertaken during drug development programmes to e.g. enhance potency, promote pharmacological acceptability, increase chemical stability etc. of lead compounds.

Examples of modifications include substitutions or removal of groups containing residues which interact with the amino acid side chain groups of the β1-AR structure of the invention. For example, the replacements may include the addition or removal of groups in order to decrease or increase the charge of a group in a binding partner, the replacement of a charge group with a group of the opposite charge, or the replacement of a hydrophobic group with a hydrophilic group or vice versa. It will be understood that these are only examples of the type of substitutions considered by medicinal chemists in the development of new pharmaceutical compounds and other modifications may be made, depending upon the nature of the starting binding partner and its activity.

The potential binding effect of a binding partner on β1-AR may be analysed prior to its actual synthesis and testing by the use of computer modeling techniques. If the theoretical structure of the given entity suggests insufficient interaction and association between it and the β1-AR, testing of the entity is obviated. However, if computer modelling indicates a strong interaction, the molecule may then be synthesized and tested for its ability to bind to a β1-AR. In this manner, synthesis of inoperative compounds may be avoided.

Thus in a further embodiment of the third and fourth aspects of the invention, the methods further comprise the steps of obtaining or synthesising the one or more binding partners of a β1-AR; and optionally contacting the one or more binding partners with a β1-AR to determine the ability of the one or more binding partners to interact with the β1-AR.

Various methods may be used to determine binding between a β1-AR and a binding partner including, for example, enzyme linked immunosorbent assays (ELISA), surface plasmon resonance assays, chip-based assays, immunocytofluorescence, yeast two-hybrid technology and phage display which are common practice in the art and are described, for example, in Plant et al (1995) Analyt Biochem, 226(2), 342-348 and Sambrook et al (2001) Molecular Cloning A Laboratory Manual. Third Edition. Cold Spring Harbor Laboratory Press, Cold Spring Harbor, N.Y. Other methods of detecting binding, between a β1-AR and a binding partner include ultrafiltration with ion spray mass spectroscopy/HPLC methods or other physical and analytical methods. Fluorescence Energy Resonance Transfer (FRET) methods, for example, well known to those skilled in the art, may be used, in which binding of two fluorescent labelled entities may be measured by measuring the interaction of the fluorescent labels when in close proximity to each other.

Once computer modelling has indicated that a binding partner has a strong interaction, it is appreciated that it may be desirable to crystallise a complex of the β1-AR with that binding partner and analyse its interaction further by X-ray crystallography.

Thus in a further embodiment of the third and fourth aspects of the invention, the methods further comprise the steps of obtaining or synthesising the one or more binding partners of a β1-AR; forming one or more complexes of the β1-AR and the one or more binding partners; and analysing the one or more complexes by X-ray crystallography to determine the ability of the one or more binding partners to interact with β1-AR.

Thus, it will be appreciated that another particularly useful drug design technique enabled by this invention is iterative drug design. Iterative drug design is a method for optimizing associations between a protein and a binding partner by determining and evaluating the three-dimensional structures of successive sets of protein/compound complexes.

In iterative drug design, crystals of a series of proteins or protein complexes are obtained and then the three-dimensional structures of each crystal is solved. Such an approach provides insight into the association between the proteins and binding partners of each complex. This is accomplished by selecting candidate binding partners, obtaining crystals of this new protein/binding partner complex, solving the three-dimensional structure of the complex, and comparing the associations between the new protein/binding partner complex and previously solved protein/binding partner complexes. By observing how changes in the binding partner affected the protein/binding partner associations, these associations may be optimized.

In some cases, iterative drug design is carried out by forming successive protein-binding partner complexes and then crystallizing each new complex. High throughput crystallization assays may be used to find a new crystallization condition or to optimize the original protein or complex crystallization condition for the new complex. Alternatively, a pre-formed protein crystal may be soaked in the presence of a binding partner, thereby forming a protein/binding partner complex and obviating the need to crystallize each individual protein/binding partner complex.

The ability of a binding partner to modify β1-AR function may also be tested. For example the ability of a binding partner to modulate a β1-AR function could be tested by a number of well known standard methods, described extensively in the prior art.

In addition to in silico analysis and design, the interaction of one or more binding partners with a β1-AR may be analysed directly by X-ray crystallography experiments, wherein the coordinates of the turkey β1-AR of Table A, Table B, Table C or Table D optionally varied by a root mean square deviation of residue backbone atoms of not more than 1.235 Å, or selected coordinates thereof, are used to analyse the a crystal complex of the β1-AR and binding partner. This can provide high resolution information of the interaction and can also provide insights into a mechanism by which a binding partner exerts an agonistic or antagonistic function.

Accordingly, a fifth aspect of the invention provides a method for the analysis of the interaction of one or more binding partners with β1-AR, comprising: obtaining or synthesising one or more binding partners; forming one or more crystallised complexes of a β1-AR and a binding partner; and analysing the one or more complexes by X-ray crystallography by employing the coordinates of the turkey β1-AR structure, of Table A, Table B, Table C or Table D optionally varied by a root mean square deviation of residue backbone atoms of not more than 1.235 Å, or selected coordinates thereof, to determine the ability of the one or more binding partners to interact with the β1-AR.

Preferences for the selected coordinates in this and all subsequent aspects of the invention are as defined above with respect to the first aspect of the invention.

The analysis of such structures may employ X-ray crystallographic diffraction data from the complex and the coordinates of the turkey β1-AR structure, of Table A, Table B, Table C or Table D optionally varied by a root mean square deviation of residue backbone atoms of not more than 1.235 Å, or selected coordinates thereof, to generate a difference Fourier electron density map of the complex. The difference Fourier electron density map may then be analysed.

In one embodiment, the one or more crystallised complexes are formed by soaking a crystal of β1-AR with the binding partner to form a complex. Alternatively, the complexes may be obtained by cocrystallising the β1-AR with the binding partner. For example a purified β1-AR protein sample is incubated over a period of time (usually >1 hr) with a potential binding partner and the complex can then be screened for crystallization conditions. Alternatively, protein crystals containing a first binding partner can be back-soaked to remove this binding partner by placing the crystals into a stabilising solution in which the binding partner is not present. The resultant crystals can then be transferred into a second solution containing a second binding partner and used to produce an X-ray diffraction pattern of β1-AR complexed with the second binding partner.

The complexes can be analysed using X-ray diffraction methods, e.g. according to the approach described by Greer et al., (J of Medicinal Chemistry, Vol. 37, (1994), 1035-1054), and difference Fourier electron density maps can be calculated based on X-ray diffraction patterns of soaked or co-crystallized β1-AR and the solved structure of uncomplexed β1-AR. These maps can then be analysed e.g. to determine whether and where a particular ligand binds to β1-AR and/or changes the conformation of β1-AR.

Electron density maps can be calculated using programs such as those from the CCP4 computing package (Collaborative Computational Project 4. The CCP4 Suite: Programs for Protein Crystallography, Acta Crystallographica, D50, (1994), 760-763.). For map visualization and model building programs such as “0” (Jones et al., Acta Crystallographica, A47, (1991), 110-119) can be used.

All of the complexes referred to above may be studied using well-known X-ray diffraction techniques and may be refined against 1.5 to 3.5 A resolution X-ray data to an R value of about 0.30 or less using computer software, such as CNX (Brunger et al., Current Opinion in Structural Biology, Vol. 8, Issue 5, October 1998, 606-611, and commercially available from Accelrys, San Diego, Calif.)1 and as described by Blundell et al, (1976) and Methods in Enzymology, vol. 114 & 115, H. W. Wyckoff et al., eds., Academic Press (1985).

This information may thus be used to optimise known classes of β1-AR binding partners and to design and synthesize novel classes of β1-AR binding partners, particularly those which have agonistic or antagonistic properties, and to design drugs with modified β1-AR interactions.

In one approach, the structure of a binding partner bound to a β1-AR may be determined by experiment. This will provide a starting point in the analysis of the binding partner bound to β1-AR thus providing those of skill in the art with a detailed insight as to how that particular binding partner interacts with β1-AR and the mechanism by which it exerts any function effect.

Many of the techniques and approaches applied to structure-based drug design described above rely at some stage on X-ray analysis to identify the binding position of a binding partner in a ligand-protein complex. A common way of doing this is to perform X-ray crystallography on the complex, produce a difference Fourier electron density map, and associate a particular pattern of electron density with the binding partner. However, in order to produce the map (as explained e.g. by Blundell et al., in Protein Crystallography, Academic Press, New York, London and San Francisco, (1976)), it is necessary to know beforehand the protein three dimensional structure (or at least a set of structure factors for the protein crystal). Therefore, determination of the turkey β1-AR structure also allows difference Fourier electron density maps of β1-AR-binding partner complexes to be produced, determination of the binding position of the binding partner and hence may greatly assist the process of rational drug design.

Accordingly, a sixth aspect of the invention provides a method for predicting the three dimensional structure of a binding partner of unknown structure, or part thereof, which binds to β1-AR, comprising: providing the coordinates of the turkey β1-AR structure of Table A, Table B, Table C or Table D, optionally varied by a root mean square deviation of residue backbone atoms of not more than 1.235 Å, or selected coordinates thereof; providing an X-ray diffraction pattern of β1-AR complexed with the binding partner; and using the coordinates to predict at least part of the structure coordinates of the binding partner.

In one embodiment, the X-ray diffraction pattern is obtained from a crystal formed by soaking a crystal of β1-AR with the binding partner to form a complex. Alternatively, the X-ray diffraction pattern is obtained from a crystal formed by cocrystallising the β1-AR with the binding partner as described above. Alternatively, protein crystals containing a first binding partner can be back-soaked to remove this binding partner and the resultant crystals transferred into a second solution containing a second binding partner as described above.

A mixture of compounds may be soaked or co-crystallized with a turkey β1-AR crystal, wherein only one or some of the compounds may be expected to bind to the turkey β1-AR. The mixture of compounds may comprise a ligand known to bind to turkey β1-AR. As well as the structure of the complex, the identity of the complexing compound(s) is/are then determined.

Preferably, the methods of the previous aspects of the invention are computer-based. For example, typically the methods of the previous aspects of the invention make use of the computer systems and computer-readable storage mediums of the ninth and tenth aspects of the invention.

A seventh aspect of the invention provides a method for producing a binding partner of β1-AR comprising: identifying a binding partner according to the third, fourth, fifth or sixth aspects of the invention and synthesising the binding partner.

The binding partner may be synthesised using any suitable technique known in the art including, for example, the techniques of synthetic chemistry, organic chemistry and molecular biology.

It will be appreciated that it may be desirable to test the binding partner in an in vivo or in vitro biological system in order to determine its binding and/or activity and/or its effectiveness. For example, its binding to a β1-AR may be assessed using any suitable binding assay known in the art including the examples described above.

Moreover, its effect on β1-AR function in an in vivo or in vitro assay may be tested. For example, the effect of the binding partner on the β1-AR signalling pathway may be determined. For example, the activity may be measured by using a reporter gene to measure the activity of the β1-AR signalling pathway. By a reporter gene we include genes which encode a reporter protein whose activity may easily be assayed, for example β-galactosidase, chloramphenicol acetyl transferase (CAT) gene, luciferase or Green Fluorescent Protein (see, for example, Tan et al, 1996 EMBO J. 15(17): 4629-42). Several techniques are available in the art to detect and measure, expression of a reporter gene which would be suitable for use in, the present invention. Many of these are available in kits both for determining expression in vitro and in vivo. Alternatively, signalling may be assayed by the analysis of downstream targets. For example, a particular protein whose expression is known to be under the control of a specific signalling pathway may be quantified. Protein levels in biological samples can be determined using any suitable method known in the art. For example, protein concentration can be studied by a range of antibody based methods including immunoassays, such as ELISAs, western blotting and radioimmunoassays

An eight aspect of the invention provides a binding partner produced by the method of the seventh aspect of the invention.

Following identification of a binding partner, it may be manufactured and/or used in the preparation, i.e. manufacture or formulation, of a composition such as a medicament, pharmaceutical composition or drug. These may be administered to individuals.

Accordingly, the invention includes a method for producing a medicament, pharmaceutical composition or drug, the process comprising: (a) providing a binding partner according to the eighth aspect of the invention and (b) preparing a medicament, pharmaceutical composition or drug containing the binding partner.

The medicaments may be used to treat hypertension and cardiovascular disease (including congestive heart failure) and cardiovascular disease in the context of metabolic disease (eg diabetes and/or obesity) and/or respiratory disease (eg COPD (chronic obstructive pulmonary disease)).

The invention also provides systems, particularly a computer system, intended to generate structures and/or perform optimisation of binding partner which interact with β1-AR, β1-AR homologues or analogues, complexes of β1-AR with binding partners, or complexes of β1-AR homologues or analogues with binding partners.

Accordingly, a ninth aspect of the invention provides a computer system, intended to generate three dimensional structural representations of β1-AR, β1-AR homologues or analogues, complexes of β1-AR with binding partners, or complexes of β1-AR homologues or analogues with binding partners, or, to analyse or optimise binding of binding partners to said β1-AR or homologues or analogues, or complexes thereof, the system containing computer-readable data comprising one or more of:
(a) the coordinates of the turkey β1-AR structure of Table A, Table B, Table C or Table D, optionally varied by a root mean square deviation of residue backbone atoms of not more than 1.235 Å, or selected coordinates thereof;
(b) the coordinates of a target β1-AR homologue or analogue generated by homology modelling of the target based on the data in (a);
(c) the coordinates of a binding partner generated by interpreting X-ray crystallographic data or NMR data by reference to the coordinates of the turkey β1-AR structure, of Table A, Table B, Table C or Table D, optionally varied by a root mean square deviation of residue backbone atoms of not more than 1.235 Å, or selected coordinates thereof, and
(d) structure factor data derivable from the coordinates of (a), (b) or (c).

For example the computer system may comprise: (i) a computer-readable data storage medium comprising data storage material encoded with the computer-readable data; (ii) a working memory for storing instructions for processing said computer-readable data; and (iii) a central-processing unit coupled to said working memory and to said computer-readable data storage medium for processing said computer-readable data and thereby generating structures and/or performing rational drug design. The computer system may further comprise a display coupled to the central-processing unit for displaying structural representations.

The invention also provides such systems containing atomic coordinate data of target proteins of unknown structure wherein such data has been generated according to the methods of the invention described herein based on the starting data provided in Table A, Table B, Table C or Table D optionally varied by a root mean square deviation of residue backbone atoms of not more than 1.235 Å, or selected coordinates thereof.

Such data is useful for a number of purposes, including the generation of structures to analyse the mechanisms of action of binding partners and/or to perform rational drug design of binding partners which interact with β1-ARs, such as compounds which are agonists or antagonists.

A tenth aspect of the invention provides a computer-readable storage medium, comprising a data storage material encoded with computer readable data, wherein the data comprises one or more of:
(a) the coordinates of the turkey β1-AR structure of Table A, Table B, Table C or Table D, optionally varied by a root mean square deviation of residue backbone atoms of not more than 1.235 Å, or selected coordinates thereof;
(b) the coordinates of a target β1-AR homologue or analogue generated by homology modelling of the target based on the data in (a);
(c) the coordinates of a binding partner generated by interpreting X-ray crystallographic data or NMR data by reference to the coordinates of the turkey β1-AR structure of Table A, Table B, Table C or Table D, optionally varied by a root mean square deviation of residue backbone atoms of not more than 1.235 Å, or selected coordinates thereof, and
(d) structure factor data derivable from the coordinates of (a), (b) or (C).

The invention also includes a computer-readable storage medium comprising a data storage material encoded with a first set of computer-readable data comprising a Fourier transform of at least a portion of the structural coordinates of turkey β1-AR, of Table A, Table B, Table C or Table D, optionally varied by a root mean square deviation of residue backbone atoms of not more than 1.235 Å, or selected coordinates thereof; which data, when combined with a second set of machine readable data comprising an X-ray diffraction pattern of a molecule or molecular complex of unknown structure e.g. a target protein of unknown structure, using a machine programmed with the instructions for using said first set of data and said second set of data, can determine at least a portion of the structure coordinates corresponding to the second set of machine readable data.

The invention also provides a computer-readable data storage medium comprising a data storage material encoded with a first set of computer-readable data comprising the structural coordinates of turkey β1-AR, of Table A, Table B, Table C or Table D, optionally varied by a root mean square deviation of residue backbone atoms of not more than 1.235 Å, or selected coordinates thereof; which, when combined with a second set of machine readable data comprising an X-ray diffraction pattern of a molecule or molecular complex of unknown structure, e.g. a target protein of unknown structure, using a machine programmed with the instructions for using said first set of data and said second set of data, can determine at least a portion of the electron density corresponding to the second set of machine readable data.

It will be appreciated the that the computer-readable storage media of the invention may comprise a data storage material encoded with any of the data generated by carrying out any of the methods of the invention relating to structure solution and selection/design of binding partners to β1-AR and drug design.

The invention also includes a method of preparing the computer-readable storage media of the invention comprising encoding a data storage material with the computer-readable-data.

As used herein, “computer readable media” refers to any medium or media, which can be read and accessed directly by a computer. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

By providing such computer readable media, the atomic coordinate data of the invention can be routinely accessed to model β1-AR or selected coordinates thereof.

For example, RASMOL (Sayle et al., TIBS, Vol. 20, (1995), 374) is a publicly available computer software package, which allows access and analysis of atomic coordinate data for structure determination and/or rational drug design.

As used herein, “a computer system” refers to the hardware means, software means and data storage means used to analyse the atomic coordinate data of the invention. The minimum hardware means of the computer-based systems of the present invention comprises a central processing unit (CPU), input means, output means and data storage means. Desirably a monitor is provided to visualize structure data. The data storage means may be RAM or means for accessing computer readable media of the invention. Examples of such systems are microcomputer workstations available from Silicon Graphics Incorporated and Sun Microsystems running Unix based, Windows XP or IBM OS/2 operating systems.

An eleventh aspect of the invention provides a method for providing data for generating three dimensional structural representations of β1-AR, β1-AR homologues or analogues, complexes of β1-AR with binding partners, or complexes of β1-AR homologues or analogues with binding partners, or, for analysing or optimising binding of binding partners to said β1-AR or homologues or analogues, or complexes thereof, the method comprising:
(i) establishing communication with a remote device containing computer-readable data comprising at least one of:
   (a) the coordinates of the turkey β1-AR structure of Table A, Table B, Table C or Table D, optionally varied by a root mean square deviation of residue backbone atoms of not more than 1.235 Å, or selected coordinates thereof;
   (b) the coordinates of a target β1-AR homologue or analogue generated by homology modelling of the target based on the data in (a);
   (c) the coordinates of a binding partner generated by interpreting X-ray crystallographic data or NMR data by reference to the coordinates of the turkey β1-AR structure of Table A, Table B, Table C or Table D, optionally varied by a root mean square deviation of residue backbone atoms of not more than 1.235 Å, or selected coordinates thereof, and
   (d) structure factor data derivable from the coordinates of (a), (b) or (c); and
(ii) receiving said computer-readable data from said remote device.

The computer-readable data received from said remote device, particularly when in the form of the coordinates of the turkey β1-AR structure of Table A, Table B, Table C or Table D, optionally varied by a root mean square deviation of residue backbone atoms of not more than 1.235 Å, or selected coordinates thereof, may be used in the methods of the invention described herein, e.g. for the analysis of a binding partner structure with a β1-AR structure.

Thus the remote device may comprise e.g. a computer system or computer readable media of one of the previous aspects of the invention. The device may be in a different country or jurisdiction from where the computer-readable data is received.

The communication may be via the internet, intranet, e-mail etc, transmitted through wires or by wireless means such as by terrestrial radio or by satellite. Typically the communication will be electronic in nature, but some or all of the communication pathway may be optical, for example, over optical fibers.

A twelfth aspect of the invention provides a method of obtaining a three dimensional structural representation of a crystal of a turkey β1-AR, which method comprises providing the coordinates of the turkey β1-AR structure of Table A, Table B, Table C or Table D, optionally varied by a root mean square deviation of residue backbone atoms of not more than 1.235 Å, or selected coordinates thereof, and generating a three-dimensional structural representation of said coordinates.

For example, the structural representation may be a physical representation or a computer generated representation. Examples of representations are described above and include, for example, any of a wire-frame model, a chicken-wire model, a ball-and-stick model, a space-filling model, a stick model, a ribbon model, a snake model, an arrow and cylinder model, an electron density map or a molecular surface model.

Computer representations can be generated or displayed by commercially available software programs including for example QUANTA (Accelrys .COPYRIGHT. 2001, 2002), O (Jones et al., Acta Crystallogr. A47, pp. 110-119 (1991)) and RIBBONS (Carson, J. Appl. Crystallogr., 24, pp. 9589-961 (1991)).

Typically, the computer used to generate the representation comprises (i) a computer-readable data storage medium comprising a data storage material encoded with computer-readable data, wherein said data comprise the coordinates of the turkey β1-AR structure; of Table A, Table B, Table C or Table D, optionally varied by a root mean square deviation of residue backbone atoms of not more than 1.235 Å, or selected coordinates thereof; and (ii) instructions for processing the computer-readable data into a three-dimensional structural representation. The computer may further comprise a display for displaying said three-dimensional representation.

A thirteenth aspect of the invention provides a method of predicting one or more sites of interaction of a β1-AR or a homologue thereof, the method comprising: providing the coordinates of the turkey β1-AR structure of Table A, Table B, Table C or Table D, optionally varied by a root mean square deviation of residue backbone atoms of not more than 1.235 Å, or selected coordinates thereof; and analysing said coordinates to predict one or more sites of interaction.

For example, a binding region of a β1-AR for a particular binding partner can be predicted by modelling where the structure of the binding partner is known. Typically, the fitting and docking methods described above would be used. This method may be used, for example, to predict the site of interaction of a G protein of known structure as described in viz Gray J J (2006) Curr Op Struc Biol Vol 16, pp 183-193.

A fourteenth aspect of the invention provides a method for assessing the activation state of a structure for β1-AR, comprising: providing the coordinates of the turkey β1-AR structure, of Table A, Table B, Table C or Table D, optionally varied by a root mean square deviation of residue backbone atoms of not more than 1.235 Å, or selected coordinates thereof; performing a statistical and/or topological analysis of the coordinates; and comparing the results of the analysis with the results of an analysis of coordinates of proteins of known activation states.

For example, protein structures may be compared for similarity by statistical and/or topological analyses (suitable analyses are known in the art and include, for example those described in Grindley et al (1993) J Mol Biol Vol 229: 707-721 and Holm & Sander (1997) Nucl Acids Res Vol 25: 231-234). Highly similar scores would indicate a shared conformational and therefore functional state eg the inactive antagonist state in this case.

One example of statistical analysis is multivariate analysis which is well known in the art and can be done using techniques including principal components analysis, hierarchical cluster analysis, genetic algorithms and neural networks.

By performing a multivariate analysis of the coordinate data of the turkey β1-AR structure of Table A, Table B, Table C or Table D, optionally varied by a root mean square deviation of residue backbone atoms of not more than 1.235 Å or selected coordinates thereof, and comparing the result of the analysis with the results of the analysis performed on coordinates of proteins with known activation states, it is possible to determine the activation state of the coordinate set analysed. For example, the activation state may be classified as ‘active’ or ‘inactive’.

A fifteenth aspect of the invention provides a method of producing a protein with a binding region that has substrate specificity substantially identical to that of β1-AR, the method comprising
a) aligning the amino acid sequence of a target protein with the amino acid sequence of a β1-AR;
b) identifying the amino acid residues in the target protein that correspond to any one or more of the following positions according to the numbering of the turkey β1-AR as set out in FIG. 6: 117, 118, 121, 122, 125, 201, 203, 207, 211, 215, 306, 307, 310 and 329; and
c) making one or more mutations in the amino acid sequence of the target protein to replace one or more identified amino acid residues with the corresponding residue in the turkey β1-AR.

By “an amino acid residue that corresponds to” we include an amino acid residue that aligns to the given amino acid residue in turkey β1-AR when the turkey β1-AR and target protein are aligned using e.g. MacVector and CLUSTALW.

For example, amino acid residues contributing to the ligand binding site of β1-AR include amino acid residues 117, 118, 121, 122, 125, 201, 203, 207, 211, 215, 306, 307, 310 and 329. Thus a binding site of a particular protein may be engineered using well known molecular biology techniques to contain any one or more of these residues to give it the same substrate specificity. This technique is well known in the art and is described in, for example, Ikuta et al (J Biol Chem (2001) 276, 27548-27554) where the authors modified the active site of cdk2, for which they could obtain structural data, to resemble that of cdk4, for which no X-ray structure was available.

Preferably, all 14 amino acids in the target portion which correspond to amino acid residues 117, 118, 121, 122, 125, 201, 203, 207, 211, 215, 306, 307, 310 and 329 of the turkey β1-AR are, if different, replaced. However, it will be appreciated that only 13, 12, 11, 10, 9, 8, 7, 6, 5, 4, 3, 2 or 1 amino acid residues may be replaced.

Preferences for the target protein are as defined above with respect to the first aspect of the invention.

A sixteenth aspect of the invention provides a method of predicting the location of internal and/or external parts of the structure of β1-AR or a homologue thereof, the method comprising: providing the coordinates of the turkey β1-AR structure of Table A, Table B, Table C or Table D, optionally varied by a root mean square deviation of residue backbone atoms of not more than 1.235 Å, or selected coordinates thereof and analysing said coordinates to predict the location of internal and/or external parts of the structure.

For example, from the three dimensional representation, it is possible to read off external parts of the structure, eg surface residues, as well as internal parts, eg residues within the protein core. It will be appreciated that the identification of external protein sequences will be especially useful in the generation of antibodies against a β1-AR.

A seventeenth aspect of the invention provides a peptide of not more than 100 amino acid residues in length comprising at least five contiguous amino acid residues which define an external structural moiety of the β1-AR.

Examples of suitable external structural moieties include the six surface loops of contiguous residues and the three surface (non-transmembrane) helices as follows:
CL1 Residues 68-76
EL1 Residues 99-116
CL2 (short surface helix) Residues 143-145
EL2 (short surface helix) Residues 174-207
EL3 Residues 311-319
H8 (short surface helix) Residues 341-358

Thus in one embodiment, the peptide of not more than 100 amino acid residues comprises at least five contiguous amino acid residues from any of the external structural moieties defined above. It will be appreciated that the peptide may comprise at least five contiguous amino acid residues from one external structural moiety defined above and five contiguous amino acid residues from one or more different external structural moieties defined above.

It will be appreciated that such peptides may serve as epitopes for the generation of binding partners, e.g. antibodies against a β1-AR. Thus, the invention also includes a binding partner selected to bind to the peptide of the eighteenth aspect of the invention.

The crystallisation of the turkey β1-AR has led to many interesting observations about its structure, including its ligand binding site. Thus it will be appreciated that the invention allows for the generation of mutant β1-ARs wherein residues corresponding to these areas of interest are mutated to determine their effect on β1-AR function and ligand binding specificity.

Accordingly, an eighteenth aspect of the invention provides a mutant β1-AR, wherein the β1-AR before mutation has a binding region in the position equivalent to the binding region of turkey β1-AR that is defined by residues including 117, 118, 121, 122, 125, 201, 203, 207, 211, 215, 306, 307, 310 and 329 of β1-AR according to the numbering of the turkey β1-AR as set out in FIG. 6 and wherein one or more residues equivalent to 117, 118, 121, 122, 125, 201, 203, 207, 211, 215, 306, 307, 310 and 329 forming part of the binding region of β1-AR is mutated.

Residues in proteins can be mutated using standard molecular biology techniques as are well known in the art.

A nineteenth aspect of the invention provides a method of making a β1-AR crystal comprising: providing purified β1-AR; and crystallising the β1-AR either by using the sitting drop or hanging drop vapour diffusion technique, using a precipitant solution comprising 0.1M ADA (N-(2-acetamido) iminodiacetic acid) (pH5.6-9.5). and 25-35% PEG 600.

In a preferred embodiment, the precipitant solution comprises 0.1M ADA (pH 6.9-7.3) and 29-32% PEG600. However, it will be appreciated that any other buffer at a concentration between 0.03 and 0.30 M may be used, and that any PEG from PEG400 to PEG5000 may be used.

A twentieth aspect of the invention provides a crystal of β1-AR having the structure defined by the coordinates of the turkey β1-AR structure of Table A, Table B, Table C or Table D, optionally varied by a root mean square deviation of residue backbone atoms of not more than 1.235 Å, or selected coordinates thereof. Typically, the crystal has a resolution of 2.7 Å or better.

The space group of the crystal may be either P1 or C2.

Thus, in one embodiment the crystal has P1 symmetry with unit cell dimensions a=55.5 Å±1 Å, b=86.8 Å±, 20 Å, c=95.50 Å±20 Å.

In another embodiment, the crystal has C2 symmetry with unit cell dimensions a=145-195 Å±20 Å, b=55.5 Å±1 Å, c=85-120 Å.

The invention also includes a co-crystal of β1-AR having the structure defined by the coordinates of the turkey β1-AR structure of Table A, Table B, Table C or Table D, optionally varied by a root mean square deviation of residue backbone atoms of not more than 1.235 Å, or selected coordinates thereof, and a binding partner. Typically, the crystal has a resolution of 2.7 Å or better.

The invention includes the use of the coordinates of the turkey β1-AR structure of Table A, Table B, Table C or Table D, optionally varied by a root mean square deviation of residue backbone atoms of not more than 1.235 Å, or selected coordinates thereof to solve the structure of target proteins of unknown structure.

The invention includes the use of the coordinates of the turkey β1-AR structure of Table A, Table B, Table C or Table D, optionally varied by a root mean square deviation of residue backbone atoms of not more than 1.235 Å, or selected coordinates thereof to identify binding partners of a β1-AR.

The invention includes the use of the coordinates of the turkey β1-AR structure of Table A, Table B, Table C or Table D, optionally varied by a root mean square deviation of residue backbone atoms of not more than 1.235 Å, or selected coordinates thereof in methods of drug design where the drugs are aimed at modifying the activity of the β1-AR.

The invention will now be described in more detail with respect to the following Figures and Examples wherein:

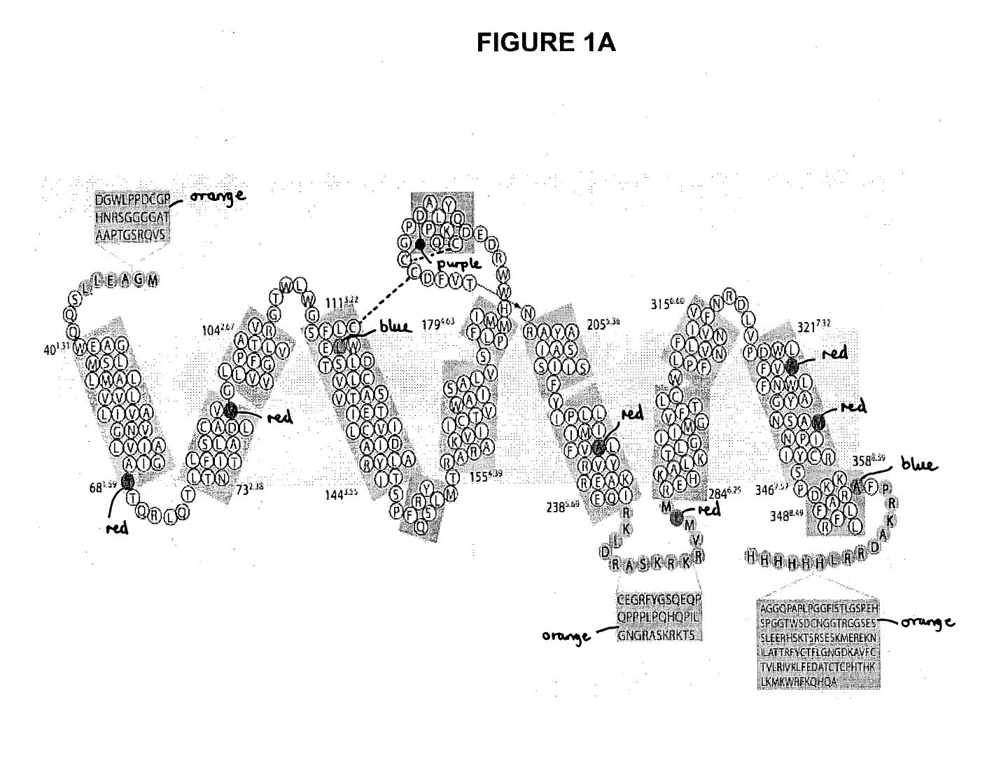

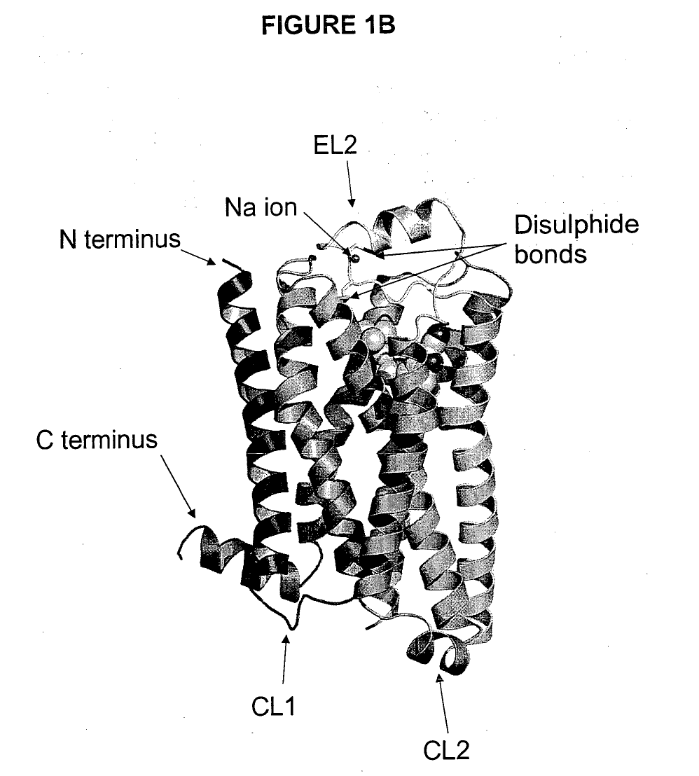

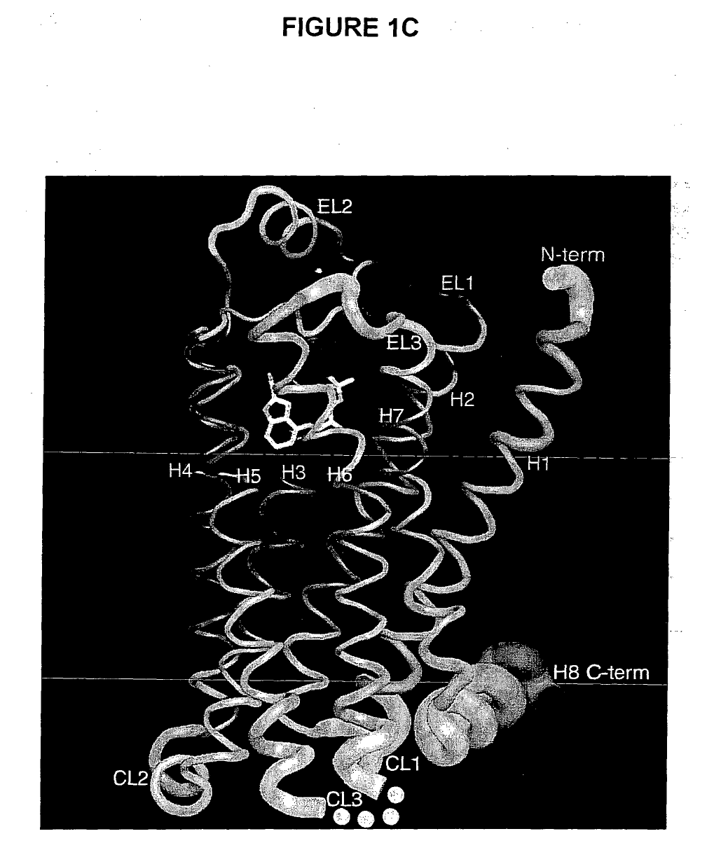

FIG. 1 (A) Schematic diagram of the β1 sequence in relation to secondary structure elements. Amino sequence in white circles indicates regions that are well ordered, but sequences in a grey circle were not resolved in the structure. Grey sequences on an orange background were deleted to make the β1 construct for expression. Thermostabilising mutations are in red and two other mutations C116L and C358A are in blue. The Na⁺ is in purple and the two disulphide bonds are depicted as dotted lines. Numbers refer to the first amino acid residue in each helix, with the Ballesteros-Weinstein numbering in superscript. (B) Ribbon representation of the β1 structure. The N-terminus, C-terminus, the Na⁺ ion, the two disulphide bonds extracellular loop 2 (EL2) and intracellular loops 1 and 2 (CL1, CL2) are labelled (C) B factors depicted on a ribbon representation of the β1 configuration. Order of B factors from low to high is EW, H4, H3, H7, H5, H2, H6, EL2, CL2, H1, EL3, CL3, CL1, N-term and H8C-term.

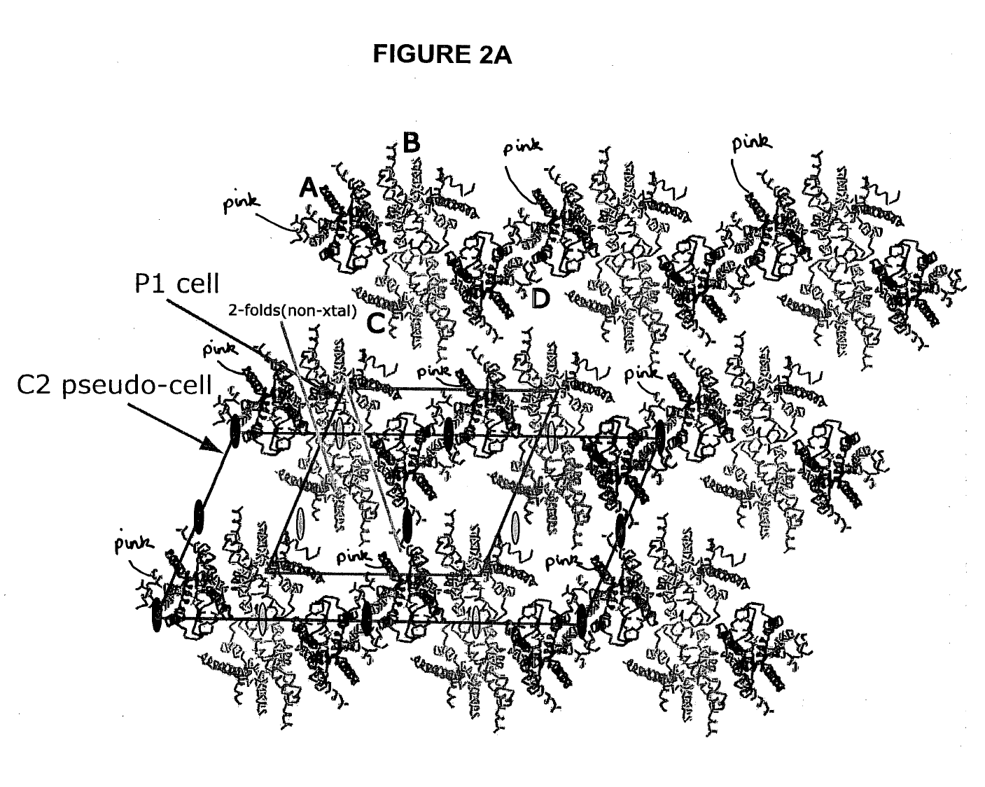

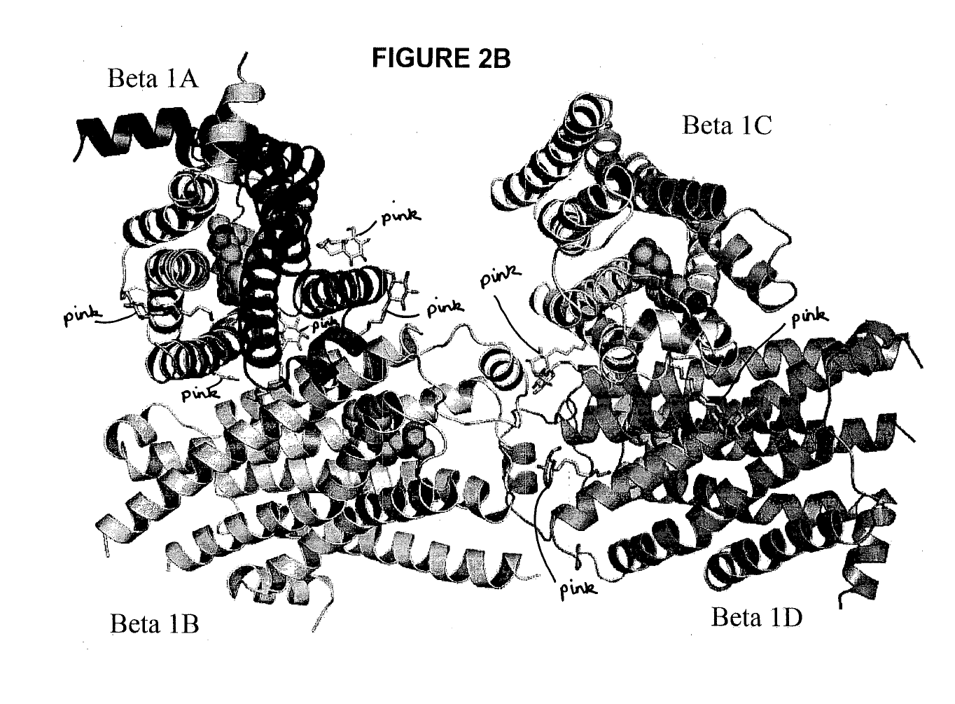

FIG. 2 (A) Packing of the β1 molecules in the C2 and P1 crystals obtained, showing how the packing is related. (B) Ribbon representation of the molecules within one unit cell of the P1 crystal form. Octylthiomaltoside detergent molecules, which pack at the interfaces between the receptors, are shown in pink.

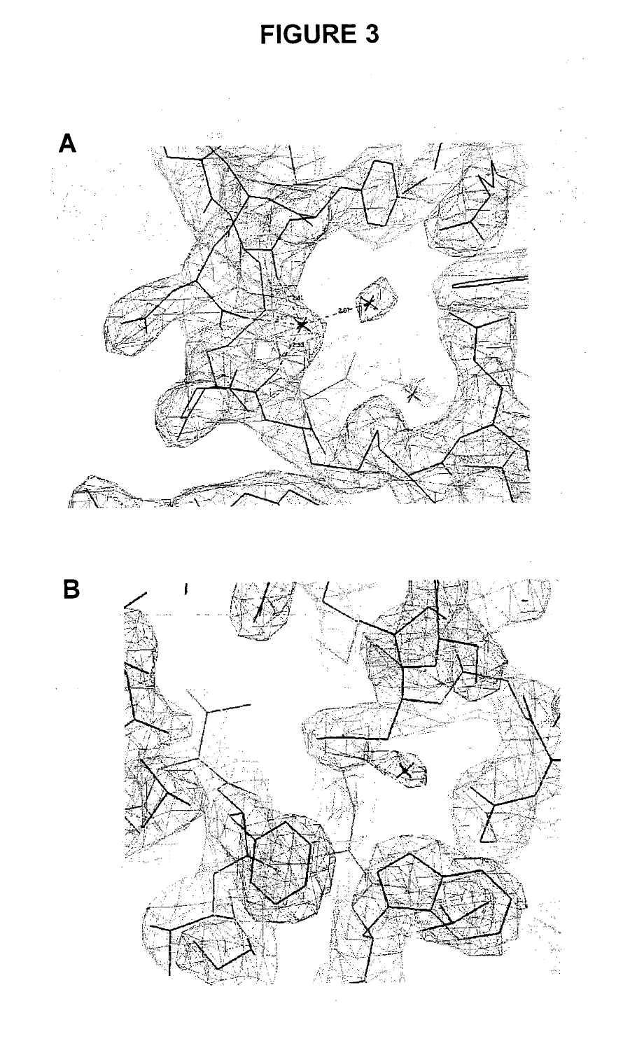

FIG. 3 Representative regions of electron density in the structure. (A) Co-ordination of the Na⁺ by the backbone carbonyl groups from amino acid residues Cys192, Asp195, Cys198 and a water molecule. (B) Water molecule hydrogen bonded to Trp303 in helix 6.

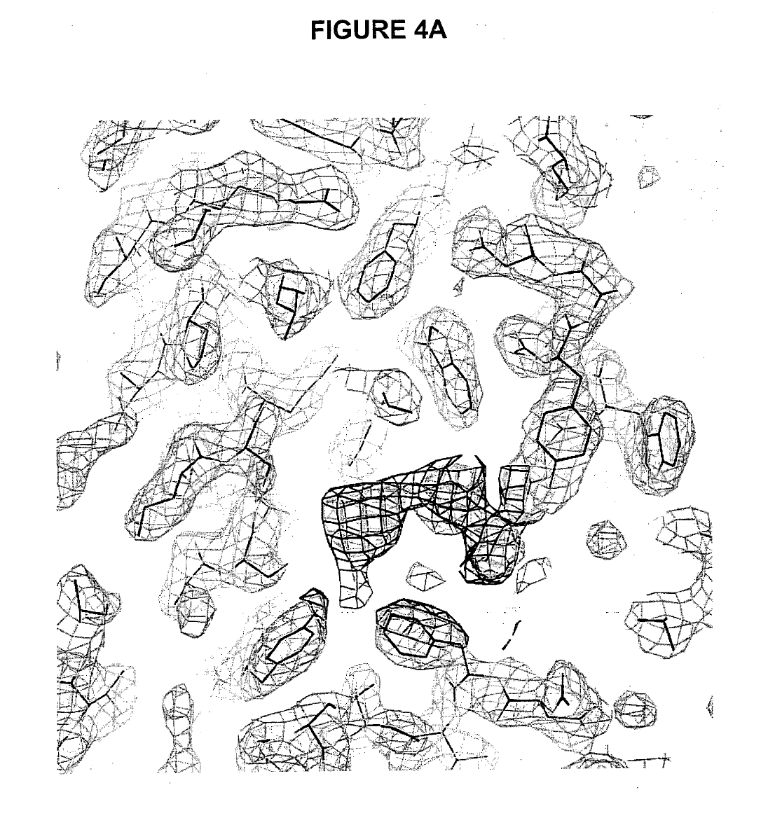

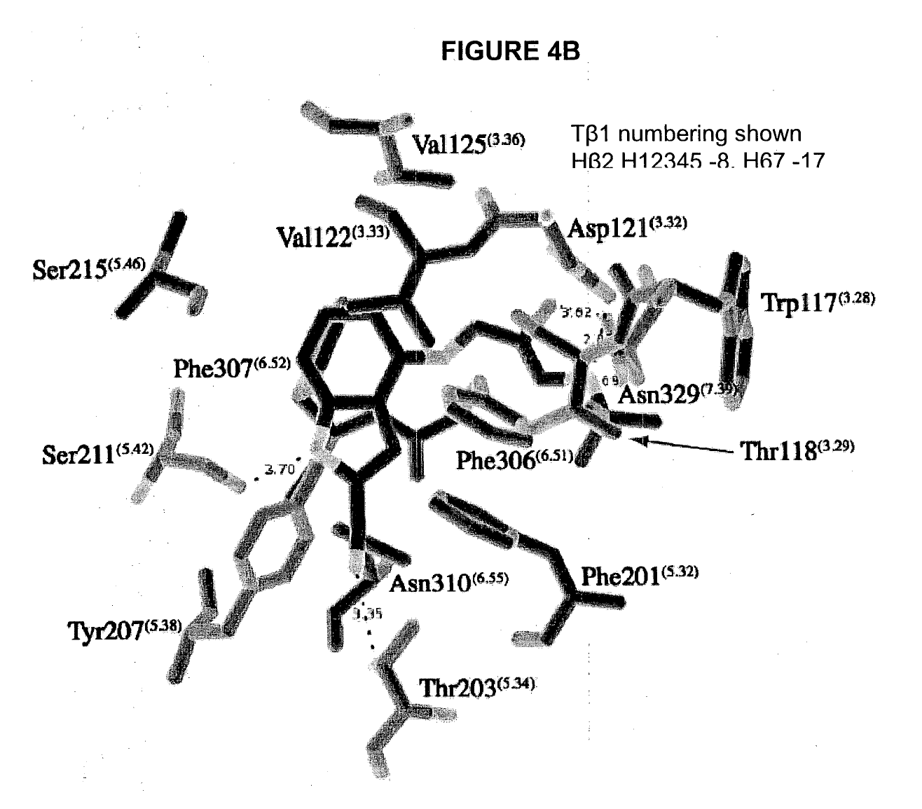

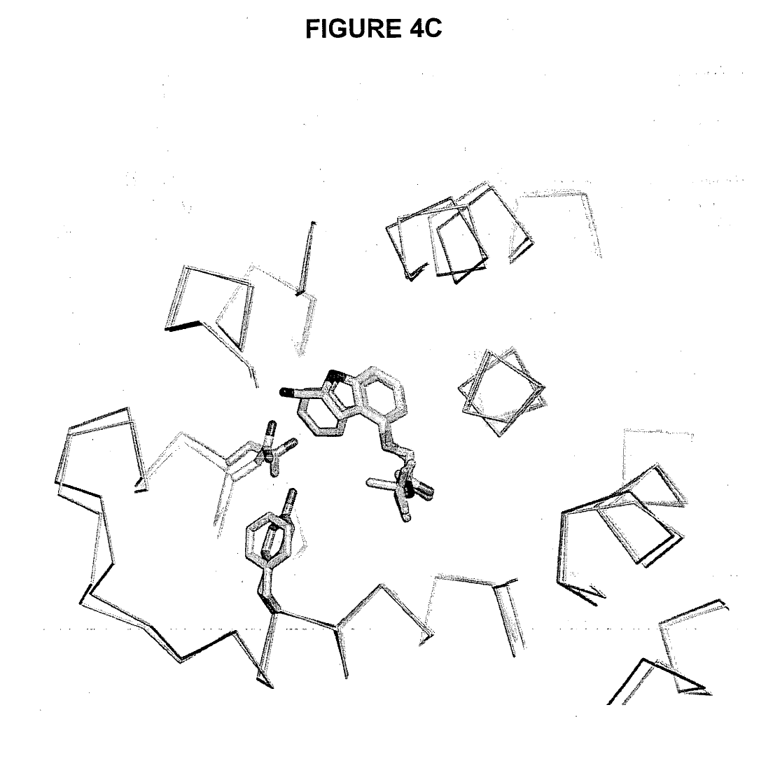

FIG. 4 The ligand binding region. (A) 2Fo-Fc omit map showing the unrefined density for cyanopindolol after molecular replacement using only the peptide co-ordinates form human β2 receptor. (B) and (C) Position of amino acid residues that interact with the ligand cyanopindolol.

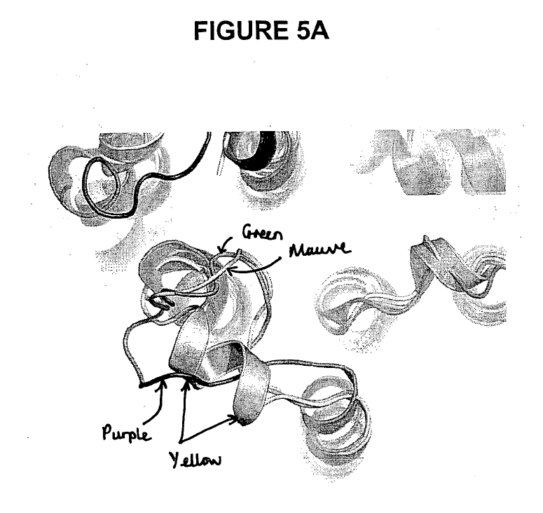

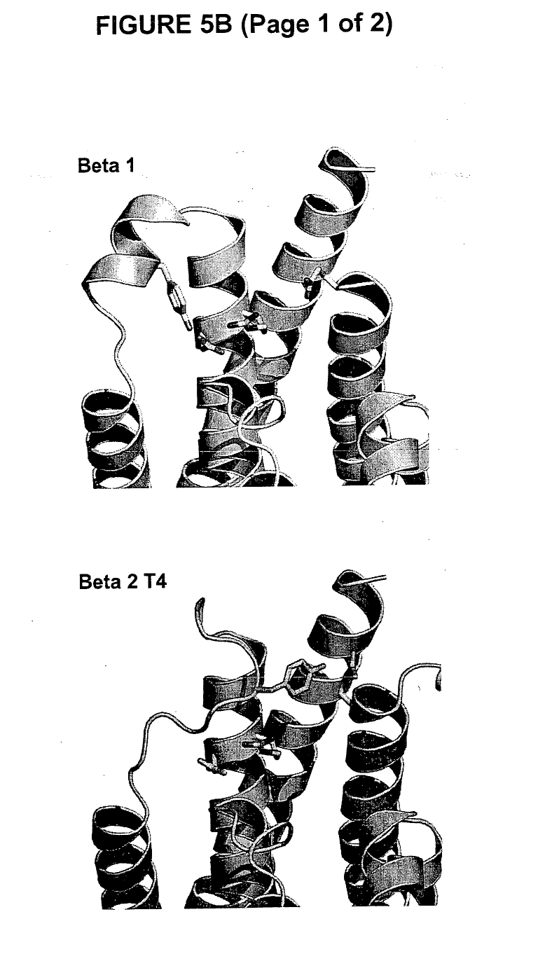

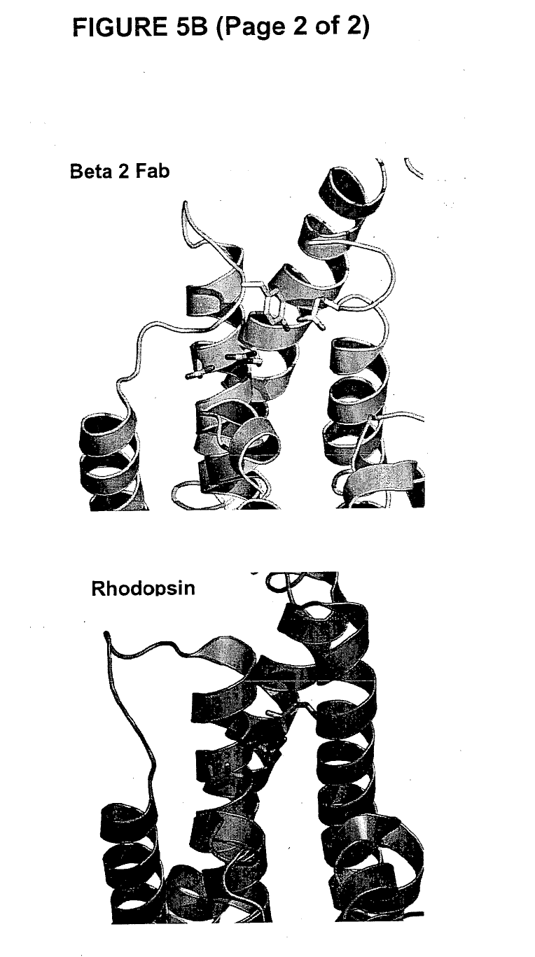

FIG. 5 (A) Comparison of the CL2 loop region between the b1 structure (yellow), the β2-T4 lysozyme fusion (green), the β2-Fab complex (mauve) and rhodopsin (purple). (B) Comparison of the ionic regions in β1, rhodopsin and the two β2 structures. The amino acid residues shown in the β1 structure are Tyr149^{3.60}, Asp138^{3.49}, Arg139^{3.50} and Glu285^{6.29}.

FIG. 6 Alignment of the turkey β-adrenergic receptor with human β1, β2 and β3 receptors.

FIG. 7 Multiple sequence alignment of turkey β1-AR (beta 36/m23 construct) with (1) β2-AR T4 lysozyme fusion protein (structure of which is described in Cherezov et al (2007) and Rosenbaum et al (2007)) and (2) β2-AR (β2-AR 365 construct, structure of which is described in Rasmussen et al (2007)).

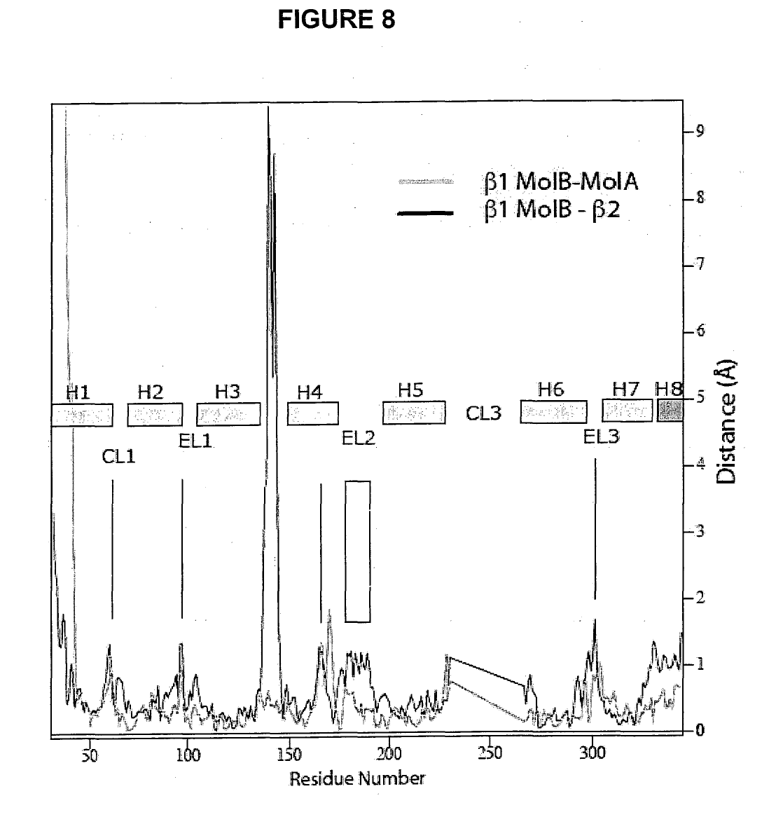

FIG. 8 Distances between corresponding Cα atoms after superposition of β1-AR-m23 and the human β2-AR (PDB no: 2RH1) compared with superposition of molecules A and B of β1-AR-m23.

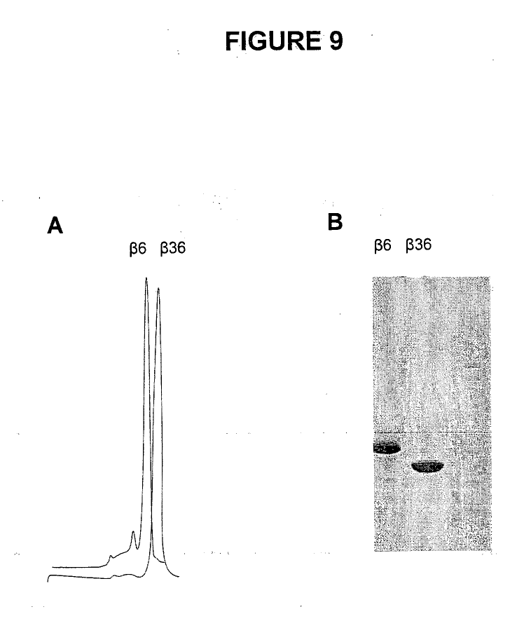

FIG. 9 (A) Size exclusion elution profiles of Beta 6 and Beta 36 (B) SDS PAGE of Beta 6 and Beta 36

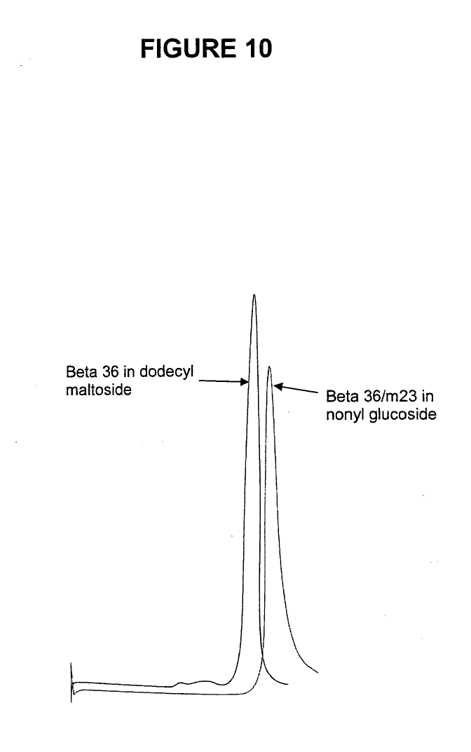

FIG. 10 Size-exclusion profiles of Beta 36 in dodecylmaltoside (left peak, eluted earlier), and Beta 36/m23 in nonylglucoside (right peak, eluted later).

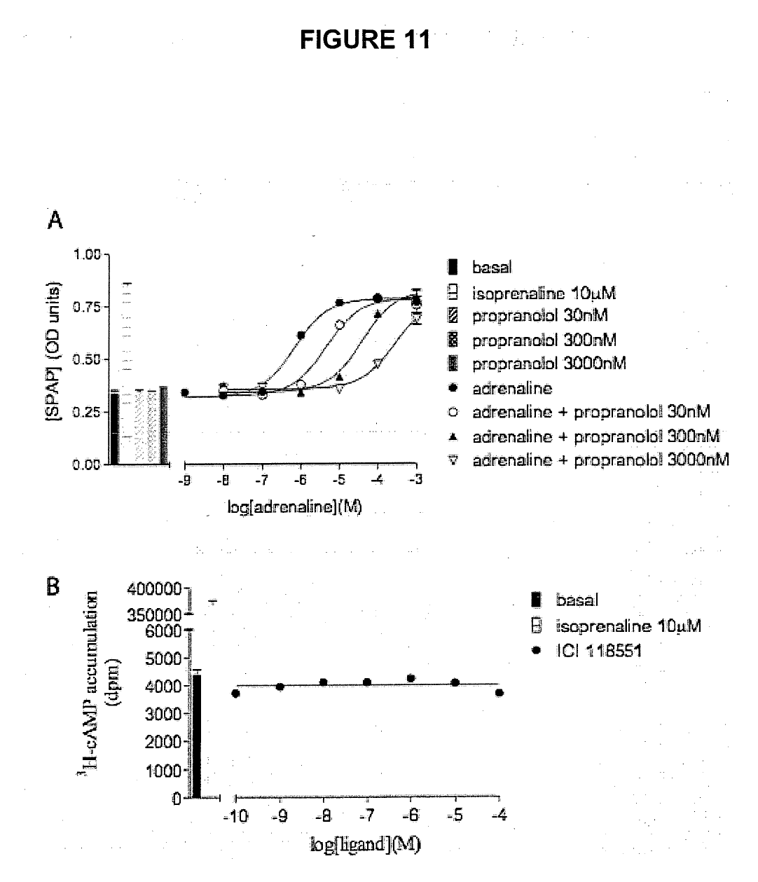

FIG. 11 Activation of G-proteins by m23 mutant receptor as measured by ATP binding as a function of adrenaline concentration and its inhibition by antagonist propranolol. This demonstrates that the inverse agonist ICI118551 does not depress the cAMP accumulation. Both panels show the pharmacological behaviour of m23.

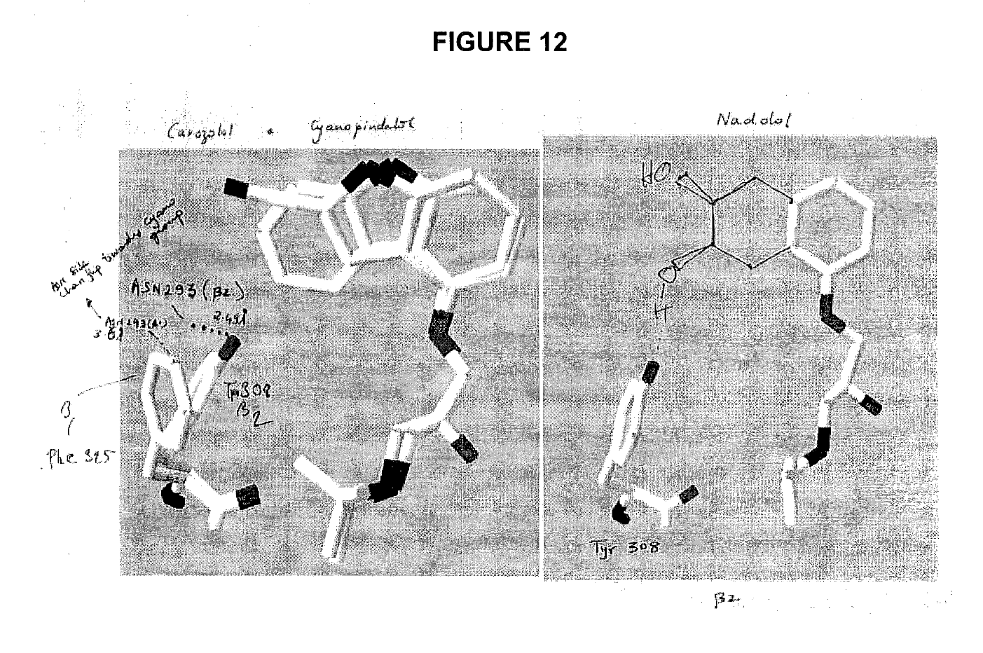

FIG. 12 Relationship between cyanopindolol in beta1 and carazolol in beta2 and the residues Phe325 in beta1 and Tyr308 in beta2, together with one possible interaction which might occur between hydroxyl groups of ceratin sub-type specific ligands and the hydroxyl group of Tyr308 in beta2.

### EXAMPLE 1

### Structure Determination of Turkey β1-AR

### Introduction

The G protein coupled receptor superfamily has a major role in transmembrane signal transduction in organisms from yeast to man and many are important biomedical drug targets. We report the 2.7 Å resolution crystal structure of a β1 adrenergic receptor (b1AR), whose conformation and improved thermostability have been selected by systematic mutagenesis and binding to the antagonist, cyanopindolol. The receptor mutant, b1AR-m23, is in an inactive conformation and there is no ionic lock present between helix 3 and 7. The interactions of cyanopindolol with the β1 receptor are similar to those of carazolol with β2AR, though some small significant differences help to understand important aspects of the selectivity between β1 and β2 antagonists. There is a well-defined helix in cytoplasmic loop 2, absent in the b2 structures, which directly links this region to which G proteins bind upon agonist binding to the highly conserved DRY motif at the end of helix 3 essential for receptor activation.

### Results and Discussion

### Crystallisation of the β1 Adrenergic Receptor

There are two major prerequisites to the crystallisation of any membrane protein, once the problems of overexpression and purification have been overcome. Firstly, the protein must be sufficiently stable in detergent solution for crystals to form and, secondly, the protein must exist primarily in a single conformational state. GPCR crystallisation is therefore extremely challenging, because they are usually unstable in detergent and spontaneously cycle between an inactive antagonised state (R) and an active agonist-bound state (R*), even in the absence of ligands. Both recent structures of β2 required the receptor to be bound to the partial inverse agonist carazolol, so that the receptors were all in a single antagonised (R) conformation. The human β2 receptor was sufficiently stable to purify in mild detergents such as DDM, but crystals were only obtained either when β2 was bound to a specific F_{ab} fragment from a conformationally neutral monoclonal antibody (Day et al (2007) Nat Methods 4(11): 927-9) or by the selection of a protease-resistant T4 lysozyme fusion (Rosenbaum et al., 2007); in both cases the additional proteins made essential lattice contacts within the crystals, and in the T4 fusion induced constitutive activation. Stabilisation of the receptor during crystallisation was either achieved by the formation of detergent-lipid bicelles (DMPC/CHAPSO) around the protein (Rasmussen et al, 2007) or by the use of cholesterol-doped lipidic cubic phases (Cherezov et al, 2007).

The human β1 receptor has proven more difficult to purify than β2, because it is unstable once solubilised in detergent, so we therefore used the turkey β1 receptor which is considerably more stable than its human homologue (Parker & Ross). Short-chain detergents, such as nonyl- and octyl-glucosides, are the best choice for crystallisation of small membrane proteins, but β1 was unstable in them and precipitated upon detergent exchange (Warne et al 2003). We therefore expressed β1 in an Escherichia coli expression system (Grisshammer et al) and evolved it into a conformationally thermostabilised form (β1-m23) that is stable even in short-chain detergents (Serrano PNAS). The six point mutations in β1-m23 not only increased the thermostability of the receptor in dodecylmaltoside (DDM) by 21° C., but also altered the equilibrium between R and R* so that the mutant receptor was preferentially in the antagonised (R) state (Serrano-Vega et al 2008). The receptor construct that crystallised (FIG. 1) has deletions at the N-terminus, C-terminus and in cytoplasmic loop 3 to remove regions that were predicted to be unstructured (Warne et al 2003). It also contains 8 point mutations, 6 for thermostabilisation (R68^{1.59}S, M90^{2.52}V, Y227^{5.58}A, A282^{6.27}L, F327^{7.38}A, F338^{7.49}M), one for improved expression (C116^{3.27}L) and one for the removal of a palmitoylation site (C358^{8.53}A).

### Pharmacological Analysis of β1-m23

In any crystallographic study it is essential to define exactly what conformational state the receptor is in to understand how function relates to structure. In a pharmacological analysis, the mutant receptor β1-m23 bound the antagonists dihydroalprenolol and cyanopindolol with similar affinities to the wild-type receptor, but the agonists noradrenaline and isoprenaline bound more weakly by a factor of 2470 and 650 respectively (Serrano-Vega et a/). This reflects a change in the preferentially adopted global conformation of the receptor to an antagonised state. The structure we have determined contains cyanopindolol in the binding region; it is known that cyanopindolol binds to β1-m23 with very high affinity (60 pM) and that it is an antagonist. Thus the structure determined is that of β1 in the antagonised (inverse agonist) conformation.

### Overall Structure of the β1 Receptor

Crystals of β1-m23 were obtained in octylthioglucoside after an extensive crystallisation screen. Two closely related crystal forms with either C2 or P1 symmetry were observed; the packing is very similar in both space groups, with 4 molecules in the P1 unit cell and 8 in the C2 cell, which has one axis twice as large as the comparable axis in the P1 cell. The pairs of molecules related by noncrystallographic symmetry in C2 are slightly rotated to give the P1 form (FIG. 2) The C2 crystals diffracted anisotropically with diffraction limits varying between 2.6-3.5 Å, whereas the P1 crystals showed isotropic diffraction to beyond 2.7 Å. The β1 structure was solved to 2.7 Å (Table 1) by molecular replacement. The four receptor molecules (A-D) were independently refined, and thus allow four different views of the same molecule. Molecules B and C are similar to each other (rmsd 0.18 Å for 273 residues) and molecules A and D are also similar to each other (rmsd 0.22 Å for 273 residues); molecules A and D both differ from molecules B and C by an average rmsd of 0.48 Å. The major difference between molecules A & D and B & C (which was excluded from the above comparison) is that there is outward kink of the 12 N-terminal residues of helix 1 (Trp40-Val51) by about 60°, which accommodates molecules A & D within the crystal lattice: the helix boundaries and overall structural motifs are presented in FIG. 1. There is well-defined density for all the transmembrane helices, extracellular loops (1-3), two intracellular loops (CL1 & 2) and helix 8 (except in molecule C). There was no density corresponding to most of CL3 due to disorder. Included in the structure are well-ordered detergent molecules of octylthioglucoside that sometimes make essential contacts between neighbouring receptor molecules. In addition, there is one Na⁺ ion per receptor and 5-9 well-defined water molecules (FIG. 3) per receptor. Unless otherwise stated, all further discussion refers to molecule B, as only this molecule has an unkinked helix 1 and includes helix 8.

The amino acid sequence of the turkey β1 receptor is 65% identical to that of the human β2 receptor over residues 39-358 excluding CL3 residues 238-285 i.e. excluding the N- and C-termini and CL3) and it is therefore unsurprising that the structure of the transmembrane regions of β1 and β2 are very similar. The best superposition of the β2 (2rh1) and β1 (chain B) structure is based on selected residues in helices 3,5,6,7, as these helices form most of the ligand binding region; 78 alpha carbons can be superimposed with an rmsd of 0.25 Å. The rmsd over all the transmembrane helices is 0.4 Å for backbone (C-α, C, N atoms). In addition, the structure of the three extracellular loops in β1AR are very similar to β2AR with an overall rms deviation of 0.83 Å for backbone atoms (C-α, C, N in extracellular loops), which is consistent with high sequence conservation of these regions in the DAR family (FIG. 6). On the extracellular surface, there is a sodium ion co-ordinated by the carbonyl groups in the peptide backbone from residues Cys192, Asp195, Cys198 and one water molecule. The sodium ion was assigned based upon its coordination geometry and its presence at the negative end of the EL2 α-helix dipole is in a position often favoured by positive ions or ligands.

Overall, 27 water molecules were built into the map (Table 2) using the criteria that spherical densities must be >1.0σ in the 2Fo-Fc difference map and they must form at least two H-bonds with good geometry. Only one water molecule was likely to be important structurally as it maintains the structure of the kink in helix 6 and H-bonds to W303, which is thought to be important in the light-activation of rhodopsin. All other waters tended to be less buried, and none are absolutely conserved between β1 and β2, or even between the different molecules of β1 in the same unit cell. Other water molecules must be present throughout the core of the β1 structure to, solvate polar amino acid residues, but they must be only partially ordered and are therefore unlikely to have a strong influence on substrate specificity, although they could affect the overall stability of each state of the receptor, as well as the equilibrium between R and R*.

The 6 point mutations that thermostabilised β1 were essential for obtaining well-diffracting crystals (Serrano-Vega et al 2008). It is not clear, now the structure has been solved, why the mutations make β1AR-m23 more thermostable than the wild type β1 receptor. At each mutated position there were no significant changes in the Cα backbone when compared with the 62 structure and, therefore, the mutations have not distorted the structure of the receptor. This is consistent with the observations that β1AR-m23 binds antagonists with similar affinities to the wild type receptor (Serranno-Vega et al 2008) and that it can couple efficiently to G. proteins.

### Structure of the Cytoplasmic Loops

All three βAR structures have a similar conformation of CL1, but there are major differences in CL3; these differences are not of physiological relevance because they arise due to either partial deletion of the loop (β1), partial deletion and insertion of T4 lysozyme (β2-T4) or by formation of a complex with an antibody fragment (β2:Fab). However, differences in the structure of CL2 (FIG. 5) are important, because this region is highly conserved and the amino acid sequence is unchanged in each of the three βARs crystallised. In β1, CL2 forms a short α-helix whereas in both the β2 structures and in rhodopsin this region is in an extended conformation (FIG. 5). In the β2:Fab structure the second intracellular loop is in contact with the neighboring antibody fragment (Rassmusen et al 2007) and might therefore be displaced. In the human β2-T4 structure an α-helix in CL2 may not be present because of lattice contacts involving the lysozyme fusion protein and the N-terminus of CL2 (Cherezov et al, 2007).

The CL2 loop has been proposed to function as the switch enabling G protein activation (Burstein et al 1998) and, from the β1 structure, it is clear that this region also has an important contact to the adjacent highly conserved D^{3.49}R^{3.50}Y^{3.51} motif in helix 3. In rhodopsin, there is a salt bridge formed between Arg^{3.50} and Glu^{6.30}, the ionic lock, which has been proposed to play an essential role in maintaining all GPCRs in an inactive state (Ballesteros et al (2001) JBC 276, 29171-29177) but is subsequently broken upon receptor activation. In none of the adrenergic receptor structures is there an ionic interaction between the Arg139^{3.50} of the DRY motif and the Glu285^{8.30} in helix 6; as the structure of β1 is of the antagonised state, there is, therefore, no interhelical ionic lock in the inactive state of this receptor and, by implication, all βARs (FIG. 5). This is mainly due to the increased distance between the C_{α} atoms of Arg^{3.50} and Glu^{6.30} in β1 (10.9 Å) and β2 (11.2 Å) compared with rhodopsin (8.7 Å). There is, however, an intrahelical interaction between Asp^{3.49} and Arg^{3.59} of the DRY motif in all three β3AR structures. The helical conformation of CL2 in the β1 structure positions Tyr149^{3.89} sufficiently close to Asp138^{3.49} of the DRY motif to allow the formation of a H-bond. Supporting evidence for this structural role of Tyr149^{3.89} comes from the observation that the Y149A mutation makes β1AR much less thermally stable (Table 3). The equivalent Tyr141^{3.60} in both β2 structures is in a cavity between helix 3, 4 and 6, but the biological relevance of this is unclear, due to the perturbations in this region caused by either the T4 lysozyme fusion or by the bound antibody F_{ab}. Interestingly, CL2 was predicted to be α-helical based upon a mutagenic study of the m5 muscarinic receptor and the mutation Y138^{3.80}A led to increased constitutive activity in the receptor (Burstein at al 1998).

### The Ligand Binding Region and the Selectivity of β Receptor Antagonists

The β1AR was crystallised in the presence of cyanopindolol, which is similar in structure to carazolol that is present in the ligand binding region of both β2 structures; both these ligands bind with very high affinity to all β1-ARs and β2-ARs. In the β1 structure there are 14 amino acid residues whose side chains make contacts with cyanopindolol in the ligand binding region; 5 side chains are from helix 3, 3 each from helices 5 and 6, one from helix 7 and 2 from EL2. All these residues are identical to those in β2 and the mode of binding of cyanopindolol to β1 is, therefore, very similar to that of carazolol in β2. However, the extra benzene ring in carazolol, due to a van der Waals contact with Y199^{5.38}, pushes the ligand more deeply into the binding site, by 0.8 Å. The nitrogen in the cyano-moiety of cyanopindolol makes a hydrogen bond with the hydroxyl of T203^{(5.34)} which is located together with F201^{5.32} at the inner most strand of EL2 that comes close to the ligand (FIG. 4). The same H-bonds between the ligand and D121^{(3.32)}, N329^{(7.39)} and S211^{(5.42)} are present in both complexes, but the rotamer conformation of S211 is different.

Cyanopindolol and carazolol are non-specific RAR ligands, so it is unsurprising that they bind to β1 and β2 similarly. To explain why some ligands preferentially bind to either β1 or β2, there must be consistent differences in amino acid residues close to the ligand binding region to have either a direct or indirect effect on ligand binding; at the opposite extreme, there must be global changes in the binding site due to multiple differences throughout the protein domain, as illustrated in FIG. 8. Regarding the former mechanism, a comparison of residues within 8 Å of the binding region amongst all β2 and β1 receptors identified only two residues that are highly conserved but different between the two receptor families. The respective residues are Val172 and Phe325 in β1, which are equivalent to Thr164 and Tyr308 in β2; both these changes introduce polar residues into the binding region of β2 relative to β1 and, therefore, could have a profound effect upon ligand binding and selectivity, either directly or via a different distribution of water molecules. Tyr308 has also been implied by a mutagenesis study to be important for the agonist selectivity by mutagenesis (Kikkawa et al (1998) Mol Pharmacol 53: 128-134). The closest distance between cyanopindolol and the side chain of Vail 72 or Phe325 is 8 Å or 6 Å respectively. In the β2 receptor, Tyr308 is maintained closer to the binding region via a hydrogen bond to Asn293 and it is close to the carazolol heterocyclic ring, but in the β1 receptor the equivalent residue, Phe325, moves away from the binding region and the Asn310 side chain changes position to make a hydrogen bond with the cyano group of cyanopindolol; therefore there is no contact between Phe325 in β1 and cyanopindolol. The presence of Tyr308 adjacent to the carazolol heterocyclic ring and the absence of an equivalent H-bond acceptor in β1 suggests that one mechanism for the specificity differences β1 and β2 antagonists could be the presence of a H-bond donor group at the end of the heterocycle. This is indeed the case for nadolol and timolol, which have similar extended chain structures to both carazolol and cyanopindolol at their aminergic ends, but differ in their heterocyclic regions (FIG. 12).

Another significant effector of ligand specificity and the kinetics of ligand binding is EL2; the Cα positions within this highly structured region differ from β2 by an rmsd of 1 Å. There are also significant differences in the amino acid sequences between β1 and β2 in the entrance to the ligand binding region. This changes the shape of the entrance to the ligand binding region with a bridge formed by a H-bond between Asp192 and Lys305 in β2 that is absent in β1 because the respective residues are Glu200^{5.31} and Val312^{6.57}. Differences between β1 and β2 in this region could affect ligand selectivity in two ways. Firstly, some ligands have extensions that may make direct interactions with these sub-type specific residues. Secondly, the different physical characteristics of the entrance to the ligand binding region could affect the kinetics of ligand binding. Recent mutational studies not only show that EL2 defines the specificity, of allosteric modulators (Shi & Javitch 2004; Klco et al 2005; Scarselli et al 2007), but, in addition, the flexibility of the loop is important to the kinetics of modulator binding (Aviani et al 2007).

The structure of β1, when compared to β2, provides a sound basis for studying selectivity differences between RAR antagonists structurally similar to cyanopindolol and carazolol. However, many ligands, such as CGP 20712A and the agonist salmeterol, show very high selectivities (Baker 2005 BJP), but are structurally unrelated to either cyanopindolol or carazolol. These ligands could well bind to the βARs utilising additional amino acid residues to those described here. This is certainly the case for the binding of selective agonists such as for RO363 (Sugimoto at al, 2002) that cause a large conformational change upon binding; residues which are different between β1 and β2 and when mutated appear to be responsible for the differences in agonist affinity, are either distant from the cyanopindolol binding site on H2 facing the lipid phase (H β1AR L110^{(2.66)} and T117^{(2.63)}) or form a second shell cap (H β1AR F359^{(7.35)}) on the binding region (Sugimoto et al, 2002). Thus further structures with a variety of ligands bound will be required to fully understand all the complexities of ligand selectivity in the βARs.

### CONCLUSION

Two changes of consistently changed amino acids to more polar residues in beta 2 receptor close to the ligand site, and changes in the packing of amino acid side chains in the second shell of amino acid side chains which surrounds the antagonist ligand binding site modulate the detailed structure of the ligand binding site and must cause the observed differences in the pharmacological affinity profiles. These distant side chains are those which either make contact with the 14 side chains which do contact the ligand or are on the far side of the four transmembrane helices from which the 14 side chains protrude (H3, H5, H6, H7). Some of the more distant amino acid changes between β1AR and β2AR (also β3AR), of which there are over 100 highly subtype-conserved differences within the β-adrenergic family, must also contribute to the sub-type specificity. Thus the properties of the different members of the β-adrenergic GPCR subfamily in terms of pharmacology are due to the overall structure of the entire seven helix bundle with contributions from distant parts of the structure modulating the properties of the ligand binding site and its activation. Extrapolating to the related aminergic subfamilies and beyond, this implies that direct experimental observation of bound ligand structures will frequently be necessary and essential for successful design of selective drugs.

### Methods

### Purification and Crystallisation

The β1 receptor construct T34-424/His6 for baculovirus expression that was described in Warne et al (2003) was used as the basis for the generation of the β36/m23 construct used to determine the structure reported here. The construct was further truncated at the C-terminus after Leu367, and 6 Histidines were added to allow purification by Ni²⁺-affinity chromatography (IMAC). Two segments, comprising residues 244-271 and 277-278 of the third intracellular loop were also deleted. The construct included the following 8 point mutations: C116L increased expression, C358A removed palmitoylation and helped crystallisation, R68S, M90V, Y227A, A282L, F327A and F338M thermostabilise the receptor. Baculovirus expression in High 5™ cells, membrane preparation, solubilization, IMAC and alprenolol sepharose chromatography were all as previously described (Warne et al 2003), except that solubilization and IMAC were performed in buffers containing the detergent decylmaltoside and the detergent was exchanged on the alprenolol sepharose column to octylthioglucoside; purified receptor was eluted from the alprenolol sepharose with cyanopindolol (30 μM). The buffer was exchanged to 10 mM Tris-HCl pH7.7, 50 mM NaCl, 0.1 mM EDTA, 0.35% octylthioglucoside and 0.5 mM cyanopindolol during concentration to give a final receptor concentration of 5.5-6.0 mg/ml.

With the thermally stabilised protein first a wide crystalisation screen was performed in 4 different detergents. A total of 58 mg of receptor was used to set up 17800 crystallisation trials in MRC UV transparent crystallisation sitting drop plates that were and imaged with the MRC multi wavelength imaging system at 380 nm. Promising looking crystals were then observed at 280 nm to exclude salt and detergent crystals. 280 nm absorbing crystals were picked and X-rayed using a 4 um beam at ID 13 ESRF. The receptor crystallisation was then optimised manually by vapour diffusion at 18° C. with either hanging or sitting drop methodology after addition of an equal volume of reservoir solution (0.1M N-(2-acetamido)iminodiacetic acid (ADA), pH 6.9-7.3 and 29-32% PEG 600). Crystals were mounted on Hampton CrystalCap HT™ loops and frozen in liquid nitrogen. The best cryoprotection of crystals was achieved by increasing the PEG 600 concentration in the drop to 55-70%.

### Data Collection, Structure Solution and Refinement

The first diffraction patterns from microcrystals grown in the primary crystallisation screens were tested with a 5 μm beam at ID13 (Schertler & Riekel, 2005). The best crystallisation conditions were refined to improve diffraction quality and the optimised crystals were then screened at ID23-2 with a 10 μm focused beam; the micro-beams helped to deal with heterogeneous diffraction within a single crystal. Diffraction data were collected with a Mar 225 CCD detector on the microfocus beamline ID23-EH2 (λ=0.8726 Å) at the European Synchrotron Radiation Facility, Grenoble, using three positions on a single cryo-cooled crystal (100 K). The images were processed with MOSFLM (Leslie, Joint CCP4+ESF-EAMCB Newsletter on Protein Crystallography, No 26 (1992)) and SCALA (Acta Cryst D50: 760-763). The crystal initially diffracted to beyond 2.4 Å resolution, but radiation damage limited the final dataset resolution to 2.7 Å (Table 1).

The structure of turkey β₁AR-m23 was solved by molecular replacement with PHASER (McCoy et al (2007) J of App Cryst 40: 658-674), using the structure of human β₂AR (ref, PDB ID 2RH1) as an initial model. All four copies of the molecule in the triclinic unit cell were located. The amino acid sequence was corrected and the model was refined with PHENIX REFINE (Afonine et al (2005) CCP Newsletter, Contribution 8) and rebuilt with O (Jones et al (1991) Acta Cryst A47: 110-119). Tight non-crystallographic symmetry restraints (σ 0.025 Å) were applied to chains A and D and chains B and C. The cyanopindolol ligand, detergent and water molecules and the sodium ions were added at a late stage in the refinement. Final statistics are reported in Table 1.

TABLE 1

| Crystal ID | t1043 |
|---|---|
| Space group | P1 |
| Cell dimensions | |
| a, b, c (Å) | 55.5, 86.8, 95.5 |
| α, β, γ (°) | 67.6, 73.3, 85.8 |
| Data Processing | |
| Resolution (Å) | 45.1-2.7 |
| R_{merge} | 0.135 (0.666) |
| <I/σ (I)> | 5.8 (1.5) |
| Completeness (%) | 96.2 (95.7) |
| Multiplicity | 1.8 (1.8) |
| Wilson B (Å²) | 40.7 |
| Refinement | |
| Rwork | 0.226 |
| Rfree | 0.276 |
| r.m.s. deviation bonds (Å) | 0.011 |
| r.m.s. deviation angles (°) | 1.183 |

TABLE 2

Å

Molecule A

| | | | |
|---|---|---|---|
| Water 2 | Glu107 | OE2 | 3.56 |
| | Na+ | | 2.49 |
| Water 7 | Trp101 | O | 3.04 |
| | Leu105 | N | 3.41 |
| Water 8 | Arg140 | N | 3.45 |
| | Phe139 | N | 3.18 |
| Water 9 | Thr136 | O | 3.21 |
| Water 10 | Ser165 | O | 3.19 |
| | Val164 | O | 3.17 |
| | Tyr199 | OH | 2.54 |
| Water 11 | Glu107 | OE1 | 2.82 |
| | Trp174 | NE1 | 2.74 |
| | Ile169 | O | 2.76 |
| | Arg175 | NH2 | 3.19 |
| Water 12 | Arg197 | NH1 | 2.83 |
| | Phe298 | O | 3.54 |
| | B/Arg149 | NH2 | 3.54 |
| Water 13 | Cys285 | O | 2.86 |
| | Phe311 | O | 2.71 |
| | Phe289 | N | 2.7 |

Molecule B

| | | | |
|---|---|---|---|
| Water 12 | Arg149 | NH2 | 3.54 |
| | A/Arg197 | NH1 | 2.83 |
| | A/Phe298 | O | 3.54 |
| Water 14 | Trp99 | O | 2.7 |
| | Gly102 | N | 2.85 |
| | Pro188 | O | 2.76 |
| Water 15 | Cys191 | O | 3.21 |
| | Thr110 | OG1 | 2.77 |
| Water 16 | Arg197 | N | |
| Water 17 | Val303 | N | 2.83 |
| | Val303 | O | 3.39 |
| | Asn296 | OD1 | 2.39 |
| Water 18 | Asn318 | ND2 | 2.49 |
| | Trp286 | NE1 | 3.21 |

Molecule C

| | | | |
|---|---|---|---|
| Water 19 | Thr98 | O | 2.75 |
| | Leu100 | N | 3.02 |
| | Thr92 | OG1 | 2.54 |
| Water 20 | Trp99 | O | 2.8 |
| | Pro188 | O | 2.85 |
| | Gly102 | N | 2.83 |
| Water 21 | Thr110 | OG1 | 3.02 |
| Water 22 | Asp192 | OD1 | 3.12 |
| | Gly189 | O | 3.46 |
| | Cys191 | N | 2.64 |
| Water 23 | Trp286 | NE1 | 3.26 |
| | Asn318 | ND2 | 3.09 |

Molecule D

| | | | |
|---|---|---|---|
| Water 6 | Glu107 | OE2 | 3.5 |
| | Na+ ion | | 2.68 |
| Water 24 | Trp101 | O | 2.7 |
| | Leu105 | N | 3.04 |
| Water 25 | Ile169 | O | 2.76 |
| | Trp174 | NE1 | 2.65 |
| | Arg175 | NH2 | 3.26 |
| | Glu107 | OE1 | 2.88 |
| Water 26 | Gln186 | OE1 | 3.38 |
| Water 27 | Thr110 | OG1 | 3.21 |
| | Asp192 | O | 3.27 |
| Water 28 | Tyr199 | OH | 2.63 |
| | Ser165 | O | 2.92 |
| | Val164 | O | 3.35 |
| Water 29 | Phe311 | O | 2.84 |
| | Phe289 | N | 2.83 |
| | Cys285 | O | 2.65 |
| Water 30 | Gly315 | O | 2.43 |
| | Tyr316 | O | 3.37 |
| | Ser319 | OG | 2.99 |
| Water 31 | Asn322 | ND2 | 3.43 |
| | Tyr326 | OH | 2.96 |

“27 water molecules in total, 8 in A, 6 in B, 5 in C and 9 in D” (one shared between A & B; water 12)

TABLE 3

| Mutation | Stability (wild type = 100) |
|---|---|
| T144A | 72 |
| S145A | 68 |
| P146A | 13 |
| F147A | 128 |
| R148A | 89 |
| Y149A | 1 |
| Q150A | 117 |
| S151A | 117 |

### REFERENCES

Adams, P. D., R. W. Grosse-Kunstleve, L. W. Hung, T. R. Ioerger, A. J. McCoy, N. W. Moriarty, R. J. Read, J. C. Sacchettini, N. K. Sauter and T. C. Terwilliger (2002) PHENIX: building new software for automated crystallographic structure determination. Acta Crystallogr D. 58, 1948-1954.
Avlani, V. A., K. J. Gregory, C. J. Morton, M. W. Parker, P. M. Sexton and A. Christopoulos (2007) Critical role for the second extracellular loop in the binding of both orthosteric and allosteric G protein-coupled receptor ligands. J Biol. Chem. 282, 25677-86.
Bailey, S. (1994) The Ccp4 Suite—Programs for Protein Crystallography. Acta Crystallogr D. 50, 760-763.
Baker, J. G. (2005) The selectivity of beta-adrenoceptor antagonists at the human beta1, beta2 and beta3 adrenoceptors. Br J. Pharmacol. 144, 317-22.
Baldwin, J. M., G. F. Schertler and V. M. Unger (1997) An alpha-carbon template for the transmembrane helices in the rhodopsin family of G-protein-coupled receptors. J Mol. Biol. 272, 144-64.
Ballesteros, J. A., A. D. Jensen, G. Liapakis, S. G. Rasmussen, L. Shi, U. Gether and J. A. Javitch (2001) Activation of the beta β-adrenergic receptor involves disruption of an ionic lock between the cytoplasmic ends of transmembrane segments 3 and 6. J Biol. Chem. 276, 29171-7.
Ballesteros, J. A. and H. Weinstein (1995) Integrated methods for, the construction of three dimensional models and computational probing of structure function relations in G protein-coupled receptors. Methods in Neurosciences. Sealfon, S. C. and Conn, P. M. (eds.), pp 366-428, Academic Press San Diego, Calif.
Black, J. W. (1989) Drugs from Emasculated Hormones—the Principle of Syntopic Antagonism (Nobel Lecture). Angew Chem Int Edit. 28, 886-894.
Bockaert, J. and J. P. Pin (1999) Molecular tinkering of G protein-coupled receptors: an evolutionary success. EMBO J. 18, 1723-9.
Burstein, E. S., T. A. Spalding and M. R. Brann (1998) The second intracellular loop of the m5 muscarinic receptor is the switch which enables G-protein coupling. J Biol. Chem. 273, 24322-7.
Caron, M. G., Y. Srinivasan, J. Pitha, K. Kociolek and R. J. Lefkowitz (1979) Affinity chromatography of the beta-adrenergic receptor. J Biol. Chem. 254, 2923-7.
Cherezov, V., D. M. Rosenbaum, M. A. Hanson, S. G. Rasmussen, F. S. Thian, T. S. Kobilka, H. J. Choi, P. Kuhn, W. I. Weis, B. K. Kobilka and R. C. Stevens (2007) High-resolution crystal structure of an engineered human beta2-adrenergic G protein-coupled receptor. Science. 318, 1258-65.
Day, P. W., S. G. Rasmussen, C. Pamot, J. J. Fung, A. Masood, T. S. Kobilka, X. J. Yao, H. J. Choi, W. I. Weis, D. K. Rohrer and B. K. Kobilka (2007) A monoclonal antibody for G protein-coupled receptor crystallography. Nat. Methods. 4, 927-9.
Gether, U. (2000) Uncovering molecular mechanisms involved in activation of G protein-coupled receptors. Endocr Rev. 21, 90-113.
Gether, U., J. A. Ballesteros, R. Seifert, E. Sanders-Bush, H. Weinstein and B. K. Kobilka (1997) Structural instability of a constitutively active G protein-coupled receptor. Agonist-independent activation due to conformational flexibility. J Biol. Chem. 272, 2587-90.
Grisshammer, R., R. Duckworth and R. Henderson (1993) Expression of a rat neurotensin receptor in Escherichia coli. Biochem J. 295 (Pt 2), 571-6.
Harding, M. M. (2002) Metal-ligand geometry relevant to proteins and in proteins: sodium and potassium. Acta Crystallogr D Biol Crystallogr. 58, 872-4.
Isogaya, M., Y. Yamagiwa, S. Fujita, Y. Sugimoto, T. Nagao and H. Kurose (1998) Identification of a key amino acid of the beta-adrenergic receptor for high affinity binding of salmeterol. Mol. Pharmacol. 54, 616-22.
Jones, T. A., J. Y. Zou, S. W. Cowan and M. Kjeldgaard (1991) Improved Methods for Building Protein Models in Electron-Density Maps and the Location of Errors in These Models. Acta Crystallogr A. 47, 110-119.
Kikkawa, H., M. Isogaya, T. Nagao and H. Kurose (1998) The role of the seventh transmembrane region in high affinity binding of a beta(2)-selective agonist TA-2005. Mol Pharmacol. 53, 128-134.
Klco, J. M., C. B. Wiegand, K. Narzinski and T. J. Baranski (2005) Essential role for the second extracellular loop in C5a receptor activation., Nat Struct Mol. Biol. 12, 320-6.
Kobilka, B. and G. F. Schertler (2008) New G-protein-coupled receptor crystal structures: insights and limitations. Trends Pharmacol Sci.
Lattion, A., L. Abuin, M. Nenniger-Tosato and S. Cotecchia (1999) Constitutively active mutants of the beta1-adrenergic receptor. FEBS Lett. 4.57, 302-6.
Lewis, R. V. and C. Lofthouse (1993) Adverse Reactions with Beta-Adrenoceptor Blocking-Drugs—an Update. Drug Safety. 9, 272-279.
Li, J., P. C. Edwards, M. Burghammer, C. VIIIa and G. F. X. Schertler (2004) Structure of bovine rhodopsin in a trigonal crystal form. J Mol. Biol. 343, 1409-1438.
McCoy, A. J., R. W. Grosse-Kunstleve, P. D. Adams, M. D. Winn, L. C. Storoni and R. J. Read (2007) Phaser crystallographic software. Journal of Applied Crystallography. 40, 658-674.
Ostermeier, C. and H. Michel (1997) Crystallization of membrane proteins. Curr Opin Struct Biol. 7, 697-701.
Parker, E. M., K. Kameyama, T. Higashijima and E. M. Ross (1991) Reconstitutively active G protein-coupled receptors purified from baculovirus-infected insect cells. J Biol. Chem. 266, 519-27.
Perez, D. M. (2005) From plants to man: the GPCR “tree of life”. Mol. Pharmacol. 67, 1383-4.
Rasmussen, S. G., H. J. Choi, D. M. Rosenbaum, T. S. Kobilka, F. S. Thian, P. C. Edwards, M. Burghammer, V. R. Ratnala, R. Sanishvili, R. F. Fischetti, G. F. Schertler, W. I. Weis and B. K. Kobilka (2007) Crystal structure of the human beta2 adrenergic G-protein-coupled receptor. Nature. 450, 383-7.
Riekel, C., M. Burghammer and G. Schertler (2005) Protein crystallography microdiffraction. Curr Opin Struct Biol. 15, 556-62.
Rosenbaum, D. M., V. Cherezov, M. A. Hanson, S. G. Rasmussen, F. S. Thian, T. S. Kobilka, H. J. Choi, X. J. Yao, W. I. Weis, R. C. Stevens and B. K. Kobilka (2007) GPCR engineering yields high-resolution structural insights into beta2-adrenergic receptor function. Science. 318, 1266-73.
Scarselli, M., B. Li, S. K. Kim and J. Wess (2007) Multiple residues in the second extracellular loop are critical for M3 muscarinic acetylcholine receptor activation. J Biol. Chem. 282, 7385-96.
Serrano-Vega, M. J., F. Magnani, Y. Shibata and C. G. Tate (2008) Conformational thermostabilization of the beta1-adrenergic receptor in a detergent-resistant form. Proc Natl Acad Sci USA. 105, 877-82.
Shi, L. and J. A. Javitch (2004) The second extracellular loop of the dopamine D2 receptor lines the binding-site crevice. Proc Natl Acad Sci USA. 101, 440-5.
Shi, L., G. Liapakis, R. Xu, F. Guarnieri, J. A. Ballesteros and J. A. Javitch (2002) Beta2 adrenergic receptor activation. Modulation of the proline kink in transmembrane 6 by a rotamer toggle switch. J Biol. Chem. 277, 40989-96.
Sugimoto, Y., R. Fujisawa, R. Tanimura, A. L. Lattion, S. Cotecchia, G. Tsujimoto, T. Nagao and H. Kurose (2002) beta(1)-selective agonist (−)-1-(3,4-dimethoxyphenetylamino)-3-(3,4-dihydroxy)-2-propanol [(−)-RO363] differentially interacts with key amino acids responsible for beta(1)-selective binding in resting and active states. J Pharmacol Exp Ther. 301, 51-58.
Warne, T., J. Chirnside and G. F. Schertler (2003) Expression and purification of truncated, non-glycosylated turkey beta-adrenergic receptors for crystallization. Biochim Biophys Acta. 1610, 133-40.
Yarden, Y., H. Rodriguez, S. K. Wong, D. R. Brandt, D. C. May, J. Burnier, R. N. Harkins, E. Y. Chen, J. Ramachandran, A. Ullrich and et al. (1986) The avian beta-adrenergic receptor: primary structure and membrane topology. Proc Natl Acad Sci USA. 83, 6795-9.

### EXAMPLE 2

### Crystallisation of a Mutant Turkey β1-AR

### The Beta 36/m23 Crystallization Construct and Other Related Constructs

The Turkey beta-adrenergic receptor constructs Beta 34 and 36 are based on the previously described T34-424His6 construct [1], now renamed Beta 6. Beta 34 and 36, like Beta 6, are truncated at the N-terminus before residue 33, where the sequence MetGly has been added. Beta 34 & 36 are truncated at the C-terminus after Leu367, with the addition of a 6 histidine tag after the truncation. In Beta 36, two segments, comprising residues 244-271 and 277-278 of the third intracellular loop (ICL3) have also been deleted. All of the constructs incorporate the mutation C116L, which enhances expression [2]. Beta 34 and 36 both incorporate the mutation C358A, which eliminates the possibility of palmitoylation. The Beta 36/m23 crystallization construct includes in addition the six ‘m23’ mutations, R068S, M090V, Y227A, A282L, F327A and F338M, which enhance thermal/detergent stability [3]. Stabilized variants of Beta 6 (Beta 6/m23) and Beta 34 (Beta 34/m23) were also made by incorporating the six ‘m23’ mutations. A second version of Beta 36/m23 where C358 has not been mutated has also been made.

TABLE 4
Constructs.

| | | N-terminus | C-terminus | ICL 3 | m23 | |
|---|---|---|---|---|---|---|
| Construct | C116L | truncated | truncated | deleted | mutations | C358A |
| β6 | Yes | yes | no | no | no | no |
| β34 | Yes | yes | yes | no | no | yes |
| β36 | Yes | yes | yes | yes | no | yes |
| β6/m23 | Yes | yes | no | no | yes | no |
| β34/m23 | Yes | yes | yes | no | yes | yes |
| β36/m23 | Yes | yes | yes | yes | yes | yes |
| β36/m23/ | yes | yes | yes | yes | yes | no |
| C358 | | | | | | |

### Baculovirus Expression

The construct was expressed with the baculovirus system using Tni (High 5™) cells. The sequence CCCAAAATG was placed at the initiator methionine codon and the construct was subcloned into the baculovirus transfer vector pBacPAK8 (BD Clontech). The generation of recombinant baculovirus encoding Beta 36/m23 by co-transfection of Sf9 (S. frugiperda) cells, isolation of clonal virus, virus passage, and receptor expression in High 5™ cells were all as previously described [1].

### Beta 36 and Beta 36/m23 Purification, General Description

Insect cell membranes were prepared and solubilized as described previously [1], except that for the Beta 36/m23 construct, decylmaltoside (1.5%) was substituted for dodecylmaltoside as the solubilizing detergent after it had been established that subsequent detergent exchange was inefficient if dodecylmaltoside was used.

Purification was with first two column steps described for the T34-424His6 (Beta 6) construct [1], IMAC (Nickel) and alprenolol sepharose, which were run overnight at 5° C. It was found that the final size exclusion step which had been used for Beta 6 was not necessary for the Beta 36 constructs.

Beta 36 and Beta 36/m23 purification was performed on a small/medium or large scale, with the solubilization of insect cell membranes from 1L, 2L or 4L culture volume respectively. In either case a 10 ml, 1.6 cm diameter IMAC (Ni sepharose FF) column was used for the first step, as described previously for purification on a 2-5 mg scale [1]. For the small/medium scale, purification was continued with a 2.5 ml (1.6 cm diameter) aiprenolol sepharose column, for the large scale purification a 6 ml (2.6 cm diameter) column was used. Detergent exchange was performed on the alprenolol sepharose column, bound receptor was washed with buffer containing the new detergent. The previously utilized high salt (1M NaCl) wash was not used because octylthioglucoside (OTG), the detergent into which the receptor was exchanged for crystallization, is insoluble in high ionic strength buffers. As OTG also sometimes crystallized at 5° C., the aiprenolol sepharose wash buffer, which was used during the overnight FPLC procedure was maintained at 30° C. Other buffers containing OTG were only used for a short time or were of lower ionic strength than the aiprenolol sepharose wash buffer, and therefore problems with detergent solubility were not encountered. It was also found that it was not in fact necessary to warm the aiprenolol sepharose column in order to enhance the elution of beta-1 adrenergic receptor with the competing ligand, a measure which is recommended for beta-2 adrenergic receptor chromatography [4]. Eluted receptor fractions were concentrated with 100 kDa molecular weight cut-off (mwco) centricon concentrators (Millipore) to 1-2 ml. A buffer exchange step was then performed on a desalting column in to achieve the required (low) buffer and salt concentrations for crystallization experiments.

Cyanopindolol is quite expensive (£50/mg) and poorly soluble in aqueous buffers (0.75 mM). In order to increase the ligand concentration for crystallization, whilst minimizing costs, concentrated receptor was diluted with a buffer containing 0.69 mM cyanopindolol and then re-concentrated. The procedure was then repeated before final concentration of the receptor to at least 5 mg/ml with a cyanopindolol concentration of at least 0.5 mM. When using other less expensive ligands, such as (−) alprenolol, the dilution and re-concentration steps could be circumvented as it was possible to simply exchange the receptor into a buffer containing the required final ligand concentration on the desalting column and then concentrate it.

### Detailed Description of Chromatography and Subsequent Purification Steps, Purification for Crystallization in Octylthioglucoside

Buffer compositions are given in Table 5. Solubilized membrane proteins were applied to the 10 ml IMAC column at 0.35 ml/min. Total sample volumes were 60 ml, 120 ml or 180 ml for the purification of receptor from 1 L, 2L or 4L insect cells respectively. When sample loading was complete, the flow rate was increased to 1.85 ml/min and the column was washed with 80 ml IMAC A buffer. The imidazole concentration was increased to 27 mM (10% IMAC B buffer) with a linear gradient of 50 ml, and the column was further washed with 27 mM imidazole for 100 ml. The imidazole concentration was then rapidly increased to 250 mM (100% IMAC buffer) with a linear gradient of 20 ml, and elution was continued with 250 mM imidazole for a further 60 ml. Collection of a 65 ml volume which contained most of the receptor-1 binding activity was commenced as soon as the applied imidazole concentration had attained 150 mM. This partially-purified receptor fraction was then applied to a 2.5 ml, 1.6 cm diameter (1 or 2L scale purification) or 6 ml, 2.6 cm diameter (4L scale purification) alprenolol sepharose column.

### Alprenolol Sepharose Chromatography, Small/Medium Scale (1-2L Cells)

The 2.5 ml alprenolol sepharose column was loaded at a flow-rate of 0.25 ml/min. When sample loading was complete, the bound active fraction of the receptor was washed with 50 ml of Alprenolol sepharose wash buffer at 0.25 ml/min. The procedure was then paused for 1 hour before elution, giving the receptor a total of 4 hours exposure to the new detergent before elution. Elution was effected with 10 ml alprenolol sepharose elution buffer (+cyanopindolol) followed by a further 10 ml elution buffer (−cyanopindolol), all at a flow-rate of 0.4 ml/min. The eluted receptor was recovered in a 15 ml volume. UV monitoring of receptor elution was not possible due to the high absorbance of the ligand.

### Alprenolol Sepharose Chromatography, Large Scale (4L Cells)

The 6 ml, 2.6 cm diameter alprenolol sepharose column was loaded with partially purified receptor at 0.4 ml/min.

### Receptor Concentration, Buffer Exchange and Centrifugation Prior to Crystallization

Eluted receptor fractions were first concentrated 10-fold with 100 kDa mwco centricons to 1-1.5 ml. A sample was taken for protein estimation so that an estimate of the final yield and the required final volume could be made. Buffer was then exchanged to PD-10 buffer by application of the receptor to a pre-equilibrated G-25 sephadex PD-10 desalting column (GE Healthcare). The eluted receptor (2.5 ml) was then further concentrated with 100 kDa mwco centricons to ˜200 μl. The receptor was then diluted with 250 μl dilution buffer, reconcentrated to ˜200 μl, and the dilution repeated. The receptor was finally reconcentrated to 5-10 mg/ml, recovered from the centricons and then centrifuged at 60,000 rpm for 10 minutes at 4° C. to remove any possible aggregates. After final protein estimation, the receptor concentration was adjusted by addition of dilution buffer if necessary to achieve a final concentration of 5.0-6.5 mg/ml for crystallization.

TABLE 5
Buffers used in receptor purifications

| | Tris-HCl, | | Imidazole- | | | |
|---|---|---|---|---|---|---|
| Buffer | pH 7.7 | NaCl | HCl, pH 8 | EDTA | Detergent | Cyanopindolol³ |
| IMAC A | 20 mM | 350 mM | 2.5 mM | 0 | 0.15% DecM | 0 |
| IMAC B | 20 mM | 350 mM | 250 mM | 0 | 0.15% DecM | 0 |
| Alp. Sepharose | 20 mM | 350 mM | 0 | 1 mM | 0.4% OTG² | 0 |
| wash | | | | | | |
| Alp. sepharose | 20 mM | 350 mM | 0 | 0.2 mM | 0.35% OTG² | 30 μM |
| elution¹ | | | | | | |
| PD-10 exchange | 10 mM | 50 mM | 0 | 0.1 mM | 0.35% OTG² | 2 μM |
| buffer | | | | | | |
| Cyanopindolol | 10 mM | 50 mM | 0 | 0.1 mM | 0.35% OTG² | 0.69 mM |
| dilution buffer | | | | | | |
| Size exclusion | 20 mM | 50 mM | 0 | 0.5 mM | 0.35% OTG² | 2 μM |

DecM, decylmaltoside, OTG, octylthioglucoside
¹Alprenolol sepharose elution buffer was also prepared without cyanopindolol to continue elution of receptor, in order to minimize the quantity of ligand used
²Other detergents were also used for the later stages of purification, usually at a standard working concentration of 1.25 × cmc, eg fos-choline 10 (0.45%), hega 10 (0.35%) and nonylglucoside (0.28%)
³(-) alprenolol and other ligands were also used.

### Exchange to Other Detergents

A variety of other detergents could be used for Beta 36/m23 purification. A working concentration of 1.25×cmc was used throughout in all buffers.

### Size Exclusion Chromatography

Analytical size-exclusion chromatography was performed with on a Superdex 200 10/300 GL column. 100 μl samples were applied and run at 0.35 ml/min. The column was calibrated with the soluble protein standards ferritin (440 kDa), catalase (232 kDa), aldolase (158 kDa), BSA (67 kDa) and ovalbumin (43 kDa), which were run in the same buffer but without detergent. Preparative scale size-exclusion chromatography was performed with either a 16/60, for 1-4 mg receptor or with a 26/60 Superdex 200 column (4-10 mg receptor)

Size-exclusion chromatography was used as a final purification step in the preparation of Beta 6 and Beta 34 receptor constructs. When either of these constructs was eluted from a Superdex column, the main receptor peak, which was sharp and symmetrical, was preceded by smaller peaks comprising high molecular weight species which may have included aggregated receptor. When Beta 36 constructs were first purified, preparative size-exclusion chromatography was also used as a final purification step. However, a much improved elution profile was observed for Beta 36, along with an unusually late elution. Beta 36 also looked much cleaner on SDS PAGE when compared to both Beta 6 and Beta 34 constructs. For these reasons, size-exclusion chromatography was no longer considered to be a necessary step in the purification of Beta 36 constructs.

Analytical size-exclusion chromatography was routinely performed on Beta 36/m23 preparations as a quality control procedure and also to observe the effect on receptor properties after detergent exchange.

Apparent molecular weights of the Beta receptor constructs described were determined by size-exclusion chromatography on a calibrated column, as were the apparent molecular weights of Beta36/m23 in a variety of detergents. These results are listed in Table 6. Comparison of the apparent molecular weights of Beta 6, 34 & 36 in dodecylmaltoside with the predicted molecular weights of the respective constructs indicates that the behaviour of the Beta 36 construct has been dramatically altered, and it is possible that this is because the deletion of IC loop 3 has led to a reduced tendency to associate with itself and other proteins. When Beta 36/m23 was purified in the short-alkyl chain detergents which were used for crystallization, elution from the analytical size-exclusion column was later than when the receptor was eluted in dodecylmaltoside, indicating that the receptor was eluted in a detergent micelle which was significantly smaller (see FIG. 10). Because of the unusual behaviour of the Beta 36 construct, the apparent molecular weights of the receptor in these detergents was actually less than the calculated molecular weight of the construct.

TABLE 6
Size-exclusion data

| | | | mwt. app | Predicted |
|---|---|---|---|---|
| | Calculated | | with | mwt. of |
| | construct | | detergent | receptor in |
| Construct | mwt. (kDa) | Detergent | bound | micelle¹ |
| β6 | 45.06 | C12-maltoside | 120 | 122 |
| β34 | 39.25 | C12-maltoside | 103 | 116 |
| β36 | 35.95 | C12-maltoside | 79 | 112 |
| β36/m23/C358 | 35.74 | C10-maltoside | 53 | 69 |
| β36/m23 | 35.71 | C10-maltoside | 57.5 | 69 |
| β36/m23 | 35.71 | C9-maltoside | 47.5 | 61.5 |
| β36/m23 | 35.71 | C9-glucoside | 33.2 | n/a |
| β36/m23 | 35.71 | C8-S-glucoside | 28.1 | n/a |
| β36/m23 | 35.71 | LDAO | 64.3 | n/a |

¹The predicted weight of the receptor in the detergent micelle was calculated by addition of the molecular weight of the construct to the predicted mass of one detergent micelle; aggregation numbers for the respective detergents determined by the detergent manufacturer, Anatrace, were used to predict the following micellar masses: dodecylmaltoside, 77.6 kDa; decylmaltoside, 33.3 kDa; nonylmaltoside, 25.7 kDa.

### Crystallization of Beta 36/m23

Crystallization was by the vapour diffusion method at 18° C. Receptor was diluted 1:1 with precipitant solution and crystallized on either MRC 96-well plates with the sitting drop method (200 nl or 500 nl receptor) or Qiagen easy xtal dg (dropguard) plates for hanging drops (1 μl receptor).

Beta 36/m23 purified in 0.35% OTG with 0.5 mM cyanopindolol crystallized over a wide pH range (5.6-9.5) and with a large variety of PEGs at concentrations of 25-35% as precipitant with the addition of wide range of salts. The best diffracting crystals with receptor purified in OTG were obtained with 0.1M ADA (N-(2-acetaimido) iminodiacetic acid) buffer, pH6.9-7.3 and 29-32% PEG 600 as precipitant. Crystals usually appeared within 24-48 hours, and crystal growth was complete within 72 hours. Initial crystal screening for crystallization conditions and the first rounds of optimization were with MRC sitting drop plates. However, crystals grown under hanging drop conditions on the Qiagen plates showed improved morphology and were easier to mount in cryoloops for freezing. Dropguard coverslips were used, the smaller of the two well sizes was appropriate for the 1 μl+1 μl drops. The use of the dropguard well restricted drop spreading and suppressed nucleation, possibly by restricting the surface area of the drop and slowing vapour diffusion. Larger crystals could be grown in this way than could be grown with either MRC sitting drop plates, sitting drops on microbridges, or conventional coverslips for hanging drops.

Diffracting crystals of Beta 36/m23 could also be grown with receptor purified in nonylglucoside, fos-choline 10 and hega 10, but crystallization conditions for these detergents have not so far been optimized. However, in all three cases the best conditions are in the pH range 7-8.5 with ˜30% PEG as precipitant.

### Crystal Freezing and Cryoprotection

Crystals were mounted on Hampton CrystalCap HT™ loops and frozen with liquid nitrogen. It was presumed that the PEG 600 concentration in the crystallization drop was insufficient to give good cryoprotection, so the PEG concentration in the drop was increased to 70% in initial freezing attempts. As a variable unit cell size was observed, a cryoprotectant solution comprising either 40% PEG 600 or 35% PEG 600 and 5% glycerol was used in order to reduce variation of the unit cell due to dehydration of the crystal. Finally it was observed that it was not necessary to add any cryoprotectant to the drop, and many crystals were successfully frozen this way in order to preserve isomorphism. However, high resolution better than 3 Å was never seen in these crystals, therefore PEG concentrations of 50-70% were used for crystal freezing.

### REFERENCES

[1] Warne, T, Chimside, J., and Schertler, G. F. (2003) Expression and purification of truncated, non-glycosylated turkey beta-adrenergic receptors for crystallization, Biochim. Biophys. Acta. 1610, 133-40.
[2] Parker, E. M., Kameyama, K., Higashijima, T. and Ross, E. M. (1991) J. Biol. Chem. 266 (1), 519-27.
[3] Serrano-Vega, M. J., Magnani, F., Shibata, Y., Tate, C. G. (2008) Proc Natl Accd Sci USA. 105 (3), 877-82
[4] Caron, M. G., Srinivasan, Y., Pitha, J., Kociolek, K. and Lefkowitz, R. J. (1979) J. Biol. Chem. 254 (8), 2923-27.

### EXAMPLE 3

### RMSD Calculations

### A. Rmsd Calculation Between β2-AR Structures

RMSD Between PDB code: 2RH1 and PDB Code: 2R4S After LSQMAN Alignment (the 2R4S Structure is of Poor Quality and Low Resolution)
(using only residues for alignment in H2-H6 as follows)
Helix 2 69-90 (residue numbering from beta2)

### Helix 3 109-134

### Helix 4 148-164

### Helix 5 200-229

### Helix 6 269-291

### Helix 7 311-323

Overall rmsd=0.74 Å on 384 main chain atoms, used in alignment (this large deviation is due almost entirely to inaccuracies in 2R4S)

Overall rmsd=1.38 Å on 552 main chain atoms, but many loops and uncertain regions were omitted in the 2R4S publication

Helix 1 1.01 Å on 63 atoms
Helix 2 0.81 Å on 45 atoms

Helix 4 0.58 Å on 51 atoms

Helix 5 0.76 Å on 57 atoms
Helix 6 0.43 Å on 66 atoms
Helix 7 0.89 Å on 48 atoms
Cytoplasmic loop-1 0.60 Å on 18 atoms
Extracellular loop-1 1.09 Å on 42 atoms
Cytoplasmic loop-2 1.25 Å on 30 atoms
Extracellular loop-2 0.98 Å on 15 atoms
Cytoplasmic loop-3 4.37 Å on 30 atoms
Extracellular loop—no residues remain in the 2R4S in this region; none have been built
Helix 8 3.10 Å on12 atoms

### B. Rmsd Calculation Between β1-AR (Molecule B) and β2-AR

RMSD Between Beta1 molB and 2RH1 After LSQMAN Alignment
(using residues only in H2-H6 for alignment as follows)
Helix 2 69-90 (residue numbering from beta2)

### Helix 3 109-134

### Helix 4 148-164

### Helix 5 200-229

### Helix 6 269-291

### Helix 7 311-323

Overall rmsd=0.399 Å on 426 main chain atoms (Cα, C, N) used in alignment in H2-H6

Overall rmsd=1.235 Å on 801 main chain atoms (Cα, C, N) in complete structure

Helix 1 0.606 Å on 63 atoms
Helix 2 0.416 Å on 6 atoms
Helix 3 0.304 Å on 78 atoms
Helix 4 0.550 Å on 54 atoms
Helix 5 0.401 Å on 90 atoms
Helix 6 0.403 Å on 75 atoms
Helix 7 0.310 Å on 63 atoms
Cytoplasmic loop-1 0.796 Å on 27 atoms
Extra cellular loop-1 0.732 Å on 54 atoms
Cytoplasmic loop-2 4.830 Å on 39 atoms
Extracellular loop-2 0.836 Å on 102 atoms
Cytoplasmic loop-3 0.721 Å on 9 atoms
Extracellular loop-3 0.985 Å on 27 atoms
Helix 8 1.018 Å on 54 atoms

### C. Rmsd Calculation Between β1-AR Molecules A and B

RMSD Between Beta1 molB and Beta1 molA After LSQMAN Alignment
(alignment used only residues in H2-H6 as follows)
Helix 2 69-90 (residue numbering from beta2)

### Helix 3 109-134

### Helix 4 148-164

### Helix 5 200-229

### Helix 6 269-291

### Helix 7 311-323

Overall rmsd=0.314 Å on 426 main chain atoms in H2-H6 (Cα, C, N) used in alignment

Overall rmsd=0.465 Å on 792 main chain atoms from complete structure, excluding N-terminal part of H1.

Helix 1 2.185 Å on 63 atoms (all of H1—large because of the 60° kink of N-terminus before residue 42)
Helix 2 0.312 Å on 6 atoms
Helix 3 0.230 Å on 78 atoms
Helix 4 0.388 Å on 54 atoms
Helix 5 0.341 Å on 90 atoms
Helix 6 0.230 Å on 75 atoms
Helix 7 0.378 Å on 63 atoms
Cytoplasmic loop-1 0.599 Å on 27 atoms
Extracellular loop-1 0.418 Å on 54 atoms
Cytoplasmic loop-2 0.468 Å on 39 atoms
Extracellular loop-2 0.633 Å on 102 atoms
Cytoplasmic loop-3 0.261 Å on 9 atoms (most of this very large loop deleted from coordinates)
Extracellular loop-3 0.694 Å on 27 atoms
Helix 8 0.510 Å on 54 atoms

### D. RMSD Calculation Between β1-AR (Molecule B) and β2-AR (2RN1)

### Comparison of the Active Site Residues Between β1 and β2

| | B2 residue | B1 residue | B-W |
|---|---|---|---|
| AA residue | number | number | number |
| Trp | 109 | 117 | 3.28 |
| Thr | 110 | 118 | 3.29 |
| Asp | 113 | 121 | 3.32 |
| Val | 114 | 122 | 3.33 |
| Val | 117 | 125 | 3.36 |
| Phe | 193 | 201 | 5.32 |
| Thr | 195 | 203 | 5.34 |
| Tyr | 199 | 207 | 5.38 |
| Ser | 203 | 211 | 5.42 |
| Ser | 207 | 215 | 5.46 |
| Phe | 289 | 306 | 6.51 |
| Phe | 290 | 307 | 6.52 |
| Asn | 293 | 310 | 6.55 |
| Asn | 312 | 329 | 7.39 |

The β1 and β2 receptors were aligned based upon helices 2-7. The RMS difference between the position of the 14 ligand binding residues in β1 and β2 were then determined. For comparison, the RMS difference between the same residue in an alignment of β1 molecule A and β1 molecule B (molB) was performed.

Considering only Cα atoms, the RMSD between β1 molB and β2 is 0.4 Å compared to 0.2 Å when the two β1 molecules are compared.

Considering only side chain atoms, the RMSD between β1 molb and β2 is 0.6 Å compared to 0.3 Å when the two β1 molecules are compared.

### Methods

The above rmsd calculations were performed using the following LSQMAN script:—

#!/bin/csh -f
#
# note that residue numbering here refers to human beta2
# sequence and homologous residues in beta1
#
lsqman <<eof
re BETA1 /ss1/rh15/MolB_bar_8feb08-lig-Na—H2O.pdb
re BETA2 /ss1/rh15/2RH1_BAR_res.pdb
li
at ma
ex BETA1 “A69-A90 A109-A134 A148-A165 A200-A229 A269-A293
A303-A323” BETA2 “A69 A109 A148 A200 A269 A303”
at ca
rmsd BETA1 “A109-A110 A113-A114 A117 A193 A195 A199 A203
A207 A289-290 A293 A312” BETA2 “A109 A113 A117 A193 A195
A199 A203 A207 A289 A293 A312”
at ma
rmsd BETA1 “A109-A110 A113-A114 A117 A193 A195 A199 A203
A207 A289-290 A293 A312” BETA2 “A109 A113 A117 A193 A195
A199 A203 A207 A289 A293 A312”
at all
rmsd BETA1 “A109-A110 A113-A114 A117 A193 A195 A199 A203
A207 A289-290 A293 A312” BETA2 “A109 A113 A117 A193 A195
A199 A203 A207 A289 A293 A312”
at side
rmsd BETA1 “A109-A110 A113-A114 A117 A193 A195 A199 A203
A207 A289-290 A293 A312” BETA2 “A109 A113 A117 A193 A195
A199 A203 A207 A289 A293 A312”
quit
eof
#]

Alignments and comparisons were obtained using LSQMAN:
G. J. Kleywegt & T. A. Jones (1994). A super position.

### CCP4/ESF-EACBM Newsletter on Protein Crystallography 31,

November 1994, pp. 9-14. [http://xray.bmc.uu.se/usf/factory_{—}4.html]

### EXAMPLE 4

Turkey β1-AR is a member of the GPCR superfamily and its homology to many other known and potential drug targets can be used to build 3D models of such targets, which may also contain known ligands docked into the protein structure, by a process of homology modelling (Blundell et al (Eur. J. Biochem, Vol. 172, (1988), 513). These models can then be used in turn to select for binding partners, in particular small-molecule drug-like compounds, which are predicted to bind to the target in question. Such compounds are then either synthesised or, if they already exist and are available, tested for activity in biochemical or functional, assays. If they show the desired potency they may then be progressed for further screening, for example in in vivo pharmacology assays, or alternatively subjected to further rounds of chemistry or biosynthetic modification prior to testing in a succession of assays. In this fashion the turkey β1-AR structure can be used to enable the discovery of novel drug candidates.

Protein modelling is a well established technique that begins with an alignment of the target protein or its relevant orthologue (in this case GPCR with preferably but not necessarily >30% sequence identity across the transmembrane helical regions, for example human beta-1 adrenergic receptor, human beta-2 adrenergic receptor, human beta-3 adrenergic receptor, human dopamine D2 receptor, human muscarinic M1-M5 receptors, other aminergic receptors, human or rat neurotensin receptor, human adenosine Ata receptor) with β1-AR using an algorithm such as BLAST, preferably in the University of Washington implementation WU-BLAST (WU-BLAST version 2.0 executable programs for several UNIX platforms can be downloaded from ftp://blast. wustl. edu/blast/executables). This program is based on WU-BLAST version 1.4, which in turn is based on the public domain NCBI-BLAST version 1.4 (Altschul and Gish, 1996, Local alignment statistics, Doolittle ed., Methods in Enzymology 266: 460-480; Altschul et al., 1990, Basic local alignment search tool, Journal of Molecular Biology 215: 403-410; Gish and States, 1993, Identification of protein coding regions by database similarity search, Nature Genetics 3: 266-272; Karlin and Altschul, 1993, Applications and statistics for multiple high-scoring segments in molecular sequences, Proc. Natl. Acad. Sci. USA 90: 5873-5877.

In all search programs in the suite the gapped alignment routines are integral to the database search itself. Gapping can be turned off if desired. The default penalty (O) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Once the amino acid sequences of turkey β1-AR and the target protein of unknown structure have been aligned, the structures of the conserved amino acids in the structural representation of the turkey β1-AR may be transferred to the corresponding amino acids of the target protein. For example, a tyrosine in the amino acid sequence of turkey β1-AR may be replaced by a phenylalanine, the corresponding homologous amino acid in the amino acid sequence of the target protein.

The structures of amino acids located in non-conserved regions may be assigned manually by using standard peptide geometries or by molecular simulation techniques, such as molecular dynamics (Lee, M. R.; Duan, Y.; Kollman, P. A. State of the art in studying protein folding and protein structure prediction using molecular dynamics methods. Journal of Molecular Graphics & Modelling (2001), 19(1), 146-149). The final step in the process is accomplished by refining the entire structure using molecular dynamics and/or energy minimization. Typically, the predicted three dimensional structural representation will be one in which favourable interactions are formed within the target protein and/or so that a low energy conformation is formed.

Typically, homology modelling is performed using computer programs, for example SWISS MODEL available through the Swiss Institute for Bioinformatics in Geneva, Switzerland; WHATIF available on EMBL servers; Schnare et al. (1996) J. Mol. Biol, 256: 701-719; Blundell et al. (1987) Nature 326: 347-352; Fetrow and Bryant (1993) Bio/Technology 11:479-484; Greer (1991) Methods in Enzymology 202: 239-252; and Johnson et al (1994) Crit. Rev. Biochem. Mol. Biol. 29:1-68. An example of homology modelling is described in Szklarz G. D (1997) Life Sci. 61: 2507-2520.

Binding partners such as known agonists or antagonists, or molecules that may be agonists or antagonists, or simply molecules that it is thought may have the potential to interact with the receptor target can then be docked into the protein model, typically by positioning of a 3D representation of the candidate binding partner in the anticipated ligand binding region, by analogy with the cyanopindolol binding region delineated in the cyanopindolol/beta-1AR co-structure presented herein (Table A, B, C or D). Known or putative binding partners may then be modified stepwise, alternatively binding partners may be designed de novo using the empty or partly occupied binding site, or these two approaches may be combined.

In order to provide a three-dimensional structural representation of a candidate binding partner, the binding partner structural representation may be modelled in three dimensions using commercially available software for this purpose or, if its crystal structure is available, the coordinates of the structure may be used to provide a structural representation of the binding partner.

The design of binding partners that bind to a β1-AR or a model based on β1-AR generally involves consideration of two factors.

First, the binding partner must be capable of physically and structurally associating with parts or all of a β1-AR potential or known binding region or homologous parts of a modeled target receptor. Non-covalent molecular interactions important in this association include hydrogen bonding, van der Waals interactions, hydrophobic interactions and electrostatic interactions.

Second, the binding partner must be able to assume a conformation that allows it to associate with a binding region directly. Although certain portions of the binding partner will not directly participate in these associations, those portions of the binding partner may still influence the overall conformation of the molecule. This, in turn, may have a significant impact on potency. Such conformational requirements include the overall three-dimensional structure and orientation of the binding partner in relation to all or a portion of the binding region, or the spacing between functional groups of a binding partner comprising several binding partners that directly interact with the β1-AR or homologous target.

Thus it will be appreciated that selected coordinates which represent a binding region of the turkey β1-AR, e.g. atoms from amino acid residues contributing to the ligand binding site including amino acid residues 117, 118, 121, 122, 125, 201, 203, 207, 211, 215, 306, 307, 310 and 329 may be used. Additional preferences for the selected coordinates are as defined above with respect to the first aspect of the invention.

Designing of binding partners can generally be achieved in two ways, either by the step wise assembly of a binding partner or by the de novo synthesis of a binding partner.

With respect to the step-wise assembly of a binding partner, several methods may be used. Typically the process begins by visual inspection of, for example, any of the binding regions on a computer representation of the turkey β1-AR as defined by the coordinates in Table. A, Table B, Table C or Table D optionally varied within a rmsd of residue backbone atoms of not more than 1.235 Å, or selected coordinates thereof. Selected binding partners, or fragments or moieties thereof may then be positioned in a variety of orientations, or docked, within the binding region. Docking may be accomplished using software such as QUANTA and Sybyl (Tripos Associates, St. Louis, Mo.), followed by, or performed simultaneously with, energy minimization, rigid-body minimization (Gshwend, supra) and molecular dynamics with standard molecular mechanics force fields, such as CHARMM and AMBER.

Specialized computer programs may also assist in the process of selecting binding partners or fragments or moieties thereof. These include: 1. GRID (P. J. Goodford, “A Computational Procedure for Determining Energetically Favorable Binding Sites on Biologically Important Macromolecules”, J. Med. Chem., 28, pp. 849-857 (1985)). GRID is available from Oxford University, Oxford, UK. 2. MCSS (A. Miranker et al., “Functionality Maps of Binding Sites: A Multiple Copy Simultaneous Search Method.” Proteins: Structure, Function and Genetics, 11, pp. 29-34 (1991)). MCSS is available from Molecular Simulations, San Diego, Calif. 3. AUTODOCK (D. S. Goodsell et al., “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif. 4. DOCK (I. D. Kuntz et al., “A Geometric Approach to Macromolecule-Ligand Interactions”, J. Mol. Biol., 161, pp. 269-288 (1982)). DOCK is available from University of California, San Francisco, Calif.

Once suitable binding partners or fragments have been selected, they may be assembled into a single compound or complex. Assembly may be preceded by visual inspection of the relationship of the fragments to each other on the three-dimensional image displayed on a computer screen in relation to the structure coordinates of the turkey β1-AR or a model of an homologous target. This would be followed by manual model building using software such as QUANTA or Sybyl.

Useful programs to aid one of skill in the art in connecting the individual chemical entities or fragments include: 1. CAVEAT (P. A. Bartlett et al., “CAVEAT: A Program to Facilitate the Structure-Derived Design of Biologically Active Molecules”, in “Molecular Recognition in Chemical and Biological Problems”, Special Pub., Royal Chem. Soc., 78, pp. 182-196 (1989); G. Lauri and P. A. Bartlett, “CAVEAT: a Program to Facilitate the Design of Organic Molecules”, J. Comput. Aided Mol. Des., 8, pp. 51-66 (1994)). CAVEAT is available from the University of California, Berkeley, Calif.; 2. 3D Database systems such as ISIS (MDL Information Systems, San Leandro, Calif.). This area is reviewed in Y. C. Martin, “3D Database Searching in Drug Design”, J. Med. Chem., 35, pp. 2145-2154 (1992); and 3. HOOK (M. B. Eisen et al., “HOOK: A Program for Finding Novel Molecular Architectures that Satisfy the Chemical and Steric Requirements of a Macromolecule Binding Site”, Proteins: Struct., Funct., Genet., 19, pp. 199-221 (1994). HOOK is available from Molecular Simulations, San Diego, Calif.

Thus the invention includes a method of designing a binding partner of a β1-AR or an homologous target model comprising the steps of: (a) providing a structural representation of a β1-AR binding region as defined by the coordinates of turkey β1-AR of Table A, Table B, Table C or Table D, optionally varied by a root mean square deviation of residue backbone atoms of not more than 1.235 Å or selected coordinates thereof (b) using computational means to dock a three dimensional structural representation of a first binding partner in part of the binding region; (c) docking at least a second binding partner in another part of the binding region; (d) quantifying the interaction energy between the first or second binding partner and part of the binding region; (e) repeating steps (b) to (d) with another first and second binding partner, selecting a first and a second binding partner based on the quantified interaction energy of all of said first and second binding partners; (f) optionally, visually inspecting the relationship of the first and second binding partner to each other in relation to the binding region; and (g) assembling the first and second binding partners into a one binding partner that interacts with the binding region by model building.

As an alternative to the step-wise assembly of binding partners, binding partners may be designed as a whole or “de novo” using either an empty binding region or optionally including some portion(s) of a known binding partner(s). There are many de novo ligand design methods including: 1. LUDI (H.-J. Bohm, “The Computer Program LUDI: A New Method for the De Novo Design of Enzyme Inhibitors”, J. Comp. Aid. Molec. Design, 6, pp. 61-78 (1992)). LUDI is available from Molecular Simulations Incorporated, San Diego, Calif.; 2. LEGEND (Y. Nishibata et al., Tetrahedron, 47, p. 8985 (1991)). LEGEND is available from Molecular Simulations Incorporated, San Diego, Calif.; 3. LeapFrog (available from Tripos Associates, St. Louis, Mo.); and 4. SPROUT (V. Gillet et al., “SPROUT: A Program for Structure Generation)”, J. Comput. Aided Mol. Design, 7, pp. 127-153 (1993)). SPROUT is available from the University of Leeds, UK.

Other molecular modelling techniques may also be employed in accordance with this invention (see, e.g., N. C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)).

In addition to the methods described above in relation to the design of binding partners, other computer-based methods are available to select for binding partners that interact with β1-AR.

For example the invention involves the computational screening of small molecule databases for binding partners that can bind in whole, or in part, to the turkey β1-AR or an homologous target model. In this screening, the quality of fit of such binding partners to a binding region of a β1-AR site as defined by the coordinates of turkey β1-AR of Table A, Table B, Table C or Table D, optionally varied by a root mean square deviation of residue backbone atoms of not more than 1.235 Å or selected coordinates thereof, may be judged either by shape complementarity or by estimated interaction energy (E. C. Meng et al., J. Comp. Chem., 13, pp. 505-524 (1992)).

For example, selection may involve using a computer for selecting an orientation of a binding partner with a favourable shape complementarity in a binding region comprising the steps of: (a) providing the coordinates of turkey β1-AR of Table A, Table B, Table C or Table D, optionally varied by a root mean square deviation of residue backbone atoms of not more than 1.235 Å or selected coordinates thereof and a three-dimensional structural representation of one or more candidate binding partners; (b) employing computational means to dock a first binding partner in the binding region; (c) quantitating the contact score of the binding partner in different orientions; and (d) selecting an orientation with the highest contact score.

The docking may be facilitated by the contact score. The method may further comprise the step of generating a three-dimensional structural repsentation of the binding region and binding partner bound therein prior to step (b).

The method may further, comprise the steps of: (e) repeating steps (b) through (d) with a second binding partner; and (f) selecting at least one of the first or second binding partner that has a higher contact score based on the quantitated contact score of the first or second binding partner.

In another embodiment, selection may involve using a computer for selecting an orientation of a binding partner that interacts favourably with a binding region comprising; a) providing the coordinates of turkey β1-AR of Table A, Table B, Table C or Table D, optionally varied by a root mean square deviation of residue backbone atoms of not more than 1.235 Å or selected coordinates thereof; b) employing computational means to dock a first binding partner in the binding region; c) quantitating the interaction energy between the binding partner and all or part of a binding region for different orientations of the binding partner; and d) selecting the orientation of the binding partner with the most favorable interaction energy.

The docking may be facilitated by the quantitated interaction energy and energy minimization with or without molecular dynamics simulations may be performed simultaneously with or following step (b).

The method may further comprise the steps of: (e) repeating steps (b) through (d) with a second binding partner; and (f) selecting at least one of the first or second binding partner that interacts more favourably with a binding region based on the quantitated interaction energy of the first or second binding partner.

In another embodiment, selection may involve screening a binding partner to associate at a deformation energy of binding of less than −7 kcal/mol with a β1-AR binding region comprising: (a) providing the coordinates of turkey β1-AR of Table A, Table B, Table C or Table D, optionally varied by a root mean square deviation of residue backbone atoms of not more than 1.235 Å or selected coordinates thereof and employing computational means which utilise coordinates to dock the binding partner into a binding region; (b) quantifying the deformation energy of binding between the binding partner and the binding region; and (d) selecting a binding partner that associates with a β1-AR binding region at a deformation energy of binding of less than −7 kcal/mol.

The potential binding effect of a binding partner on β1-AR may be analysed prior to its actual synthesis and testing by the use of computer modeling techniques. If the theoretical structure of the given entity suggests insufficient interaction and association between it and the β1-AR, testing of the entity is obviated. However, if computer modelling indicates a strong interaction, the molecule may then be synthesized and tested for its ability to bind to a β1-AR. In this manner, synthesis of inoperative compounds may be avoided.

The compound is then tested in a physical drug screen such as a radioligand binding assay, a fluorescent ligand binding assay, a whole cell functional assay for example by measuring cAMP upregulation, or a large range of other possible assays well known to those skilled in the art. The choice of assay is highly dependent on the target GPCR.

Once drug-like hit or lead molecules have been identified they may be modified by iterative medicinal chemistry. Co-crystallisation or soaking of crystals of turkey beta-1 AR with these “leads” would be a useful guide to their binding modes, and such information is fed into molecular modeling and design as described at the start of this Example (Example 4).

Binding surfaces for macromolecules, for example G-proteins or antibodies, might also be predicted using the structure of beta-1 AR or of homology models based on it.

### Tables A-D

Tables A-D show the x, y and z coordinates by amino acid residue of each non-hydrogen atom in the polypeptide structure for molecules A, B, C and D respectively, in addition to the antagonist cyanopindolol atoms. The fourth column indicates whether the atom is from an amino acid residue of the protein (by 3-letter amino acid code eg TRP, GLU, ALA etc), the cyanopindolol ligand (PDL), a sodium atom (NA), a water molecule (HOH), octyithioglucoside molecule (8TG)¹ or a decylmaltoside atom (DMU)¹ (¹Molecule D only).

Parameters used in the modelling of the turkey β1-AR are provided below:

REMARK Date 2008-02-08 Time 12:58:22 GMT +0000
REMARK PHENIX refinement
REMARK ****************** SUMMARY OF INPUT REFLECTION DATA ******************
REMARK Reflections:

| | |
|---|---|
| REMARK file name | :bar_t1043_a_trunc_201to1040_27A_unique1.mtz |
| REMARK labels | :[‘F_bar_t1043, SIGF_bar_t1043, DANO, SIGDANO’] |
| REMARK resolution d_max | :45.1367 A |
| REMARK resolution d_min | :2.7001 A |

| | |
|---|---|
| REMARK number of reflections total | :72699 |
| REMARK number of reflections work | :69129 (percent from total = 95.09) |
| REMARK number of reflections test | :3570 (percent from total = 4.91) |

| | |
|---|---|
| REMARK completeness | :0.8391 (in range: 45.1367-2.7001 A) |
| REMARK R-free flags: | |

| | |
|---|---|
| REMARK file name | :bar_t1043_a_trunc_201to1040_27A_unique1.mtz |
| REMARK label | :FreeR_flag |

REMARK test_flag_value: 1
REMARK Experimental phase information: Not available
REMARK *********************** SUMMARY OF INPUT MODEL ***********************
REMARK Model file name(s):
REMARK /andrewgrp0/andrew/gpcr/bar/esrf_7dec07/rebuild_8feb.pdb

| | |
|---|---|
| REMARK Number of atoms | :8913 |
| REMARK Unit cell volume | :407066.316 |
| REMARK Space group | :1 (P 1) |

REMARK Number of symmetries : 1
REMARK |-ADP statistics (Wilson B = 38.602)------------------------------|
REMARK |Atom |Number of | isotropic or equivalent| Anisotropy |min/max |
REMARK |type |iso aniso |min max mean | min max mean |
REMARK |------|---------|--------------|------------|
REMARK |Solv+Mac: 8913 0 10.55 170.05 46.37 None None None |
REMARK |Sol. :27 0 40.00 40.00 40.00 None None None |
REMARK |Mac. :8886 0 10.55 170.05 46.39 None None None |
REMARK |Hyd. :0 0 None None None None None None |
REMARK |------------------------------------------- |
REMARK | Distribution of isotropic (or equivalent) ADP for non-H atoms:|
REMARK |Bin# value range #atoms | Bin# value range #atoms |
REMARK | 0: 10.550-26.500: 1339 | 5: 90.300-106.250: 320 |
REMARK | 1: 26.500-42.450: 3750 | 6: 106.250-122.200: 158 |
REMARK | 2: 42.450-58.400: 1834 | 7: 122.200-138.150: 91 |
REMARK | 3: 58.400-74.350: 857 | 8: 138.150-154.100: 27 |
REMARK | 4: 74.350-90.300: 533 | 9: 154.100-170.050: 4 |
REMARK | =>continue=> |
REMARK |-------------------------------------------------------------|
REMARK |-Geometry statistics-----------------------------------|
REMARK |Type | Deviation from ideal | Targets |Target (sum) |
REMARK | | mean max min| | |
REMARK |bond | 0.025 0.820 0.000| 9858.434| |
REMARK |angle | 2.000 70.460 0.000| 8178.315| |
REMARK |chirality| 0.090 0.413 0.000| 295.520| 28592.299 |
REMARK |planarity| 0.019 0.340 0.000| 2731.746| |
REMARK |dihedral|25.199 174.976 0.001| 5701.449 | |
REMARK |nonbonded| 4.533 5.540 1.491| 1826.836 | |
REMARK |------------------------------------------------|
REMARK |------------------------------------------------|
REMARK | Histogram of deviations from ideal values for |
REMARK |Bonds |Angles |Nonbonded contacts|

| REMARK \|0.000-0.082: | 9080\| 0.000-7.046: 12340\|1.491-1.896: 3\| | |
|---|---|---|
| REMARK \|0.082-0.164: | 9\| 7.046-14.092: | 59\|1.896-2.301: 12\| |
| REMARK \|0.164-0.246: | 7\| 14.092-21.138: | 4\|2.301-2.706: 456\| |
| REMARK \|0.246-0.328: | 7\| 21.138-28.184: | 0\|2.706-3.110: 7093\| |
| REMARK \|0.328-0.410: | 5\| 28.184-35.230: | 0\|3.110-3.515: 8679\| |
| REMARK \|0.410-0.492: | 9\| 35.230-42.276: | 0\|3.515-3.920: 14282\| |
| REMARK \|0.492-0.574: | 0\| 42.276-49.322: | 0\|3.920-4.325: 15158\| |
| REMARK \|0.574-0.656: | 0\| 49.322-56.368: | 0\|4.325-4.730: 21695\| |
| REMARK \|0.656-0.738: | 0\| 56.368-63.414: | 0\|4.730-5.135: 24834\| |
| REMARK \|0.738-0.820: | 2\| 63.414-70.460: | 4\|5.135-5.540: 27926\| |

REMARK |--------------------------------------|
REMARK ******************** REFINEMENT SUMMARY: QUICK FACTS ********************
REMARK Start: r_work = 0.2422 r_free = 0.2792 bonds = 0.025 angles = 2.000
REMARK Final: r_work = 0.2264 r_free = 0.2759 bonds = 0.011 angles = 1.183
REMARK **********************************************************************

| | |
|---|---|
| REMARK Refinement target | : ml |
| REMARK Calculation algorithm | :fft |

| | |
|---|---|
| REMARK Use sin/cos table | :False |

REMARK Statistics in bins for work reflections:
REMARK Bin Resolution Compl. No. Scale_k1(work) R-factor(work)
REMARK number range refl.

| | | | | | |
|---|---|---|---|---|---|
| REMARK 1: | 45.1430-13.8198 | 0.77 | 474 | 0.507 | 0.3670 |
| REMARK 2: | 13.8198-11.0217 | 0.86 | 541 | 0.431 | 0.2138 |
| REMARK 3: | 11.0217-9.6440 | 0.83 | 518 | 0.419 | 0.1873 |
| REMARK 4: | 9.6440-8.7693 | 0.83 | 533 | 0.423 | 0.1731 |
| REMARK 5: | 8.7693-8.1447 | 0.85 | 551 | 0.419 | 0.1710 |
| REMARK 6: | 8.1447-7.6669 | 0.79 | 480 | 0.414 | 0.1828 |
| REMARK 7: | 7.6669-7.2846 | 0.81 | 513 | 0.402 | 0.2505 |
| REMARK 8: | 7.2846-6.9687 | 0.86 | 523 | 0.390 | 0.2300 |
| REMARK 9: | 6.9687-6.7013 | 0.84 | 528 | 0.384 | 0.2409 |
| REMARK 10: | 6.7013-6.4708 | 0.81 | 537 | 0.390 | 0.2345 |
| REMARK 11: | 6.4708-6.2690 | 0.83 | 483 | 0.397 | 0.2555 |
| REMARK 12: | 6.2690-6.0902 | 0.80 | 519 | 0.390 | 0.2314 |
| REMARK 13: | 6.0902-5.9302 | 0.84 | 520 | 0.386 | 0.2375 |
| REMARK 14: | 5.9302-5.7859 | 0.83 | 555 | 0.391 | 0.2266 |
| REMARK 15: | 5.7859-5.6546 | 0.82 | 483 | 0.388 | 0.2331 |
| REMARK 16: | 5.6546-5.5345 | 0.83 | 502 | 0.396 | 0.2144 |
| REMARK 17: | 5.5345-5.4239 | 0.78 | 517 | 0.397 | 0.2102 |
| REMARK 18: | 5.4239-5.3217 | 0.81 | 511 | 0.406 | 0.2178 |
| REMARK 19: | 5.3217-5.2268 | 0.82 | 507 | 0.421 | 0.1957 |
| REMARK 20: | 5.2268-5.1384 | 0.81 | 532 | 0.414 | 0.1925 |
| REMARK 21: | 5.1384-5.0556 | 0.79 | 433 | 0.425 | 0.1947 |
| REMARK 22: | 5.0556-4.9779 | 0.79 | 525 | 0.428 | 0.1900 |
| REMARK 23: | 4.9779-4.9048 | 0.81 | 531 | 0.442 | 0.1859 |
| REMARK 24: | 4.9048-4.8358 | 0.83 | 497 | 0.434 | 0.1702 |
| REMARK 25: | 4.8358-4.7705 | 0.80 | 483 | 0.445 | 0.1860 |
| REMARK 26: | 4.7705-4.7086 | 0.83 | 564 | 0.452 | 0.1787 |
| REMARK 27: | 4.7086-4.6498 | 0.82 | 481 | 0.461 | 0.1718 |
| REMARK 28: | 4.6498-4.5938 | 0.82 | 489 | 0.467 | 0.1841 |
| REMARK 29: | 4.5938-4.5405 | 0.83 | 544 | 0.460 | 0.1638 |
| REMARK 30: | 4.5405-4.4895 | 0.81 | 551 | 0.467 | 0.1795 |
| REMARK 31: | 4.4895-4.4408 | 0.81 | 485 | 0.479 | 0.1827 |
| REMARK 32: | 4.4408-4.3941 | 0.80 | 501 | 0.473 | 0.1807 |
| REMARK 33: | 4.3941-4.3493 | 0.84 | 512 | 0.477 | 0.1683 |
| REMARK 34: | 4.3493-4.3062 | 0.82 | 497 | 0.482 | 0.2027 |
| REMARK 35: | 4.3062-4.2649 | 0.81 | 516 | 0.473 | 0.1830 |
| REMARK 36: | 4.2649-4.2250 | 0.81 | 504 | 0.477 | 0.1708 |
| REMARK 37: | 4.2250-4.1866 | 0.78 | 513 | 0.474 | 0.1919 |
| REMARK 38: | 4.1866-4.1496 | 0.83 | 487 | 0.492 | 0.1905 |
| REMARK 39: | 4.1496-4.1139 | 0.82 | 533 | 0.487 | 0.1658 |
| REMARK 40: | 4.1139-4.0793 | 0.79 | 526 | 0.479 | 0.1790 |
| REMARK 41: | 4.0793-4.0459 | 0.80 | 468 | 0.461 | 0.2039 |
| REMARK 42: | 4.0459-4.0136 | 0.82 | 537 | 0.476 | 0.1710 |
| REMARK 43: | 4.0136-3.9822 | 0.85 | 479 | 0.487 | 0.1784 |
| REMARK 44: | 3.9822-3.9519 | 0.85 | 527 | 0.482 | 0.1788 |
| REMARK 45: | 3.9519-3.9224 | 0.77 | 525 | 0.475 | 0.1845 |
| REMARK 46: | 3.9224-3.8938 | 0.80 | 527 | 0.476 | 0.1793 |
| REMARK 47: | 3.8938-3.8660 | 0.78 | 491 | 0.474 | 0.1935 |
| REMARK 48: | 3.8660-3.8390 | 0.81 | 489 | 0.464 | 0.1744 |
| REMARK 49: | 3.8390-3.8127 | 0.83 | 518 | 0.474 | 0.1729 |
| REMARK 50: | 3.8127-3.7871 | 0.78 | 488 | 0.473 | 0.1796 |
| REMARK 51: | 3.7871-3.7622 | 0.80 | 492 | 0.473 | 0.1835 |
| REMARK 52: | 3.7622-3.7379 | 0.85 | 553 | 0.453 | 0.1781 |
| REMARK 53: | 3.7379-3.7143 | 0.83 | 499 | 0.465 | 0.1802 |
| REMARK 54: | 3.7143-3.6912 | 0.81 | 486 | 0.463 | 0.1792 |
| REMARK 55: | 3.6912-3.6687 | 0.79 | 505 | 0.467 | 0.1779 |
| REMARK 56: | 3.6687-3.6468 | 0.80 | 531 | 0.460 | 0.1919 |
| REMARK 57: | 3.6468-3.6253 | 0.79 | 468 | 0.464 | 0.1954 |
| REMARK 58: | 3.6253-3.6044 | 0.83 | 569 | 0.460 | 0.1950 |
| REMARK 59: | 3.6044-3.5839 | 0.83 | 524 | 0.450 | 0.2064 |
| REMARK 60: | 3.5839-3.5639 | 0.81 | 485 | 0.456 | 0.1803 |
| REMARK 61: | 3.5639-3.5443 | 0.80 | 479 | 0.454 | 0.2114 |
| REMARK 62: | 3.5443-3.5252 | 0.81 | 534 | 0.436 | 0.2086 |
| REMARK 63: | 3.5252-3.5064 | 0.82 | 485 | 0.447 | 0.2267 |
| REMARK 64: | 3.5064-3.4881 | 0.80 | 533 | 0.445 | 0.2026 |
| REMARK 65: | 3.4881-3.4701 | 0.80 | 446 | 0.445 | 0.2176 |
| REMARK 66: | 3.4701-3.4525 | 0.79 | 507 | 0.443 | 0.2202 |
| REMARK 67: | 3.4525-3.4353 | 0.79 | 519 | 0.438 | 0.2234 |
| REMARK 68: | 3.4353-3.4183 | 0.77 | 486 | 0.442 | 0.2201 |
| REMARK 69: | 3.4183-3.4018 | 0.78 | 494 | 0.439 | 0.2056 |
| REMARK 70: | 3.4018-3.3855 | 0.80 | 513 | 0.432 | 0.2028 |
| REMARK 71: | 3.3855-3.3695 | 0.83 | 554 | 0.420 | 0.2303 |
| REMARK 72: | 3.3695-3.3539 | 0.80 | 506 | 0.435 | 0.2238 |
| REMARK 73: | 3.3539-3.3385 | 0.83 | 478 | 0.436 | 0.2589 |
| REMARK 74: | 3.3385-3.3234 | 0.79 | 469 | 0.436 | 0.2351 |
| REMARK 75: | 3.3234-3.3086 | 0.81 | 505 | 0.436 | 0.2252 |
| REMARK 76: | 3.3086-3.2940 | 0.78 | 492 | 0.431 | 0.2556 |
| REMARK 77: | 3.2940-3.2797 | 0.79 | 494 | 0.436 | 0.2206 |
| REMARK 78: | 3.2797-3.2656 | 0.82 | 524 | 0.437 | 0.2250 |
| REMARK 79: | 3.2656-3.2518 | 0.78 | 502 | 0.439 | 0.2313 |
| REMARK 80: | 3.2518-3.2382 | 0.83 | 554 | 0.428 | 0.2364 |
| REMARK 81: | 3.2382-3.2248 | 0.80 | 461 | 0.417 | 0.2308 |
| REMARK 82: | 3.2248-3.2116 | 0.78 | 486 | 0.423 | 0.2364 |
| REMARK 83: | 3.2116-3.1987 | 0.77 | 486 | 0.428 | 0.2450 |
| REMARK 84: | 3.1987-3.1859 | 0.79 | 509 | 0.436 | 0.2354 |
| REMARK 85: | 3.1859-3.1734 | 0.76 | 498 | 0.423 | 0.2426 |
| REMARK 86: | 3.1734-3.1611 | 0.80 | 494 | 0.424 | 0.2308 |
| REMARK 87: | 3.1611-3.1489 | 0.81 | 553 | 0.416 | 0.2461 |
| REMARK 88: | 3.1489-3.1369 | 0.75 | 449 | 0.424 | 0.2294 |
| REMARK 89: | 3.1369-3.1251 | 0.82 | 505 | 0.417 | 0.2370 |
| REMARK 90: | 3.1251-3.1135 | 0.77 | 452 | 0.411 | 0.2345 |
| REMARK 91: | 3.1135-3.1021 | 0.78 | 449 | 0.414 | 0.2380 |
| REMARK 92: | 3.1021-3.0908 | 0.79 | 466 | 0.414 | 0.2603 |
| REMARK 93: | 3.0908-3.0797 | 0.78 | 515 | 0.406 | 0.2378 |
| REMARK 94: | 3.0797-3.0687 | 0.79 | 529 | 0.407 | 0.2376 |
| REMARK 95: | 3.0687-3.0579 | 0.78 | 512 | 0.423 | 0.2520 |
| REMARK 96: | 3.0579-3.0473 | 0.80 | 526 | 0.407 | 0.2527 |
| REMARK 97: | 3.0473-3.0368 | 0.81 | 496 | 0.398 | 0.2465 |
| REMARK 98: | 3.0368-3.0264 | 0.80 | 493 | 0.400 | 0.2503 |
| REMARK 99: | 3.0264-3.0162 | 0.80 | 486 | 0.403 | 0.2406 |
| REMARK 100: | 3.0162-3.0061 | 0.79 | 509 | 0.405 | 0.2662 |
| REMARK 101: | 3.0061-2.9962 | 0.80 | 472 | 0.410 | 0.2533 |
| REMARK 102: | 2.9962-2.9863 | 0.79 | 507 | 0.413 | 0.2550 |
| REMARK 103: | 2.9863-2.9767 | 0.82 | 509 | 0.415 | 0.2555 |
| REMARK 104: | 2.9767-2.9671 | 0.82 | 525 | 0.403 | 0.2507 |
| REMARK 105: | 2.9671-2.9576 | 0.76 | 518 | 0.389 | 0.2509 |
| REMARK 106: | 2.9576-2.9483 | 0.81 | 501 | 0.391 | 0.2681 |
| REMARK 107: | 2.9483-2.9391 | 0.78 | 452 | 0.403 | 0.2776 |
| REMARK 108: | 2.9391-2.9300 | 0.79 | 461 | 0.400 | 0.2547 |
| REMARK 109: | 2.9300-2.9210 | 0.78 | 473 | 0.411 | 0.2655 |
| REMARK 110: | 2.9210-2.9121 | 0.76 | 512 | 0.406 | 0.2775 |
| REMARK 111: | 2.9121-2.9034 | 0.78 | 462 | 0.402 | 0.2802 |
| REMARK 112: | 2.9034-2.8947 | 0.80 | 522 | 0.400 | 0.2794 |
| REMARK 113: | 2.8947-2.8861 | 0.79 | 538 | 0.398 | 0.2835 |
| REMARK 114: | 2.8861-2.8777 | 0.78 | 481 | 0.397 | 0.2668 |
| REMARK 115: | 2.8777-2.8693 | 0.80 | 501 | 0.399 | 0.2632 |
| REMARK 116: | 2.8693-2.8611 | 0.77 | 481 | 0.384 | 0.2565 |
| REMARK 117: | 2.8611-2.8529 | 0.79 | 532 | 0.404 | 0.2903 |
| REMARK 118: | 2.8529-2.8448 | 0.79 | 476 | 0.403 | 0.2808 |
| REMARK 119: | 2.8448-2.8368 | 0.75 | 492 | 0.392 | 0.2642 |
| REMARK 120: | 2.8368-2.8289 | 0.80 | 491 | 0.380 | 0.2620 |
| REMARK 121: | 2.8289-2.8211 | 0.80 | 469 | 0.395 | 0.2657 |
| REMARK 122: | 2.8211-2.8134 | 0.80 | 467 | 0.395 | 0.2819 |
| REMARK 123: | 2.8134-2.8057 | 0.79 | 492 | 0.414 | 0.2939 |
| REMARK 124: | 2.8057-2.7982 | 0.80 | 499 | 0.401 | 0.2750 |
| REMARK 125: | 2.7982-2.7907 | 0.81 | 551 | 0.392 | 0.3078 |
| REMARK 126: | 2.7907-2.7833 | 0.77 | 499 | 0.387 | 0.3175 |
| REMARK 127: | 2.7833-2.7760 | 0.77 | 452 | 0.390 | 0.3239 |
| REMARK 128: | 2.7760-2.7687 | 0.77 | 449 | 0.397 | 0.3138 |
| REMARK 129: | 2.7687-2.7615 | 0.75 | 509 | 0.399 | 0.3012 |
| REMARK 130: | 2.7615-2.7544 | 0.77 | 487 | 0.389 | 0.3305 |
| REMARK 131: | 2.7544-2.7474 | 0.77 | 477 | 0.392 | 0.3205 |
| REMARK 132: | 2.7474-2.7405 | 0.74 | 474 | 0.406 | 0.3250 |
| REMARK 133: | 2.7405-2.7336 | 0.76 | 431 | 0.396 | 0.3534 |
| REMARK 134: | 2.7336-2.7268 | 0.73 | 515 | 0.394 | 0.3721 |
| REMARK 135: | 2.7268-2.7200 | 0.73 | 439 | 0.393 | 0.3536 |
| REMARK 136: | 2.7200-2.7133 | 0.76 | 480 | 0.402 | 0.3558 |
| REMARK 137: | 2.7133-2.7067 | 0.72 | 455 | 0.411 | 0.3558 |
| REMARK 138: | 2.7067-2.7002 | 0.73 | 470 | 0.443 | 0.3594 |

REMARK where:
REMARK R-factor = SUM(||Fobs|−Scale_k1 * |Fmodel||)/SUM(|Fobs|)
REMARK Scale_k1 = SUM(|Fobs| * |Fmodel|)/SUM(|Fmodel|**2)
REMARK Fmodel = fb_cart * (Fcalc + Fbulk)
REMARK Fbulk = k_sol * exp(−b_sol * s**2/4) * Fmask
REMARK Fcalc = structure factors calculated from atomic model
REMARK fb_cart = exp(−h(t) * A(−1) * B_cart * A(−1t) * h),
REMARK A - orthogonalization matrix
REMARK |−ADP statistics (Wilson B = 38.602)--------------------|
REMARK | Atom | Number of | Isotropic or equivalent| Anisotropy |min/max |
REMARK | type |iso aniso | min max mean | min max mean |
REMARK | ----|-------|-------------|-------------|
REMARK | Solv+Mac: 8913 0 11.29 162.50 46.52 None None None |
REMARK | Sol. :27 0 13.55 60.94 33.46 None None None |
REMARK | Mac. :8886 0 11.29 162.50 46.56 None None None |
REMARK | Hyd. :0 0 None None None None None None |
REMARK | ------------------------------------------------ |
REMARK | Distribution of isotropic (or equivalent) ADP for non-H atoms: |
REMARK | Bin# value range #atoms | Bin# value range #atoms |
REMARK | 0: 11.293-26.413: 1281 | 5: 86.894-102.015: 347 |
REMARK | 1: 26.413-41.533: 3688 | 6: 102.015-117.135: 200 |
REMARK | 2: 41.533-56.654: 1823 | 7: 117.135-132.255: 108 |
REMARK | 3: 56.654-71.774: 837 | 8: 132.255-147.375: 62 |
REMARK | 4: 71.774-86.894: 554 | 9: 147.375-162.496: 13 |
REMARK | =>continue=> |
REMARK |-------------------------------------------------------------|
REMARK |-Geometry statistics-----------------------------------|
REMARK |Type | Deviation from ideal | Targets |Target (sum) |
REMARK | | mean max min | | |
REMARK |bond | 0.011 0.380 0.000| 914.190| |
REMARK |angle | 1.183 11.356 0.000| 3117.897| |
REMARK |chirality| 0.075 0.374 0.000 | 204.632| 10760.461 |
REMARK |planarity| 0.005 0.048 0.000| 190.107| |
REMARK |dihedral | 25.170 170.893 0.007| 5289.495| |
REMARK |nonbonded| 4.315 5.475 2.231| 1044.139| |
REMARK |------------------------------------------------|
REMARK |------------------------------------------------|
REMARK | Histogram of deviations from ideal values for |
REMARK |Bonds |Angles |Nonbonded contacts|
REMARK |0.000-0.038: 9102| 0.000-1.136: 9825|2.231-2.555: 43|
REMARK |0.038-0.076: 5| 1.136-2.271: 1815|2.555-2.880: 3490|
REMARK |0.076-0.114: 2| 2.271-3.407: 477|2.880-3.204: 5853|
REMARK |0.114-0.152: 0| 3.407-4.543: 179|3.204-3.529: 7001|
REMARK |0.152-0.190: 4| 4.543-5.678: 69|3.529-3.853: 11830|
REMARK |0.190-0.228: 3| 5.678-6.814: 25|3.853-4.177: 10222|
REMARK |0.228-0.266: 1|6.814-7.949: 7|4.177-4.502: 16163|
REMARK |0.266-0.304: 0|7.949-9.085: 7|4.502-4.826: 18060|
REMARK |0.304-0.342: 1|9.085-10.221: 1|4.826-5.151: 20389|
REMARK |0.342-0.380: 1|10.221-11.356: 2|5.151-5.475: 4494|
REMARK |------------------------------------------------|
REMARK ****************** REFINEMENT STATISTICS STEP BY STEP ******************
REMARK leading digit, like 1_, means number of macro-cycle
REMARK 0 :statistics at the very beginning when nothing is done yet
REMARK 1_bss: bulk solvent correction and/or (anisotropic) scaling
REMARK 1_xyz: refinement of coordinates
REMARK 1_adp: refinement of ADPs (Atomic Displacement Parameters)
REMARK 1_sar: simulated annealing refinement of x, y, z
REMARK -------------------------------------------------------------
REMARK R-factors, x-ray target values and norm of gradient of x-ray target
REMARK stage r-work r-free xray_target_w xray_target_t
REMARK 0 :0.3647 0.3686 4.744063e+00 4.810621e+00
REMARK 1_bss: 0.2422 0.2792 4.652633e+00 4.733425e+00
REMARK 1_xyz: 0.2264 0.2812 4.617832e+00 4.733139e+00
REMARK 1_adp: 0.2226 0.2783 4.601622e+00 4.723398e+00
REMARK 2_bss: 0.2233 0.2754 4.601172e+00 4.717059e+00
REMARK 2_xyz: 0.2287 0.2762 4.615511e+00 4.716959e+00
REMARK 2_sar: 0.2292 0.2757 4.617258e+00 4.717022e+00
REMARK 2_xyz: 0.2277 0.2765 4.613917e+00 4.717557e+00
REMARK 2_adp: 0.2261 0.2767 4.609129e+00 4.717554e+00
REMARK 3_bss: 0.2258 0.2762 4.608872e+00 4.717243e+00
REMARK 3_xyz: 0.2266 0.2761 4.610807e+00 4.716857e+00
REMARK 3_adp: 0.2268 0.2764 4.610185e+00 4.716700e+00
REMARK 3_bss: 0.2264 0.2759 4.610043e+00 4.716476e+00
REMARK -------------------------------------------------------------
REMARK Weights for target T = Exray * wxc * wxc_scale + Echem * wc and
REMARK angles between gradient vectors, eg. (d_Exray/d_sites, d_Echem/d_sites)
REMARK stage wxc wxu wxc_sc wxu_sc /_gxc, gc /_gxu, gu
REMARK 0 : 1.1624e+01 1.9406e−01 0.500 1.000 92.954 108.526
REMARK 1_bss: 1.1624e+01 1.9406e−01 0.500 1.000 92.954 108.526
REMARK 1_xyz: 1.1498e+01 1.7959e−01 0.500 1.000 92.865 109.494
REMARK 1_adp: 1.1498e+01 1.7959e−01 0.500 1.000 92.865 109.494
REMARK 2_bss: 1.1498e+01 1.7959e−01 0.500 1.000 92.865 109.494
REMARK 2_xyz: 3.6207e+00 1.8788e−01 0.500 1.000 149.180 154.067
REMARK 2_sar: 3.6207e+00 1.8788e−01 0.500 1.000 149.180 154.067
REMARK 2_xyz: 3.6207e+00 1.8788e−01 0.500 1.000 149.180 154.067
REMARK 2_adp: 3.6207e+00 1.8788e−01 0.500 1.000 149.180 154.067
REMARK 3_bss: 3.6207e+00 1.8788e−01 0.500 1.000 149.180 154.067
REMARK 3_xyz: 3.1559e+00 1.8905e−01 0.500 1.000 165.525 158.557
REMARK 3_adp: 3.1559e+00 1.8905e−01 0.500 1.000 165.525 158.557
REMARK 3_bss: 3.1559e+00 1.8905e−01 0.500 1.000 165.525 158.557
REMARK -------------------------------------------------------------
REMARK stage k_sol b_sol b11 b22 b33 b12 b13 b23
REMARK 0 : 0.000 0.000 0.000 0.000 0.000 0.000 0.000 0.000
REMARK 1_bss: 0.336 44.351 −4.572 9.380 −4.076 0.798 0.420 −2.128
REMARK 1_xyz: 0.336 44.351 −4.572 9.380 −4.076 0.798 0.420 −2.128
REMARK 1_adp: 0.336 44.351 −4.572 9.380 −4.076 0.798 0.420 −2.128
REMARK 2_bss: 0.337 44.351 −3.446 10.002 −2.498 0.680 0.454 −2.138
REMARK 2_xyz: 0.337 44.351 −3.446 10.002 −2.498 0.680 0.454 −2.138
REMARK 2_sar: 0.337 44.351 −3.446 10.002 −2.498 0.680 0.454 −2.138
REMARK 2_xyz: 0.337 44.351 −3.446 10.002 −2.498 0.680 0.454 −2.138
REMARK 2_adp: 0.337 44.351 −3.446 10.002 −2.498 0.680 0.454 −2.138
REMARK 3_bss: 0.337 44.484 −2.739 10.984 −1.520 0.618 0.478 −2.262
REMARK 3_xyz: 0.337 44.484 −2.739 10.984 −1.520 0.618 0.478 −2.262
REMARK 3_adp: 0.337 44.484 −2.739 10.984 −1.520 0.618 0.478 −2.262
REMARK 3_bss: 0.338 47.639 −5.103 8.843 −3.740 0.599 0.483 −2.334
REMARK -------------------------------------------------------------
REMARK stage <pher> fom alpha beta
REMARK 0 : 32.034 0.7531 0.3593 3972.999
REMARK 1_bss: 28.896 0.7883 0.3815 2730.148
REMARK 1_xyz: 29.216 0.7842 0.3789 2776.956
REMARK 1_adp: 28.686 0.7901 0.3715 2711.543
REMARK 2_bss: 28.395 0.7932 0.3811 2630.430
REMARK 2_xyz: 28.204 0.7957 0.3830 2624.650
REMARK 2_sar: 28.178 0.7960 0.3831 2632.925
REMARK 2_xyz: 28.195 0.7958 0.3831 2633.352
REMARK 2_adp: 28.130 0.7966 0.3761 2630.693
REMARK 3_bss: 28.127 0.7966 0.3826 2628.991
REMARK 3_xyz: 28.065 0.7974 0.3830 2627.379
REMARK 3_adp: 28.043 0.7976 0.3764 2627.304
REMARK 3_bss: 28.046 0.7976 0.3831 2626.244
REMARK -------------------------------------------------------------
REMARK stage angl bond chir dihe plan repu geom_target wc
REMARK 0 : 2.000 0.025 0.090 25.199 0.019 4.533 2.8592e+04 1.00
REMARK 1_bss: 2.000 0.025 0.090 25.199 0.019 4.533 2.8592e+04 1.00
REMARK 1_xyz: 2.184 0.024 0.124 25.897 0.009 4.315 2.7383e+04 1.00
REMARK 1_adp: 2.184 0.024 0.124 25.897 0.009 4.315 2.7383e+04 1.00
REMARK 2_bss: 2.184 0.024 0.124 25.897 0.009 4.315 2.7383e+04 1.00
REMARK 2_xyz: 1.285 0.012 0.079 25.338 0.005 4.317 1.2130e+04 1.00
REMARK 2_sar: 1.478 0.014 0.088 25.417 0.006 4.315 1.5055e+04 1.00
REMARK 2_xyz: 1.287 0.012 0.080 25.206 0.005 4.315 1.2018e+04 1.00
REMARK 2_adp: 1.287 0.012 0.080 25.206 0.005 4.315 1.2018e+04 1.00
REMARK 3_bss: 1.287 0.012 0.080 25.206 0.005 4.315 1.2018e+04 1.00
REMARK 3_xyz: 1.183 0.011 0.075 25.170 0.005 4.315 1.0760e+04 1.00
REMARK 3_adp: 1.183 0.011 0.075 25.170 0.005 4.315 1.0760e+04 1.00
REMARK 3_bss: 1.183 0.011 0.075 25.170 0.005 4.315 1.0760e+04 1.00
REMARK -------------------------------------------------------------
REMARK Maximal deviations:
REMARK stage angl bond chir dihe plan repu |grad|
REMARK 0 :70.460 0.820 0.413 174.976 0.340 1.491 2.4900e−01
REMARK 1_bss: 70.460 0.820 0.413 174.976 0.340 1.491 2.4900e−01
REMARK 1_xyz: 18.309 0.587 0.511 173.972 0.079 2.063 8.4482e−02
REMARK 1_adp: 18.309 0.587 0.511 173.972 0.079 2.063 8.4482e−02
REMARK 2_bss: 18.309 0.587 0.511 173.972 0.079 2.063 8.4482e−02
REMARK 2_xyz: 11.636 0.470 0.373 173.016 0.048 2.240 3.1598e−02
REMARK 2_sar: 11.173 0.375 0.450 174.183 0.045 2.143 6.2998e−02
REMARK 2_xyz: 11.532 0.445 0.387 169.382 0.048 2.206 3.1147e−02
REMARK 2_adp: 11.532 0.445 0.387 169.382 0.048 2.206 3.1147e−02
REMARK 3_bss: 11.532 0.445 0.387 169.382 0.048 2.206 3.1147e−02
REMARK 3_xyz: 11.356 0.380 0.374 170.893 0.048 2.231 3.1049e−02
REMARK 3_adp: 11.356 0.380 0.374 170.893 0.048 2.231 3.1049e−02
REMARK 3_bss: 11.356 0.380 0.374 170.893 0.048 2.231 3.1049e−02
REMARK -------------------------------------------------------------
REMARK |-----overall-----|---macromolecule----|------solvent------|
REMARK stage b_max b_min b_ave b_max b_min b_ave b_max b_min b_ave
REMARK 0 :170.05 10.55 46.37 170.05 10.55 46.39 40.00 40.00 40.00
REMARK 1_bss: 170.05 10.55 46.37 170.05 10.55 46.39 40.00 40.00 40.00
REMARK 1_xyz: 170.05 10.55 46.37 170.05 10.55 46.39 40.00 40.00 40.00
REMARK 1_adp: 157.99 9.40 44.98 157.99 9.40 45.01 57.42 16.23 33.87
REMARK 2_bss: 157.99 9.40 44.98 157.99 9.40 45.01 57.42 16.23 33.87
REMARK 2_xyz: 157.99 9.40 44.98 157.99 9.40 45.01 57.42 16.23 33.87
REMARK 2_sar: 157.99 9.40 44.98 157.99 9.40 45.01 57.42 16.23 33.87
REMARK 2_xyz: 157.99 9.40 44.98 157.99 9.40 45.01 57.42 16.23 33.87
REMARK 2_adp: 159.29 8.52 44.14 159.29 8.52 44.18 58.11 11.66 31.36
REMARK 3_bss: 159.29 8.52 44.14 159.29 8.52 44.18 58.11 11.66 31.36
REMARK 3_xyz: 159.29 8.52 44.14 159.29 8.52 44.18 58.11 11.66 31.36
REMARK 3_adp: 159.34 8.14 43.37 159.34 8.14 43.41 57.78 10.40 30.30
REMARK 3_bss: 162.50 11.29 46.52 162.50 11.29 46.56 60.94 13.55 33.46
REMARK -------------------------------------------------------------
REMARK stage Deviation of refined
REMARK model from start model
REMARK max min mean
REMARK 0 : 0.000 0.000 0.000
REMARK 1_bss: 0.000 0.000 0.000
REMARK 1_xyz: 2.097 0.006 0.133
REMARK 1_adp: 2.097 0.006 0.133
REMARK 2_bss: 2.097 0.006 0.133
REMARK 2_xyz: 2.036 0.004 0.132
REMARK 2_sar: 2.083 0.006 0.141
REMARK 2_xyz: 2.186 0.003 0.142
REMARK 2_adp: 2.186 0.003 0.142
REMARK 3_bss: 2.186 0.003 0.142
REMARK 3_xyz: 2.221 0.002 0.145
REMARK 3_adp: 2.221 0.002 0.145
REMARK 3_bss: 2.221 0.002 0.145
REMARK -------------------------------------------------------------
REMARK stage k1_w k1_t k3_w k3_t scale_ml
REMARK 0 : 0.3208 0.3361 0.3631 0.3758 1.0000
REMARK 1_bss: 0.4368 0.4308 0.4531 0.4522 1.0000
REMARK 1_xyz: 0.4385 0.4303 0.4529 0.4518 1.0000
REMARK 1_adp: 0.4317 0.4227 0.4454 0.4433 1.0000
REMARK 2_bss: 0.4388 0.4305 0.4528 0.4509 1.0000
REMARK 2_xyz: 0.4388 0.4315 0.4535 0.4519 1.0000
REMARK 2_sar: 0.4387 0.4314 0.4535 0.4518 1.0000
REMARK 2_xyz: 0.4390 0.4316 0.4536 0.4521 1.0000
REMARK 2_adp: 0.4332 0.4259 0.4474 0.4460 1.0000
REMARK 3_bss: 0.4384 0.4311 0.4528 0.4515 1.0000
REMARK 3_xyz: 0.4385 0.4314 0.4529 0.4518 1.0000
REMARK 3_adp: 0.4331 0.4258 0.4473 0.4460 1.0000
REMARK 3_bss: 0.4385 0.4313 0.4529 0.4517 1.0000
REMARK -------------------------------------------------------------
REMARK r_free_flags.md5.hexdigest 38c8444a6d884020b443671f38202fe9

TABLE A

| | | | | | | | |
|---|---|---|---|---|---|---|---|
| CRYST1 | 55.500 | 86.800 | 95.500 | 67.60 | 73.30 | 85.80 | P 1 |

| | | | | |
|---|---|---|---|---|
| SCALE1 | 0.018018 | −0.001323 | −0.005298 | 0.00000 |
| SCALE2 | 0.000000 | 0.011552 | −0.004700 | 0.00000 |
| SCALE3 | 0.000000 | 0.000000 | 0.011803 | 0.00000 |

| | | | | | | | | | | | |
|---|---|---|---|---|---|---|---|---|---|---|---|
| ATOM | 1 | N | TRP | A | 32 | 5.479 | 23.414 | 49.677 | 1.00 | 83.99 | N |
| ATOM | 2 | CA | TRP | A | 32 | 6.029 | 23.314 | 48.327 | 1.00 | 103.53 | C |
| ATOM | 3 | C | TRP | A | 32 | 5.622 | 24.515 | 47.517 | 1.00 | 92.41 | C |
| ATOM | 4 | O | TRP | A | 32 | 6.122 | 24.745 | 46.414 | 1.00 | 93.58 | O |
| ATOM | 5 | CB | TRP | A | 32 | 5.551 | 22.043 | 47.616 | 1.00 | 107.68 | C |
| ATOM | 6 | CG | TRP | A | 32 | 6.380 | 20.867 | 47.950 | 1.00 | 115.39 | C |
| ATOM | 7 | CD1 | TRP | A | 32 | 7.549 | 20.491 | 47.357 | 1.00 | 115.64 | C |
| ATOM | 8 | CD2 | TRP | A | 32 | 6.126 | 19.916 | 48.986 | 1.00 | 125.36 | C |
| ATOM | 9 | NE1 | TRP | A | 32 | 8.036 | 19.356 | 47.956 | 1.00 | 131.84 | N |
| ATOM | 10 | CE2 | TRP | A | 32 | 7.181 | 18.983 | 48.960 | 1.00 | 137.20 | C |
| ATOM | 11 | CE3 | TRP | A | 32 | 5.107 | 19.761 | 49.930 | 1.00 | 130.64 | C |
| ATOM | 12 | CZ2 | TRP | A | 32 | 7.243 | 17.905 | 49.844 | 1.00 | 144.93 | C |
| ATOM | 13 | CZ3 | TRP | A | 32 | 5.169 | 18.693 | 50.806 | 1.00 | 138.72 | C |
| ATOM | 14 | CH2 | TRP | A | 32 | 6.230 | 17.778 | 50.757 | 1.00 | 143.16 | C |
| ATOM | 15 | N | GLU | A | 33 | 4.700 | 25.286 | 48.066 | 1.00 | 75.79 | N |
| ATOM | 16 | CA | GLU | A | 33 | 4.178 | 26.417 | 47.327 | 1.00 | 71.94 | C |
| ATOM | 17 | C | GLU | A | 33 | 5.049 | 27.654 | 47.518 | 1.00 | 63.78 | C |
| ATOM | 18 | O | GLU | A | 33 | 5.368 | 28.347 | 46.553 | 1.00 | 57.27 | O |
| ATOM | 19 | CB | GLU | A | 33 | 2.739 | 26.700 | 47.718 | 1.00 | 62.61 | C |
| ATOM | 20 | CG | GLU | A | 33 | 2.043 | 27.620 | 46.749 | 1.00 | 80.74 | C |
| ATOM | 21 | CD | GLU | A | 33 | 0.731 | 28.138 | 47.287 | 1.00 | 97.56 | C |
| ATOM | 22 | OE1 | GLU | A | 33 | 0.304 | 27.692 | 48.375 | 1.00 | 98.25 | O |
| ATOM | 23 | OE2 | GLU | A | 33 | 0.128 | 28.998 | 46.621 | 1.00 | 93.05 | O |
| ATOM | 24 | N | ALA | A | 34 | 5.429 | 27.931 | 48.761 | 1.00 | 58.96 | N |
| ATOM | 25 | CA | ALA | A | 34 | 6.443 | 28.945 | 49.009 | 1.00 | 55.06 | C |
| ATOM | 26 | C | ALA | A | 34 | 7.704 | 28.540 | 48.253 | 1.00 | 53.77 | C |
| ATOM | 27 | O | ALA | A | 34 | 8.300 | 29.353 | 47.549 | 1.00 | 52.18 | O |
| ATOM | 28 | CB | ALA | A | 34 | 6.728 | 29.076 | 50.492 | 1.00 | 34.43 | C |
| ATOM | 29 | N | GLY | A | 35 | 8.089 | 27.273 | 48.390 | 1.00 | 47.25 | N |
| ATOM | 30 | CA | GLY | A | 35 | 9.239 | 26.738 | 47.682 | 1.00 | 48.47 | C |
| ATOM | 31 | C | GLY | A | 35 | 9.179 | 26.934 | 46.180 | 1.00 | 50.10 | C |
| ATOM | 32 | O | GLY | A | 35 | 10.011 | 27.627 | 45.597 | 1.00 | 43.83 | O |
| ATOM | 33 | N | MET | A | 36 | 8.184 | 26.333 | 45.541 | 1.00 | 58.69 | N |
| ATOM | 34 | CA | MET | A | 36 | 8.045 | 26.463 | 44.098 | 1.00 | 45.18 | C |
| ATOM | 35 | C | MET | A | 36 | 8.044 | 27.925 | 43.671 | 1.00 | 43.86 | C |
| ATOM | 36 | O | MET | A | 36 | 8.733 | 28.301 | 42.723 | 1.00 | 52.46 | O |
| ATOM | 37 | CB | MET | A | 36 | 6.803 | 25.724 | 43.597 | 1.00 | 50.39 | C |
| ATOM | 38 | CG | MET | A | 36 | 6.911 | 24.197 | 43.705 | 1.00 | 65.16 | C |
| ATOM | 39 | SD | MET | A | 36 | 8.354 | 23.460 | 42.883 | 1.00 | 75.80 | S |
| ATOM | 40 | CE | MET | A | 36 | 9.662 | 23.650 | 44.107 | 1.00 | 53.23 | C |
| ATOM | 41 | N | SER | A | 37 | 7.292 | 28.754 | 44.384 | 1.00 | 41.05 | N |
| ATOM | 42 | CA | SER | A | 37 | 7.224 | 30.183 | 44.073 | 1.00 | 42.61 | C |
| ATOM | 43 | C | SER | A | 37 | 8.597 | 30.850 | 44.051 | 1.00 | 39.66 | C |
| ATOM | 44 | O | SER | A | 37 | 8.904 | 31.646 | 43.162 | 1.00 | 27.74 | O |
| ATOM | 45 | CB | SER | A | 37 | 6.320 | 30.910 | 45.071 | 1.00 | 43.91 | C |
| ATOM | 46 | OG | SER | A | 37 | 4.961 | 30.572 | 44.865 | 1.00 | 49.57 | O |
| ATOM | 47 | N | LEU | A | 38 | 9.420 | 30.534 | 45.043 | 1.00 | 37.24 | N |
| ATOM | 48 | CA | LEU | A | 38 | 10.745 | 31.125 | 45.115 | 1.00 | 42.45 | C |
| ATOM | 49 | C | LEU | A | 38 | 11.632 | 30.645 | 43.953 | 1.00 | 44.56 | C |
| ATOM | 50 | O | LEU | A | 38 | 12.216 | 31.458 | 43.225 | 1.00 | 36.02 | O |
| ATOM | 51 | CB | LEU | A | 38 | 11.394 | 30.835 | 46.467 | 1.00 | 35.05 | C |
| ATOM | 52 | CG | LEU | A | 38 | 12.715 | 31.573 | 46.694 | 1.00 | 43.51 | C |
| ATOM | 53 | CD1 | LEU | A | 38 | 12.507 | 33.088 | 46.715 | 1.00 | 34.04 | C |
| ATOM | 54 | CD2 | LEU | A | 38 | 13.369 | 31.098 | 47.974 | 1.00 | 36.94 | C |
| ATOM | 55 | N | LEU | A | 39 | 11.716 | 29.329 | 43.774 | 1.00 | 37.17 | N |
| ATOM | 56 | CA | LEU | A | 39 | 12.446 | 28.768 | 42.643 | 1.00 | 42.97 | C |
| ATOM | 57 | C | LEU | A | 39 | 12.100 | 29.478 | 41.323 | 1.00 | 41.17 | C |
| ATOM | 58 | O | LEU | A | 39 | 12.989 | 29.831 | 40.547 | 1.00 | 38.80 | O |
| ATOM | 59 | CB | LEU | A | 39 | 12.195 | 27.263 | 42.518 | 1.00 | 42.81 | C |
| ATOM | 60 | CG | LEU | A | 39 | 12.958 | 26.578 | 41.380 | 1.00 | 42.86 | C |
| ATOM | 61 | CD1 | LEU | A | 39 | 14.458 | 26.501 | 41.687 | 1.00 | 38.86 | C |
| ATOM | 62 | CD2 | LEU | A | 39 | 12.381 | 25.194 | 41.098 | 1.00 | 49.27 | C |
| ATOM | 63 | N | MET | A | 40 | 10.810 | 29.692 | 41.076 | 1.00 | 37.62 | N |
| ATOM | 64 | CA | MET | A | 40 | 10.368 | 30.354 | 39.843 | 1.00 | 33.30 | C |
| ATOM | 65 | C | MET | A | 40 | 10.811 | 31.822 | 39.739 | 1.00 | 35.57 | C |
| ATOM | 66 | O | MET | A | 40 | 11.224 | 32.277 | 38.678 | 1.00 | 29.74 | O |
| ATOM | 67 | CB | MET | A | 40 | 8.850 | 30.223 | 39.667 | 1.00 | 38.00 | C |
| ATOM | 68 | CG | MET | A | 40 | 8.360 | 28.785 | 39.457 | 1.00 | 57.02 | C |
| ATOM | 69 | SD | MET | A | 40 | 9.045 | 27.919 | 38.005 | 1.00 | 83.10 | S |
| ATOM | 70 | CE | MET | A | 40 | 10.548 | 27.189 | 38.673 | 1.00 | 47.31 | C |
| ATOM | 71 | N | ALA | A | 41 | 10.740 | 32.564 | 40.839 | 1.00 | 42.39 | N |
| ATOM | 72 | CA | ALA | A | 41 | 11.243 | 33.931 | 40.838 | 1.00 | 32.14 | C |
| ATOM | 73 | C | ALA | A | 41 | 12.771 | 33.936 | 40.715 | 1.00 | 37.71 | C |
| ATOM | 74 | O | ALA | A | 41 | 13.378 | 34.983 | 40.503 | 1.00 | 34.34 | O |
| ATOM | 75 | CB | ALA | A | 41 | 10.814 | 34.647 | 42.102 | 1.00 | 38.26 | C |
| ATOM | 76 | N | LEU | A | 42 | 13.379 | 32.758 | 40.835 | 1.00 | 32.06 | N |
| ATOM | 77 | CA | LEU | A | 42 | 14.835 | 32.630 | 40.852 | 1.00 | 38.43 | C |
| ATOM | 78 | C | LEU | A | 42 | 15.479 | 31.951 | 39.621 | 1.00 | 37.50 | C |
| ATOM | 79 | O | LEU | A | 42 | 16.701 | 32.057 | 39.448 | 1.00 | 33.31 | O |
| ATOM | 80 | CB | LEU | A | 42 | 15.284 | 31.911 | 42.142 | 1.00 | 42.24 | C |
| ATOM | 81 | CG | LEU | A | 42 | 16.054 | 32.627 | 43.266 | 1.00 | 30.07 | C |
| ATOM | 82 | CD1 | LEU | A | 42 | 15.917 | 34.119 | 43.213 | 1.00 | 27.33 | C |
| ATOM | 83 | CD2 | LEU | A | 42 | 15.628 | 32.124 | 44.613 | 1.00 | 23.94 | C |
| ATOM | 84 | N | VAL | A | 43 | 14.685 | 31.268 | 38.779 | 1.00 | 30.39 | N |
| ATOM | 85 | CA | VAL | A | 43 | 15.249 | 30.423 | 37.688 | 1.00 | 31.64 | C |
| ATOM | 86 | C | VAL | A | 43 | 16.172 | 31.090 | 36.674 | 1.00 | 30.23 | C |
| ATOM | 87 | O | VAL | A | 43 | 17.251 | 30.580 | 36.414 | 1.00 | 34.48 | O |
| ATOM | 88 | CB | VAL | A | 43 | 14.196 | 29.668 | 36.847 | 1.00 | 28.23 | C |
| ATOM | 89 | CG1 | VAL | A | 43 | 14.255 | 28.170 | 37.121 | 1.00 | 31.36 | C |
| ATOM | 90 | CG2 | VAL | A | 43 | 12.806 | 30.254 | 37.029 | 1.00 | 43.70 | C |
| ATOM | 91 | N | VAL | A | 44 | 15.735 | 32.183 | 36.058 | 1.00 | 25.45 | N |
| ATOM | 92 | CA | VAL | A | 44 | 16.586 | 32.861 | 35.093 | 1.00 | 25.39 | C |
| ATOM | 93 | C | VAL | A | 44 | 17.953 | 33.133 | 35.693 | 1.00 | 31.88 | C |
| ATOM | 94 | O | VAL | A | 44 | 18.972 | 32.858 | 35.063 | 1.00 | 36.72 | O |
| ATOM | 95 | CB | VAL | A | 44 | 15.980 | 34.173 | 34.582 | 1.00 | 33.02 | C |
| ATOM | 96 | CG1 | VAL | A | 44 | 16.977 | 34.908 | 33.706 | 1.00 | 20.74 | C |
| ATOM | 97 | CG2 | VAL | A | 44 | 14.697 | 33.896 | 33.819 | 1.00 | 29.01 | C |
| ATOM | 98 | N | LEU | A | 45 | 17.972 | 33.649 | 36.919 | 1.00 | 34.27 | N |
| ATOM | 99 | CA | LEU | A | 45 | 19.226 | 33.872 | 37.631 | 1.00 | 34.28 | C |
| ATOM | 100 | C | LEU | A | 45 | 19.967 | 32.552 | 37.856 | 1.00 | 33.96 | C |
| ATOM | 101 | O | LEU | A | 45 | 21.116 | 32.404 | 37.434 | 1.00 | 35.46 | O |
| ATOM | 102 | CB | LEU | A | 45 | 18.991 | 34.589 | 38.962 | 1.00 | 25.07 | C |
| ATOM | 103 | CG | LEU | A | 45 | 20.249 | 34.728 | 39.828 | 1.00 | 35.24 | C |
| ATOM | 104 | CD1 | LEU | A | 45 | 21.202 | 35.825 | 39.313 | 1.00 | 24.13 | C |
| ATOM | 105 | CD2 | LEU | A | 45 | 19.870 | 34.972 | 41.275 | 1.00 | 34.92 | C |
| ATOM | 106 | N | LEU | A | 46 | 19.299 | 31.600 | 38.505 | 1.00 | 29.50 | N |
| ATOM | 107 | CA | LEU | A | 46 | 19.877 | 30.279 | 38.797 | 1.00 | 34.70 | C |
| ATOM | 108 | C | LEU | A | 46 | 20.472 | 29.516 | 37.598 | 1.00 | 36.81 | C |
| ATOM | 109 | O | LEU | A | 46 | 21.490 | 28.835 | 37.730 | 1.00 | 39.64 | O |
| ATOM | 110 | CB | LEU | A | 46 | 18.841 | 29.386 | 39.479 | 1.00 | 33.97 | C |
| ATOM | 111 | CG | LEU | A | 46 | 18.535 | 29.691 | 40.938 | 1.00 | 37.67 | C |
| ATOM | 112 | CD1 | LEU | A | 46 | 17.621 | 28.615 | 41.508 | 1.00 | 38.94 | C |
| ATOM | 113 | CD2 | LEU | A | 46 | 19.832 | 29.762 | 41.698 | 1.00 | 28.47 | C |
| ATOM | 114 | N | ILE | A | 47 | 19.821 | 29.606 | 36.443 | 1.00 | 33.68 | N |
| ATOM | 115 | CA | ILE | A | 47 | 20.318 | 28.953 | 35.236 | 1.00 | 37.11 | C |
| ATOM | 116 | C | ILE | A | 47 | 21.516 | 29.703 | 34.651 | 1.00 | 35.09 | C |
| ATOM | 117 | O | ILE | A | 47 | 22.527 | 29.088 | 34.333 | 1.00 | 31.56 | O |
| ATOM | 118 | CB | ILE | A | 47 | 19.215 | 28.815 | 34.144 | 1.00 | 34.69 | C |
| ATOM | 119 | CG1 | ILE | A | 47 | 18.103 | 27.868 | 34.593 | 1.00 | 33.71 | C |
| ATOM | 120 | CG2 | ILE | A | 47 | 19.804 | 28.309 | 32.857 | 1.00 | 26.83 | C |
| ATOM | 121 | CD1 | ILE | A | 47 | 16.810 | 27.998 | 33.786 | 1.00 | 24.38 | C |
| ATOM | 122 | N | VAL | A | 48 | 21.395 | 31.026 | 34.518 | 1.00 | 36.00 | N |
| ATOM | 123 | CA | VAL | A | 48 | 22.413 | 31.842 | 33.857 | 1.00 | 29.90 | C |
| ATOM | 124 | C | VAL | A | 48 | 23.676 | 32.035 | 34.684 | 1.00 | 33.15 | C |
| ATOM | 125 | O | VAL | A | 48 | 24.773 | 31.740 | 34.214 | 1.00 | 41.11 | O |
| ATOM | 126 | CB | VAL | A | 48 | 21.877 | 33.226 | 33.428 | 1.00 | 29.31 | C |
| ATOM | 127 | CG1 | VAL | A | 48 | 23.017 | 34.123 | 32.978 | 1.00 | 21.44 | C |
| ATOM | 128 | CG2 | VAL | A | 48 | 20.864 | 33.080 | 32.317 | 1.00 | 29.53 | C |
| ATOM | 129 | N | ALA | A | 49 | 23.533 | 32.542 | 35.902 | 1.00 | 33.37 | N |
| ATOM | 130 | CA | ALA | A | 49 | 24.692 | 32.758 | 36.768 | 1.00 | 38.03 | C |
| ATOM | 131 | C | ALA | A | 49 | 25.399 | 31.441 | 37.089 | 1.00 | 40.97 | C |
| ATOM | 132 | O | ALA | A | 49 | 26.626 | 31.388 | 37.164 | 1.00 | 37.86 | O |
| ATOM | 133 | CB | ALA | A | 49 | 24.278 | 33.457 | 38.047 | 1.00 | 30.38 | C |
| ATOM | 134 | N | GLY | A | 50 | 24.614 | 30.383 | 37.277 | 1.00 | 40.04 | N |
| ATOM | 135 | CA | GLY | A | 50 | 25.143 | 29.083 | 37.650 | 1.00 | 33.14 | C |
| ATOM | 136 | C | GLY | A | 50 | 25.866 | 28.360 | 36.524 | 1.00 | 40.38 | C |
| ATOM | 137 | O | GLY | A | 50 | 26.825 | 27.625 | 36.758 | 1.00 | 40.26 | O |
| ATOM | 138 | N | ASN | A | 51 | 25.408 | 28.545 | 35.292 | 1.00 | 31.96 | N |
| ATOM | 139 | CA | ASN | A | 51 | 26.053 | 27.877 | 34.175 | 1.00 | 32.47 | C |
| ATOM | 140 | C | ASN | A | 51 | 27.200 | 28.705 | 33.643 | 1.00 | 32.88 | C |
| ATOM | 141 | O | ASN | A | 51 | 28.197 | 28.165 | 33.191 | 1.00 | 33.20 | O |
| ATOM | 142 | CB | ASN | A | 51 | 25.051 | 27.541 | 33.080 | 1.00 | 30.53 | C |
| ATOM | 143 | CG | ASN | A | 51 | 24.223 | 26.313 | 33.417 | 1.00 | 37.29 | C |
| ATOM | 144 | OD1 | ASN | A | 51 | 24.649 | 25.173 | 33.188 | 1.00 | 28.91 | O |
| ATOM | 145 | ND2 | ASN | A | 51 | 23.026 | 26.540 | 33.963 | 1.00 | 28.81 | N |
| ATOM | 146 | N | VAL | A | 52 | 27.055 | 30.023 | 33.706 | 1.00 | 33.30 | N |
| ATOM | 147 | CA | VAL | A | 52 | 28.163 | 30.912 | 33.423 | 1.00 | 30.80 | C |
| ATOM | 148 | C | VAL | A | 52 | 29.286 | 30.565 | 34.390 | 1.00 | 43.40 | C |
| ATOM | 149 | O | VAL | A | 52 | 30.469 | 30.618 | 34.045 | 1.00 | 35.89 | O |
| ATOM | 150 | CB | VAL | A | 52 | 27.779 | 32.377 | 33.614 | 1.00 | 30.90 | C |
| ATOM | 151 | CG1 | VAL | A | 52 | 29.020 | 33.215 | 33.845 | 1.00 | 20.60 | C |
| ATOM | 152 | CG2 | VAL | A | 52 | 27.002 | 32.884 | 32.406 | 1.00 | 33.55 | C |
| ATOM | 153 | N | LEU | A | 53 | 28.901 | 30.175 | 35.600 | 1.00 | 42.58 | N |
| ATOM | 154 | CA | LEU | A | 53 | 29.864 | 29.853 | 36.642 | 1.00 | 39.51 | C |
| ATOM | 155 | C | LEU | A | 53 | 30.575 | 28.520 | 36.399 | 1.00 | 44.56 | C |
| ATOM | 156 | O | LEU | A | 53 | 31.778 | 28.407 | 36.628 | 1.00 | 42.77 | O |
| ATOM | 157 | CB | LEU | A | 53 | 29.175 | 29.850 | 38.001 | 1.00 | 40.87 | C |
| ATOM | 158 | CG | LEU | A | 53 | 29.978 | 30.484 | 39.139 | 1.00 | 71.27 | C |
| ATOM | 159 | CD1 | LEU | A | 53 | 30.315 | 31.943 | 38.829 | 1.00 | 53.98 | C |
| ATOM | 160 | CD2 | LEU | A | 53 | 29.205 | 30.373 | 40.440 | 1.00 | 80.71 | C |
| ATOM | 161 | N | VAL | A | 54 | 29.831 | 27.514 | 35.940 | 1.00 | 45.68 | N |
| ATOM | 162 | CA | VAL | A | 54 | 30.419 | 26.222 | 35.584 | 1.00 | 40.52 | C |
| ATOM | 163 | C | VAL | A | 54 | 31.416 | 26.409 | 34.441 | 1.00 | 42.52 | C |
| ATOM | 164 | O | VAL | A | 54 | 32.523 | 25.873 | 34.458 | 1.00 | 40.69 | O |
| ATOM | 165 | CB | VAL | A | 54 | 29.338 | 25.203 | 35.145 | 1.00 | 40.15 | C |
| ATOM | 166 | CG1 | VAL | A | 54 | 29.970 | 24.032 | 34.394 | 1.00 | 32.03 | C |
| ATOM | 167 | CG2 | VAL | A | 54 | 28.533 | 24.712 | 36.337 | 1.00 | 33.68 | C |
| ATOM | 168 | N | ILE | A | 55 | 31.009 | 27.181 | 33.444 | 1.00 | 39.95 | N |
| ATOM | 169 | CA | ILE | A | 55 | 31.864 | 27.461 | 32.306 | 1.00 | 44.35 | C |
| ATOM | 170 | C | ILE | A | 55 | 33.167 | 28.125 | 32.762 | 1.00 | 51.31 | C |
| ATOM | 171 | O | ILE | A | 55 | 34.245 | 27.759 | 32.306 | 1.00 | 60.77 | O |
| ATOM | 172 | CB | ILE | A | 55 | 31.124 | 28.318 | 31.249 | 1.00 | 36.78 | C |
| ATOM | 173 | CG1 | ILE | A | 55 | 30.298 | 27.421 | 30.324 | 1.00 | 30.86 | C |
| ATOM | 174 | CG2 | ILE | A | 55 | 32.092 | 29.161 | 30.447 | 1.00 | 23.46 | C |
| ATOM | 175 | CD1 | ILE | A | 55 | 29.288 | 28.185 | 29.492 | 1.00 | 32.30 | C |
| ATOM | 176 | N | ALA | A | 56 | 33.073 | 29.079 | 33.680 | 1.00 | 44.33 | N |
| ATOM | 177 | CA | ALA | A | 56 | 34.260 | 29.803 | 34.109 | 1.00 | 40.24 | C |
| ATOM | 178 | C | ALA | A | 56 | 35.166 | 28.941 | 34.985 | 1.00 | 44.67 | C |
| ATOM | 179 | O | ALA | A | 56 | 36.382 | 29.020 | 34.887 | 1.00 | 56.47 | O |
| ATOM | 180 | CB | ALA | A | 56 | 33.879 | 31.093 | 34.825 | 1.00 | 41.62 | C |
| ATOM | 181 | N | ALA | A | 57 | 34.576 | 28.122 | 35.845 | 1.00 | 41.10 | N |
| ATOM | 182 | CA | ALA | A | 57 | 35.360 | 27.233 | 36.692 | 1.00 | 46.52 | C |
| ATOM | 183 | C | ALA | A | 57 | 36.164 | 26.255 | 35.842 | 1.00 | 51.89 | C |
| ATOM | 184 | O | ALA | A | 57 | 37.365 | 26.086 | 36.036 | 1.00 | 56.20 | O |
| ATOM | 185 | CB | ALA | A | 57 | 34.456 | 26.475 | 37.664 | 1.00 | 38.14 | C |
| ATOM | 186 | N | ILE | A | 58 | 35.489 | 25.609 | 34.898 | 1.00 | 50.72 | N |
| ATOM | 187 | CA | ILE | A | 58 | 36.141 | 24.662 | 34.007 | 1.00 | 55.77 | C |
| ATOM | 188 | C | ILE | A | 58 | 37.234 | 25.356 | 33.203 | 1.00 | 52.34 | C |
| ATOM | 189 | O | ILE | A | 58 | 38.212 | 24.732 | 32.800 | 1.00 | 69.28 | O |
| ATOM | 190 | CB | ILE | A | 58 | 35.126 | 24.000 | 33.045 | 1.00 | 50.04 | C |
| ATOM | 191 | CG1 | ILE | A | 58 | 34.268 | 22.972 | 33.787 | 1.00 | 41.61 | C |
| ATOM | 192 | CG2 | ILE | A | 58 | 35.833 | 23.333 | 31.881 | 1.00 | 44.93 | C |
| ATOM | 193 | CD1 | ILE | A | 58 | 33.148 | 22.407 | 32.941 | 1.00 | 29.39 | C |
| ATOM | 194 | N | GLY | A | 59 | 37.069 | 26.654 | 32.983 | 1.00 | 47.92 | N |
| ATOM | 195 | CA | GLY | A | 59 | 38.017 | 27.423 | 32.197 | 1.00 | 54.58 | C |
| ATOM | 196 | C | GLY | A | 59 | 39.134 | 28.073 | 33.000 | 1.00 | 57.00 | C |
| ATOM | 197 | O | GLY | A | 59 | 40.032 | 28.681 | 32.432 | 1.00 | 57.28 | O |
| ATOM | 198 | N | SER | A | 60 | 39.079 | 27.956 | 34.321 | 1.00 | 52.25 | N |
| ATOM | 199 | CA | SER | A | 60 | 40.149 | 28.459 | 35.171 | 1.00 | 52.71 | C |
| ATOM | 200 | C | SER | A | 60 | 41.058 | 27.304 | 35.543 | 1.00 | 68.82 | C |
| ATOM | 201 | O | SER | A | 60 | 42.132 | 27.132 | 34.964 | 1.00 | 90.28 | O |
| ATOM | 202 | CB | SER | A | 60 | 39.589 | 29.097 | 36.447 | 1.00 | 65.02 | C |
| ATOM | 203 | OG | SER | A | 60 | 39.016 | 30.368 | 36.193 | 1.00 | 65.89 | O |
| ATOM | 204 | N | THR | A | 61 | 40.613 | 26.512 | 36.514 | 1.00 | 67.59 | N |
| ATOM | 205 | CA | THR | A | 61 | 41.335 | 25.325 | 36.955 | 1.00 | 71.80 | C |
| ATOM | 206 | C | THR | A | 61 | 41.599 | 24.378 | 35.797 | 1.00 | 82.94 | C |
| ATOM | 207 | O | THR | A | 61 | 40.694 | 23.663 | 35.364 | 1.00 | 82.55 | O |
| ATOM | 208 | CB | THR | A | 61 | 40.516 | 24.522 | 37.976 | 1.00 | 64.49 | C |
| ATOM | 209 | OG1 | THR | A | 61 | 39.657 | 25.398 | 38.712 | 1.00 | 65.85 | O |
| ATOM | 210 | CG2 | THR | A | 61 | 41.438 | 23.769 | 38.923 | 1.00 | 79.54 | C |
| ATOM | 211 | N | GLN | A | 62 | 42.833 | 24.364 | 35.301 | 1.00 | 92.97 | N |
| ATOM | 212 | CA | GLN | A | 62 | 43.225 | 23.405 | 34.273 | 1.00 | 99.18 | C |
| ATOM | 213 | C | GLN | A | 62 | 43.106 | 21.999 | 34.853 | 1.00 | 95.18 | C |
| ATOM | 214 | O | GLN | A | 62 | 42.926 | 21.016 | 34.124 | 1.00 | 88.85 | O |
| ATOM | 215 | CB | GLN | A | 62 | 44.648 | 23.696 | 33.797 | 1.00 | 107.65 | C |
| ATOM | 216 | CG | GLN | A | 62 | 44.767 | 25.037 | 33.086 | 1.00 | 118.47 | C |
| ATOM | 217 | CD | GLN | A | 62 | 46.074 | 25.741 | 33.371 | 1.00 | 138.62 | C |
| ATOM | 218 | OE1 | GLN | A | 62 | 47.106 | 25.102 | 33.583 | 1.00 | 149.46 | O |
| ATOM | 219 | NE2 | GLN | A | 62 | 46.037 | 27.071 | 33.382 | 1.00 | 136.58 | N |
| ATOM | 220 | N | ARG | A | 63 | 43.192 | 21.933 | 36.179 | 1.00 | 73.75 | N |
| ATOM | 221 | CA | ARG | A | 63 | 42.943 | 20.720 | 36.940 | 1.00 | 67.13 | C |
| ATOM | 222 | C | ARG | A | 63 | 41.479 | 20.264 | 36.808 | 1.00 | 80.88 | C |
| ATOM | 223 | O | ARG | A | 63 | 41.130 | 19.135 | 37.158 | 1.00 | 78.75 | O |
| ATOM | 224 | CB | ARG | A | 63 | 43.288 | 20.986 | 38.400 | 1.00 | 85.45 | C |
| ATOM | 225 | CG | ARG | A | 63 | 42.782 | 19.954 | 39.377 | 1.00 | 97.30 | C |
| ATOM | 226 | CD | ARG | A | 63 | 42.420 | 20.622 | 40.683 | 1.00 | 103.16 | C |
| ATOM | 227 | NE | ARG | A | 63 | 42.561 | 19.729 | 41.827 | 1.00 | 120.92 | N |
| ATOM | 228 | CZ | ARG | A | 63 | 42.028 | 19.966 | 43.022 | 1.00 | 126.50 | C |
| ATOM | 229 | NH1 | ARG | A | 63 | 41.305 | 21.064 | 43.220 | 1.00 | 114.21 | N |
| ATOM | 230 | NH2 | ARG | A | 63 | 42.208 | 19.104 | 44.015 | 1.00 | 122.66 | N |
| ATOM | 231 | N | LEU | A | 64 | 40.624 | 21.158 | 36.316 | 1.00 | 81.30 | N |
| ATOM | 232 | CA | LEU | A | 64 | 39.252 | 20.813 | 35.948 | 1.00 | 64.33 | C |
| ATOM | 233 | C | LEU | A | 64 | 39.121 | 20.686 | 34.430 | 1.00 | 68.67 | C |
| ATOM | 234 | O | LEU | A | 64 | 38.023 | 20.521 | 33.907 | 1.00 | 58.81 | O |
| ATOM | 235 | CB | LEU | A | 64 | 38.269 | 21.874 | 36.439 | 1.00 | 62.31 | C |
| ATOM | 236 | CG | LEU | A | 64 | 37.609 | 21.730 | 37.812 | 1.00 | 61.10 | C |
| ATOM | 237 | CD1 | LEU | A | 64 | 36.568 | 22.830 | 37.993 | 1.00 | 51.33 | C |
| ATOM | 238 | CD2 | LEU | A | 64 | 36.987 | 20.355 | 37.998 | 1.00 | 42.50 | C |
| ATOM | 239 | N | GLN | A | 65 | 40.239 | 20.779 | 33.718 | 1.00 | 73.78 | N |
| ATOM | 240 | CA | GLN | A | 65 | 40.206 | 20.632 | 32.269 | 1.00 | 81.50 | C |
| ATOM | 241 | C | GLN | A | 65 | 40.478 | 19.193 | 31.841 | 1.00 | 74.21 | C |
| ATOM | 242 | O | GLN | A | 65 | 41.621 | 18.739 | 31.814 | 1.00 | 69.96 | O |
| ATOM | 243 | CB | GLN | A | 65 | 41.149 | 21.625 | 31.586 | 1.00 | 76.94 | C |
| ATOM | 244 | CG | GLN | A | 65 | 40.470 | 22.945 | 31.258 | 1.00 | 77.65 | C |
| ATOM | 245 | CD | GLN | A | 65 | 41.430 | 23.980 | 30.721 | 1.00 | 98.72 | C |
| ATOM | 246 | OE1 | GLN | A | 65 | 42.560 | 24.091 | 31.193 | 1.00 | 98.33 | O |
| ATOM | 247 | NE2 | GLN | A | 65 | 40.983 | 24.752 | 29.731 | 1.00 | 96.05 | N |
| ATOM | 248 | N | THR | A | 66 | 39.398 | 18.482 | 31.529 | 1.00 | 63.49 | N |
| ATOM | 249 | CA | THR | A | 66 | 39.456 | 17.080 | 31.150 | 1.00 | 51.39 | C |
| ATOM | 250 | C | THR | A | 66 | 38.438 | 16.838 | 30.041 | 1.00 | 44.86 | C |
| ATOM | 251 | O | THR | A | 66 | 37.529 | 17.639 | 29.856 | 1.00 | 44.94 | O |
| ATOM | 252 | CB | THR | A | 66 | 39.151 | 16.162 | 32.361 | 1.00 | 52.62 | C |
| ATOM | 253 | OG1 | THR | A | 66 | 37.787 | 16.325 | 32.773 | 1.00 | 45.47 | O |
| ATOM | 254 | CG2 | THR | A | 66 | 40.053 | 16.508 | 33.529 | 1.00 | 46.17 | C |
| ATOM | 255 | N | LEU | A | 67 | 38.601 | 15.750 | 29.295 | 1.00 | 46.24 | N |
| ATOM | 256 | CA | LEU | A | 67 | 37.660 | 15.390 | 28.238 | 1.00 | 42.57 | C |
| ATOM | 257 | C | LEU | A | 67 | 36.206 | 15.476 | 28.693 | 1.00 | 47.62 | C |
| ATOM | 258 | O | LEU | A | 67 | 35.410 | 16.219 | 28.116 | 1.00 | 40.50 | O |
| ATOM | 259 | CB | LEU | A | 67 | 37.936 | 13.971 | 27.746 | 1.00 | 55.94 | C |
| ATOM | 260 | CG | LEU | A | 67 | 39.184 | 13.828 | 26.886 | 1.00 | 47.72 | C |
| ATOM | 261 | CD1 | LEU | A | 67 | 39.151 | 12.502 | 26.157 | 1.00 | 62.62 | C |
| ATOM | 262 | CD2 | LEU | A | 67 | 39.241 | 14.978 | 25.910 | 1.00 | 43.96 | C |
| ATOM | 263 | N | THR | A | 68 | 35.864 | 14.701 | 29.721 | 1.00 | 36.53 | N |
| ATOM | 264 | CA | THR | A | 68 | 34.515 | 14.707 | 30.266 | 1.00 | 40.04 | C |
| ATOM | 265 | C | THR | A | 68 | 33.985 | 16.134 | 30.490 | 1.00 | 35.56 | C |
| ATOM | 266 | O | THR | A | 68 | 32.825 | 16.420 | 30.204 | 1.00 | 42.74 | O |
| ATOM | 267 | CB | THR | A | 68 | 34.415 | 13.870 | 31.573 | 1.00 | 36.84 | C |
| ATOM | 268 | OG1 | THR | A | 68 | 34.619 | 12.485 | 31.279 | 1.00 | 36.37 | O |
| ATOM | 269 | CG2 | THR | A | 68 | 33.051 | 14.018 | 32.205 | 1.00 | 37.20 | C |
| ATOM | 270 | N | ASN | A | 69 | 34.835 | 17.032 | 30.973 | 1.00 | 34.20 | N |
| ATOM | 271 | CA | ASN | A | 69 | 34.406 | 18.402 | 31.285 | 1.00 | 35.19 | C |
| ATOM | 272 | C | ASN | A | 69 | 34.169 | 19.306 | 30.074 | 1.00 | 34.58 | C |
| ATOM | 273 | O | ASN | A | 69 | 33.473 | 20.315 | 30.179 | 1.00 | 31.28 | O |
| ATOM | 274 | CB | ASN | A | 69 | 35.366 | 19.066 | 32.276 | 1.00 | 35.89 | C |
| ATOM | 275 | CG | ASN | A | 69 | 35.169 | 18.564 | 33.690 | 1.00 | 53.27 | C |
| ATOM | 276 | OD1 | ASN | A | 69 | 34.115 | 18.010 | 34.014 | 1.00 | 48.94 | O |
| ATOM | 277 | ND2 | ASN | A | 69 | 36.179 | 18.742 | 34.542 | 1.00 | 58.63 | N |
| ATOM | 278 | N | LEU | A | 70 | 34.752 | 18.946 | 28.933 | 1.00 | 34.78 | N |
| ATOM | 279 | CA | LEU | A | 70 | 34.447 | 19.612 | 27.674 | 1.00 | 31.35 | C |
| ATOM | 280 | C | LEU | A | 70 | 32.981 | 19.354 | 27.300 | 1.00 | 33.12 | C |
| ATOM | 281 | O | LEU | A | 70 | 32.251 | 20.263 | 26.900 | 1.00 | 26.98 | O |
| ATOM | 282 | CB | LEU | A | 70 | 35.370 | 19.100 | 26.574 | 1.00 | 38.11 | C |
| ATOM | 283 | CG | LEU | A | 70 | 36.710 | 19.804 | 26.387 | 1.00 | 34.46 | C |
| ATOM | 284 | CD1 | LEU | A | 70 | 37.517 | 19.192 | 25.216 | 1.00 | 30.63 | C |
| ATOM | 285 | CD2 | LEU | A | 70 | 36.437 | 21.273 | 26.155 | 1.00 | 37.05 | C |
| ATOM | 286 | N | PHE | A | 71 | 32.556 | 18.106 | 27.444 | 1.00 | 27.08 | N |
| ATOM | 287 | CA | PHE | A | 71 | 31.167 | 17.749 | 27.248 | 1.00 | 29.77 | C |
| ATOM | 288 | C | PHE | A | 71 | 30.261 | 18.483 | 28.229 | 1.00 | 34.66 | C |
| ATOM | 289 | O | PHE | A | 71 | 29.180 | 18.945 | 27.854 | 1.00 | 36.20 | O |
| ATOM | 290 | CB | PHE | A | 71 | 30.979 | 16.244 | 27.396 | 1.00 | 36.47 | C |
| ATOM | 291 | CG | PHE | A | 71 | 31.766 | 15.439 | 26.402 | 1.00 | 41.69 | C |
| ATOM | 292 | CD1 | PHE | A | 71 | 32.001 | 15.933 | 25.131 | 1.00 | 33.87 | C |
| ATOM | 293 | CD2 | PHE | A | 71 | 32.261 | 14.184 | 26.732 | 1.00 | 39.10 | C |
| ATOM | 294 | CE1 | PHE | A | 71 | 32.717 | 15.194 | 24.215 | 1.00 | 35.55 | C |
| ATOM | 295 | CE2 | PHE | A | 71 | 32.981 | 13.448 | 25.815 | 1.00 | 35.39 | C |
| ATOM | 296 | CZ | PHE | A | 71 | 33.211 | 13.953 | 24.559 | 1.00 | 28.51 | C |
| ATOM | 297 | N | ILE | A | 72 | 30.700 | 18.583 | 29.482 | 1.00 | 31.33 | N |
| ATOM | 298 | CA | ILE | A | 72 | 29.966 | 19.322 | 30.509 | 1.00 | 28.07 | C |
| ATOM | 299 | C | ILE | A | 72 | 29.721 | 20.765 | 30.064 | 1.00 | 29.37 | C |
| ATOM | 300 | O | ILE | A | 72 | 28.672 | 21.346 | 30.328 | 1.00 | 33.16 | O |
| ATOM | 301 | CB | ILE | A | 72 | 30.729 | 19.320 | 31.845 | 1.00 | 32.56 | C |
| ATOM | 302 | CG1 | ILE | A | 72 | 30.720 | 17.924 | 32.472 | 1.00 | 30.68 | C |
| ATOM | 303 | CG2 | ILE | A | 72 | 30.155 | 20.350 | 32.806 | 1.00 | 32.38 | C |
| ATOM | 304 | CD1 | ILE | A | 72 | 29.363 | 17.339 | 32.658 | 1.00 | 23.38 | C |
| ATOM | 305 | N | THR | A | 73 | 30.702 | 21.327 | 29.375 | 1.00 | 29.77 | N |
| ATOM | 306 | CA | THR | A | 73 | 30.635 | 22.697 | 28.891 | 1.00 | 34.60 | C |
| ATOM | 307 | C | THR | A | 73 | 29.638 | 22.856 | 27.758 | 1.00 | 35.71 | C |
| ATOM | 308 | O | THR | A | 73 | 29.046 | 23.923 | 27.584 | 1.00 | 40.54 | O |
| ATOM | 309 | CB | THR | A | 73 | 32.014 | 23.171 | 28.394 | 1.00 | 27.39 | C |
| ATOM | 310 | OG1 | THR | A | 73 | 32.973 | 22.984 | 29.435 | 1.00 | 25.98 | O |
| ATOM | 311 | CG2 | THR | A | 73 | 31.975 | 24.648 | 27.998 | 1.00 | 17.40 | C |
| ATOM | 312 | N | SER | A | 74 | 29.486 | 21.803 | 26.964 | 1.00 | 32.75 | N |
| ATOM | 313 | CA | SER | A | 74 | 28.515 | 21.799 | 25.882 | 1.00 | 32.03 | C |
| ATOM | 314 | C | SER | A | 74 | 27.156 | 21.812 | 26.559 | 1.00 | 34.14 | C |
| ATOM | 315 | O | SER | A | 74 | 26.260 | 22.585 | 26.198 | 1.00 | 31.27 | O |
| ATOM | 316 | CB | SER | A | 74 | 28.693 | 20.540 | 25.025 | 1.00 | 31.36 | C |
| ATOM | 317 | OG | SER | A | 74 | 27.742 | 20.462 | 23.978 | 1.00 | 38.64 | O |
| ATOM | 318 | N | LEU | A | 75 | 27.039 | 20.960 | 27.574 | 1.00 | 30.55 | N |
| ATOM | 319 | CA | LEU | A | 75 | 25.827 | 20.814 | 28.368 | 1.00 | 26.60 | C |
| ATOM | 320 | C | LEU | A | 75 | 25.392 | 22.121 | 29.012 | 1.00 | 26.46 | C |
| ATOM | 321 | O | LEU | A | 75 | 24.198 | 22.390 | 29.129 | 1.00 | 28.93 | O |
| ATOM | 322 | CB | LEU | A | 75 | 26.047 | 19.766 | 29.446 | 1.00 | 24.73 | C |
| ATOM | 323 | CG | LEU | A | 75 | 24.782 | 19.050 | 29.891 | 1.00 | 27.34 | C |
| ATOM | 324 | CD1 | LEU | A | 75 | 23.774 | 19.026 | 28.757 | 1.00 | 20.89 | C |
| ATOM | 325 | CD2 | LEU | A | 75 | 25.132 | 17.642 | 30.357 | 1.00 | 27.17 | C |
| ATOM | 326 | N | ALA | A | 76 | 26.368 | 22.926 | 29.421 | 1.00 | 25.85 | N |
| ATOM | 327 | CA | ALA | A | 76 | 26.113 | 24.209 | 30.066 | 1.00 | 26.99 | C |
| ATOM | 328 | C | ALA | A | 76 | 25.709 | 25.314 | 29.081 | 1.00 | 28.89 | C |
| ATOM | 329 | O | ALA | A | 76 | 25.024 | 26.269 | 29.453 | 1.00 | 30.30 | O |
| ATOM | 330 | CB | ALA | A | 76 | 27.313 | 24.634 | 30.881 | 1.00 | 23.85 | C |
| ATOM | 331 | N | CYS | A | 77 | 26.130 | 25.189 | 27.828 | 1.00 | 29.63 | N |
| ATOM | 332 | CA | CYS | A | 77 | 25.719 | 26.144 | 26.809 | 1.00 | 32.36 | C |
| ATOM | 333 | C | CYS | A | 77 | 24.271 | 25.895 | 26.385 | 1.00 | 32.08 | C |
| ATOM | 334 | O | CYS | A | 77 | 23.494 | 26.836 | 26.199 | 1.00 | 27.57 | O |
| ATOM | 335 | CB | CYS | A | 77 | 26.659 | 26.092 | 25.614 | 1.00 | 25.58 | C |
| ATOM | 336 | SG | CYS | A | 77 | 28.277 | 26.736 | 26.014 | 1.00 | 43.24 | S |
| ATOM | 337 | N | ALA | A | 78 | 23.908 | 24.626 | 26.236 | 1.00 | 30.96 | N |
| ATOM | 338 | CA | ALA | A | 78 | 22.511 | 24.273 | 26.041 | 1.00 | 28.35 | C |
| ATOM | 339 | C | ALA | A | 78 | 21.709 | 24.998 | 27.103 | 1.00 | 30.79 | C |
| ATOM | 340 | O | ALA | A | 78 | 20.660 | 25.566 | 26.822 | 1.00 | 30.86 | O |
| ATOM | 341 | CB | ALA | A | 78 | 22.309 | 22.779 | 26.170 | 1.00 | 19.75 | C |
| ATOM | 342 | N | ASP | A | 79 | 22.233 | 24.996 | 28.327 | 1.00 | 32.81 | N |
| ATOM | 343 | CA | ASP | A | 79 | 21.539 | 25.577 | 29.475 | 1.00 | 27.05 | C |
| ATOM | 344 | C | ASP | A | 79 | 21.579 | 27.094 | 29.477 | 1.00 | 26.73 | C |
| ATOM | 345 | O | ASP | A | 79 | 20.596 | 27.745 | 29.817 | 1.00 | 26.51 | O |
| ATOM | 346 | CB | ASP | A | 79 | 22.100 | 25.022 | 30.774 | 1.00 | 23.51 | C |
| ATOM | 347 | CG | ASP | A | 79 | 21.485 | 23.686 | 31.147 | 1.00 | 34.58 | C |
| ATOM | 348 | OD1 | ASP | A | 79 | 20.690 | 23.150 | 30.349 | 1.00 | 36.21 | O |
| ATOM | 349 | OD2 | ASP | A | 79 | 21.799 | 23.167 | 32.245 | 1.00 | 56.57 | O |
| ATOM | 350 | N | LEU | A | 80 | 22.708 | 27.661 | 29.079 | 1.00 | 28.45 | N |
| ATOM | 351 | CA | LEU | A | 80 | 22.785 | 29.101 | 28.908 | 1.00 | 33.58 | C |
| ATOM | 352 | C | LEU | A | 80 | 21.723 | 29.607 | 27.912 | 1.00 | 37.54 | C |
| ATOM | 353 | O | LEU | A | 80 | 21.093 | 30.649 | 28.110 | 1.00 | 32.93 | O |
| ATOM | 354 | CB | LEU | A | 80 | 24.191 | 29.499 | 28.458 | 1.00 | 38.99 | C |
| ATOM | 355 | CG | LEU | A | 80 | 24.665 | 30.846 | 29.001 | 1.00 | 40.69 | C |
| ATOM | 356 | CD1 | LEU | A | 80 | 24.238 | 30.978 | 30.451 | 1.00 | 38.63 | C |
| ATOM | 357 | CD2 | LEU | A | 80 | 26.169 | 30.980 | 28.858 | 1.00 | 32.74 | C |
| ATOM | 358 | N | VAL | A | 81 | 21.515 | 28.861 | 26.837 | 1.00 | 33.48 | N |
| ATOM | 359 | CA | VAL | A | 81 | 20.543 | 29.288 | 25.847 | 1.00 | 38.29 | C |
| ATOM | 360 | C | VAL | A | 81 | 19.122 | 29.205 | 26.404 | 1.00 | 32.64 | C |
| ATOM | 361 | O | VAL | A | 81 | 18.339 | 30.142 | 26.253 | 1.00 | 36.35 | O |
| ATOM | 362 | CB | VAL | A | 81 | 20.716 | 28.530 | 24.510 | 1.00 | 45.83 | C |
| ATOM | 363 | CG1 | VAL | A | 81 | 19.562 | 28.832 | 23.540 | 1.00 | 27.22 | C |
| ATOM | 364 | CG2 | VAL | A | 81 | 22.060 | 28.905 | 23.885 | 1.00 | 29.91 | C |
| ATOM | 365 | N | VAL | A | 82 | 18.799 | 28.098 | 27.065 | 1.00 | 31.63 | N |
| ATOM | 366 | CA | VAL | A | 82 | 17.507 | 27.950 | 27.743 | 1.00 | 34.38 | C |
| ATOM | 367 | C | VAL | A | 82 | 17.228 | 29.104 | 28.703 | 1.00 | 30.41 | C |
| ATOM | 368 | O | VAL | A | 82 | 16.094 | 29.550 | 28.847 | 1.00 | 27.68 | O |
| ATOM | 369 | CB | VAL | A | 82 | 17.442 | 26.644 | 28.546 | 1.00 | 28.52 | C |
| ATOM | 370 | CG1 | VAL | A | 82 | 16.176 | 26.600 | 29.389 | 1.00 | 22.22 | C |
| ATOM | 371 | CG2 | VAL | A | 82 | 17.531 | 25.447 | 27.615 | 1.00 | 26.88 | C |
| ATOM | 372 | N | GLY | A | 83 | 18.281 | 29.586 | 29.350 | 1.00 | 34.08 | N |
| ATOM | 373 | CA | GLY | A | 83 | 18.158 | 30.628 | 30.355 | 1.00 | 39.88 | C |
| ATOM | 374 | C | GLY | A | 83 | 18.156 | 32.061 | 29.851 | 1.00 | 35.32 | C |
| ATOM | 375 | O | GLY | A | 83 | 17.761 | 32.965 | 30.571 | 1.00 | 40.85 | O |
| ATOM | 376 | N | LEU | A | 84 | 18.590 | 32.282 | 28.618 | 1.00 | 37.86 | N |
| ATOM | 377 | CA | LEU | A | 84 | 18.672 | 33.641 | 28.112 | 1.00 | 40.48 | C |
| ATOM | 378 | C | LEU | A | 84 | 17.612 | 33.942 | 27.060 | 1.00 | 39.24 | C |
| ATOM | 379 | O | LEU | A | 84 | 17.124 | 35.067 | 26.971 | 1.00 | 47.74 | O |
| ATOM | 380 | CB | LEU | A | 84 | 20.074 | 33.922 | 27.567 | 1.00 | 40.90 | C |
| ATOM | 381 | CG | LEU | A | 84 | 21.173 | 34.013 | 28.625 | 1.00 | 42.58 | C |
| ATOM | 382 | CD1 | LEU | A | 84 | 22.553 | 34.077 | 27.992 | 1.00 | 32.38 | C |
| ATOM | 383 | CD2 | LEU | A | 84 | 20.926 | 35.226 | 29.487 | 1.00 | 42.05 | C |
| ATOM | 384 | N | LEU | A | 85 | 17.253 | 32.941 | 26.267 | 1.00 | 36.07 | N |
| ATOM | 385 | CA | LEU | A | 85 | 16.299 | 33.153 | 25.177 | 1.00 | 42.26 | C |
| ATOM | 386 | C | LEU | A | 85 | 14.961 | 32.451 | 25.416 | 1.00 | 37.17 | C |
| ATOM | 387 | O | LEU | A | 85 | 13.910 | 33.073 | 25.334 | 1.00 | 43.82 | O |
| ATOM | 388 | CB | LEU | A | 85 | 16.922 | 32.730 | 23.847 | 1.00 | 38.28 | C |
| ATOM | 389 | CG | LEU | A | 85 | 18.295 | 33.386 | 23.659 | 1.00 | 38.79 | C |
| ATOM | 390 | CD1 | LEU | A | 85 | 19.002 | 32.817 | 22.469 | 1.00 | 28.13 | C |
| ATOM | 391 | CD2 | LEU | A | 85 | 18.168 | 34.904 | 23.534 | 1.00 | 36.04 | C |
| ATOM | 392 | N | VAL | A | 86 | 15.008 | 31.166 | 25.745 | 1.00 | 33.87 | N |
| ATOM | 393 | CA | VAL | A | 86 | 13.799 | 30.369 | 25.902 | 1.00 | 27.63 | C |
| ATOM | 394 | C | VAL | A | 86 | 12.941 | 30.804 | 27.104 | 1.00 | 28.87 | C |
| ATOM | 395 | O | VAL | A | 86 | 11.818 | 31.272 | 26.951 | 1.00 | 25.77 | O |
| ATOM | 396 | CB | VAL | A | 86 | 14.147 | 28.864 | 26.035 | 1.00 | 24.34 | C |
| ATOM | 397 | CG1 | VAL | A | 86 | 12.893 | 28.033 | 26.259 | 1.00 | 19.71 | C |
| ATOM | 398 | CG2 | VAL | A | 86 | 14.909 | 28.380 | 24.815 | 1.00 | 19.40 | C |
| ATOM | 399 | N | VAL | A | 87 | 13.476 | 30.638 | 28.305 | 1.00 | 34.72 | N |
| ATOM | 400 | CA | VAL | A | 87 | 12.710 | 30.895 | 29.515 | 1.00 | 29.78 | C |
| ATOM | 401 | C | VAL | A | 87 | 12.222 | 32.348 | 29.652 | 1.00 | 30.68 | C |
| ATOM | 402 | O | VAL | A | 87 | 11.068 | 32.581 | 30.021 | 1.00 | 23.47 | O |
| ATOM | 403 | CB | VAL | A | 87 | 13.485 | 30.439 | 30.781 | 1.00 | 31.77 | C |
| ATOM | 404 | CG1 | VAL | A | 87 | 13.050 | 31.225 | 31.988 | 1.00 | 31.23 | C |
| ATOM | 405 | CG2 | VAL | A | 87 | 13.269 | 28.954 | 31.026 | 1.00 | 32.29 | C |
| ATOM | 406 | N | PRO | A | 88 | 13.095 | 33.330 | 29.369 | 1.00 | 29.30 | N |
| ATOM | 407 | CA | PRO | A | 88 | 12.598 | 34.696 | 29.523 | 1.00 | 28.30 | C |
| ATOM | 408 | C | PRO | A | 88 | 11.377 | 34.970 | 28.659 | 1.00 | 27.72 | C |
| ATOM | 409 | O | PRO | A | 88 | 10.405 | 35.518 | 29.167 | 1.00 | 37.17 | O |
| ATOM | 410 | CB | PRO | A | 88 | 13.787 | 35.554 | 29.107 | 1.00 | 23.80 | C |
| ATOM | 411 | CG | PRO | A | 88 | 14.957 | 34.719 | 29.456 | 1.00 | 27.88 | C |
| ATOM | 412 | CD | PRO | A | 88 | 14.557 | 33.294 | 29.210 | 1.00 | 31.63 | C |
| ATOM | 413 | N | PHE | A | 89 | 11.404 | 34.592 | 27.389 | 1.00 | 33.76 | N |
| ATOM | 414 | CA | PHE | A | 89 | 10.237 | 34.824 | 26.540 | 1.00 | 30.21 | C |
| ATOM | 415 | C | PHE | A | 89 | 9.039 | 33.964 | 26.952 | 1.00 | 30.78 | C |
| ATOM | 416 | O | PHE | A | 89 | 7.904 | 34.434 | 26.950 | 1.00 | 26.22 | O |
| ATOM | 417 | CB | PHE | A | 89 | 10.600 | 34.667 | 25.069 | 1.00 | 24.95 | C |
| ATOM | 418 | CG | PHE | A | 89 | 11.445 | 35.798 | 24.548 | 1.00 | 35.29 | C |
| ATOM | 419 | CD1 | PHE | A | 89 | 10.856 | 36.922 | 23.979 | 1.00 | 30.45 | C |
| ATOM | 420 | CD2 | PHE | A | 89 | 12.829 | 35.759 | 24.664 | 1.00 | 30.00 | C |
| ATOM | 421 | CE1 | PHE | A | 89 | 11.635 | 37.972 | 23.514 | 1.00 | 34.44 | C |
| ATOM | 422 | CE2 | PHE | A | 89 | 13.611 | 36.806 | 24.205 | 1.00 | 25.71 | C |
| ATOM | 423 | CZ | PHE | A | 89 | 13.015 | 37.914 | 23.629 | 1.00 | 28.68 | C |
| ATOM | 424 | N | GLY | A | 90 | 9.304 | 32.718 | 27.336 | 1.00 | 27.60 | N |
| ATOM | 425 | CA | GLY | A | 90 | 8.275 | 31.839 | 27.861 | 1.00 | 28.50 | C |
| ATOM | 426 | C | GLY | A | 90 | 7.575 | 32.405 | 29.088 | 1.00 | 39.02 | C |
| ATOM | 427 | O | GLY | A | 90 | 6.375 | 32.197 | 29.275 | 1.00 | 32.04 | O |
| ATOM | 428 | N | ALA | A | 91 | 8.334 | 33.115 | 29.923 | 1.00 | 38.17 | N |
| ATOM | 429 | CA | ALA | A | 91 | 7.785 | 33.807 | 31.090 | 1.00 | 38.01 | C |
| ATOM | 430 | C | ALA | A | 91 | 6.778 | 34.904 | 30.712 | 1.00 | 41.93 | C |
| ATOM | 431 | O | ALA | A | 91 | 5.712 | 35.003 | 31.324 | 1.00 | 38.41 | O |
| ATOM | 432 | CB | ALA | A | 91 | 8.897 | 34.385 | 31.945 | 1.00 | 23.78 | C |
| ATOM | 433 | N | THR | A | 92 | 7.109 | 35.725 | 29.715 | 1.00 | 30.25 | N |
| ATOM | 434 | CA | THR | A | 92 | 6.186 | 36.772 | 29.268 | 1.00 | 36.62 | C |
| ATOM | 435 | C | THR | A | 92 | 4.868 | 36.163 | 28.810 | 1.00 | 39.14 | C |
| ATOM | 436 | O | THR | A | 92 | 3.800 | 36.710 | 29.069 | 1.00 | 47.30 | O |
| ATOM | 437 | CB | THR | A | 92 | 6.772 | 37.655 | 28.130 | 1.00 | 32.74 | C |
| ATOM | 438 | OG1 | THR | A | 92 | 7.053 | 36.848 | 26.981 | 1.00 | 30.04 | O |
| ATOM | 439 | CG2 | THR | A | 92 | 8.059 | 38.351 | 28.584 | 1.00 | 36.00 | C |
| ATOM | 440 | N | LEU | A | 93 | 4.951 | 35.014 | 28.148 | 1.00 | 36.28 | N |
| ATOM | 441 | CA | LEU | A | 93 | 3.772 | 34.333 | 27.632 | 1.00 | 39.12 | C |
| ATOM | 442 | C | LEU | A | 93 | 2.872 | 33.778 | 28.751 | 1.00 | 44.75 | C |
| ATOM | 443 | O | LEU | A | 93 | 1.691 | 34.115 | 28.847 | 1.00 | 41.41 | O |
| ATOM | 444 | CB | LEU | A | 93 | 4.201 | 33.209 | 26.687 | 1.00 | 35.20 | C |
| ATOM | 445 | CG | LEU | A | 93 | 3.092 | 32.349 | 26.076 | 1.00 | 35.90 | C |
| ATOM | 446 | CD1 | LEU | A | 93 | 2.183 | 33.185 | 25.189 | 1.00 | 33.51 | C |
| ATOM | 447 | CD2 | LEU | A | 93 | 3.696 | 31.221 | 25.285 | 1.00 | 30.48 | C |
| ATOM | 448 | N | VAL | A | 94 | 3.431 | 32.920 | 29.594 | 1.00 | 42.39 | N |
| ATOM | 449 | CA | VAL | A | 94 | 2.649 | 32.328 | 30.665 | 1.00 | 51.25 | C |
| ATOM | 450 | C | VAL | A | 94 | 2.018 | 33.418 | 31.533 | 1.00 | 50.02 | C |
| ATOM | 451 | O | VAL | A | 94 | 0.849 | 33.334 | 31.893 | 1.00 | 48.69 | O |
| ATOM | 452 | CB | VAL | A | 94 | 3.493 | 31.362 | 31.527 | 1.00 | 48.13 | C |
| ATOM | 453 | CG1 | VAL | A | 94 | 2.611 | 30.651 | 32.526 | 1.00 | 49.23 | C |
| ATOM | 454 | CG2 | VAL | A | 94 | 4.189 | 30.340 | 30.646 | 1.00 | 46.44 | C |
| ATOM | 455 | N | VAL | A | 95 | 2.791 | 34.447 | 31.858 | 1.00 | 47.59 | N |
| ATOM | 456 | CA | VAL | A | 95 | 2.266 | 35.553 | 32.651 | 1.00 | 51.50 | C |
| ATOM | 457 | C | VAL | A | 95 | 1.266 | 36.409 | 31.870 | 1.00 | 51.68 | C |
| ATOM | 458 | O | VAL | A | 95 | 0.073 | 36.329 | 32.121 | 1.00 | 58.48 | O |
| ATOM | 459 | CB | VAL | A | 95 | 3.388 | 36.435 | 33.250 | 1.00 | 56.92 | C |
| ATOM | 460 | CG1 | VAL | A | 95 | 2.827 | 37.763 | 33.744 | 1.00 | 56.56 | C |
| ATOM | 461 | CG2 | VAL | A | 95 | 4.095 | 35.701 | 34.387 | 1.00 | 51.83 | C |
| ATOM | 462 | N | ARG | A | 96 | 1.728 | 37.224 | 30.925 | 1.00 | 47.61 | N |
| ATOM | 463 | CA | ARG | A | 96 | 0.808 | 38.124 | 30.228 | 1.00 | 53.03 | C |
| ATOM | 464 | C | ARG | A | 96 | −0.325 | 37.375 | 29.491 | 1.00 | 55.31 | C |
| ATOM | 465 | O | ARG | A | 96 | −1.303 | 37.990 | 29.064 | 1.00 | 52.56 | O |
| ATOM | 466 | CB | ARG | A | 96 | 1.555 | 39.099 | 29.299 | 1.00 | 52.24 | C |
| ATOM | 467 | CG | ARG | A | 96 | 2.577 | 40.009 | 30.008 | 1.00 | 66.10 | C |
| ATOM | 468 | CD | ARG | A | 96 | 2.120 | 41.476 | 30.244 | 1.00 | 77.39 | C |
| ATOM | 469 | NE | ARG | A | 96 | 3.187 | 42.262 | 30.893 | 1.00 | 93.02 | N |
| ATOM | 470 | CZ | ARG | A | 96 | 3.076 | 43.519 | 31.337 | 1.00 | 93.73 | C |
| ATOM | 471 | NH1 | ARG | A | 96 | 1.928 | 44.174 | 31.210 | 1.00 | 91.80 | N |
| ATOM | 472 | NH2 | ARG | A | 96 | 4.119 | 44.125 | 31.916 | 1.00 | 53.01 | N |
| ATOM | 473 | N | GLY | A | 97 | −0.197 | 36.054 | 29.354 | 1.00 | 47.46 | N |
| ATOM | 474 | CA | GLY | A | 97 | −1.247 | 35.245 | 28.760 | 1.00 | 35.30 | C |
| ATOM | 475 | C | GLY | A | 97 | −1.342 | 35.342 | 27.244 | 1.00 | 45.82 | C |
| ATOM | 476 | O | GLY | A | 97 | −2.318 | 34.899 | 26.644 | 1.00 | 44.15 | O |
| ATOM | 477 | N | THR | A | 98 | −0.323 | 35.910 | 26.613 | 1.00 | 49.47 | N |
| ATOM | 478 | CA | THR | A | 98 | −0.352 | 36.123 | 25.165 | 1.00 | 48.86 | C |
| ATOM | 479 | C | THR | A | 98 | 1.056 | 36.232 | 24.593 | 1.00 | 44.15 | C |
| ATOM | 480 | O | THR | A | 98 | 1.990 | 36.624 | 25.306 | 1.00 | 44.96 | O |
| ATOM | 481 | CB | THR | A | 98 | −1.163 | 37.382 | 24.812 | 1.00 | 42.13 | C |
| ATOM | 482 | OG1 | THR | A | 98 | −2.472 | 36.989 | 24.387 | 1.00 | 55.31 | O |
| ATOM | 483 | CG2 | THR | A | 98 | −0.495 | 38.163 | 23.696 | 1.00 | 45.21 | C |
| ATOM | 484 | N | TRP | A | 99 | 1.215 | 35.863 | 23.321 | 1.00 | 32.98 | N |
| ATOM | 485 | CA | TRP | A | 99 | 2.506 | 36.013 | 22.658 | 1.00 | 35.42 | C |
| ATOM | 486 | C | TRP | A | 99 | 2.677 | 37.422 | 22.133 | 1.00 | 30.32 | C |
| ATOM | 487 | O | TRP | A | 99 | 1.857 | 37.895 | 21.365 | 1.00 | 32.08 | O |
| ATOM | 488 | CB | TRP | A | 99 | 2.706 | 35.019 | 21.525 | 1.00 | 29.63 | C |
| ATOM | 489 | CG | TRP | A | 99 | 4.077 | 35.143 | 20.972 | 1.00 | 31.98 | C |
| ATOM | 490 | CD1 | TRP | A | 99 | 4.446 | 35.779 | 19.825 | 1.00 | 31.71 | C |
| ATOM | 491 | CD2 | TRP | A | 99 | 5.284 | 34.663 | 21.571 | 1.00 | 34.38 | C |
| ATOM | 492 | NE1 | TRP | A | 99 | 5.803 | 35.705 | 19.656 | 1.00 | 27.86 | N |
| ATOM | 493 | CE2 | TRP | A | 99 | 6.346 | 35.021 | 20.714 | 1.00 | 39.18 | C |
| ATOM | 494 | CE3 | TRP | A | 99 | 5.571 | 33.956 | 22.743 | 1.00 | 29.09 | C |
| ATOM | 495 | CZ2 | TRP | A | 99 | 7.678 | 34.699 | 20.993 | 1.00 | 30.71 | C |
| ATOM | 496 | CZ3 | TRP | A | 99 | 6.892 | 33.626 | 23.015 | 1.00 | 29.99 | C |
| ATOM | 497 | CH2 | TRP | A | 99 | 7.928 | 34.002 | 22.145 | 1.00 | 26.55 | C |
| ATOM | 498 | N | LEU | A | 100 | 3.760 | 38.077 | 22.553 | 1.00 | 36.74 | N |
| ATOM | 499 | CA | LEU | A | 100 | 3.926 | 39.520 | 22.367 | 1.00 | 33.47 | C |
| ATOM | 500 | C | LEU | A | 100 | 4.969 | 39.874 | 21.324 | 1.00 | 32.65 | C |
| ATOM | 501 | O | LEU | A | 100 | 5.250 | 41.050 | 21.089 | 1.00 | 37.27 | O |
| ATOM | 502 | CB | LEU | A | 100 | 4.318 | 40.181 | 23.695 | 1.00 | 32.20 | C |
| ATOM | 503 | CG | LEU | A | 100 | 3.287 | 40.176 | 24.819 | 1.00 | 46.27 | C |
| ATOM | 504 | CD1 | LEU | A | 100 | 3.875 | 40.752 | 26.100 | 1.00 | 41.60 | C |
| ATOM | 505 | CD2 | LEU | A | 100 | 2.047 | 40.949 | 24.394 | 1.00 | 30.59 | C |
| ATOM | 506 | N | TRP | A | 101 | 5.557 | 38.869 | 20.695 | 1.00 | 29.12 | N |
| ATOM | 507 | CA | TRP | A | 101 | 6.748 | 39.127 | 19.903 | 1.00 | 29.88 | C |
| ATOM | 508 | C | TRP | A | 101 | 6.606 | 38.914 | 18.388 | 1.00 | 34.11 | C |
| ATOM | 509 | O | TRP | A | 101 | 7.568 | 39.096 | 17.647 | 1.00 | 39.62 | O |
| ATOM | 510 | CB | TRP | A | 101 | 7.920 | 38.333 | 20.484 | 1.00 | 31.13 | C |
| ATOM | 511 | CG | TRP | A | 101 | 8.061 | 38.565 | 21.956 | 1.00 | 27.78 | C |
| ATOM | 512 | CD1 | TRP | A | 101 | 7.501 | 37.829 | 22.964 | 1.00 | 31.52 | C |
| ATOM | 513 | CD2 | TRP | A | 101 | 8.788 | 39.616 | 22.590 | 1.00 | 29.18 | C |
| ATOM | 514 | NE1 | TRP | A | 101 | 7.837 | 38.357 | 24.181 | 1.00 | 27.82 | N |
| ATOM | 515 | CE2 | TRP | A | 101 | 8.627 | 39.454 | 23.983 | 1.00 | 25.81 | C |
| ATOM | 516 | CE3 | TRP | A | 101 | 9.571 | 40.668 | 22.116 | 1.00 | 27.74 | C |
| ATOM | 517 | CZ2 | TRP | A | 101 | 9.214 | 40.303 | 24.901 | 1.00 | 25.77 | C |
| ATOM | 518 | CZ3 | TRP | A | 101 | 10.154 | 41.514 | 23.032 | 1.00 | 33.88 | C |
| ATOM | 519 | CH2 | TRP | A | 101 | 9.972 | 41.329 | 24.411 | 1.00 | 38.30 | C |
| ATOM | 520 | N | GLY | A | 102 | 5.417 | 38.549 | 17.921 | 1.00 | 39.83 | N |
| ATOM | 521 | CA | GLY | A | 102 | 5.217 | 38.324 | 16.499 | 1.00 | 41.12 | C |
| ATOM | 522 | C | GLY | A | 102 | 5.430 | 36.870 | 16.133 | 1.00 | 41.74 | C |
| ATOM | 523 | O | GLY | A | 102 | 6.134 | 36.147 | 16.834 | 1.00 | 41.56 | O |
| ATOM | 524 | N | SER | A | 103 | 4.834 | 36.440 | 15.025 | 1.00 | 52.63 | N |
| ATOM | 525 | CA | SER | A | 103 | 4.799 | 35.014 | 14.687 | 1.00 | 52.56 | C |
| ATOM | 526 | C | SER | A | 103 | 6.164 | 34.397 | 14.345 | 1.00 | 41.54 | C |
| ATOM | 527 | O | SER | A | 103 | 6.422 | 33.241 | 14.685 | 1.00 | 44.48 | O |
| ATOM | 528 | CB | SER | A | 103 | 3.772 | 34.731 | 13.589 | 1.00 | 35.57 | C |
| ATOM | 529 | OG | SER | A | 103 | 3.929 | 35.636 | 12.518 | 1.00 | 50.88 | O |
| ATOM | 530 | N | PHE | A | 104 | 7.040 | 35.155 | 13.694 | 1.00 | 33.70 | N |
| ATOM | 531 | CA | PHE | A | 104 | 8.385 | 34.646 | 13.422 | 1.00 | 38.78 | C |
| ATOM | 532 | C | PHE | A | 104 | 9.219 | 34.343 | 14.677 | 1.00 | 39.60 | C |
| ATOM | 533 | O | PHE | A | 104 | 9.855 | 33.282 | 14.781 | 1.00 | 26.66 | O |
| ATOM | 534 | CB | PHE | A | 104 | 9.182 | 35.604 | 12.552 | 1.00 | 35.33 | C |
| ATOM | 535 | CG | PHE | A | 104 | 10.600 | 35.188 | 12.388 | 1.00 | 32.63 | C |
| ATOM | 536 | CD1 | PHE | A | 104 | 10.943 | 34.227 | 11.450 | 1.00 | 32.98 | C |
| ATOM | 537 | CD2 | PHE | A | 104 | 11.589 | 35.714 | 13.205 | 1.00 | 38.27 | C |
| ATOM | 538 | CE1 | PHE | A | 104 | 12.259 | 33.814 | 11.308 | 1.00 | 42.26 | C |
| ATOM | 539 | CE2 | PHE | A | 104 | 12.910 | 35.315 | 13.068 | 1.00 | 38.99 | C |
| ATOM | 540 | CZ | PHE | A | 104 | 13.245 | 34.360 | 12.120 | 1.00 | 36.71 | C |
| ATOM | 541 | N | LEU | A | 105 | 9.248 | 35.291 | 15.609 | 1.00 | 32.02 | N |
| ATOM | 542 | CA | LEU | A | 105 | 9.966 | 35.076 | 16.854 | 1.00 | 32.42 | C |
| ATOM | 543 | C | LEU | A | 105 | 9.352 | 33.914 | 17.634 | 1.00 | 31.91 | C |
| ATOM | 544 | O | LEU | A | 105 | 10.056 | 33.184 | 18.333 | 1.00 | 21.66 | O |
| ATOM | 545 | CB | LEU | A | 105 | 10.017 | 36.356 | 17.681 | 1.00 | 32.02 | C |
| ATOM | 546 | CG | LEU | A | 105 | 11.105 | 37.322 | 17.208 | 1.00 | 32.23 | C |
| ATOM | 547 | CD1 | LEU | A | 105 | 11.122 | 38.608 | 18.042 | 1.00 | 27.17 | C |
| ATOM | 548 | CD2 | LEU | A | 105 | 12.455 | 36.630 | 17.228 | 1.00 | 25.20 | C |
| ATOM | 549 | N | CYS | A | 106 | 8.043 | 33.731 | 17.489 | 1.00 | 28.81 | N |
| ATOM | 550 | CA | CYS | A | 106 | 7.383 | 32.573 | 18.069 | 1.00 | 24.29 | C |
| ATOM | 551 | C | CYS | A | 106 | 7.949 | 31.277 | 17.484 | 1.00 | 28.87 | C |
| ATOM | 552 | O | CYS | A | 106 | 8.253 | 30.329 | 18.211 | 1.00 | 23.80 | O |
| ATOM | 553 | CB | CYS | A | 106 | 5.874 | 32.642 | 17.851 | 1.00 | 29.79 | C |
| ATOM | 554 | SG | CYS | A | 106 | 5.004 | 31.087 | 18.158 | 1.00 | 29.48 | S |
| ATOM | 555 | N | GLU | A | 107 | 8.113 | 31.235 | 16.168 | 1.00 | 29.13 | N |
| ATOM | 556 | CA | GLU | A | 107 | 8.623 | 30.024 | 15.535 | 1.00 | 25.62 | C |
| ATOM | 557 | C | GLU | A | 107 | 10.128 | 29.835 | 15.734 | 1.00 | 25.27 | C |
| ATOM | 558 | O | GLU | A | 107 | 10.605 | 28.706 | 15.860 | 1.00 | 20.16 | O |
| ATOM | 559 | CB | GLU | A | 107 | 8.231 | 29.975 | 14.064 | 1.00 | 27.94 | C |
| ATOM | 560 | CG | GLU | A | 107 | 6.746 | 29.796 | 13.873 | 1.00 | 26.15 | C |
| ATOM | 561 | CD | GLU | A | 107 | 6.289 | 30.081 | 12.462 | 1.00 | 37.99 | C |
| ATOM | 562 | OE1 | GLU | A | 107 | 7.015 | 29.720 | 11.518 | 1.00 | 49.31 | O |
| ATOM | 563 | OE2 | GLU | A | 107 | 5.192 | 30.660 | 12.292 | 1.00 | 52.40 | O |
| ATOM | 564 | N | LEU | A | 108 | 10.872 | 30.937 | 15.790 | 1.00 | 21.61 | N |
| ATOM | 565 | CA | LEU | A | 108 | 12.284 | 30.857 | 16.160 | 1.00 | 27.69 | C |
| ATOM | 566 | C | LEU | A | 108 | 12.479 | 30.375 | 17.613 | 1.00 | 31.99 | C |
| ATOM | 567 | O | LEU | A | 108 | 13.351 | 29.542 | 17.897 | 1.00 | 25.09 | O |
| ATOM | 568 | CB | LEU | A | 108 | 12.975 | 32.200 | 15.940 | 1.00 | 25.86 | C |
| ATOM | 569 | CG | LEU | A | 108 | 14.450 | 32.182 | 16.347 | 1.00 | 25.53 | C |
| ATOM | 570 | CD1 | LEU | A | 108 | 15.156 | 30.996 | 15.695 | 1.00 | 22.16 | C |
| ATOM | 571 | CD2 | LEU | A | 108 | 15.140 | 33.512 | 16.017 | 1.00 | 18.26 | C |
| ATOM | 572 | N | TRP | A | 109 | 11.649 | 30.913 | 18.510 | 1.00 | 26.41 | N |
| ATOM | 573 | CA | TRP | A | 109 | 11.617 | 30.561 | 19.930 | 1.00 | 23.93 | C |
| ATOM | 574 | C | TRP | A | 109 | 11.379 | 29.072 | 20.166 | 1.00 | 32.19 | C |
| ATOM | 575 | O | TRP | A | 109 | 12.103 | 28.426 | 20.932 | 1.00 | 29.43 | O |
| ATOM | 576 | CB | TRP | A | 109 | 10.504 | 31.349 | 20.621 | 1.00 | 21.63 | C |
| ATOM | 577 | CG | TRP | A | 109 | 10.321 | 31.016 | 22.063 | 1.00 | 25.45 | C |
| ATOM | 578 | CD1 | TRP | A | 109 | 11.231 | 31.196 | 23.065 | 1.00 | 22.97 | C |
| ATOM | 579 | CD2 | TRP | A | 109 | 9.146 | 30.470 | 22.682 | 1.00 | 32.06 | C |
| ATOM | 580 | NE1 | TRP | A | 109 | 10.707 | 30.777 | 24.263 | 1.00 | 25.31 | N |
| ATOM | 581 | CE2 | TRP | A | 109 | 9.429 | 30.328 | 24.062 | 1.00 | 27.98 | C |
| ATOM | 582 | CE3 | TRP | A | 109 | 7.885 | 30.077 | 22.205 | 1.00 | 26.29 | C |
| ATOM | 583 | CZ2 | TRP | A | 109 | 8.499 | 29.811 | 24.972 | 1.00 | 27.53 | C |
| ATOM | 584 | CZ3 | TRP | A | 109 | 6.961 | 29.561 | 23.114 | 1.00 | 31.36 | C |
| ATOM | 585 | CH2 | TRP | A | 109 | 7.274 | 29.439 | 24.483 | 1.00 | 27.80 | C |
| ATOM | 586 | N | THR | A | 110 | 10.340 | 28.551 | 19.518 | 1.00 | 23.31 | N |
| ATOM | 587 | CA | THR | A | 110 | 9.979 | 27.150 | 19.608 | 1.00 | 23.07 | C |
| ATOM | 588 | C | THR | A | 110 | 11.140 | 26.285 | 19.146 | 1.00 | 30.79 | C |
| ATOM | 589 | O | THR | A | 110 | 11.398 | 25.196 | 19.686 | 1.00 | 18.74 | O |
| ATOM | 590 | CB | THR | A | 110 | 8.766 | 26.864 | 18.708 | 1.00 | 27.49 | C |
| ATOM | 591 | OG1 | THR | A | 110 | 7.652 | 27.626 | 19.174 | 1.00 | 27.85 | O |
| ATOM | 592 | CG2 | THR | A | 110 | 8.396 | 25.372 | 18.705 | 1.00 | 16.96 | C |
| ATOM | 593 | N | SER | A | 111 | 11.827 | 26.787 | 18.125 | 1.00 | 30.93 | N |
| ATOM | 594 | CA | SER | A | 111 | 13.000 | 26.134 | 17.564 | 1.00 | 29.34 | C |
| ATOM | 595 | C | SER | A | 111 | 14.124 | 25.964 | 18.584 | 1.00 | 32.82 | C |
| ATOM | 596 | O | SER | A | 111 | 14.658 | 24.864 | 18.745 | 1.00 | 29.79 | O |
| ATOM | 597 | CB | SER | A | 111 | 13.527 | 26.950 | 16.387 | 1.00 | 30.22 | C |
| ATOM | 598 | OG | SER | A | 111 | 12.801 | 26.664 | 15.207 | 1.00 | 49.18 | O |
| ATOM | 599 | N | LEU | A | 112 | 14.488 | 27.061 | 19.250 | 1.00 | 25.83 | N |
| ATOM | 600 | CA | LEU | A | 112 | 15.567 | 27.046 | 20.227 | 1.00 | 22.76 | C |
| ATOM | 601 | C | LEU | A | 112 | 15.179 | 26.150 | 21.395 | 1.00 | 24.47 | C |
| ATOM | 602 | O | LEU | A | 112 | 15.998 | 25.405 | 21.939 | 1.00 | 20.91 | O |
| ATOM | 603 | CB | LEU | A | 112 | 15.880 | 28.470 | 20.692 | 1.00 | 17.00 | C |
| ATOM | 604 | CG | LEU | A | 112 | 16.298 | 29.405 | 19.552 | 1.00 | 25.17 | C |
| ATOM | 605 | CD1 | LEU | A | 112 | 16.361 | 30.866 | 19.971 | 1.00 | 18.57 | C |
| ATOM | 606 | CD2 | LEU | A | 112 | 17.628 | 28.963 | 18.962 | 1.00 | 18.70 | C |
| ATOM | 607 | N | ASP | A | 113 | 13.909 | 26.205 | 21.762 | 1.00 | 17.82 | N |
| ATOM | 608 | CA | ASP | A | 113 | 13.410 | 25.374 | 22.837 | 1.00 | 20.69 | C |
| ATOM | 609 | C | ASP | A | 113 | 13.584 | 23.889 | 22.484 | 1.00 | 25.82 | C |
| ATOM | 610 | O | ASP | A | 113 | 14.059 | 23.104 | 23.302 | 1.00 | 27.43 | O |
| ATOM | 611 | CB | ASP | A | 113 | 11.951 | 25.724 | 23.101 | 1.00 | 20.34 | C |
| ATOM | 612 | CG | ASP | A | 113 | 11.414 | 25.091 | 24.344 | 1.00 | 20.22 | C |
| ATOM | 613 | OD1 | ASP | A | 113 | 11.950 | 24.070 | 24.800 | 1.00 | 25.57 | O |
| ATOM | 614 | OD2 | ASP | A | 113 | 10.435 | 25.621 | 24.874 | 1.00 | 28.51 | O |
| ATOM | 615 | N | VAL | A | 114 | 13.226 | 23.521 | 21.256 | 1.00 | 22.54 | N |
| ATOM | 616 | CA | VAL | A | 114 | 13.399 | 22.151 | 20.771 | 1.00 | 23.64 | C |
| ATOM | 617 | C | VAL | A | 114 | 14.872 | 21.729 | 20.637 | 1.00 | 25.12 | C |
| ATOM | 618 | O | VAL | A | 114 | 15.255 | 20.621 | 21.044 | 1.00 | 24.22 | O |
| ATOM | 619 | CB | VAL | A | 114 | 12.679 | 21.952 | 19.425 | 1.00 | 21.36 | C |
| ATOM | 620 | CG1 | VAL | A | 114 | 12.823 | 20.526 | 18.949 | 1.00 | 21.22 | C |
| ATOM | 621 | CG2 | VAL | A | 114 | 11.223 | 22.292 | 19.577 | 1.00 | 25.23 | C |
| ATOM | 622 | N | LEU | A | 115 | 15.684 | 22.616 | 20.068 | 1.00 | 18.85 | N |
| ATOM | 623 | CA | LEU | A | 115 | 17.131 | 22.430 | 19.966 | 1.00 | 17.94 | C |
| ATOM | 624 | C | LEU | A | 115 | 17.785 | 22.008 | 21.272 | 1.00 | 30.95 | C |
| ATOM | 625 | O | LEU | A | 115 | 18.519 | 21.007 | 21.323 | 1.00 | 23.36 | O |
| ATOM | 626 | CB | LEU | A | 115 | 17.780 | 23.735 | 19.515 | 1.00 | 17.88 | C |
| ATOM | 627 | CG | LEU | A | 115 | 19.278 | 23.694 | 19.229 | 1.00 | 28.01 | C |
| ATOM | 628 | CD1 | LEU | A | 115 | 19.590 | 22.744 | 18.083 | 1.00 | 20.89 | C |
| ATOM | 629 | CD2 | LEU | A | 115 | 19.779 | 25.092 | 18.928 | 1.00 | 23.88 | C |
| ATOM | 630 | N | CYS | A | 116 | 17.512 | 22.784 | 22.325 | 1.00 | 32.44 | N |
| ATOM | 631 | CA | CYS | A | 116 | 18.230 | 22.675 | 23.586 | 1.00 | 20.80 | C |
| ATOM | 632 | C | CYS | A | 116 | 17.958 | 21.361 | 24.305 | 1.00 | 23.94 | C |
| ATOM | 633 | O | CYS | A | 116 | 18.884 | 20.718 | 24.797 | 1.00 | 26.96 | O |
| ATOM | 634 | CB | CYS | A | 116 | 17.916 | 23.872 | 24.475 | 1.00 | 25.38 | C |
| ATOM | 635 | SG | CYS | A | 116 | 18.713 | 25.395 | 23.919 | 1.00 | 33.66 | S |
| ATOM | 636 | N | VAL | A | 117 | 16.698 | 20.948 | 24.355 | 1.00 | 17.87 | N |
| ATOM | 637 | CA | VAL | A | 117 | 16.364 | 19.634 | 24.900 | 1.00 | 18.68 | C |
| ATOM | 638 | C | VAL | A | 117 | 17.058 | 18.527 | 24.103 | 1.00 | 21.45 | C |
| ATOM | 639 | O | VAL | A | 117 | 17.612 | 17.592 | 24.669 | 1.00 | 16.69 | O |
| ATOM | 640 | CB | VAL | A | 117 | 14.838 | 19.376 | 24.867 | 1.00 | 19.25 | C |
| ATOM | 641 | CG1 | VAL | A | 117 | 14.498 | 18.031 | 25.492 | 1.00 | 15.98 | C |
| ATOM | 642 | CG2 | VAL | A | 117 | 14.108 | 20.484 | 25.560 | 1.00 | 21.84 | C |
| ATOM | 643 | N | THR | A | 118 | 17.021 | 18.641 | 22.778 | 1.00 | 24.64 | N |
| ATOM | 644 | CA | THR | A | 118 | 17.640 | 17.655 | 21.907 | 1.00 | 23.06 | C |
| ATOM | 645 | C | THR | A | 118 | 19.159 | 17.585 | 22.086 | 1.00 | 22.58 | C |
| ATOM | 646 | O | THR | A | 118 | 19.723 | 16.500 | 22.249 | 1.00 | 19.86 | O |
| ATOM | 647 | CB | THR | A | 118 | 17.290 | 17.943 | 20.446 | 1.00 | 25.18 | C |
| ATOM | 648 | OG1 | THR | A | 118 | 15.862 | 17.976 | 20.316 | 1.00 | 31.14 | O |
| ATOM | 649 | CG2 | THR | A | 118 | 17.865 | 16.872 | 19.531 | 1.00 | 21.94 | C |
| ATOM | 650 | N | ALA | A | 119 | 19.818 | 18.738 | 22.074 | 1.00 | 13.86 | N |
| ATOM | 651 | CA | ALA | A | 119 | 21.259 | 18.755 | 22.259 | 1.00 | 16.35 | C |
| ATOM | 652 | C | ALA | A | 119 | 21.682 | 18.219 | 23.644 | 1.00 | 25.85 | C |
| ATOM | 653 | O | ALA | A | 119 | 22.705 | 17.546 | 23.765 | 1.00 | 23.32 | O |
| ATOM | 654 | CB | ALA | A | 119 | 21.805 | 20.155 | 22.031 | 1.00 | 17.40 | C |
| ATOM | 655 | N | SER | A | 120 | 20.906 | 18.527 | 24.685 | 1.00 | 19.69 | N |
| ATOM | 656 | CA | SER | A | 120 | 21.248 | 18.111 | 26.039 | 1.00 | 17.57 | C |
| ATOM | 657 | C | SER | A | 120 | 21.300 | 16.601 | 26.146 | 1.00 | 22.49 | C |
| ATOM | 658 | O | SER | A | 120 | 22.303 | 16.030 | 26.580 | 1.00 | 22.20 | O |
| ATOM | 659 | CB | SER | A | 120 | 20.245 | 18.651 | 27.051 | 1.00 | 17.38 | C |
| ATOM | 660 | OG | SER | A | 120 | 20.324 | 20.062 | 27.147 | 1.00 | 26.82 | O |
| ATOM | 661 | N | ILE | A | 121 | 20.210 | 15.960 | 25.735 | 1.00 | 23.45 | N |
| ATOM | 662 | CA | ILE | A | 121 | 20.060 | 14.523 | 25.876 | 1.00 | 21.41 | C |
| ATOM | 663 | C | ILE | A | 121 | 21.031 | 13.795 | 24.948 | 1.00 | 21.50 | C |
| ATOM | 664 | O | ILE | A | 121 | 21.508 | 12.709 | 25.260 | 1.00 | 29.42 | O |
| ATOM | 665 | CB | ILE | A | 121 | 18.611 | 14.095 | 25.624 | 1.00 | 20.38 | C |
| ATOM | 666 | CG1 | ILE | A | 121 | 18.468 | 12.579 | 25.712 | 1.00 | 23.98 | C |
| ATOM | 667 | CG2 | ILE | A | 121 | 18.152 | 14.596 | 24.270 | 1.00 | 25.87 | C |
| ATOM | 668 | CD1 | ILE | A | 121 | 19.035 | 11.994 | 26.963 | 1.00 | 19.99 | C |
| ATOM | 669 | N | GLU | A | 122 | 21.352 | 14.391 | 23.813 | 1.00 | 19.49 | N |
| ATOM | 670 | CA | GLU | A | 122 | 22.384 | 13.796 | 22.977 | 1.00 | 25.68 | C |
| ATOM | 671 | C | GLU | A | 122 | 23.759 | 13.899 | 23.666 | 1.00 | 26.31 | C |
| ATOM | 672 | O | GLU | A | 122 | 24.495 | 12.916 | 23.768 | 1.00 | 23.49 | O |
| ATOM | 673 | CB | GLU | A | 122 | 22.380 | 14.414 | 21.573 | 1.00 | 24.16 | C |
| ATOM | 674 | CG | GLU | A | 122 | 21.217 | 13.928 | 20.708 | 1.00 | 36.65 | C |
| ATOM | 675 | CD | GLU | A | 122 | 21.259 | 14.435 | 19.266 | 1.00 | 51.16 | C |
| ATOM | 676 | OE1 | GLU | A | 122 | 22.372 | 14.562 | 18.699 | 1.00 | 53.58 | O |
| ATOM | 677 | OE2 | GLU | A | 122 | 20.169 | 14.684 | 18.693 | 1.00 | 43.13 | O |
| ATOM | 678 | N | THR | A | 123 | 24.082 | 15.085 | 24.167 | 1.00 | 20.57 | N |
| ATOM | 679 | CA | THR | A | 123 | 25.325 | 15.289 | 24.890 | 1.00 | 20.18 | C |
| ATOM | 680 | C | THR | A | 123 | 25.466 | 14.333 | 26.084 | 1.00 | 26.78 | C |
| ATOM | 681 | O | THR | A | 123 | 26.564 | 13.863 | 26.389 | 1.00 | 24.79 | O |
| ATOM | 682 | CB | THR | A | 123 | 25.486 | 16.753 | 25.346 | 1.00 | 24.64 | C |
| ATOM | 683 | OG1 | THR | A | 123 | 25.525 | 17.621 | 24.202 | 1.00 | 26.06 | O |
| ATOM | 684 | CG2 | THR | A | 123 | 26.769 | 16.930 | 26.136 | 1.00 | 26.04 | C |
| ATOM | 685 | N | LEU | A | 124 | 24.356 | 14.034 | 26.753 | 1.00 | 24.32 | N |
| ATOM | 686 | CA | LEU | A | 124 | 24.380 | 13.087 | 27.865 | 1.00 | 19.80 | C |
| ATOM | 687 | C | LEU | A | 124 | 24.688 | 11.676 | 27.383 | 1.00 | 28.83 | C |
| ATOM | 688 | O | LEU | A | 124 | 25.385 | 10.930 | 28.066 | 1.00 | 27.68 | O |
| ATOM | 689 | CB | LEU | A | 124 | 23.056 | 13.092 | 28.633 | 1.00 | 22.50 | C |
| ATOM | 690 | CG | LEU | A | 124 | 22.785 | 14.283 | 29.554 | 1.00 | 25.11 | C |
| ATOM | 691 | CD1 | LEU | A | 124 | 21.442 | 14.145 | 30.238 | 1.00 | 24.23 | C |
| ATOM | 692 | CD2 | LEU | A | 124 | 23.878 | 14.441 | 30.574 | 1.00 | 18.69 | C |
| ATOM | 693 | N | CYS | A | 125 | 24.158 | 11.305 | 26.220 | 1.00 | 26.56 | N |
| ATOM | 694 | CA | CYS | A | 125 | 24.519 | 10.046 | 25.580 | 1.00 | 30.82 | C |
| ATOM | 695 | C | CYS | A | 125 | 26.018 | 9.922 | 25.363 | 1.00 | 31.05 | C |
| ATOM | 696 | O | CYS | A | 125 | 26.629 | 8.917 | 25.731 | 1.00 | 28.69 | O |
| ATOM | 697 | CB | CYS | A | 125 | 23.847 | 9.935 | 24.221 | 1.00 | 31.32 | C |
| ATOM | 698 | SG | CYS | A | 125 | 22.263 | 9.213 | 24.305 | 1.00 | 49.40 | S |
| ATOM | 699 | N | VAL | A | 126 | 26.593 | 10.944 | 24.734 | 1.00 | 25.97 | N |
| ATOM | 700 | CA | VAL | A | 126 | 28.018 | 10.962 | 24.434 | 1.00 | 30.63 | C |
| ATOM | 701 | C | VAL | A | 126 | 28.825 | 10.813 | 25.719 | 1.00 | 32.81 | C |
| ATOM | 702 | O | VAL | A | 126 | 29.790 | 10.058 | 25.762 | 1.00 | 35.10 | O |
| ATOM | 703 | CB | VAL | A | 126 | 28.428 | 12.267 | 23.720 | 1.00 | 34.42 | C |
| ATOM | 704 | CG1 | VAL | A | 126 | 29.943 | 12.426 | 23.715 | 1.00 | 26.18 | C |
| ATOM | 705 | CG2 | VAL | A | 126 | 27.851 | 12.316 | 22.303 | 1.00 | 22.68 | C |
| ATOM | 706 | N | ILE | A | 127 | 28.419 | 11.529 | 26.765 | 1.00 | 27.77 | N |
| ATOM | 707 | CA | ILE | A | 127 | 29.070 | 11.407 | 28.061 | 1.00 | 29.29 | C |
| ATOM | 708 | C | ILE | A | 127 | 29.058 | 9.966 | 28.612 | 1.00 | 31.65 | C |
| ATOM | 709 | O | ILE | A | 127 | 30.076 | 9.478 | 29.093 | 1.00 | 35.89 | O |
| ATOM | 710 | CB | ILE | A | 127 | 28.484 | 12.399 | 29.081 | 1.00 | 29.77 | C |
| ATOM | 711 | CG1 | ILE | A | 127 | 28.858 | 13.832 | 28.695 | 1.00 | 21.67 | C |
| ATOM | 712 | CG2 | ILE | A | 127 | 28.990 | 12.087 | 30.474 | 1.00 | 23.05 | C |
| ATOM | 713 | CD1 | ILE | A | 127 | 28.046 | 14.880 | 29.383 | 1.00 | 19.91 | C |
| ATOM | 714 | N | ALA | A | 128 | 27.922 | 9.282 | 28.525 | 1.00 | 27.16 | N |
| ATOM | 715 | CA | ALA | A | 128 | 27.853 | 7.877 | 28.927 | 1.00 | 31.35 | C |
| ATOM | 716 | C | ALA | A | 128 | 28.800 | 6.995 | 28.109 | 1.00 | 34.42 | C |
| ATOM | 717 | O | ALA | A | 128 | 29.569 | 6.218 | 28.661 | 1.00 | 42.98 | O |
| ATOM | 718 | CB | ALA | A | 128 | 26.424 | 7.354 | 28.821 | 1.00 | 32.42 | C |
| ATOM | 719 | N | ILE | A | 129 | 28.724 | 7.110 | 26.794 | 1.00 | 25.63 | N |
| ATOM | 720 | CA | ILE | A | 129 | 29.538 | 6.302 | 25.893 | 1.00 | 32.80 | C |
| ATOM | 721 | C | ILE | A | 129 | 31.041 | 6.551 | 26.100 | 1.00 | 39.23 | C |
| ATOM | 722 | O | ILE | A | 129 | 31.850 | 5.622 | 26.050 | 1.00 | 35.36 | O |
| ATOM | 723 | CB | ILE | A | 129 | 29.122 | 6.552 | 24.408 | 1.00 | 31.99 | C |
| ATOM | 724 | CG1 | ILE | A | 129 | 27.817 | 5.821 | 24.100 | 1.00 | 25.34 | C |
| ATOM | 725 | CG2 | ILE | A | 129 | 30.215 | 6.134 | 23.431 | 1.00 | 18.91 | C |
| ATOM | 726 | CD1 | ILE | A | 129 | 27.126 | 6.327 | 22.883 | 1.00 | 16.13 | C |
| ATOM | 727 | N | ASP | A | 130 | 31.395 | 7.810 | 26.343 | 1.00 | 36.21 | N |
| ATOM | 728 | CA | ASP | A | 130 | 32.777 | 8.232 | 26.540 | 1.00 | 32.64 | C |
| ATOM | 729 | C | ASP | A | 130 | 33.348 | 7.576 | 27.792 | 1.00 | 39.60 | C |
| ATOM | 730 | O | ASP | A | 130 | 34.435 | 6.995 | 27.757 | 1.00 | 39.45 | O |
| ATOM | 731 | CB | ASP | A | 130 | 32.822 | 9.770 | 26.638 | 1.00 | 44.57 | C |
| ATOM | 732 | CG | ASP | A | 130 | 34.147 | 10.311 | 27.188 | 1.00 | 57.37 | C |
| ATOM | 733 | OD1 | ASP | A | 130 | 35.171 | 10.292 | 26.462 | 1.00 | 55.76 | O |
| ATOM | 734 | OD2 | ASP | A | 130 | 34.149 | 10.804 | 28.342 | 1.00 | 45.48 | O |
| ATOM | 735 | N | ARG | A | 131 | 32.598 | 7.660 | 28.891 | 1.00 | 40.43 | N |
| ATOM | 736 | CA | ARG | A | 131 | 32.989 | 7.030 | 30.152 | 1.00 | 40.13 | C |
| ATOM | 737 | C | ARG | A | 131 | 33.063 | 5.504 | 30.036 | 1.00 | 34.50 | C |
| ATOM | 738 | O | ARG | A | 131 | 34.053 | 4.896 | 30.425 | 1.00 | 44.83 | O |
| ATOM | 739 | CB | ARG | A | 131 | 32.048 | 7.450 | 31.289 | 1.00 | 29.24 | C |
| ATOM | 740 | CG | ARG | A | 131 | 32.278 | 8.875 | 31.807 | 1.00 | 34.21 | C |
| ATOM | 741 | CD | ARG | A | 131 | 33.714 | 9.113 | 32.252 | 1.00 | 37.61 | C |
| ATOM | 742 | NE | ARG | A | 131 | 34.599 | 9.430 | 31.137 | 1.00 | 43.94 | N |
| ATOM | 743 | CZ | ARG | A | 131 | 35.926 | 9.381 | 31.194 | 1.00 | 48.00 | C |
| ATOM | 744 | NH1 | ARG | A | 131 | 36.532 | 9.017 | 32.314 | 1.00 | 59.85 | N |
| ATOM | 745 | NH2 | ARG | A | 131 | 36.650 | 9.691 | 30.129 | 1.00 | 49.97 | N |
| ATOM | 746 | N | TYR | A | 132 | 32.018 | 4.887 | 29.504 | 1.00 | 28.51 | N |
| ATOM | 747 | CA | TYR | A | 132 | 32.041 | 3.454 | 29.255 | 1.00 | 41.61 | C |
| ATOM | 748 | C | TYR | A | 132 | 33.282 | 2.996 | 28.485 | 1.00 | 45.44 | C |
| ATOM | 749 | O | TYR | A | 132 | 33.866 | 1.958 | 28.792 | 1.00 | 43.33 | O |
| ATOM | 750 | CB | TYR | A | 132 | 30.790 | 3.021 | 28.494 | 1.00 | 40.24 | C |
| ATOM | 751 | CG | TYR | A | 132 | 30.865 | 1.588 | 28.040 | 1.00 | 50.36 | C |
| ATOM | 752 | CD1 | TYR | A | 132 | 30.509 | 0.552 | 28.894 | 1.00 | 50.22 | C |
| ATOM | 753 | CD2 | TYR | A | 132 | 31.318 | 1.267 | 26.765 | 1.00 | 50.69 | C |
| ATOM | 754 | CE1 | TYR | A | 132 | 30.584 | −0.761 | 28.490 | 1.00 | 48.44 | C |
| ATOM | 755 | CE2 | TYR | A | 132 | 31.394 | −0.047 | 26.349 | 1.00 | 49.88 | C |
| ATOM | 756 | CZ | TYR | A | 132 | 31.025 | −1.054 | 27.216 | 1.00 | 55.07 | C |
| ATOM | 757 | OH | TYR | A | 132 | 31.104 | −2.363 | 26.815 | 1.00 | 71.99 | O |
| ATOM | 758 | N | LEU | A | 133 | 33.673 | 3.763 | 27.474 | 1.00 | 40.17 | N |
| ATOM | 759 | CA | LEU | A | 133 | 34.835 | 3.408 | 26.663 | 1.00 | 43.76 | C |
| ATOM | 760 | C | LEU | A | 133 | 36.148 | 3.710 | 27.372 | 1.00 | 52.16 | C |
| ATOM | 761 | O | LEU | A | 133 | 37.132 | 3.002 | 27.181 | 1.00 | 57.70 | O |
| ATOM | 762 | CB | LEU | A | 133 | 34.799 | 4.096 | 25.295 | 1.00 | 42.37 | C |
| ATOM | 763 | CG | LEU | A | 133 | 33.717 | 3.594 | 24.329 | 1.00 | 49.80 | C |
| ATOM | 764 | CD1 | LEU | A | 133 | 33.781 | 4.345 | 23.016 | 1.00 | 24.35 | C |
| ATOM | 765 | CD2 | LEU | A | 133 | 33.801 | 2.086 | 24.088 | 1.00 | 43.34 | C |
| ATOM | 766 | N | ALA | A | 134 | 36.164 | 4.753 | 28.193 | 1.00 | 44.11 | N |
| ATOM | 767 | CA | ALA | A | 134 | 37.358 | 5.071 | 28.958 | 1.00 | 41.10 | C |
| ATOM | 768 | C | ALA | A | 134 | 37.628 | 4.007 | 30.021 | 1.00 | 52.54 | C |
| ATOM | 769 | O | ALA | A | 134 | 38.697 | 3.985 | 30.635 | 1.00 | 60.51 | O |
| ATOM | 770 | CB | ALA | A | 134 | 37.236 | 6.439 | 29.595 | 1.00 | 32.00 | C |
| ATOM | 771 | N | ILE | A | 135 | 36.667 | 3.108 | 30.218 | 1.00 | 50.87 | N |
| ATOM | 772 | CA | ILE | A | 135 | 36.682 | 2.230 | 31.384 | 1.00 | 57.17 | C |
| ATOM | 773 | C | ILE | A | 135 | 36.814 | 0.749 | 31.050 | 1.00 | 50.48 | C |
| ATOM | 774 | O | ILE | A | 135 | 36.780 | −0.092 | 31.937 | 1.00 | 64.50 | O |
| ATOM | 775 | CB | ILE | A | 135 | 35.408 | 2.425 | 32.229 | 1.00 | 49.89 | C |
| ATOM | 776 | CG1 | ILE | A | 135 | 35.746 | 2.550 | 33.710 | 1.00 | 53.84 | C |
| ATOM | 777 | CG2 | ILE | A | 135 | 34.430 | 1.291 | 32.013 | 1.00 | 51.13 | C |
| ATOM | 778 | CD1 | ILE | A | 135 | 34.508 | 2.637 | 34.585 | 1.00 | 61.01 | C |
| ATOM | 779 | N | THR | A | 136 | 36.961 | 0.429 | 29.775 | 1.00 | 56.71 | N |
| ATOM | 780 | CA | THR | A | 136 | 37.014 | −0.965 | 29.349 | 1.00 | 61.77 | C |
| ATOM | 781 | C | THR | A | 136 | 38.064 | −1.155 | 28.257 | 1.00 | 68.81 | C |
| ATOM | 782 | O | THR | A | 136 | 38.435 | −2.278 | 27.911 | 1.00 | 68.01 | O |
| ATOM | 783 | CB | THR | A | 136 | 35.634 | −1.452 | 28.828 | 1.00 | 62.73 | C |
| ATOM | 784 | OG1 | THR | A | 136 | 35.207 | −0.633 | 27.735 | 1.00 | 53.79 | O |
| ATOM | 785 | CG2 | THR | A | 136 | 34.587 | −1.389 | 29.926 | 1.00 | 57.04 | C |
| ATOM | 786 | N | SER | A | 137 | 38.542 | −0.037 | 27.724 | 1.00 | 59.43 | N |
| ATOM | 787 | CA | SER | A | 137 | 39.503 | −0.042 | 26.639 | 1.00 | 61.67 | C |
| ATOM | 788 | C | SER | A | 137 | 40.381 | 1.188 | 26.793 | 1.00 | 54.76 | C |
| ATOM | 789 | O | SER | A | 137 | 40.530 | 1.965 | 25.849 | 1.00 | 51.16 | O |
| ATOM | 790 | CB | SER | A | 137 | 38.763 | 0.009 | 25.299 | 1.00 | 57.88 | C |
| ATOM | 791 | OG | SER | A | 137 | 37.514 | −0.661 | 25.392 | 1.00 | 52.55 | O |
| ATOM | 792 | N | PRO | A | 138 | 40.971 | 1.362 | 27.989 | 1.00 | 50.19 | N |
| ATOM | 793 | CA | PRO | A | 138 | 41.644 | 2.604 | 28.394 | 1.00 | 51.45 | C |
| ATOM | 794 | C | PRO | A | 138 | 42.815 | 2.962 | 27.493 | 1.00 | 55.73 | C |
| ATOM | 795 | O | PRO | A | 138 | 43.122 | 4.145 | 27.331 | 1.00 | 53.65 | O |
| ATOM | 796 | CB | PRO | A | 138 | 42.152 | 2.294 | 29.809 | 1.00 | 45.34 | C |
| ATOM | 797 | CG | PRO | A | 138 | 41.438 | 1.061 | 30.236 | 1.00 | 47.72 | C |
| ATOM | 798 | CD | PRO | A | 138 | 41.147 | 0.295 | 28.984 | 1.00 | 50.12 | C |
| ATOM | 799 | N | PHE | A | 139 | 43.472 | 1.959 | 26.919 | 1.00 | 58.57 | N |
| ATOM | 800 | CA | PHE | A | 139 | 44.595 | 2.247 | 26.041 | 1.00 | 58.56 | C |
| ATOM | 801 | C | PHE | A | 139 | 44.075 | 2.902 | 24.790 | 1.00 | 55.67 | C |
| ATOM | 802 | O | PHE | A | 139 | 44.411 | 4.048 | 24.486 | 1.00 | 52.96 | O |
| ATOM | 803 | CB | PHE | A | 139 | 45.364 | 0.990 | 25.660 | 1.00 | 59.59 | C |
| ATOM | 804 | CG | PHE | A | 139 | 46.527 | 1.262 | 24.754 | 1.00 | 60.38 | C |
| ATOM | 805 | CD1 | PHE | A | 139 | 47.688 | 1.832 | 25.251 | 1.00 | 59.05 | C |
| ATOM | 806 | CD2 | PHE | A | 139 | 46.454 | 0.969 | 23.401 | 1.00 | 64.77 | C |
| ATOM | 807 | CE1 | PHE | A | 139 | 48.759 | 2.091 | 24.420 | 1.00 | 63.99 | C |
| ATOM | 808 | CE2 | PHE | A | 139 | 47.520 | 1.225 | 22.563 | 1.00 | 62.51 | C |
| ATOM | 809 | CZ | PHE | A | 139 | 48.675 | 1.787 | 23.072 | 1.00 | 67.11 | C |
| ATOM | 810 | N | ARG | A | 140 | 43.250 | 2.153 | 24.067 | 1.00 | 62.52 | N |
| ATOM | 811 | CA | ARG | A | 140 | 42.610 | 2.657 | 22.859 | 1.00 | 65.01 | C |
| ATOM | 812 | C | ARG | A | 140 | 41.910 | 3.981 | 23.147 | 1.00 | 54.59 | C |
| ATOM | 813 | O | ARG | A | 140 | 41.828 | 4.848 | 22.283 | 1.00 | 50.00 | O |
| ATOM | 814 | CB | ARG | A | 140 | 41.627 | 1.626 | 22.292 | 1.00 | 64.66 | C |
| ATOM | 815 | CG | ARG | A | 140 | 42.298 | 0.427 | 21.627 | 1.00 | 72.71 | C |
| ATOM | 816 | CD | ARG | A | 140 | 41.275 | −0.501 | 20.988 | 1.00 | 92.46 | C |
| ATOM | 817 | NE | ARG | A | 140 | 41.848 | −1.265 | 19.883 | 1.00 | 114.69 | N |
| ATOM | 818 | CZ | ARG | A | 140 | 41.138 | −2.003 | 19.034 | 1.00 | 128.89 | C |
| ATOM | 819 | NH1 | ARG | A | 140 | 39.820 | −2.082 | 19.164 | 1.00 | 121.02 | N |
| ATOM | 820 | NH2 | ARG | A | 140 | 41.746 | −2.662 | 18.052 | 1.00 | 132.20 | N |
| ATOM | 821 | N | TYR | A | 141 | 41.426 | 4.143 | 24.373 | 1.00 | 44.20 | N |
| ATOM | 822 | CA | TYR | A | 141 | 40.816 | 5.399 | 24.752 | 1.00 | 40.46 | C |
| ATOM | 823 | C | TYR | A | 141 | 41.834 | 6.528 | 24.845 | 1.00 | 47.63 | C |
| ATOM | 824 | O | TYR | A | 141 | 41.626 | 7.586 | 24.267 | 1.00 | 52.61 | O |
| ATOM | 825 | CB | TYR | A | 141 | 40.042 | 5.277 | 26.058 | 1.00 | 45.22 | C |
| ATOM | 826 | CG | TYR | A | 141 | 39.399 | 6.576 | 26.438 | 1.00 | 46.78 | C |
| ATOM | 827 | CD1 | TYR | A | 141 | 38.080 | 6.836 | 26.116 | 1.00 | 50.94 | C |
| ATOM | 828 | CD2 | TYR | A | 141 | 40.125 | 7.561 | 27.082 | 1.00 | 50.04 | C |
| ATOM | 829 | CE1 | TYR | A | 141 | 37.494 | 8.039 | 26.451 | 1.00 | 58.26 | C |
| ATOM | 830 | CE2 | TYR | A | 141 | 39.553 | 8.761 | 27.419 | 1.00 | 56.11 | C |
| ATOM | 831 | CZ | TYR | A | 141 | 38.239 | 8.999 | 27.104 | 1.00 | 58.99 | C |
| ATOM | 832 | OH | TYR | A | 141 | 37.674 | 10.207 | 27.445 | 1.00 | 67.52 | O |
| ATOM | 833 | N | GLN | A | 142 | 42.927 | 6.313 | 25.573 | 1.00 | 60.74 | N |
| ATOM | 834 | CA | GLN | A | 142 | 43.962 | 7.346 | 25.709 | 1.00 | 61.14 | C |
| ATOM | 835 | C | GLN | A | 142 | 44.581 | 7.704 | 24.359 | 1.00 | 50.67 | C |
| ATOM | 836 | O | GLN | A | 142 | 45.029 | 8.831 | 24.150 | 1.00 | 48.73 | O |
| ATOM | 837 | CB | GLN | A | 142 | 45.070 | 6.928 | 26.688 | 1.00 | 43.75 | C |
| ATOM | 838 | CG | GLN | A | 142 | 44.635 | 6.778 | 28.135 | 1.00 | 66.68 | C |
| ATOM | 839 | CD | GLN | A | 142 | 44.155 | 8.087 | 28.750 | 1.00 | 85.90 | C |
| ATOM | 840 | OE1 | GLN | A | 142 | 44.232 | 9.144 | 28.121 | 1.00 | 83.34 | O |
| ATOM | 841 | NE2 | GLN | A | 142 | 43.653 | 8.020 | 29.987 | 1.00 | 78.62 | N |
| ATOM | 842 | N | SER | A | 143 | 44.603 | 6.747 | 23.441 | 1.00 | 39.96 | N |
| ATOM | 843 | CA | SER | A | 143 | 45.270 | 6.974 | 22.164 | 1.00 | 59.02 | C |
| ATOM | 844 | C | SER | A | 143 | 44.382 | 7.607 | 21.077 | 1.00 | 57.13 | C |
| ATOM | 845 | O | SER | A | 143 | 44.893 | 8.197 | 20.128 | 1.00 | 52.88 | O |
| ATOM | 846 | CB | SER | A | 143 | 45.942 | 5.692 | 21.658 | 1.00 | 58.12 | C |
| ATOM | 847 | OG | SER | A | 143 | 45.057 | 4.588 | 21.683 | 1.00 | 66.04 | O |
| ATOM | 848 | N | LEU | A | 144 | 43.065 | 7.503 | 21.219 | 1.00 | 54.42 | N |
| ATOM | 849 | CA | LEU | A | 144 | 42.153 | 8.080 | 20.228 | 1.00 | 49.61 | C |
| ATOM | 850 | C | LEU | A | 144 | 41.529 | 9.399 | 20.665 | 1.00 | 52.78 | C |
| ATOM | 851 | O | LEU | A | 144 | 41.476 | 10.346 | 19.887 | 1.00 | 43.31 | O |
| ATOM | 852 | CB | LEU | A | 144 | 41.043 | 7.097 | 19.867 | 1.00 | 44.97 | C |
| ATOM | 853 | CG | LEU | A | 144 | 41.571 | 5.805 | 19.263 | 1.00 | 58.94 | C |
| ATOM | 854 | CD1 | LEU | A | 144 | 40.456 | 4.771 | 19.163 | 1.00 | 39.40 | C |
| ATOM | 855 | CD2 | LEU | A | 144 | 42.216 | 6.096 | 17.913 | 1.00 | 44.96 | C |
| ATOM | 856 | N | MET | A | 145 | 41.042 | 9.462 | 21.901 | 1.00 | 54.67 | N |
| ATOM | 857 | CA | MET | A | 145 | 40.340 | 10.659 | 22.359 | 1.00 | 51.28 | C |
| ATOM | 858 | C | MET | A | 145 | 41.272 | 11.802 | 22.735 | 1.00 | 43.78 | C |
| ATOM | 859 | O | MET | A | 145 | 42.127 | 11.676 | 23.607 | 1.00 | 53.96 | O |
| ATOM | 860 | CB | MET | A | 145 | 39.349 | 10.339 | 23.486 | 1.00 | 56.60 | C |
| ATOM | 861 | CG | MET | A | 145 | 38.009 | 9.785 | 22.967 | 1.00 | 73.46 | C |
| ATOM | 862 | SD | MET | A | 145 | 36.685 | 10.998 | 22.701 | 1.00 | 67.92 | S |
| ATOM | 863 | CE | MET | A | 145 | 37.590 | 12.542 | 22.747 | 1.00 | 40.65 | C |
| ATOM | 864 | N | THR | A | 146 | 41.091 | 12.919 | 22.047 | 1.00 | 38.95 | N |
| ATOM | 865 | CA | THR | A | 146 | 41.873 | 14.117 | 22.283 | 1.00 | 39.32 | C |
| ATOM | 866 | C | THR | A | 146 | 40.912 | 15.282 | 22.381 | 1.00 | 33.90 | C |
| ATOM | 867 | O | THR | A | 146 | 39.720 | 15.119 | 22.148 | 1.00 | 48.32 | O |
| ATOM | 868 | CB | THR | A | 146 | 42.864 | 14.377 | 21.120 | 1.00 | 47.11 | C |
| ATOM | 869 | OG1 | THR | A | 146 | 42.145 | 14.666 | 19.910 | 1.00 | 36.97 | O |
| ATOM | 870 | CG2 | THR | A | 146 | 43.727 | 13.159 | 20.893 | 1.00 | 40.04 | C |
| ATOM | 871 | N | ARG | A | 147 | 41.435 | 16.464 | 22.687 | 1.00 | 38.18 | N |
| ATOM | 872 | CA | ARG | A | 147 | 40.603 | 17.654 | 22.847 | 1.00 | 43.44 | C |
| ATOM | 873 | C | ARG | A | 147 | 40.028 | 18.125 | 21.518 | 1.00 | 34.57 | C |
| ATOM | 874 | O | ARG | A | 147 | 38.885 | 18.562 | 21.437 | 1.00 | 39.09 | O |
| ATOM | 875 | CB | ARG | A | 147 | 41.401 | 18.789 | 23.493 | 1.00 | 42.59 | C |
| ATOM | 876 | CG | ARG | A | 147 | 41.543 | 18.683 | 24.999 | 1.00 | 42.52 | C |
| ATOM | 877 | CD | ARG | A | 147 | 42.184 | 19.938 | 25.549 | 1.00 | 53.25 | C |
| ATOM | 878 | NE | ARG | A | 147 | 41.647 | 21.124 | 24.886 | 1.00 | 72.74 | N |
| ATOM | 879 | CZ | ARG | A | 147 | 40.785 | 21.972 | 25.444 | 1.00 | 87.64 | C |
| ATOM | 880 | NH1 | ARG | A | 147 | 40.368 | 21.772 | 26.691 | 1.00 | 62.96 | N |
| ATOM | 881 | NH2 | ARG | A | 147 | 40.346 | 23.027 | 24.760 | 1.00 | 81.57 | N |
| ATOM | 882 | N | ALA | A | 148 | 40.832 | 18.043 | 20.473 | 1.00 | 40.70 | N |
| ATOM | 883 | CA | ALA | A | 148 | 40.380 | 18.440 | 19.147 | 1.00 | 40.53 | C |
| ATOM | 884 | C | ALA | A | 148 | 39.252 | 17.527 | 18.702 | 1.00 | 40.03 | C |
| ATOM | 885 | O | ALA | A | 148 | 38.280 | 17.974 | 18.088 | 1.00 | 33.02 | O |
| ATOM | 886 | CB | ALA | A | 148 | 41.527 | 18.376 | 18.161 | 1.00 | 27.09 | C |
| ATOM | 887 | N | ARG | A | 149 | 39.398 | 16.244 | 19.023 | 1.00 | 33.11 | N |
| ATOM | 888 | CA | ARG | A | 149 | 38.413 | 15.251 | 18.654 | 1.00 | 28.79 | C |
| ATOM | 889 | C | ARG | A | 149 | 37.094 | 15.460 | 19.375 | 1.00 | 34.20 | C |
| ATOM | 890 | O | ARG | A | 149 | 36.022 | 15.474 | 18.754 | 1.00 | 30.21 | O |
| ATOM | 891 | CB | ARG | A | 149 | 38.952 | 13.844 | 18.901 | 1.00 | 34.28 | C |
| ATOM | 892 | CG | ARG | A | 149 | 39.189 | 13.106 | 17.606 | 1.00 | 27.64 | C |
| ATOM | 893 | CD | ARG | A | 149 | 40.069 | 11.892 | 17.735 | 1.00 | 36.04 | C |
| ATOM | 894 | NE | ARG | A | 149 | 41.030 | 11.886 | 16.635 | 1.00 | 45.94 | N |
| ATOM | 895 | CZ | ARG | A | 149 | 42.053 | 11.050 | 16.526 | 1.00 | 47.53 | C |
| ATOM | 896 | NH1 | ARG | A | 149 | 42.259 | 10.116 | 17.443 | 1.00 | 54.10 | N |
| ATOM | 897 | NH2 | ARG | A | 149 | 42.868 | 11.148 | 15.491 | 1.00 | 50.77 | N |
| ATOM | 898 | N | ALA | A | 150 | 37.180 | 15.622 | 20.687 | 1.00 | 33.01 | N |
| ATOM | 899 | CA | ALA | A | 150 | 35.999 | 15.881 | 21.500 | 1.00 | 31.61 | C |
| ATOM | 900 | C | ALA | A | 150 | 35.179 | 17.063 | 20.963 | 1.00 | 32.79 | C |
| ATOM | 901 | O | ALA | A | 150 | 33.948 | 16.996 | 20.925 | 1.00 | 29.03 | O |
| ATOM | 902 | CB | ALA | A | 150 | 36.402 | 16.112 | 22.934 | 1.00 | 27.20 | C |
| ATOM | 903 | N | LYS | A | 151 | 35.864 | 18.131 | 20.544 | 1.00 | 26.42 | N |
| ATOM | 904 | CA | LYS | A | 151 | 35.206 | 19.309 | 19.976 | 1.00 | 30.25 | C |
| ATOM | 905 | C | LYS | A | 151 | 34.480 | 18.980 | 18.676 | 1.00 | 36.13 | C |
| ATOM | 906 | O | LYS | A | 151 | 33.372 | 19.474 | 18.422 | 1.00 | 29.92 | O |
| ATOM | 907 | CB | LYS | A | 151 | 36.203 | 20.450 | 19.763 | 1.00 | 25.66 | C |
| ATOM | 908 | CG | LYS | A | 151 | 36.644 | 21.080 | 21.070 | 1.00 | 49.85 | C |
| ATOM | 909 | CD | LYS | A | 151 | 37.619 | 22.227 | 20.865 | 1.00 | 62.10 | C |
| ATOM | 910 | CE | LYS | A | 151 | 38.486 | 22.431 | 22.113 | 1.00 | 54.49 | C |
| ATOM | 911 | NZ | LYS | A | 151 | 39.246 | 23.717 | 22.076 | 1.00 | 67.73 | N |
| ATOM | 912 | N | VAL | A | 152 | 35.104 | 18.140 | 17.857 | 1.00 | 25.42 | N |
| ATOM | 913 | CA | VAL | A | 152 | 34.441 | 17.653 | 16.663 | 1.00 | 28.28 | C |
| ATOM | 914 | C | VAL | A | 152 | 33.142 | 16.954 | 17.067 | 1.00 | 25.71 | C |
| ATOM | 915 | O | VAL | A | 152 | 32.084 | 17.245 | 16.519 | 1.00 | 31.29 | O |
| ATOM | 916 | CB | VAL | A | 152 | 35.374 | 16.747 | 15.807 | 1.00 | 25.73 | C |
| ATOM | 917 | CG1 | VAL | A | 152 | 34.596 | 15.941 | 14.780 | 1.00 | 16.62 | C |
| ATOM | 918 | CG2 | VAL | A | 152 | 36.390 | 17.602 | 15.106 | 1.00 | 26.38 | C |
| ATOM | 919 | N | ILE | A | 153 | 33.217 | 16.060 | 18.045 | 1.00 | 24.25 | N |
| ATOM | 920 | CA | ILE | A | 153 | 32.032 | 15.356 | 18.531 | 1.00 | 25.84 | C |
| ATOM | 921 | C | ILE | A | 153 | 30.935 | 16.295 | 19.059 | 1.00 | 25.44 | C |
| ATOM | 922 | O | ILE | A | 153 | 29.753 | 16.092 | 18.786 | 1.00 | 28.98 | O |
| ATOM | 923 | CB | ILE | A | 153 | 32.394 | 14.336 | 19.627 | 1.00 | 26.23 | C |
| ATOM | 924 | CG1 | ILE | A | 153 | 33.361 | 13.291 | 19.079 | 1.00 | 20.10 | C |
| ATOM | 925 | CG2 | ILE | A | 153 | 31.124 | 13.675 | 20.174 | 1.00 | 21.37 | C |
| ATOM | 926 | CD1 | ILE | A | 153 | 34.241 | 12.652 | 20.136 | 1.00 | 16.51 | C |
| ATOM | 927 | N | ILE | A | 154 | 31.325 | 17.313 | 19.814 | 1.00 | 19.46 | N |
| ATOM | 928 | CA | ILE | A | 154 | 30.377 | 18.304 | 20.302 | 1.00 | 25.47 | C |
| ATOM | 929 | C | ILE | A | 154 | 29.584 | 18.924 | 19.146 | 1.00 | 29.16 | C |
| ATOM | 930 | O | ILE | A | 154 | 28.354 | 19.036 | 19.204 | 1.00 | 25.93 | O |
| ATOM | 931 | CB | ILE | A | 154 | 31.097 | 19.406 | 21.133 | 1.00 | 28.95 | C |
| ATOM | 932 | CG1 | ILE | A | 154 | 31.327 | 18.914 | 22.560 | 1.00 | 30.02 | C |
| ATOM | 933 | CG2 | ILE | A | 154 | 30.300 | 20.712 | 21.162 | 1.00 | 17.91 | C |
| ATOM | 934 | CD1 | ILE | A | 154 | 32.452 | 19.617 | 23.273 | 1.00 | 40.22 | C |
| ATOM | 935 | N | CYS | A | 155 | 30.292 | 19.304 | 18.090 | 1.00 | 22.97 | N |
| ATOM | 936 | CA | CYS | A | 155 | 29.688 | 20.029 | 16.980 | 1.00 | 26.86 | C |
| ATOM | 937 | C | CYS | A | 155 | 28.842 | 19.124 | 16.105 | 1.00 | 27.20 | C |
| ATOM | 938 | O | CYS | A | 155 | 27.834 | 19.546 | 15.548 | 1.00 | 21.85 | O |
| ATOM | 939 | CB | CYS | A | 155 | 30.771 | 20.704 | 16.130 | 1.00 | 46.42 | C |
| ATOM | 940 | SG | CYS | A | 155 | 31.803 | 21.872 | 17.055 | 1.00 | 62.56 | S |
| ATOM | 941 | N | THR | A | 156 | 29.278 | 17.884 | 15.964 | 1.00 | 26.28 | N |
| ATOM | 942 | CA | THR | A | 156 | 28.499 | 16.876 | 15.274 | 1.00 | 27.43 | C |
| ATOM | 943 | C | THR | A | 156 | 27.156 | 16.735 | 15.994 | 1.00 | 22.78 | C |
| ATOM | 944 | O | THR | A | 156 | 26.109 | 16.695 | 15.354 | 1.00 | 23.07 | O |
| ATOM | 945 | CB | THR | A | 156 | 29.267 | 15.525 | 15.233 | 1.00 | 24.67 | C |
| ATOM | 946 | OG1 | THR | A | 156 | 30.516 | 15.725 | 14.573 | 1.00 | 32.61 | O |
| ATOM | 947 | CG2 | THR | A | 156 | 28.499 | 14.454 | 14.487 | 1.00 | 16.93 | C |
| ATOM | 948 | N | VAL | A | 157 | 27.196 | 16.683 | 17.323 | 1.00 | 22.85 | N |
| ATOM | 949 | CA | VAL | A | 157 | 25.986 | 16.642 | 18.135 | 1.00 | 19.97 | C |
| ATOM | 950 | C | VAL | A | 157 | 25.076 | 17.855 | 17.938 | 1.00 | 20.65 | C |
| ATOM | 951 | O | VAL | A | 157 | 23.886 | 17.696 | 17.714 | 1.00 | 23.98 | O |
| ATOM | 952 | CB | VAL | A | 157 | 26.321 | 16.492 | 19.609 | 1.00 | 19.03 | C |
| ATOM | 953 | CG1 | VAL | A | 157 | 25.172 | 17.006 | 20.455 | 1.00 | 28.71 | C |
| ATOM | 954 | CG2 | VAL | A | 157 | 26.606 | 15.037 | 19.927 | 1.00 | 20.93 | C |
| ATOM | 955 | N | TRP | A | 158 | 25.630 | 19.061 | 18.012 | 1.00 | 22.27 | N |
| ATOM | 956 | CA | TRP | A | 158 | 24.857 | 20.275 | 17.747 | 1.00 | 19.73 | C |
| ATOM | 957 | C | TRP | A | 158 | 24.307 | 20.339 | 16.310 | 1.00 | 24.62 | C |
| ATOM | 958 | O | TRP | A | 158 | 23.186 | 20.804 | 16.078 | 1.00 | 21.21 | O |
| ATOM | 959 | CB | TRP | A | 158 | 25.688 | 21.522 | 18.080 | 1.00 | 18.83 | C |
| ATOM | 960 | CG | TRP | A | 158 | 25.735 | 21.824 | 19.559 | 1.00 | 33.40 | C |
| ATOM | 961 | CD1 | TRP | A | 158 | 26.697 | 21.433 | 20.451 | 1.00 | 32.62 | C |
| ATOM | 962 | CD2 | TRP | A | 158 | 24.775 | 22.574 | 20.314 | 1.00 | 33.09 | C |
| ATOM | 963 | NE1 | TRP | A | 158 | 26.393 | 21.890 | 21.712 | 1.00 | 24.67 | N |
| ATOM | 964 | CE2 | TRP | A | 158 | 25.221 | 22.592 | 21.657 | 1.00 | 29.13 | C |
| ATOM | 965 | CE3 | TRP | A | 158 | 23.583 | 23.231 | 19.988 | 1.00 | 32.10 | C |
| ATOM | 966 | CZ2 | TRP | A | 158 | 24.520 | 23.243 | 22.671 | 1.00 | 33.07 | C |
| ATOM | 967 | CZ3 | TRP | A | 158 | 22.883 | 23.877 | 21.001 | 1.00 | 41.35 | C |
| ATOM | 968 | CH2 | TRP | A | 158 | 23.356 | 23.878 | 22.329 | 1.00 | 33.80 | C |
| ATOM | 969 | N | ALA | A | 159 | 25.103 | 19.878 | 15.345 | 1.00 | 29.11 | N |
| ATOM | 970 | CA | ALA | A | 159 | 24.672 | 19.775 | 13.949 | 1.00 | 19.78 | C |
| ATOM | 971 | C | ALA | A | 159 | 23.450 | 18.876 | 13.811 | 1.00 | 24.84 | C |
| ATOM | 972 | O | ALA | A | 159 | 22.405 | 19.309 | 13.319 | 1.00 | 23.96 | O |
| ATOM | 973 | CB | ALA | A | 159 | 25.780 | 19.226 | 13.107 | 1.00 | 20.95 | C |
| ATOM | 974 | N | ILE | A | 160 | 23.601 | 17.620 | 14.229 | 1.00 | 17.70 | N |
| ATOM | 975 | CA | ILE | A | 160 | 22.496 | 16.672 | 14.219 | 1.00 | 21.33 | C |
| ATOM | 976 | C | ILE | A | 160 | 21.284 | 17.252 | 14.906 | 1.00 | 26.70 | C |
| ATOM | 977 | O | ILE | A | 160 | 20.145 | 17.048 | 14.479 | 1.00 | 26.14 | O |
| ATOM | 978 | CB | ILE | A | 160 | 22.866 | 15.354 | 14.901 | 1.00 | 26.99 | C |
| ATOM | 979 | CG1 | ILE | A | 160 | 23.926 | 14.627 | 14.066 | 1.00 | 28.62 | C |
| ATOM | 980 | CG2 | ILE | A | 160 | 21.628 | 14.493 | 15.089 | 1.00 | 18.16 | C |
| ATOM | 981 | CD1 | ILE | A | 160 | 24.569 | 13.445 | 14.770 | 1.00 | 21.61 | C |
| ATOM | 982 | N | SER | A | 161 | 21.534 | 17.999 | 15.968 | 1.00 | 24.93 | N |
| ATOM | 983 | CA | SER | A | 161 | 20.451 | 18.604 | 16.729 | 1.00 | 26.83 | C |
| ATOM | 984 | C | SER | A | 161 | 19.682 | 19.686 | 15.965 | 1.00 | 20.50 | C |
| ATOM | 985 | O | SER | A | 161 | 18.462 | 19.649 | 15.946 | 1.00 | 29.11 | O |
| ATOM | 986 | CB | SER | A | 161 | 20.969 | 19.129 | 18.066 | 1.00 | 29.32 | C |
| ATOM | 987 | OG | SER | A | 161 | 21.426 | 18.054 | 18.865 | 1.00 | 30.79 | O |
| ATOM | 988 | N | ALA | A | 162 | 20.376 | 20.642 | 15.348 | 1.00 | 18.91 | N |
| ATOM | 989 | CA | ALA | A | 162 | 19.706 | 21.626 | 14.489 | 1.00 | 27.30 | C |
| ATOM | 990 | C | ALA | A | 162 | 18.953 | 20.965 | 13.305 | 1.00 | 30.30 | C |
| ATOM | 991 | O | ALA | A | 162 | 17.845 | 21.369 | 12.948 | 1.00 | 25.01 | O |
| ATOM | 992 | CB | ALA | A | 162 | 20.699 | 22.660 | 13.991 | 1.00 | 16.13 | C |
| ATOM | 993 | N | LEU | A | 163 | 19.568 | 19.952 | 12.704 | 1.00 | 23.68 | N |
| ATOM | 994 | CA | LEU | A | 163 | 18.916 | 19.143 | 11.689 | 1.00 | 26.16 | C |
| ATOM | 995 | C | LEU | A | 163 | 17.496 | 18.688 | 12.086 | 1.00 | 25.27 | C |
| ATOM | 996 | O | LEU | A | 163 | 16.530 | 19.025 | 11.410 | 1.00 | 24.44 | O |
| ATOM | 997 | CB | LEU | A | 163 | 19.773 | 17.915 | 11.374 | 1.00 | 28.68 | C |
| ATOM | 998 | CG | LEU | A | 163 | 19.275 | 17.108 | 10.180 | 1.00 | 20.52 | C |
| ATOM | 999 | CD1 | LEU | A | 163 | 19.145 | 18.042 | 9.002 | 1.00 | 14.46 | C |
| ATOM | 1000 | CD2 | LEU | A | 163 | 20.205 | 15.952 | 9.892 | 1.00 | 16.80 | C |
| ATOM | 1001 | N | VAL | A | 164 | 17.380 | 17.909 | 13.160 | 1.00 | 28.53 | N |
| ATOM | 1002 | CA | VAL | A | 164 | 16.083 | 17.364 | 13.599 | 1.00 | 32.39 | C |
| ATOM | 1003 | C | VAL | A | 164 | 15.200 | 18.340 | 14.381 | 1.00 | 27.04 | C |
| ATOM | 1004 | O | VAL | A | 164 | 14.099 | 17.971 | 14.803 | 1.00 | 32.15 | O |
| ATOM | 1005 | CB | VAL | A | 164 | 16.229 | 16.067 | 14.455 | 1.00 | 28.86 | C |
| ATOM | 1006 | CG1 | VAL | A | 164 | 17.227 | 15.119 | 13.825 | 1.00 | 20.66 | C |
| ATOM | 1007 | CG2 | VAL | A | 164 | 16.620 | 16.396 | 15.893 | 1.00 | 17.30 | C |
| ATOM | 1008 | N | SER | A | 165 | 15.663 | 19.573 | 14.563 | 1.00 | 20.24 | N |
| ATOM | 1009 | CA | SER | A | 165 | 14.873 | 20.570 | 15.282 | 1.00 | 20.64 | C |
| ATOM | 1010 | C | SER | A | 165 | 14.560 | 21.863 | 14.502 | 1.00 | 24.66 | C |
| ATOM | 1011 | O | SER | A | 165 | 13.401 | 22.262 | 14.410 | 1.00 | 33.28 | O |
| ATOM | 1012 | CB | SER | A | 165 | 15.528 | 20.881 | 16.624 | 1.00 | 27.55 | C |
| ATOM | 1013 | OG | SER | A | 165 | 16.804 | 21.453 | 16.417 | 1.00 | 43.75 | O |
| ATOM | 1014 | N | PHE | A | 166 | 15.572 | 22.519 | 13.943 | 1.00 | 24.24 | N |
| ATOM | 1015 | CA | PHE | A | 166 | 15.323 | 23.676 | 13.070 | 1.00 | 32.73 | C |
| ATOM | 1016 | C | PHE | A | 166 | 14.622 | 23.336 | 11.749 | 1.00 | 33.79 | C |
| ATOM | 1017 | O | PHE | A | 166 | 13.695 | 24.028 | 11.325 | 1.00 | 26.90 | O |
| ATOM | 1018 | CB | PHE | A | 166 | 16.621 | 24.382 | 12.720 | 1.00 | 32.56 | C |
| ATOM | 1019 | CG | PHE | A | 166 | 17.183 | 25.175 | 13.831 | 1.00 | 36.86 | C |
| ATOM | 1020 | CD1 | PHE | A | 166 | 18.557 | 25.337 | 13.960 | 1.00 | 42.13 | C |
| ATOM | 1021 | CD2 | PHE | A | 166 | 16.346 | 25.750 | 14.760 | 1.00 | 27.66 | C |
| ATOM | 1022 | CE1 | PHE | A | 166 | 19.086 | 26.083 | 15.004 | 1.00 | 49.22 | C |
| ATOM | 1023 | CE2 | PHE | A | 166 | 16.855 | 26.495 | 15.803 | 1.00 | 38.43 | C |
| ATOM | 1024 | CZ | PHE | A | 166 | 18.226 | 26.663 | 15.932 | 1.00 | 48.77 | C |
| ATOM | 1025 | N | LEU | A | 167 | 15.079 | 22.276 | 11.095 | 1.00 | 29.01 | N |
| ATOM | 1026 | CA | LEU | A | 167 | 14.688 | 22.029 | 9.721 | 1.00 | 24.00 | C |
| ATOM | 1027 | C | LEU | A | 167 | 13.249 | 21.591 | 9.475 | 1.00 | 24.04 | C |
| ATOM | 1028 | O | LEU | A | 167 | 12.648 | 22.020 | 8.498 | 1.00 | 27.98 | O |
| ATOM | 1029 | CB | LEU | A | 167 | 15.692 | 21.113 | 9.028 | 1.00 | 28.26 | C |
| ATOM | 1030 | CG | LEU | A | 167 | 16.711 | 21.960 | 8.264 | 1.00 | 31.54 | C |
| ATOM | 1031 | CD1 | LEU | A | 167 | 18.045 | 21.265 | 8.175 | 1.00 | 25.53 | C |
| ATOM | 1032 | CD2 | LEU | A | 167 | 16.168 | 22.307 | 6.879 | 1.00 | 27.18 | C |
| ATOM | 1033 | N | PRO | A | 168 | 12.687 | 20.732 | 10.339 | 1.00 | 27.75 | N |
| ATOM | 1034 | CA | PRO | A | 168 | 11.246 | 20.464 | 10.183 | 1.00 | 26.79 | C |
| ATOM | 1035 | C | PRO | A | 168 | 10.353 | 21.669 | 10.525 | 1.00 | 24.39 | C |
| ATOM | 1036 | O | PRO | A | 168 | 9.212 | 21.747 | 10.063 | 1.00 | 20.81 | O |
| ATOM | 1037 | CB | PRO | A | 168 | 11.005 | 19.312 | 11.151 | 1.00 | 21.91 | C |
| ATOM | 1038 | CG | PRO | A | 168 | 12.345 | 18.633 | 11.234 | 1.00 | 24.33 | C |
| ATOM | 1039 | CD | PRO | A | 168 | 13.330 | 19.755 | 11.226 | 1.00 | 23.53 | C |
| ATOM | 1040 | N | ILE | A | 169 | 10.878 | 22.608 | 11.305 | 1.00 | 19.04 | N |
| ATOM | 1041 | CA | ILE | A | 169 | 10.124 | 23.811 | 11.606 | 1.00 | 20.73 | C |
| ATOM | 1042 | C | ILE | A | 169 | 10.129 | 24.783 | 10.434 | 1.00 | 24.70 | C |
| ATOM | 1043 | O | ILE | A | 169 | 9.094 | 25.359 | 10.098 | 1.00 | 33.23 | O |
| ATOM | 1044 | CB | ILE | A | 169 | 10.583 | 24.482 | 12.919 | 1.00 | 26.19 | C |
| ATOM | 1045 | CG1 | ILE | A | 169 | 10.250 | 23.557 | 14.086 | 1.00 | 28.71 | C |
| ATOM | 1046 | CG2 | ILE | A | 169 | 9.914 | 25.830 | 13.106 | 1.00 | 17.62 | C |
| ATOM | 1047 | CD1 | ILE | A | 169 | 9.801 | 24.266 | 15.320 | 1.00 | 35.25 | C |
| ATOM | 1048 | N | MET | A | 170 | 11.276 | 24.956 | 9.790 | 1.00 | 28.27 | N |
| ATOM | 1049 | CA | MET | A | 170 | 11.323 | 25.824 | 8.616 | 1.00 | 28.46 | C |
| ATOM | 1050 | C | MET | A | 170 | 10.593 | 25.255 | 7.374 | 1.00 | 26.77 | C |
| ATOM | 1051 | O | MET | A | 170 | 10.160 | 26.011 | 6.510 | 1.00 | 33.14 | O |
| ATOM | 1052 | CB | MET | A | 170 | 12.757 | 26.328 | 8.324 | 1.00 | 27.49 | C |
| ATOM | 1053 | CG | MET | A | 170 | 13.902 | 25.370 | 8.644 | 1.00 | 30.20 | C |
| ATOM | 1054 | SD | MET | A | 170 | 15.522 | 26.149 | 8.984 | 1.00 | 42.10 | S |
| ATOM | 1055 | CE | MET | A | 170 | 15.607 | 27.508 | 7.825 | 1.00 | 23.28 | C |
| ATOM | 1056 | N | MET | A | 171 | 10.428 | 23.939 | 7.298 | 1.00 | 22.30 | N |
| ATOM | 1057 | CA | MET | A | 171 | 9.645 | 23.330 | 6.225 | 1.00 | 23.82 | C |
| ATOM | 1058 | C | MET | A | 171 | 8.202 | 23.080 | 6.651 | 1.00 | 28.63 | C |
| ATOM | 1059 | O | MET | A | 171 | 7.449 | 22.401 | 5.963 | 1.00 | 22.06 | O |
| ATOM | 1060 | CB | MET | A | 171 | 10.278 | 22.025 | 5.780 | 1.00 | 19.47 | C |
| ATOM | 1061 | CG | MET | A | 171 | 11.685 | 22.199 | 5.237 | 1.00 | 28.76 | C |
| ATOM | 1062 | SD | MET | A | 171 | 12.498 | 20.601 | 5.081 | 1.00 | 36.21 | S |
| ATOM | 1063 | CE | MET | A | 171 | 11.485 | 19.901 | 3.773 | 1.00 | 63.05 | C |
| ATOM | 1064 | N | HIS | A | 172 | 7.837 | 23.612 | 7.808 | 1.00 | 23.06 | N |
| ATOM | 1065 | CA | HIS | A | 172 | 6.469 | 23.532 | 8.294 | 1.00 | 28.74 | C |
| ATOM | 1066 | C | HIS | A | 172 | 5.919 | 22.113 | 8.461 | 1.00 | 26.57 | C |
| ATOM | 1067 | O | HIS | A | 172 | 4.716 | 21.909 | 8.381 | 1.00 | 30.69 | O |
| ATOM | 1068 | CB | HIS | A | 172 | 5.546 | 24.347 | 7.390 | 1.00 | 20.36 | C |
| ATOM | 1069 | CG | HIS | A | 172 | 6.039 | 25.737 | 7.123 | 1.00 | 21.94 | C |
| ATOM | 1070 | ND1 | HIS | A | 172 | 5.523 | 26.841 | 7.757 | 1.00 | 24.43 | N |
| ATOM | 1071 | CD2 | HIS | A | 172 | 6.992 | 26.193 | 6.281 | 1.00 | 25.54 | C |
| ATOM | 1072 | CE1 | HIS | A | 172 | 6.149 | 27.926 | 7.326 | 1.00 | 24.60 | C |
| ATOM | 1073 | NE2 | HIS | A | 172 | 7.036 | 27.562 | 6.426 | 1.00 | 25.10 | N |
| ATOM | 1074 | N | TRP | A | 173 | 6.788 | 21.148 | 8.734 | 1.00 | 22.96 | N |
| ATOM | 1075 | CA | TRP | A | 173 | 6.374 | 19.747 | 8.827 | 1.00 | 24.25 | C |
| ATOM | 1076 | C | TRP | A | 173 | 5.522 | 19.491 | 10.059 | 1.00 | 31.27 | C |
| ATOM | 1077 | O | TRP | A | 173 | 4.900 | 18.439 | 10.190 | 1.00 | 32.37 | O |
| ATOM | 1078 | CB | TRP | A | 173 | 7.598 | 18.834 | 8.859 | 1.00 | 21.12 | C |
| ATOM | 1079 | CG | TRP | A | 173 | 8.265 | 18.658 | 7.535 | 1.00 | 23.88 | C |
| ATOM | 1080 | CD1 | TRP | A | 173 | 8.046 | 19.387 | 6.399 | 1.00 | 22.56 | C |
| ATOM | 1081 | CD2 | TRP | A | 173 | 9.286 | 17.710 | 7.210 | 1.00 | 28.30 | C |
| ATOM | 1082 | NE1 | TRP | A | 173 | 8.853 | 18.936 | 5.388 | 1.00 | 23.45 | N |
| ATOM | 1083 | CE2 | TRP | A | 173 | 9.624 | 17.905 | 5.857 | 1.00 | 26.74 | C |
| ATOM | 1084 | CE3 | TRP | A | 173 | 9.943 | 16.701 | 7.931 | 1.00 | 23.21 | C |
| ATOM | 1085 | CZ2 | TRP | A | 173 | 10.592 | 17.133 | 5.207 | 1.00 | 23.76 | C |
| ATOM | 1086 | CZ3 | TRP | A | 173 | 10.899 | 15.934 | 7.278 | 1.00 | 26.95 | C |
| ATOM | 1087 | CH2 | TRP | A | 173 | 11.214 | 16.154 | 5.932 | 1.00 | 17.99 | C |
| ATOM | 1088 | N | TRP | A | 174 | 5.489 | 20.465 | 10.959 | 1.00 | 28.57 | N |
| ATOM | 1089 | CA | TRP | A | 174 | 4.858 | 20.284 | 12.255 | 1.00 | 21.85 | C |
| ATOM | 1090 | C | TRP | A | 174 | 3.386 | 20.668 | 12.261 | 1.00 | 27.91 | C |
| ATOM | 1091 | O | TRP | A | 174 | 2.632 | 20.232 | 13.134 | 1.00 | 26.71 | O |
| ATOM | 1092 | CB | TRP | A | 174 | 5.591 | 21.112 | 13.302 | 1.00 | 22.27 | C |
| ATOM | 1093 | CG | TRP | A | 174 | 5.600 | 22.570 | 13.003 | 1.00 | 20.96 | C |
| ATOM | 1094 | CD1 | TRP | A | 174 | 6.434 | 23.224 | 12.145 | 1.00 | 22.59 | C |
| ATOM | 1095 | CD2 | TRP | A | 174 | 4.726 | 23.567 | 13.549 | 1.00 | 19.39 | C |
| ATOM | 1096 | NE1 | TRP | A | 174 | 6.139 | 24.568 | 12.125 | 1.00 | 19.11 | N |
| ATOM | 1097 | CE2 | TRP | A | 174 | 5.097 | 24.803 | 12.981 | 1.00 | 17.65 | C |
| ATOM | 1098 | CE3 | TRP | A | 174 | 3.663 | 23.533 | 14.456 | 1.00 | 20.83 | C |
| ATOM | 1099 | CZ2 | TRP | A | 174 | 4.453 | 25.990 | 13.300 | 1.00 | 18.18 | C |
| ATOM | 1100 | CZ3 | TRP | A | 174 | 3.031 | 24.717 | 14.773 | 1.00 | 18.72 | C |
| ATOM | 1101 | CH2 | TRP | A | 174 | 3.426 | 25.927 | 14.201 | 1.00 | 15.43 | C |
| ATOM | 1102 | N | ARG | A | 175 | 2.973 | 21.482 | 11.293 | 1.00 | 29.72 | N |
| ATOM | 1103 | CA | ARG | A | 175 | 1.633 | 22.078 | 11.319 | 1.00 | 35.08 | C |
| ATOM | 1104 | C | ARG | A | 175 | 0.459 | 21.091 | 11.156 | 1.00 | 35.34 | C |
| ATOM | 1105 | O | ARG | A | 175 | 0.580 | 20.058 | 10.508 | 1.00 | 27.63 | O |
| ATOM | 1106 | CB | ARG | A | 175 | 1.534 | 23.213 | 10.302 | 1.00 | 19.16 | C |
| ATOM | 1107 | CG | ARG | A | 175 | 2.477 | 24.340 | 10.578 | 1.00 | 17.50 | C |
| ATOM | 1108 | CD | ARG | A | 175 | 2.099 | 25.549 | 9.767 | 1.00 | 24.78 | C |
| ATOM | 1109 | NE | ARG | A | 175 | 3.179 | 26.520 | 9.644 | 1.00 | 18.77 | N |
| ATOM | 1110 | CZ | ARG | A | 175 | 3.331 | 27.565 | 10.448 | 1.00 | 25.99 | C |
| ATOM | 1111 | NH1 | ARG | A | 175 | 2.476 | 27.774 | 11.445 | 1.00 | 28.26 | N |
| ATOM | 1112 | NH2 | ARG | A | 175 | 4.339 | 28.406 | 10.258 | 1.00 | 27.58 | N |
| ATOM | 1113 | N | ASP | A | 176 | −0.678 | 21.425 | 11.760 | 1.00 | 42.01 | N |
| ATOM | 1114 | CA | ASP | A | 176 | −1.878 | 20.602 | 11.633 | 1.00 | 42.19 | C |
| ATOM | 1115 | C | ASP | A | 176 | −2.897 | 21.224 | 10.674 | 1.00 | 35.42 | C |
| ATOM | 1116 | O | ASP | A | 176 | −2.731 | 22.354 | 10.221 | 1.00 | 33.26 | O |
| ATOM | 1117 | CB | ASP | A | 176 | −2.504 | 20.347 | 13.004 | 1.00 | 35.28 | C |
| ATOM | 1118 | CG | ASP | A | 176 | −3.238 | 19.014 | 13.071 | 1.00 | 59.33 | C |
| ATOM | 1119 | OD1 | ASP | A | 176 | −3.276 | 18.278 | 12.052 | 1.00 | 49.89 | O |
| ATOM | 1120 | OD2 | ASP | A | 176 | −3.774 | 18.697 | 14.156 | 1.00 | 63.93 | O |
| ATOM | 1121 | N | GLU | A | 177 | −3.939 | 20.469 | 10.353 | 1.00 | 43.63 | N |
| ATOM | 1122 | CA | GLU | A | 177 | −4.968 | 20.943 | 9.435 | 1.00 | 46.14 | C |
| ATOM | 1123 | C | GLU | A | 177 | −6.194 | 21.532 | 10.159 | 1.00 | 47.04 | C |
| ATOM | 1124 | O | GLU | A | 177 | −6.865 | 22.414 | 9.620 | 1.00 | 47.13 | O |
| ATOM | 1125 | CB | GLU | A | 177 | −5.382 | 19.833 | 8.460 | 1.00 | 48.62 | C |
| ATOM | 1126 | CG | GLU | A | 177 | −4.227 | 19.159 | 7.707 | 1.00 | 53.09 | C |
| ATOM | 1127 | CD | GLU | A | 177 | −3.646 | 20.017 | 6.585 | 1.00 | 79.57 | C |
| ATOM | 1128 | OE1 | GLU | A | 177 | −4.037 | 21.203 | 6.469 | 1.00 | 78.39 | O |
| ATOM | 1129 | OE2 | GLU | A | 177 | −2.796 | 19.504 | 5.818 | 1.00 | 60.84 | O |
| ATOM | 1130 | N | ASP | A | 178 | −6.464 | 21.063 | 11.381 | 1.00 | 51.55 | N |
| ATOM | 1131 | CA | ASP | A | 178 | −7.569 | 21.575 | 12.229 | 1.00 | 58.72 | C |
| ATOM | 1132 | C | ASP | A | 178 | −7.781 | 23.093 | 12.243 | 1.00 | 55.59 | C |
| ATOM | 1133 | O | ASP | A | 178 | −6.826 | 23.869 | 12.315 | 1.00 | 46.01 | O |
| ATOM | 1134 | CB | ASP | A | 178 | −7.399 | 21.117 | 13.678 | 1.00 | 57.10 | C |
| ATOM | 1135 | CG | ASP | A | 178 | −7.612 | 19.631 | 13.849 | 1.00 | 99.36 | C |
| ATOM | 1136 | OD1 | ASP | A | 178 | −7.832 | 18.939 | 12.830 | 1.00 | 105.06 | O |
| ATOM | 1137 | OD2 | ASP | A | 178 | −7.557 | 19.154 | 15.005 | 1.00 | 120.64 | O |
| ATOM | 1138 | N | PRO | A | 179 | −9.052 | 23.518 | 12.238 | 1.00 | 60.41 | N |
| ATOM | 1139 | CA | PRO | A | 179 | −9.358 | 24.952 | 12.208 | 1.00 | 58.74 | C |
| ATOM | 1140 | C | PRO | A | 179 | −8.793 | 25.599 | 13.467 | 1.00 | 50.01 | C |
| ATOM | 1141 | O | PRO | A | 179 | −8.323 | 26.742 | 13.460 | 1.00 | 42.90 | O |
| ATOM | 1142 | CB | PRO | A | 179 | −10.896 | 24.993 | 12.237 | 1.00 | 57.53 | C |
| ATOM | 1143 | CG | PRO | A | 179 | −11.353 | 23.562 | 12.057 | 1.00 | 55.42 | C |
| ATOM | 1144 | CD | PRO | A | 179 | −10.240 | 22.701 | 12.536 | 1.00 | 54.12 | C |
| ATOM | 1145 | N | GLN | A | 180 | −8.852 | 24.845 | 14.555 | 1.00 | 45.56 | N |
| ATOM | 1146 | CA | GLN | A | 180 | −8.319 | 25.297 | 15.823 | 1.00 | 55.89 | C |
| ATOM | 1147 | C | GLN | A | 180 | −6.843 | 25.595 | 15.639 | 1.00 | 47.63 | C |
| ATOM | 1148 | O | GLN | A | 180 | −6.390 | 26.705 | 15.921 | 1.00 | 40.03 | O |
| ATOM | 1149 | CB | GLN | A | 180 | −8.538 | 24.229 | 16.895 | 1.00 | 61.65 | C |
| ATOM | 1150 | CG | GLN | A | 180 | −10.007 | 23.862 | 17.108 | 1.00 | 77.78 | C |
| ATOM | 1151 | CD | GLN | A | 180 | −10.753 | 24.859 | 17.985 | 1.00 | 90.62 | C |
| ATOM | 1152 | OE1 | GLN | A | 180 | −10.160 | 25.516 | 18.846 | 1.00 | 86.25 | O |
| ATOM | 1153 | NE2 | GLN | A | 180 | −12.065 | 24.967 | 17.775 | 1.00 | 91.59 | N |
| ATOM | 1154 | N | ALA | A | 181 | −6.103 | 24.605 | 15.144 | 1.00 | 46.94 | N |
| ATOM | 1155 | CA | ALA | A | 181 | −4.696 | 24.797 | 14.796 | 1.00 | 41.73 | C |
| ATOM | 1156 | C | ALA | A | 181 | −4.532 | 25.994 | 13.868 | 1.00 | 42.87 | C |
| ATOM | 1157 | O | ALA | A | 181 | −3.665 | 26.840 | 14.085 | 1.00 | 30.80 | O |
| ATOM | 1158 | CB | ALA | A | 181 | −4.148 | 23.555 | 14.134 | 1.00 | 40.75 | C |
| ATOM | 1159 | N | LEU | A | 182 | −5.383 | 26.052 | 12.841 | 1.00 | 40.09 | N |
| ATOM | 1160 | CA | LEU | A | 182 | −5.315 | 27.094 | 11.818 | 1.00 | 34.23 | C |
| ATOM | 1161 | C | LEU | A | 182 | −5.510 | 28.517 | 12.361 | 1.00 | 33.37 | C |
| ATOM | 1162 | O | LEU | A | 182 | −4.798 | 29.436 | 11.967 | 1.00 | 33.31 | O |
| ATOM | 1163 | CB | LEU | A | 182 | −6.288 | 26.788 | 10.675 | 1.00 | 29.95 | C |
| ATOM | 1164 | CG | LEU | A | 182 | −5.913 | 25.590 | 9.792 | 1.00 | 35.25 | C |
| ATOM | 1165 | CD1 | LEU | A | 182 | −6.829 | 25.503 | 8.601 | 1.00 | 35.01 | C |
| ATOM | 1166 | CD2 | LEU | A | 182 | −4.469 | 25.685 | 9.311 | 1.00 | 33.15 | C |
| ATOM | 1167 | N | LYS | A | 183 | −6.456 | 28.703 | 13.273 | 1.00 | 37.94 | N |
| ATOM | 1168 | CA | LYS | A | 183 | −6.622 | 30.010 | 13.901 | 1.00 | 40.85 | C |
| ATOM | 1169 | C | LYS | A | 183 | −5.400 | 30.412 | 14.750 | 1.00 | 42.49 | C |
| ATOM | 1170 | O | LYS | A | 183 | −5.041 | 31.586 | 14.801 | 1.00 | 38.70 | O |
| ATOM | 1171 | CB | LYS | A | 183 | −7.931 | 30.056 | 14.686 | 1.00 | 43.62 | C |
| ATOM | 1172 | CG | LYS | A | 183 | −9.120 | 29.663 | 13.813 | 1.00 | 62.64 | C |
| ATOM | 1173 | CD | LYS | A | 183 | −10.471 | 29.994 | 14.433 | 1.00 | 79.17 | C |
| ATOM | 1174 | CE | LYS | A | 183 | −11.606 | 29.477 | 13.553 | 1.00 | 70.40 | C |
| ATOM | 1175 | NZ | LYS | A | 183 | −12.944 | 29.901 | 14.039 | 1.00 | 77.84 | N |
| ATOM | 1176 | N | CYS | A | 184 | −4.752 | 29.438 | 15.388 | 1.00 | 35.78 | N |
| ATOM | 1177 | CA | CYS | A | 184 | −3.492 | 29.683 | 16.089 | 1.00 | 38.43 | C |
| ATOM | 1178 | C | CYS | A | 184 | −2.378 | 30.103 | 15.126 | 1.00 | 35.36 | C |
| ATOM | 1179 | O | CYS | A | 184 | −1.464 | 30.847 | 15.496 | 1.00 | 40.56 | O |
| ATOM | 1180 | CB | CYS | A | 184 | −3.047 | 28.444 | 16.877 | 1.00 | 41.23 | C |
| ATOM | 1181 | SG | CYS | A | 184 | −1.881 | 28.806 | 18.210 | 1.00 | 66.98 | S |
| ATOM | 1182 | N | TYR | A | 185 | −2.444 | 29.617 | 13.892 | 1.00 | 30.80 | N |
| ATOM | 1183 | CA | TYR | A | 185 | −1.462 | 30.018 | 12.894 | 1.00 | 30.68 | C |
| ATOM | 1184 | C | TYR | A | 185 | −1.705 | 31.441 | 12.397 | 1.00 | 39.42 | C |
| ATOM | 1185 | O | TYR | A | 185 | −0.758 | 32.131 | 12.025 | 1.00 | 42.41 | O |
| ATOM | 1186 | CB | TYR | A | 185 | −1.393 | 29.036 | 11.729 | 1.00 | 26.92 | C |
| ATOM | 1187 | CG | TYR | A | 185 | −1.090 | 27.623 | 12.156 | 1.00 | 28.58 | C |
| ATOM | 1188 | CD1 | TYR | A | 185 | −0.357 | 27.374 | 13.303 | 1.00 | 22.63 | C |
| ATOM | 1189 | CD2 | TYR | A | 185 | −1.532 | 26.534 | 11.407 | 1.00 | 32.54 | C |
| ATOM | 1190 | CE1 | TYR | A | 185 | −0.075 | 26.082 | 13.713 | 1.00 | 19.52 | C |
| ATOM | 1191 | CE2 | TYR | A | 185 | −1.246 | 25.235 | 11.799 | 1.00 | 31.49 | C |
| ATOM | 1192 | CZ | TYR | A | 185 | −0.515 | 25.017 | 12.955 | 1.00 | 29.41 | C |
| ATOM | 1193 | OH | TYR | A | 185 | −0.222 | 23.734 | 13.357 | 1.00 | 28.90 | O |
| ATOM | 1194 | N | GLN | A | 186 | −2.957 | 31.896 | 12.402 | 1.00 | 37.20 | N |
| ATOM | 1195 | CA | GLN | A | 186 | −3.216 | 33.301 | 12.073 | 1.00 | 36.36 | C |
| ATOM | 1196 | C | GLN | A | 186 | −2.957 | 34.289 | 13.224 | 1.00 | 39.86 | C |
| ATOM | 1197 | O | GLN | A | 186 | −2.350 | 35.334 | 13.009 | 1.00 | 41.13 | O |
| ATOM | 1198 | CB | GLN | A | 186 | −4.586 | 33.527 | 11.410 | 1.00 | 35.77 | C |
| ATOM | 1199 | CG | GLN | A | 186 | −5.634 | 32.434 | 11.614 | 1.00 | 53.37 | C |
| ATOM | 1200 | CD | GLN | A | 186 | −6.921 | 32.698 | 10.811 | 1.00 | 61.65 | C |
| ATOM | 1201 | OE1 | GLN | A | 186 | −6.930 | 33.507 | 9.877 | 1.00 | 62.63 | O |
| ATOM | 1202 | NE2 | GLN | A | 186 | −8.006 | 32.016 | 11.180 | 1.00 | 51.74 | N |
| ATOM | 1203 | N | ASP | A | 187 | −3.386 | 33.960 | 14.440 | 1.00 | 41.10 | N |
| ATOM | 1204 | CA | ASP | A | 187 | −3.095 | 34.827 | 15.581 | 1.00 | 45.23 | C |
| ATOM | 1205 | C | ASP | A | 187 | −1.588 | 34.866 | 15.851 | 1.00 | 44.68 | C |
| ATOM | 1206 | O | ASP | A | 187 | −0.966 | 33.831 | 16.110 | 1.00 | 42.14 | O |
| ATOM | 1207 | CB | ASP | A | 187 | −3.857 | 34.381 | 16.837 | 1.00 | 39.82 | C |
| ATOM | 1208 | CG | ASP | A | 187 | −4.037 | 35.516 | 17.864 | 1.00 | 51.97 | C |
| ATOM | 1209 | OD1 | ASP | A | 187 | −3.359 | 36.568 | 17.757 | 1.00 | 41.55 | O |
| ATOM | 1210 | OD2 | ASP | A | 187 | −4.874 | 35.348 | 18.782 | 1.00 | 50.39 | O |
| ATOM | 1211 | N | PRO | A | 188 | −0.991 | 36.065 | 15.769 | 1.00 | 39.86 | N |
| ATOM | 1212 | CA | PRO | A | 188 | 0.424 | 36.237 | 16.105 | 1.00 | 44.77 | C |
| ATOM | 1213 | C | PRO | A | 188 | 0.565 | 36.191 | 17.614 | 1.00 | 43.12 | C |
| ATOM | 1214 | O | PRO | A | 188 | 1.616 | 35.826 | 18.137 | 1.00 | 51.55 | O |
| ATOM | 1215 | CB | PRO | A | 188 | 0.751 | 37.642 | 15.588 | 1.00 | 31.78 | C |
| ATOM | 1216 | CG | PRO | A | 188 | −0.429 | 38.058 | 14.773 | 1.00 | 40.94 | C |
| ATOM | 1217 | CD | PRO | A | 188 | −1.600 | 37.320 | 15.318 | 1.00 | 37.11 | C |
| ATOM | 1218 | N | GLY | A | 189 | −0.512 | 36.555 | 18.297 | 1.00 | 31.80 | N |
| ATOM | 1219 | CA | GLY | A | 189 | −0.549 | 36.533 | 19.740 | 1.00 | 32.93 | C |
| ATOM | 1220 | C | GLY | A | 189 | −0.746 | 35.139 | 20.291 | 1.00 | 34.14 | C |
| ATOM | 1221 | O | GLY | A | 189 | −0.733 | 34.938 | 21.501 | 1.00 | 45.59 | O |
| ATOM | 1222 | N | CYS | A | 190 | −0.945 | 34.172 | 19.409 | 1.00 | 33.34 | N |
| ATOM | 1223 | CA | CYS | A | 190 | −0.975 | 32.790 | 19.844 | 1.00 | 35.33 | C |
| ATOM | 1224 | C | CYS | A | 190 | 0.304 | 32.064 | 19.432 | 1.00 | 36.09 | C |
| ATOM | 1225 | O | CYS | A | 190 | 0.658 | 32.000 | 18.254 | 1.00 | 35.20 | O |
| ATOM | 1226 | CB | CYS | A | 190 | −2.204 | 32.068 | 19.312 | 1.00 | 24.66 | C |
| ATOM | 1227 | SG | CYS | A | 190 | −2.130 | 30.297 | 19.610 | 1.00 | 51.09 | S |
| ATOM | 1228 | N | CYS | A | 191 | 1.008 | 31.534 | 20.420 | 1.00 | 33.49 | N |
| ATOM | 1229 | CA | CYS | A | 191 | 2.246 | 30.827 | 20.166 | 1.00 | 29.17 | C |
| ATOM | 1230 | C | CYS | A | 191 | 2.179 | 29.438 | 20.756 | 1.00 | 31.76 | C |
| ATOM | 1231 | O | CYS | A | 191 | 3.043 | 29.038 | 21.533 | 1.00 | 37.94 | O |
| ATOM | 1232 | CB | CYS | A | 191 | 3.443 | 31.567 | 20.753 | 1.00 | 28.32 | C |
| ATOM | 1233 | SG | CYS | A | 191 | 4.992 | 30.845 | 20.193 | 1.00 | 43.49 | S |
| ATOM | 1234 | N | ASP | A | 192 | 1.142 | 28.706 | 20.382 | 1.00 | 25.62 | N |
| ATOM | 1235 | CA | ASP | A | 192 | 0.945 | 27.364 | 20.882 | 1.00 | 26.72 | C |
| ATOM | 1236 | C | ASP | A | 192 | 1.509 | 26.360 | 19.892 | 1.00 | 34.60 | C |
| ATOM | 1237 | O | ASP | A | 192 | 1.397 | 26.516 | 18.668 | 1.00 | 32.55 | O |
| ATOM | 1238 | CB | ASP | A | 192 | −0.539 | 27.118 | 21.156 | 1.00 | 39.57 | C |
| ATOM | 1239 | CG | ASP | A | 192 | −1.162 | 28.217 | 22.038 | 1.00 | 67.23 | C |
| ATOM | 1240 | OD1 | ASP | A | 192 | −0.404 | 28.972 | 22.693 | 1.00 | 59.99 | O |
| ATOM | 1241 | OD2 | ASP | A | 192 | −2.410 | 28.332 | 22.071 | 1.00 | 72.48 | O |
| ATOM | 1242 | N | PHE | A | 193 | 2.149 | 25.338 | 20.427 | 1.00 | 23.97 | N |
| ATOM | 1243 | CA | PHE | A | 193 | 2.825 | 24.378 | 19.585 | 1.00 | 31.53 | C |
| ATOM | 1244 | C | PHE | A | 193 | 1.842 | 23.262 | 19.267 | 1.00 | 30.97 | C |
| ATOM | 1245 | O | PHE | A | 193 | 1.985 | 22.124 | 19.735 | 1.00 | 29.86 | O |
| ATOM | 1246 | CB | PHE | A | 193 | 4.116 | 23.876 | 20.263 | 1.00 | 27.75 | C |
| ATOM | 1247 | CG | PHE | A | 193 | 5.098 | 23.245 | 19.317 | 1.00 | 23.98 | C |
| ATOM | 1248 | CD1 | PHE | A | 193 | 5.236 | 23.711 | 18.013 | 1.00 | 26.99 | C |
| ATOM | 1249 | CD2 | PHE | A | 193 | 5.898 | 22.191 | 19.735 | 1.00 | 23.09 | C |
| ATOM | 1250 | CE1 | PHE | A | 193 | 6.152 | 23.120 | 17.132 | 1.00 | 29.76 | C |
| ATOM | 1251 | CE2 | PHE | A | 193 | 6.812 | 21.600 | 18.868 | 1.00 | 28.56 | C |
| ATOM | 1252 | CZ | PHE | A | 193 | 6.943 | 22.063 | 17.564 | 1.00 | 20.82 | C |
| ATOM | 1253 | N | VAL | A | 194 | 0.826 | 23.625 | 18.484 | 1.00 | 28.33 | N |
| ATOM | 1254 | CA | VAL | A | 194 | −0.201 | 22.700 | 18.009 | 1.00 | 28.69 | C |
| ATOM | 1255 | C | VAL | A | 194 | 0.326 | 21.980 | 16.775 | 1.00 | 28.32 | C |
| ATOM | 1256 | O | VAL | A | 194 | 0.437 | 22.562 | 15.701 | 1.00 | 29.43 | O |
| ATOM | 1257 | CB | VAL | A | 194 | −1.497 | 23.445 | 17.610 | 1.00 | 32.85 | C |
| ATOM | 1258 | CG1 | VAL | A | 194 | −2.687 | 22.506 | 17.676 | 1.00 | 30.79 | C |
| ATOM | 1259 | CG2 | VAL | A | 194 | −1.724 | 24.663 | 18.494 | 1.00 | 31.67 | C |
| ATOM | 1260 | N | THR | A | 195 | 0.661 | 20.710 | 16.918 | 1.00 | 26.32 | N |
| ATOM | 1261 | CA | THR | A | 195 | 1.304 | 20.015 | 15.820 | 1.00 | 32.61 | C |
| ATOM | 1262 | C | THR | A | 195 | 0.447 | 18.854 | 15.365 | 1.00 | 30.73 | C |
| ATOM | 1263 | O | THR | A | 195 | −0.520 | 18.497 | 16.029 | 1.00 | 31.90 | O |
| ATOM | 1264 | CB | THR | A | 195 | 2.707 | 19.521 | 16.218 | 1.00 | 37.42 | C |
| ATOM | 1265 | OG1 | THR | A | 195 | 2.592 | 18.377 | 17.073 | 1.00 | 35.35 | O |
| ATOM | 1266 | CG2 | THR | A | 195 | 3.480 | 20.627 | 16.949 | 1.00 | 29.09 | C |
| ATOM | 1267 | N | ASN | A | 196 | 0.789 | 18.275 | 14.223 | 1.00 | 32.68 | N |
| ATOM | 1268 | CA | ASN | A | 196 | 0.086 | 17.093 | 13.761 | 1.00 | 31.79 | C |
| ATOM | 1269 | C | ASN | A | 196 | 0.601 | 15.854 | 14.511 | 1.00 | 29.85 | C |
| ATOM | 1270 | O | ASN | A | 196 | 1.699 | 15.877 | 15.072 | 1.00 | 28.20 | O |
| ATOM | 1271 | CB | ASN | A | 196 | 0.200 | 16.972 | 12.243 | 1.00 | 30.04 | C |
| ATOM | 1272 | CG | ASN | A | 196 | 1.637 | 16.817 | 11.765 | 1.00 | 26.39 | C |
| ATOM | 1273 | OD1 | ASN | A | 196 | 2.235 | 15.761 | 11.915 | 1.00 | 25.28 | O |
| ATOM | 1274 | ND2 | ASN | A | 196 | 2.177 | 17.859 | 11.157 | 1.00 | 26.00 | N |
| ATOM | 1275 | N | ARG | A | 197 | −0.203 | 14.796 | 14.563 | 1.00 | 26.11 | N |
| ATOM | 1276 | CA | ARG | A | 197 | 0.170 | 13.599 | 15.322 | 1.00 | 24.93 | C |
| ATOM | 1277 | C | ARG | A | 197 | 1.451 | 12.929 | 14.790 | 1.00 | 28.75 | C |
| ATOM | 1278 | O | ARG | A | 197 | 2.294 | 12.471 | 15.575 | 1.00 | 27.62 | O |
| ATOM | 1279 | CB | ARG | A | 197 | −0.986 | 12.584 | 15.394 | 1.00 | 22.15 | C |
| ATOM | 1280 | CG | ARG | A | 197 | −2.313 | 13.143 | 15.883 | 1.00 | 34.94 | C |
| ATOM | 1281 | CD | ARG | A | 197 | −3.333 | 12.028 | 16.099 | 1.00 | 47.76 | C |
| ATOM | 1282 | NE | ARG | A | 197 | −4.677 | 12.521 | 16.418 | 1.00 | 75.52 | N |
| ATOM | 1283 | CZ | ARG | A | 197 | −5.710 | 11.736 | 16.733 | 1.00 | 93.33 | C |
| ATOM | 1284 | NH1 | ARG | A | 197 | −5.561 | 10.416 | 16.781 | 1.00 | 86.60 | N |
| ATOM | 1285 | NH2 | ARG | A | 197 | −6.897 | 12.266 | 17.008 | 1.00 | 91.35 | N |
| ATOM | 1286 | N | ALA | A | 198 | 1.593 | 12.870 | 13.464 | 1.00 | 23.92 | N |
| ATOM | 1287 | CA | ALA | A | 198 | 2.799 | 12.314 | 12.849 | 1.00 | 29.85 | C |
| ATOM | 1288 | C | ALA | A | 198 | 4.050 | 12.976 | 13.422 | 1.00 | 29.84 | C |
| ATOM | 1289 | O | ALA | A | 198 | 4.938 | 12.307 | 13.933 | 1.00 | 27.52 | O |
| ATOM | 1290 | CB | ALA | A | 198 | 2.766 | 12.479 | 11.335 | 1.00 | 24.41 | C |
| ATOM | 1291 | N | TYR | A | 199 | 4.104 | 14.298 | 13.333 | 1.00 | 26.70 | N |
| ATOM | 1292 | CA | TYR | A | 199 | 5.239 | 15.047 | 13.820 | 1.00 | 23.03 | C |
| ATOM | 1293 | C | TYR | A | 199 | 5.431 | 14.850 | 15.317 | 1.00 | 28.75 | C |
| ATOM | 1294 | O | TYR | A | 199 | 6.537 | 14.554 | 15.792 | 1.00 | 28.17 | O |
| ATOM | 1295 | CB | TYR | A | 199 | 5.081 | 16.534 | 13.510 | 1.00 | 23.59 | C |
| ATOM | 1296 | CG | TYR | A | 199 | 6.165 | 17.361 | 14.150 | 1.00 | 26.12 | C |
| ATOM | 1297 | CD1 | TYR | A | 199 | 7.386 | 17.563 | 13.513 | 1.00 | 25.09 | C |
| ATOM | 1298 | CD2 | TYR | A | 199 | 5.984 | 17.906 | 15.413 | 1.00 | 26.43 | C |
| ATOM | 1299 | CE1 | TYR | A | 199 | 8.382 | 18.308 | 14.109 | 1.00 | 23.55 | C |
| ATOM | 1300 | CE2 | TYR | A | 199 | 6.969 | 18.651 | 16.019 | 1.00 | 27.78 | C |
| ATOM | 1301 | CZ | TYR | A | 199 | 8.167 | 18.851 | 15.367 | 1.00 | 23.70 | C |
| ATOM | 1302 | OH | TYR | A | 199 | 9.145 | 19.596 | 15.987 | 1.00 | 21.58 | O |
| ATOM | 1303 | N | ALA | A | 200 | 4.352 | 15.019 | 16.066 | 1.00 | 29.99 | N |
| ATOM | 1304 | CA | ALA | A | 200 | 4.434 | 14.921 | 17.509 | 1.00 | 24.14 | C |
| ATOM | 1305 | C | ALA | A | 200 | 5.115 | 13.617 | 17.940 | 1.00 | 26.61 | C |
| ATOM | 1306 | O | ALA | A | 200 | 6.018 | 13.630 | 18.761 | 1.00 | 29.85 | O |
| ATOM | 1307 | CB | ALA | A | 200 | 3.062 | 15.048 | 18.120 | 1.00 | 24.34 | C |
| ATOM | 1308 | N | ILE | A | 201 | 4.690 | 12.492 | 17.383 | 1.00 | 25.04 | N |
| ATOM | 1309 | CA | ILE | A | 201 | 5.314 | 11.217 | 17.729 | 1.00 | 25.73 | C |
| ATOM | 1310 | C | ILE | A | 201 | 6.763 | 11.089 | 17.234 | 1.00 | 33.55 | C |
| ATOM | 1311 | O | ILE | A | 201 | 7.639 | 10.653 | 17.968 | 1.00 | 31.35 | O |
| ATOM | 1312 | CB | ILE | A | 201 | 4.506 | 10.037 | 17.182 | 1.00 | 34.72 | C |
| ATOM | 1313 | CG1 | ILE | A | 201 | 3.220 | 9.869 | 18.001 | 1.00 | 28.60 | C |
| ATOM | 1314 | CG2 | ILE | A | 201 | 5.369 | 8.757 | 17.161 | 1.00 | 18.19 | C |
| ATOM | 1315 | CD1 | ILE | A | 201 | 2.202 | 8.978 | 17.341 | 1.00 | 28.71 | C |
| ATOM | 1316 | N | ALA | A | 202 | 7.010 | 11.470 | 15.987 | 1.00 | 31.95 | N |
| ATOM | 1317 | CA | ALA | A | 202 | 8.322 | 11.296 | 15.391 | 1.00 | 29.74 | C |
| ATOM | 1318 | C | ALA | A | 202 | 9.380 | 12.173 | 16.053 | 1.00 | 30.66 | C |
| ATOM | 1319 | O | ALA | A | 202 | 10.459 | 11.693 | 16.408 | 1.00 | 28.11 | O |
| ATOM | 1320 | CB | ALA | A | 202 | 8.270 | 11.547 | 13.872 | 1.00 | 26.00 | C |
| ATOM | 1321 | N | SER | A | 203 | 9.078 | 13.455 | 16.223 | 1.00 | 28.64 | N |
| ATOM | 1322 | CA | SER | A | 203 | 10.065 | 14.372 | 16.786 | 1.00 | 30.99 | C |
| ATOM | 1323 | C | SER | A | 203 | 10.384 | 14.005 | 18.234 | 1.00 | 30.08 | C |
| ATOM | 1324 | O | SER | A | 203 | 11.432 | 14.360 | 18.756 | 1.00 | 26.38 | O |
| ATOM | 1325 | CB | SER | A | 203 | 9.584 | 15.815 | 16.699 | 1.00 | 29.18 | C |
| ATOM | 1326 | OG | SER | A | 203 | 8.628 | 16.085 | 17.698 | 1.00 | 26.85 | O |
| ATOM | 1327 | N | SER | A | 204 | 9.470 | 13.280 | 18.864 | 1.00 | 26.34 | N |
| ATOM | 1328 | CA | SER | A | 204 | 9.640 | 12.848 | 20.237 | 1.00 | 27.24 | C |
| ATOM | 1329 | C | SER | A | 204 | 10.467 | 11.569 | 20.339 | 1.00 | 28.35 | C |
| ATOM | 1330 | O | SER | A | 204 | 11.358 | 11.459 | 21.174 | 1.00 | 28.24 | O |
| ATOM | 1331 | CB | SER | A | 204 | 8.274 | 12.653 | 20.883 | 1.00 | 27.86 | C |
| ATOM | 1332 | OG | SER | A | 204 | 7.561 | 13.876 | 20.851 | 1.00 | 31.39 | O |
| ATOM | 1333 | N | ILE | A | 205 | 10.155 | 10.595 | 19.497 | 1.00 | 29.98 | N |
| ATOM | 1334 | CA | ILE | A | 205 | 10.971 | 9.401 | 19.383 | 1.00 | 29.38 | C |
| ATOM | 1335 | C | ILE | A | 205 | 12.412 | 9.795 | 19.063 | 1.00 | 36.57 | C |
| ATOM | 1336 | O | ILE | A | 205 | 13.364 | 9.237 | 19.617 | 1.00 | 34.55 | O |
| ATOM | 1337 | CB | ILE | A | 205 | 10.416 | 8.480 | 18.282 | 1.00 | 37.14 | C |
| ATOM | 1338 | CG1 | ILE | A | 205 | 9.277 | 7.632 | 18.843 | 1.00 | 38.59 | C |
| ATOM | 1339 | CG2 | ILE | A | 205 | 11.499 | 7.575 | 17.701 | 1.00 | 31.15 | C |
| ATOM | 1340 | CD1 | ILE | A | 205 | 8.444 | 6.946 | 17.772 | 1.00 | 32.70 | C |
| ATOM | 1341 | N | ILE | A | 206 | 12.557 | 10.790 | 18.193 | 1.00 | 31.89 | N |
| ATOM | 1342 | CA | ILE | A | 206 | 13.858 | 11.160 | 17.642 | 1.00 | 31.40 | C |
| ATOM | 1343 | C | ILE | A | 206 | 14.680 | 12.156 | 18.468 | 1.00 | 32.64 | C |
| ATOM | 1344 | O | ILE | A | 206 | 15.909 | 12.099 | 18.440 | 1.00 | 37.42 | O |
| ATOM | 1345 | CB | ILE | A | 206 | 13.712 | 11.647 | 16.173 | 1.00 | 32.59 | C |
| ATOM | 1346 | CG1 | ILE | A | 206 | 13.859 | 10.460 | 15.222 | 1.00 | 29.53 | C |
| ATOM | 1347 | CG2 | ILE | A | 206 | 14.721 | 12.747 | 15.838 | 1.00 | 28.85 | C |
| ATOM | 1348 | CD1 | ILE | A | 206 | 12.984 | 10.556 | 14.006 | 1.00 | 34.69 | C |
| ATOM | 1349 | N | SER | A | 207 | 14.020 | 13.063 | 19.188 | 1.00 | 27.31 | N |
| ATOM | 1350 | CA | SER | A | 207 | 14.741 | 14.032 | 20.012 | 1.00 | 25.97 | C |
| ATOM | 1351 | C | SER | A | 207 | 14.973 | 13.501 | 21.409 | 1.00 | 25.69 | C |
| ATOM | 1352 | O | SER | A | 207 | 15.924 | 13.890 | 22.076 | 1.00 | 33.19 | O |
| ATOM | 1353 | CB | SER | A | 207 | 13.991 | 15.363 | 20.110 | 1.00 | 27.32 | C |
| ATOM | 1354 | OG | SER | A | 207 | 13.965 | 16.065 | 18.873 | 1.00 | 30.81 | O |
| ATOM | 1355 | N | PHE | A | 208 | 14.109 | 12.603 | 21.857 | 1.00 | 27.94 | N |
| ATOM | 1356 | CA | PHE | A | 208 | 14.063 | 12.287 | 23.280 | 1.00 | 24.68 | C |
| ATOM | 1357 | C | PHE | A | 208 | 14.166 | 10.806 | 23.635 | 1.00 | 27.29 | C |
| ATOM | 1358 | O | PHE | A | 208 | 15.117 | 10.399 | 24.302 | 1.00 | 36.16 | O |
| ATOM | 1359 | CB | PHE | A | 208 | 12.819 | 12.911 | 23.937 | 1.00 | 24.28 | C |
| ATOM | 1360 | CG | PHE | A | 208 | 12.767 | 12.713 | 25.418 | 1.00 | 26.55 | C |
| ATOM | 1361 | CD1 | PHE | A | 208 | 13.546 | 13.495 | 26.268 | 1.00 | 32.55 | C |
| ATOM | 1362 | CD2 | PHE | A | 208 | 11.987 | 11.713 | 25.966 | 1.00 | 25.08 | C |
| ATOM | 1363 | CE1 | PHE | A | 208 | 13.528 | 13.283 | 27.642 | 1.00 | 20.70 | C |
| ATOM | 1364 | CE2 | PHE | A | 208 | 11.960 | 11.513 | 27.319 | 1.00 | 21.33 | C |
| ATOM | 1365 | CZ | PHE | A | 208 | 12.732 | 12.295 | 28.155 | 1.00 | 20.46 | C |
| ATOM | 1366 | N | TYR | A | 209 | 13.193 | 10.009 | 23.202 | 1.00 | 30.38 | N |
| ATOM | 1367 | CA | TYR | A | 209 | 13.093 | 8.606 | 23.619 | 1.00 | 34.21 | C |
| ATOM | 1368 | C | TYR | A | 209 | 14.255 | 7.709 | 23.177 | 1.00 | 32.23 | C |
| ATOM | 1369 | O | TYR | A | 209 | 14.728 | 6.880 | 23.945 | 1.00 | 34.31 | O |
| ATOM | 1370 | CB | TYR | A | 209 | 11.745 | 7.993 | 23.199 | 1.00 | 31.12 | C |
| ATOM | 1371 | CG | TYR | A | 209 | 10.569 | 8.498 | 24.004 | 1.00 | 32.22 | C |
| ATOM | 1372 | CD1 | TYR | A | 209 | 9.618 | 9.328 | 23.431 | 1.00 | 33.48 | C |
| ATOM | 1373 | CD2 | TYR | A | 209 | 10.417 | 8.156 | 25.344 | 1.00 | 37.94 | C |
| ATOM | 1374 | CE1 | TYR | A | 209 | 8.537 | 9.796 | 24.162 | 1.00 | 31.62 | C |
| ATOM | 1375 | CE2 | TYR | A | 209 | 9.338 | 8.619 | 26.083 | 1.00 | 36.43 | C |
| ATOM | 1376 | CZ | TYR | A | 209 | 8.402 | 9.441 | 25.484 | 1.00 | 39.13 | C |
| ATOM | 1377 | OH | TYR | A | 209 | 7.329 | 9.914 | 26.202 | 1.00 | 43.68 | O |
| ATOM | 1378 | N | ILE | A | 210 | 14.719 | 7.851 | 21.946 | 1.00 | 32.61 | N |
| ATOM | 1379 | CA | ILE | A | 210 | 15.854 | 7.032 | 21.531 | 1.00 | 34.83 | C |
| ATOM | 1380 | C | ILE | A | 210 | 17.142 | 7.413 | 22.261 | 1.00 | 26.35 | C |
| ATOM | 1381 | O | ILE | A | 210 | 17.748 | 6.574 | 22.912 | 1.00 | 31.51 | O |
| ATOM | 1382 | CB | ILE | A | 210 | 16.035 | 6.989 | 20.003 | 1.00 | 33.32 | C |
| ATOM | 1383 | CG1 | ILE | A | 210 | 14.942 | 6.116 | 19.385 | 1.00 | 28.85 | C |
| ATOM | 1384 | CG2 | ILE | A | 210 | 17.406 | 6.433 | 19.634 | 1.00 | 33.18 | C |
| ATOM | 1385 | CD1 | ILE | A | 210 | 14.731 | 6.380 | 17.910 | 1.00 | 32.45 | C |
| ATOM | 1386 | N | PRO | A | 211 | 17.556 | 8.682 | 22.175 | 1.00 | 27.02 | N |
| ATOM | 1387 | CA | PRO | A | 211 | 18.757 | 9.082 | 22.921 | 1.00 | 29.20 | C |
| ATOM | 1388 | C | PRO | A | 211 | 18.639 | 8.675 | 24.390 | 1.00 | 38.43 | C |
| ATOM | 1389 | O | PRO | A | 211 | 19.628 | 8.268 | 24.993 | 1.00 | 40.54 | O |
| ATOM | 1390 | CB | PRO | A | 211 | 18.755 | 10.610 | 22.813 | 1.00 | 27.67 | C |
| ATOM | 1391 | CG | PRO | A | 211 | 17.932 | 10.912 | 21.620 | 1.00 | 35.57 | C |
| ATOM | 1392 | CD | PRO | A | 211 | 16.933 | 9.805 | 21.459 | 1.00 | 26.23 | C |
| ATOM | 1393 | N | LEU | A | 212 | 17.436 | 8.777 | 24.954 | 1.00 | 33.08 | N |
| ATOM | 1394 | CA | LEU | A | 212 | 17.196 | 8.355 | 26.332 | 1.00 | 29.10 | C |
| ATOM | 1395 | C | LEU | A | 212 | 17.441 | 6.860 | 26.560 | 1.00 | 33.70 | C |
| ATOM | 1396 | O | LEU | A | 212 | 18.214 | 6.481 | 27.437 | 1.00 | 38.29 | O |
| ATOM | 1397 | CB | LEU | A | 212 | 15.779 | 8.717 | 26.772 | 1.00 | 30.14 | C |
| ATOM | 1398 | CG | LEU | A | 212 | 15.534 | 8.587 | 28.279 | 1.00 | 31.90 | C |
| ATOM | 1399 | CD1 | LEU | A | 212 | 16.223 | 9.720 | 29.057 | 1.00 | 21.99 | C |
| ATOM | 1400 | CD2 | LEU | A | 212 | 14.060 | 8.573 | 28.556 | 1.00 | 26.71 | C |
| ATOM | 1401 | N | LEU | A | 213 | 16.776 | 6.020 | 25.775 | 1.00 | 34.42 | N |
| ATOM | 1402 | CA | LEU | A | 213 | 16.964 | 4.576 | 25.849 | 1.00 | 33.11 | C |
| ATOM | 1403 | C | LEU | A | 213 | 18.431 | 4.178 | 25.718 | 1.00 | 34.57 | C |
| ATOM | 1404 | O | LEU | A | 213 | 18.883 | 3.244 | 26.371 | 1.00 | 29.68 | O |
| ATOM | 1405 | CB | LEU | A | 213 | 16.140 | 3.881 | 24.768 | 1.00 | 32.56 | C |
| ATOM | 1406 | CG | LEU | A | 213 | 14.635 | 4.014 | 24.996 | 1.00 | 48.49 | C |
| ATOM | 1407 | CD1 | LEU | A | 213 | 13.857 | 3.555 | 23.778 | 1.00 | 45.04 | C |
| ATOM | 1408 | CD2 | LEU | A | 213 | 14.206 | 3.240 | 26.235 | 1.00 | 40.44 | C |
| ATOM | 1409 | N | ILE | A | 214 | 19.169 | 4.889 | 24.867 | 1.00 | 31.91 | N |
| ATOM | 1410 | CA | ILE | A | 214 | 20.591 | 4.628 | 24.694 | 1.00 | 31.18 | C |
| ATOM | 1411 | C | ILE | A | 214 | 21.356 | 5.016 | 25.943 | 1.00 | 38.97 | C |
| ATOM | 1412 | O | ILE | A | 214 | 22.099 | 4.212 | 26.507 | 1.00 | 41.86 | O |
| ATOM | 1413 | CB | ILE | A | 214 | 21.180 | 5.426 | 23.529 | 1.00 | 35.18 | C |
| ATOM | 1414 | CG1 | ILE | A | 214 | 20.702 | 4.856 | 22.204 | 1.00 | 31.13 | C |
| ATOM | 1415 | CG2 | ILE | A | 214 | 22.714 | 5.419 | 23.584 | 1.00 | 30.25 | C |
| ATOM | 1416 | CD1 | ILE | A | 214 | 21.109 | 5.689 | 21.046 | 1.00 | 32.06 | C |
| ATOM | 1417 | N | MET | A | 215 | 21.171 | 6.258 | 26.374 | 1.00 | 31.21 | N |
| ATOM | 1418 | CA | MET | A | 215 | 21.867 | 6.764 | 27.546 | 1.00 | 34.92 | C |
| ATOM | 1419 | C | MET | A | 215 | 21.683 | 5.873 | 28.778 | 1.00 | 40.82 | C |
| ATOM | 1420 | O | MET | A | 215 | 22.620 | 5.676 | 29.545 | 1.00 | 36.54 | O |
| ATOM | 1421 | CB | MET | A | 215 | 21.417 | 8.184 | 27.867 | 1.00 | 35.42 | C |
| ATOM | 1422 | CG | MET | A | 215 | 22.183 | 8.813 | 29.015 | 1.00 | 33.32 | C |
| ATOM | 1423 | SD | MET | A | 215 | 21.246 | 10.158 | 29.733 | 1.00 | 48.20 | S |
| ATOM | 1424 | CE | MET | A | 215 | 19.950 | 9.220 | 30.541 | 1.00 | 39.49 | C |
| ATOM | 1425 | N | ILE | A | 216 | 20.475 | 5.346 | 28.965 | 1.00 | 36.27 | N |
| ATOM | 1426 | CA | ILE | A | 216 | 20.196 | 4.476 | 30.094 | 1.00 | 36.52 | C |
| ATOM | 1427 | C | ILE | A | 216 | 20.876 | 3.127 | 29.945 | 1.00 | 40.79 | C |
| ATOM | 1428 | O | ILE | A | 216 | 21.622 | 2.707 | 30.828 | 1.00 | 45.81 | O |
| ATOM | 1429 | CB | ILE | A | 216 | 18.689 | 4.261 | 30.313 | 1.00 | 45.28 | C |
| ATOM | 1430 | CG1 | ILE | A | 216 | 18.121 | 5.400 | 31.160 | 1.00 | 39.15 | C |
| ATOM | 1431 | CG2 | ILE | A | 216 | 18.432 | 2.908 | 30.991 | 1.00 | 26.27 | C |
| ATOM | 1432 | CD1 | ILE | A | 216 | 16.675 | 5.723 | 30.841 | 1.00 | 35.73 | C |
| ATOM | 1433 | N | PHE | A | 217 | 20.620 | 2.443 | 28.839 | 1.00 | 31.07 | N |
| ATOM | 1434 | CA | PHE | A | 217 | 21.259 | 1.164 | 28.615 | 1.00 | 32.64 | C |
| ATOM | 1435 | C | PHE | A | 217 | 22.775 | 1.272 | 28.782 | 1.00 | 44.70 | C |
| ATOM | 1436 | O | PHE | A | 217 | 23.409 | 0.369 | 29.322 | 1.00 | 46.59 | O |
| ATOM | 1437 | CB | PHE | A | 217 | 20.921 | 0.605 | 27.239 | 1.00 | 42.52 | C |
| ATOM | 1438 | CG | PHE | A | 217 | 21.672 | −0.645 | 26.908 | 1.00 | 59.40 | C |
| ATOM | 1439 | CD1 | PHE | A | 217 | 21.379 | −1.832 | 27.557 | 1.00 | 66.77 | C |
| ATOM | 1440 | CD2 | PHE | A | 217 | 22.691 | −0.633 | 25.965 | 1.00 | 73.28 | C |
| ATOM | 1441 | CE1 | PHE | A | 217 | 22.079 | −2.993 | 27.264 | 1.00 | 79.21 | C |
| ATOM | 1442 | CE2 | PHE | A | 217 | 23.397 | −1.792 | 25.661 | 1.00 | 68.73 | C |
| ATOM | 1443 | CZ | PHE | A | 217 | 23.090 | −2.972 | 26.313 | 1.00 | 75.53 | C |
| ATOM | 1444 | N | VAL | A | 218 | 23.357 | 2.380 | 28.334 | 1.00 | 42.28 | N |
| ATOM | 1445 | CA | VAL | A | 218 | 24.801 | 2.574 | 28.456 | 1.00 | 41.92 | C |
| ATOM | 1446 | C | VAL | A | 218 | 25.236 | 2.913 | 29.888 | 1.00 | 40.46 | C |
| ATOM | 1447 | O | VAL | A | 218 | 26.172 | 2.316 | 30.415 | 1.00 | 39.77 | O |
| ATOM | 1448 | CB | VAL | A | 218 | 25.331 | 3.630 | 27.452 | 1.00 | 43.63 | C |
| ATOM | 1449 | CG1 | VAL | A | 218 | 26.761 | 4.039 | 27.784 | 1.00 | 33.36 | C |
| ATOM | 1450 | CG2 | VAL | A | 218 | 25.258 | 3.085 | 26.035 | 1.00 | 30.46 | C |
| ATOM | 1451 | N | ALA | A | 219 | 24.554 | 3.863 | 30.515 | 1.00 | 42.47 | N |
| ATOM | 1452 | CA | ALA | A | 219 | 24.829 | 4.206 | 31.905 | 1.00 | 39.30 | C |
| ATOM | 1453 | C | ALA | A | 219 | 24.733 | 2.994 | 32.828 | 1.00 | 40.43 | C |
| ATOM | 1454 | O | ALA | A | 219 | 25.494 | 2.884 | 33.786 | 1.00 | 42.68 | O |
| ATOM | 1455 | CB | ALA | A | 219 | 23.891 | 5.303 | 32.379 | 1.00 | 35.20 | C |
| ATOM | 1456 | N | LEU | A | 220 | 23.800 | 2.090 | 32.547 | 1.00 | 37.44 | N |
| ATOM | 1457 | CA | LEU | A | 220 | 23.673 | 0.880 | 33.347 | 1.00 | 42.28 | C |
| ATOM | 1458 | C | LEU | A | 220 | 24.886 | −0.045 | 33.176 | 1.00 | 46.75 | C |
| ATOM | 1459 | O | LEU | A | 220 | 25.362 | −0.637 | 34.148 | 1.00 | 54.13 | O |
| ATOM | 1460 | CB | LEU | A | 220 | 22.358 | 0.147 | 33.049 | 1.00 | 37.79 | C |
| ATOM | 1461 | CG | LEU | A | 220 | 21.096 | 0.829 | 33.605 | 1.00 | 56.15 | C |
| ATOM | 1462 | CD1 | LEU | A | 220 | 19.930 | −0.151 | 33.701 | 1.00 | 42.10 | C |
| ATOM | 1463 | CD2 | LEU | A | 220 | 21.334 | 1.514 | 34.965 | 1.00 | 23.99 | C |
| ATOM | 1464 | N | ARG | A | 221 | 25.375 | −0.162 | 31.940 | 1.00 | 34.06 | N |
| ATOM | 1465 | CA | ARG | A | 221 | 26.633 | −0.857 | 31.645 | 1.00 | 47.01 | C |
| ATOM | 1466 | C | ARG | A | 221 | 27.847 | −0.270 | 32.381 | 1.00 | 44.19 | C |
| ATOM | 1467 | O | ARG | A | 221 | 28.697 | −1.004 | 32.875 | 1.00 | 49.15 | O |
| ATOM | 1468 | CB | ARG | A | 221 | 26.922 | −0.852 | 30.135 | 1.00 | 51.35 | C |
| ATOM | 1469 | CG | ARG | A | 221 | 26.069 | −1.807 | 29.295 | 1.00 | 47.59 | C |
| ATOM | 1470 | CD | ARG | A | 221 | 25.852 | −3.157 | 29.978 | 1.00 | 60.46 | C |
| ATOM | 1471 | NE | ARG | A | 221 | 27.105 | −3.818 | 30.337 | 1.00 | 81.42 | N |
| ATOM | 1472 | CZ | ARG | A | 221 | 27.181 | −4.943 | 31.044 | 1.00 | 86.87 | C |
| ATOM | 1473 | NH1 | ARG | A | 221 | 26.071 | −5.536 | 31.465 | 1.00 | 84.36 | N |
| ATOM | 1474 | NH2 | ARG | A | 221 | 28.366 | −5.475 | 31.329 | 1.00 | 94.79 | N |
| ATOM | 1475 | N | VAL | A | 222 | 27.926 | 1.056 | 32.431 | 1.00 | 50.12 | N |
| ATOM | 1476 | CA | VAL | A | 222 | 28.997 | 1.765 | 33.130 | 1.00 | 46.09 | C |
| ATOM | 1477 | C | VAL | A | 222 | 28.925 | 1.534 | 34.639 | 1.00 | 53.14 | C |
| ATOM | 1478 | O | VAL | A | 222 | 29.954 | 1.329 | 35.288 | 1.00 | 53.44 | O |
| ATOM | 1479 | CB | VAL | A | 222 | 28.958 | 3.282 | 32.818 | 1.00 | 35.48 | C |
| ATOM | 1480 | CG1 | VAL | A | 222 | 29.921 | 4.055 | 33.704 | 1.00 | 33.70 | C |
| ATOM | 1481 | CG2 | VAL | A | 222 | 29.276 | 3.522 | 31.364 | 1.00 | 37.85 | C |
| ATOM | 1482 | N | TYR | A | 223 | 27.709 | 1.566 | 35.187 | 1.00 | 47.31 | N |
| ATOM | 1483 | CA | TYR | A | 223 | 27.472 | 1.227 | 36.591 | 1.00 | 56.01 | C |
| ATOM | 1484 | C | TYR | A | 223 | 27.973 | −0.187 | 36.919 | 1.00 | 61.05 | C |
| ATOM | 1485 | O | TYR | A | 223 | 28.700 | −0.388 | 37.895 | 1.00 | 58.67 | O |
| ATOM | 1486 | CB | TYR | A | 223 | 25.980 | 1.342 | 36.915 | 1.00 | 55.83 | C |
| ATOM | 1487 | CG | TYR | A | 223 | 25.614 | 1.018 | 38.353 | 1.00 | 76.27 | C |
| ATOM | 1488 | CD1 | TYR | A | 223 | 25.703 | 1.986 | 39.348 | 1.00 | 78.06 | C |
| ATOM | 1489 | CD2 | TYR | A | 223 | 25.168 | −0.251 | 38.711 | 1.00 | 68.54 | C |
| ATOM | 1490 | CE1 | TYR | A | 223 | 25.367 | 1.700 | 40.656 | 1.00 | 81.59 | C |
| ATOM | 1491 | CE2 | TYR | A | 223 | 24.834 | −0.548 | 40.019 | 1.00 | 67.78 | C |
| ATOM | 1492 | CZ | TYR | A | 223 | 24.934 | 0.432 | 40.987 | 1.00 | 89.61 | C |
| ATOM | 1493 | OH | TYR | A | 223 | 24.600 | 0.148 | 42.292 | 1.00 | 103.52 | O |
| ATOM | 1494 | N | ARG | A | 224 | 27.586 | −1.144 | 36.074 | 1.00 | 47.11 | N |
| ATOM | 1495 | CA | ARG | A | 224 | 27.962 | −2.554 | 36.188 | 1.00 | 52.64 | C |
| ATOM | 1496 | C | ARG | A | 224 | 29.468 | −2.783 | 36.074 | 1.00 | 58.78 | C |
| ATOM | 1497 | O | ARG | A | 224 | 29.998 | −3.746 | 36.604 | 1.00 | 61.77 | O |
| ATOM | 1498 | CB | ARG | A | 224 | 27.249 | −3.357 | 35.096 | 1.00 | 53.18 | C |
| ATOM | 1499 | CG | ARG | A | 224 | 27.122 | −4.847 | 35.365 | 1.00 | 67.01 | C |
| ATOM | 1500 | CD | ARG | A | 224 | 28.441 | −5.575 | 35.182 | 1.00 | 73.85 | C |
| ATOM | 1501 | NE | ARG | A | 224 | 28.354 | −6.976 | 35.590 | 1.00 | 92.74 | N |
| ATOM | 1502 | CZ | ARG | A | 224 | 28.427 | −7.403 | 36.849 | 1.00 | 89.22 | C |
| ATOM | 1503 | NH1 | ARG | A | 224 | 28.595 | −6.535 | 37.836 | 1.00 | 99.73 | N |
| ATOM | 1504 | NH2 | ARG | A | 224 | 28.334 | −8.700 | 37.125 | 1.00 | 81.42 | N |
| ATOM | 1505 | N | GLU | A | 225 | 30.149 | −1.907 | 35.356 | 1.00 | 62.29 | N |
| ATOM | 1506 | CA | GLU | A | 225 | 31.599 | −1.961 | 35.232 | 1.00 | 61.05 | C |
| ATOM | 1507 | C | GLU | A | 225 | 32.295 | −1.296 | 36.416 | 1.00 | 67.83 | C |
| ATOM | 1508 | O | GLU | A | 225 | 33.271 | −1.822 | 36.938 | 1.00 | 78.23 | O |
| ATOM | 1509 | CB | GLU | A | 225 | 32.044 | −1.305 | 33.923 | 1.00 | 59.05 | C |
| ATOM | 1510 | CG | GLU | A | 225 | 32.030 | −2.247 | 32.723 | 1.00 | 75.98 | C |
| ATOM | 1511 | CD | GLU | A | 225 | 33.138 | −3.290 | 32.779 | 1.00 | 84.71 | C |
| ATOM | 1512 | OE1 | GLU | A | 225 | 33.760 | −3.454 | 33.850 | 1.00 | 86.02 | O |
| ATOM | 1513 | OE2 | GLU | A | 225 | 33.389 | −3.944 | 31.746 | 1.00 | 89.53 | O |
| ATOM | 1514 | N | ALA | A | 226 | 31.790 | −0.140 | 36.836 | 1.00 | 61.93 | N |
| ATOM | 1515 | CA | ALA | A | 226 | 32.343 | 0.561 | 37.986 | 1.00 | 62.44 | C |
| ATOM | 1516 | C | ALA | A | 226 | 32.159 | −0.269 | 39.250 | 1.00 | 75.28 | C |
| ATOM | 1517 | O | ALA | A | 226 | 32.954 | −0.178 | 40.181 | 1.00 | 86.17 | O |
| ATOM | 1518 | CB | ALA | A | 226 | 31.690 | 1.927 | 38.145 | 1.00 | 54.77 | C |
| ATOM | 1519 | N | LYS | A | 227 | 31.111 | −1.084 | 39.273 | 1.00 | 70.13 | N |
| ATOM | 1520 | CA | LYS | A | 227 | 30.803 | −1.915 | 40.432 | 1.00 | 72.99 | C |
| ATOM | 1521 | C | LYS | A | 227 | 31.701 | −3.143 | 40.486 | 1.00 | 69.03 | C |
| ATOM | 1522 | O | LYS | A | 227 | 32.511 | −3.293 | 41.402 | 1.00 | 79.39 | O |
| ATOM | 1523 | CB | LYS | A | 227 | 29.339 | −2.355 | 40.390 | 1.00 | 86.73 | C |
| ATOM | 1524 | CG | LYS | A | 227 | 28.929 | −3.270 | 41.531 | 1.00 | 94.76 | C |
| ATOM | 1525 | CD | LYS | A | 227 | 27.717 | −4.110 | 41.156 | 1.00 | 88.99 | C |
| ATOM | 1526 | CE | LYS | A | 227 | 27.297 | −5.013 | 42.307 | 1.00 | 116.52 | C |
| ATOM | 1527 | NZ | LYS | A | 227 | 28.421 | −5.862 | 42.804 | 1.00 | 112.93 | N |
| ATOM | 1528 | N | GLU | A | 228 | 31.524 | −4.001 | 39.479 | 1.00 | 64.08 | N |
| ATOM | 1529 | CA | GLU | A | 228 | 32.320 | −5.206 | 39.202 | 1.00 | 74.72 | C |
| ATOM | 1530 | C | GLU | A | 228 | 33.802 | −4.920 | 39.309 | 1.00 | 76.43 | C |
| ATOM | 1531 | O | GLU | A | 228 | 34.637 | −5.814 | 39.219 | 1.00 | 70.99 | O |
| ATOM | 1532 | CB | GLU | A | 228 | 32.022 | −5.674 | 37.771 | 1.00 | 71.15 | C |
| ATOM | 1533 | CG | GLU | A | 228 | 32.402 | −7.103 | 37.426 | 1.00 | 63.61 | C |
| ATOM | 1534 | CD | GLU | A | 228 | 32.020 | −7.477 | 35.983 | 1.00 | 104.77 | C |
| ATOM | 1535 | OE1 | GLU | A | 228 | 32.297 | −6.678 | 35.056 | 1.00 | 84.00 | O |
| ATOM | 1536 | OE2 | GLU | A | 228 | 31.442 | −8.572 | 35.776 | 1.00 | 107.16 | O |
| ATOM | 1537 | N | GLN | A | 229 | 34.108 | −3.648 | 39.501 | 1.00 | 83.37 | N |
| ATOM | 1538 | CA | GLN | A | 229 | 35.464 | −3.142 | 39.518 | 1.00 | 80.99 | C |
| ATOM | 1539 | C | GLN | A | 229 | 35.900 | −2.898 | 40.953 | 1.00 | 90.06 | C |
| ATOM | 1540 | O | GLN | A | 229 | 37.006 | −3.260 | 41.348 | 1.00 | 104.26 | O |
| ATOM | 1541 | CB | GLN | A | 229 | 35.478 | −1.815 | 38.775 | 1.00 | 84.09 | C |
| ATOM | 1542 | CG | GLN | A | 229 | 36.733 | −1.509 | 38.015 | 1.00 | 76.29 | C |
| ATOM | 1543 | CD | GLN | A | 229 | 36.716 | −0.099 | 37.472 | 1.00 | 79.45 | C |
| ATOM | 1544 | OE1 | GLN | A | 229 | 37.218 | 0.163 | 36.379 | 1.00 | 80.07 | O |
| ATOM | 1545 | NE2 | GLN | A | 229 | 36.122 | 0.822 | 38.230 | 1.00 | 83.76 | N |
| ATOM | 1546 | N | ILE | A | 230 | 35.011 | −2.271 | 41.721 | 1.00 | 94.97 | N |
| ATOM | 1547 | CA | ILE | A | 230 | 35.277 | −1.834 | 43.087 | 1.00 | 91.78 | C |
| ATOM | 1548 | C | ILE | A | 230 | 34.317 | −0.697 | 43.378 | 1.00 | 77.23 | C |
| ATOM | 1549 | O | ILE | A | 230 | 33.122 | −0.909 | 43.540 | 1.00 | 88.60 | O |
| ATOM | 1550 | CB | ILE | A | 230 | 36.710 | −1.294 | 43.254 | 1.00 | 103.06 | C |
| ATOM | 1551 | CG1 | ILE | A | 230 | 36.923 | −0.751 | 44.672 | 1.00 | 103.08 | C |
| ATOM | 1552 | CG2 | ILE | A | 230 | 36.993 | −0.198 | 42.227 | 1.00 | 94.49 | C |
| ATOM | 1553 | CD1 | ILE | A | 230 | 36.454 | 0.687 | 44.868 | 1.00 | 85.77 | C |
| ATOM | 1554 | N | ARG | A | 267 | 38.234 | 7.999 | 43.858 | 1.00 | 98.55 | N |
| ATOM | 1555 | CA | ARG | A | 267 | 37.413 | 9.201 | 43.878 | 1.00 | 102.83 | C |
| ATOM | 1556 | C | ARG | A | 267 | 36.747 | 9.309 | 42.517 | 1.00 | 96.59 | C |
| ATOM | 1557 | O | ARG | A | 267 | 35.927 | 10.196 | 42.272 | 1.00 | 87.82 | O |
| ATOM | 1558 | CB | ARG | A | 267 | 38.268 | 10.441 | 44.138 | 1.00 | 111.38 | C |
| ATOM | 1559 | CG | ARG | A | 267 | 38.820 | 10.543 | 45.551 | 1.00 | 124.33 | C |
| ATOM | 1560 | CD | ARG | A | 267 | 37.745 | 10.951 | 46.554 | 1.00 | 126.95 | C |
| ATOM | 1561 | NE | ARG | A | 267 | 38.293 | 11.114 | 47.900 | 1.00 | 138.98 | N |
| ATOM | 1562 | CZ | ARG | A | 267 | 37.607 | 11.567 | 48.946 | 1.00 | 131.72 | C |
| ATOM | 1563 | NH1 | ARG | A | 267 | 36.332 | 11.909 | 48.814 | 1.00 | 114.09 | N |
| ATOM | 1564 | NH2 | ARG | A | 267 | 38.199 | 11.679 | 50.127 | 1.00 | 134.30 | N |
| ATOM | 1565 | N | GLU | A | 268 | 37.125 | 8.390 | 41.635 | 1.00 | 93.11 | N |
| ATOM | 1566 | CA | GLU | A | 268 | 36.558 | 8.301 | 40.297 | 1.00 | 77.06 | C |
| ATOM | 1567 | C | GLU | A | 268 | 35.093 | 7.881 | 40.351 | 1.00 | 74.27 | C |
| ATOM | 1568 | O | GLU | A | 268 | 34.281 | 8.325 | 39.544 | 1.00 | 69.69 | O |
| ATOM | 1569 | CB | GLU | A | 268 | 37.352 | 7.301 | 39.461 | 1.00 | 92.14 | C |
| ATOM | 1570 | CG | GLU | A | 268 | 38.856 | 7.545 | 39.463 | 1.00 | 122.45 | C |
| ATOM | 1571 | CD | GLU | A | 268 | 39.259 | 8.762 | 38.645 | 1.00 | 134.93 | C |
| ATOM | 1572 | OE1 | GLU | A | 268 | 38.446 | 9.216 | 37.809 | 1.00 | 132.98 | O |
| ATOM | 1573 | OE2 | GLU | A | 268 | 40.391 | 9.259 | 38.835 | 1.00 | 135.31 | O |
| ATOM | 1574 | N | HIS | A | 269 | 34.758 | 7.022 | 41.307 | 1.00 | 80.89 | N |
| ATOM | 1575 | CA | HIS | A | 269 | 33.372 | 6.602 | 41.503 | 1.00 | 69.65 | C |
| ATOM | 1576 | C | HIS | A | 269 | 32.517 | 7.752 | 42.013 | 1.00 | 58.87 | C |
| ATOM | 1577 | O | HIS | A | 269 | 31.298 | 7.741 | 41.869 | 1.00 | 64.21 | O |
| ATOM | 1578 | CB | HIS | A | 269 | 33.297 | 5.407 | 42.454 | 1.00 | 64.13 | C |
| ATOM | 1579 | CG | HIS | A | 269 | 33.790 | 4.128 | 41.845 | 1.00 | 85.61 | C |
| ATOM | 1580 | ND1 | HIS | A | 269 | 33.376 | 2.892 | 42.276 | 1.00 | 87.41 | N |
| ATOM | 1581 | CD2 | HIS | A | 269 | 34.648 | 3.911 | 40.818 | 1.00 | 91.53 | C |
| ATOM | 1582 | CE1 | HIS | A | 269 | 33.974 | 1.955 | 41.550 | 1.00 | 84.25 | C |
| ATOM | 1583 | NE2 | HIS | A | 269 | 34.743 | 2.547 | 40.659 | 1.00 | 82.20 | N |
| ATOM | 1584 | N | LYS | A | 270 | 33.161 | 8.755 | 42.594 | 1.00 | 58.96 | N |
| ATOM | 1585 | CA | LYS | A | 270 | 32.440 | 9.928 | 43.065 | 1.00 | 72.45 | C |
| ATOM | 1586 | C | LYS | A | 270 | 32.155 | 10.879 | 41.904 | 1.00 | 71.67 | C |
| ATOM | 1587 | O | LYS | A | 270 | 31.130 | 11.563 | 41.885 | 1.00 | 67.37 | O |
| ATOM | 1588 | CB | LYS | A | 270 | 33.214 | 10.607 | 44.195 | 1.00 | 71.70 | C |
| ATOM | 1589 | CG | LYS | A | 270 | 33.427 | 9.669 | 45.379 | 1.00 | 98.25 | C |
| ATOM | 1590 | CD | LYS | A | 270 | 34.450 | 10.186 | 46.379 | 1.00 | 125.30 | C |
| ATOM | 1591 | CE | LYS | A | 270 | 34.709 | 9.159 | 47.481 | 1.00 | 120.65 | C |
| ATOM | 1592 | NZ | LYS | A | 270 | 33.469 | 8.790 | 48.224 | 1.00 | 123.08 | N |
| ATOM | 1593 | N | ALA | A | 271 | 33.057 | 10.899 | 40.928 | 1.00 | 59.98 | N |
| ATOM | 1594 | CA | ALA | A | 271 | 32.836 | 11.650 | 39.705 | 1.00 | 48.84 | C |
| ATOM | 1595 | C | ALA | A | 271 | 31.776 | 10.957 | 38.847 | 1.00 | 54.12 | C |
| ATOM | 1596 | O | ALA | A | 271 | 30.956 | 11.612 | 38.197 | 1.00 | 48.88 | O |
| ATOM | 1597 | CB | ALA | A | 271 | 34.127 | 11.795 | 38.943 | 1.00 | 49.40 | C |
| ATOM | 1598 | N | LEU | A | 272 | 31.797 | 9.628 | 38.853 | 1.00 | 44.38 | N |
| ATOM | 1599 | CA | LEU | A | 272 | 30.792 | 8.838 | 38.148 | 1.00 | 47.82 | C |
| ATOM | 1600 | C | LEU | A | 272 | 29.418 | 8.934 | 38.803 | 1.00 | 51.17 | C |
| ATOM | 1601 | O | LEU | A | 272 | 28.393 | 8.820 | 38.132 | 1.00 | 44.10 | O |
| ATOM | 1602 | CB | LEU | A | 272 | 31.208 | 7.368 | 38.070 | 1.00 | 48.29 | C |
| ATOM | 1603 | CG | LEU | A | 272 | 32.341 | 7.038 | 37.102 | 1.00 | 45.28 | C |
| ATOM | 1604 | CD1 | LEU | A | 272 | 32.517 | 5.544 | 37.033 | 1.00 | 41.86 | C |
| ATOM | 1605 | CD2 | LEU | A | 272 | 32.063 | 7.608 | 35.723 | 1.00 | 31.56 | C |
| ATOM | 1606 | N | LYS | A | 273 | 29.388 | 9.128 | 40.115 | 1.00 | 56.57 | N |
| ATOM | 1607 | CA | LYS | A | 273 | 28.107 | 9.180 | 40.799 | 1.00 | 53.05 | C |
| ATOM | 1608 | C | LYS | A | 273 | 27.394 | 10.489 | 40.469 | 1.00 | 49.59 | C |
| ATOM | 1609 | O | LYS | A | 273 | 26.170 | 10.551 | 40.432 | 1.00 | 42.94 | O |
| ATOM | 1610 | CB | LYS | A | 273 | 28.273 | 9.016 | 42.308 | 1.00 | 57.78 | C |
| ATOM | 1611 | CG | LYS | A | 273 | 27.117 | 8.254 | 42.946 | 1.00 | 77.19 | C |
| ATOM | 1612 | CD | LYS | A | 273 | 26.644 | 8.886 | 44.253 | 1.00 | 78.11 | C |
| ATOM | 1613 | CE | LYS | A | 273 | 25.549 | 8.044 | 44.883 | 1.00 | 68.84 | C |
| ATOM | 1614 | NZ | LYS | A | 273 | 24.992 | 8.671 | 46.102 | 1.00 | 86.94 | N |
| ATOM | 1615 | N | THR | A | 274 | 28.177 | 11.531 | 40.222 | 1.00 | 50.27 | N |
| ATOM | 1616 | CA | THR | A | 274 | 27.635 | 12.822 | 39.838 | 1.00 | 45.95 | C |
| ATOM | 1617 | C | THR | A | 274 | 27.096 | 12.772 | 38.408 | 1.00 | 41.97 | C |
| ATOM | 1618 | O | THR | A | 274 | 26.033 | 13.322 | 38.116 | 1.00 | 37.18 | O |
| ATOM | 1619 | CB | THR | A | 274 | 28.700 | 13.922 | 39.965 | 1.00 | 35.46 | C |
| ATOM | 1620 | OG1 | THR | A | 274 | 29.238 | 13.902 | 41.287 | 1.00 | 50.74 | O |
| ATOM | 1621 | CG2 | THR | A | 274 | 28.097 | 15.291 | 39.703 | 1.00 | 41.95 | C |
| ATOM | 1622 | N | LEU | A | 275 | 27.834 | 12.114 | 37.520 | 1.00 | 37.39 | N |
| ATOM | 1623 | CA | LEU | A | 275 | 27.377 | 11.932 | 36.151 | 1.00 | 36.72 | C |
| ATOM | 1624 | C | LEU | A | 275 | 26.046 | 11.190 | 36.155 | 1.00 | 37.97 | C |
| ATOM | 1625 | O | LEU | A | 275 | 25.127 | 11.542 | 35.417 | 1.00 | 28.54 | O |
| ATOM | 1626 | CB | LEU | A | 275 | 28.410 | 11.176 | 35.316 | 1.00 | 28.78 | C |
| ATOM | 1627 | CG | LEU | A | 275 | 29.706 | 11.951 | 35.073 | 1.00 | 42.27 | C |
| ATOM | 1628 | CD1 | LEU | A | 275 | 30.639 | 11.206 | 34.109 | 1.00 | 42.12 | C |
| ATOM | 1629 | CD2 | LEU | A | 275 | 29.411 | 13.359 | 34.555 | 1.00 | 33.72 | C |
| ATOM | 1630 | N | GLY | A | 276 | 25.953 | 10.172 | 37.004 | 1.00 | 38.06 | N |
| ATOM | 1631 | CA | GLY | A | 276 | 24.721 | 9.433 | 37.193 | 1.00 | 35.97 | C |
| ATOM | 1632 | C | GLY | A | 276 | 23.586 | 10.296 | 37.728 | 1.00 | 37.74 | C |
| ATOM | 1633 | O | GLY | A | 276 | 22.452 | 10.179 | 37.275 | 1.00 | 35.84 | O |
| ATOM | 1634 | N | ILE | A | 277 | 23.884 | 11.170 | 38.685 | 1.00 | 37.08 | N |
| ATOM | 1635 | CA | ILE | A | 277 | 22.875 | 12.078 | 39.227 | 1.00 | 32.35 | C |
| ATOM | 1636 | C | ILE | A | 277 | 22.378 | 13.051 | 38.160 | 1.00 | 31.83 | C |
| ATOM | 1637 | O | ILE | A | 277 | 21.185 | 13.302 | 38.059 | 1.00 | 32.48 | O |
| ATOM | 1638 | CB | ILE | A | 277 | 23.406 | 12.852 | 40.462 | 1.00 | 38.79 | C |
| ATOM | 1639 | CG1 | ILE | A | 277 | 23.544 | 11.910 | 41.660 | 1.00 | 28.85 | C |
| ATOM | 1640 | CG2 | ILE | A | 277 | 22.498 | 14.025 | 40.826 | 1.00 | 22.70 | C |
| ATOM | 1641 | CD1 | ILE | A | 277 | 24.625 | 12.318 | 42.636 | 1.00 | 46.73 | C |
| ATOM | 1642 | N | ILE | A | 278 | 23.296 | 13.583 | 37.358 | 1.00 | 36.24 | N |
| ATOM | 1643 | CA | ILE | A | 278 | 22.942 | 14.483 | 36.258 | 1.00 | 35.99 | C |
| ATOM | 1644 | C | ILE | A | 278 | 21.962 | 13.837 | 35.265 | 1.00 | 39.36 | C |
| ATOM | 1645 | O | ILE | A | 278 | 20.994 | 14.465 | 34.830 | 1.00 | 30.79 | O |
| ATOM | 1646 | CB | ILE | A | 278 | 24.206 | 14.948 | 35.513 | 1.00 | 36.39 | C |
| ATOM | 1647 | CG1 | ILE | A | 278 | 25.039 | 15.846 | 36.419 | 1.00 | 51.94 | C |
| ATOM | 1648 | CG2 | ILE | A | 278 | 23.850 | 15.738 | 34.290 | 1.00 | 36.91 | C |
| ATOM | 1649 | CD1 | ILE | A | 278 | 24.463 | 17.221 | 36.562 | 1.00 | 45.67 | C |
| ATOM | 1650 | N | MET | A | 279 | 22.217 | 12.576 | 34.925 | 1.00 | 40.29 | N |
| ATOM | 1651 | CA | MET | A | 279 | 21.408 | 11.852 | 33.952 | 1.00 | 34.57 | C |
| ATOM | 1652 | C | MET | A | 279 | 20.055 | 11.448 | 34.522 | 1.00 | 34.43 | C |
| ATOM | 1653 | O | MET | A | 279 | 19.051 | 11.448 | 33.813 | 1.00 | 32.79 | O |
| ATOM | 1654 | CB | MET | A | 279 | 22.148 | 10.609 | 33.453 | 1.00 | 28.74 | C |
| ATOM | 1655 | CG | MET | A | 279 | 23.459 | 10.910 | 32.746 | 1.00 | 32.48 | C |
| ATOM | 1656 | SD | MET | A | 279 | 24.341 | 9.409 | 32.243 | 1.00 | 41.72 | S |
| ATOM | 1657 | CE | MET | A | 279 | 25.815 | 10.146 | 31.549 | 1.00 | 34.30 | C |
| ATOM | 1658 | N | GLY | A | 280 | 20.036 | 11.097 | 35.800 | 1.00 | 30.04 | N |
| ATOM | 1659 | CA | GLY | A | 280 | 18.799 | 10.731 | 36.465 | 1.00 | 29.03 | C |
| ATOM | 1660 | C | GLY | A | 280 | 17.871 | 11.915 | 36.655 | 1.00 | 29.03 | C |
| ATOM | 1661 | O | GLY | A | 280 | 16.664 | 11.819 | 36.433 | 1.00 | 28.08 | O |
| ATOM | 1662 | N | VAL | A | 281 | 18.439 | 13.043 | 37.061 | 1.00 | 26.88 | N |
| ATOM | 1663 | CA | VAL | A | 281 | 17.648 | 14.243 | 37.289 | 1.00 | 32.05 | C |
| ATOM | 1664 | C | VAL | A | 281 | 17.091 | 14.778 | 35.970 | 1.00 | 36.24 | C |
| ATOM | 1665 | O | VAL | A | 281 | 15.971 | 15.288 | 35.923 | 1.00 | 33.22 | O |
| ATOM | 1666 | CB | VAL | A | 281 | 18.460 | 15.346 | 38.030 | 1.00 | 31.84 | C |
| ATOM | 1667 | CG1 | VAL | A | 281 | 17.626 | 16.596 | 38.206 | 1.00 | 25.41 | C |
| ATOM | 1668 | CG2 | VAL | A | 281 | 18.922 | 14.852 | 39.386 | 1.00 | 23.30 | C |
| ATOM | 1669 | N | PHE | A | 282 | 17.870 | 14.660 | 34.895 | 1.00 | 34.19 | N |
| ATOM | 1670 | CA | PHE | A | 282 | 17.397 | 15.077 | 33.577 | 1.00 | 23.76 | C |
| ATOM | 1671 | C | PHE | A | 282 | 16.176 | 14.260 | 33.171 | 1.00 | 23.79 | C |
| ATOM | 1672 | O | PHE | A | 282 | 15.193 | 14.808 | 32.715 | 1.00 | 24.64 | O |
| ATOM | 1673 | CB | PHE | A | 282 | 18.504 | 14.942 | 32.536 | 1.00 | 23.90 | C |
| ATOM | 1674 | CG | PHE | A | 282 | 18.055 | 15.227 | 31.130 | 1.00 | 28.01 | C |
| ATOM | 1675 | CD1 | PHE | A | 282 | 18.209 | 16.505 | 30.581 | 1.00 | 22.81 | C |
| ATOM | 1676 | CD2 | PHE | A | 282 | 17.478 | 14.223 | 30.350 | 1.00 | 15.85 | C |
| ATOM | 1677 | CE1 | PHE | A | 282 | 17.801 | 16.781 | 29.277 | 1.00 | 17.51 | C |
| ATOM | 1678 | CE2 | PHE | A | 282 | 17.059 | 14.491 | 29.056 | 1.00 | 19.68 | C |
| ATOM | 1679 | CZ | PHE | A | 282 | 17.224 | 15.775 | 28.514 | 1.00 | 18.96 | C |
| ATOM | 1680 | N | THR | A | 283 | 16.254 | 12.947 | 33.348 | 1.00 | 24.28 | N |
| ATOM | 1681 | CA | THR | A | 283 | 15.135 | 12.058 | 33.086 | 1.00 | 24.17 | C |
| ATOM | 1682 | C | THR | A | 283 | 13.919 | 12.426 | 33.934 | 1.00 | 28.85 | C |
| ATOM | 1683 | O | THR | A | 283 | 12.833 | 12.634 | 33.412 | 1.00 | 36.45 | O |
| ATOM | 1684 | CB | THR | A | 283 | 15.518 | 10.581 | 33.338 | 1.00 | 28.30 | C |
| ATOM | 1685 | OG1 | THR | A | 283 | 16.713 | 10.265 | 32.608 | 1.00 | 27.27 | O |
| ATOM | 1686 | CG2 | THR | A | 283 | 14.391 | 9.628 | 32.907 | 1.00 | 29.15 | C |
| ATOM | 1687 | N | LEU | A | 284 | 14.094 | 12.506 | 35.243 | 1.00 | 30.79 | N |
| ATOM | 1688 | CA | LEU | A | 284 | 12.993 | 12.908 | 36.110 | 1.00 | 38.11 | C |
| ATOM | 1689 | C | LEU | A | 284 | 12.342 | 14.240 | 35.698 | 1.00 | 36.34 | C |
| ATOM | 1690 | O | LEU | A | 284 | 11.169 | 14.462 | 35.972 | 1.00 | 38.76 | O |
| ATOM | 1691 | CB | LEU | A | 284 | 13.450 | 12.980 | 37.570 | 1.00 | 39.24 | C |
| ATOM | 1692 | CG | LEU | A | 284 | 13.911 | 11.659 | 38.174 | 1.00 | 49.58 | C |
| ATOM | 1693 | CD1 | LEU | A | 284 | 14.251 | 11.856 | 39.636 | 1.00 | 46.06 | C |
| ATOM | 1694 | CD2 | LEU | A | 284 | 12.854 | 10.569 | 37.998 | 1.00 | 40.05 | C |
| ATOM | 1695 | N | CYS | A | 285 | 13.089 | 15.122 | 35.042 | 1.00 | 22.72 | N |
| ATOM | 1696 | CA | CYS | A | 285 | 12.576 | 16.460 | 34.774 | 1.00 | 23.63 | C |
| ATOM | 1697 | C | CYS | A | 285 | 11.844 | 16.601 | 33.450 | 1.00 | 31.72 | C |
| ATOM | 1698 | O | CYS | A | 285 | 11.064 | 17.538 | 33.283 | 1.00 | 33.54 | O |
| ATOM | 1699 | CB | CYS | A | 285 | 13.705 | 17.484 | 34.800 | 1.00 | 33.20 | C |
| ATOM | 1700 | SG | CYS | A | 285 | 14.119 | 18.141 | 36.417 | 1.00 | 38.70 | S |
| ATOM | 1701 | N | TRP | A | 286 | 12.108 | 15.689 | 32.511 | 1.00 | 30.43 | N |
| ATOM | 1702 | CA | TRP | A | 286 | 11.595 | 15.805 | 31.146 | 1.00 | 20.54 | C |
| ATOM | 1703 | C | TRP | A | 286 | 10.657 | 14.676 | 30.769 | 1.00 | 25.41 | C |
| ATOM | 1704 | O | TRP | A | 286 | 9.769 | 14.851 | 29.946 | 1.00 | 28.19 | O |
| ATOM | 1705 | CB | TRP | A | 286 | 12.744 | 15.867 | 30.145 | 1.00 | 19.64 | C |
| ATOM | 1706 | CG | TRP | A | 286 | 13.288 | 17.235 | 29.972 | 1.00 | 19.89 | C |
| ATOM | 1707 | CD1 | TRP | A | 286 | 14.525 | 17.667 | 30.321 | 1.00 | 17.16 | C |
| ATOM | 1708 | CD2 | TRP | A | 286 | 12.604 | 18.371 | 29.428 | 1.00 | 19.43 | C |
| ATOM | 1709 | NE1 | TRP | A | 286 | 14.662 | 19.000 | 30.017 | 1.00 | 17.58 | N |
| ATOM | 1710 | CE2 | TRP | A | 286 | 13.499 | 19.456 | 29.464 | 1.00 | 15.44 | C |
| ATOM | 1711 | CE3 | TRP | A | 286 | 11.325 | 18.574 | 28.909 | 1.00 | 19.34 | C |
| ATOM | 1712 | CZ2 | TRP | A | 286 | 13.159 | 20.727 | 28.999 | 1.00 | 13.71 | C |
| ATOM | 1713 | CZ3 | TRP | A | 286 | 10.989 | 19.835 | 28.443 | 1.00 | 18.63 | C |
| ATOM | 1714 | CH2 | TRP | A | 286 | 11.903 | 20.894 | 28.494 | 1.00 | 17.00 | C |
| ATOM | 1715 | N | LEU | A | 287 | 10.852 | 13.515 | 31.369 | 1.00 | 22.87 | N |
| ATOM | 1716 | CA | LEU | A | 287 | 9.992 | 12.382 | 31.074 | 1.00 | 30.41 | C |
| ATOM | 1717 | C | LEU | A | 287 | 8.499 | 12.708 | 31.280 | 1.00 | 29.75 | C |
| ATOM | 1718 | O | LEU | A | 287 | 7.657 | 12.271 | 30.495 | 1.00 | 35.86 | O |
| ATOM | 1719 | CB | LEU | A | 287 | 10.424 | 11.147 | 31.878 | 1.00 | 33.91 | C |
| ATOM | 1720 | CG | LEU | A | 287 | 9.725 | 9.820 | 31.563 | 1.00 | 36.53 | C |
| ATOM | 1721 | CD1 | LEU | A | 287 | 9.610 | 9.607 | 30.071 | 1.00 | 23.45 | C |
| ATOM | 1722 | CD2 | LEU | A | 287 | 10.470 | 8.666 | 32.216 | 1.00 | 34.38 | C |
| ATOM | 1723 | N | PRO | A | 288 | 8.162 | 13.482 | 32.321 | 1.00 | 29.36 | N |
| ATOM | 1724 | CA | PRO | A | 288 | 6.731 | 13.798 | 32.460 | 1.00 | 28.86 | C |
| ATOM | 1725 | C | PRO | A | 288 | 6.199 | 14.544 | 31.246 | 1.00 | 32.00 | C |
| ATOM | 1726 | O | PRO | A | 288 | 5.182 | 14.143 | 30.673 | 1.00 | 36.36 | O |
| ATOM | 1727 | CB | PRO | A | 288 | 6.676 | 14.703 | 33.700 | 1.00 | 29.48 | C |
| ATOM | 1728 | CG | PRO | A | 288 | 7.923 | 14.353 | 34.489 | 1.00 | 33.48 | C |
| ATOM | 1729 | CD | PRO | A | 288 | 8.966 | 13.967 | 33.461 | 1.00 | 33.01 | C |
| ATOM | 1730 | N | PHE | A | 289 | 6.882 | 15.610 | 30.844 | 1.00 | 23.17 | N |
| ATOM | 1731 | CA | PHE | A | 289 | 6.429 | 16.395 | 29.702 | 1.00 | 26.42 | C |
| ATOM | 1732 | C | PHE | A | 289 | 6.283 | 15.598 | 28.384 | 1.00 | 34.74 | C |
| ATOM | 1733 | O | PHE | A | 289 | 5.391 | 15.893 | 27.584 | 1.00 | 33.37 | O |
| ATOM | 1734 | CB | PHE | A | 289 | 7.330 | 17.613 | 29.509 | 1.00 | 24.07 | C |
| ATOM | 1735 | CG | PHE | A | 289 | 7.167 | 18.288 | 28.178 | 1.00 | 30.41 | C |
| ATOM | 1736 | CD1 | PHE | A | 289 | 6.371 | 19.420 | 28.045 | 1.00 | 32.60 | C |
| ATOM | 1737 | CD2 | PHE | A | 289 | 7.823 | 17.799 | 27.052 | 1.00 | 28.30 | C |
| ATOM | 1738 | CE1 | PHE | A | 289 | 6.228 | 20.056 | 26.805 | 1.00 | 32.75 | C |
| ATOM | 1739 | CE2 | PHE | A | 289 | 7.687 | 18.430 | 25.807 | 1.00 | 30.75 | C |
| ATOM | 1740 | CZ | PHE | A | 289 | 6.893 | 19.561 | 25.685 | 1.00 | 28.80 | C |
| ATOM | 1741 | N | PHE | A | 290 | 7.149 | 14.606 | 28.152 | 1.00 | 31.78 | N |
| ATOM | 1742 | CA | PHE | A | 290 | 7.078 | 13.796 | 26.930 | 1.00 | 29.16 | C |
| ATOM | 1743 | C | PHE | A | 290 | 6.060 | 12.684 | 27.063 | 1.00 | 34.27 | C |
| ATOM | 1744 | O | PHE | A | 290 | 5.477 | 12.250 | 26.069 | 1.00 | 30.84 | O |
| ATOM | 1745 | CB | PHE | A | 290 | 8.446 | 13.213 | 26.516 | 1.00 | 29.99 | C |
| ATOM | 1746 | CG | PHE | A | 290 | 9.354 | 14.217 | 25.877 | 1.00 | 25.86 | C |
| ATOM | 1747 | CD1 | PHE | A | 290 | 10.367 | 14.821 | 26.610 | 1.00 | 25.42 | C |
| ATOM | 1748 | CD2 | PHE | A | 290 | 9.177 | 14.586 | 24.558 | 1.00 | 26.14 | C |
| ATOM | 1749 | CE1 | PHE | A | 290 | 11.192 | 15.769 | 26.040 | 1.00 | 20.05 | C |
| ATOM | 1750 | CE2 | PHE | A | 290 | 10.000 | 15.543 | 23.978 | 1.00 | 33.58 | C |
| ATOM | 1751 | CZ | PHE | A | 290 | 11.005 | 16.138 | 24.726 | 1.00 | 26.98 | C |
| ATOM | 1752 | N | LEU | A | 291 | 5.844 | 12.210 | 28.285 | 1.00 | 30.18 | N |
| ATOM | 1753 | CA | LEU | A | 291 | 4.770 | 11.249 | 28.493 | 1.00 | 38.55 | C |
| ATOM | 1754 | C | LEU | A | 291 | 3.410 | 11.910 | 28.260 | 1.00 | 37.01 | C |
| ATOM | 1755 | O | LEU | A | 291 | 2.524 | 11.326 | 27.642 | 1.00 | 46.76 | O |
| ATOM | 1756 | CB | LEU | A | 291 | 4.853 | 10.599 | 29.871 | 1.00 | 36.31 | C |
| ATOM | 1757 | CG | LEU | A | 291 | 5.868 | 9.466 | 29.883 | 1.00 | 35.53 | C |
| ATOM | 1758 | CD1 | LEU | A | 291 | 5.740 | 8.623 | 31.134 | 1.00 | 21.38 | C |
| ATOM | 1759 | CD2 | LEU | A | 291 | 5.655 | 8.616 | 28.648 | 1.00 | 38.33 | C |
| ATOM | 1760 | N | VAL | A | 292 | 3.258 | 13.141 | 28.728 | 1.00 | 30.20 | N |
| ATOM | 1761 | CA | VAL | A | 292 | 2.011 | 13.868 | 28.532 | 1.00 | 34.07 | C |
| ATOM | 1762 | C | VAL | A | 292 | 1.838 | 14.258 | 27.065 | 1.00 | 32.13 | C |
| ATOM | 1763 | O | VAL | A | 292 | 0.735 | 14.554 | 26.604 | 1.00 | 34.65 | O |
| ATOM | 1764 | CB | VAL | A | 292 | 1.931 | 15.087 | 29.492 | 1.00 | 30.82 | C |
| ATOM | 1765 | CG1 | VAL | A | 292 | 1.535 | 16.353 | 28.771 | 1.00 | 29.45 | C |
| ATOM | 1766 | CG2 | VAL | A | 292 | 0.987 | 14.776 | 30.627 | 1.00 | 29.79 | C |
| ATOM | 1767 | N | ASN | A | 293 | 2.943 | 14.223 | 26.330 | 1.00 | 32.74 | N |
| ATOM | 1768 | CA | ASN | A | 293 | 2.940 | 14.592 | 24.931 | 1.00 | 33.24 | C |
| ATOM | 1769 | C | ASN | A | 293 | 2.428 | 13.456 | 24.060 | 1.00 | 34.83 | C |
| ATOM | 1770 | O | ASN | A | 293 | 1.787 | 13.699 | 23.052 | 1.00 | 35.17 | O |
| ATOM | 1771 | CB | ASN | A | 293 | 4.335 | 15.008 | 24.487 | 1.00 | 38.82 | C |
| ATOM | 1772 | CG | ASN | A | 293 | 4.314 | 16.205 | 23.558 | 1.00 | 49.20 | C |
| ATOM | 1773 | OD1 | ASN | A | 293 | 3.829 | 17.281 | 23.921 | 1.00 | 40.28 | O |
| ATOM | 1774 | ND2 | ASN | A | 293 | 4.853 | 16.032 | 22.356 | 1.00 | 53.57 | N |
| ATOM | 1775 | N | ILE | A | 294 | 2.711 | 12.216 | 24.452 | 1.00 | 41.48 | N |
| ATOM | 1776 | CA | ILE | A | 294 | 2.213 | 11.051 | 23.727 | 1.00 | 36.70 | C |
| ATOM | 1777 | C | ILE | A | 294 | 0.738 | 10.867 | 24.037 | 1.00 | 39.42 | C |
| ATOM | 1778 | O | ILE | A | 294 | −0.020 | 10.345 | 23.219 | 1.00 | 37.96 | O |
| ATOM | 1779 | CB | ILE | A | 294 | 2.982 | 9.777 | 24.111 | 1.00 | 36.72 | C |
| ATOM | 1780 | CG1 | ILE | A | 294 | 4.425 | 9.889 | 23.646 | 1.00 | 43.21 | C |
| ATOM | 1781 | CG2 | ILE | A | 294 | 2.337 | 8.536 | 23.506 | 1.00 | 26.12 | C |
| ATOM | 1782 | CD1 | ILE | A | 294 | 5.273 | 8.709 | 24.050 | 1.00 | 55.44 | C |
| ATOM | 1783 | N | VAL | A | 295 | 0.331 | 11.316 | 25.219 | 1.00 | 29.39 | N |
| ATOM | 1784 | CA | VAL | A | 295 | −1.060 | 11.195 | 25.615 | 1.00 | 32.53 | C |
| ATOM | 1785 | C | VAL | A | 295 | −1.962 | 12.164 | 24.837 | 1.00 | 39.57 | C |
| ATOM | 1786 | O | VAL | A | 295 | −3.084 | 11.798 | 24.474 | 1.00 | 35.13 | O |
| ATOM | 1787 | CB | VAL | A | 295 | −1.238 | 11.336 | 27.145 | 1.00 | 38.23 | C |
| ATOM | 1788 | CG1 | VAL | A | 295 | −2.718 | 11.418 | 27.532 | 1.00 | 38.04 | C |
| ATOM | 1789 | CG2 | VAL | A | 295 | −0.570 | 10.172 | 27.846 | 1.00 | 30.65 | C |
| ATOM | 1790 | N | ASN | A | 296 | −1.491 | 13.383 | 24.562 | 1.00 | 32.31 | N |
| ATOM | 1791 | CA | ASN | A | 296 | −2.304 | 14.290 | 23.739 | 1.00 | 37.25 | C |
| ATOM | 1792 | C | ASN | A | 296 | −2.444 | 13.803 | 22.322 | 1.00 | 28.75 | C |
| ATOM | 1793 | O | ASN | A | 296 | −3.304 | 14.262 | 21.590 | 1.00 | 28.05 | O |
| ATOM | 1794 | CB | ASN | A | 296 | −1.795 | 15.732 | 23.745 | 1.00 | 34.25 | C |
| ATOM | 1795 | CG | ASN | A | 296 | −2.418 | 16.548 | 24.854 | 1.00 | 68.72 | C |
| ATOM | 1796 | OD1 | ASN | A | 296 | −3.610 | 16.867 | 24.823 | 1.00 | 68.31 | O |
| ATOM | 1797 | ND2 | ASN | A | 296 | −1.623 | 16.864 | 25.863 | 1.00 | 78.60 | N |
| ATOM | 1798 | N | VAL | A | 297 | −1.583 | 12.871 | 21.941 | 1.00 | 31.44 | N |
| ATOM | 1799 | CA | VAL | A | 297 | −1.654 | 12.283 | 20.623 | 1.00 | 40.37 | C |
| ATOM | 1800 | C | VAL | A | 297 | −2.849 | 11.335 | 20.552 | 1.00 | 38.56 | C |
| ATOM | 1801 | O | VAL | A | 297 | −3.630 | 11.386 | 19.602 | 1.00 | 43.26 | O |
| ATOM | 1802 | CB | VAL | A | 297 | −0.333 | 11.575 | 20.249 | 1.00 | 41.77 | C |
| ATOM | 1803 | CG1 | VAL | A | 297 | −0.565 | 10.541 | 19.145 | 1.00 | 35.23 | C |
| ATOM | 1804 | CG2 | VAL | A | 297 | 0.716 | 12.607 | 19.835 | 1.00 | 27.83 | C |
| ATOM | 1805 | N | PHE | A | 298 | −3.002 | 10.510 | 21.583 | 1.00 | 30.68 | N |
| ATOM | 1806 | CA | PHE | A | 298 | −4.074 | 9.515 | 21.643 | 1.00 | 40.10 | C |
| ATOM | 1807 | C | PHE | A | 298 | −5.433 | 10.186 | 21.752 | 1.00 | 42.88 | C |
| ATOM | 1808 | O | PHE | A | 298 | −6.401 | 9.793 | 21.098 | 1.00 | 50.76 | O |
| ATOM | 1809 | CB | PHE | A | 29B | −3.842 | 8.563 | 22.820 | 1.00 | 40.28 | C |
| ATOM | 1810 | CG | PHE | A | 298 | −5.042 | 7.732 | 23.196 | 1.00 | 51.29 | C |
| ATOM | 1811 | CD1 | PHE | A | 298 | −5.526 | 6.750 | 22.347 | 1.00 | 52.77 | C |
| ATOM | 1812 | CD2 | PHE | A | 298 | −5.667 | 7.914 | 24.427 | 1.00 | 59.63 | C |
| ATOM | 1813 | CE1 | PHE | A | 298 | −6.629 | 5.983 | 22.709 | 1.00 | 59.59 | C |
| ATOM | 1814 | CE2 | PHE | A | 298 | −6.764 | 7.145 | 24.796 | 1.00 | 52.99 | C |
| ATOM | 1815 | CZ | PHE | A | 298 | −7.245 | 6.178 | 23.935 | 1.00 | 52.85 | C |
| ATOM | 1816 | N | ASN | A | 299 | −5.485 | 11.211 | 22.584 | 1.00 | 38.17 | N |
| ATOM | 1817 | CA | ASN | A | 299 | −6.692 | 11.963 | 22.821 | 1.00 | 33.80 | C |
| ATOM | 1818 | C | ASN | A | 299 | −6.252 | 13.335 | 23.290 | 1.00 | 33.16 | C |
| ATOM | 1819 | O | ASN | A | 299 | −5.761 | 13.488 | 24.397 | 1.00 | 36.04 | O |
| ATOM | 1820 | CB | ASN | A | 299 | −7.538 | 11.264 | 23.902 | 1.00 | 55.31 | C |
| ATOM | 1821 | CG | ASN | A | 299 | −8.950 | 11.869 | 24.074 | 1.00 | 56.13 | C |
| ATOM | 1822 | OD1 | ASN | A | 299 | −9.425 | 12.659 | 23.263 | 1.00 | 39.23 | O |
| ATOM | 1823 | ND2 | ASN | A | 299 | −9.620 | 11.472 | 25.146 | 1.00 | 64.78 | N |
| ATOM | 1824 | N | ARG | A | 300 | −6.391 | 14.335 | 22.433 | 1.00 | 41.37 | N |
| ATOM | 1825 | CA | ARG | A | 300 | −6.342 | 15.705 | 22.908 | 1.00 | 39.87 | C |
| ATOM | 1826 | C | ARG | A | 300 | −7.506 | 15.755 | 23.886 | 1.00 | 43.96 | C |
| ATOM | 1827 | O | ARG | A | 300 | −8.306 | 14.820 | 23.929 | 1.00 | 60.58 | O |
| ATOM | 1828 | CB | ARG | A | 300 | −6.509 | 16.675 | 21.734 | 1.00 | 46.81 | C |
| ATOM | 1829 | CG | ARG | A | 300 | −5.748 | 16.222 | 20.475 | 1.00 | 57.36 | C |
| ATOM | 1830 | CD | ARG | A | 300 | −5.202 | 17.383 | 19.637 | 1.00 | 78.04 | C |
| ATOM | 1831 | NE | ARG | A | 300 | −4.256 | 16.936 | 18.610 | 1.00 | 67.84 | N |
| ATOM | 1832 | CZ | ARG | A | 300 | −3.939 | 17.639 | 17.522 | 1.00 | 84.20 | C |
| ATOM | 1833 | NH1 | ARG | A | 300 | −4.495 | 18.827 | 17.305 | 1.00 | 78.04 | N |
| ATOM | 1834 | NH2 | ARG | A | 300 | −3.071 | 17.151 | 16.641 | 1.00 | 63.79 | N |
| ATOM | 1835 | N | ASP | A | 301 | −7.608 | 16.799 | 24.693 | 1.00 | 38.01 | N |
| ATOM | 1836 | CA | ASP | A | 301 | −8.663 | 16.860 | 25.728 | 1.00 | 48.40 | C |
| ATOM | 1837 | C | ASP | A | 301 | −8.390 | 15.974 | 26.963 | 1.00 | 42.36 | C |
| ATOM | 1838 | O | ASP | A | 301 | −8.778 | 16.323 | 28.073 | 1.00 | 55.68 | O |
| ATOM | 1839 | CB | ASP | A | 301 | −10.068 | 16.554 | 25.153 | 1.00 | 45.05 | C |
| ATOM | 1840 | CG | ASP | A | 301 | −10.513 | 17.563 | 24.077 | 1.00 | 58.21 | C |
| ATOM | 1841 | OD1 | ASP | A | 301 | −10.221 | 18.770 | 24.223 | 1.00 | 51.40 | O |
| ATOM | 1842 | OD2 | ASP | A | 301 | −11.165 | 17.151 | 23.083 | 1.00 | 51.99 | O |
| ATOM | 1843 | N | LEU | A | 302 | −7.708 | 14.847 | 26.778 | 1.00 | 48.76 | N |
| ATOM | 1844 | CA | LEU | A | 302 | −7.499 | 13.887 | 27.866 | 1.00 | 46.24 | C |
| ATOM | 1845 | C | LEU | A | 302 | −6.518 | 14.365 | 28.951 | 1.00 | 56.81 | C |
| ATOM | 1846 | O | LEU | A | 302 | −6.183 | 13.615 | 29.870 | 1.00 | 57.23 | O |
| ATOM | 1847 | CB | LEU | A | 302 | −7.038 | 12.539 | 27.297 | 1.00 | 47.16 | C |
| ATOM | 1848 | CG | LEU | A | 302 | −7.262 | 11.307 | 28.176 | 1.00 | 64.24 | C |
| ATOM | 1849 | CD1 | LEU | A | 302 | −8.674 | 10.780 | 28.013 | 1.00 | 73.09 | C |
| ATOM | 1850 | CD2 | LEU | A | 302 | −6.256 | 10.218 | 27.849 | 1.00 | 67.65 | C |
| ATOM | 1851 | N | VAL | A | 303 | −6.060 | 15.607 | 28.848 | 1.00 | 53.21 | N |
| ATOM | 1852 | CA | VAL | A | 303 | −5.088 | 16.146 | 29.797 | 1.00 | 47.03 | C |
| ATOM | 1853 | C | VAL | A | 303 | −4.986 | 17.662 | 29.673 | 1.00 | 48.63 | C |
| ATOM | 1854 | O | VAL | A | 303 | −4.795 | 18.194 | 28.578 | 1.00 | 52.65 | O |
| ATOM | 1855 | CB | VAL | A | 303 | −3.696 | 15.467 | 29.651 | 1.00 | 76.72 | C |
| ATOM | 1856 | CG1 | VAL | A | 303 | −2.597 | 16.489 | 29.339 | 1.00 | 52.44 | C |
| ATOM | 1857 | CG2 | VAL | A | 303 | −3.369 | 14.647 | 30.902 | 1.00 | 79.38 | C |
| ATOM | 1858 | N | PRO | A | 304 | −5.120 | 18.353 | 30.813 | 1.00 | 45.51 | N |
| ATOM | 1859 | CA | PRO | A | 304 | −5.423 | 19.784 | 30.968 | 1.00 | 42.15 | C |
| ATOM | 1860 | C | PRO | A | 304 | −4.234 | 20.698 | 30.753 | 1.00 | 50.04 | C |
| ATOM | 1861 | O | PRO | A | 304 | −3.168 | 20.480 | 31.337 | 1.00 | 52.26 | O |
| ATOM | 1862 | CB | PRO | A | 304 | −5.858 | 19.874 | 32.431 | 1.00 | 45.70 | C |
| ATOM | 1863 | CG | PRO | A | 304 | −5.055 | 18.788 | 33.089 | 1.00 | 45.19 | C |
| ATOM | 1864 | CD | PRO | A | 304 | −5.106 | 17.656 | 32.111 | 1.00 | 44.37 | C |
| ATOM | 1865 | N | ASP | A | 305 | −4.438 | 21.742 | 29.959 | 1.00 | 48.48 | N |
| ATOM | 1866 | CA | ASP | A | 305 | −3.359 | 22.652 | 29.578 | 1.00 | 50.77 | C |
| ATOM | 1867 | C | ASP | A | 305 | −2.421 | 23.018 | 30.738 | 1.00 | 53.64 | C |
| ATOM | 1868 | O | ASP | A | 305 | −1.201 | 23.065 | 30.566 | 1.00 | 53.35 | O |
| ATOM | 1869 | CB | ASP | A | 305 | −3.936 | 23.903 | 28.903 | 1.00 | 46.66 | C |
| ATOM | 1870 | CG | ASP | A | 305 | −4.797 | 23.565 | 27.676 | 1.00 | 80.33 | C |
| ATOM | 1871 | OD1 | ASP | A | 305 | −4.293 | 22.895 | 26.747 | 1.00 | 82.34 | O |
| ATOM | 1872 | OD2 | ASP | A | 305 | −5.981 | 23.966 | 27.638 | 1.00 | 76.24 | O |
| ATOM | 1873 | N | TRP | A | 306 | −2.982 | 23.258 | 31.919 | 1.00 | 55.32 | N |
| ATOM | 1874 | CA | TRP | A | 306 | −2.169 | 23.657 | 33.070 | 1.00 | 48.34 | C |
| ATOM | 1875 | C | TRP | A | 306 | −1.138 | 22.583 | 33.438 | 1.00 | 44.19 | C |
| ATOM | 1876 | O | TRP | A | 306 | −0.040 | 22.902 | 33.897 | 1.00 | 37.44 | O |
| ATOM | 1877 | CB | TRP | A | 306 | −3.049 | 24.007 | 34.291 | 1.00 | 38.86 | C |
| ATOM | 1878 | CG | TRP | A | 306 | −3.675 | 22.809 | 34.925 | 1.00 | 41.79 | C |
| ATOM | 1879 | CD1 | TRP | A | 306 | −4.932 | 22.330 | 34.700 | 1.00 | 42.75 | C |
| ATOM | 1880 | CD2 | TRP | A | 306 | −3.062 | 21.909 | 35.862 | 1.00 | 39.45 | C |
| ATOM | 1881 | NE1 | TRP | A | 306 | −5.142 | 21.192 | 35.446 | 1.00 | 44.90 | N |
| ATOM | 1882 | CE2 | TRP | A | 306 | −4.009 | 20.912 | 36.164 | 1.00 | 36.69 | C |
| ATOM | 1883 | CE3 | TRP | A | 306 | −1.799 | 21.843 | 36.464 | 1.00 | 50.61 | C |
| ATOM | 1884 | CZ2 | TRP | A | 306 | −3.741 | 19.867 | 37.050 | 1.00 | 39.13 | C |
| ATOM | 1885 | CZ3 | TRP | A | 306 | −1.533 | 20.803 | 37.349 | 1.00 | 47.84 | C |
| ATOM | 1886 | CH2 | TRP | A | 306 | −2.502 | 19.832 | 37.633 | 1.00 | 35.00 | C |
| ATOM | 1887 | N | LEU | A | 307 | −1.495 | 21.314 | 33.253 | 1.00 | 40.36 | N |
| ATOM | 1888 | CA | LEU | A | 307 | −0.572 | 20.234 | 33.565 | 1.00 | 43.17 | C |
| ATOM | 1889 | C | LEU | A | 307 | 0.559 | 20.220 | 32.532 | 1.00 | 42.75 | C |
| ATOM | 1890 | O | LEU | A | 307 | 1.667 | 19.736 | 32.780 | 1.00 | 34.32 | O |
| ATOM | 1891 | CB | LEU | A | 307 | −1.288 | 18.884 | 33.605 | 1.00 | 34.20 | C |
| ATOM | 1892 | CG | LEU | A | 307 | −0.356 | 17.700 | 33.904 | 1.00 | 38.15 | C |
| ATOM | 1893 | CD1 | LEU | A | 307 | 0.496 | 17.953 | 35.149 | 1.00 | 37.24 | C |
| ATOM | 1894 | CD2 | LEU | A | 307 | −1.131 | 16.409 | 34.051 | 1.00 | 44.32 | C |
| ATOM | 1895 | N | PHE | A | 308 | 0.272 | 20.774 | 31.368 | 1.00 | 40.30 | N |
| ATOM | 1896 | CA | PHE | A | 308 | 1.271 | 20.847 | 30.330 | 1.00 | 39.85 | C |
| ATOM | 1897 | C | PHE | A | 308 | 2.261 | 21.924 | 30.695 | 1.00 | 39.32 | C |
| ATOM | 1898 | O | PHE | A | 308 | 3.469 | 21.742 | 30.573 | 1.00 | 40.33 | O |
| ATOM | 1899 | CB | PHE | A | 308 | 0.622 | 21.177 | 28.993 | 1.00 | 40.57 | C |
| ATOM | 1900 | CG | PHE | A | 308 | 0.907 | 20.170 | 27.941 | 1.00 | 45.51 | C |
| ATOM | 1901 | CD1 | PHE | A | 308 | −0.118 | 19.478 | 27.328 | 1.00 | 46.52 | C |
| ATOM | 1902 | CD2 | PHE | A | 308 | 2.213 | 19.882 | 27.588 | 1.00 | 56.88 | C |
| ATOM | 1903 | CE1 | PHE | A | 308 | 0.154 | 18.539 | 26.365 | 1.00 | 43.73 | C |
| ATOM | 1904 | CE2 | PHE | A | 308 | 2.491 | 18.935 | 26.623 | 1.00 | 53.41 | C |
| ATOM | 1905 | CZ | PHE | A | 308 | 1.458 | 18.261 | 26.014 | 1.00 | 47.50 | C |
| ATOM | 1906 | N | VAL | A | 309 | 1.743 | 23.056 | 31.149 | 1.00 | 34.05 | N |
| ATOM | 1907 | CA | VAL | A | 309 | 2.607 | 24.162 | 31.490 | 1.00 | 31.94 | C |
| ATOM | 1908 | C | VAL | A | 309 | 3.512 | 23.742 | 32.636 | 1.00 | 36.51 | C |
| ATOM | 1909 | O | VAL | A | 309 | 4.712 | 24.028 | 32.632 | 1.00 | 38.80 | O |
| ATOM | 1910 | CB | VAL | A | 309 | 1.811 | 25.429 | 31.865 | 1.00 | 36.58 | C |
| ATOM | 1911 | CG1 | VAL | A | 309 | 2.714 | 26.435 | 32.538 | 1.00 | 31.61 | C |
| ATOM | 1912 | CG2 | VAL | A | 309 | 1.188 | 26.050 | 30.627 | 1.00 | 31.16 | C |
| ATOM | 1913 | N | ALA | A | 310 | 2.944 | 23.044 | 33.609 | 1.00 | 27.43 | N |
| ATOM | 1914 | CA | ALA | A | 310 | 3.721 | 22.657 | 34.777 | 1.00 | 31.61 | C |
| ATOM | 1915 | C | ALA | A | 310 | 4.849 | 21.710 | 34.411 | 1.00 | 29.82 | C |
| ATOM | 1916 | O | ALA | A | 310 | 5.999 | 21.954 | 34.753 | 1.00 | 28.17 | O |
| ATOM | 1917 | CB | ALA | A | 310 | 2.833 | 22.035 | 35.837 | 1.00 | 33.03 | C |
| ATOM | 1918 | N | PHE | A | 311 | 4.514 | 20.619 | 33.731 | 1.00 | 30.28 | N |
| ATOM | 1919 | CA | PHE | A | 311 | 5.521 | 19.662 | 33.289 | 1.00 | 29.01 | C |
| ATOM | 1920 | C | PHE | A | 311 | 6.588 | 20.283 | 32.390 | 1.00 | 24.16 | C |
| ATOM | 1921 | O | PHE | A | 311 | 7.755 | 19.901 | 32.462 | 1.00 | 19.79 | O |
| ATOM | 1922 | CB | PHE | A | 311 | 4.872 | 18.462 | 32.602 | 1.00 | 28.06 | C |
| ATOM | 1923 | CG | PHE | A | 311 | 4.267 | 17.489 | 33.557 | 1.00 | 30.77 | C |
| ATOM | 1924 | CD1 | PHE | A | 311 | 3.303 | 16.591 | 33.143 | 1.00 | 37.91 | C |
| ATOM | 1925 | CD2 | PHE | A | 311 | 4.647 | 17.488 | 34.883 | 1.00 | 31.39 | C |
| ATOM | 1926 | CE1 | PHE | A | 311 | 2.745 | 15.691 | 34.030 | 1.00 | 38.99 | C |
| ATOM | 1927 | CE2 | PHE | A | 311 | 4.092 | 16.594 | 35.777 | 1.00 | 32.21 | C |
| ATOM | 1928 | CZ | PHE | A | 311 | 3.142 | 15.694 | 35.351 | 1.00 | 27.81 | C |
| ATOM | 1929 | N | ASN | A | 312 | 6.193 | 21.243 | 31.559 | 1.00 | 22.04 | N |
| ATOM | 1930 | CA | ASN | A | 312 | 7.153 | 21.945 | 30.719 | 1.00 | 30.68 | C |
| ATOM | 1931 | C | ASN | A | 312 | 8.137 | 22.802 | 31.520 | 1.00 | 27.18 | C |
| ATOM | 1932 | O | ASN | A | 312 | 9.302 | 22.942 | 31.142 | 1.00 | 27.28 | O |
| ATOM | 1933 | CB | ASN | A | 312 | 6.448 | 22.796 | 29.658 | 1.00 | 27.40 | C |
| ATOM | 1934 | CG | ASN | A | 312 | 7.297 | 22.990 | 28.399 | 1.00 | 24.90 | C |
| ATOM | 1935 | OD1 | ASN | A | 312 | 6.808 | 23.455 | 27.375 | 1.00 | 26.88 | O |
| ATOM | 1936 | ND2 | ASN | A | 312 | 8.570 | 22.630 | 28.478 | 1.00 | 30.58 | N |
| ATOM | 1937 | N | TRP | A | 313 | 7.671 | 23.373 | 32.621 | 1.00 | 26.32 | N |
| ATOM | 1938 | CA | TRP | A | 313 | 8.535 | 24.187 | 33.461 | 1.00 | 23.34 | C |
| ATOM | 1939 | C | TRP | A | 313 | 9.364 | 23.320 | 34.364 | 1.00 | 21.46 | C |
| ATOM | 1940 | O | TRP | A | 313 | 10.449 | 23.723 | 34.785 | 1.00 | 27.58 | O |
| ATOM | 1941 | CB | TRP | A | 313 | 7.736 | 25.225 | 34.256 | 1.00 | 29.94 | C |
| ATOM | 1942 | CG | TRP | A | 313 | 7.329 | 26.352 | 33.370 | 1.00 | 37.79 | C |
| ATOM | 1943 | CD1 | TRP | A | 313 | 6.225 | 26.401 | 32.559 | 1.00 | 34.07 | C |
| ATOM | 1944 | CD2 | TRP | A | 313 | 8.041 | 27.576 | 33.154 | 1.00 | 37.87 | C |
| ATOM | 1945 | NE1 | TRP | A | 313 | 6.200 | 27.588 | 31.866 | 1.00 | 38.51 | N |
| ATOM | 1946 | CE2 | TRP | A | 313 | 7.303 | 28.329 | 32.208 | 1.00 | 40.02 | C |
| ATOM | 1947 | CE3 | TRP | A | 313 | 9.224 | 28.116 | 33.673 | 1.00 | 36.00 | C |
| ATOM | 1948 | CZ2 | TRP | A | 313 | 7.710 | 29.594 | 31.769 | 1.00 | 32.64 | C |
| ATOM | 1949 | CZ3 | TRP | A | 313 | 9.626 | 29.370 | 33.237 | 1.00 | 45.28 | C |
| ATOM | 1950 | CH2 | TRP | A | 313 | 8.867 | 30.096 | 32.290 | 1.00 | 35.80 | C |
| ATOM | 1951 | N | LEU | A | 314 | 8.870 | 22.123 | 34.655 | 1.00 | 14.43 | N |
| ATOM | 1952 | CA | LEU | A | 314 | 9.663 | 21.177 | 35.419 | 1.00 | 18.61 | C |
| ATOM | 1953 | C | LEU | A | 314 | 10.956 | 20.893 | 34.648 | 1.00 | 30.32 | C |
| ATOM | 1954 | O | LEU | A | 314 | 12.055 | 20.939 | 35.208 | 1.00 | 30.52 | O |
| ATOM | 1955 | CB | LEU | A | 314 | 8.892 | 19.892 | 35.658 | 1.00 | 21.05 | C |
| ATOM | 1956 | CG | LEU | A | 314 | 9.790 | 18.803 | 36.234 | 1.00 | 28.28 | C |
| ATOM | 1957 | CD1 | LEU | A | 314 | 10.455 | 19.297 | 37.511 | 1.00 | 18.78 | C |
| ATOM | 1958 | CD2 | LEU | A | 314 | 9.007 | 17.520 | 36.466 | 1.00 | 26.58 | C |
| ATOM | 1959 | N | GLY | A | 315 | 10.812 | 20.616 | 33.357 | 1.00 | 22.34 | N |
| ATOM | 1960 | CA | GLY | A | 315 | 11.943 | 20.462 | 32.465 | 1.00 | 22.45 | C |
| ATOM | 1961 | C | GLY | A | 315 | 12.849 | 21.678 | 32.305 | 1.00 | 24.40 | C |
| ATOM | 1962 | O | GLY | A | 315 | 14.074 | 21.533 | 32.208 | 1.00 | 21.05 | O |
| ATOM | 1963 | N | TYR | A | 316 | 12.278 | 22.881 | 32.249 | 1.00 | 21.26 | N |
| ATOM | 1964 | CA | TYR | A | 316 | 13.127 | 24.055 | 32.185 | 1.00 | 20.00 | C |
| ATOM | 1965 | C | TYR | A | 316 | 13.940 | 24.146 | 33.464 | 1.00 | 25.77 | C |
| ATOM | 1966 | O | TYR | A | 316 | 15.116 | 24.483 | 33.432 | 1.00 | 31.20 | O |
| ATOM | 1967 | CB | TYR | A | 316 | 12.337 | 25.348 | 32.029 | 1.00 | 26.18 | C |
| ATOM | 1968 | CG | TYR | A | 316 | 11.676 | 25.589 | 30.690 | 1.00 | 30.32 | C |
| ATOM | 1969 | CD1 | TYR | A | 316 | 10.502 | 26.340 | 30.623 | 1.00 | 25.91 | C |
| ATOM | 1970 | CD2 | TYR | A | 316 | 12.214 | 25.085 | 29.495 | 1.00 | 24.72 | C |
| ATOM | 1971 | CE1 | TYR | A | 316 | 9.876 | 26.579 | 29.425 | 1.00 | 32.87 | C |
| ATOM | 1972 | CE2 | TYR | A | 316 | 11.583 | 25.322 | 28.268 | 1.00 | 25.69 | C |
| ATOM | 1973 | CZ | TYR | A | 316 | 10.405 | 26.069 | 28.251 | 1.00 | 35.54 | C |
| ATOM | 1974 | OH | TYR | A | 316 | 9.727 | 26.336 | 27.087 | 1.00 | 23.78 | O |
| ATOM | 1975 | N | ALA | A | 317 | 13.316 | 23.858 | 34.601 | 1.00 | 27.10 | N |
| ATOM | 1976 | CA | ALA | A | 317 | 14.009 | 24.011 | 35.880 | 1.00 | 31.56 | C |
| ATOM | 1977 | C | ALA | A | 317 | 15.267 | 23.141 | 35.925 | 1.00 | 30.12 | C |
| ATOM | 1978 | O | ALA | A | 317 | 16.224 | 23.449 | 36.633 | 1.00 | 36.10 | O |
| ATOM | 1979 | CB | ALA | A | 317 | 13.077 | 23.717 | 37.062 | 1.00 | 25.70 | C |
| ATOM | 1980 | N | ASN | A | 318 | 15.263 | 22.066 | 35.148 | 1.00 | 25.92 | N |
| ATOM | 1981 | CA | ASN | A | 318 | 16.436 | 21.222 | 34.997 | 1.00 | 28.36 | C |
| ATOM | 1982 | C | ASN | A | 318 | 17.718 | 21.986 | 34.639 | 1.00 | 31.01 | C |
| ATOM | 1983 | O | ASN | A | 318 | 18.802 | 21.587 | 35.051 | 1.00 | 38.96 | O |
| ATOM | 1984 | CB | ASN | A | 318 | 16.175 | 20.133 | 33.958 | 1.00 | 29.44 | C |
| ATOM | 1985 | CG | ASN | A | 318 | 17.347 | 19.183 | 33.804 | 1.00 | 29.62 | C |
| ATOM | 1986 | OD1 | ASN | A | 318 | 17.396 | 18.146 | 34.458 | 1.00 | 34.97 | O |
| ATOM | 1987 | ND2 | ASN | A | 318 | 18.297 | 19.532 | 32.933 | 1.00 | 29.17 | N |
| ATOM | 1988 | N | SER | A | 319 | 17.604 | 23.069 | 33.873 | 1.00 | 25.40 | N |
| ATOM | 1989 | CA | SER | A | 319 | 18.776 | 23.863 | 33.496 | 1.00 | 29.82 | C |
| ATOM | 1990 | C | SER | A | 319 | 19.453 | 24.537 | 34.696 | 1.00 | 34.93 | C |
| ATOM | 1991 | O | SER | A | 319 | 20.597 | 24.987 | 34.599 | 1.00 | 28.00 | O |
| ATOM | 1992 | CB | SER | A | 319 | 18.411 | 24.938 | 32.469 | 1.00 | 26.81 | C |
| ATOM | 1993 | OG | SER | A | 319 | 18.082 | 24.378 | 31.220 | 1.00 | 27.46 | O |
| ATOM | 1994 | N | ALA | A | 320 | 18.738 | 24.610 | 35.818 | 1.00 | 34.25 | N |
| ATOM | 1995 | CA | ALA | A | 320 | 19.261 | 25.240 | 37.027 | 1.00 | 33.94 | C |
| ATOM | 1996 | C | ALA | A | 320 | 19.871 | 24.235 | 38.017 | 1.00 | 32.35 | C |
| ATOM | 1997 | O | ALA | A | 320 | 20.658 | 24.609 | 38.876 | 1.00 | 33.27 | O |
| ATOM | 1998 | CB | ALA | A | 320 | 18.182 | 26.075 | 37.695 | 1.00 | 28.56 | C |
| ATOM | 1999 | N | MET | A | 321 | 19.527 | 22.961 | 37.869 | 1.00 | 29.51 | N |
| ATOM | 2000 | CA | MET | A | 321 | 20.030 | 21.913 | 38.756 | 1.00 | 35.23 | C |
| ATOM | 2001 | C | MET | A | 321 | 21.503 | 21.528 | 38.619 | 1.00 | 36.29 | C |
| ATOM | 2002 | O | MET | A | 321 | 22.172 | 21.264 | 39.614 | 1.00 | 39.19 | O |
| ATOM | 2003 | CB | MET | A | 321 | 19.184 | 20.657 | 38.603 | 1.00 | 36.04 | C |
| ATOM | 2004 | CG | MET | A | 321 | 17.824 | 20.789 | 39.226 | 1.00 | 43.97 | C |
| ATOM | 2005 | SD | MET | A | 321 | 16.684 | 19.595 | 38.548 | 1.00 | 52.50 | S |
| ATOM | 2006 | CE | MET | A | 321 | 15.171 | 20.088 | 39.381 | 1.00 | 40.72 | C |
| ATOM | 2007 | N | ASN | A | 322 | 22.009 | 21.458 | 37.397 | 1.00 | 38.22 | N |
| ATOM | 2008 | CA | ASN | A | 322 | 23.354 | 20.924 | 37.197 | 1.00 | 38.64 | C |
| ATOM | 2009 | C | ASN | A | 322 | 24.455 | 21.584 | 38.041 | 1.00 | 40.39 | C |
| ATOM | 2010 | O | ASN | A | 322 | 25.216 | 20.886 | 38.708 | 1.00 | 41.43 | O |
| ATOM | 2011 | CB | ASN | A | 322 | 23.731 | 20.893 | 35.710 | 1.00 | 49.34 | C |
| ATOM | 2012 | CG | ASN | A | 322 | 23.128 | 19.706 | 34.980 | 1.00 | 40.88 | C |
| ATOM | 2013 | OD1 | ASN | A | 322 | 21.940 | 19.427 | 35.102 | 1.00 | 46.73 | O |
| ATOM | 2014 | ND2 | ASN | A | 322 | 23.947 | 19.006 | 34.213 | 1.00 | 36.87 | N |
| ATOM | 2015 | N | PRO | A | 323 | 24.543 | 22.926 | 38.020 | 1.00 | 39.71 | N |
| ATOM | 2016 | CA | PRO | A | 323 | 25.602 | 23.590 | 38.791 | 1.00 | 42.67 | C |
| ATOM | 2017 | C | PRO | A | 323 | 25.528 | 23.231 | 40.280 | 1.00 | 41.51 | C |
| ATOM | 2018 | O | PRO | A | 323 | 26.538 | 22.909 | 40.903 | 1.00 | 42.81 | O |
| ATOM | 2019 | CB | PRO | A | 323 | 25.317 | 25.083 | 38.574 | 1.00 | 40.26 | C |
| ATOM | 2020 | CG | PRO | A | 323 | 24.495 | 25.148 | 37.332 | 1.00 | 43.40 | C |
| ATOM | 2021 | CD | PRO | A | 323 | 23.677 | 23.891 | 37.322 | 1.00 | 43.03 | C |
| ATOM | 2022 | N | ILE | A | 324 | 24.329 | 23.277 | 40.838 | 1.00 | 29.28 | N |
| ATOM | 2023 | CA | ILE | A | 324 | 24.123 | 22.792 | 42.187 | 1.00 | 37.13 | C |
| ATOM | 2024 | C | ILE | A | 324 | 24.746 | 21.402 | 42.366 | 1.00 | 35.21 | C |
| ATOM | 2025 | O | ILE | A | 324 | 25.602 | 21.209 | 43.216 | 1.00 | 44.57 | O |
| ATOM | 2026 | CB | ILE | A | 324 | 22.618 | 22.785 | 42.557 | 1.00 | 37.02 | C |
| ATOM | 2027 | CG1 | ILE | A | 324 | 22.136 | 24.215 | 42.828 | 1.00 | 27.30 | C |
| ATOM | 2028 | CG2 | ILE | A | 324 | 22.356 | 21.879 | 43.759 | 1.00 | 28.99 | C |
| ATOM | 2029 | CD1 | ILE | A | 324 | 20.626 | 24.373 | 42.836 | 1.00 | 38.99 | C |
| ATOM | 2030 | N | ILE | A | 325 | 24.327 | 20.437 | 41.560 | 1.00 | 36.21 | N |
| ATOM | 2031 | CA | ILE | A | 325 | 24.866 | 19.079 | 41.660 | 1.00 | 47.07 | C |
| ATOM | 2032 | C | ILE | A | 325 | 26.412 | 18.992 | 41.622 | 1.00 | 46.54 | C |
| ATOM | 2033 | O | ILE | A | 325 | 27.001 | 18.111 | 42.249 | 1.00 | 31.80 | O |
| ATOM | 2034 | CB | ILE | A | 325 | 24.250 | 18.172 | 40.567 | 1.00 | 39.87 | C |
| ATOM | 2035 | CG1 | ILE | A | 325 | 22.734 | 18.132 | 40.722 | 1.00 | 31.25 | C |
| ATOM | 2036 | CG2 | ILE | A | 325 | 24.842 | 16.758 | 40.609 | 1.00 | 39.38 | C |
| ATOM | 2037 | CD1 | ILE | A | 325 | 22.011 | 17.461 | 39.557 | 1.00 | 30.29 | C |
| ATOM | 2038 | N | TYR | A | 326 | 27.059 | 19.893 | 40.880 | 1.00 | 38.35 | N |
| ATOM | 2039 | CA | TYR | A | 326 | 28.513 | 19.866 | 40.748 | 1.00 | 42.65 | C |
| ATOM | 2040 | C | TYR | A | 326 | 29.206 | 20.323 | 42.021 | 1.00 | 54.64 | C |
| ATOM | 2041 | O | TYR | A | 326 | 30.425 | 20.207 | 42.152 | 1.00 | 53.74 | O |
| ATOM | 2042 | CB | TYR | A | 326 | 28.991 | 20.775 | 39.619 | 1.00 | 50.34 | C |
| ATOM | 2043 | CG | TYR | A | 326 | 28.528 | 20.386 | 38.247 | 1.00 | 47.66 | C |
| ATOM | 2044 | CD1 | TYR | A | 326 | 28.263 | 21.358 | 37.290 | 1.00 | 46.78 | C |
| ATOM | 2045 | CD2 | TYR | A | 326 | 28.347 | 19.057 | 37.904 | 1.00 | 49.11 | C |
| ATOM | 2046 | CE1 | TYR | A | 326 | 27.836 | 21.021 | 36.032 | 1.00 | 38.16 | C |
| ATOM | 2047 | CE2 | TYR | A | 326 | 27.914 | 18.705 | 36.641 | 1.00 | 45.70 | C |
| ATOM | 2048 | CZ | TYR | A | 326 | 27.660 | 19.693 | 35.714 | 1.00 | 42.81 | C |
| ATOM | 2049 | OH | TYR | A | 326 | 27.226 | 19.356 | 34.459 | 1.00 | 47.78 | O |
| ATOM | 2050 | N | CYS | A | 327 | 28.437 | 20.876 | 42.947 | 1.00 | 47.21 | N |
| ATOM | 2051 | CA | CYS | A | 327 | 29.007 | 21.312 | 44.205 | 1.00 | 40.99 | C |
| ATOM | 2052 | C | CYS | A | 327 | 29.341 | 20.101 | 45.068 | 1.00 | 49.85 | C |
| ATOM | 2053 | O | CYS | A | 327 | 30.042 | 20.216 | 46.071 | 1.00 | 57.22 | O |
| ATOM | 2054 | CB | CYS | A | 327 | 28.072 | 22.285 | 44.905 | 1.00 | 34.61 | C |
| ATOM | 2055 | SG | CYS | A | 327 | 27.994 | 23.895 | 44.076 | 1.00 | 56.73 | S |
| ATOM | 2056 | N | ARG | A | 328 | 28.859 | 18.936 | 44.641 | 1.00 | 46.95 | N |
| ATOM | 2057 | CA | ARG | A | 328 | 29.226 | 17.658 | 45.243 | 1.00 | 45.77 | C |
| ATOM | 2058 | C | ARG | A | 328 | 30.715 | 17.397 | 45.115 | 1.00 | 55.68 | C |
| ATOM | 2059 | O | ARG | A | 328 | 31.317 | 16.768 | 45.977 | 1.00 | 61.95 | O |
| ATOM | 2060 | CB | ARG | A | 328 | 28.486 | 16.521 | 44.558 | 1.00 | 43.92 | C |
| ATOM | 2061 | CG | ARG | A | 328 | 27.017 | 16.417 | 44.895 | 1.00 | 56.15 | C |
| ATOM | 2062 | CD | ARG | A | 328 | 26.452 | 15.285 | 44.095 | 1.00 | 47.45 | C |
| ATOM | 2063 | NE | ARG | A | 328 | 27.521 | 14.340 | 43.808 | 1.00 | 56.68 | N |
| ATOM | 2064 | CZ | ARG | A | 328 | 27.670 | 13.169 | 44.417 | 1.00 | 66.08 | C |
| ATOM | 2065 | NH1 | ARG | A | 328 | 26.794 | 12.784 | 45.337 | 1.00 | 65.47 | N |
| ATOM | 2066 | NH2 | ARG | A | 328 | 28.686 | 12.375 | 44.095 | 1.00 | 59.85 | N |
| ATOM | 2067 | N | SER | A | 329 | 31.301 | 17.855 | 44.016 | 1.00 | 64.43 | N |
| ATOM | 2068 | CA | SER | A | 329 | 32.741 | 17.761 | 43.826 | 1.00 | 62.35 | C |
| ATOM | 2069 | C | SER | A | 329 | 33.434 | 18.853 | 44.615 | 1.00 | 69.01 | C |
| ATOM | 2070 | O | SER | A | 329 | 32.899 | 19.949 | 44.768 | 1.00 | 66.26 | O |
| ATOM | 2071 | CB | SER | A | 329 | 33.108 | 17.906 | 42.353 | 1.00 | 69.85 | C |
| ATOM | 2072 | OG | SER | A | 329 | 34.434 | 18.390 | 42.217 | 1.00 | 67.34 | O |
| ATOM | 2073 | N | PRO | A | 330 | 34.640 | 18.557 | 45.114 | 1.00 | 84.00 | N |
| ATOM | 2074 | CA | PRO | A | 330 | 35.419 | 19.505 | 45.915 | 1.00 | 77.72 | C |
| ATOM | 2075 | C | PRO | A | 330 | 36.052 | 20.573 | 45.031 | 1.00 | 75.12 | C |
| ATOM | 2076 | O | PRO | A | 330 | 36.226 | 21.709 | 45.472 | 1.00 | 66.99 | O |
| ATOM | 2077 | CB | PRO | A | 330 | 36.509 | 18.625 | 46.544 | 1.00 | 81.03 | C |
| ATOM | 2078 | CG | PRO | A | 330 | 36.117 | 17.188 | 46.226 | 1.00 | 93.05 | C |
| ATOM | 2079 | CD | PRO | A | 330 | 35.329 | 17.266 | 44.968 | 1.00 | 88.46 | C |
| ATOM | 2080 | N | ASP | A | 331 | 36.381 | 20.201 | 43.795 | 1.00 | 77.68 | N |
| ATOM | 2081 | CA | ASP | A | 331 | 37.051 | 21.098 | 42.857 | 1.00 | 75.36 | C |
| ATOM | 2082 | C | ASP | A | 331 | 36.157 | 22.247 | 42.402 | 1.00 | 70.53 | C |
| ATOM | 2083 | O | ASP | A | 331 | 36.564 | 23.409 | 42.445 | 1.00 | 65.25 | O |
| ATOM | 2084 | CB | ASP | A | 331 | 37.537 | 20.321 | 41.634 | 1.00 | 88.89 | C |
| ATOM | 2085 | CG | ASP | A | 331 | 38.343 | 19.098 | 42.005 | 1.00 | 102.44 | C |
| ATOM | 2086 | OD1 | ASP | A | 331 | 38.749 | 18.992 | 43.183 | 1.00 | 99.44 | O |
| ATOM | 2087 | OD2 | ASP | A | 331 | 38.570 | 18.244 | 41.120 | 1.00 | 112.25 | O |
| ATOM | 2088 | N | PHE | A | 332 | 34.950 | 21.916 | 41.947 | 1.00 | 71.27 | N |
| ATOM | 2089 | CA | PHE | A | 332 | 33.987 | 22.929 | 41.523 | 1.00 | 69.05 | C |
| ATOM | 2090 | C | PHE | A | 332 | 33.677 | 23.857 | 42.689 | 1.00 | 71.72 | C |
| ATOM | 2091 | O | PHE | A | 332 | 33.635 | 25.079 | 42.534 | 1.00 | 64.41 | O |
| ATOM | 2092 | CB | PHE | A | 332 | 32.691 | 22.280 | 41.027 | 1.00 | 63.76 | C |
| ATOM | 2093 | CG | PHE | A | 332 | 32.760 | 21.774 | 39.609 | 1.00 | 66.85 | C |
| ATOM | 2094 | CD1 | PHE | A | 332 | 32.999 | 20.429 | 39.345 | 1.00 | 59.21 | C |
| ATOM | 2095 | CD2 | PHE | A | 332 | 32.567 | 22.642 | 38.536 | 1.00 | 66.38 | C |
| ATOM | 2096 | CE1 | PHE | A | 332 | 33.053 | 19.953 | 38.036 | 1.00 | 59.19 | C |
| ATOM | 2097 | CE2 | PHE | A | 332 | 32.626 | 22.175 | 37.223 | 1.00 | 60.53 | C |
| ATOM | 2098 | CZ | PHE | A | 332 | 32.868 | 20.824 | 36.974 | 1.00 | 52.06 | C |
| ATOM | 2099 | N | ARG | A | 333 | 33.464 | 23.252 | 43.855 | 1.00 | 71.60 | N |
| ATOM | 2100 | CA | ARG | A | 333 | 33.176 | 23.966 | 45.094 | 1.00 | 58.60 | C |
| ATOM | 2101 | C | ARG | A | 333 | 34.287 | 24.972 | 45.401 | 1.00 | 62.91 | C |
| ATOM | 2102 | O | ARG | A | 333 | 34.026 | 26.170 | 45.519 | 1.00 | 60.30 | O |
| ATOM | 2103 | CB | ARG | A | 333 | 33.053 | 22.952 | 46.228 | 1.00 | 66.66 | C |
| ATOM | 2104 | CG | ARG | A | 333 | 31.920 | 23.191 | 47.198 | 1.00 | 68.59 | C |
| ATOM | 2105 | CD | ARG | A | 333 | 31.584 | 21.890 | 47.920 | 1.00 | 76.56 | C |
| ATOM | 2106 | NE | ARG | A | 333 | 32.742 | 20.997 | 48.017 | 1.00 | 84.78 | N |
| ATOM | 2107 | CZ | ARG | A | 333 | 32.696 | 19.751 | 48.486 | 1.00 | 84.02 | C |
| ATOM | 2108 | NH1 | ARG | A | 333 | 31.547 | 19.244 | 48.910 | 1.00 | 78.43 | N |
| ATOM | 2109 | NH2 | ARG | A | 333 | 33.800 | 19.011 | 48.534 | 1.00 | 68.20 | N |
| ATOM | 2110 | N | LYS | A | 334 | 35.521 | 24.475 | 45.527 | 1.00 | 65.19 | N |
| ATOM | 2111 | CA | LYS | A | 334 | 36.696 | 25.327 | 45.717 | 1.00 | 68.09 | C |
| ATOM | 2112 | C | LYS | A | 334 | 36.688 | 26.471 | 44.710 | 1.00 | 70.85 | C |
| ATOM | 2113 | O | LYS | A | 334 | 36.854 | 27.642 | 45.069 | 1.00 | 62.94 | O |
| ATOM | 2114 | CB | LYS | A | 334 | 37.995 | 24.530 | 45.522 | 1.00 | 81.57 | C |
| ATOM | 2115 | CG | LYS | A | 334 | 38.332 | 23.473 | 46.576 | 1.00 | 83.57 | C |
| ATOM | 2116 | CD | LYS | A | 334 | 39.713 | 22.865 | 46.278 | 1.00 | 91.78 | C |
| ATOM | 2117 | CE | LYS | A | 334 | 39.839 | 21.418 | 46.761 | 1.00 | 93.06 | C |
| ATOM | 2118 | NZ | LYS | A | 334 | 40.917 | 20.659 | 46.041 | 1.00 | 61.77 | N |
| ATOM | 2119 | N | ALA | A | 335 | 36.499 | 26.107 | 43.443 | 1.00 | 69.41 | N |
| ATOM | 2120 | CA | ALA | A | 335 | 36.560 | 27.047 | 42.330 | 1.00 | 68.42 | C |
| ATOM | 2121 | C | ALA | A | 335 | 35.402 | 28.037 | 42.307 | 1.00 | 67.02 | C |
| ATOM | 2122 | O | ALA | A | 335 | 35.588 | 29.200 | 41.946 | 1.00 | 63.39 | O |
| ATOM | 2123 | CB | ALA | A | 335 | 36.638 | 26.297 | 41.014 | 1.00 | 69.49 | C |
| ATOM | 2124 | N | PHE | A | 336 | 34.209 | 27.576 | 42.672 | 1.00 | 68.84 | N |
| ATOM | 2125 | CA | PHE | A | 336 | 33.059 | 28.466 | 42.783 | 1.00 | 73.90 | C |
| ATOM | 2126 | C | PHE | A | 336 | 33.341 | 29.534 | 43.843 | 1.00 | 75.96 | C |
| ATOM | 2127 | O | PHE | A | 336 | 32.929 | 30.688 | 43.706 | 1.00 | 79.23 | O |
| ATOM | 2128 | CB | PHE | A | 336 | 31.784 | 27.698 | 43.160 | 1.00 | 74.10 | C |
| ATOM | 2129 | CG | PHE | A | 336 | 31.292 | 26.739 | 42.101 | 1.00 | 77.12 | C |
| ATOM | 2130 | CD1 | PHE | A | 336 | 31.368 | 27.058 | 40.754 | 1.00 | 71.25 | C |
| ATOM | 2131 | CD2 | PHE | A | 336 | 30.711 | 25.529 | 42.466 | 1.00 | 73.70 | C |
| ATOM | 2132 | CE1 | PHE | A | 336 | 30.903 | 26.175 | 39.788 | 1.00 | 64.33 | C |
| ATOM | 2133 | CE2 | PHE | A | 336 | 30.247 | 24.643 | 41.508 | 1.00 | 69.53 | C |
| ATOM | 2134 | CZ | PHE | A | 336 | 30.346 | 24.967 | 40.165 | 1.00 | 65.33 | C |
| ATOM | 2135 | N | LYS | A | 337 | 34.046 | 29.143 | 44.899 | 1.00 | 77.88 | N |
| ATOM | 2136 | CA | LYS | A | 337 | 34.337 | 30.055 | 46.005 | 1.00 | 82.32 | C |
| ATOM | 2137 | C | LYS | A | 337 | 35.461 | 31.046 | 45.671 | 1.00 | 81.10 | C |
| ATOM | 2138 | O | LYS | A | 337 | 35.337 | 32.240 | 45.948 | 1.00 | 77.24 | O |
| ATOM | 2139 | CB | LYS | A | 337 | 34.636 | 29.274 | 47.291 | 1.00 | 78.45 | C |
| ATOM | 2140 | CG | LYS | A | 337 | 33.492 | 28.361 | 47.726 | 1.00 | 82.00 | C |
| ATOM | 2141 | CD | LYS | A | 337 | 33.592 | 27.961 | 49.196 | 1.00 | 89.92 | C |
| ATOM | 2142 | CE | LYS | A | 337 | 32.410 | 27.086 | 49.612 | 1.00 | 89.55 | C |
| ATOM | 2143 | NZ | LYS | A | 337 | 32.424 | 26.743 | 51.064 | 1.00 | 84.38 | N |
| ATOM | 2144 | N | ARG | A | 338 | 36.552 | 30.556 | 45.085 | 1.00 | 71.18 | N |
| ATOM | 2145 | CA | ARG | A | 338 | 37.571 | 31.454 | 44.553 | 1.00 | 83.11 | C |
| ATOM | 2146 | C | ARG | A | 338 | 36.870 | 32.534 | 43.735 | 1.00 | 86.54 | C |
| ATOM | 2147 | O | ARG | A | 338 | 37.009 | 33.729 | 44.001 | 1.00 | 85.59 | O |
| ATOM | 2148 | CB | ARG | A | 338 | 38.542 | 30.711 | 43.632 | 1.00 | 86.26 | C |
| ATOM | 2149 | CG | ARG | A | 338 | 39.331 | 29.577 | 44.257 | 1.00 | 95.44 | C |
| ATOM | 2150 | CD | ARG | A | 338 | 40.254 | 28.965 | 43.205 | 1.00 | 106.82 | C |
| ATOM | 2151 | NE | ARG | A | 338 | 41.311 | 28.142 | 43.788 | 1.00 | 135.48 | N |
| ATOM | 2152 | CZ | ARG | A | 338 | 42.394 | 27.734 | 43.130 | 1.00 | 134.67 | C |
| ATOM | 2153 | NH1 | ARG | A | 338 | 42.575 | 28.073 | 41.858 | 1.00 | 122.13 | N |
| ATOM | 2154 | NH2 | ARG | A | 338 | 43.303 | 26.988 | 43.747 | 1.00 | 114.82 | N |
| ATOM | 2155 | N | LEU | A | 339 | 36.106 | 32.087 | 42.741 | 1.00 | 88.89 | N |
| ATOM | 2156 | CA | LEU | A | 339 | 35.394 | 32.963 | 41.811 | 1.00 | 84.99 | C |
| ATOM | 2157 | C | LEU | A | 339 | 34.468 | 33.969 | 42.491 | 1.00 | 88.07 | C |
| ATOM | 2158 | O | LEU | A | 339 | 34.228 | 35.055 | 41.963 | 1.00 | 85.96 | O |
| ATOM | 2159 | CB | LEU | A | 339 | 34.568 | 32.126 | 40.832 | 1.00 | 80.83 | C |
| ATOM | 2160 | CG | LEU | A | 339 | 35.278 | 31.243 | 39.805 | 1.00 | 90.75 | C |
| ATOM | 2161 | CD1 | LEU | A | 339 | 34.295 | 30.218 | 39.251 | 1.00 | 81.20 | C |
| ATOM | 2162 | CD2 | LEU | A | 339 | 35.892 | 32.074 | 38.677 | 1.00 | 76.22 | C |
| ATOM | 2163 | N | LEU | A | 340 | 33.930 | 33.602 | 43.648 | 1.00 | 86.03 | N |
| ATOM | 2164 | CA | LEU | A | 340 | 33.013 | 34.489 | 44.359 | 1.00 | 90.06 | C |
| ATOM | 2165 | C | LEU | A | 340 | 33.729 | 35.309 | 45.441 | 1.00 | 85.66 | C |
| ATOM | 2166 | O | LEU | A | 340 | 33.189 | 35.536 | 46.524 | 1.00 | 77.85 | O |
| ATOM | 2167 | CB | LEU | A | 340 | 31.837 | 33.692 | 44.933 | 1.00 | 85.58 | C |
| ATOM | 2168 | CG | LEU | A | 340 | 30.902 | 33.092 | 43.877 | 1.00 | 78.39 | C |
| ATOM | 2169 | CD1 | LEU | A | 340 | 30.206 | 31.825 | 44.372 | 1.00 | 80.26 | C |
| ATOM | 2170 | CD2 | LEU | A | 340 | 29.885 | 34.129 | 43.412 | 1.00 | 72.07 | C |
| ATOM | 2171 | N | ALA | A | 341 | 34.938 | 35.764 | 45.117 | 1.00 | 82.14 | N |
| ATOM | 2172 | CA | ALA | A | 341 | 35.721 | 36.624 | 45.998 | 1.00 | 73.03 | C |
| ATOM | 2173 | C | ALA | A | 341 | 35.692 | 36.132 | 47.444 | 1.00 | 106.96 | C |
| ATOM | 2174 | O | ALA | A | 341 | 35.595 | 36.925 | 48.383 | 1.00 | 120.03 | O |
| ATOM | 2175 | CB | ALA | A | 341 | 35.231 | 38.070 | 45.907 | 1.00 | 65.86 | C |
| ATOM | 2176 | C16 | PDL | A | 400 | 6.169 | 18.015 | 19.883 | 1.00 | 46.49 | C |
| ATOM | 2177 | N3 | PDL | A | 400 | 5.174 | 17.982 | 19.326 | 1.00 | 44.91 | N |
| ATOM | 2178 | N1 | PDL | A | 400 | 8.722 | 17.389 | 19.902 | 1.00 | 38.18 | N |
| ATOM | 2179 | C1 | PDL | A | 400 | 7.505 | 18.124 | 20.397 | 1.00 | 33.65 | C |
| ATOM | 2180 | C2 | PDL | A | 400 | 7.917 | 18.971 | 21.577 | 1.00 | 29.80 | C |
| ATOM | 2181 | C3 | PDL | A | 400 | 9.361 | 18.738 | 21.797 | 1.00 | 30.22 | C |
| ATOM | 2182 | C4 | PDL | A | 400 | 10.316 | 19.291 | 22.834 | 1.00 | 36.02 | C |
| ATOM | 2183 | C5 | PDL | A | 400 | 11.785 | 18.889 | 22.854 | 1.00 | 31.90 | C |
| ATOM | 2184 | C6 | PDL | A | 400 | 12.291 | 17.900 | 21.805 | 1.00 | 36.57 | C |
| ATOM | 2185 | C7 | PDL | A | 400 | 11.339 | 17.331 | 20.759 | 1.00 | 35.83 | C |
| ATOM | 2186 | C8 | PDL | A | 400 | 9.867 | 17.756 | 20.761 | 1.00 | 34.90 | C |
| ATOM | 2187 | O1 | PDL | A | 400 | 9.793 | 20.149 | 23.793 | 1.00 | 42.13 | O |
| ATOM | 2188 | C9 | PDL | A | 400 | 10.417 | 21.358 | 24.062 | 1.00 | 27.23 | C |
| ATOM | 2189 | C10 | PDL | A | 400 | 9.377 | 22.051 | 24.916 | 1.00 | 24.02 | C |
| ATOM | 2190 | O2 | PDL | A | 400 | 10.052 | 22.568 | 26.032 | 1.00 | 26.04 | O |
| ATOM | 2191 | C11 | PDL | A | 400 | 8.718 | 23.113 | 24.011 | 1.00 | 20.17 | C |
| ATOM | 2192 | N2 | PDL | A | 400 | 8.102 | 24.220 | 24.731 | 1.00 | 25.80 | N |
| ATOM | 2193 | C12 | PDL | A | 400 | 6.899 | 24.689 | 24.034 | 1.00 | 31.93 | C |
| ATOM | 2194 | C13 | PDL | A | 400 | 5.911 | 23.506 | 23.823 | 1.00 | 20.39 | C |
| ATOM | 2195 | C14 | PDL | A | 400 | 7.299 | 25.362 | 22.685 | 1.00 | 17.20 | C |
| ATOM | 2196 | C15 | PDL | A | 400 | 6.254 | 25.714 | 24.991 | 1.00 | 16.73 | C |
| ATOM | 2197 | NA | NA | A | 401 | 0.643 | 32.135 | 15.873 | 1.00 | 36.22 | Na |

TABLE B

| | | | | | | | |
|---|---|---|---|---|---|---|---|
| CRYST1 | 55.500 | 86.800 | 95.500 | 67.60 | 73.30 | 85.80 | P1 |

| | | | | |
|---|---|---|---|---|
| SCALE1 | 0.018018 | −0.001323 | −0.005298 | 0.00000 |
| SCALE2 | 0.000000 | 0.011552 | −0.004700 | 0.00000 |
| SCALE3 | 0.000000 | 0.000000 | 0.011803 | 0.00000 |

| | | | | | | | | | | | |
|---|---|---|---|---|---|---|---|---|---|---|---|
| ATOM | 2198 | N | GLN | B | 31 | 36.149 | −5.203 | −24.403 | 1.00 | 81.77 | N |
| ATOM | 2199 | CA | GLN | B | 31 | 34.722 | −5.513 | −24.547 | 1.00 | 89.90 | C |
| ATOM | 2200 | C | GLN | B | 31 | 34.186 | −6.397 | −23.410 | 1.00 | 84.77 | C |
| ATOM | 2201 | O | GLN | B | 31 | 33.071 | −6.915 | −23.475 | 1.00 | 90.50 | O |
| ATOM | 2202 | CB | GLN | B | 31 | 34.431 | −6.163 | −25.902 | 1.00 | 90.00 | C |
| ATOM | 2203 | CG | GLN | B | 31 | 33.264 | −5.532 | −26.640 | 1.00 | 80.74 | C |
| ATOM | 2204 | CD | GLN | B | 31 | 33.722 | −4.445 | −27.585 | 1.00 | 69.73 | C |
| ATOM | 2205 | OE1 | GLN | B | 31 | 34.894 | −4.072 | −27.590 | 1.00 | 70.46 | O |
| ATOM | 2206 | NE2 | GLN | B | 31 | 32.808 | −3.948 | −28.408 | 1.00 | 58.43 | N |
| ATOM | 2207 | N | TRP | B | 32 | 35.014 | −6.597 | −22.395 | 1.00 | 82.72 | N |
| ATOM | 2208 | CA | TRP | B | 32 | 34.565 | −6.977 | −21.065 | 1.00 | 65.66 | C |
| ATOM | 2209 | C | TRP | B | 32 | 33.421 | −6.044 | −20.645 | 1.00 | 72.87 | C |
| ATOM | 2210 | O | TRP | B | 32 | 32.620 | −6.382 | −19.776 | 1.00 | 77.95 | O |
| ATOM | 2211 | CB | TRP | B | 32 | 35.753 | −6.878 | −20.107 | 1.00 | 59.16 | C |
| ATOM | 2212 | CG | TRP | B | 32 | 35.424 | −6.874 | −18.657 | 1.00 | 81.99 | C |
| ATOM | 2213 | CD1 | TRP | B | 32 | 35.362 | −7.958 | −17.828 | 1.00 | 90.78 | C |
| ATOM | 2214 | CD2 | TRP | B | 32 | 35.149 | −5.724 | −17.841 | 1.00 | 83.95 | C |
| ATOM | 2215 | NE1 | TRP | B | 32 | 35.049 | −7.556 | −16.549 | 1.00 | 100.38 | N |
| ATOM | 2216 | CE2 | TRP | B | 32 | 34.912 | −6.192 | −16.529 | 1.00 | 96.28 | C |
| ATOM | 2217 | CE3 | TRP | B | 32 | 35.066 | −4.350 | −18.094 | 1.00 | 67.70 | C |
| ATOM | 2218 | CZ2 | TRP | B | 32 | 34.597 | −5.328 | −15.470 | 1.00 | 77.74 | C |
| ATOM | 2219 | CZ3 | TRP | B | 32 | 34.754 | −3.494 | −17.042 | 1.00 | 64.94 | C |
| ATOM | 2220 | CH2 | TRP | B | 32 | 34.524 | −3.988 | −15.748 | 1.00 | 65.52 | C |
| ATOM | 2221 | N | GLU | B | 33 | 33.341 | −4.877 | −21.285 | 1.00 | 72.51 | N |
| ATOM | 2222 | CA | GLU | B | 33 | 32.250 | −3.927 | −21.059 | 1.00 | 58.20 | C |
| ATOM | 2223 | C | GLU | B | 33 | 30.899 | −4.501 | −21.460 | 1.00 | 57.23 | C |
| ATOM | 2224 | O | GLU | B | 33 | 29.879 | −4.144 | −20.883 | 1.00 | 60.36 | O |
| ATOM | 2225 | CB | GLU | B | 33 | 32.481 | −2.625 | −21.838 | 1.00 | 59.22 | C |
| ATOM | 2226 | CG | GLU | B | 33 | 31.228 | −1.736 | −21.943 | 1.00 | 65.68 | C |
| ATOM | 2227 | CD | GLU | B | 33 | 31.378 | −0.539 | −22.895 | 1.00 | 78.55 | C |
| ATOM | 2228 | OE1 | GLU | B | 33 | 32.441 | −0.400 | −23.543 | 1.00 | 76.32 | O |
| ATOM | 2229 | OE2 | GLU | B | 33 | 30.424 | 0.271 | −22.993 | 1.00 | 60.84 | O |
| ATOM | 2230 | N | ALA | B | 34 | 30.891 | −5.378 | −22.459 | 1.00 | 68.27 | N |
| ATOM | 2231 | CA | ALA | B | 34 | 29.642 | −5.896 | −23.014 | 1.00 | 65.86 | C |
| ATOM | 2232 | C | ALA | B | 34 | 29.013 | −6.991 | −22.155 | 1.00 | 67.93 | C |
| ATOM | 2233 | O | ALA | B | 34 | 27.793 | −7.026 | −21.990 | 1.00 | 64.00 | O |
| ATOM | 2234 | CB | ALA | B | 34 | 29.856 | −6.389 | −24.432 | 1.00 | 63.85 | C |
| ATOM | 2235 | N | GLY | B | 35 | 29.842 | −7.882 | −21.616 | 1.00 | 62.49 | N |
| ATOM | 2236 | CA | GLY | B | 35 | 29.356 | −8.930 | −20.738 | 1.00 | 48.88 | C |
| ATOM | 2237 | C | GLY | B | 35 | 28.877 | −8.348 | −19.421 | 1.00 | 60.74 | C |
| ATOM | 2238 | O | GLY | B | 35 | 27.940 | −8.851 | −18.798 | 1.00 | 62.64 | O |
| ATOM | 2239 | N | MET | B | 36 | 29.528 | −7.270 | −19.001 | 1.00 | 59.62 | N |
| ATOM | 2240 | CA | MET | B | 36 | 29.181 | −6.589 | −17.765 | 1.00 | 51.03 | C |
| ATOM | 2241 | C | MET | B | 36 | 27.827 | −5.883 | −17.916 | 1.00 | 60.69 | C |
| ATOM | 2242 | O | MET | B | 36 | 26.979 | −5.959 | −17.030 | 1.00 | 65.75 | O |
| ATOM | 2243 | CB | MET | B | 36 | 30.289 | −5.605 | −17.389 | 1.00 | 53.08 | C |
| ATOM | 2244 | CG | MET | B | 36 | 30.521 | −5.432 | −15.892 | 1.00 | 79.54 | C |
| ATOM | 2245 | SD | MET | B | 36 | 30.994 | −6.941 | −15.011 | 1.00 | 69.83 | S |
| ATOM | 2246 | CE | MET | B | 36 | 32.036 | −7.739 | −16.225 | 1.00 | 77.15 | C |
| ATOM | 2247 | N | SER | B | 37 | 27.616 | −5.219 | −19.050 | 1.00 | 60.19 | N |
| ATOM | 2248 | CA | SER | B | 37 | 26.336 | −4.575 | −19.339 | 1.00 | 49.26 | C |
| ATOM | 2249 | C | SER | B | 37 | 25.237 | −5.614 | −19.537 | 1.00 | 56.99 | C |
| ATOM | 2250 | O | SER | B | 37 | 24.068 | −5.272 | −19.715 | 1.00 | 51.07 | O |
| ATOM | 2251 | CB | SER | B | 37 | 26.434 | −3.717 | −20.602 | 1.00 | 53.61 | C |
| ATOM | 2252 | OG | SER | B | 37 | 27.490 | −2.774 | −20.529 | 1.00 | 58.61 | O |
| ATOM | 2253 | N | LEU | B | 38 | 25.618 | −6.886 | −19.529 | 1.00 | 63.20 | N |
| ATOM | 2254 | CA | LEU | B | 38 | 24.645 | −7.951 | −19.681 | 1.00 | 58.95 | C |
| ATOM | 2255 | C | LEU | B | 38 | 24.163 | −8.439 | −18.317 | 1.00 | 58.92 | C |
| ATOM | 2256 | O | LEU | B | 38 | 22.963 | −8.445 | −18.051 | 1.00 | 52.06 | O |
| ATOM | 2257 | CB | LEU | B | 38 | 25.216 | −9.103 | −20.495 | 1.00 | 60.62 | C |
| ATOM | 2258 | CG | LEU | B | 38 | 24.150 | −9.871 | −21.273 | 1.00 | 75.65 | C |
| ATOM | 2259 | CD1 | LEU | B | 38 | 23.705 | −9.065 | −22.484 | 1.00 | 61.05 | C |
| ATOM | 2260 | CD2 | LEU | B | 38 | 24.676 | −11.223 | −21.692 | 1.00 | 76.10 | C |
| ATOM | 2261 | N | LEU | B | 39 | 25.093 | −8.840 | −17.450 | 1.00 | 58.52 | N |
| ATOM | 2262 | CA | LEU | B | 39 | 24.718 | −9.254 | −16.094 | 1.00 | 74.76 | C |
| ATOM | 2263 | C | LEU | B | 39 | 24.095 | −8.090 | −15.330 | 1.00 | 63.36 | C |
| ATOM | 2264 | O | LEU | B | 39 | 23.247 | −8.282 | −14.456 | 1.00 | 59.24 | O |
| ATOM | 2265 | CB | LEU | B | 39 | 25.902 | −9.839 | −15.301 | 1.00 | 74.03 | C |
| ATOM | 2266 | CG | LEU | B | 39 | 26.230 | −11.335 | −15.454 | 1.00 | 93.97 | C |
| ATOM | 2267 | CD1 | LEU | B | 39 | 26.624 | −11.950 | −14.105 | 1.00 | 76.81 | C |
| ATOM | 2268 | CD2 | LEU | B | 39 | 25.070 | −12.124 | −16.066 | 1.00 | 74.37 | C |
| ATOM | 2269 | N | MET | B | 40 | 24.515 | −6.878 | −15.667 | 1.00 | 62.09 | N |
| ATOM | 2270 | CA | MET | B | 40 | 23.966 | −5.708 | −15.011 | 1.00 | 56.98 | C |
| ATOM | 2271 | C | MET | B | 40 | 22.535 | −5.511 | −15.493 | 1.00 | 49.68 | C |
| ATOM | 2272 | O | MET | B | 40 | 21.609 | −5.408 | −14.694 | 1.00 | 52.50 | O |
| ATOM | 2273 | CB | MET | B | 40 | 24.824 | −4.474 | −15.288 | 1.00 | 46.31 | C |
| ATOM | 2274 | CG | MET | B | 40 | 25.127 | −3.644 | −14.037 | 1.00 | 51.47 | C |
| ATOM | 2275 | SD | MET | B | 40 | 26.046 | −4.512 | −12.733 | 1.00 | 71.14 | S |
| ATOM | 2276 | CE | MET | B | 40 | 27.694 | −4.565 | −13.450 | 1.00 | 77.35 | C |
| ATOM | 2277 | N | ALA | B | 41 | 22.353 | −5.490 | −16.805 | 1.00 | 39.02 | N |
| ATOM | 2278 | CA | ALA | B | 41 | 21.021 | −5.398 | −17.373 | 1.00 | 42.96 | C |
| ATOM | 2279 | C | ALA | B | 41 | 20.099 | −6.479 | −16.801 | 1.00 | 48.24 | C |
| ATOM | 2280 | O | ALA | B | 41 | 18.884 | −6.296 | −16.718 | 1.00 | 39.23 | O |
| ATOM | 2281 | CB | ALA | B | 41 | 21.094 | −5.514 | −18.875 | 1.00 | 36.52 | C |
| ATOM | 2282 | N | LEU | B | 42 | 20.689 | −7.602 | −16.405 | 1.00 | 52.52 | N |
| ATOM | 2283 | CA | LEU | B | 42 | 19.932 | −8.733 | −15.884 | 1.00 | 51.79 | C |
| ATOM | 2284 | C | LEU | B | 42 | 19.400 | −8.463 | −14.483 | 1.00 | 52.03 | C |
| ATOM | 2285 | O | LEU | B | 42 | 18.228 | −8.713 | −14.207 | 1.00 | 49.97 | O |
| ATOM | 2286 | CB | LEU | B | 42 | 20.802 | −9.986 | −15.852 | 1.00 | 59.33 | C |
| ATOM | 2287 | CG | LEU | B | 42 | 20.035 | −11.300 | −15.956 | 1.00 | 66.47 | C |
| ATOM | 2288 | CD1 | LEU | B | 42 | 19.940 | −11.694 | −17.421 | 1.00 | 52.52 | C |
| ATOM | 2289 | CD2 | LEU | B | 42 | 20.708 | −12.393 | −15.137 | 1.00 | 59.25 | C |
| ATOM | 2290 | N | VAL | B | 43 | 20.266 | −7.974 | −13.597 | 1.00 | 47.11 | N |
| ATOM | 2291 | CA | VAL | B | 43 | 19.849 | −7.648 | −12.235 | 1.00 | 43.68 | C |
| ATOM | 2292 | C | VAL | B | 43 | 18.769 | −6.574 | −12.246 | 1.00 | 41.74 | C |
| ATOM | 2293 | O | VAL | B | 43 | 17.761 | −6.706 | −11.566 | 1.00 | 48.21 | O |
| ATOM | 2294 | CB | VAL | B | 43 | 21.024 | −7.209 | −11.325 | 1.00 | 41.47 | C |
| ATOM | 2295 | CG1 | VAL | B | 43 | 22.078 | −8.291 | −11.268 | 1.00 | 46.55 | C |
| ATOM | 2296 | CG2 | VAL | B | 43 | 21.632 | −5.917 | −11.813 | 1.00 | 47.55 | C |
| ATOM | 2297 | N | VAL | B | 44 | 18.969 | −5.520 | −13.030 | 1.00 | 34.14 | N |
| ATOM | 2298 | CA | VAL | B | 44 | 17.959 | −4.483 | −13.159 | 1.00 | 35.83 | C |
| ATOM | 2299 | C | VAL | B | 44 | 16.606 | −5.108 | −13.514 | 1.00 | 44.98 | C |
| ATOM | 2300 | O | VAL | B | 44 | 15.549 | −4.656 | −13.062 | 1.00 | 42.32 | O |
| ATOM | 2301 | CB | VAL | B | 44 | 18.353 | −3.446 | −14.220 | 1.00 | 28.33 | C |
| ATOM | 2302 | CG1 | VAL | B | 44 | 17.180 | −2.524 | −14.545 | 1.00 | 20.72 | C |
| ATOM | 2303 | CG2 | VAL | B | 44 | 19.566 | −2.652 | −13.748 | 1.00 | 31.64 | C |
| ATOM | 2304 | N | LEU | B | 45 | 16.655 | −6.171 | −14.307 | 1.00 | 49.30 | N |
| ATOM | 2305 | CA | LEU | B | 45 | 15.453 | −6.889 | −14.717 | 1.00 | 52.52 | C |
| ATOM | 2306 | C | LEU | B | 45 | 14.790 | −7.645 | −13.563 | 1.00 | 45.52 | C |
| ATOM | 2307 | O | LEU | B | 45 | 13.577 | −7.562 | −13.379 | 1.00 | 41.75 | O |
| ATOM | 2308 | CB | LEU | B | 45 | 15.782 | −7.870 | −15.838 | 1.00 | 54.51 | C |
| ATOM | 2309 | CG | LEU | B | 45 | 14.581 | −8.718 | −16.251 | 1.00 | 56.80 | C |
| ATOM | 2310 | CD1 | LEU | B | 45 | 13.548 | −7.835 | −16.926 | 1.00 | 49.11 | C |
| ATOM | 2311 | CD2 | LEU | B | 45 | 14.998 | −9.860 | −17.154 | 1.00 | 43.93 | C |
| ATOM | 2312 | N | LEU | B | 46 | 15.588 | −8.409 | −12.820 | 1.00 | 39.24 | N |
| ATOM | 2313 | CA | LEU | B | 46 | 15.126 | −9.060 | −11.602 | 1.00 | 43.79 | C |
| ATOM | 2314 | C | LEU | B | 46 | 14.451 | −8.056 | −10.673 | 1.00 | 50.22 | C |
| ATOM | 2315 | O | LEU | B | 46 | 13.233 | −8.078 | −10.503 | 1.00 | 51.19 | O |
| ATOM | 2316 | CB | LEU | B | 46 | 16.301 | −9.701 | −10.871 | 1.00 | 44.13 | C |
| ATOM | 2317 | CG | LEU | B | 46 | 16.563 | −11.165 | −11.175 | 1.00 | 50.55 | C |
| ATOM | 2318 | CD1 | LEU | B | 46 | 17.647 | −11.707 | −10.251 | 1.00 | 44.57 | C |
| ATOM | 2319 | CD2 | LEU | B | 46 | 15.267 | −11.939 | −10.998 | 1.00 | 60.03 | C |
| ATOM | 2320 | N | ILE | B | 47 | 15.258 | −7.177 | −10.080 | 1.00 | 43.53 | N |
| ATOM | 2321 | CA | ILE | B | 47 | 14.767 | −6.119 | −9.204 | 1.00 | 48.12 | C |
| ATOM | 2322 | C | ILE | B | 47 | 13.527 | −5.417 | −9.741 | 1.00 | 41.51 | C |
| ATOM | 2323 | O | ILE | B | 47 | 12.555 | −5.240 | −9.011 | 1.00 | 42.93 | O |
| ATOM | 2324 | CB | ILE | B | 47 | 15.843 | −5.036 | −8.940 | 1.00 | 49.99 | C |
| ATOM | 2325 | CG1 | ILE | B | 47 | 17.100 | −5.653 | −8.337 | 1.00 | 39.53 | C |
| ATOM | 2326 | CG2 | ILE | B | 47 | 15.296 | −3.939 | −8.023 | 1.00 | 34.66 | C |
| ATOM | 2327 | CD1 | ILE | B | 47 | 18.296 | −4.738 | −8.403 | 1.00 | 35.38 | C |
| ATOM | 2328 | N | VAL | B | 48 | 13.554 | −4.994 | −11.001 | 1.00 | 33.39 | N |
| ATOM | 2329 | CA | VAL | B | 48 | 12.411 | −4.241 | −11.505 | 1.00 | 40.39 | C |
| ATOM | 2330 | C | VAL | B | 48 | 11.171 | −5.097 | −11.699 | 1.00 | 45.70 | C |
| ATOM | 2331 | O | VAL | B | 48 | 10.068 | −4.688 | −11.331 | 1.00 | 57.27 | O |
| ATOM | 2332 | CB | VAL | B | 48 | 12.701 | −3.460 | −12.783 | 1.00 | 36.27 | C |
| ATOM | 2333 | CG1 | VAL | B | 48 | 11.399 | −2.885 | −13.322 | 1.00 | 28.81 | C |
| ATOM | 2334 | CG2 | VAL | B | 48 | 13.698 | −2.333 | −12.503 | 1.00 | 34.41 | C |
| ATOM | 2335 | N | ALA | B | 49 | 11.343 | −6.286 | −12.262 | 1.00 | 44.35 | N |
| ATOM | 2336 | CA | ALA | B | 49 | 10.201 | −7.168 | −12.496 | 1.00 | 49.26 | C |
| ATOM | 2337 | C | ALA | B | 49 | 9.562 | −7.669 | −11.190 | 1.00 | 50.96 | C |
| ATOM | 2338 | O | ALA | B | 49 | 8.342 | −7.591 | −11.005 | 1.00 | 43.23 | O |
| ATOM | 2339 | CB | ALA | B | 49 | 10.599 | −8.341 | −13.385 | 1.00 | 32.52 | C |
| ATOM | 2340 | N | GLY | B | 50 | 10.391 | −8.181 | −10.287 | 1.00 | 44.01 | N |
| ATOM | 2341 | CA | GLY | B | 50 | 9.899 | −8.779 | −9.060 | 1.00 | 48.90 | C |
| ATOM | 2342 | C | GLY | B | 50 | 9.195 | −7.794 | −8.145 | 1.00 | 49.18 | C |
| ATOM | 2343 | O | GLY | B | 50 | 8.241 | −8.138 | −7.453 | 1.00 | 48.56 | O |
| ATOM | 2344 | N | ASN | B | 51 | 9.665 | −6.557 | −8.140 | 1.00 | 47.93 | N |
| ATOM | 2345 | CA | ASN | B | 51 | 9.116 | −5.569 | −7.235 | 1.00 | 44.33 | C |
| ATOM | 2346 | C | ASN | B | 51 | 7.899 | −4.894 | −7.819 | 1.00 | 47.36 | C |
| ATOM | 2347 | O | ASN | B | 51 | 7.081 | −4.343 | −7.088 | 1.00 | 55.88 | O |
| ATOM | 2348 | CB | ASN | B | 51 | 10.173 | −4.538 | −6.857 | 1.00 | 44.74 | C |
| ATOM | 2349 | CG | ASN | B | 51 | 11.077 | −5.026 | −5.751 | 1.00 | 48.93 | C |
| ATOM | 2350 | OD1 | ASN | B | 51 | 10.668 | −5.095 | −4.586 | 1.00 | 49.15 | O |
| ATOM | 2351 | ND2 | ASN | B | 51 | 12.315 | −5.376 | −6.104 | 1.00 | 44.73 | N |
| ATOM | 2352 | N | VAL | B | 52 | 7.785 | −4.926 | −9.139 | 1.00 | 41.94 | N |
| ATOM | 2353 | CA | VAL | B | 52 | 6.594 | −4.403 | −9.787 | 1.00 | 46.77 | C |
| ATOM | 2354 | C | VAL | B | 52 | 5.500 | −5.455 | −9.644 | 1.00 | 49.35 | C |
| ATOM | 2355 | O | VAL | B | 52 | 4.314 | −5.137 | −9.563 | 1.00 | 46.91 | O |
| ATOM | 2356 | CB | VAL | B | 52 | 6.865 | −4.064 | −11.263 | 1.00 | 38.84 | C |
| ATOM | 2357 | CG1 | VAL | B | 52 | 5.571 | −3.939 | −12.036 | 1.00 | 22.72 | C |
| ATOM | 2358 | CG2 | VAL | B | 52 | 7.669 | −2.780 | −11.355 | 1.00 | 38.08 | C |
| ATOM | 2359 | N | LEU | B | 53 | 5.932 | −6.710 | −9.573 | 1.00 | 48.26 | N |
| ATOM | 2360 | CA | LEU | B | 53 | 5.049 | −7.850 | −9.376 | 1.00 | 46.69 | C |
| ATOM | 2361 | C | LEU | B | 53 | 4.378 | −7.776 | −8.012 | 1.00 | 56.49 | C |
| ATOM | 2362 | O | LEU | B | 53 | 3.160 | −7.915 | −7.891 | 1.00 | 55.54 | O |
| ATOM | 2363 | CB | LEU | B | 53 | 5.865 | −9.135 | −9.467 | 1.00 | 55.12 | C |
| ATOM | 2364 | CG | LEU | B | 53 | 5.262 | −10.279 | −10.273 | 1.00 | 68.12 | C |
| ATOM | 2365 | CD1 | LEU | B | 53 | 4.778 | −9.773 | −11.622 | 1.00 | 55.24 | C |
| ATOM | 2366 | CD2 | LEU | B | 53 | 6.293 | −11.376 | −10.456 | 1.00 | 79.59 | C |
| ATOM | 2367 | N | VAL | B | 54 | 5.192 | −7.568 | −6.982 | 1.00 | 61.13 | N |
| ATOM | 2368 | CA | VAL | B | 54 | 4.704 | −7.394 | −5.621 | 1.00 | 49.52 | C |
| ATOM | 2369 | C | VAL | B | 54 | 3.686 | −6.258 | −5.530 | 1.00 | 50.76 | C |
| ATOM | 2370 | O | VAL | B | 54 | 2.550 | −6.470 | −5.118 | 1.00 | 51.56 | O |
| ATOM | 2371 | CB | VAL | B | 54 | 5.865 | −7.113 | −4.655 | 1.00 | 44.36 | C |
| ATOM | 2372 | CG1 | VAL | B | 54 | 5.337 | −6.715 | −3.283 | 1.00 | 49.18 | C |
| ATOM | 2373 | CG2 | VAL | B | 54 | 6.772 | −8.329 | −4.554 | 1.00 | 49.20 | C |
| ATOM | 2374 | N | ILE | B | 55 | 4.096 | −5.057 | −5.923 | 1.00 | 48.29 | N |
| ATOM | 2375 | CA | ILE | B | 55 | 3.226 | −3.889 | −5.844 | 1.00 | 48.41 | C |
| ATOM | 2376 | C | ILE | B | 55 | 1.913 | −4.111 | −6.581 | 1.00 | 55.18 | C |
| ATOM | 2377 | O | ILE | B | 55 | 0.859 | −3.678 | −6.121 | 1.00 | 68.61 | O |
| ATOM | 2378 | CB | ILE | B | 55 | 3.898 | −2.630 | −6.410 | 1.00 | 45.84 | C |
| ATOM | 2379 | CG1 | ILE | B | 55 | 5.025 | −2.164 | −5.487 | 1.00 | 45.38 | C |
| ATOM | 2380 | CG2 | ILE | B | 55 | 2.878 | −1.528 | −6.576 | 1.00 | 38.25 | C |
| ATOM | 2381 | CD1 | ILE | B | 55 | 6.055 | −1.278 | −6.175 | 1.00 | 39.92 | C |
| ATOM | 2382 | N | ALA | B | 56 | 1.969 | −4.790 | −7.719 | 1.00 | 50.12 | N |
| ATOM | 2383 | CA | ALA | B | 56 | 0.759 | −5.044 | −8.487 | 1.00 | 49.96 | C |
| ATOM | 2384 | C | ALA | B | 56 | −0.101 | −6.137 | −7.859 | 1.00 | 55.20 | C |
| ATOM | 2385 | O | ALA | B | 56 | −1.322 | −6.021 | −7.828 | 1.00 | 60.94 | O |
| ATOM | 2386 | CB | ALA | B | 56 | 1.101 | −5.391 | −9.915 | 1.00 | 38.35 | C |
| ATOM | 2387 | N | ALA | B | 57 | 0.535 | −7.193 | −7.358 | 1.00 | 52.20 | N |
| ATOM | 2388 | CA | ALA | B | 57 | −0.190 | −8.296 | −6.726 | 1.00 | 64.35 | C |
| ATOM | 2389 | C | ALA | B | 57 | −1.006 | −7.817 | −5.527 | 1.00 | 74.92 | C |
| ATOM | 2390 | O | ALA | B | 57 | −2.169 | −8.192 | −5.359 | 1.00 | 78.24 | O |
| ATOM | 2391 | CB | ALA | B | 57 | 0.771 | −9.394 | −6.299 | 1.00 | 63.84 | C |
| ATOM | 2392 | N | ILE | B | 58 | −0.381 | −6.995 | −4.691 | 1.00 | 72.21 | N |
| ATOM | 2393 | CA | ILE | B | 58 | −1.051 | −6.424 | −3.533 | 1.00 | 68.16 | C |
| ATOM | 2394 | C | ILE | B | 58 | −2.139 | −5.470 | −4.001 | 1.00 | 66.75 | C |
| ATOM | 2395 | O | ILE | B | 58 | −3.223 | −5.423 | −3.424 | 1.00 | 81.83 | O |
| ATOM | 2396 | CB | ILE | B | 58 | −0.048 | −5.706 | −2.607 | 1.00 | 59.43 | C |
| ATOM | 2397 | CG1 | ILE | B | 58 | 0.888 | −6.728 | −1.969 | 1.00 | 50.22 | C |
| ATOM | 2398 | CG2 | ILE | B | 58 | −0.763 | −4.914 | −1.519 | 1.00 | 50.40 | C |
| ATOM | 2399 | CD1 | ILE | B | 58 | 2.031 | −6.101 | −1.210 | 1.00 | 60.36 | C |
| ATOM | 2400 | N | GLY | B | 59 | −1.851 | −4.726 | −5.063 | 1.00 | 62.24 | N |
| ATOM | 2401 | CA | GLY | B | 59 | −2.830 | −3.825 | −5.643 | 1.00 | 81.36 | C |
| ATOM | 2402 | C | GLY | B | 59 | −4.028 | −4.546 | −6.243 | 1.00 | 88.64 | C |
| ATOM | 2403 | O | GLY | B | 59 | −5.129 | −3.996 | −6.306 | 1.00 | 81.11 | O |
| ATOM | 2404 | N | SER | B | 60 | −3.812 | −5.783 | −6.682 | 1.00 | 88.58 | N |
| ATOM | 2405 | CA | SER | B | 60 | −4.862 | −6.565 | −7.332 | 1.00 | 93.29 | C |
| ATOM | 2406 | C | SER | B | 60 | −5.826 | −7.177 | −6.319 | 1.00 | 91.37 | C |
| ATOM | 2407 | O | SER | B | 60 | −6.997 | −6.804 | −6.263 | 1.00 | 110.99 | O |
| ATOM | 2408 | CB | SER | B | 60 | −4.257 | −7.657 | −8.223 | 1.00 | 72.53 | C |
| ATOM | 2409 | OG | SER | B | 60 | −3.527 | −7.086 | −9.295 | 1.00 | 70.64 | O |
| ATOM | 2410 | N | THR | B | 61 | −5.332 | −8.119 | −5.523 | 1.00 | 86.94 | N |
| ATOM | 2411 | CA | THR | B | 61 | −6.149 | −8.765 | −4.500 | 1.00 | 102.13 | C |
| ATOM | 2412 | C | THR | B | 61 | −6.273 | −7.879 | −3.262 | 1.00 | 113.92 | C |
| ATOM | 2413 | O | THR | B | 61 | −5.268 | −7.375 | −2.752 | 1.00 | 113.13 | O |
| ATOM | 2414 | CB | THR | B | 61 | −5.529 | −10.107 | −4.057 | 1.00 | 107.16 | C |
| ATOM | 2415 | OG1 | THR | B | 61 | −4.709 | −10.637 | −5.108 | 1.00 | 103.36 | O |
| ATOM | 2416 | CG2 | THR | B | 61 | −6.619 | −11.112 | −3.682 | 1.00 | 104.62 | C |
| ATOM | 2417 | N | GLN | B | 62 | −7.497 | −7.686 | −2.777 | 1.00 | 111.63 | N |
| ATOM | 2418 | CA | GLN | B | 62 | −7.688 | −6.999 | −1.502 | 1.00 | 113.97 | C |
| ATOM | 2419 | C | GLN | B | 62 | −7.446 | −8.003 | −0.387 | 1.00 | 106.34 | C |
| ATOM | 2420 | O | GLN | B | 62 | −7.163 | −7.631 | 0.753 | 1.00 | 91.97 | O |
| ATOM | 2421 | CB | GLN | B | 62 | −9.099 | −6.439 | −1.380 | 1.00 | 118.58 | C |
| ATOM | 2422 | CG | GLN | B | 62 | −9.664 | −5.890 | −2.665 | 1.00 | 121.46 | C |
| ATOM | 2423 | CD | GLN | B | 62 | −11.155 | −6.117 | −2.754 | 1.00 | 101.14 | C |
| ATOM | 2424 | OE1 | GLN | B | 62 | −11.777 | −6.579 | −1.794 | 1.00 | 84.46 | O |
| ATOM | 2425 | NE2 | GLN | B | 62 | −11.739 | −5.807 | −3.908 | 1.00 | 95.67 | N |
| ATOM | 2426 | N | ARG | B | 63 | −7.578 | −9.282 | −0.729 | 1.00 | 102.88 | N |
| ATOM | 2427 | CA | ARG | B | 63 | −7.207 | −10.353 | 0.177 | 1.00 | 97.41 | C |
| ATOM | 2428 | C | ARG | B | 63 | −5.760 | −10.139 | 0.611 | 1.00 | 104.74 | C |
| ATOM | 2429 | O | ARG | B | 63 | −5.431 | −10.213 | 1.798 | 1.00 | 90.21 | O |
| ATOM | 2430 | CB | ARG | B | 63 | −7.352 | −11.705 | −0.511 | 1.00 | 85.82 | C |
| ATOM | 2431 | CG | ARG | B | 63 | −7.031 | −12.868 | 0.396 | 1.00 | 107.12 | C |
| ATOM | 2432 | CD | ARG | B | 63 | −6.779 | −14.136 | −0.385 | 1.00 | 113.27 | C |
| ATOM | 2433 | NE | ARG | B | 63 | −6.369 | −15.220 | 0.502 | 1.00 | 131.42 | N |
| ATOM | 2434 | CZ | ARG | B | 63 | −6.090 | −16.455 | 0.098 | 1.00 | 143.25 | C |
| ATOM | 2435 | NH1 | ARG | B | 63 | −6.172 | −16.767 | −1.188 | 1.00 | 148.48 | N |
| ATOM | 2436 | NH2 | ARG | B | 63 | −5.722 | −17.377 | 0.979 | 1.00 | 137.33 | N |
| ATOM | 2437 | N | LEU | B | 64 | −4.901 | −9.861 | −0.366 | 1.00 | 106.61 | N |
| ATOM | 2438 | CA | LEU | B | 64 | −3.511 | −9.513 | −0.099 | 1.00 | 94.75 | C |
| ATOM | 2439 | C | LEU | B | 64 | −3.369 | −8.114 | 0.519 | 1.00 | 89.75 | C |
| ATOM | 2440 | O | LEU | B | 64 | −2.310 | −7.765 | 1.036 | 1.00 | 80.47 | O |
| ATOM | 2441 | CB | LEU | B | 64 | −2.673 | −9.613 | −1.379 | 1.00 | 90.70 | C |
| ATOM | 2442 | CG | LEU | B | 64 | −2.000 | −10.949 | −1.716 | 1.00 | 94.68 | C |
| ATOM | 2443 | CD1 | LEU | B | 64 | −1.151 | −10.828 | −2.982 | 1.00 | 85.00 | C |
| ATOM | 2444 | CD2 | LEU | B | 64 | −1.148 | −11.432 | −0.555 | 1.00 | 75.29 | C |
| ATOM | 2445 | N | GLN | B | 65 | −4.423 | −7.307 | 0.465 | 1.00 | 87.14 | N |
| ATOM | 2446 | CA | GLN | B | 65 | −4.360 | −5.985 | 1.086 | 1.00 | 84.39 | C |
| ATOM | 2447 | C | GLN | B | 65 | −4.630 | −6.023 | 2.586 | 1.00 | 87.05 | C |
| ATOM | 2448 | O | GLN | B | 65 | −5.761 | −5.843 | 3.038 | 1.00 | 88.26 | O |
| ATOM | 2449 | CB | GLN | B | 65 | −5.282 | −4.984 | 0.391 | 1.00 | 93.23 | C |
| ATOM | 2450 | CG | GLN | B | 65 | −4.540 | −3.980 | −0.469 | 1.00 | 83.58 | C |
| ATOM | 2451 | CD | GLN | B | 65 | −5.472 | −3.055 | −1.222 | 1.00 | 97.96 | C |
| ATOM | 2452 | OE1 | GLN | B | 65 | −6.677 | −3.301 | −1.307 | 1.00 | 109.30 | O |
| ATOM | 2453 | NE2 | GLN | B | 65 | −4.917 | −1.984 | −1.780 | 1.00 | 81.55 | N |
| ATOM | 2454 | N | THR | B | 66 | −3.568 | −6.281 | 3.341 | 1.00 | 76.47 | N |
| ATOM | 2455 | CA | THR | B | 66 | −3.577 | −6.171 | 4.788 | 1.00 | 44.72 | C |
| ATOM | 2456 | C | THR | B | 66 | −2.803 | −4.909 | 5.109 | 1.00 | 50.95 | C |
| ATOM | 2457 | O | THR | B | 66 | −2.424 | −4.173 | 4.200 | 1.00 | 71.88 | O |
| ATOM | 2458 | CB | THR | B | 66 | −2.839 | −7.341 | 5.418 | 1.00 | 53.39 | C |
| ATOM | 2459 | OG1 | THR | B | 66 | −1.448 | −7.235 | 5.103 | 1.00 | 56.75 | O |
| ATOM | 2460 | CG2 | THR | B | 66 | −3.372 | −8.659 | 4.881 | 1.00 | 46.46 | C |
| ATOM | 2461 | N | LEU | B | 67 | −2.565 | −4.646 | 6.388 | 1.00 | 52.71 | N |
| ATOM | 2462 | CA | LEU | B | 67 | −1.746 | −3.502 | 6.780 | 1.00 | 49.66 | C |
| ATOM | 2463 | C | LEU | B | 67 | −0.286 | −3.833 | 6.540 | 1.00 | 53.48 | C |
| ATOM | 2464 | O | LEU | B | 67 | 0.467 | −3.034 | 5.983 | 1.00 | 52.47 | O |
| ATOM | 2465 | CB | LEU | B | 67 | −1.936 | −3.178 | 8.261 | 1.00 | 56.05 | C |
| ATOM | 2466 | CG | LEU | B | 67 | −3.098 | −2.281 | 8.664 | 1.00 | 45.36 | C |
| ATOM | 2467 | CD1 | LEU | B | 67 | −2.863 | −1.801 | 10.086 | 1.00 | 56.88 | C |
| ATOM | 2468 | CD2 | LEU | B | 67 | −3.214 | −1.109 | 7.710 | 1.00 | 42.61 | C |
| ATOM | 2469 | N | THR | B | 68 | 0.101 | −5.024 | 6.985 | 1.00 | 48.72 | N |
| ATOM | 2470 | CA | THR | B | 68 | 1.458 | −5.517 | 6.823 | 1.00 | 42.69 | C |
| ATOM | 2471 | C | THR | B | 68 | 1.927 | −5.319 | 5.403 | 1.00 | 41.03 | C |
| ATOM | 2472 | O | THR | B | 68 | 3.062 | −4.912 | 5.172 | 1.00 | 48.54 | O |
| ATOM | 2473 | CB | THR | B | 68 | 1.554 | −7.013 | 7.170 | 1.00 | 51.90 | C |
| ATOM | 2474 | OG1 | THR | B | 68 | 1.404 | −7.191 | 8.585 | 1.00 | 60.76 | O |
| ATOM | 2475 | CG2 | THR | B | 68 | 2.892 | −7.589 | 6.725 | 1.00 | 42.57 | C |
| ATOM | 2476 | N | ASN | B | 69 | 1.045 | −5.603 | 4.455 | 1.00 | 39.11 | N |
| ATOM | 2477 | CA | ASN | B | 69 | 1.398 | −5.536 | 3.045 | 1.00 | 44.58 | C |
| ATOM | 2478 | C | ASN | B | 69 | 1.478 | −4.109 | 2.528 | 1.00 | 43.73 | C |
| ATOM | 2479 | O | ASN | B | 69 | 2.077 | −3.853 | 1.482 | 1.00 | 45.50 | O |
| ATOM | 2480 | CB | ASN | B | 69 | 0.427 | −6.367 | 2.210 | 1.00 | 56.62 | C |
| ATOM | 2481 | CG | ASN | B | 69 | 0.622 | −7.849 | 2.414 | 1.00 | 49.98 | C |
| ATOM | 2482 | OD1 | ASN | B | 69 | 1.619 | −8.277 | 2.989 | 1.00 | 54.00 | O |
| ATOM | 2483 | ND2 | ASN | B | 69 | −0.324 | −8.640 | 1.946 | 1.00 | 61.34 | N |
| ATOM | 2484 | N | LEU | B | 70 | 0.873 | −3.187 | 3.265 | 1.00 | 37.97 | N |
| ATOM | 2485 | CA | LEU | B | 70 | 1.040 | −1.769 | 2.985 | 1.00 | 43.30 | C |
| ATOM | 2486 | C | LEU | B | 70 | 2.480 | −1.334 | 3.243 | 1.00 | 38.70 | C |
| ATOM | 2487 | O | LEU | B | 70 | 3.064 | −0.581 | 2.455 | 1.00 | 29.24 | O |
| ATOM | 2488 | CB | LEU | B | 70 | 0.081 | −0.939 | 3.834 | 1.00 | 53.28 | C |
| ATOM | 2489 | CG | LEU | B | 70 | −1.347 | −0.967 | 3.308 | 1.00 | 48.55 | C |
| ATOM | 2490 | CD1 | LEU | B | 70 | −2.190 | 0.083 | 4.010 | 1.00 | 45.61 | C |
| ATOM | 2491 | CD2 | LEU | B | 70 | −1.311 | −0.743 | 1.808 | 1.00 | 32.37 | C |
| ATOM | 2492 | N | PHE | B | 71 | 3.049 | −1.818 | 4.343 | 1.00 | 37.13 | N |
| ATOM | 2493 | CA | PHE | B | 71 | 4.453 | −1.547 | 4.655 | 1.00 | 45.05 | C |
| ATOM | 2494 | C | PHE | B | 71 | 5.405 | −2.276 | 3.705 | 1.00 | 37.96 | C |
| ATOM | 2495 | O | PHE | B | 71 | 6.486 | −1.771 | 3.374 | 1.00 | 31.22 | O |
| ATOM | 2496 | CB | PHE | B | 71 | 4.771 | −1.872 | 6.120 | 1.00 | 40.28 | C |
| ATOM | 2497 | CG | PHE | B | 71 | 3.952 | −1.080 | 7.095 | 1.00 | 37.11 | C |
| ATOM | 2498 | CD1 | PHE | B | 71 | 3.763 | 0.278 | 6.909 | 1.00 | 38.42 | C |
| ATOM | 2499 | CD2 | PHE | B | 71 | 3.365 | −1.692 | 8.192 | 1.00 | 42.01 | C |
| ATOM | 2500 | CE1 | PHE | B | 71 | 2.991 | 1.016 | 7.796 | 1.00 | 44.05 | C |
| ATOM | 2501 | CE2 | PHE | B | 71 | 2.597 | −0.961 | 9.087 | 1.00 | 35.22 | C |
| ATOM | 2502 | CZ | PHE | B | 71 | 2.409 | 0.396 | 8.890 | 1.00 | 35.08 | C |
| ATOM | 2503 | N | ILE | B | 72 | 4.979 | −3.455 | 3.257 | 1.00 | 41.92 | N |
| ATOM | 2504 | CA | ILE | B | 72 | 5.702 | −4.229 | 2.247 | 1.00 | 40.01 | C |
| ATOM | 2505 | C | ILE | B | 72 | 5.761 | −3.498 | 0.902 | 1.00 | 31.05 | C |
| ATOM | 2506 | O | ILE | B | 72 | 6.753 | −3.558 | 0.192 | 1.00 | 30.14 | O |
| ATOM | 2507 | CB | ILE | B | 72 | 5.033 | −5.593 | 2.039 | 1.00 | 41.02 | C |
| ATOM | 2508 | CG1 | ILE | B | 72 | 5.252 | −6.479 | 3.264 | 1.00 | 42.22 | C |
| ATOM | 2509 | CG2 | ILE | B | 72 | 5.570 | −6.273 | 0.802 | 1.00 | 41.85 | C |
| ATOM | 2510 | CD1 | ILE | B | 72 | 6.570 | −7.163 | 3.282 | 1.00 | 40.80 | C |
| ATOM | 2511 | N | THR | B | 73 | 4.685 | −2.815 | 0.551 | 1.00 | 31.86 | N |
| ATOM | 2512 | CA | THR | B | 73 | 4.678 | −2.022 | −0.662 | 1.00 | 34.12 | C |
| ATOM | 2513 | C | THR | B | 73 | 5.704 | −0.905 | −0.561 | 1.00 | 32.79 | C |
| ATOM | 2514 | O | THR | B | 73 | 6.408 | −0.600 | −1.519 | 1.00 | 30.72 | O |
| ATOM | 2515 | CB | THR | B | 73 | 3.293 | −1.415 | −0.939 | 1.00 | 37.75 | C |
| ATOM | 2516 | OG1 | THR | B | 73 | 2.352 | −2.465 | −1.189 | 1.00 | 35.86 | O |
| ATOM | 2517 | CG2 | THR | B | 73 | 3.350 | −0.502 | −2.153 | 1.00 | 36.94 | C |
| ATOM | 2518 | N | SER | B | 74 | 5.778 | −0.296 | 0.613 | 1.00 | 37.11 | N |
| ATOM | 2519 | CA | SER | B | 74 | 6.711 | 0.793 | 0.859 | 1.00 | 34.77 | C |
| ATOM | 2520 | C | SER | B | 74 | 8.114 | 0.276 | 0.613 | 1.00 | 34.92 | C |
| ATOM | 2521 | O | SER | B | 74 | 8.948 | 0.936 | −0.008 | 1.00 | 33.69 | O |
| ATOM | 2522 | CB | SER | B | 74 | 6.566 | 1.291 | 2.297 | 1.00 | 32.39 | C |
| ATOM | 2523 | OG | SER | B | 74 | 7.507 | 2.301 | 2.586 | 1.00 | 40.13 | O |
| ATOM | 2524 | N | LEU | B | 75 | 8.353 | −0.930 | 1.106 | 1.00 | 36.86 | N |
| ATOM | 2525 | CA | LEU | B | 75 | 9.599 | −1.638 | 0.886 | 1.00 | 28.84 | C |
| ATOM | 2526 | C | LEU | B | 75 | 9.837 | −1.875 | −0.607 | 1.00 | 33.13 | C |
| ATOM | 2527 | O | LEU | B | 75 | 10.937 | −1.637 | −1.106 | 1.00 | 39.30 | O |
| ATOM | 2528 | CB | LEU | B | 75 | 9.538 | −2.969 | 1.620 | 1.00 | 32.88 | C |
| ATOM | 2529 | CG | LEU | B | 75 | 10.786 | −3.413 | 2.354 | 1.00 | 33.06 | C |
| ATOM | 2530 | CD1 | LEU | B | 75 | 11.353 | −2.244 | 3.120 | 1.00 | 33.18 | C |
| ATOM | 2531 | CD2 | LEU | B | 75 | 10.438 | −4.572 | 3.273 | 1.00 | 36.25 | C |
| ATOM | 2532 | N | ALA | B | 76 | 8.808 | −2.345 | −1.313 | 1.00 | 30.13 | N |
| ATOM | 2533 | CA | ALA | B | 76 | 8.895 | −2.580 | −2.758 | 1.00 | 33.92 | C |
| ATOM | 2534 | C | ALA | B | 76 | 9.292 | −1.321 | −3.531 | 1.00 | 36.72 | C |
| ATOM | 2535 | O | ALA | B | 76 | 10.150 | −1.369 | −4.410 | 1.00 | 40.26 | O |
| ATOM | 2536 | CB | ALA | B | 76 | 7.585 | −3.142 | −3.298 | 1.00 | 31.56 | C |
| ATOM | 2537 | N | CYS | B | 77 | 8.669 | −0.196 | −3.200 | 1.00 | 33.85 | N |
| ATOM | 2538 | CA | CYS | B | 77 | 8.981 | 1.061 | −3.862 | 1.00 | 28.40 | C |
| ATOM | 2539 | C | CYS | B | 77 | 10.425 | 1.487 | −3.663 | 1.00 | 27.87 | C |
| ATOM | 2540 | O | CYS | B | 77 | 11.037 | 2.021 | −4.579 | 1.00 | 27.46 | O |
| ATOM | 2541 | CB | CYS | B | 77 | 8.048 | 2.170 | −3.384 | 1.00 | 21.53 | C |
| ATOM | 2542 | SG | CYS | B | 77 | 6.368 | 2.029 | −4.017 | 1.00 | 51.06 | S |
| ATOM | 2543 | N | ALA | B | 78 | 10.971 | 1.271 | −2.472 | 1.00 | 26.71 | N |
| ATOM | 2544 | CA | ALA | B | 78 | 12.368 | 1.625 | −2.250 | 1.00 | 32.36 | C |
| ATOM | 2545 | C | ALA | B | 78 | 13.305 | 0.716 | −3.048 | 1.00 | 34.78 | C |
| ATOM | 2546 | O | ALA | B | 78 | 14.376 | 1.143 | −3.454 | 1.00 | 38.39 | O |
| ATOM | 2547 | CB | ALA | B | 78 | 12.717 | 1.603 | −0.778 | 1.00 | 26.78 | C |
| ATOM | 2548 | N | ASP | B | 79 | 12.896 | −0.529 | −3.282 | 1.00 | 33.95 | N |
| ATOM | 2549 | CA | ASP | B | 79 | 13.678 | −1.425 | −4.129 | 1.00 | 33.38 | C |
| ATOM | 2550 | C | ASP | B | 79 | 13.545 | −1.037 | −5.610 | 1.00 | 35.56 | C |
| ATOM | 2551 | O | ASP | B | 79 | 14.478 | −1.208 | −6.397 | 1.00 | 34.90 | O |
| ATOM | 2552 | CB | ASP | B | 79 | 13.294 | −2.884 | −3.882 | 1.00 | 30.44 | C |
| ATOM | 2553 | CG | ASP | B | 79 | 13.618 | −3.342 | −2.454 | 1.00 | 60.19 | C |
| ATOM | 2554 | OD1 | ASP | B | 79 | 14.679 | −2.930 | −1.915 | 1.00 | 51.01 | O |
| ATOM | 2555 | OD2 | ASP | B | 79 | 12.812 | −4.116 | −1.873 | 1.00 | 63.48 | O |
| ATOM | 2556 | N | LEU | B | 80 | 12.395 | −0.479 | −5.971 | 1.00 | 26.75 | N |
| ATOM | 2557 | CA | LEU | B | 80 | 12.155 | −0.022 | −7.330 | 1.00 | 28.95 | C |
| ATOM | 2558 | C | LEU | B | 80 | 13.040 | 1.165 | −7.705 | 1.00 | 34.79 | C |
| ATOM | 2559 | O | LEU | B | 80 | 13.729 | 1.146 | −8.726 | 1.00 | 40.98 | O |
| ATOM | 2560 | CB | LEU | B | 80 | 10.687 | 0.332 | −7.521 | 1.00 | 37.91 | C |
| ATOM | 2561 | CG | LEU | B | 80 | 10.135 | 0.051 | −8.917 | 1.00 | 40.15 | C |
| ATOM | 2562 | CD1 | LEU | B | 80 | 10.313 | −1.419 | −9.283 | 1.00 | 38.78 | C |
| ATOM | 2563 | CD2 | LEU | B | 80 | 8.678 | 0.451 | −8.995 | 1.00 | 28.80 | C |
| ATOM | 2564 | N | VAL | B | 81 | 13.018 | 2.205 | −6.886 | 1.00 | 39.16 | N |
| ATOM | 2565 | CA | VAL | B | 81 | 13.944 | 3.317 | −7.069 | 1.00 | 41.38 | C |
| ATOM | 2566 | C | VAL | B | 81 | 15.368 | 2.799 | −7.331 | 1.00 | 35.74 | C |
| ATOM | 2567 | O | VAL | B | 81 | 16.044 | 3.271 | −8.238 | 1.00 | 37.28 | O |
| ATOM | 2568 | CB | VAL | B | 81 | 13.932 | 4.290 | −5.855 | 1.00 | 40.58 | C |
| ATOM | 2569 | CG1 | VAL | B | 81 | 14.881 | 5.445 | −6.089 | 1.00 | 31.34 | C |
| ATOM | 2570 | CG2 | VAL | B | 81 | 12.518 | 4.824 | −5.600 | 1.00 | 32.21 | C |
| ATOM | 2571 | N | VAL | B | 82 | 15.816 | 1.817 | −6.553 | 1.00 | 27.77 | N |
| ATOM | 2572 | CA | VAL | B | 82 | 17.155 | 1.253 | −6.730 | 1.00 | 34.85 | C |
| ATOM | 2573 | C | VAL | B | 82 | 17.349 | 0.555 | −8.086 | 1.00 | 44.70 | C |
| ATOM | 2574 | O | VAL | B | 82 | 18.363 | 0.768 | −8.770 | 1.00 | 35.87 | O |
| ATOM | 2575 | CB | VAL | B | 82 | 17.523 | 0.270 | −5.598 | 1.00 | 28.58 | C |
| ATOM | 2576 | CG1 | VAL | B | 82 | 18.834 | −0.462 | −5.912 | 1.00 | 25.88 | C |
| ATOM | 2577 | CG2 | VAL | B | 82 | 17.626 | 1.003 | −4.292 | 1.00 | 25.57 | C |
| ATOM | 2578 | N | GLY | B | 83 | 16.385 | −0.282 | −8.468 | 1.00 | 37.50 | N |
| ATOM | 2579 | CA | GLY | B | 83 | 16.448 | −0.966 | −9.747 | 1.00 | 41.11 | C |
| ATOM | 2580 | C | GLY | B | 83 | 16.366 | −0.015 | −10.933 | 1.00 | 35.83 | C |
| ATOM | 2581 | O | GLY | B | 83 | 16.933 | −0.276 | −11.993 | 1.00 | 25.50 | O |
| ATOM | 2582 | N | LEU | B | 84 | 15.658 | 1.094 | −10.742 | 1.00 | 33.48 | N |
| ATOM | 2583 | CA | LEU | B | 84 | 15.450 | 2.074 | −11.800 | 1.00 | 34.52 | C |
| ATOM | 2584 | C | LEU | B | 84 | 16.506 | 3.183 | −11.835 | 1.00 | 38.42 | C |
| ATOM | 2585 | O | LEU | B | 84 | 16.931 | 3.593 | −12.915 | 1.00 | 41.82 | O |
| ATOM | 2586 | CB | LEU | B | 84 | 14.062 | 2.708 | −11.680 | 1.00 | 38.40 | C |
| ATOM | 2587 | CG | LEU | B | 84 | 12.832 | 1.868 | −12.011 | 1.00 | 34.14 | C |
| ATOM | 2588 | CD1 | LEU | B | 84 | 11.606 | 2.750 | −11.945 | 1.00 | 21.05 | C |
| ATOM | 2589 | CD2 | LEU | B | 84 | 12.976 | 1.226 | −13.379 | 1.00 | 29.95 | C |
| ATOM | 2590 | N | LEU | B | 85 | 16.918 | 3.680 | −10.672 | 1.00 | 32.76 | N |
| ATOM | 2591 | CA | LEU | B | 85 | 17.874 | 4.788 | −10.632 | 1.00 | 30.47 | C |
| ATOM | 2592 | C | LEU | B | 85 | 19.248 | 4.429 | −10.063 | 1.00 | 33.38 | C |
| ATOM | 2593 | O | LEU | B | 85 | 20.268 | 4.643 | −10.715 | 1.00 | 36.10 | O |
| ATOM | 2594 | CB | LEU | B | 85 | 17.278 | 5.985 | −9.887 | 1.00 | 34.29 | C |
| ATOM | 2595 | CG | LEU | B | 85 | 16.057 | 6.551 | −10.611 | 1.00 | 34.40 | C |
| ATOM | 2596 | CD1 | LEU | B | 85 | 15.509 | 7.793 | −9.949 | 1.00 | 31.74 | C |
| ATOM | 2597 | CD2 | LEU | B | 85 | 16.442 | 6.852 | −12.040 | 1.00 | 42.40 | C |
| ATOM | 2598 | N | VAL | B | 86 | 19.281 | 3.875 | −8.857 | 1.00 | 30.60 | N |
| ATOM | 2599 | CA | VAL | B | 86 | 20.562 | 3.618 | −8.211 | 1.00 | 33.48 | C |
| ATOM | 2600 | C | VAL | B | 86 | 21.481 | 2.679 | −9.010 | 1.00 | 30.28 | C |
| ATOM | 2601 | O | VAL | B | 86 | 22.579 | 3.070 | −9.368 | 1.00 | 31.55 | O |
| ATOM | 2602 | CB | VAL | B | 86 | 20.388 | 3.115 | −6.770 | 1.00 | 26.48 | C |
| ATOM | 2603 | CG1 | VAL | B | 86 | 21.734 | 3.074 | −6.059 | 1.00 | 27.28 | C |
| ATOM | 2604 | CG2 | VAL | B | 86 | 19.438 | 4.012 | −6.037 | 1.00 | 26.79 | C |
| ATOM | 2605 | N | VAL | B | 87 | 21.032 | 1.459 | −9.296 | 1.00 | 34.56 | N |
| ATOM | 2606 | CA | VAL | B | 87 | 21.886 | 0.462 | −9.954 | 1.00 | 35.58 | C |
| ATOM | 2607 | C | VAL | B | 87 | 22.273 | 0.837 | −11.388 | 1.00 | 39.16 | C |
| ATOM | 2608 | O | VAL | B | 87 | 23.428 | 0.675 | −11.772 | 1.00 | 42.29 | O |
| ATOM | 2609 | CB | VAL | B | 87 | 21.275 | −0.969 | −9.939 | 1.00 | 35.29 | C |
| ATOM | 2610 | CG1 | VAL | B | 87 | 21.891 | −1.822 | −11.046 | 1.00 | 25.30 | C |
| ATOM | 2611 | CG2 | VAL | B | 87 | 21.476 | −1.625 | −8.578 | 1.00 | 29.44 | C |
| ATOM | 2612 | N | PRO | B | 88 | 21.307 | 1.320 | −12.190 | 1.00 | 35.02 | N |
| ATOM | 2613 | CA | PRO | B | 88 | 21.637 | 1.830 | −13.528 | 1.00 | 33.34 | C |
| ATOM | 2614 | C | PRO | B | 88 | 22.849 | 2.783 | −13.564 | 1.00 | 37.40 | C |
| ATOM | 2615 | O | PRO | B | 88 | 23.840 | 2.457 | −14.213 | 1.00 | 34.67 | O |
| ATOM | 2616 | CB | PRO | B | 88 | 20.350 | 2.539 | −13.951 | 1.00 | 26.86 | C |
| ATOM | 2617 | CG | PRO | B | 88 | 19.275 | 1.711 | −13.305 | 1.00 | 29.33 | C |
| ATOM | 2618 | CD | PRO | B | 88 | 19.849 | 1.197 | −11.995 | 1.00 | 33.93 | C |
| ATOM | 2619 | N | PHE | B | 89 | 22.785 | 3.929 | −12.891 | 1.00 | 33.65 | N |
| ATOM | 2620 | CA | PHE | B | 89 | 23.927 | 4.841 | −12.883 | 1.00 | 31.24 | C |
| ATOM | 2621 | C | PHE | B | 89 | 25.144 | 4.184 | −12.218 | 1.00 | 36.97 | C |
| ATOM | 2622 | O | PHE | B | 89 | 26.290 | 4.434 | −12.602 | 1.00 | 33.35 | O |
| ATOM | 2623 | CB | PHE | B | 89 | 23.613 | 6.098 | −12.091 | 1.00 | 32.08 | C |
| ATOM | 2624 | CG | PHE | B | 89 | 22.688 | 7.058 | −12.766 | 1.00 | 26.57 | C |
| ATOM | 2625 | CD1 | PHE | B | 89 | 23.178 | 8.239 | −13.316 | 1.00 | 30.15 | C |
| ATOM | 2626 | CD2 | PHE | B | 89 | 21.321 | 6.826 | −12.782 | 1.00 | 30.09 | C |
| ATOM | 2627 | CE1 | PHE | B | 89 | 22.321 | 9.171 | −13.896 | 1.00 | 31.45 | C |
| ATOM | 2628 | CE2 | PHE | B | 89 | 20.450 | 7.737 | −13.366 | 1.00 | 26.89 | C |
| ATOM | 2629 | CZ | PHE | B | 89 | 20.950 | 8.916 | −13.923 | 1.00 | 35.92 | C |
| ATOM | 2630 | N | GLY | B | 90 | 24.895 | 3.375 | −11.192 | 1.00 | 31.65 | N |
| ATOM | 2631 | CA | GLY | B | 90 | 25.963 | 2.717 | −10.462 | 1.00 | 27.50 | C |
| ATOM | 2632 | C | GLY | B | 90 | 26.708 | 1.711 | −11.317 | 1.00 | 35.94 | C |
| ATOM | 2633 | O | GLY | B | 90 | 27.901 | 1.492 | −11.149 | 1.00 | 27.01 | O |
| ATOM | 2634 | N | ALA | B | 91 | 25.987 | 1.095 | −12.245 | 1.00 | 43.48 | N |
| ATOM | 2635 | CA | ALA | B | 91 | 26.577 | 0.185 | −13.213 | 1.00 | 41.13 | C |
| ATOM | 2636 | C | ALA | B | 91 | 27.630 | 0.883 | −14.098 | 1.00 | 38.74 | C |
| ATOM | 2637 | O | ALA | B | 91 | 28.755 | 0.398 | −14.236 | 1.00 | 34.38 | O |
| ATOM | 2638 | CB | ALA | B | 91 | 25.486 | −0.434 | −14.062 | 1.00 | 33.13 | C |
| ATOM | 2639 | N | THR | B | 92 | 27.259 | 2.015 | −14.695 | 1.00 | 28.35 | N |
| ATOM | 2640 | CA | THR | B | 92 | 28.180 | 2.785 | −15.524 | 1.00 | 26.92 | C |
| ATOM | 2641 | C | THR | B | 92 | 29.466 | 3.085 | −14.759 | 1.00 | 34.07 | C |
| ATOM | 2642 | O | THR | B | 92 | 30.560 | 2.964 | −15.297 | 1.00 | 34.06 | O |
| ATOM | 2643 | CB | THR | B | 92 | 27.554 | 4.122 | −16.052 | 1.00 | 34.13 | C |
| ATOM | 2644 | OG1 | THR | B | 92 | 27.572 | 5.137 | −15.033 | 1.00 | 33.42 | O |
| ATOM | 2645 | CG2 | THR | B | 92 | 26.137 | 3.903 | −16.527 | 1.00 | 29.55 | C |
| ATOM | 2646 | N | LEU | B | 93 | 29.327 | 3.466 | −13.496 | 1.00 | 34.88 | N |
| ATOM | 2647 | CA | LEU | B | 93 | 30.483 | 3.767 | −12.666 | 1.00 | 35.78 | C |
| ATOM | 2648 | C | LEU | B | 93 | 31.418 | 2.565 | −12.502 | 1.00 | 31.13 | C |
| ATOM | 2649 | O | LEU | B | 93 | 32.633 | 2.719 | −12.431 | 1.00 | 34.51 | O |
| ATOM | 2650 | CB | LEU | B | 93 | 30.024 | 4.274 | −11.298 | 1.00 | 35.00 | C |
| ATOM | 2651 | CG | LEU | B | 93 | 31.058 | 4.775 | −10.289 | 1.00 | 28.87 | C |
| ATOM | 2652 | CD1 | LEU | B | 93 | 32.032 | 5.766 | −10.917 | 1.00 | 19.28 | C |
| ATOM | 2653 | CD2 | LEU | B | 93 | 30.308 | 5.426 | −9.147 | 1.00 | 31.84 | C |
| ATOM | 2654 | N | VAL | B | 94 | 30.856 | 1.369 | −12.427 | 1.00 | 33.68 | N |
| ATOM | 2655 | CA | VAL | B | 94 | 31.674 | 0.194 | −12.168 | 1.00 | 42.30 | C |
| ATOM | 2656 | C | VAL | B | 94 | 32.275 | −0.351 | −13.463 | 1.00 | 53.81 | C |
| ATOM | 2657 | O | VAL | B | 94 | 33.399 | −0.865 | −13.473 | 1.00 | 47.71 | O |
| ATOM | 2658 | CB | VAL | B | 94 | 30.889 | −0.917 | −11.446 | 1.00 | 44.56 | C |
| ATOM | 2659 | CG1 | VAL | B | 94 | 31.857 | −1.913 | −10.841 | 1.00 | 38.11 | C |
| ATOM | 2660 | CG2 | VAL | B | 94 | 30.010 | −0.321 | −10.359 | 1.00 | 50.06 | C |
| ATOM | 2661 | N | VAL | B | 95 | 31.527 | −0.226 | −14.555 | 1.00 | 40.59 | N |
| ATOM | 2662 | CA | VAL | B | 95 | 32.018 | −0.648 | −15.858 | 1.00 | 43.57 | C |
| ATOM | 2663 | C | VAL | B | 95 | 33.120 | 0.275 | −16.405 | 1.00 | 46.90 | C |
| ATOM | 2664 | O | VAL | B | 95 | 34.093 | −0.194 | −16.986 | 1.00 | 52.48 | O |
| ATOM | 2665 | CB | VAL | B | 95 | 30.870 | −0.768 | −16.879 | 1.00 | 38.40 | C |
| ATOM | 2666 | CG1 | VAL | B | 95 | 31.416 | −1.095 | −18.246 | 1.00 | 56.62 | C |
| ATOM | 2667 | CG2 | VAL | B | 95 | 29.877 | −1.836 | −16.436 | 1.00 | 53.81 | C |
| ATOM | 2668 | N | ARG | B | 96 | 32.978 | 1.581 | −16.203 | 1.00 | 41.13 | N |
| ATOM | 2669 | CA | ARG | B | 96 | 33.899 | 2.543 | −16.796 | 1.00 | 31.67 | C |
| ATOM | 2670 | C | ARG | B | 96 | 34.944 | 3.116 | −15.851 | 1.00 | 36.55 | C |
| ATOM | 2671 | O | ARG | B | 96 | 35.901 | 3.755 | −16.297 | 1.00 | 45.67 | O |
| ATOM | 2672 | CB | ARG | B | 96 | 33.135 | 3.683 | −17.455 | 1.00 | 30.76 | C |
| ATOM | 2673 | CG | ARG | B | 96 | 32.608 | 3.335 | −18.853 | 1.00 | 54.47 | C |
| ATOM | 2674 | CD | ARG | B | 96 | 33.752 | 3.100 | −19.845 | 1.00 | 44.11 | C |
| ATOM | 2675 | NE | ARG | B | 96 | 34.588 | 4.286 | −20.026 | 1.00 | 37.39 | N |
| ATOM | 2676 | CZ | ARG | B | 96 | 34.200 | 5.367 | −20.703 | 1.00 | 42.82 | C |
| ATOM | 2677 | NH1 | ARG | B | 96 | 32.990 | 5.408 | −21.253 | 1.00 | 26.41 | N |
| ATOM | 2678 | NH2 | ARG | B | 96 | 35.019 | 6.407 | −20.824 | 1.00 | 32.70 | N |
| ATOM | 2679 | N | GLY | B | 97 | 34.767 | 2.890 | −14.556 | 1.00 | 33.05 | N |
| ATOM | 2680 | CA | GLY | B | 97 | 35.631 | 3.484 | −13.557 | 1.00 | 25.63 | C |
| ATOM | 2681 | C | GLY | B | 97 | 35.668 | 5.007 | −13.587 | 1.00 | 34.01 | C |
| ATOM | 2682 | O | GLY | B | 97 | 36.683 | 5.599 | −13.229 | 1.00 | 44.61 | O |
| ATOM | 2683 | N | THR | B | 98 | 34.578 | 5.644 | −14.021 | 1.00 | 25.76 | N |
| ATOM | 2684 | CA | THR | B | 98 | 34.487 | 7.104 | −14.044 | 1.00 | 27.04 | C |
| ATOM | 2685 | C | THR | B | 98 | 33.032 | 7.473 | −13.984 | 1.00 | 30.83 | C |
| ATOM | 2686 | O | THR | B | 98 | 32.168 | 6.626 | −14.199 | 1.00 | 34.86 | O |
| ATOM | 2687 | CB | THR | B | 98 | 35.035 | 7.739 | −15.347 | 1.00 | 31.16 | C |
| ATOM | 2688 | OG1 | THR | B | 98 | 35.961 | 6.853 | −15.971 | 1.00 | 39.15 | O |
| ATOM | 2689 | CG2 | THR | B | 98 | 35.705 | 9.078 | −15.068 | 1.00 | 21.21 | C |
| ATOM | 2690 | N | TRP | B | 99 | 32.767 | 8.748 | −13.719 | 1.00 | 23.92 | N |
| ATOM | 2691 | CA | TRP | B | 99 | 31.411 | 9.257 | −13.680 | 1.00 | 21.86 | C |
| ATOM | 2692 | C | TRP | B | 99 | 31.143 | 10.058 | −14.940 | 1.00 | 25.43 | C |
| ATOM | 2693 | O | TRP | B | 99 | 31.815 | 11.055 | −15.191 | 1.00 | 27.16 | O |
| ATOM | 2694 | CB | TRP | B | 99 | 31.183 | 10.102 | −12.426 | 1.00 | 19.01 | O |
| ATOM | 2695 | CG | TRP | B | 99 | 29.743 | 10.435 | −12.193 | 1.00 | 21.61 | C |
| ATOM | 2696 | CD1 | TRP | B | 99 | 29.163 | 11.656 | −12.324 | 1.00 | 21.40 | C |
| ATOM | 2697 | CD2 | TRP | B | 99 | 28.697 | 9.532 | −11.801 | 1.00 | 16.86 | C |
| ATOM | 2698 | NE1 | TRP | B | 99 | 27.826 | 11.579 | −12.024 | 1.00 | 27.38 | N |
| ATOM | 2699 | CE2 | TRP | B | 99 | 27.514 | 10.288 | −11.696 | 1.00 | 23.16 | C |
| ATOM | 2700 | CE3 | TRP | B | 99 | 28.649 | 8.163 | −11.518 | 1.00 | 19.92 | C |
| ATOM | 2701 | CZ2 | TRP | B | 99 | 26.290 | 9.720 | −11.336 | 1.00 | 19.09 | C |
| ATOM | 2702 | CZ3 | TRP | B | 99 | 27.436 | 7.597 | −11.147 | 1.00 | 18.27 | C |
| ATOM | 2703 | CH2 | TRP | B | 99 | 26.274 | 8.373 | −11.067 | 1.00 | 21.87 | C |
| ATOM | 2704 | N | LEU | B | 100 | 30.162 | 9.617 | −15.731 | 1.00 | 24.59 | N |
| ATOM | 2705 | CA | LEU | B | 100 | 29.878 | 10.249 | −17.022 | 1.00 | 25.59 | C |
| ATOM | 2706 | C | LEU | B | 100 | 28.863 | 11.378 | −16.925 | 1.00 | 28.75 | C |
| ATOM | 2707 | O | LEU | B | 100 | 28.643 | 12.101 | −17.897 | 1.00 | 27.21 | O |
| ATOM | 2708 | CB | LEU | B | 100 | 29.326 | 9.239 | −18.032 | 1.00 | 28.56 | C |
| ATOM | 2709 | CG | LEU | B | 100 | 29.990 | 7.945 | −18.520 | 1.00 | 34.85 | C |
| ATOM | 2710 | CD1 | LEU | B | 100 | 29.339 | 7.582 | −19.841 | 1.00 | 33.91 | C |
| ATOM | 2711 | CD2 | LEU | B | 100 | 31.503 | 8.054 | −18.694 | 1.00 | 32.67 | C |
| ATOM | 2712 | N | TRP | B | 101 | 28.242 | 11.538 | −15.764 | 1.00 | 26.88 | N |
| ATOM | 2713 | CA | TRP | B | 101 | 26.988 | 12.275 | −15.714 | 1.00 | 24.26 | C |
| ATOM | 2714 | C | TRP | B | 101 | 27.027 | 13.662 | −15.063 | 1.00 | 24.51 | C |
| ATOM | 2715 | O | TRP | B | 101 | 26.021 | 14.352 | −15.030 | 1.00 | 28.72 | O |
| ATOM | 2716 | CB | TRP | B | 101 | 25.908 | 11.380 | −15.103 | 1.00 | 22.53 | C |
| ATOM | 2717 | CG | TRP | B | 101 | 25.946 | 9.988 | −15.686 | 1.00 | 25.58 | C |
| ATOM | 2718 | CD1 | TRP | B | 101 | 26.400 | 8.852 | −15.078 | 1.00 | 23.85 | C |
| ATOM | 2719 | CD2 | TRP | B | 101 | 25.548 | 9.601 | −17.005 | 1.00 | 24.91 | C |
| ATOM | 2720 | NE1 | TRP | B | 101 | 26.299 | 7.776 | −15.933 | 1.00 | 23.03 | N |
| ATOM | 2721 | CE2 | TRP | B | 101 | 25.780 | 8.207 | −17.122 | 1.00 | 23.56 | C |
| ATOM | 2722 | CE3 | TRP | B | 101 | 25.006 | 10.289 | −18.094 | 1.00 | 21.69 | C |
| ATOM | 2723 | CZ2 | TRP | B | 101 | 25.480 | 7.491 | −18.278 | 1.00 | 22.78 | C |
| ATOM | 2724 | CZ3 | TRP | B | 101 | 24.714 | 9.577 | −19.254 | 1.00 | 22.46 | C |
| ATOM | 2725 | CH2 | TRP | B | 101 | 24.955 | 8.191 | −19.335 | 1.00 | 26.38 | C |
| ATOM | 2726 | N | GLY | B | 102 | 28.185 | 14.082 | −14.566 | 1.00 | 30.91 | N |
| ATOM | 2727 | CA | GLY | B | 102 | 28.325 | 15.438 | −14.057 | 1.00 | 23.54 | C |
| ATOM | 2728 | C | GLY | B | 102 | 28.298 | 15.578 | −12.542 | 1.00 | 28.47 | C |
| ATOM | 2729 | O | GLY | B | 102 | 27.739 | 14.745 | −11.824 | 1.00 | 28.62 | O |
| ATOM | 2730 | N | SER | B | 103 | 28.892 | 16.652 | −12.043 | 1.00 | 22.82 | N |
| ATOM | 2731 | CA | SER | B | 103 | 28.974 | 16.835 | −10.599 | 1.00 | 25.49 | C |
| ATOM | 2732 | C | SER | B | 103 | 27.596 | 16.851 | −9.932 | 1.00 | 29.19 | C |
| ATOM | 2733 | O | SER | B | 103 | 27.390 | 16.222 | −8.899 | 1.00 | 29.30 | O |
| ATOM | 2734 | CB | SER | B | 103 | 29.760 | 18.102 | −10.250 | 1.00 | 25.41 | C |
| ATOM | 2735 | OG | SER | B | 103 | 31.049 | 18.089 | −10.853 | 1.00 | 40.23 | O |
| ATOM | 2736 | N | PHE | B | 104 | 26.647 | 17.566 | −10.516 | 1.00 | 24.16 | N |
| ATOM | 2737 | CA | PHE | B | 104 | 25.350 | 17.671 | −9.876 | 1.00 | 24.80 | C |
| ATOM | 2738 | C | PHE | B | 104 | 24.639 | 16.332 | −9.772 | 1.00 | 27.15 | C |
| ATOM | 2739 | O | PHE | B | 104 | 24.100 | 16.003 | −8.718 | 1.00 | 31.52 | O |
| ATOM | 2740 | CB | PHE | B | 104 | 24.443 | 18.671 | −10.583 | 1.00 | 30.47 | C |
| ATOM | 2741 | CG | PHE | B | 104 | 23.043 | 18.665 | −10.061 | 1.00 | 26.80 | C |
| ATOM | 2742 | CD1 | PHE | B | 104 | 22.780 | 19.077 | −8.764 | 1.00 | 26.69 | C |
| ATOM | 2743 | CD2 | PHE | B | 104 | 21.994 | 18.221 | −10.850 | 1.00 | 31.36 | C |
| ATOM | 2744 | CE1 | PHE | B | 104 | 21.499 | 19.077 | −8.271 | 1.00 | 21.80 | C |
| ATOM | 2745 | CE2 | PHE | B | 104 | 20.702 | 18.211 | −10.358 | 1.00 | 34.74 | C |
| ATOM | 2746 | CZ | PHE | B | 104 | 20.456 | 18.641 | −9.064 | 1.00 | 31.83 | C |
| ATOM | 2747 | N | LEU | B | 105 | 24.621 | 15.568 | −10.863 | 1.00 | 30.32 | N |
| ATOM | 2748 | CA | LEU | B | 105 | 23.959 | 14.271 | −10.840 | 1.00 | 25.78 | C |
| ATOM | 2749 | C | LEU | B | 105 | 24.687 | 13.305 | −9.920 | 1.00 | 25.33 | C |
| ATOM | 2750 | O | LEU | B | 105 | 24.073 | 12.372 | −9.395 | 1.00 | 23.85 | O |
| ATOM | 2751 | CB | LEU | B | 105 | 23.819 | 13.671 | −12.238 | 1.00 | 23.80 | C |
| ATOM | 2752 | CG | LEU | B | 105 | 22.712 | 14.163 | −13.172 | 1.00 | 28.25 | C |
| ATOM | 2753 | CD1 | LEU | B | 105 | 22.323 | 13.042 | −14.142 | 1.00 | 21.96 | C |
| ATOM | 2754 | CD2 | LEU | B | 105 | 21.498 | 14.631 | −12.395 | 1.00 | 29.70 | C |
| ATOM | 2755 | N | CYS | B | 106 | 25.989 | 13.525 | −9.727 | 1.00 | 22.99 | N |
| ATOM | 2756 | CA | CYS | B | 106 | 26.765 | 12.688 | −8.814 | 1.00 | 23.54 | C |
| ATOM | 2757 | C | CYS | B | 106 | 26.190 | 12.797 | −7.406 | 1.00 | 28.97 | C |
| ATOM | 2758 | O | CYS | B | 106 | 25.909 | 11.779 | −6.757 | 1.00 | 25.84 | O |
| ATOM | 2759 | CB | CYS | B | 106 | 28.244 | 13.083 | −8.802 | 1.00 | 24.33 | C |
| ATOM | 2760 | SG | CYS | B | 106 | 29.251 | 12.142 | −7.612 | 1.00 | 25.01 | S |
| ATOM | 2761 | N | GLU | B | 107 | 26.013 | 14.038 | −6.947 | 1.00 | 26.78 | N |
| ATOM | 2762 | CA | GLU | B | 107 | 25.500 | 14.301 | −5.614 | 1.00 | 23.89 | C |
| ATOM | 2763 | C | GLU | B | 107 | 24.088 | 13.794 | −5.483 | 1.00 | 23.83 | C |
| ATOM | 2764 | O | GLU | B | 107 | 23.775 | 13.074 | −4.546 | 1.00 | 17.95 | O |
| ATOM | 2765 | CB | GLU | B | 107 | 25.571 | 15.785 | −5.279 | 1.00 | 31.31 | C |
| ATOM | 2766 | CG | GLU | B | 107 | 26.677 | 16.110 | −4.283 | 1.00 | 53.35 | C |
| ATOM | 2767 | CD | GLU | B | 107 | 26.605 | 17.535 | −3.760 | 1.00 | 71.43 | C |
| ATOM | 2768 | OE1 | GLU | B | 107 | 27.093 | 17.781 | −2.622 | 1.00 | 59.99 | O |
| ATOM | 2769 | OE2 | GLU | B | 107 | 26.057 | 18.396 | −4.493 | 1.00 | 67.52 | O |
| ATOM | 2770 | N | LEU | B | 108 | 23.250 | 14.155 | −6.450 | 1.00 | 26.78 | N |
| ATOM | 2771 | CA | LEU | B | 108 | 21.870 | 13.669 | −6.505 | 1.00 | 28.67 | C |
| ATOM | 2772 | C | LEU | B | 108 | 21.768 | 12.149 | −6.419 | 1.00 | 25.50 | C |
| ATOM | 2773 | O | LEU | B | 108 | 20.956 | 11.624 | −5.659 | 1.00 | 29.97 | O |
| ATOM | 2774 | CB | LEU | B | 108 | 21.166 | 14.153 | −7.771 | 1.00 | 23.10 | C |
| ATOM | 2775 | CG | LEU | B | 108 | 19.660 | 13.978 | −7.655 | 1.00 | 23.03 | C |
| ATOM | 2776 | CD1 | LEU | B | 108 | 19.157 | 14.893 | −6.542 | 1.00 | 23.50 | C |
| ATOM | 2777 | CD2 | LEU | B | 108 | 18.967 | 14.276 | −8.996 | 1.00 | 21.21 | C |
| ATOM | 2778 | N | TRP | B | 109 | 22.585 | 11.456 | −7.211 | 1.00 | 25.65 | N |
| ATOM | 2779 | CA | TRP | B | 109 | 22.618 | 9.999 | −7.223 | 1.00 | 22.11 | C |
| ATOM | 2780 | C | TRP | B | 109 | 22.949 | 9.473 | −5.831 | 1.00 | 26.27 | C |
| ATOM | 2781 | O | TRP | B | 109 | 22.277 | 8.576 | −5.328 | 1.00 | 28.84 | O |
| ATOM | 2782 | CB | TRP | B | 109 | 23.642 | 9.496 | −8.259 | 1.00 | 20.36 | C |
| ATOM | 2783 | CG | TRP | B | 109 | 23.961 | 8.020 | −8.161 | 1.00 | 21.75 | C |
| ATOM | 2784 | CD1 | TRP | B | 109 | 23.114 | 6.977 | −8.415 | 1.00 | 23.62 | C |
| ATOM | 2785 | CD2 | TRP | B | 109 | 25.212 | 7.433 | −7.779 | 1.00 | 17.80 | C |
| ATOM | 2786 | NE1 | TRP | B | 109 | 23.756 | 5.782 | −8.210 | 1.00 | 19.75 | N |
| ATOM | 2787 | CE2 | TRP | B | 109 | 25.044 | 6.031 | −7.817 | 1.00 | 20.67 | C |
| ATOM | 2788 | CE3 | TRP | B | 109 | 26.454 | 7.954 | −7.410 | 1.00 | 24.00 | C |
| ATOM | 2789 | CZ2 | TRP | B | 109 | 26.078 | 5.141 | −7.498 | 1.00 | 19.81 | C |
| ATOM | 2790 | CZ3 | TRP | B | 109 | 27.480 | 7.071 | −7.094 | 1.00 | 27.21 | C |
| ATOM | 2791 | CH2 | TRP | B | 109 | 27.283 | 5.681 | −7.140 | 1.00 | 23.73 | C |
| ATOM | 2792 | N | THR | B | 110 | 23.986 | 10.042 | −5.222 | 1.00 | 24.38 | N |
| ATOM | 2793 | CA | THR | B | 110 | 24.449 | 9.634 | −3.904 | 1.00 | 24.83 | C |
| ATOM | 2794 | C | THR | B | 110 | 23.351 | 9.824 | −2.853 | 1.00 | 23.81 | C |
| ATOM | 2795 | O | THR | B | 110 | 23.055 | 8.905 | −2.109 | 1.00 | 27.17 | O |
| ATOM | 2796 | CB | THR | B | 110 | 25.739 | 10.410 | −3.509 | 1.00 | 25.69 | C |
| ATOM | 2797 | OG1 | THR | B | 110 | 26.793 | 10.058 | −4.412 | 1.00 | 27.99 | O |
| ATOM | 2798 | CG2 | THR | B | 110 | 26.180 | 10.093 | −2.075 | 1.00 | 14.32 | C |
| ATOM | 2799 | N | SER | B | 111 | 22.758 | 11.014 | −2.799 | 1.00 | 23.96 | N |
| ATOM | 2800 | CA | SER | B | 111 | 21.563 | 11.270 | −1.994 | 1.00 | 24.92 | C |
| ATOM | 2801 | C | SER | B | 111 | 20.506 | 10.156 | −2.099 | 1.00 | 32.13 | C |
| ATOM | 2802 | O | SER | B | 111 | 19.967 | 9.699 | −1.075 | 1.00 | 21.22 | O |
| ATOM | 2803 | CB | SER | B | 111 | 20.913 | 12.586 | −2.419 | 1.00 | 25.11 | C |
| ATOM | 2804 | OG | SER | B | 111 | 21.713 | 13.694 | −2.058 | 1.00 | 35.53 | O |
| ATOM | 2805 | N | LEU | B | 112 | 20.210 | 9.737 | −3.334 | 1.00 | 20.40 | N |
| ATOM | 2806 | CA | LEU | B | 112 | 19.181 | 8.732 | −3.604 | 1.00 | 22.72 | C |
| ATOM | 2807 | C | LEU | B | 112 | 19.499 | 7.368 | −3.055 | 1.00 | 24.74 | C |
| ATOM | 2808 | O | LEU | B | 112 | 18.628 | 6.656 | −2.554 | 1.00 | 27.64 | O |
| ATOM | 2809 | CB | LEU | B | 112 | 18.983 | 8.572 | −5.096 | 1.00 | 21.13 | C |
| ATOM | 2810 | CG | LEU | B | 112 | 17.857 | 9.423 | −5.637 | 1.00 | 35.52 | C |
| ATOM | 2811 | CD1 | LEU | B | 112 | 17.523 | 8.949 | −7.045 | 1.00 | 28.77 | C |
| ATOM | 2812 | CD2 | LEU | B | 112 | 16.651 | 9.337 | −4.685 | 1.00 | 23.06 | C |
| ATOM | 2813 | N | ASP | B | 113 | 20.755 | 7.001 | −3.201 | 1.00 | 19.58 | N |
| ATOM | 2814 | CA | ASP | B | 113 | 21.250 | 5.717 | −2.774 | 1.00 | 26.51 | C |
| ATOM | 2815 | C | ASP | B | 113 | 21.042 | 5.564 | −1.269 | 1.00 | 26.23 | C |
| ATOM | 2816 | O | ASP | B | 113 | 20.491 | 4.569 | −0.800 | 1.00 | 27.54 | O |
| ATOM | 2817 | CB | ASP | B | 113 | 22.735 | 5.670 | −3.128 | 1.00 | 30.13 | C |
| ATOM | 2818 | CG | ASP | B | 113 | 23.356 | 4.330 | −2.892 | 1.00 | 28.63 | C |
| ATOM | 2819 | OD1 | ASP | B | 113 | 22.804 | 3.531 | −2.105 | 1.00 | 31.60 | O |
| ATOM | 2820 | OD2 | ASP | B | 113 | 24.416 | 4.092 | −3.493 | 1.00 | 28.09 | O |
| ATOM | 2821 | N | VAL | B | 114 | 21.479 | 6.581 | −0.538 | 1.00 | 21.39 | N |
| ATOM | 2822 | CA | VAL | B | 114 | 21.434 | 6.623 | 0.918 | 1.00 | 24.98 | C |
| ATOM | 2823 | C | VAL | B | 114 | 20.007 | 6.675 | 1.462 | 1.00 | 24.83 | C |
| ATOM | 2824 | O | VAL | B | 114 | 19.663 | 5.980 | 2.430 | 1.00 | 21.53 | O |
| ATOM | 2825 | CB | VAL | B | 114 | 22.258 | 7.832 | 1.434 | 1.00 | 28.12 | C |
| ATOM | 2826 | CG1 | VAL | B | 114 | 22.135 | 7.984 | 2.922 | 1.00 | 23.27 | C |
| ATOM | 2827 | CG2 | VAL | B | 114 | 23.720 | 7.646 | 1.069 | 1.00 | 28.64 | C |
| ATOM | 2828 | N | LEU | B | 115 | 19.181 | 7.503 | 0.834 | 1.00 | 26.86 | N |
| ATOM | 2829 | CA | LEU | B | 115 | 17.753 | 7.572 | 1.151 | 1.00 | 20.55 | C |
| ATOM | 2830 | C | LEU | B | 115 | 17.083 | 6.205 | 1.074 | 1.00 | 23.52 | C |
| ATOM | 2831 | O | LEU | B | 115 | 16.332 | 5.831 | 1.977 | 1.00 | 24.11 | O |
| ATOM | 2832 | CB | LEU | B | 115 | 17.042 | 8.539 | 0.206 | 1.00 | 19.11 | C |
| ATOM | 2833 | CG | LEU | B | 115 | 15.553 | 8.698 | 0.472 | 1.00 | 22.12 | C |
| ATOM | 2834 | CD1 | LEU | B | 115 | 15.321 | 9.013 | 1.935 | 1.00 | 21.95 | C |
| ATOM | 2835 | CD2 | LEU | B | 115 | 15.012 | 9.793 | −0.405 | 1.00 | 21.91 | C |
| ATOM | 2836 | N | CYS | B | 116 | 17.360 | 5.459 | 0.002 | 1.00 | 22.70 | N |
| ATOM | 2837 | CA | CYS | B | 116 | 16.734 | 4.147 | −0.183 | 1.00 | 31.84 | C |
| ATOM | 2838 | C | CYS | B | 116 | 17.123 | 3.104 | 0.878 | 1.00 | 32.18 | C |
| ATOM | 2839 | O | CYS | B | 116 | 16.287 | 2.302 | 1.287 | 1.00 | 32.32 | O |
| ATOM | 2840 | CB | CYS | B | 116 | 16.966 | 3.600 | −1.596 | 1.00 | 20.17 | C |
| ATOM | 2841 | SG | CYS | B | 116 | 16.019 | 4.461 | −2.846 | 1.00 | 40.86 | S |
| ATOM | 2842 | N | VAL | B | 117 | 18.374 | 3.116 | 1.326 | 1.00 | 22.95 | N |
| ATOM | 2843 | CA | VAL | B | 117 | 18.794 | 2.187 | 2.372 | 1.00 | 31.86 | C |
| ATOM | 2844 | C | VAL | B | 117 | 18.292 | 2.616 | 3.762 | 1.00 | 35.14 | C |
| ATOM | 2845 | O | VAL | B | 117 | 17.927 | 1.769 | 4.584 | 1.00 | 23.25 | O |
| ATOM | 2846 | CB | VAL | B | 117 | 20.329 | 1.985 | 2.404 | 1.00 | 27.89 | C |
| ATOM | 2847 | CG1 | VAL | B | 117 | 20.681 | 0.799 | 3.287 | 1.00 | 29.36 | C |
| ATOM | 2848 | CG2 | VAL | B | 117 | 20.856 | 1.765 | 1.016 | 1.00 | 23.68 | C |
| ATOM | 2849 | N | THR | B | 118 | 18.274 | 3.923 | 4.024 | 1.00 | 29.96 | N |
| ATOM | 2850 | CA | THR | B | 118 | 17.734 | 4.412 | 5.285 | 1.00 | 30.83 | C |
| ATOM | 2851 | C | THR | B | 118 | 16.255 | 4.048 | 5.408 | 1.00 | 29.14 | C |
| ATOM | 2852 | O | THR | B | 118 | 15.827 | 3.541 | 6.444 | 1.00 | 22.53 | O |
| ATOM | 2853 | CB | THR | B | 118 | 17.888 | 5.943 | 5.437 | 1.00 | 33.94 | C |
| ATOM | 2854 | OG1 | THR | B | 118 | 19.275 | 6.298 | 5.415 | 1.00 | 28.33 | O |
| ATOM | 2855 | CG2 | THR | B | 118 | 17.262 | 6.423 | 6.745 | 1.00 | 22.28 | C |
| ATOM | 2856 | N | ALA | B | 119 | 15.487 | 4.300 | 4.345 | 1.00 | 23.77 | N |
| ATOM | 2857 | CA | ALA | B | 119 | 14.044 | 4.043 | 4.366 | 1.00 | 23.58 | C |
| ATOM | 2858 | C | ALA | B | 119 | 13.688 | 2.549 | 4.490 | 1.00 | 23.51 | C |
| ATOM | 2859 | O | ALA | B | 119 | 12.684 | 2.195 | 5.091 | 1.00 | 27.98 | O |
| ATOM | 2860 | CB | ALA | B | 119 | 13.354 | 4.686 | 3.168 | 1.00 | 21.64 | C |
| ATOM | 2861 | N | SER | B | 120 | 14.522 | 1.676 | 3.944 | 1.00 | 27.68 | N |
| ATOM | 2862 | CA | SER | B | 120 | 14.299 | 0.236 | 4.063 | 1.00 | 29.85 | C |
| ATOM | 2863 | C | SER | B | 120 | 14.371 | −0.213 | 5.515 | 1.00 | 27.06 | C |
| ATOM | 2864 | O | SER | B | 120 | 13.384 | −0.693 | 6.063 | 1.00 | 30.48 | O |
| ATOM | 2865 | CB | SER | B | 120 | 15.312 | −0.569 | 3.237 | 1.00 | 24.87 | C |
| ATOM | 2866 | OG | SER | B | 120 | 15.134 | −0.371 | 1.853 | 1.00 | 35.56 | O |
| ATOM | 2867 | N | ILE | B | 121 | 15.548 | −0.068 | 6.121 | 1.00 | 22.70 | N |
| ATOM | 2868 | CA | ILE | B | 121 | 15.761 | −0.487 | 7.499 | 1.00 | 26.14 | C |
| ATOM | 2869 | C | ILE | B | 121 | 14.727 | 0.192 | 8.410 | 1.00 | 27.72 | C |
| ATOM | 2870 | O | ILE | B | 121 | 14.164 | −0.438 | 9.298 | 1.00 | 27.02 | O |
| ATOM | 2871 | CB | ILE | B | 121 | 17.222 | −0.224 | 7.978 | 1.00 | 21.40 | C |
| ATOM | 2872 | CG1 | ILE | B | 121 | 17.555 | −1.041 | 9.228 | 1.00 | 23.57 | C |
| ATOM | 2873 | CG2 | ILE | B | 121 | 17.447 | 1.247 | 8.259 | 1.00 | 20.63 | C |
| ATOM | 2874 | CD1 | ILE | B | 121 | 17.120 | −2.472 | 9.164 | 1.00 | 30.47 | C |
| ATOM | 2875 | N | GLU | B | 122 | 14.452 | 1.468 | 8.163 | 1.00 | 32.55 | N |
| ATOM | 2876 | CA | GLU | B | 122 | 13.445 | 2.173 | 8.949 | 1.00 | 29.38 | C |
| ATOM | 2877 | C | GLU | B | 122 | 12.087 | 1.514 | 8.816 | 1.00 | 29.68 | C |
| ATOM | 2878 | O | GLU | B | 122 | 11.362 | 1.392 | 9.800 | 1.00 | 28.19 | O |
| ATOM | 2879 | CB | GLU | B | 122 | 13.348 | 3.647 | 8.552 | 1.00 | 27.52 | C |
| ATOM | 2880 | CG | GLU | B | 122 | 14.275 | 4.565 | 9.335 | 1.00 | 28.18 | C |
| ATOM | 2881 | CD | GLU | B | 122 | 13.991 | 6.044 | 9.071 | 1.00 | 42.72 | C |
| ATOM | 2882 | OE1 | GLU | B | 122 | 12.911 | 6.362 | 8.528 | 1.00 | 39.95 | O |
| ATOM | 2883 | OE2 | GLU | B | 122 | 14.845 | 6.895 | 9.411 | 1.00 | 45.99 | O |
| ATOM | 2884 | N | THR | B | 123 | 11.741 | 1.095 | 7.599 | 1.00 | 32.57 | N |
| ATOM | 2885 | CA | THR | B | 123 | 10.471 | 0.414 | 7.361 | 1.00 | 28.73 | C |
| ATOM | 2886 | C | THR | B | 123 | 10.455 | −0.975 | 7.999 | 1.00 | 32.15 | C |
| ATOM | 2887 | O | THR | B | 123 | 9.474 | −1.368 | 8.614 | 1.00 | 31.18 | O |
| ATOM | 2888 | CB | THR | B | 123 | 10.154 | 0.304 | 5.866 | 1.00 | 28.74 | C |
| ATOM | 2889 | OG1 | THR | B | 123 | 9.810 | 1.596 | 5.364 | 1.00 | 32.13 | O |
| ATOM | 2890 | CG2 | THR | B | 123 | 8.988 | −0.638 | 5.632 | 1.00 | 29.92 | C |
| ATOM | 2891 | N | LEU | B | 124 | 11.544 | −1.718 | 7.858 | 1.00 | 30.78 | N |
| ATOM | 2892 | CA | LEU | B | 124 | 11.631 | −3.025 | 8.488 | 1.00 | 30.84 | C |
| ATOM | 2893 | C | LEU | B | 124 | 11.421 | −2.893 | 9.989 | 1.00 | 33.17 | C |
| ATOM | 2894 | O | LEU | B | 124 | 10.918 | −3.797 | 10.651 | 1.00 | 39.00 | O |
| ATOM | 2895 | CB | LEU | B | 124 | 12.983 | −3.668 | 8.183 | 1.00 | 33.47 | C |
| ATOM | 2896 | CG | LEU | B | 124 | 13.043 | −4.233 | 6.759 | 1.00 | 28.66 | C |
| ATOM | 2897 | CD1 | LEU | B | 124 | 14.462 | −4.628 | 6.351 | 1.00 | 29.17 | C |
| ATOM | 2898 | CD2 | LEU | B | 124 | 12.105 | −5.410 | 6.670 | 1.00 | 32.69 | C |
| ATOM | 2899 | N | CYS | B | 125 | 11.806 | −1.749 | 10.526 | 1.00 | 35.71 | N |
| ATOM | 2900 | CA | CYS | B | 125 | 11.635 | −1.491 | 11.943 | 1.00 | 39.83 | C |
| ATOM | 2901 | C | CYS | B | 125 | 10.146 | −1.432 | 12.249 | 1.00 | 36.88 | C |
| ATOM | 2902 | O | CYS | B | 125 | 9.635 | −2.169 | 13.086 | 1.00 | 36.27 | O |
| ATOM | 2903 | CB | CYS | B | 125 | 12.286 | −0.162 | 12.303 | 1.00 | 36.68 | C |
| ATOM | 2904 | SG | CYS | B | 125 | 13.359 | −0.278 | 13.695 | 1.00 | 49.77 | S |
| ATOM | 2905 | N | VAL | B | 126 | 9.455 | −0.546 | 11.546 | 1.00 | 31.52 | N |
| ATOM | 2906 | CA | VAL | B | 126 | 8.025 | −0.407 | 11.705 | 1.00 | 28.31 | C |
| ATOM | 2907 | C | VAL | B | 126 | 7.307 | −1.753 | 11.580 | 1.00 | 32.71 | C |
| ATOM | 2908 | O | VAL | B | 126 | 6.496 | −2.086 | 12.431 | 1.00 | 43.13 | O |
| ATOM | 2909 | CB | VAL | B | 126 | 7.464 | 0.644 | 10.741 | 1.00 | 29.20 | C |
| ATOM | 2910 | CG1 | VAL | B | 126 | 5.944 | 0.555 | 10.653 | 1.00 | 32.82 | C |
| ATOM | 2911 | CG2 | VAL | B | 126 | 7.905 | 2.024 | 11.184 | 1.00 | 22.76 | C |
| ATOM | 2912 | N | ILE | B | 127 | 7.617 | −2.531 | 10.547 | 1.00 | 30.89 | N |
| ATOM | 2913 | CA | ILE | B | 127 | 7.056 | −3.877 | 10.391 | 1.00 | 34.33 | C |
| ATOM | 2914 | C | ILE | B | 127 | 7.192 | −4.711 | 11.672 | 1.00 | 41.02 | C |
| ATOM | 2915 | O | ILE | B | 127 | 6.238 | −5.365 | 12.099 | 1.00 | 43.20 | O |
| ATOM | 2916 | CB | ILE | B | 127 | 7.737 | −4.664 | 9.231 | 1.00 | 49.96 | C |
| ATOM | 2917 | CG1 | ILE | B | 127 | 7.315 | −4.127 | 7.861 | 1.00 | 35.86 | C |
| ATOM | 2918 | CG2 | ILE | B | 127 | 7.434 | −6.174 | 9.316 | 1.00 | 35.81 | C |
| ATOM | 2919 | CD1 | ILE | B | 127 | 8.134 | −4.729 | 6.737 | 1.00 | 36.93 | C |
| ATOM | 2920 | N | ALA | B | 128 | 8.370 | −4.697 | 12.287 | 1.00 | 29.58 | N |
| ATOM | 2921 | CA | ALA | B | 128 | 8.588 | −5.494 | 13.491 | 1.00 | 31.62 | C |
| ATOM | 2922 | C | ALA | B | 128 | 7.773 | −4.984 | 14.675 | 1.00 | 39.85 | C |
| ATOM | 2923 | O | ALA | B | 128 | 7.077 | −5.754 | 15.331 | 1.00 | 49.30 | O |
| ATOM | 2924 | CB | ALA | B | 128 | 10.067 | −5.546 | 13.843 | 1.00 | 37.51 | C |
| ATOM | 2925 | N | ILE | B | 129 | 7.866 | −3.685 | 14.942 | 1.00 | 35.48 | N |
| ATOM | 2926 | CA | ILE | B | 129 | 7.127 | −3.052 | 16.026 | 1.00 | 35.81 | C |
| ATOM | 2927 | C | ILE | B | 129 | 5.615 | −3.220 | 15.875 | 1.00 | 38.43 | C |
| ATOM | 2928 | O | ILE | B | 129 | 4.896 | −3.353 | 16.859 | 1.00 | 39.36 | O |
| ATOM | 2929 | CB | ILE | B | 129 | 7.440 | −1.547 | 16.100 | 1.00 | 42.07 | C |
| ATOM | 2930 | CG1 | ILE | B | 129 | 8.877 | −1.327 | 16.574 | 1.00 | 36.46 | C |
| ATOM | 2931 | CG2 | ILE | B | 129 | 6.432 | −0.823 | 17.008 | 1.00 | 29.72 | C |
| ATOM | 2932 | CD1 | ILE | B | 129 | 9.311 | 0.115 | 16.497 | 1.00 | 29.25 | C |
| ATOM | 2933 | N | ASP | B | 130 | 5.143 | −3.191 | 14.635 | 1.00 | 38.72 | N |
| ATOM | 2934 | CA | ASP | B | 130 | 3.730 | −3.367 | 14.319 | 1.00 | 37.47 | C |
| ATOM | 2935 | C | ASP | B | 130 | 3.277 | −4.763 | 14.711 | 1.00 | 39.01 | C |
| ATOM | 2936 | O | ASP | B | 130 | 2.281 | −4.927 | 15.403 | 1.00 | 36.78 | O |
| ATOM | 2937 | CB | ASP | B | 130 | 3.507 | −3.124 | 12.819 | 1.00 | 38.15 | C |
| ATOM | 2938 | CG | ASP | B | 130 | 2.348 | −3.933 | 12.245 | 1.00 | 53.55 | C |
| ATOM | 2939 | OD1 | ASP | B | 130 | 1.202 | −3.716 | 12.688 | 1.00 | 64.40 | O |
| ATOM | 2940 | OD2 | ASP | B | 130 | 2.576 | −4.768 | 11.331 | 1.00 | 54.68 | O |
| ATOM | 2941 | N | ARG | B | 131 | 4.022 | −5.764 | 14.255 | 1.00 | 41.77 | N |
| ATOM | 2942 | CA | ARG | B | 131 | 3.760 | −7.151 | 14.602 | 1.00 | 45.38 | C |
| ATOM | 2943 | C | ARG | B | 131 | 3.791 | −7.350 | 16.113 | 1.00 | 41.25 | C |
| ATOM | 2944 | O | ARG | B | 131 | 2.918 | −8.001 | 16.677 | 1.00 | 36.53 | O |
| ATOM | 2945 | CB | ARG | B | 131 | 4.791 | −8.067 | 13.933 | 1.00 | 42.90 | C |
| ATOM | 2946 | CG | ARG | B | 131 | 4.585 | −8.251 | 12.438 | 1.00 | 41.24 | C |
| ATOM | 2947 | CD | ARG | B | 131 | 3.193 | −8.765 | 12.122 | 1.00 | 40.05 | C |
| ATOM | 2948 | NE | ARG | B | 131 | 2.207 | −7.692 | 12.047 | 1.00 | 51.13 | N |
| ATOM | 2949 | CZ | ARG | B | 131 | 0.895 | −7.889 | 11.964 | 1.00 | 57.15 | C |
| ATOM | 2950 | NH1 | ARG | B | 131 | 0.410 | −9.128 | 11.954 | 1.00 | 59.37 | N |
| ATOM | 2951 | NH2 | ARG | B | 131 | 0.067 | −6.852 | 11.899 | 1.00 | 42.60 | N |
| ATOM | 2952 | N | TYR | B | 132 | 4.806 | −6.793 | 16.764 | 1.00 | 35.87 | N |
| ATOM | 2953 | CA | TYR | B | 132 | 4.923 | −6.935 | 18.204 | 1.00 | 47.68 | C |
| ATOM | 2954 | C | TYR | B | 132 | 3.740 | −6.333 | 18.970 | 1.00 | 51.85 | C |
| ATOM | 2955 | O | TYR | B | 132 | 3.397 | −6.803 | 20.053 | 1.00 | 53.14 | O |
| ATOM | 2956 | CB | TYR | B | 132 | 6.229 | −6.337 | 18.720 | 1.00 | 48.84 | C |
| ATOM | 2957 | CG | TYR | B | 132 | 6.307 | −6.352 | 20.224 | 1.00 | 41.78 | C |
| ATOM | 2958 | CD1 | TYR | B | 132 | 6.784 | −7.463 | 20.901 | 1.00 | 49.77 | C |
| ATOM | 2959 | CD2 | TYR | B | 132 | 5.878 | −5.263 | 20.965 | 1.00 | 50.94 | C |
| ATOM | 2960 | CE1 | TYR | B | 132 | 6.843 | −7.484 | 22.274 | 1.00 | 53.70 | C |
| ATOM | 2961 | CE2 | TYR | B | 132 | 5.938 | −5.271 | 22.332 | 1.00 | 57.21 | C |
| ATOM | 2962 | CZ | TYR | B | 132 | 6.418 | −6.385 | 22.982 | 1.00 | 58.95 | C |
| ATOM | 2963 | OH | TYR | B | 132 | 6.467 | −6.393 | 24.352 | 1.00 | 72.24 | O |
| ATOM | 2964 | N | LEU | B | 133 | 3.130 | −5.288 | 18.423 | 1.00 | 42.80 | N |
| ATOM | 2965 | CA | LEU | B | 133 | 1.929 | −4.725 | 19.020 | 1.00 | 39.15 | C |
| ATOM | 2966 | C | LEU | B | 133 | 0.697 | −5.587 | 18.722 | 1.00 | 44.17 | C |
| ATOM | 2967 | O | LEU | B | 133 | −0.135 | −5.819 | 19.595 | 1.00 | 47.92 | O |
| ATOM | 2968 | CB | LEU | B | 133 | 1.698 | −3.292 | 18.541 | 1.00 | 32.41 | C |
| ATOM | 2969 | CG | LEU | B | 133 | 2.715 | −2.218 | 18.928 | 1.00 | 37.90 | C |
| ATOM | 2970 | CD1 | LEU | B | 133 | 2.348 | −0.900 | 18.249 | 1.00 | 30.23 | C |
| ATOM | 2971 | CD2 | LEU | B | 133 | 2.825 | −2.032 | 20.443 | 1.00 | 32.12 | C |
| ATOM | 2972 | N | ALA | B | 134 | 0.578 | −6.059 | 17.488 | 1.00 | 46.03 | N |
| ATOM | 2973 | CA | ALA | B | 134 | −0.567 | −6.869 | 17.107 | 1.00 | 43.43 | C |
| ATOM | 2974 | C | ALA | B | 134 | −0.584 | −8.115 | 17.963 | 1.00 | 42.33 | C |
| ATOM | 2975 | O | ALA | B | 134 | −1.630 | −8.554 | 18.420 | 1.00 | 51.73 | O |
| ATOM | 2976 | CB | ALA | B | 134 | −0.513 | −7.230 | 15.628 | 1.00 | 26.95 | C |
| ATOM | 2977 | N | ILE | B | 135 | 0.594 | −8.666 | 18.207 | 1.00 | 52.56 | N |
| ATOM | 2978 | CA | ILE | B | 135 | 0.699 | −9.939 | 18.899 | 1.00 | 57.12 | C |
| ATOM | 2979 | C | ILE | B | 135 | 0.463 | −9.812 | 20.410 | 1.00 | 53.25 | C |
| ATOM | 2980 | O | ILE | B | 135 | −0.121 | −10.708 | 21.016 | 1.00 | 58.54 | O |
| ATOM | 2981 | CB | ILE | B | 135 | 2.055 | −10.614 | 18.619 | 1.00 | 59.12 | C |
| ATOM | 2982 | CG1 | ILE | B | 135 | 1.856 | −12.039 | 18.118 | 1.00 | 50.85 | C |
| ATOM | 2983 | CG2 | ILE | B | 135 | 2.945 | −10.578 | 19.856 | 1.00 | 66.10 | C |
| ATOM | 2984 | CD1 | ILE | B | 135 | 3.163 | −12.765 | 17.882 | 1.00 | 65.69 | C |
| ATOM | 2985 | N | THR | B | 136 | 0.900 | −8.707 | 21.015 | 1.00 | 51.61 | N |
| ATOM | 2986 | CA | THR | B | 136 | 0.699 | −8.508 | 22.459 | 1.00 | 47.78 | C |
| ATOM | 2987 | C | THR | B | 136 | −0.581 | −7.740 | 22.824 | 1.00 | 54.84 | C |
| ATOM | 2988 | O | THR | B | 136 | −1.180 | −8.015 | 23.858 | 1.00 | 52.93 | O |
| ATOM | 2989 | CB | THR | B | 136 | 1.896 | −7.802 | 23.159 | 1.00 | 52.23 | C |
| ATOM | 2990 | OG1 | THR | B | 136 | 1.894 | −6.400 | 22.842 | 1.00 | 46.45 | O |
| ATOM | 2991 | CG2 | THR | B | 136 | 3.233 | −8.448 | 22.779 | 1.00 | 46.53 | C |
| ATOM | 2992 | N | SER | B | 137 | −0.997 | −6.784 | 21.992 | 1.00 | 57.43 | N |
| ATOM | 2993 | CA | SER | B | 137 | −2.157 | −5.942 | 22.319 | 1.00 | 47.89 | C |
| ATOM | 2994 | C | SER | B | 137 | −3.302 | −5.990 | 21.310 | 1.00 | 48.29 | C |
| ATOM | 2995 | O | SER | B | 137 | −3.835 | −4.945 | 20.933 | 1.00 | 49.37 | O |
| ATOM | 2996 | CB | SER | B | 137 | −1.726 | −4.484 | 22.483 | 1.00 | 44.38 | C |
| ATOM | 2997 | OG | SER | B | 137 | −0.867 | −4.324 | 23.591 | 1.00 | 60.02 | O |
| ATOM | 2998 | N | PRO | B | 138 | −3.708 | −7.197 | 20.892 | 1.00 | 52.06 | N |
| ATOM | 2999 | CA | PRO | B | 138 | −4.699 | −7.358 | 19.818 | 1.00 | 57.37 | C |
| ATOM | 3000 | C | PRO | B | 138 | −5.893 | −6.401 | 19.873 | 1.00 | 59.27 | C |
| ATOM | 3001 | O | PRO | B | 138 | −6.288 | −5.894 | 18.823 | 1.00 | 65.54 | O |
| ATOM | 3002 | CB | PRO | B | 138 | −5.163 | −8.812 | 19.990 | 1.00 | 40.41 | C |
| ATOM | 3003 | CG | PRO | B | 138 | −4.621 | −9.230 | 21.335 | 1.00 | 57.14 | C |
| ATOM | 3004 | CD | PRO | B | 138 | −3.337 | −8.500 | 21.450 | 1.00 | 40.00 | C |
| ATOM | 3005 | N | PHE | B | 139 | −6.459 | −6.161 | 21.053 | 1.00 | 61.30 | N |
| ATOM | 3006 | CA | PHE | B | 139 | −7.629 | −5.287 | 21.158 | 1.00 | 62.66 | C |
| ATOM | 3007 | C | PHE | B | 139 | −7.265 | −3.812 | 21.004 | 1.00 | 57.87 | C |
| ATOM | 3008 | O | PHE | B | 139 | −7.909 | −3.086 | 20.248 | 1.00 | 67.75 | O |
| ATOM | 3009 | CB | PHE | B | 139 | −8.394 | −5.514 | 22.469 | 1.00 | 83.75 | C |
| ATOM | 3010 | CG | PHE | B | 139 | −9.665 | −4.707 | 22.578 | 1.00 | 92.58 | C |
| ATOM | 3011 | CD1 | PHE | B | 139 | −10.861 | −5.200 | 22.078 | 1.00 | 91.86 | C |
| ATOM | 3012 | CD2 | PHE | B | 139 | −9.660 | −3.449 | 23.168 | 1.00 | 85.95 | C |
| ATOM | 3013 | CE1 | PHE | B | 139 | −12.028 | −4.455 | 22.167 | 1.00 | 86.07 | C |
| ATOM | 3014 | CE2 | PHE | B | 139 | −10.823 | −2.701 | 23.258 | 1.00 | 81.86 | C |
| ATOM | 3015 | CZ | PHE | B | 139 | −12.006 | −3.205 | 22.757 | 1.00 | 82.49 | C |
| ATOM | 3016 | N | ARG | B | 140 | −6.239 | −3.366 | 21.715 | 1.00 | 52.27 | N |
| ATOM | 3017 | CA | ARG | B | 140 | −5.794 | −1.983 | 21.589 | 1.00 | 57.00 | C |
| ATOM | 3018 | C | ARG | B | 140 | −5.163 | −1.719 | 20.228 | 1.00 | 58.08 | C |
| ATOM | 3019 | O | ARG | B | 140 | −4.957 | −0.572 | 19.843 | 1.00 | 67.10 | O |
| ATOM | 3020 | CB | ARG | B | 140 | −4.826 | −1.606 | 22.708 | 1.00 | 55.01 | C |
| ATOM | 3021 | CG | ARG | B | 140 | −5.506 | −1.336 | 24.037 | 1.00 | 58.27 | C |
| ATOM | 3022 | CD | ARG | B | 140 | −4.518 | −0.792 | 25.051 | 1.00 | 73.82 | C |
| ATOM | 3023 | NE | ARG | B | 140 | −5.188 | −0.319 | 26.259 | 1.00 | 88.96 | N |
| ATOM | 3024 | CZ | ARG | B | 140 | −4.581 | 0.361 | 27.226 | 1.00 | 91.96 | C |
| ATOM | 3025 | NH1 | ARG | B | 140 | −3.289 | 0.646 | 27.121 | 1.00 | 91.58 | N |
| ATOM | 3026 | NH2 | ARG | B | 140 | −5.261 | 0.760 | 28.293 | 1.00 | 83.70 | N |
| ATOM | 3027 | N | TYR | B | 141 | −4.870 | −2.784 | 19.494 | 1.00 | 55.37 | N |
| ATOM | 3028 | CA | TYR | B | 141 | −4.294 | −2.655 | 18.160 | 1.00 | 50.90 | C |
| ATOM | 3029 | C | TYR | B | 141 | −5.333 | −2.402 | 17.060 | 1.00 | 61.21 | C |
| ATOM | 3030 | O | TYR | B | 141 | −5.179 | −1.470 | 16.272 | 1.00 | 60.06 | O |
| ATOM | 3031 | CB | TYR | B | 141 | −3.452 | −3.884 | 17.826 | 1.00 | 53.15 | C |
| ATOM | 3032 | CG | TYR | B | 141 | −2.849 | −3.861 | 16.443 | 1.00 | 52.99 | C |
| ATOM | 3033 | CD1 | TYR | B | 141 | −1.631 | −3.239 | 16.200 | 1.00 | 57.12 | C |
| ATOM | 3034 | CD2 | TYR | B | 141 | −3.502 | −4.463 | 15.379 | 1.00 | 60.99 | C |
| ATOM | 3035 | CE1 | TYR | B | 141 | −1.085 | −3.220 | 14.926 | 1.00 | 59.63 | C |
| ATOM | 3036 | CE2 | TYR | B | 141 | −2.969 | −4.449 | 14.113 | 1.00 | 58.95 | C |
| ATOM | 3037 | CZ | TYR | B | 141 | −1.762 | −3.831 | 13.885 | 1.00 | 57.45 | C |
| ATOM | 3038 | OH | TYR | B | 141 | −1.245 | −3.831 | 12.608 | 1.00 | 57.25 | O |
| ATOM | 3039 | N | GLN | B | 142 | −6.379 | −3.230 | 17.009 | 1.00 | 64.26 | N |
| ATOM | 3040 | CA | GLN | B | 142 | −7.442 | −3.109 | 16.002 | 1.00 | 70.94 | C |
| ATOM | 3041 | C | GLN | B | 142 | −8.073 | −1.716 | 15.917 | 1.00 | 73.96 | C |
| ATOM | 3042 | O | GLN | B | 142 | −8.322 | −1.197 | 14.822 | 1.00 | 72.85 | O |
| ATOM | 3043 | CB | GLN | B | 142 | −8.560 | −4.107 | 16.285 | 1.00 | 87.97 | C |
| ATOM | 3044 | CG | GLN | B | 142 | −8.303 | −5.527 | 15.838 | 1.00 | 97.92 | C |
| ATOM | 3045 | CD | GLN | B | 142 | −9.432 | −6.457 | 16.254 | 1.00 | 121.46 | C |
| ATOM | 3046 | OE1 | GLN | B | 142 | −10.584 | −6.030 | 16.412 | 1.00 | 102.41 | O |
| ATOM | 3047 | NE2 | GLN | B | 142 | −9.106 | −7.732 | 16.445 | 1.00 | 119.33 | N |
| ATOM | 3048 | N | SER | B | 143 | −8.353 | −1.128 | 17.076 | 1.00 | 67.38 | N |
| ATOM | 3049 | CA | SER | B | 143 | −9.030 | 0.164 | 17.131 | 1.00 | 68.27 | C |
| ATOM | 3050 | C | SER | B | 143 | −8.125 | 1.315 | 16.702 | 1.00 | 71.56 | C |
| ATOM | 3051 | O | SER | B | 143 | −8.559 | 2.212 | 15.979 | 1.00 | 75.84 | O |
| ATOM | 3052 | CB | SER | B | 143 | −9.631 | 0.417 | 18.526 | 1.00 | 78.89 | C |
| ATOM | 3053 | OG | SER | B | 143 | −8.974 | −0.337 | 19.535 | 1.00 | 70.07 | O |
| ATOM | 3054 | N | LEU | B | 144 | −6.866 | 1.279 | 17.132 | 1.00 | 62.02 | N |
| ATOM | 3055 | CA | LEU | B | 144 | −5.927 | 2.344 | 16.802 | 1.00 | 60.71 | C |
| ATOM | 3056 | C | LEU | B | 144 | −5.374 | 2.267 | 15.373 | 1.00 | 61.44 | C |
| ATOM | 3057 | O | LEU | B | 144 | −5.156 | 3.293 | 14.729 | 1.00 | 60.22 | O |
| ATOM | 3058 | CB | LEU | B | 144 | −4.796 | 2.398 | 17.831 | 1.00 | 56.74 | C |
| ATOM | 3059 | CG | LEU | B | 144 | −5.348 | 2.731 | 19.215 | 1.00 | 66.86 | C |
| ATOM | 3060 | CD1 | LEU | B | 144 | −4.256 | 3.170 | 20.178 | 1.00 | 46.16 | C |
| ATOM | 3061 | CD2 | LEU | B | 144 | −6.413 | 3.808 | 19.076 | 1.00 | 57.79 | C |
| ATOM | 3062 | N | MET | B | 145 | −5.159 | 1.060 | 14.868 | 1.00 | 52.98 | N |
| ATOM | 3063 | CA | MET | B | 145 | −4.512 | 0.908 | 13.568 | 1.00 | 60.61 | C |
| ATOM | 3064 | C | MET | B | 145 | −5.464 | 0.693 | 12.388 | 1.00 | 53.88 | C |
| ATOM | 3065 | O | MET | B | 145 | −5.989 | −0.396 | 12.198 | 1.00 | 54.51 | O |
| ATOM | 3066 | CB | MET | B | 145 | −3.470 | −0.214 | 13.624 | 1.00 | 57.45 | C |
| ATOM | 3067 | CG | MET | B | 145 | −2.083 | 0.241 | 14.073 | 1.00 | 69.20 | C |
| ATOM | 3068 | SD | MET | B | 145 | −0.952 | 0.582 | 12.698 | 1.00 | 82.47 | S |
| ATOM | 3069 | CE | MET | B | 145 | −1.624 | 2.108 | 12.041 | 1.00 | 55.15 | C |
| ATOM | 3070 | N | THR | B | 146 | −5.673 | 1.744 | 11.601 | 1.00 | 43.69 | N |
| ATOM | 3071 | CA | THR | B | 146 | −6.393 | 1.643 | 10.339 | 1.00 | 39.91 | C |
| ATOM | 3072 | C | THR | B | 146 | −5.399 | 1.713 | 9.175 | 1.00 | 51.53 | C |
| ATOM | 3073 | O | THR | B | 146 | −4.188 | 1.705 | 9.396 | 1.00 | 56.89 | O |
| ATOM | 3074 | CB | THR | B | 146 | −7.464 | 2.751 | 10.198 | 1.00 | 55.94 | C |
| ATOM | 3075 | OG1 | THR | B | 146 | −6.845 | 4.045 | 10.230 | 1.00 | 52.14 | O |
| ATOM | 3076 | CG2 | THR | B | 146 | −8.494 | 2.646 | 11.322 | 1.00 | 51.55 | C |
| ATOM | 3077 | N | ARG | B | 147 | −5.894 | 1.775 | 7.941 | 1.00 | 53.99 | N |
| ATOM | 3078 | CA | ARG | B | 147 | −5.010 | 1.844 | 6.775 | 1.00 | 36.41 | C |
| ATOM | 3079 | C | ARG | B | 147 | −4.578 | 3.273 | 6.475 | 1.00 | 44.20 | C |
| ATOM | 3080 | O | ARG | B | 147 | −3.489 | 3.500 | 5.952 | 1.00 | 49.25 | O |
| ATOM | 3081 | CB | ARG | B | 147 | −5.680 | 1.256 | 5.532 | 1.00 | 54.91 | C |
| ATOM | 3082 | CG | ARG | B | 147 | −5.843 | −0.242 | 5.553 | 1.00 | 68.69 | C |
| ATOM | 3083 | CD | ARG | B | 147 | −6.363 | −0.734 | 4.221 | 1.00 | 73.99 | C |
| ATOM | 3084 | NE | ARG | B | 147 | −6.896 | −2.086 | 4.327 | 1.00 | 83.99 | N |
| ATOM | 3085 | CZ | ARG | B | 147 | −7.809 | −2.583 | 3.503 | 1.00 | 88.03 | C |
| ATOM | 3086 | NH1 | ARG | B | 147 | −8.287 | −1.829 | 2.520 | 1.00 | 91.81 | N |
| ATOM | 3087 | NH2 | ARG | B | 147 | −8.250 | −3.824 | 3.667 | 1.00 | 82.20 | N |
| ATOM | 3088 | N | ALA | B | 148 | −5.431 | 4.240 | 6.792 | 1.00 | 47.27 | N |
| ATOM | 3089 | CA | ALA | B | 148 | −5.082 | 5.631 | 6.558 | 1.00 | 46.62 | C |
| ATOM | 3090 | C | ALA | B | 148 | −3.859 | 5.994 | 7.387 | 1.00 | 40.08 | C |
| ATOM | 3091 | O | ALA | B | 148 | −3.060 | 6.847 | 6.994 | 1.00 | 43.11 | O |
| ATOM | 3092 | CB | ALA | B | 148 | −6.245 | 6.534 | 6.886 | 1.00 | 37.66 | C |
| ATOM | 3093 | N | ARG | B | 149 | −3.710 | 5.319 | 8.521 | 1.00 | 31.19 | N |
| ATOM | 3094 | CA | ARG | B | 149 | −2.613 | 5.589 | 9.436 | 1.00 | 47.60 | C |
| ATOM | 3095 | C | ARG | B | 149 | −1.299 | 4.927 | 9.047 | 1.00 | 43.94 | C |
| ATOM | 3096 | O | ARG | B | 149 | −0.229 | 5.515 | 9.218 | 1.00 | 34.83 | O |
| ATOM | 3097 | CB | ARG | B | 149 | −3.009 | 5.237 | 10.869 | 1.00 | 46.20 | C |
| ATOM | 3098 | CG | ARG | B | 149 | −3.860 | 6.319 | 11.489 | 1.00 | 49.51 | C |
| ATOM | 3099 | CD | ARG | B | 149 | −4.276 | 6.006 | 12.901 | 1.00 | 53.03 | C |
| ATOM | 3100 | NE | ARG | B | 149 | −5.336 | 6.919 | 13.295 | 1.00 | 57.25 | N |
| ATOM | 3101 | CZ | ARG | B | 149 | −6.246 | 6.650 | 14.217 | 1.00 | 59.07 | C |
| ATOM | 3102 | NH1 | ARG | B | 149 | −6.221 | 5.485 | 14.847 | 1.00 | 54.96 | N |
| ATOM | 3103 | NH2 | ARG | B | 149 | −7.181 | 7.546 | 14.502 | 1.00 | 71.12 | N |
| ATOM | 3104 | N | ALA | B | 150 | −1.382 | 3.709 | 8.529 | 1.00 | 36.22 | N |
| ATOM | 3105 | CA | ALA | B | 150 | −0.204 | 3.054 | 7.988 | 1.00 | 38.88 | C |
| ATOM | 3106 | C | ALA | B | 150 | 0.393 | 3.904 | 6.860 | 1.00 | 37.17 | C |
| ATOM | 3107 | O | ALA | B | 150 | 1.612 | 4.081 | 6.785 | 1.00 | 33.88 | O |
| ATOM | 3108 | CB | ALA | B | 150 | −0.544 | 1.656 | 7.497 | 1.00 | 41.50 | C |
| ATOM | 3109 | N | LYS | B | 151 | −0.460 | 4.437 | 5.990 | 1.00 | 30.74 | N |
| ATOM | 3110 | CA | LYS | B | 151 | 0.002 | 5.375 | 4.976 | 1.00 | 31.80 | C |
| ATOM | 3111 | C | LYS | B | 151 | 0.681 | 6.585 | 5.631 | 1.00 | 41.80 | C |
| ATOM | 3112 | O | LYS | B | 151 | 1.792 | 6.983 | 5.239 | 1.00 | 36.84 | O |
| ATOM | 3113 | CB | LYS | B | 151 | −1.142 | 5.842 | 4.086 | 1.00 | 25.15 | C |
| ATOM | 3114 | CG | LYS | B | 151 | −1.673 | 4.806 | 3.112 | 1.00 | 40.94 | C |
| ATOM | 3115 | CD | LYS | B | 151 | −2.760 | 5.433 | 2.236 | 1.00 | 58.78 | C |
| ATOM | 3116 | CE | LYS | B | 151 | −3.876 | 4.450 | 1.894 | 1.00 | 66.59 | C |
| ATOM | 3117 | NZ | LYS | B | 151 | −5.175 | 5.168 | 1.690 | 1.00 | 60.94 | N |
| ATOM | 3118 | N | VAL | B | 152 | 0.019 | 7.166 | 6.631 | 1.00 | 39.15 | N |
| ATOM | 3119 | CA | VAL | B | 152 | 0.611 | 8.276 | 7.373 | 1.00 | 36.37 | C |
| ATOM | 3120 | C | VAL | B | 152 | 1.944 | 7.860 | 8.003 | 1.00 | 31.40 | C |
| ATOM | 3121 | O | VAL | B | 152 | 2.902 | 8.624 | 7.988 | 1.00 | 27.52 | O |
| ATOM | 3122 | CB | VAL | B | 152 | −0.348 | 8.850 | 8.430 | 1.00 | 32.18 | C |
| ATOM | 3123 | CG1 | VAL | B | 152 | 0.413 | 9.715 | 9.418 | 1.00 | 39.20 | C |
| ATOM | 3124 | CG2 | VAL | B | 152 | −1.414 | 9.658 | 7.759 | 1.00 | 30.88 | C |
| ATOM | 3125 | N | ILE | B | 153 | 2.008 | 6.644 | 8.535 | 1.00 | 28.94 | N |
| ATOM | 3126 | CA | ILE | B | 153 | 3.262 | 6.131 | 9.071 | 1.00 | 30.79 | C |
| ATOM | 3127 | C | ILE | B | 153 | 4.334 | 5.984 | 7.987 | 1.00 | 28.84 | C |
| ATOM | 3128 | O | ILE | B | 153 | 5.462 | 6.444 | 8.162 | 1.00 | 21.84 | O |
| ATOM | 3129 | CB | ILE | B | 153 | 3.074 | 4.788 | 9.780 | 1.00 | 28.22 | C |
| ATOM | 3130 | CG1 | ILE | B | 153 | 2.172 | 4.971 | 10.995 | 1.00 | 35.39 | C |
| ATOM | 3131 | CG2 | ILE | B | 153 | 4.431 | 4.207 | 10.196 | 1.00 | 24.84 | C |
| ATOM | 3132 | CD1 | ILE | B | 153 | 1.869 | 3.692 | 11.736 | 1.00 | 30.64 | C |
| ATOM | 3133 | N | ILE | B | 154 | 3.982 | 5.340 | 6.875 | 1.00 | 26.13 | N |
| ATOM | 3134 | CA | ILE | B | 154 | 4.917 | 5.174 | 5.768 | 1.00 | 28.18 | C |
| ATOM | 3135 | C | ILE | B | 154 | 5.484 | 6.525 | 5.318 | 1.00 | 33.03 | C |
| ATOM | 3136 | O | ILE | B | 154 | 6.706 | 6.694 | 5.191 | 1.00 | 24.88 | O |
| ATOM | 3137 | CB | ILE | B | 154 | 4.267 | 4.459 | 4.579 | 1.00 | 27.18 | C |
| ATOM | 3138 | CG1 | ILE | B | 154 | 4.206 | 2.951 | 4.841 | 1.00 | 33.18 | C |
| ATOM | 3139 | CG2 | ILE | B | 154 | 5.053 | 4.740 | 3.307 | 1.00 | 24.58 | C |
| ATOM | 3140 | CD1 | ILE | B | 154 | 3.066 | 2.240 | 4.117 | 1.00 | 28.80 | C |
| ATOM | 3141 | N | CYS | B | 155 | 4.592 | 7.488 | 5.090 | 1.00 | 27.81 | N |
| ATOM | 3142 | CA | CYS | B | 155 | 5.010 | 8.841 | 4.743 | 1.00 | 24.89 | C |
| ATOM | 3143 | C | CYS | B | 155 | 5.993 | 9.461 | 5.746 | 1.00 | 31.35 | C |
| ATOM | 3144 | O | CYS | B | 155 | 6.936 | 10.150 | 5.364 | 1.00 | 28.69 | O |
| ATOM | 3145 | CB | CYS | B | 155 | 3.792 | 9.739 | 4.594 | 1.00 | 20.06 | C |
| ATOM | 3146 | SG | CYS | B | 155 | 2.939 | 9.455 | 3.069 | 1.00 | 41.60 | S |
| ATOM | 3147 | N | THR | B | 156 | 5.762 | 9.220 | 7.031 | 1.00 | 29.60 | N |
| ATOM | 3148 | CA | THR | B | 156 | 6.619 | 9.774 | 8.065 | 1.00 | 30.92 | C |
| ATOM | 3149 | C | THR | B | 156 | 8.015 | 9.133 | 8.037 | 1.00 | 25.63 | C |
| ATOM | 3150 | O | THR | B | 156 | 9.024 | 9.828 | 8.173 | 1.00 | 23.09 | O |
| ATOM | 3151 | CB | THR | B | 156 | 5.942 | 9.701 | 9.453 | 1.00 | 21.61 | C |
| ATOM | 3152 | OG1 | THR | B | 156 | 4.682 | 10.364 | 9.374 | 1.00 | 39.16 | O |
| ATOM | 3153 | CG2 | THR | B | 156 | 6.766 | 10.410 | 10.503 | 1.00 | 21.31 | C |
| ATOM | 3154 | N | VAL | B | 157 | 8.075 | 7.822 | 7.830 | 1.00 | 21.35 | N |
| ATOM | 3155 | CA | VAL | B | 157 | 9.361 | 7.163 | 7.616 | 1.00 | 30.10 | C |
| ATOM | 3156 | C | VAL | B | 157 | 10.140 | 7.752 | 6.410 | 1.00 | 28.51 | C |
| ATOM | 3157 | O | VAL | B | 157 | 11.345 | 7.984 | 6.495 | 1.00 | 26.93 | O |
| ATOM | 3158 | CB | VAL | B | 157 | 9.211 | 5.622 | 7.490 | 1.00 | 23.99 | C |
| ATOM | 3159 | CG1 | VAL | B | 157 | 10.514 | 4.982 | 7.015 | 1.00 | 21.87 | C |
| ATOM | 3160 | CG2 | VAL | B | 157 | 8.780 | 5.036 | 8.805 | 1.00 | 19.41 | C |
| ATOM | 3161 | N | TRP | B | 158 | 9.464 | 8.014 | 5.300 | 1.00 | 19.52 | N |
| ATOM | 3162 | CA | TRP | B | 158 | 10.166 | 8.599 | 4.168 | 1.00 | 21.62 | C |
| ATOM | 3163 | C | TRP | B | 158 | 10.610 | 10.043 | 4.446 | 1.00 | 21.29 | C |
| ATOM | 3164 | O | TRP | B | 158 | 11.593 | 10.502 | 3.881 | 1.00 | 20.92 | O |
| ATOM | 3165 | CB | TRP | B | 158 | 9.348 | 8.488 | 2.860 | 1.00 | 25.60 | C |
| ATOM | 3166 | CG | TRP | B | 158 | 9.391 | 7.111 | 2.223 | 1.00 | 21.80 | C |
| ATOM | 3167 | CD1 | TRP | B | 158 | 8.512 | 6.093 | 2.425 | 1.00 | 20.83 | C |
| ATOM | 3168 | CD2 | TRP | B | 158 | 10.375 | 6.610 | 1.306 | 1.00 | 28.29 | C |
| ATOM | 3169 | NE1 | TRP | B | 158 | 8.880 | 4.987 | 1.699 | 1.00 | 22.95 | N |
| ATOM | 3170 | CE2 | TRP | B | 158 | 10.020 | 5.273 | 1.004 | 1.00 | 25.48 | C |
| ATOM | 3171 | CE3 | TRP | B | 158 | 11.527 | 7.152 | 0.723 | 1.00 | 29.57 | C |
| ATOM | 3172 | CZ2 | TRP | B | 158 | 10.758 | 4.480 | 0.133 | 1.00 | 24.46 | C |
| ATOM | 3173 | CZ3 | TRP | B | 158 | 12.268 | 6.356 | −0.142 | 1.00 | 33.93 | C |
| ATOM | 3174 | CH2 | TRP | B | 158 | 11.875 | 5.035 | −0.430 | 1.00 | 34.66 | C |
| ATOM | 3175 | N | ALA | B | 159 | 9.905 | 10.755 | 5.322 | 1.00 | 20.11 | N |
| ATOM | 3176 | CA | ALA | B | 159 | 10.306 | 12.118 | 5.678 | 1.00 | 19.20 | C |
| ATOM | 3177 | C | ALA | B | 159 | 11.503 | 12.082 | 6.600 | 1.00 | 24.32 | C |
| ATOM | 3178 | O | ALA | B | 159 | 12.500 | 12.776 | 6.375 | 1.00 | 24.74 | O |
| ATOM | 3179 | CB | ALA | B | 159 | 9.172 | 12.877 | 6.341 | 1.00 | 18.08 | C |
| ATOM | 3180 | N | ILE | B | 160 | 11.399 | 11.272 | 7.649 | 1.00 | 23.41 | N |
| ATOM | 3181 | CA | ILE | B | 160 | 12.525 | 11.066 | 8.551 | 1.00 | 25.68 | C |
| ATOM | 3182 | C | ILE | B | 160 | 13.758 | 10.625 | 7.764 | 1.00 | 28.19 | C |
| ATOM | 3183 | O | ILE | B | 160 | 14.862 | 11.121 | 7.992 | 1.00 | 23.86 | O |
| ATOM | 3184 | CB | ILE | B | 160 | 12.190 | 10.046 | 9.653 | 1.00 | 22.20 | C |
| ATOM | 3185 | CG1 | ILE | B | 160 | 11.083 | 10.605 | 10.555 | 1.00 | 27.98 | C |
| ATOM | 3186 | CG2 | ILE | B | 160 | 13.440 | 9.704 | 10.470 | 1.00 | 20.50 | C |
| ATOM | 3187 | CD1 | ILE | B | 160 | 10.494 | 9.590 | 11.552 | 1.00 | 23.80 | C |
| ATOM | 3188 | N | SER | B | 161 | 13.555 | 9.708 | 6.818 | 1.00 | 27.90 | N |
| ATOM | 3189 | CA | SER | B | 161 | 14.656 | 9.215 | 6.002 | 1.00 | 28.39 | C |
| ATOM | 3190 | C | SER | B | 161 | 15.225 | 10.324 | 5.143 | 1.00 | 23.70 | C |
| ATOM | 3191 | O | SER | B | 161 | 16.435 | 10.451 | 5.052 | 1.00 | 22.04 | O |
| ATOM | 3192 | CB | SER | B | 161 | 14.241 | 8.024 | 5.143 | 1.00 | 31.58 | C |
| ATOM | 3193 | OG | SER | B | 161 | 14.013 | 6.875 | 5.936 | 1.00 | 32.14 | O |
| ATOM | 3194 | N | ALA | B | 162 | 14.357 | 11.125 | 4.525 | 1.00 | 22.36 | N |
| ATOM | 3195 | CA | ALA | B | 162 | 14.814 | 12.291 | 3.772 | 1.00 | 25.80 | C |
| ATOM | 3196 | C | ALA | B | 162 | 15.566 | 13.274 | 4.672 | 1.00 | 25.71 | C |
| ATOM | 3197 | O | ALA | B | 162 | 16.578 | 13.859 | 4.269 | 1.00 | 18.55 | O |
| ATOM | 3198 | CB | ALA | B | 162 | 13.649 | 12.983 | 3.089 | 1.00 | 11.29 | C |
| ATOM | 3199 | N | LEU | B | 163 | 15.064 | 13.450 | 5.892 | 1.00 | 20.35 | N |
| ATOM | 3200 | CA | LEU | B | 163 | 15.645 | 14.421 | 6.802 | 1.00 | 22.30 | C |
| ATOM | 3201 | C | LEU | B | 163 | 17.083 | 14.045 | 7.173 | 1.00 | 25.57 | C |
| ATOM | 3202 | O | LEU | B | 163 | 17.993 | 14.865 | 7.052 | 1.00 | 21.94 | O |
| ATOM | 3203 | CB | LEU | B | 163 | 14.792 | 14.576 | 8.058 | 1.00 | 24.55 | C |
| ATOM | 3204 | CG | LEU | B | 163 | 15.319 | 15.590 | 9.088 | 1.00 | 28.46 | C |
| ATOM | 3205 | CD1 | LEU | B | 163 | 15.348 | 17.008 | 8.520 | 1.00 | 25.41 | C |
| ATOM | 3206 | CD2 | LEU | B | 163 | 14.506 | 15.562 | 10.352 | 1.00 | 23.26 | C |
| ATOM | 3207 | N | VAL | B | 164 | 17.282 | 12.795 | 7.585 | 1.00 | 27.34 | N |
| ATOM | 3208 | CA | VAL | B | 164 | 18.567 | 12.352 | 8.129 | 1.00 | 28.31 | C |
| ATOM | 3209 | C | VAL | B | 164 | 19.617 | 11.907 | 7.117 | 1.00 | 31.69 | C |
| ATOM | 3210 | O | VAL | B | 164 | 20.775 | 11.698 | 7.487 | 1.00 | 43.15 | O |
| ATOM | 3211 | CB | VAL | B | 164 | 18.395 | 11.216 | 9.158 | 1.00 | 24.77 | C |
| ATOM | 3212 | CG1 | VAL | B | 164 | 17.432 | 11.648 | 10.237 | 1.00 | 26.60 | C |
| ATOM | 3213 | CG2 | VAL | B | 164 | 17.926 | 9.929 | 8.474 | 1.00 | 21.79 | C |
| ATOM | 3214 | N | SER | B | 165 | 19.237 | 11.763 | 5.852 | 1.00 | 28.74 | N |
| ATOM | 3215 | CA | SER | B | 165 | 20.192 | 11.262 | 4.859 | 1.00 | 32.18 | C |
| ATOM | 3216 | C | SER | B | 165 | 20.137 | 11.884 | 3.470 | 1.00 | 28.79 | C |
| ATOM | 3217 | O | SER | B | 165 | 21.110 | 11.823 | 2.732 | 1.00 | 42.76 | O |
| ATOM | 3218 | CB | SER | B | 165 | 20.151 | 9.730 | 4.766 | 1.00 | 41.86 | C |
| ATOM | 3219 | OG | SER | B | 165 | 18.848 | 9.214 | 4.939 | 1.00 | 47.47 | O |
| ATOM | 3220 | N | PHE | B | 166 | 19.024 | 12.487 | 3.100 | 1.00 | 30.85 | N |
| ATOM | 3221 | CA | PHE | B | 166 | 19.019 | 13.266 | 1.872 | 1.00 | 30.93 | C |
| ATOM | 3222 | C | PHE | B | 166 | 19.571 | 14.676 | 2.124 | 1.00 | 26.66 | C |
| ATOM | 3223 | O | PHE | B | 166 | 20.619 | 15.024 | 1.602 | 1.00 | 30.42 | O |
| ATOM | 3224 | CB | PHE | B | 166 | 17.614 | 13.325 | 1.284 | 1.00 | 29.94 | C |
| ATOM | 3225 | CG | PHE | B | 166 | 17.575 | 13.718 | −0.156 | 1.00 | 23.56 | C |
| ATOM | 3226 | CD1 | PHE | B | 166 | 17.395 | 15.041 | −0.522 | 1.00 | 22.01 | C |
| ATOM | 3227 | CD2 | PHE | B | 166 | 17.691 | 12.757 | −1.145 | 1.00 | 30.21 | C |
| ATOM | 3228 | CE1 | PHE | B | 166 | 17.346 | 15.400 | −1.846 | 1.00 | 24.58 | C |
| ATOM | 3229 | CE2 | PHE | B | 166 | 17.636 | 13.107 | −2.481 | 1.00 | 29.24 | C |
| ATOM | 3230 | CZ | PHE | B | 166 | 17.465 | 14.431 | −2.830 | 1.00 | 34.78 | C |
| ATOM | 3231 | N | LEU | B | 167 | 18.871 | 15.475 | 2.927 | 1.00 | 26.05 | N |
| ATOM | 3232 | CA | LEU | B | 167 | 19.321 | 16.834 | 3.253 | 1.00 | 36.33 | C |
| ATOM | 3233 | C | LEU | B | 167 | 20.830 | 17.031 | 3.531 | 1.00 | 35.42 | C |
| ATOM | 3234 | O | LEU | B | 167 | 21.449 | 17.897 | 2.915 | 1.00 | 34.94 | O |
| ATOM | 3235 | CB | LEU | B | 167 | 18.514 | 17.408 | 4.420 | 1.00 | 31.63 | C |
| ATOM | 3236 | CG | LEU | B | 167 | 17.093 | 17.857 | 4.111 | 1.00 | 43.74 | C |
| ATOM | 3237 | CD1 | LEU | B | 167 | 16.493 | 18.493 | 5.353 | 1.00 | 46.75 | C |
| ATOM | 3238 | CD2 | LEU | B | 167 | 17.087 | 18.840 | 2.953 | 1.00 | 38.63 | C |
| ATOM | 3239 | N | PRO | B | 168 | 21.413 | 16.253 | 4.473 | 1.00 | 31.97 | N |
| ATOM | 3240 | CA | PRO | B | 168 | 22.809 | 16.482 | 4.863 | 1.00 | 31.19 | C |
| ATOM | 3241 | C | PRO | B | 168 | 23.799 | 16.350 | 3.706 | 1.00 | 28.87 | C |
| ATOM | 3242 | O | PRO | B | 168 | 24.742 | 17.146 | 3.595 | 1.00 | 24.60 | O |
| ATOM | 3243 | CB | PRO | B | 168 | 23.061 | 15.381 | 5.899 | 1.00 | 25.80 | C |
| ATOM | 3244 | CG | PRO | B | 168 | 21.730 | 15.036 | 6.414 | 1.00 | 21.17 | C |
| ATOM | 3245 | CD | PRO | B | 168 | 20.814 | 15.156 | 5.252 | 1.00 | 27.03 | C |
| ATOM | 3246 | N | ILE | B | 169 | 23.587 | 15.344 | 2.864 | 1.00 | 27.07 | N |
| ATOM | 3247 | CA | ILE | B | 169 | 24.393 | 15.159 | 1.660 | 1.00 | 31.64 | C |
| ATOM | 3248 | C | ILE | B | 169 | 24.237 | 16.323 | 0.689 | 1.00 | 29.97 | C |
| ATOM | 3249 | O | ILE | B | 169 | 25.215 | 16.823 | 0.135 | 1.00 | 36.78 | O |
| ATOM | 3250 | CB | ILE | B | 169 | 24.049 | 13.838 | 0.958 | 1.00 | 23.25 | C |
| ATOM | 3251 | CG1 | ILE | B | 169 | 24.722 | 12.687 | 1.710 | 1.00 | 22.01 | C |
| ATOM | 3252 | CG2 | ILE | B | 169 | 24.504 | 13.881 | −0.483 | 1.00 | 19.29 | C |
| ATOM | 3253 | CD1 | ILE | B | 169 | 24.291 | 11.317 | 1.320 | 1.00 | 17.36 | C |
| ATOM | 3254 | N | MET | B | 170 | 23.001 | 16.759 | 0.501 | 1.00 | 23.63 | N |
| ATOM | 3255 | CA | MET | B | 170 | 22.724 | 17.931 | −0.312 | 1.00 | 29.18 | C |
| ATOM | 3256 | C | MET | B | 170 | 23.114 | 19.255 | 0.364 | 1.00 | 37.27 | C |
| ATOM | 3257 | O | MET | B | 170 | 23.206 | 20.283 | −0.295 | 1.00 | 37.41 | O |
| ATOM | 3258 | CB | MET | B | 170 | 21.253 | 17.940 | −0.724 | 1.00 | 39.60 | C |
| ATOM | 3259 | CG | MET | B | 170 | 20.835 | 16.715 | −1.566 | 1.00 | 48.63 | C |
| ATOM | 3260 | SD | MET | B | 170 | 21.160 | 16.861 | −3.347 | 1.00 | 52.26 | S |
| ATOM | 3261 | CE | MET | B | 170 | 22.931 | 16.602 | −3.395 | 1.00 | 32.83 | C |
| ATOM | 3262 | N | MET | B | 171 | 23.347 | 19.228 | 1.673 | 1.00 | 43.31 | N |
| ATOM | 3263 | CA | MET | B | 171 | 23.838 | 20.405 | 2.397 | 1.00 | 41.88 | C |
| ATOM | 3264 | C | MET | B | 171 | 25.369 | 20.418 | 2.467 | 1.00 | 37.87 | C |
| ATOM | 3265 | O | MET | B | 171 | 25.975 | 21.376 | 2.940 | 1.00 | 28.19 | O |
| ATOM | 3266 | CB | MET | B | 171 | 23.251 | 20.465 | 3.810 | 1.00 | 36.23 | C |
| ATOM | 3267 | CG | MET | B | 171 | 21.826 | 21.028 | 3.910 | 1.00 | 44.09 | C |
| ATOM | 3268 | SD | MET | B | 171 | 21.194 | 20.978 | 5.633 | 1.00 | 60.33 | S |
| ATOM | 3269 | CE | MET | B | 171 | 21.954 | 22.444 | 6.320 | 1.00 | 36.27 | C |
| ATOM | 3270 | N | HIS | B | 172 | 25.978 | 19.332 | 2.008 | 1.00 | 37.02 | N |
| ATOM | 3271 | CA | HIS | B | 172 | 27.433 | 19.239 | 1.858 | 1.00 | 41.42 | C |
| ATOM | 3272 | C | HIS | B | 172 | 28.193 | 18.988 | 3.142 | 1.00 | 26.86 | C |
| ATOM | 3273 | O | HIS | B | 172 | 29.387 | 19.254 | 3.202 | 1.00 | 32.33 | O |
| ATOM | 3274 | CB | HIS | B | 172 | 28.014 | 20.484 | 1.182 | 1.00 | 40.43 | C |
| ATOM | 3275 | CG | HIS | B | 172 | 27.321 | 20.855 | −0.090 | 1.00 | 50.66 | C |
| ATOM | 3276 | ND1 | HIS | B | 172 | 27.412 | 20.092 | −1.232 | 1.00 | 48.00 | N |
| ATOM | 3277 | CD2 | HIS | B | 172 | 26.524 | 21.907 | −0.393 | 1.00 | 47.54 | C |
| ATOM | 3278 | CE1 | HIS | B | 172 | 26.700 | 20.662 | −2.190 | 1.00 | 57.15 | C |
| ATOM | 3279 | NE2 | HIS | B | 172 | 26.150 | 21.761 | −1.709 | 1.00 | 52.67 | N |
| ATOM | 3280 | N | TRP | B | 173 | 27.518 | 18.468 | 4.155 | 1.00 | 27.67 | N |
| ATOM | 3281 | CA | TRP | B | 173 | 28.156 | 18.239 | 5.445 | 1.00 | 27.24 | C |
| ATOM | 3282 | C | TRP | B | 173 | 29.097 | 17.032 | 5.406 | 1.00 | 34.23 | C |
| ATOM | 3283 | O | TRP | B | 173 | 29.865 | 16.796 | 6.338 | 1.00 | 31.68 | O |
| ATOM | 3284 | CB | TRP | B | 173 | 27.094 | 17.996 | 6.511 | 1.00 | 32.78 | C |
| ATOM | 3285 | CG | TRP | B | 173 | 26.234 | 19.176 | 6.852 | 1.00 | 29.18 | C |
| ATOM | 3286 | CD1 | TRP | B | 173 | 26.241 | 20.407 | 6.267 | 1.00 | 31.97 | C |
| ATOM | 3287 | CD2 | TRP | B | 173 | 25.206 | 19.209 | 7.842 | 1.00 | 23.43 | C |
| ATOM | 3288 | NE1 | TRP | B | 173 | 25.285 | 21.208 | 6.840 | 1.00 | 26.94 | N |
| ATOM | 3289 | CE2 | TRP | B | 173 | 24.637 | 20.492 | 7.811 | 1.00 | 31.29 | C |
| ATOM | 3290 | CE3 | TRP | B | 173 | 24.709 | 18.272 | 8.749 | 1.00 | 24.38 | C |
| ATOM | 3291 | CZ2 | TRP | B | 173 | 23.602 | 20.864 | 8.663 | 1.00 | 34.07 | C |
| ATOM | 3292 | CZ3 | TRP | B | 173 | 23.677 | 18.640 | 9.585 | 1.00 | 21.75 | C |
| ATOM | 3293 | CH2 | TRP | B | 173 | 23.142 | 19.925 | 9.542 | 1.00 | 25.79 | C |
| ATOM | 3294 | N | TRP | B | 174 | 29.021 | 16.263 | 4.329 | 1.00 | 27.35 | N |
| ATOM | 3295 | CA | TRP | B | 174 | 29.763 | 15.018 | 4.222 | 1.00 | 25.08 | C |
| ATOM | 3296 | C | TRP | B | 174 | 31.203 | 15.255 | 3.759 | 1.00 | 29.43 | C |
| ATOM | 3297 | O | TRP | B | 174 | 32.015 | 14.328 | 3.743 | 1.00 | 27.85 | O |
| ATOM | 3298 | CB | TRP | B | 174 | 29.051 | 14.071 | 3.242 | 1.00 | 29.47 | C |
| ATOM | 3299 | CG | TRP | B | 174 | 28.915 | 14.659 | 1.846 | 1.00 | 32.12 | C |
| ATOM | 3300 | CD1 | TRP | B | 174 | 27.998 | 15.582 | 1.431 | 1.00 | 29.68 | C |
| ATOM | 3301 | CD2 | TRP | B | 174 | 29.735 | 14.375 | 0.706 | 1.00 | 26.16 | C |
| ATOM | 3302 | NE1 | TRP | B | 174 | 28.197 | 15.890 | 0.113 | 1.00 | 30.04 | N |
| ATOM | 3303 | CE2 | TRP | B | 174 | 29.259 | 15.164 | −0.357 | 1.00 | 33.12 | C |
| ATOM | 3304 | CE3 | TRP | B | 174 | 30.827 | 13.533 | 0.484 | 1.00 | 29.71 | C |
| ATOM | 3305 | CZ2 | TRP | B | 174 | 29.830 | 15.130 | −1.629 | 1.00 | 34.84 | C |
| ATOM | 3306 | CZ3 | TRP | B | 174 | 31.392 | 13.502 | −0.774 | 1.00 | 38.57 | C |
| ATOM | 3307 | CH2 | TRP | B | 174 | 30.894 | 14.298 | −1.816 | 1.00 | 31.02 | C |
| ATOM | 3308 | N | ARG | B | 175 | 31.532 | 16.489 | 3.387 | 1.00 | 23.54 | N |
| ATOM | 3309 | CA | ARG | B | 175 | 32.822 | 16.730 | 2.733 | 1.00 | 33.88 | C |
| ATOM | 3310 | C | ARG | B | 175 | 34.022 | 16.825 | 3.669 | 1.00 | 33.51 | C |
| ATOM | 3311 | O | ARG | B | 175 | 33.929 | 17.346 | 4.782 | 1.00 | 31.00 | O |
| ATOM | 3312 | CB | ARG | B | 175 | 32.778 | 17.939 | 1.794 | 1.00 | 26.16 | C |
| ATOM | 3313 | CG | ARG | B | 175 | 31.690 | 17.845 | 0.733 | 1.00 | 33.20 | C |
| ATOM | 3314 | CD | ARG | B | 175 | 32.147 | 18.393 | −0.601 | 1.00 | 32.36 | C |
| ATOM | 3315 | NE | ARG | B | 175 | 31.266 | 19.446 | −1.091 | 1.00 | 44.66 | N |
| ATOM | 3316 | CZ | ARG | B | 175 | 30.894 | 19.585 | −2.361 | 1.00 | 62.45 | C |
| ATOM | 3317 | NH1 | ARG | B | 175 | 31.302 | 18.716 | −3.275 | 1.00 | 55.21 | N |
| ATOM | 3318 | NH2 | ARG | B | 175 | 30.089 | 20.580 | −2.716 | 1.00 | 66.54 | N |
| ATOM | 3319 | N | ASP | B | 176 | 35.148 | 16.314 | 3.182 | 1.00 | 29.66 | N |
| ATOM | 3320 | CA | ASP | B | 176 | 36.412 | 16.346 | 3.894 | 1.00 | 29.09 | C |
| ATOM | 3321 | C | ASP | B | 176 | 37.175 | 17.605 | 3.523 | 1.00 | 28.50 | C |
| ATOM | 3322 | O | ASP | B | 176 | 36.738 | 18.364 | 2.677 | 1.00 | 31.32 | O |
| ATOM | 3323 | CB | ASP | B | 176 | 37.234 | 15.110 | 3.546 | 1.00 | 41.23 | C |
| ATOM | 3324 | CG | ASP | B | 176 | 38.171 | 14.704 | 4.662 | 1.00 | 53.28 | C |
| ATOM | 3325 | OD1 | ASP | B | 176 | 38.533 | 15.579 | 5.477 | 1.00 | 48.28 | O |
| ATOM | 3326 | OD2 | ASP | B | 176 | 38.540 | 13.506 | 4.722 | 1.00 | 66.48 | O |
| ATOM | 3327 | N | GLU | B | 177 | 38.316 | 17.823 | 4.161 | 1.00 | 41.01 | N |
| ATOM | 3328 | CA | GLU | B | 177 | 39.061 | 19.066 | 3.995 | 1.00 | 44.93 | C |
| ATOM | 3329 | C | GLU | B | 177 | 40.357 | 18.786 | 3.248 | 1.00 | 50.53 | C |
| ATOM | 3330 | O | GLU | B | 177 | 40.924 | 19.664 | 2.584 | 1.00 | 31.93 | O |
| ATOM | 3331 | CB | GLU | B | 177 | 39.347 | 19.680 | 5.364 | 1.00 | 63.15 | C |
| ATOM | 3332 | CG | GLU | B | 177 | 39.442 | 21.182 | 5.321 | 1.00 | 86.75 | C |
| ATOM | 3333 | CD | GLU | B | 177 | 38.583 | 21.762 | 4.213 | 1.00 | 85.78 | C |
| ATOM | 3334 | OE1 | GLU | B | 177 | 37.389 | 21.402 | 4.145 | 1.00 | 85.47 | O |
| ATOM | 3335 | OE2 | GLU | B | 177 | 39.102 | 22.572 | 3.409 | 1.00 | 88.09 | O |
| ATOM | 3336 | N | ASP | B | 178 | 40.782 | 17.530 | 3.366 | 1.00 | 46.54 | N |
| ATOM | 3337 | CA | ASP | B | 178 | 41.948 | 16.950 | 2.699 | 1.00 | 45.18 | C |
| ATOM | 3338 | C | ASP | B | 178 | 42.112 | 17.330 | 1.232 | 1.00 | 46.32 | C |
| ATOM | 3339 | O | ASP | B | 178 | 41.133 | 17.395 | 0.491 | 1.00 | 54.70 | O |
| ATOM | 3340 | CB | ASP | B | 178 | 41.847 | 15.423 | 2.789 | 1.00 | 48.19 | C |
| ATOM | 3341 | CG | ASP | B | 178 | 43.134 | 14.725 | 2.403 | 1.00 | 65.42 | C |
| ATOM | 3342 | OD1 | ASP | B | 178 | 44.223 | 15.252 | 2.718 | 1.00 | 69.89 | O |
| ATOM | 3343 | OD2 | ASP | B | 178 | 43.061 | 13.635 | 1.797 | 1.00 | 73.91 | O |
| ATOM | 3344 | N | PRO | B | 179 | 43.363 | 17.573 | 0.809 | 1.00 | 52.81 | N |
| ATOM | 3345 | CA | PRO | B | 179 | 43.759 | 17.720 | −0.595 | 1.00 | 50.10 | C |
| ATOM | 3346 | C | PRO | B | 179 | 43.084 | 16.700 | −1.525 | 1.00 | 47.27 | C |
| ATOM | 3347 | O | PRO | B | 179 | 42.458 | 17.089 | −2.520 | 1.00 | 33.85 | O |
| ATOM | 3348 | CB | PRO | B | 179 | 45.267 | 17.456 | −0.545 | 1.00 | 48.43 | C |
| ATOM | 3349 | CG | PRO | B | 179 | 45.678 | 17.968 | 0.777 | 1.00 | 45.63 | C |
| ATOM | 3350 | CD | PRO | B | 179 | 44.500 | 17.807 | 1.717 | 1.00 | 51.57 | C |
| ATOM | 3351 | N | GLN | B | 180 | 43.221 | 15.413 | −1.213 | 1.00 | 39.18 | N |
| ATOM | 3352 | CA | GLN | B | 180 | 42.651 | 14.372 | −2.059 | 1.00 | 42.14 | C |
| ATOM | 3353 | C | GLN | B | 180 | 41.139 | 14.507 | −2.214 | 1.00 | 42.79 | C |
| ATOM | 3354 | O | GLN | B | 180 | 40.602 | 14.340 | −3.311 | 1.00 | 37.64 | O |
| ATOM | 3355 | CB | GLN | B | 180 | 43.011 | 12.987 | −1.534 | 1.00 | 49.57 | C |
| ATOM | 3356 | CG | GLN | B | 180 | 44.455 | 12.603 | −1.798 | 1.00 | 78.53 | C |
| ATOM | 3357 | CD | GLN | B | 180 | 44.756 | 11.162 | −1.421 | 1.00 | 110.67 | C |
| ATOM | 3358 | OE1 | GLN | B | 180 | 43.849 | 10.330 | −1.319 | 1.00 | 115.43 | O |
| ATOM | 3359 | NE2 | GLN | B | 180 | 46.036 | 10.858 | −1.215 | 1.00 | 116.46 | N |
| ATOM | 3360 | N | ALA | B | 181 | 40.459 | 14.810 | −1.115 | 1.00 | 35.88 | N |
| ATOM | 3361 | CA | ALA | B | 181 | 39.025 | 15.033 | −1.153 | 1.00 | 33.81 | C |
| ATOM | 3362 | C | ALA | B | 181 | 38.712 | 16.187 | −2.097 | 1.00 | 36.68 | C |
| ATOM | 3363 | O | ALA | B | 181 | 37.875 | 16.055 | −2.994 | 1.00 | 33.02 | O |
| ATOM | 3364 | CB | ALA | B | 181 | 38.482 | 15.313 | 0.243 | 1.00 | 34.53 | C |
| ATOM | 3365 | N | LEU | B | 182 | 39.388 | 17.314 | −1.907 | 1.00 | 34.70 | N |
| ATOM | 3366 | CA | LEU | B | 182 | 39.147 | 18.485 | −2.753 | 1.00 | 43.10 | C |
| ATOM | 3367 | C | LEU | B | 182 | 39.403 | 18.189 | −4.231 | 1.00 | 39.58 | C |
| ATOM | 3368 | O | LEU | B | 182 | 38.674 | 18.661 | −5.104 | 1.00 | 39.79 | O |
| ATOM | 3369 | CB | LEU | B | 182 | 39.991 | 19.678 | −2.292 | 1.00 | 49.61 | C |
| ATOM | 3370 | CG | LEU | B | 182 | 39.541 | 20.315 | −0.976 | 1.00 | 57.54 | C |
| ATOM | 3371 | CD1 | LEU | B | 182 | 40.292 | 21.610 | −0.738 | 1.00 | 56.18 | C |
| ATOM | 3372 | CD2 | LEU | B | 182 | 38.030 | 20.549 | −0.981 | 1.00 | 38.78 | C |
| ATOM | 3373 | N | LYS | B | 183 | 40.441 | 17.403 | −4.502 | 1.00 | 38.11 | N |
| ATOM | 3374 | CA | LYS | B | 183 | 40.723 | 16.939 | −5.853 | 1.00 | 36.86 | C |
| ATOM | 3375 | C | LYS | B | 183 | 39.517 | 16.229 | −6.441 | 1.00 | 35.27 | C |
| ATOM | 3376 | O | LYS | B | 183 | 39.167 | 16.442 | −7.592 | 1.00 | 33.65 | O |
| ATOM | 3377 | CB | LYS | B | 183 | 41.912 | 15.986 | −5.843 | 1.00 | 44.97 | C |
| ATOM | 3378 | CG | LYS | B | 183 | 42.466 | 15.689 | −7.207 | 1.00 | 39.35 | C |
| ATOM | 3379 | CD | LYS | B | 183 | 43.814 | 15.019 | −7.104 | 1.00 | 48.35 | C |
| ATOM | 3380 | CE | LYS | B | 183 | 44.369 | 14.720 | −8.486 | 1.00 | 63.07 | C |
| ATOM | 3381 | NZ | LYS | B | 183 | 45.478 | 13.727 | −8.423 | 1.00 | 84.28 | N |
| ATOM | 3382 | N | CYS | B | 184 | 38.887 | 15.376 | −5.641 | 1.00 | 36.46 | N |
| ATOM | 3383 | CA | CYS | B | 184 | 37.739 | 14.609 | −6.098 | 1.00 | 33.84 | C |
| ATOM | 3384 | C | CYS | B | 184 | 36.537 | 15.503 | −6.384 | 1.00 | 36.61 | C |
| ATOM | 3385 | O | CYS | B | 184 | 35.858 | 15.349 | −7.411 | 1.00 | 33.79 | O |
| ATOM | 3386 | CB | CYS | B | 184 | 37.362 | 13.541 | −5.070 | 1.00 | 33.65 | C |
| ATOM | 3387 | SG | CYS | B | 184 | 35.999 | 12.453 | −5.605 | 1.00 | 64.51 | S |
| ATOM | 3388 | N | TYR | B | 185 | 36.273 | 16.434 | −5.472 | 1.00 | 31.25 | N |
| ATOM | 3389 | CA | TYR | B | 185 | 35.098 | 17.288 | −5.599 | 1.00 | 34.98 | C |
| ATOM | 3390 | C | TYR | B | 185 | 35.121 | 18.132 | −6.876 | 1.00 | 36.81 | C |
| ATOM | 3391 | O | TYR | B | 185 | 34.086 | 18.605 | −7.341 | 1.00 | 36.62 | O |
| ATOM | 3392 | CB | TYR | B | 185 | 34.926 | 18.181 | −4.375 | 1.00 | 37.06 | C |
| ATOM | 3393 | CG | TYR | B | 185 | 34.876 | 17.417 | −3.078 | 1.00 | 37.46 | C |
| ATOM | 3394 | CD1 | TYR | B | 185 | 34.389 | 16.115 | −3.038 | 1.00 | 24.84 | C |
| ATOM | 3395 | CD2 | TYR | B | 185 | 35.316 | 18.003 | −1.883 | 1.00 | 28.18 | C |
| ATOM | 3396 | CE1 | TYR | B | 185 | 34.358 | 15.408 | −1.840 | 1.00 | 36.09 | C |
| ATOM | 3397 | CE2 | TYR | B | 185 | 35.289 | 17.308 | −0.689 | 1.00 | 25.25 | C |
| ATOM | 3398 | CZ | TYR | B | 185 | 34.808 | 16.015 | −0.664 | 1.00 | 29.55 | C |
| ATOM | 3399 | OH | TYR | B | 185 | 34.765 | 15.324 | 0.527 | 1.00 | 26.04 | O |
| ATOM | 3400 | N | GLN | B | 186 | 36.297 | 18.302 | −7.457 | 1.00 | 38.65 | N |
| ATOM | 3401 | CA | GLN | B | 186 | 36.406 | 19.127 | −8.649 | 1.00 | 47.36 | C |
| ATOM | 3402 | C | GLN | B | 186 | 36.527 | 18.336 | −9.947 | 1.00 | 37.15 | C |
| ATOM | 3403 | O | GLN | B | 186 | 36.475 | 18.900 | −11.039 | 1.00 | 42.73 | O |
| ATOM | 3404 | CB | GLN | B | 186 | 37.551 | 20.121 | −8.506 | 1.00 | 45.51 | C |
| ATOM | 3405 | CG | GLN | B | 186 | 37.144 | 21.346 | −7.734 | 1.00 | 45.88 | C |
| ATOM | 3406 | CD | GLN | B | 186 | 37.889 | 22.551 | −8.200 | 1.00 | 70.74 | C |
| ATOM | 3407 | OE1 | GLN | B | 186 | 38.516 | 22.528 | −9.260 | 1.00 | 93.61 | O |
| ATOM | 3408 | NE2 | GLN | B | 186 | 37.840 | 23.618 | −7.415 | 1.00 | 94.50 | N |
| ATOM | 3409 | N | ASP | B | 187 | 36.683 | 17.030 | −9.823 | 1.00 | 28.21 | N |
| ATOM | 3410 | CA | ASP | B | 187 | 36.701 | 16.176 | −10.986 | 1.00 | 34.18 | C |
| ATOM | 3411 | C | ASP | B | 187 | 35.291 | 15.633 | −11.190 | 1.00 | 33.43 | C |
| ATOM | 3412 | O | ASP | B | 187 | 34.823 | 14.834 | −10.374 | 1.00 | 39.57 | O |
| ATOM | 3413 | CB | ASP | B | 187 | 37.708 | 15.043 | −10.781 | 1.00 | 33.95 | C |
| ATOM | 3414 | CG | ASP | B | 187 | 37.787 | 14.090 | −11.974 | 1.00 | 48.87 | C |
| ATOM | 3415 | OD1 | ASP | B | 187 | 37.295 | 14.435 | −13.078 | 1.00 | 42.99 | O |
| ATOM | 3416 | OD2 | ASP | B | 187 | 38.349 | 12.983 | −11.798 | 1.00 | 57.70 | O |
| ATOM | 3417 | N | PRO | B | 188 | 34.597 | 16.091 | −12.258 | 1.00 | 24.88 | N |
| ATOM | 3418 | CA | PRO | B | 188 | 33.258 | 15.602 | −12.601 | 1.00 | 24.83 | C |
| ATOM | 3419 | C | PRO | B | 188 | 33.264 | 14.112 | −12.864 | 1.00 | 26.40 | C |
| ATOM | 3420 | O | PRO | B | 188 | 32.226 | 13.460 | −12.787 | 1.00 | 34.03 | O |
| ATOM | 3421 | CB | PRO | B | 188 | 32.919 | 16.369 | −13.876 | 1.00 | 19.85 | C |
| ATOM | 3422 | CG | PRO | B | 188 | 33.685 | 17.610 | −13.784 | 1.00 | 19.23 | C |
| ATOM | 3423 | CD | PRO | B | 188 | 34.971 | 17.251 | −13.083 | 1.00 | 28.92 | C |
| ATOM | 3424 | N | GLY | B | 189 | 34.438 | 13.576 | −13.157 | 1.00 | 26.01 | N |
| ATOM | 3425 | CA | GLY | B | 189 | 34.590 | 12.148 | −13.329 | 1.00 | 29.28 | C |
| ATOM | 3426 | C | GLY | B | 189 | 34.657 | 11.380 | −12.020 | 1.00 | 25.49 | C |
| ATOM | 3427 | O | GLY | B | 189 | 34.288 | 10.210 | −11.968 | 1.00 | 34.11 | O |
| ATOM | 3428 | N | CYS | B | 190 | 35.142 | 12.014 | −10.960 | 1.00 | 24.21 | N |
| ATOM | 3429 | CA | CYS | B | 190 | 35.222 | 11.332 | −9.675 | 1.00 | 38.21 | C |
| ATOM | 3430 | C | CYS | B | 190 | 33.900 | 11.396 | −8.903 | 1.00 | 39.26 | C |
| ATOM | 3431 | O | CYS | B | 190 | 33.380 | 12.489 | −8.630 | 1.00 | 38.91 | O |
| ATOM | 3432 | CB | CYS | B | 190 | 36.375 | 11.880 | −8.835 | 1.00 | 30.13 | C |
| ATOM | 3433 | SG | CYS | B | 190 | 36.492 | 11.184 | −7.155 | 1.00 | 56.40 | S |
| ATOM | 3434 | N | CYS | B | 191 | 33.354 | 10.218 | −8.585 | 1.00 | 31.65 | N |
| ATOM | 3435 | CA | CYS | B | 191 | 32.152 | 10.113 | −7.752 | 1.00 | 38.91 | C |
| ATOM | 3436 | C | CYS | B | 191 | 32.392 | 9.225 | −6.532 | 1.00 | 39.92 | C |
| ATOM | 3437 | O | CYS | B | 191 | 31.601 | 8.329 | −6.246 | 1.00 | 47.76 | O |
| ATOM | 3438 | CB | CYS | B | 191 | 30.939 | 9.601 | −8.549 | 1.00 | 27.11 | C |
| ATOM | 3439 | SG | CYS | B | 191 | 29.312 | 10.105 | −7.836 | 1.00 | 35.39 | S |
| ATOM | 3440 | N | ASP | B | 192 | 33.490 | 9.473 | −5.823 | 1.00 | 46.50 | N |
| ATOM | 3441 | CA | ASP | B | 192 | 33.793 | 8.744 | −4.599 | 1.00 | 40.11 | C |
| ATOM | 3442 | C | ASP | B | 192 | 33.089 | 9.422 | −3.462 | 1.00 | 39.66 | C |
| ATOM | 3443 | O | ASP | B | 192 | 33.142 | 10.645 | −3.329 | 1.00 | 45.89 | O |
| ATOM | 3444 | CB | ASP | B | 192 | 35.285 | 8.764 | −4.289 | 1.00 | 38.06 | C |
| ATOM | 3445 | CG | ASP | B | 192 | 36.094 | 7.968 | −5.267 | 1.00 | 56.95 | C |
| ATOM | 3446 | OD1 | ASP | B | 192 | 35.639 | 7.798 | −6.417 | 1.00 | 65.13 | O |
| ATOM | 3447 | OD2 | ASP | B | 192 | 37.200 | 7.527 | −4.887 | 1.00 | 76.07 | O |
| ATOM | 3448 | N | PHE | B | 193 | 32.433 | 8.632 | −2.629 | 1.00 | 39.96 | N |
| ATOM | 3449 | CA | PHE | B | 193 | 31.824 | 9.190 | −1.436 | 1.00 | 40.43 | C |
| ATOM | 3450 | C | PHE | B | 193 | 32.914 | 9.351 | −0.365 | 1.00 | 35.27 | C |
| ATOM | 3451 | O | PHE | B | 193 | 32.964 | 8.626 | 0.637 | 1.00 | 36.87 | O |
| ATOM | 3452 | CB | PHE | B | 193 | 30.643 | 8.330 | −0.972 | 1.00 | 27.56 | C |
| ATOM | 3453 | CG | PHE | B | 193 | 29.669 | 9.068 | −0.113 | 1.00 | 33.24 | C |
| ATOM | 3454 | CD1 | PHE | B | 193 | 29.432 | 10.429 | −0.325 | 1.00 | 23.81 | C |
| ATOM | 3455 | CD2 | PHE | B | 193 | 28.981 | 8.405 | 0.904 | 1.00 | 25.58 | C |
| ATOM | 3456 | CE1 | PHE | B | 193 | 28.529 | 11.118 | 0.458 | 1.00 | 21.82 | C |
| ATOM | 3457 | CE2 | PHE | B | 193 | 28.087 | 9.081 | 1.690 | 1.00 | 27.28 | C |
| ATOM | 3458 | CZ | PHE | B | 193 | 27.855 | 10.449 | 1.470 | 1.00 | 27.50 | C |
| ATOM | 3459 | N | VAL | B | 194 | 33.802 | 10.307 | −0.620 | 1.00 | 30.43 | N |
| ATOM | 3460 | CA | VAL | B | 194 | 34.897 | 10.646 | 0.279 | 1.00 | 31.33 | C |
| ATOM | 3461 | C | VAL | B | 194 | 34.396 | 11.602 | 1.341 | 1.00 | 30.10 | C |
| ATOM | 3462 | O | VAL | B | 194 | 34.169 | 12.782 | 1.067 | 1.00 | 29.14 | O |
| ATOM | 3463 | CB | VAL | B | 194 | 36.036 | 11.329 | −0.503 | 1.00 | 31.58 | C |
| ATOM | 3464 | CG1 | VAL | B | 194 | 37.131 | 11.823 | 0.448 | 1.00 | 28.55 | C |
| ATOM | 3465 | CG2 | VAL | B | 194 | 36.587 | 10.378 | −1.548 | 1.00 | 21.23 | C |
| ATOM | 3466 | N | THR | B | 195 | 34.222 | 11.103 | 2.559 | 1.00 | 32.69 | N |
| ATOM | 3467 | CA | THR | B | 195 | 33.554 | 11.896 | 3.596 | 1.00 | 33.28 | C |
| ATOM | 3468 | C | THR | B | 195 | 34.462 | 12.196 | 4.767 | 1.00 | 30.39 | C |
| ATOM | 3469 | O | THR | B | 195 | 35.440 | 11.481 | 5.000 | 1.00 | 44.74 | O |
| ATOM | 3470 | CB | THR | B | 195 | 32.264 | 11.211 | 4.104 | 1.00 | 30.84 | C |
| ATOM | 3471 | OG1 | THR | B | 195 | 32.582 | 9.943 | 4.689 | 1.00 | 28.03 | O |
| ATOM | 3472 | CG2 | THR | B | 195 | 31.288 | 10.991 | 2.949 | 1.00 | 31.49 | C |
| ATOM | 3473 | N | ASN | B | 196 | 34.148 | 13.259 | 5.499 | 1.00 | 28.76 | N |
| ATOM | 3474 | CA | ASN | B | 196 | 34.856 | 13.516 | 6.745 | 1.00 | 32.00 | C |
| ATOM | 3475 | C | ASN | B | 196 | 34.459 | 12.465 | 7.791 | 1.00 | 26.85 | C |
| ATOM | 3476 | O | ASN | B | 196 | 33.366 | 11.891 | 7.721 | 1.00 | 26.63 | O |
| ATOM | 3477 | CB | ASN | B | 196 | 34.622 | 14.950 | 7.228 | 1.00 | 28.61 | C |
| ATOM | 3478 | CG | ASN | B | 196 | 33.188 | 15.202 | 7.654 | 1.00 | 28.63 | C |
| ATOM | 3479 | OD1 | ASN | B | 196 | 32.667 | 14.544 | 8.547 | 1.00 | 29.84 | O |
| ATOM | 3480 | ND2 | ASN | B | 196 | 32.557 | 16.180 | 7.035 | 1.00 | 26.35 | N |
| ATOM | 3481 | N | ARG | B | 197 | 35.349 | 12.196 | 8.742 | 1.00 | 31.48 | N |
| ATOM | 3482 | CA | ARG | B | 197 | 35.124 | 11.105 | 9.696 | 1.00 | 38.59 | C |
| ATOM | 3483 | C | ARG | B | 197 | 33.928 | 11.323 | 10.636 | 1.00 | 29.75 | C |
| ATOM | 3484 | O | ARG | B | 197 | 33.282 | 10.362 | 11.058 | 1.00 | 24.11 | O |
| ATOM | 3485 | CB | ARG | B | 197 | 36.397 | 10.787 | 10.486 | 1.00 | 26.34 | C |
| ATOM | 3486 | CG | ARG | B | 197 | 37.520 | 10.258 | 9.622 | 1.00 | 46.41 | C |
| ATOM | 3487 | CD | ARG | B | 197 | 38.661 | 9.703 | 10.448 | 1.00 | 48.65 | C |
| ATOM | 3488 | NE | ARG | B | 197 | 39.796 | 9.309 | 9.614 | 1.00 | 67.46 | N |
| ATOM | 3489 | CZ | ARG | B | 197 | 40.915 | 8.754 | 10.080 | 1.00 | 89.84 | C |
| ATOM | 3490 | NH1 | ARG | B | 197 | 41.063 | 8.518 | 11.381 | 1.00 | 96.11 | N |
| ATOM | 3491 | NH2 | ARG | B | 197 | 41.891 | 8.431 | 9.245 | 1.00 | 91.14 | N |
| ATOM | 3492 | N | ALA | B | 198 | 33.630 | 12.582 | 10.947 | 1.00 | 19.28 | N |
| ATOM | 3493 | CA | ALA | B | 198 | 32.480 | 12.879 | 11.780 | 1.00 | 16.86 | C |
| ATOM | 3494 | C | ALA | B | 198 | 31.246 | 12.347 | 11.089 | 1.00 | 29.06 | C |
| ATOM | 3495 | O | ALA | B | 198 | 30.514 | 11.537 | 11.652 | 1.00 | 27.43 | O |
| ATOM | 3496 | CB | ALA | B | 198 | 32.353 | 14.364 | 12.015 | 1.00 | 22.57 | C |
| ATOM | 3497 | N | TYR | B | 199 | 31.044 | 12.781 | 9.845 | 1.00 | 32.96 | N |
| ATOM | 3498 | CA | TYR | B | 199 | 29.864 | 12.411 | 9.083 | 1.00 | 20.39 | C |
| ATOM | 3499 | C | TYR | B | 199 | 29.802 | 10.912 | 8.824 | 1.00 | 23.78 | C |
| ATOM | 3500 | O | TYR | B | 199 | 28.720 | 10.327 | 8.806 | 1.00 | 27.29 | O |
| ATOM | 3501 | CB | TYR | B | 199 | 29.780 | 13.198 | 7.777 | 1.00 | 26.17 | C |
| ATOM | 3502 | CG | TYR | B | 199 | 28.713 | 12.662 | 6.858 | 1.00 | 27.90 | C |
| ATOM | 3503 | CD1 | TYR | B | 199 | 27.491 | 13.307 | 6.719 | 1.00 | 29.01 | C |
| ATOM | 3504 | CD2 | TYR | B | 199 | 28.917 | 11.480 | 6.156 | 1.00 | 25.29 | C |
| ATOM | 3505 | CE1 | TYR | B | 199 | 26.512 | 12.796 | 5.884 | 1.00 | 28.40 | C |
| ATOM | 3506 | CE2 | TYR | B | 199 | 27.958 | 10.963 | 5.340 | 1.00 | 27.93 | C |
| ATOM | 3507 | CZ | TYR | B | 199 | 26.760 | 11.619 | 5.200 | 1.00 | 27.76 | C |
| ATOM | 3508 | OH | TYR | B | 199 | 25.824 | 11.073 | 4.368 | 1.00 | 31.71 | O |
| ATOM | 3509 | N | ALA | B | 200 | 30.953 | 10.280 | 8.625 | 1.00 | 26.87 | N |
| ATOM | 3510 | CA | ALA | B | 200 | 30.963 | 8.828 | 8.423 | 1.00 | 36.15 | C |
| ATOM | 3511 | C | ALA | B | 200 | 30.335 | 8.084 | 9.612 | 1.00 | 30.08 | C |
| ATOM | 3512 | O | ALA | B | 200 | 29.521 | 7.189 | 9.433 | 1.00 | 26.22 | O |
| ATOM | 3513 | CB | ALA | B | 200 | 32.369 | 8.330 | 8.167 | 1.00 | 21.71 | C |
| ATOM | 3514 | N | ILE | B | 201 | 30.718 | 8.462 | 10.824 | 1.00 | 25.42 | N |
| ATOM | 3515 | CA | ILE | B | 201 | 30.202 | 7.805 | 12.015 | 1.00 | 32.00 | C |
| ATOM | 3516 | C | ILE | B | 201 | 28.750 | 8.191 | 12.338 | 1.00 | 28.76 | C |
| ATOM | 3517 | O | ILE | B | 201 | 27.889 | 7.332 | 12.487 | 1.00 | 23.84 | O |
| ATOM | 3518 | CB | ILE | B | 201 | 31.102 | 8.081 | 13.224 | 1.00 | 31.60 | C |
| ATOM | 3519 | CG1 | ILE | B | 201 | 32.412 | 7.291 | 13.087 | 1.00 | 35.75 | C |
| ATOM | 3520 | CG2 | ILE | B | 201 | 30.396 | 7.691 | 14.495 | 1.00 | 26.78 | C |
| ATOM | 3521 | CD1 | ILE | B | 201 | 33.583 | 7.930 | 13.805 | 1.00 | 24.95 | C |
| ATOM | 3522 | N | ALA | B | 202 | 28.484 | 9.487 | 12.428 | 1.00 | 23.11 | N |
| ATOM | 3523 | CA | ALA | B | 202 | 27.152 | 9.958 | 12.740 | 1.00 | 22.93 | C |
| ATOM | 3524 | C | ALA | B | 202 | 26.112 | 9.281 | 11.861 | 1.00 | 28.21 | C |
| ATOM | 3525 | O | ALA | B | 202 | 25.120 | 8.746 | 12.362 | 1.00 | 29.74 | O |
| ATOM | 3526 | CB | ALA | B | 202 | 27.066 | 11.480 | 12.612 | 1.00 | 19.08 | C |
| ATOM | 3527 | N | SER | B | 203 | 26.356 | 9.281 | 10.556 | 1.00 | 26.21 | N |
| ATOM | 3528 | CA | SER | B | 203 | 25.358 | 8.832 | 9.586 | 1.00 | 28.72 | C |
| ATOM | 3529 | C | SER | B | 203 | 25.188 | 7.315 | 9.473 | 1.00 | 25.18 | C |
| ATOM | 3530 | O | SER | B | 203 | 24.104 | 6.851 | 9.151 | 1.00 | 23.12 | O |
| ATOM | 3531 | CB | SER | B | 203 | 25.616 | 9.438 | 8.203 | 1.00 | 25.52 | C |
| ATOM | 3532 | OG | SER | B | 203 | 26.663 | 8.754 | 7.551 | 1.00 | 32.74 | O |
| ATOM | 3533 | N | SER | B | 204 | 26.226 | 6.527 | 9.728 | 1.00 | 23.72 | N |
| ATOM | 3534 | CA | SER | B | 204 | 25.993 | 5.078 | 9.745 | 1.00 | 34.76 | C |
| ATOM | 3535 | C | SER | B | 204 | 25.224 | 4.656 | 10.996 | 1.00 | 32.22 | C |
| ATOM | 3536 | O | SER | B | 204 | 24.301 | 3.839 | 10.926 | 1.00 | 30.64 | O |
| ATOM | 3537 | CB | SER | B | 204 | 27.261 | 4.230 | 9.513 | 1.00 | 27.27 | C |
| ATOM | 3538 | OG | SER | B | 204 | 28.438 | 4.989 | 9.623 | 1.00 | 37.04 | O |
| ATOM | 3539 | N | ILE | B | 205 | 25.586 | 5.237 | 12.131 | 1.00 | 27.63 | N |
| ATOM | 3540 | CA | ILE | B | 205 | 24.847 | 5.002 | 13.357 | 1.00 | 28.28 | C |
| ATOM | 3541 | C | ILE | B | 205 | 23.377 | 5.384 | 13.208 | 1.00 | 34.25 | C |
| ATOM | 3542 | O | ILE | B | 205 | 22.481 | 4.615 | 13.565 | 1.00 | 37.83 | O |
| ATOM | 3543 | CB | ILE | B | 205 | 25.422 | 5.814 | 14.500 | 1.00 | 22.60 | C |
| ATOM | 3544 | CG1 | ILE | B | 205 | 26.782 | 5.248 | 14.894 | 1.00 | 27.28 | C |
| ATOM | 3545 | CG2 | ILE | B | 205 | 24.448 | 5.810 | 15.683 | 1.00 | 19.72 | C |
| ATOM | 3546 | CD1 | ILE | B | 205 | 27.603 | 6.209 | 15.749 | 1.00 | 29.17 | C |
| ATOM | 3547 | N | ILE | B | 206 | 23.140 | 6.577 | 12.672 | 1.00 | 29.75 | N |
| ATOM | 3548 | CA | ILE | B | 206 | 21.801 | 7.161 | 12.594 | 1.00 | 22.22 | C |
| ATOM | 3549 | C | ILE | B | 206 | 20.927 | 6.563 | 11.496 | 1.00 | 28.02 | C |
| ATOM | 3550 | O | ILE | B | 206 | 19.722 | 6.431 | 11.659 | 1.00 | 31.41 | O |
| ATOM | 3551 | CB | ILE | B | 206 | 21.899 | 8.680 | 12.372 | 1.00 | 20.83 | C |
| ATOM | 3552 | CG1 | ILE | B | 206 | 22.323 | 9.363 | 13.666 | 1.00 | 33.25 | C |
| ATOM | 3553 | CG2 | ILE | B | 206 | 20.597 | 9.246 | 11.881 | 1.00 | 24.46 | C |
| ATOM | 3554 | CD1 | ILE | B | 206 | 22.514 | 10.843 | 13.528 | 1.00 | 40.23 | C |
| ATOM | 3555 | N | SER | B | 207 | 21.539 | 6.212 | 10.368 | 1.00 | 37.02 | N |
| ATOM | 3556 | CA | SER | B | 207 | 20.803 | 5.696 | 9.220 | 1.00 | 26.33 | C |
| ATOM | 3557 | C | SER | B | 207 | 20.688 | 4.174 | 9.251 | 1.00 | 31.72 | C |
| ATOM | 3558 | O | SER | B | 207 | 19.790 | 3.604 | 8.627 | 1.00 | 30.82 | O |
| ATOM | 3559 | CB | SER | B | 207 | 21.459 | 6.146 | 7.903 | 1.00 | 27.46 | C |
| ATOM | 3560 | OG | SER | B | 207 | 21.490 | 7.566 | 7.762 | 1.00 | 36.00 | O |
| ATOM | 3561 | N | PHE | B | 208 | 21.587 | 3.519 | 9.983 | 1.00 | 28.52 | N |
| ATOM | 3562 | CA | PHE | B | 208 | 21.653 | 2.063 | 9.979 | 1.00 | 27.99 | C |
| ATOM | 3563 | C | PHE | B | 208 | 21.723 | 1.403 | 11.372 | 1.00 | 28.51 | C |
| ATOM | 3564 | O | PHE | B | 208 | 20.868 | 0.591 | 11.702 | 1.00 | 30.41 | O |
| ATOM | 3565 | CB | PHE | B | 208 | 22.812 | 1.604 | 9.075 | 1.00 | 29.07 | C |
| ATOM | 3566 | CG | PHE | B | 208 | 22.892 | 0.110 | 8.881 | 1.00 | 31.13 | C |
| ATOM | 3567 | CD1 | PHE | B | 208 | 22.111 | −0.527 | 7.927 | 1.00 | 26.29 | C |
| ATOM | 3568 | CD2 | PHE | B | 208 | 23.760 | −0.657 | 9.651 | 1.00 | 33.70 | C |
| ATOM | 3569 | CE1 | PHE | B | 208 | 22.194 | −1.898 | 7.749 | 1.00 | 29.05 | C |
| ATOM | 3570 | CE2 | PHE | B | 208 | 23.845 | −2.035 | 9.482 | 1.00 | 30.79 | C |
| ATOM | 3571 | CZ | PHE | B | 208 | 23.063 | −2.654 | 8.531 | 1.00 | 33.65 | C |
| ATOM | 3572 | N | TYR | B | 209 | 22.721 | 1.743 | 12.186 | 1.00 | 31.12 | N |
| ATOM | 3573 | CA | TYR | B | 209 | 22.931 | 1.030 | 13.460 | 1.00 | 35.65 | C |
| ATOM | 3574 | C | TYR | B | 209 | 21.767 | 1.069 | 14.433 | 1.00 | 33.44 | C |
| ATOM | 3575 | O | TYR | B | 209 | 21.255 | 0.026 | 14.826 | 1.00 | 36.21 | O |
| ATOM | 3576 | CB | TYR | B | 209 | 24.240 | 1.447 | 14.136 | 1.00 | 31.39 | C |
| ATOM | 3577 | CG | TYR | B | 209 | 25.390 | 0.833 | 13.397 | 1.00 | 43.70 | C |
| ATOM | 3578 | CD1 | TYR | B | 209 | 26.186 | 1.595 | 12.548 | 1.00 | 35.09 | C |
| ATOM | 3579 | CD2 | TYR | B | 209 | 25.623 | −0.535 | 13.474 | 1.00 | 35.22 | C |
| ATOM | 3580 | CE1 | TYR | B | 209 | 27.210 | 1.023 | 11.832 | 1.00 | 30.85 | C |
| ATOM | 3581 | CE2 | TYR | B | 209 | 26.649 | −1.117 | 12.763 | 1.00 | 42.15 | C |
| ATOM | 3582 | CZ | TYR | B | 209 | 27.439 | −0.337 | 11.943 | 1.00 | 38.40 | C |
| ATOM | 3583 | OH | TYR | B | 209 | 28.460 | −0.934 | 11.245 | 1.00 | 35.90 | O |
| ATOM | 3584 | N | ILE | B | 210 | 21.353 | 2.269 | 14.812 | 1.00 | 31.77 | N |
| ATOM | 3585 | CA | ILE | B | 210 | 20.180 | 2.439 | 15.660 | 1.00 | 35.13 | C |
| ATOM | 3586 | C | ILE | B | 210 | 18.948 | 1.694 | 15.147 | 1.00 | 34.58 | C |
| ATOM | 3587 | O | ILE | B | 210 | 18.384 | 0.863 | 15.866 | 1.00 | 31.66 | O |
| ATOM | 3588 | CB | ILE | B | 210 | 19.833 | 3.919 | 15.830 | 1.00 | 29.49 | C |
| ATOM | 3589 | CG1 | ILE | B | 210 | 20.835 | 4.575 | 16.777 | 1.00 | 24.87 | C |
| ATOM | 3590 | CG2 | ILE | B | 210 | 18.397 | 4.073 | 16.319 | 1.00 | 30.97 | C |
| ATOM | 3591 | CD1 | ILE | B | 210 | 20.784 | 6.078 | 16.729 | 1.00 | 34.98 | C |
| ATOM | 3592 | N | PRO | B | 211 | 18.522 | 1.989 | 13.904 | 1.00 | 34.83 | N |
| ATOM | 3593 | CA | PRO | B | 211 | 17.340 | 1.291 | 13.387 | 1.00 | 32.09 | C |
| ATOM | 3594 | C | PRO | B | 211 | 17.563 | −0.208 | 13.345 | 1.00 | 32.70 | C |
| ATOM | 3595 | O | PRO | B | 211 | 16.602 | −0.968 | 13.402 | 1.00 | 36.41 | O |
| ATOM | 3596 | CB | PRO | B | 211 | 17.208 | 1.826 | 11.969 | 1.00 | 23.58 | C |
| ATOM | 3597 | CG | PRO | B | 211 | 17.926 | 3.133 | 11.983 | 1.00 | 27.75 | C |
| ATOM | 3598 | CD | PRO | B | 211 | 19.056 | 2.961 | 12.935 | 1.00 | 28.27 | C |
| ATOM | 3599 | N | LEU | B | 212 | 18.816 | −0.634 | 13.265 | 1.00 | 27.34 | N |
| ATOM | 3600 | CA | LEU | B | 212 | 19.093 | −2.055 | 13.159 | 1.00 | 31.54 | C |
| ATOM | 3601 | C | LEU | B | 212 | 18.958 | −2.725 | 14.509 | 1.00 | 39.16 | C |
| ATOM | 3602 | O | LEU | B | 212 | 18.290 | −3.748 | 14.652 | 1.00 | 36.88 | O |
| ATOM | 3603 | CB | LEU | B | 212 | 20.489 | −2.304 | 12.618 | 1.00 | 32.85 | C |
| ATOM | 3604 | CG | LEU | B | 212 | 20.680 | −3.810 | 12.539 | 1.00 | 33.38 | C |
| ATOM | 3605 | CD1 | LEU | B | 212 | 20.072 | −4.295 | 11.251 | 1.00 | 32.29 | C |
| ATOM | 3606 | CD2 | LEU | B | 212 | 22.140 | −4.201 | 12.660 | 1.00 | 33.74 | C |
| ATOM | 3607 | N | LEU | B | 213 | 19.617 | −2.147 | 15.502 | 1.00 | 37.23 | N |
| ATOM | 3608 | CA | LEU | B | 213 | 19.480 | −2.626 | 16.861 | 1.00 | 32.79 | C |
| ATOM | 3609 | C | LEU | B | 213 | 18.006 | −2.744 | 17.211 | 1.00 | 36.53 | C |
| ATOM | 3610 | O | LEU | B | 213 | 17.531 | −3.843 | 17.472 | 1.00 | 3.7.95 | O |
| ATOM | 3611 | CB | LEU | B | 213 | 20.226 | −1.707 | 17.816 | 1.00 | 31.98 | C |
| ATOM | 3612 | CG | LEU | B | 213 | 21.694 | −1.670 | 17.383 | 1.00 | 43.51 | C |
| ATOM | 3613 | CD1 | LEU | B | 213 | 22.561 | −0.762 | 18.252 | 1.00 | 38.20 | C |
| ATOM | 3614 | CD2 | LEU | B | 213 | 22.246 | −3.093 | 17.356 | 1.00 | 37.60 | C |
| ATOM | 3615 | N | ILE | B | 214 | 17.277 | −1.629 | 17.172 | 1.00 | 35.82 | N |
| ATOM | 3616 | CA | ILE | B | 214 | 15.849 | −1.642 | 17.487 | 1.00 | 34.50 | C |
| ATOM | 3617 | C | ILE | B | 214 | 15.142 | −2.795 | 16.782 | 1.00 | 40.13 | C |
| ATOM | 3618 | O | ILE | B | 214 | 14.441 | −3.581 | 17.411 | 1.00 | 42.45 | O |
| ATOM | 3619 | CB | ILE | B | 214 | 15.153 | −0.325 | 17.106 | 1.00 | 31.60 | C |
| ATOM | 3620 | CG1 | ILE | B | 214 | 15.521 | 0.780 | 18.087 | 1.00 | 24.13 | C |
| ATOM | 3621 | CG2 | ILE | B | 214 | 13.632 | −0.491 | 17.108 | 1.00 | 26.92 | C |
| ATOM | 3622 | CD1 | ILE | B | 214 | 15.146 | 2.158 | 17.574 | 1.00 | 23.20 | C |
| ATOM | 3623 | N | MET | B | 215 | 15.332 | −2.907 | 15.477 | 1.00 | 33.41 | N |
| ATOM | 3624 | CA | MET | B | 215 | 14.655 | −3.960 | 14.744 | 1.00 | 39.32 | C |
| ATOM | 3625 | C | MET | B | 215 | 15.044 | −5.343 | 15.232 | 1.00 | 42.70 | C |
| ATOM | 3626 | O | MET | B | 215 | 14.211 | −6.239 | 15.282 | 1.00 | 44.83 | O |
| ATOM | 3627 | CB | MET | B | 215 | 14.941 | −3.887 | 13.251 | 1.00 | 47.17 | C |
| ATOM | 3628 | CG | MET | B | 215 | 14.268 | −5.023 | 12.504 | 1.00 | 50.64 | C |
| ATOM | 3629 | SD | MET | B | 215 | 14.968 | −5.323 | 10.887 | 1.00 | 55.93 | S |
| ATOM | 3630 | CE | MET | B | 215 | 16.609 | −5.867 | 11.337 | 1.00 | 38.75 | C |
| ATOM | 3631 | N | ILE | B | 216 | 16.315 | −5.522 | 15.567 | 1.00 | 47.13 | N |
| ATOM | 3632 | CA | ILE | B | 216 | 16.805 | −6.828 | 15.992 | 1.00 | 45.31 | C |
| ATOM | 3633 | C | ILE | B | 216 | 16.153 | −7.232 | 17.311 | 1.00 | 47.08 | C |
| ATOM | 3634 | O | ILE | B | 216 | 15.614 | −8.331 | 17.431 | 1.00 | 44.99 | O |
| ATOM | 3635 | CB | ILE | B | 216 | 18.352 | −6.851 | 16.090 | 1.00 | 54.97 | C |
| ATOM | 3636 | CG1 | ILE | B | 216 | 18.963 | −7.205 | 14.725 | 1.00 | 54.55 | C |
| ATOM | 3637 | CG2 | ILE | B | 216 | 18.822 | −7.838 | 17.157 | 1.00 | 45.21 | C |
| ATOM | 3638 | CD1 | ILE | B | 216 | 20.450 | −6.878 | 14.588 | 1.00 | 43.41 | C |
| ATOM | 3639 | N | PHE | B | 217 | 16.197 | −6.320 | 18.278 | 1.00 | 47.45 | N |
| ATOM | 3640 | CA | PHE | B | 217 | 15.571 | −6.484 | 19.588 | 1.00 | 44.77 | C |
| ATOM | 3641 | C | PHE | B | 217 | 14.080 | −6.792 | 19.451 | 1.00 | 48.75 | C |
| ATOM | 3642 | O | PHE | B | 217 | 13.635 | −7.915 | 19.704 | 1.00 | 49.23 | O |
| ATOM | 3643 | CB | PHE | B | 217 | 15.794 | −5.198 | 20.398 | 1.00 | 47.14 | C |
| ATOM | 3644 | CG | PHE | B | 217 | 15.130 | −5.180 | 21.757 | 1.00 | 73.92 | C |
| ATOM | 3645 | CD1 | PHE | B | 217 | 15.868 | −5.428 | 22.911 | 1.00 | 72.76 | C |
| ATOM | 3646 | CD2 | PHE | B | 217 | 13.779 | −4.861 | 21.889 | 1.00 | 67.29 | C |
| ATOM | 3647 | CE1 | PHE | B | 217 | 15.261 | −5.392 | 24.167 | 1.00 | 74.63 | C |
| ATOM | 3648 | CE2 | PHE | B | 217 | 13.169 | −4.823 | 23.142 | 1.00 | 57.41 | C |
| ATOM | 3649 | CZ | PHE | B | 217 | 13.909 | −5.089 | 24.279 | 1.00 | 63.47 | C |
| ATOM | 3650 | N | VAL | B | 218 | 13.316 | −5.790 | 19.038 | 1.00 | 47.45 | N |
| ATOM | 3651 | CA | VAL | B | 218 | 11.873 | −5.923 | 18.901 | 1.00 | 38.01 | C |
| ATOM | 3652 | C | VAL | B | 218 | 11.535 | −7.227 | 18.187 | 1.00 | 38.38 | C |
| ATOM | 3653 | O | VAL | B | 218 | 10.567 | −7.899 | 18.530 | 1.00 | 46.50 | O |
| ATOM | 3654 | CB | VAL | B | 218 | 11.273 | −4.693 | 18.166 | 1.00 | 35.28 | C |
| ATOM | 3655 | CG1 | VAL | B | 218 | 9.811 | −4.910 | 17.803 | 1.00 | 30.61 | C |
| ATOM | 3656 | CG2 | VAL | B | 218 | 11.449 | −3.435 | 19.019 | 1.00 | 27.70 | C |
| ATOM | 3657 | N | ALA | B | 219 | 12.355 | −7.598 | 17.215 | 1.00 | 36.89 | N |
| ATOM | 3658 | CA | ALA | B | 219 | 12.123 | −8.812 | 16.444 | 1.00 | 42.43 | C |
| ATOM | 3659 | C | ALA | B | 219 | 12.294 | −10.069 | 17.286 | 1.00 | 51.60 | C |
| ATOM | 3660 | O | ALA | B | 219 | 11.493 | −10.996 | 17.190 | 1.00 | 54.50 | O |
| ATOM | 3661 | CB | ALA | B | 219 | 13.043 | −8.862 | 15.241 | 1.00 | 41.27 | C |
| ATOM | 3662 | N | LEU | B | 220 | 13.344 | −10.111 | 18.097 | 1.00 | 47.34 | N |
| ATOM | 3663 | CA | LEU | B | 220 | 13.565 | −11.251 | 18.973 | 1.00 | 48.94 | C |
| ATOM | 3664 | C | LEU | B | 220 | 12.377 | −11.423 | 19.907 | 1.00 | 58.20 | C |
| ATOM | 3665 | O | LEU | B | 220 | 11.912 | −12.539 | 20.136 | 1.00 | 57.79 | O |
| ATOM | 3666 | CB | LEU | B | 220 | 14.860 | −11.085 | 19.767 | 1.00 | 46.53 | C |
| ATOM | 3667 | CG | LEU | B | 220 | 16.119 | −11.234 | 18.907 | 1.00 | 58.64 | C |
| ATOM | 3668 | CD1 | LEU | B | 220 | 17.363 | −10.833 | 19.678 | 1.00 | 47.59 | C |
| ATOM | 3669 | CD2 | LEU | B | 220 | 16.242 | −12.656 | 18.357 | 1.00 | 43.62 | C |
| ATOM | 3670 | N | ARG | B | 221 | 11.875 | −10.308 | 20.425 | 1.00 | 44.87 | N |
| ATOM | 3671 | CA | ARG | B | 221 | 10.718 | −10.338 | 21.305 | 1.00 | 49.56 | C |
| ATOM | 3672 | C | ARG | B | 221 | 9.537 | −11.037 | 20.641 | 1.00 | 60.06 | C |
| ATOM | 3673 | O | ARG | B | 221 | 8.938 | −11.950 | 21.212 | 1.00 | 62.85 | O |
| ATOM | 3674 | CB | ARG | B | 221 | 10.326 | −8.920 | 21.725 | 1.00 | 58.87 | C |
| ATOM | 3675 | CG | ARG | B | 221 | 11.290 | −8.275 | 22.711 | 1.00 | 59.77 | C |
| ATOM | 3676 | CD | ARG | B | 221 | 11.163 | −8.895 | 24.090 | 1.00 | 84.85 | C |
| ATOM | 3677 | NE | ARG | B | 221 | 12.215 | −8.449 | 25.000 | 1.00 | 109.64 | N |
| ATOM | 3678 | CZ | ARG | B | 221 | 12.251 | −8.740 | 26.299 | 1.00 | 128.22 | C |
| ATOM | 3679 | NH1 | ARG | B | 221 | 11.288 | −9.476 | 26.842 | 1.00 | 127.61 | N |
| ATOM | 3680 | NH2 | ARG | B | 221 | 13.247 | −8.296 | 27.058 | 1.00 | 119.15 | N |
| ATOM | 3681 | N | VAL | B | 222 | 9.201 | −10.605 | 19.432 | 1.00 | 61.50 | N |
| ATOM | 3682 | CA | VAL | B | 222 | 8.106 | −11.214 | 18.692 | 1.00 | 53.88 | C |
| ATOM | 3683 | C | VAL | B | 222 | 8.268 | −12.729 | 18.587 | 1.00 | 58.23 | C |
| ATOM | 3684 | O | VAL | B | 222 | 7.281 | −13.464 | 18.599 | 1.00 | 56.04 | O |
| ATOM | 3685 | CB | VAL | B | 222 | 7.986 | −10.609 | 17.295 | 1.00 | 43.18 | C |
| ATOM | 3686 | CG1 | VAL | B | 222 | 6.887 | −11.311 | 16.499 | 1.00 | 45.51 | C |
| ATOM | 3687 | CG2 | VAL | B | 222 | 7.713 | −9.120 | 17.405 | 1.00 | 43.31 | C |
| ATOM | 3688 | N | TYR | B | 223 | 9.512 | −13.194 | 18.499 | 1.00 | 62.09 | N |
| ATOM | 3689 | CA | TYR | B | 223 | 9.773 | −14.628 | 18.402 | 1.00 | 69.09 | C |
| ATOM | 3690 | C | TYR | B | 223 | 9.394 | −15.348 | 19.687 | 1.00 | 65.03 | C |
| ATOM | 3691 | O | TYR | B | 223 | 8.723 | −16.374 | 19.657 | 1.00 | 66.56 | O |
| ATOM | 3692 | CB | TYR | B | 223 | 11.239 | −14.903 | 18.079 | 1.00 | 65.47 | C |
| ATOM | 3693 | CG | TYR | B | 223 | 11.513 | −16.356 | 17.760 | 1.00 | 69.06 | C |
| ATOM | 3694 | CD1 | TYR | B | 223 | 10.976 | −16.944 | 16.626 | 1.00 | 74.10 | C |
| ATOM | 3695 | CD2 | TYR | B | 223 | 12.308 | −17.139 | 18.589 | 1.00 | 76.99 | C |
| ATOM | 3696 | CE1 | TYR | B | 223 | 11.221 | −18.271 | 16.322 | 1.00 | 88.31 | C |
| ATOM | 3697 | CE2 | TYR | B | 223 | 12.561 | −18.469 | 18.292 | 1.00 | 83.75 | C |
| ATOM | 3698 | CZ | TYR | B | 223 | 12.014 | −19.028 | 17.159 | 1.00 | 86.98 | C |
| ATOM | 3699 | OH | TYR | B | 223 | 12.259 | −20.348 | 16.860 | 1.00 | 94.51 | O |
| ATOM | 3700 | N | ARG | B | 224 | 9.841 | −14.810 | 20.815 | 1.00 | 63.26 | N |
| ATOM | 3701 | CA | ARG | B | 224 | 9.526 | −15.406 | 22.105 | 1.00 | 70.75 | C |
| ATOM | 3702 | C | ARG | B | 224 | 8.023 | −15.430 | 22.299 | 1.00 | 73.98 | C |
| ATOM | 3703 | O | ARG | B | 224 | 7.470 | −16.417 | 22.778 | 1.00 | 77.48 | O |
| ATOM | 3704 | CB | ARG | B | 224 | 10.224 | −14.651 | 23.240 | 1.00 | 60.44 | C |
| ATOM | 3705 | CG | ARG | B | 224 | 11.721 | −14.928 | 23.294 | 1.00 | 76.25 | C |
| ATOM | 3706 | CD | ARG | B | 224 | 12.499 | −13.882 | 24.077 | 1.00 | 86.71 | C |
| ATOM | 3707 | NE | ARG | B | 224 | 13.928 | −13.972 | 23.775 | 1.00 | 101.98 | N |
| ATOM | 3708 | CZ | ARG | B | 224 | 14.889 | −13.372 | 24.472 | 1.00 | 108.15 | C |
| ATOM | 3709 | NH1 | ARG | B | 224 | 14.584 | −12.632 | 25.528 | 1.00 | 107.44 | N |
| ATOM | 3710 | NH2 | ARG | B | 224 | 16.161 | −13.518 | 24.116 | 1.00 | 95.70 | N |
| ATOM | 3711 | N | GLU | B | 225 | 7.368 | −14.348 | 21.893 | 1.00 | 70.71 | N |
| ATOM | 3712 | CA | GLU | B | 225 | 5.921 | −14.239 | 22.009 | 1.00 | 70.98 | C |
| ATOM | 3713 | C | GLU | B | 225 | 5.187 | −15.268 | 21.171 | 1.00 | 72.14 | C |
| ATOM | 3714 | O | GLU | B | 225 | 4.176 | −15.816 | 21.599 | 1.00 | 85.72 | O |
| ATOM | 3715 | CB | GLU | B | 225 | 5.457 | −12.836 | 21.629 | 1.00 | 71.19 | C |
| ATOM | 3716 | CG | GLU | B | 225 | 5.481 | −11.877 | 22.793 | 1.00 | 86.76 | C |
| ATOM | 3717 | CD | GLU | B | 225 | 4.663 | −12.387 | 23.965 | 1.00 | 107.93 | C |
| ATOM | 3718 | OE1 | GLU | B | 225 | 3.746 | −13.210 | 23.735 | 1.00 | 107.13 | O |
| ATOM | 3719 | OE2 | GLU | B | 225 | 4.938 | −11.966 | 25.110 | 1.00 | 103.05 | O |
| ATOM | 3720 | N | ALA | B | 226 | 5.688 | −15.524 | 19.971 | 1.00 | 74.32 | N |
| ATOM | 3721 | CA | ALA | B | 226 | 5.055 | −16.495 | 19.095 | 1.00 | 81.63 | C |
| ATOM | 3722 | C | ALA | B | 226 | 5.204 | −17.896 | 19.685 | 1.00 | 89.58 | C |
| ATOM | 3723 | O | ALA | B | 226 | 4.307 | −18.730 | 19.552 | 1.00 | 94.07 | O |
| ATOM | 3724 | CB | ALA | B | 226 | 5.648 | −16.421 | 17.698 | 1.00 | 72.68 | C |
| ATOM | 3725 | N | LYS | B | 227 | 6.335 | −18.138 | 20.346 | 1.00 | 85.80 | N |
| ATOM | 3726 | CA | LYS | B | 227 | 6.598 | −19.411 | 21.014 | 1.00 | 78.60 | C |
| ATOM | 3727 | C | LYS | B | 227 | 5.703 | −19.595 | 22.240 | 1.00 | 86.72 | C |
| ATOM | 3728 | O | LYS | B | 227 | 5.087 | −20.645 | 22.414 | 1.00 | 78.06 | O |
| ATOM | 3729 | CB | LYS | B | 227 | 8.065 | −19.506 | 21.428 | 1.00 | 73.56 | C |
| ATOM | 3730 | CG | LYS | B | 227 | 8.997 | −19.991 | 20.334 | 1.00 | 88.25 | C |
| ATOM | 3731 | CD | LYS | B | 227 | 10.375 | −20.337 | 20.897 | 1.00 | 90.70 | C |
| ATOM | 3732 | CE | LYS | B | 227 | 11.196 | −21.153 | 19.902 | 1.00 | 100.25 | C |
| ATOM | 3733 | NZ | LYS | B | 227 | 12.507 | −21.583 | 20.464 | 1.00 | 95.71 | N |
| ATOM | 3734 | N | GLU | B | 228 | 5.645 | −18.569 | 23.088 | 1.00 | 91.92 | N |
| ATOM | 3735 | CA | GLU | B | 228 | 4.778 | −18.573 | 24.268 | 1.00 | 87.75 | C |
| ATOM | 3736 | C | GLU | B | 228 | 3.318 | −18.715 | 23.880 | 1.00 | 79.54 | C |
| ATOM | 3737 | O | GLU | B | 228 | 2.444 | −18.724 | 24.740 | 1.00 | 88.05 | O |
| ATOM | 3738 | CB | GLU | B | 228 | 4.934 | −17.282 | 25.078 | 1.00 | 91.26 | C |
| ATOM | 3739 | CG | GLU | B | 228 | 6.223 | −17.160 | 25.874 | 1.00 | 109.19 | C |
| ATOM | 3740 | CD | GLU | B | 228 | 6.343 | −15.815 | 26.583 | 1.00 | 119.86 | C |
| ATOM | 3741 | OE1 | GLU | B | 228 | 5.388 | −15.420 | 27.288 | 1.00 | 109.43 | O |
| ATOM | 3742 | OE2 | GLU | B | 228 | 7.395 | −15.153 | 26.435 | 1.00 | 121.55 | O |
| ATOM | 3743 | N | GLN | B | 229 | 3.055 | −18.800 | 22.582 | 1.00 | 94.93 | N |
| ATOM | 3744 | CA | GLN | B | 229 | 1.701 | −19.015 | 22.092 | 1.00 | 91.19 | C |
| ATOM | 3745 | C | GLN | B | 229 | 1.507 | −20.458 | 21.652 | 1.00 | 89.25 | C |
| ATOM | 3746 | O | GLN | B | 229 | 0.405 | −20.996 | 21.751 | 1.00 | 93.53 | O |
| ATOM | 3747 | CB | GLN | B | 229 | 1.382 | −18.070 | 20.934 | 1.00 | 84.10 | C |
| ATOM | 3748 | CG | GLN | B | 229 | 0.954 | −16.680 | 21.370 | 1.00 | 86.84 | C |
| ATOM | 3749 | CD | GLN | B | 229 | 0.451 | −15.833 | 20.213 | 1.00 | 101.43 | C |
| ATOM | 3750 | OE1 | GLN | B | 229 | −0.259 | −14.847 | 20.416 | 1.00 | 93.37 | O |
| ATOM | 3751 | NE2 | GLN | B | 229 | 0.814 | −16.216 | 18.991 | 1.00 | 101.10 | N |
| ATOM | 3752 | N | ILE | B | 230 | 2.589 | −21.080 | 21.190 | 1.00 | 83.18 | N |
| ATOM | 3753 | CA | ILE | B | 230 | 2.534 | −22.385 | 20.540 | 1.00 | 99.88 | C |
| ATOM | 3754 | C | ILE | B | 230 | 3.495 | −22.363 | 19.377 | 1.00 | 108.13 | C |
| ATOM | 3755 | O | ILE | B | 230 | 4.701 | −22.564 | 19.512 | 1.00 | 108.13 | O |
| ATOM | 3756 | CB | ILE | B | 230 | 1.194 | −22.604 | 19.842 | 1.00 | 115.38 | C |
| ATOM | 3757 | CG1 | ILE | B | 230 | 1.265 | −23.850 | 18.965 | 1.00 | 111.77 | C |
| ATOM | 3758 | CG2 | ILE | B | 230 | 0.879 | −21.424 | 18.921 | 1.00 | 112.34 | C |
| ATOM | 3759 | CD1 | ILE | B | 230 | 0.799 | −23.604 | 17.540 | 1.00 | 114.10 | C |
| ATOM | 3760 | N | ARG | B | 267 | −1.226 | −20.535 | 10.457 | 1.00 | 96.06 | N |
| ATOM | 3761 | CA | ARG | B | 267 | −0.491 | −20.193 | 9.249 | 1.00 | 101.47 | C |
| ATOM | 3762 | C | ARG | B | 267 | 0.211 | −18.866 | 9.479 | 1.00 | 106.58 | C |
| ATOM | 3763 | O | ARG | B | 267 | 1.174 | −18.532 | 8.788 | 1.00 | 97.73 | O |
| ATOM | 3764 | CB | ARG | B | 267 | −1.446 | −20.047 | 8.062 | 1.00 | 112.42 | C |
| ATOM | 3765 | CG | ARG | B | 267 | −2.012 | −21.356 | 7.528 | 1.00 | 136.64 | C |
| ATOM | 3766 | CD | ARG | B | 267 | −1.025 | −22.075 | 6.604 | 1.00 | 140.65 | C |
| ATOM | 3767 | NE | ARG | B | 267 | −1.454 | −23.441 | 6.304 | 1.00 | 151.36 | N |
| ATOM | 3768 | CZ | ARG | B | 267 | −0.753 | −24.310 | 5.580 | 1.00 | 137.07 | C |
| ATOM | 3769 | NH1 | ARG | B | 267 | 0.420 | −23.960 | 5.070 | 1.00 | 134.64 | N |
| ATOM | 3770 | NH2 | ARG | B | 267 | −1.226 | −25.531 | 5.366 | 1.00 | 112.52 | N |
| ATOM | 3771 | N | GLU | B | 268 | −0.289 | −18.114 | 10.457 | 1.00 | 112.19 | N |
| ATOM | 3772 | CA | GLU | B | 268 | 0.214 | −16.778 | 10.770 | 1.00 | 98.22 | C |
| ATOM | 3773 | C | GLU | B | 268 | 1.711 | −16.776 | 11.044 | 1.00 | 89.25 | C |
| ATOM | 3774 | O | GLU | B | 268 | 2.383 | −15.762 | 10.867 | 1.00 | 77.50 | O |
| ATOM | 3775 | CB | GLU | B | 268 | −0.522 | −16.206 | 11.984 | 1.00 | 106.43 | C |
| ATOM | 3776 | CG | GLU | B | 268 | −2.037 | −16.287 | 11.894 | 1.00 | 121.11 | C |
| ATOM | 3777 | CD | GLU | B | 268 | −2.606 | −15.421 | 10.785 | 1.00 | 135.87 | C |
| ATOM | 3778 | OE1 | GLU | B | 268 | −1.839 | −14.625 | 10.199 | 1.00 | 136.35 | O |
| ATOM | 3779 | OE2 | GLU | B | 268 | −3.820 | −15.536 | 10.502 | 1.00 | 119.82 | O |
| ATOM | 3780 | N | HIS | B | 269 | 2.229 | −17.916 | 11.483 | 1.00 | 84.80 | N |
| ATOM | 3781 | CA | HIS | B | 269 | 3.646 | −18.034 | 11.786 | 1.00 | 85.68 | C |
| ATOM | 3782 | C | HIS | B | 269 | 4.515 | −18.086 | 10.530 | 1.00 | 79.97 | C |
| ATOM | 3783 | O | HIS | B | 269 | 5.610 | −17.522 | 10.496 | 1.00 | 66.05 | O |
| ATOM | 3784 | CB | HIS | B | 269 | 3.883 | −19.248 | 12.674 | 1.00 | 93.90 | C |
| ATOM | 3785 | CG | HIS | B | 269 | 3.413 | −19.049 | 14.078 | 1.00 | 101.11 | C |
| ATOM | 3786 | ND1 | HIS | B | 269 | 4.181 | −19.378 | 15.175 | 1.00 | 109.47 | N |
| ATOM | 3787 | CD2 | HIS | B | 269 | 2.266 | −18.523 | 14.564 | 1.00 | 100.55 | C |
| ATOM | 3788 | CE1 | HIS | B | 269 | 3.516 | −19.081 | 16.277 | 1.00 | 105.55 | C |
| ATOM | 3789 | NE2 | HIS | B | 269 | 2.352 | −18.559 | 15.935 | 1.00 | 106.90 | N |
| ATOM | 3790 | N | LYS | B | 270 | 4.029 | −18.763 | 9.498 | 1.00 | 86.37 | N |
| ATOM | 3791 | CA | LYS | B | 270 | 4.736 | −18.780 | 8.226 | 1.00 | 84.00 | C |
| ATOM | 3792 | C | LYS | B | 270 | 4.881 | −17.343 | 7.745 | 1.00 | 77.23 | C |
| ATOM | 3793 | O | LYS | B | 270 | 5.943 | −16.946 | 7.259 | 1.00 | 68.91 | O |
| ATOM | 3794 | CB | LYS | B | 270 | 3.993 | −19.639 | 7.199 | 1.00 | 84.96 | C |
| ATOM | 3795 | CG | LYS | B | 270 | 3.904 | −21.104 | 7.605 | 1.00 | 107.28 | C |
| ATOM | 3796 | CD | LYS | B | 270 | 2.896 | −21.876 | 6.771 | 1.00 | 117.71 | C |
| ATOM | 3797 | CE | LYS | B | 270 | 2.635 | −23.252 | 7.372 | 1.00 | 116.08 | C |
| ATOM | 3798 | NZ | LYS | B | 270 | 3.905 | −23.972 | 7.692 | 1.00 | 116.51 | N |
| ATOM | 3799 | N | ALA | B | 271 | 3.814 | −16.563 | 7.907 | 1.00 | 77.57 | N |
| ATOM | 3800 | CA | ALA | B | 271 | 3.839 | −15.145 | 7.559 | 1.00 | 67.05 | C |
| ATOM | 3801 | C | ALA | B | 271 | 4.863 | −14.415 | 8.413 | 1.00 | 55.60 | C |
| ATOM | 3802 | O | ALA | B | 271 | 5.664 | −13.641 | 7.907 | 1.00 | 47.53 | O |
| ATOM | 3803 | CB | ALA | B | 271 | 2.461 | −14.517 | 7.722 | 1.00 | 57.67 | C |
| ATOM | 3804 | N | LEU | B | 272 | 4.844 | −14.667 | 9.714 | 1.00 | 61.55 | N |
| ATOM | 3805 | CA | LEU | B | 272 | 5.838 | −14.060 | 10.587 | 1.00 | 64.62 | C |
| ATOM | 3806 | C | LEU | B | 272 | 7.233 | −14.498 | 10.164 | 1.00 | 54.33 | C |
| ATOM | 3807 | O | LEU | B | 272 | 8.127 | −13.670 | 10.019 | 1.00 | 50.35 | O |
| ATOM | 3808 | CB | LEU | B | 272 | 5.582 | −14.406 | 12.055 | 1.00 | 69.03 | C |
| ATOM | 3809 | CG | LEU | B | 272 | 4.378 | −13.733 | 12.713 | 1.00 | 65.85 | C |
| ATOM | 3810 | CD1 | LEU | B | 272 | 4.467 | −13.888 | 14.217 | 1.00 | 58.80 | C |
| ATOM | 3811 | CD2 | LEU | B | 272 | 4.295 | −12.265 | 12.324 | 1.00 | 51.47 | C |
| ATOM | 3812 | N | LYS | B | 273 | 7.407 | −15.796 | 9.940 | 1.00 | 58.17 | N |
| ATOM | 3813 | CA | LYS | B | 273 | 8.718 | −16.322 | 9.589 | 1.00 | 62.15 | C |
| ATOM | 3814 | C | LYS | B | 273 | 9.270 | −15.662 | 8.326 | 1.00 | 55.97 | C |
| ATOM | 3815 | O | LYS | B | 273 | 10.438 | −15.277 | 8.277 | 1.00 | 52.96 | O |
| ATOM | 3816 | CB | LYS | B | 273 | 8.691 | −17.844 | 9.424 | 1.00 | 66.21 | C |
| ATOM | 3817 | CG | LYS | B | 273 | 10.081 | −18.463 | 9.524 | 1.00 | 74.02 | C |
| ATOM | 3818 | CD | LYS | B | 273 | 10.201 | −19.782 | 8.774 | 1.00 | 70.37 | C |
| ATOM | 3819 | CE | LYS | B | 273 | 11.612 | −20.344 | 8.911 | 1.00 | 74.14 | C |
| ATOM | 3820 | NZ | LYS | B | 273 | 11.847 | −21.513 | 8.020 | 1.00 | 87.37 | N |
| ATOM | 3821 | N | THR | B | 274 | 8.429 | −15.540 | 7.307 | 1.00 | 50.23 | N |
| ATOM | 3822 | CA | THR | B | 274 | 8.819 | −14.846 | 6.088 | 1.00 | 57.86 | C |
| ATOM | 3823 | C | THR | B | 274 | 9.369 | −13.458 | 6.411 | 1.00 | 48.75 | C |
| ATOM | 3824 | O | THR | B | 274 | 10.401 | −13.066 | 5.879 | 1.00 | 41.76 | O |
| ATOM | 3825 | CB | THR | B | 274 | 7.639 | −14.704 | 5.091 | 1.00 | 61.90 | C |
| ATOM | 3826 | OG1 | THR | B | 274 | 7.359 | −15.969 | 4.476 | 1.00 | 65.03 | O |
| ATOM | 3827 | CG2 | THR | B | 274 | 7.972 | −13.693 | 4.009 | 1.00 | 49.82 | C |
| ATOM | 3828 | N | LEU | B | 275 | 8.681 | −12.726 | 7.285 | 1.00 | 43.51 | N |
| ATOM | 3829 | CA | LEU | B | 275 | 9.113 | −11.381 | 7.661 | 1.00 | 44.80 | C |
| ATOM | 3830 | C | LEU | B | 275 | 10.480 | −11.397 | 8.327 | 1.00 | 46.43 | C |
| ATOM | 3831 | O | LEU | B | 275 | 11.296 | −10.511 | 8.093 | 1.00 | 50.55 | O |
| ATOM | 3832 | CB | LEU | B | 275 | 8.101 | −10.705 | 8.582 | 1.00 | 29.73 | C |
| ATOM | 3833 | CG | LEU | B | 275 | 6.699 | −10.550 | 7.991 | 1.00 | 44.30 | C |
| ATOM | 3834 | CD1 | LEU | B | 275 | 5.759 | −9.850 | 8.963 | 1.00 | 32.32 | C |
| ATOM | 3835 | CD2 | LEU | B | 275 | 6.744 | −9.817 | 6.656 | 1.00 | 38.16 | C |
| ATOM | 3836 | N | GLY | B | 276 | 10.730 | −12.406 | 9.154 | 1.00 | 44.84 | N |
| ATOM | 3837 | CA | GLY | B | 276 | 12.011 | −12.531 | 9.824 | 1.00 | 36.72 | C |
| ATOM | 3838 | C | GLY | B | 276 | 13.102 | −12.808 | 8.817 | 1.00 | 43.08 | C |
| ATOM | 3839 | O | GLY | B | 276 | 14.239 | −12.384 | 8.985 | 1.00 | 49.88 | O |
| ATOM | 3840 | N | ILE | B | 277 | 12.749 | −13.525 | 7.759 | 1.00 | 40.22 | N |
| ATOM | 3841 | CA | ILE | B | 277 | 13.701 | −13.835 | 6.705 | 1.00 | 46.75 | C |
| ATOM | 3842 | C | ILE | B | 277 | 14.024 | −12.592 | 5.866 | 1.00 | 44.30 | C |
| ATOM | 3843 | O | ILE | B | 277 | 15.189 | −12.330 | 5.572 | 1.00 | 46.53 | O |
| ATOM | 3844 | CB | ILE | B | 277 | 13.193 | −14.989 | 5.821 | 1.00 | 51.66 | C |
| ATOM | 3845 | CG1 | ILE | B | 277 | 13.145 | −16.287 | 6.635 | 1.00 | 42.99 | C |
| ATOM | 3846 | CG2 | ILE | B | 277 | 14.073 | −15.149 | 4.584 | 1.00 | 35.39 | C |
| ATOM | 3847 | CD1 | ILE | B | 277 | 12.426 | −17.421 | 5.930 | 1.00 | 45.89 | C |
| ATOM | 3848 | N | ILE | B | 278 | 12.990 | −11.841 | 5.488 | 1.00 | 41.57 | N |
| ATOM | 3849 | CA | ILE | B | 278 | 13.146 | −10.525 | 4.871 | 1.00 | 32.71 | C |
| ATOM | 3850 | C | ILE | B | 278 | 14.170 | −9.709 | 5.653 | 1.00 | 43.54 | C |
| ATOM | 3851 | O | ILE | B | 278 | 15.087 | −9.105 | 5.075 | 1.00 | 36.40 | O |
| ATOM | 3852 | CB | ILE | B | 278 | 11.809 | −9.749 | 4.880 | 1.00 | 34.70 | C |
| ATOM | 3853 | CG1 | ILE | B | 278 | 10.792 | −10.408 | 3.955 | 1.00 | 42.37 | C |
| ATOM | 3854 | CG2 | ILE | B | 278 | 11.996 | −8.307 | 4.487 | 1.00 | 39.51 | C |
| ATOM | 3855 | CD1 | ILE | B | 278 | 11.375 | −10.942 | 2.690 | 1.00 | 46.62 | C |
| ATOM | 3856 | N | MET | B | 279 | 14.012 | −9.715 | 6.976 | 1.00 | 37.92 | N |
| ATOM | 3857 | CA | MET | B | 279 | 14.860 | −8.934 | 7.873 | 1.00 | 40.68 | C |
| ATOM | 3858 | C | MET | B | 279 | 16.279 | −9.482 | 7.974 | 1.00 | 42.25 | C |
| ATOM | 3859 | O | MET | B | 279 | 17.240 | −8.718 | 7.924 | 1.00 | 46.61 | O |
| ATOM | 3860 | CB | MET | B | 279 | 14.245 | −8.844 | 9.275 | 1.00 | 43.21 | C |
| ATOM | 3861 | CG | MET | B | 279 | 12.937 | −8.053 | 9.361 | 1.00 | 44.30 | C |
| ATOM | 3862 | SD | MET | B | 279 | 12.231 | −8.091 | 11.036 | 1.00 | 63.24 | S |
| ATOM | 3863 | CE | MET | B | 279 | 10.533 | −7.622 | 10.708 | 1.00 | 35.00 | C |
| ATOM | 3864 | N | GLY | B | 280 | 16.411 | −10.797 | 8.129 | 1.00 | 35.75 | N |
| ATOM | 3865 | CA | GLY | B | 280 | 17.716 | −11.405 | 8.322 | 1.00 | 36.73 | C |
| ATOM | 3866 | C | GLY | B | 280 | 18.584 | −11.305 | 7.080 | 1.00 | 44.40 | C |
| ATOM | 3867 | O | GLY | B | 280 | 19.796 | −11.043 | 7.146 | 1.00 | 32.64 | O |
| ATOM | 3868 | N | VAL | B | 281 | 17.956 | −11.525 | 5.933 | 1.00 | 35.68 | N |
| ATOM | 3869 | CA | VAL | B | 281 | 18.642 | −11.357 | 4.669 | 1.00 | 39.95 | C |
| ATOM | 3870 | C | VAL | B | 281 | 19.064 | −9.899 | 4.512 | 1.00 | 40.12 | C |
| ATOM | 3871 | O | VAL | B | 281 | 20.137 | −9.618 | 3.987 | 1.00 | 34.12 | O |
| ATOM | 3872 | CB | VAL | B | 281 | 17.766 | −11.790 | 3.463 | 1.00 | 47.59 | C |
| ATOM | 3873 | CG1 | VAL | B | 281 | 18.310 | −11.200 | 2.160 | 1.00 | 39.24 | C |
| ATOM | 3874 | CG2 | VAL | B | 281 | 17.694 | −13.302 | 3.367 | 1.00 | 39.47 | C |
| ATOM | 3875 | N | PHE | B | 282 | 18.227 | −8.968 | 4.966 | 1.00 | 37.18 | N |
| ATOM | 3876 | CA | PHE | B | 282 | 18.591 | −7.566 | 4.844 | 1.00 | 32.78 | C |
| ATOM | 3877 | C | PHE | B | 282 | 19.865 | −7.304 | 5.626 | 1.00 | 41.43 | C |
| ATOM | 3878 | O | PHE | B | 282 | 20.791 | −6.658 | 5.127 | 1.00 | 40.28 | O |
| ATOM | 3879 | CB | PHE | B | 282 | 17.486 | −6.638 | 5.338 | 1.00 | 32.12 | C |
| ATOM | 3880 | CG | PHE | B | 282 | 17.860 | −5.183 | 5.274 | 1.00 | 36.54 | C |
| ATOM | 3881 | CD1 | PHE | B | 282 | 17.526 | −4.416 | 4.169 | 1.00 | 35.77 | C |
| ATOM | 3882 | CD2 | PHE | B | 282 | 18.577 | −4.585 | 6.310 | 1.00 | 36.56 | C |
| ATOM | 3883 | CE1 | PHE | B | 282 | 17.882 | −3.076 | 4.100 | 1.00 | 28.88 | C |
| ATOM | 3884 | CE2 | PHE | B | 282 | 18.945 | −3.246 | 6.240 | 1.00 | 30.42 | C |
| ATOM | 3885 | CZ | PHE | B | 282 | 18.594 | −2.490 | 5.138 | 1.00 | 23.23 | C |
| ATOM | 3886 | N | THR | B | 283 | 19.908 | −7.813 | 6.853 | 1.00 | 33.73 | N |
| ATOM | 3887 | CA | THR | B | 283 | 21.026 | −7.553 | 7.746 | 1.00 | 31.18 | C |
| ATOM | 3888 | C | THR | B | 283 | 22.295 | −8.186 | 7.209 | 1.00 | 37.31 | C |
| ATOM | 3889 | O | THR | B | 283 | 23.375 | −7.608 | 7.304 | 1.00 | 41.40 | O |
| ATOM | 3890 | CB | THR | B | 283 | 20.748 | −8.073 | 9.165 | 1.00 | 35.55 | C |
| ATOM | 3891 | OG1 | THR | B | 283 | 19.550 | −7.466 | 9.662 | 1.00 | 41.76 | O |
| ATOM | 3892 | CG2 | THR | B | 283 | 21.900 | −7.739 | 10.104 | 1.00 | 27.01 | C |
| ATOM | 3893 | N | LEU | B | 284 | 22.163 | −9.378 | 6.643 | 1.00 | 42.06 | N |
| ATOM | 3894 | CA | LEU | B | 284 | 23.310 | −10.078 | 6.083 | 1.00 | 42.65 | C |
| ATOM | 3895 | C | LEU | B | 284 | 23.874 | −9.336 | 4.876 | 1.00 | 41.90 | C |
| ATOM | 3896 | O | LEU | B | 284 | 25.086 | −9.202 | 4.736 | 1.00 | 42.14 | O |
| ATOM | 3897 | CB | LEU | B | 284 | 22.918 | −11.504 | 5.703 | 1.00 | 49.08 | C |
| ATOM | 3898 | CG | LEU | B | 284 | 22.992 | −12.500 | 6.860 | 1.00 | 62.77 | C |
| ATOM | 3899 | CD1 | LEU | B | 284 | 22.071 | −13.706 | 6.654 | 1.00 | 46.73 | C |
| ATOM | 3900 | CD2 | LEU | B | 284 | 24.438 | −12.926 | 7.048 | 1.00 | 54.04 | C |
| ATOM | 3901 | N | CYS | B | 285 | 22.984 | −8.845 | 4.020 | 1.00 | 35.56 | N |
| ATOM | 3902 | CA | CYS | B | 285 | 23.367 | −8.177 | 2.783 | 1.00 | 33.98 | C |
| ATOM | 3903 | C | CYS | B | 285 | 23.963 | −6.788 | 2.975 | 1.00 | 37.16 | C |
| ATOM | 3904 | O | CYS | B | 285 | 24.749 | −6.339 | 2.141 | 1.00 | 41.34 | O |
| ATOM | 3905 | CB | CYS | B | 285 | 22.173 | −8.080 | 1.833 | 1.00 | 37.08 | C |
| ATOM | 3906 | SG | CYS | B | 285 | 21.591 | −9.662 | 1.189 | 1.00 | 50.19 | S |
| ATOM | 3907 | N | TRP | B | 286 | 23.595 | −6.105 | 4.055 | 1.00 | 31.92 | N |
| ATOM | 3908 | CA | TRP | B | 286 | 24.061 | −4.735 | 4.265 | 1.00 | 29.35 | C |
| ATOM | 3909 | C | TRP | B | 286 | 25.088 | −4.569 | 5.368 | 1.00 | 32.19 | C |
| ATOM | 3910 | O | TRP | B | 286 | 25.784 | −3.562 | 5.423 | 1.00 | 34.52 | O |
| ATOM | 3911 | CB | TRP | B | 286 | 22.890 | −3.806 | 4.557 | 1.00 | 31.50 | C |
| ATOM | 3912 | CG | TRP | B | 286 | 22.254 | −3.276 | 3.344 | 1.00 | 25.60 | C |
| ATOM | 3913 | CD1 | TRP | B | 286 | 20.997 | −3.537 | 2.909 | 1.00 | 27.81 | C |
| ATOM | 3914 | CD2 | TRP | B | 286 | 22.841 | −2.389 | 2.386 | 1.00 | 28.95 | C |
| ATOM | 3915 | NE1 | TRP | B | 286 | 20.752 | −2.863 | 1.747 | 1.00 | 30.10 | N |
| ATOM | 3916 | CE2 | TRP | B | 286 | 21.870 | −2.151 | 1.396 | 1.00 | 28.81 | C |
| ATOM | 3917 | CE3 | TRP | B | 286 | 24.092 | −1.771 | 2.269 | 1.00 | 31.32 | C |
| ATOM | 3918 | CZ2 | TRP | B | 286 | 22.109 | −1.318 | 0.291 | 1.00 | 23.35 | C |
| ATOM | 3919 | CZ3 | TRP | B | 286 | 24.330 | −0.946 | 1.167 | 1.00 | 29.28 | C |
| ATOM | 3920 | CH2 | TRP | B | 286 | 23.342 | −0.731 | 0.196 | 1.00 | 25.66 | C |
| ATOM | 3921 | N | LEU | B | 287 | 25.180 | −5.540 | 6.261 | 1.00 | 32.90 | N |
| ATOM | 3922 | CA | LEU | B | 287 | 26.043 | −5.358 | 7.413 | 1.00 | 36.95 | C |
| ATOM | 3923 | C | LEU | B | 287 | 27.519 | −5.306 | 7.031 | 1.00 | 40.58 | C |
| ATOM | 3924 | O | LEU | B | 287 | 28.247 | −4.433 | 7.505 | 1.00 | 45.74 | O |
| ATOM | 3925 | CB | LEU | B | 287 | 25.769 | −6.397 | 8.502 | 1.00 | 33.98 | C |
| ATOM | 3926 | CG | LEU | B | 287 | 26.406 | −6.076 | 9.858 | 1.00 | 49.15 | C |
| ATOM | 3927 | CD1 | LEU | B | 287 | 26.106 | −4.636 | 10.309 | 1.00 | 39.22 | C |
| ATOM | 3928 | CD2 | LEU | B | 287 | 25.959 | −7.090 | 10.898 | 1.00 | 41.08 | C |
| ATOM | 3929 | N | PRO | B | 288 | 27.973 | −6.230 | 6.171 | 1.00 | 43.68 | N |
| ATOM | 3930 | CA | PRO | B | 288 | 29.393 | −6.165 | 5.796 | 1.00 | 47.21 | C |
| ATOM | 3931 | C | PRO | B | 288 | 29.804 | −4.751 | 5.396 | 1.00 | 40.01 | C |
| ATOM | 3932 | O | PRO | B | 288 | 30.824 | −4.241 | 5.866 | 1.00 | 35.77 | O |
| ATOM | 3933 | CB | PRO | B | 288 | 29.482 | −7.119 | 4.604 | 1.00 | 32.14 | C |
| ATOM | 3934 | CG | PRO | B | 288 | 28.423 | −8.133 | 4.884 | 1.00 | 38.96 | C |
| ATOM | 3935 | CD | PRO | B | 288 | 27.297 | −7.394 | 5.571 | 1.00 | 37.58 | C |
| ATOM | 3936 | N | PHE | B | 289 | 28.997 | −4.123 | 4.550 | 1.00 | 37.73 | N |
| ATOM | 3937 | CA | PHE | B | 289 | 29.286 | −2.774 | 4.077 | 1.00 | 38.25 | C |
| ATOM | 3938 | C | PHE | B | 289 | 29.400 | −1.747 | 5.217 | 1.00 | 40.34 | C |
| ATOM | 3939 | O | PHE | B | 289 | 30.352 | −0.966 | 5.258 | 1.00 | 39.60 | O |
| ATOM | 3940 | CB | PHE | B | 289 | 28.233 | −2.332 | 3.055 | 1.00 | 31.65 | C |
| ATOM | 3941 | CG | PHE | B | 289 | 28.362 | −0.899 | 2.635 | 1.00 | 31.12 | C |
| ATOM | 3942 | CD1 | PHE | B | 289 | 29.163 | −0.553 | 1.555 | 1.00 | 29.50 | C |
| ATOM | 3943 | CD2 | PHE | B | 289 | 27.678 | 0.108 | 3.318 | 1.00 | 30.72 | C |
| ATOM | 3944 | CE1 | PHE | B | 289 | 29.286 | 0.771 | 1.158 | 1.00 | 27.39 | C |
| ATOM | 3945 | CE2 | PHE | B | 289 | 27.792 | 1.428 | 2.925 | 1.00 | 29.10 | C |
| ATOM | 3946 | CZ | PHE | B | 289 | 28.604 | 1.760 | 1.837 | 1.00 | 27.98 | C |
| ATOM | 3947 | N | PHE | B | 290 | 28.445 | −1.747 | 6.143 | 1.00 | 32.33 | N |
| ATOM | 3948 | CA | PHE | B | 290 | 28.497 | −0.796 | 7.256 | 1.00 | 38.30 | C |
| ATOM | 3949 | C | PHE | B | 290 | 29.599 | −1.095 | 8.265 | 1.00 | 40.74 | C |
| ATOM | 3950 | O | PHE | B | 290 | 30.138 | −0.175 | 8.887 | 1.00 | 40.50 | O |
| ATOM | 3951 | CB | PHE | B | 290 | 27.134 | −0.629 | 7.933 | 1.00 | 29.59 | C |
| ATOM | 3952 | CG | PHE | B | 290 | 26.176 | 0.148 | 7.105 | 1.00 | 28.76 | C |
| ATOM | 3953 | CD1 | PHE | B | 290 | 25.183 | −0.492 | 6.385 | 1.00 | 27.65 | C |
| ATOM | 3954 | CD2 | PHE | B | 290 | 26.308 | 1.522 | 6.989 | 1.00 | 30.86 | C |
| ATOM | 3955 | CE1 | PHE | B | 290 | 24.315 | 0.233 | 5.585 | 1.00 | 28.34 | C |
| ATOM | 3956 | CE2 | PHE | B | 290 | 25.448 | 2.258 | 6.184 | 1.00 | 26.31 | C |
| ATOM | 3957 | CZ | PHE | B | 290 | 24.450 | 1.614 | 5.486 | 1.00 | 26.58 | C |
| ATOM | 3958 | N | LEU | B | 291 | 29.937 | −2.372 | 8.425 | 1.00 | 34.81 | N |
| ATOM | 3959 | CA | LEU | B | 291 | 31.112 | −2.733 | 9.202 | 1.00 | 38.20 | C |
| ATOM | 3960 | C | LEU | B | 291 | 32.345 | −2.115 | 8.552 | 1.00 | 43.44 | C |
| ATOM | 3961 | O | LEU | B | 291 | 33.100 | −1.385 | 9.198 | 1.00 | 37.62 | O |
| ATOM | 3962 | CB | LEU | B | 291 | 31.283 | −4.252 | 9.289 | 1.00 | 44.87 | C |
| ATOM | 3963 | CG | LEU | B | 291 | 30.333 | −5.010 | 10.213 | 1.00 | 55.65 | C |
| ATOM | 3964 | CD1 | LEU | B | 291 | 31.004 | −6.290 | 10.685 | 1.00 | 46.10 | C |
| ATOM | 3965 | CD2 | LEU | B | 291 | 29.941 | −4.149 | 11.397 | 1.00 | 34.59 | C |
| ATOM | 3966 | N | VAL | B | 292 | 32.534 | −2.404 | 7.267 | 1.00 | 38.80 | N |
| ATOM | 3967 | CA | VAL | B | 292 | 33.696 | −1.914 | 6.536 | 1.00 | 46.04 | C |
| ATOM | 3968 | C | VAL | B | 292 | 33.750 | −0.390 | 6.562 | 1.00 | 46.24 | C |
| ATOM | 3969 | O | VAL | B | 292 | 34.823 | 0.213 | 6.568 | 1.00 | 48.97 | O |
| ATOM | 3970 | CB | VAL | B | 292 | 33.714 | −2.435 | 5.083 | 1.00 | 43.91 | C |
| ATOM | 3971 | CG1 | VAL | B | 292 | 34.564 | −1.544 | 4.204 | 1.00 | 54.22 | C |
| ATOM | 3972 | CG2 | VAL | B | 292 | 34.233 | −3.850 | 5.048 | 1.00 | 44.98 | C |
| ATOM | 3973 | N | ASN | B | 293 | 32.578 | 0.226 | 6.594 | 1.00 | 45.15 | N |
| ATOM | 3974 | CA | ASN | B | 293 | 32.479 | 1.671 | 6.621 | 1.00 | 49.65 | C |
| ATOM | 3975 | C | ASN | B | 293 | 33.119 | 2.268 | 7.871 | 1.00 | 49.84 | C |
| ATOM | 3976 | O | ASN | B | 293 | 33.792 | 3.301 | 7.811 | 1.00 | 51.95 | O |
| ATOM | 3977 | CB | ASN | B | 293 | 31.013 | 2.085 | 6.536 | 1.00 | 46.41 | C |
| ATOM | 3978 | CG | ASN | B | 293 | 30.832 | 3.430 | 5.881 | 1.00 | 50.98 | C |
| ATOM | 3979 | OD1 | ASN | B | 293 | 31.401 | 3.699 | 4.823 | 1.00 | 49.29 | O |
| ATOM | 3980 | ND2 | ASN | B | 293 | 30.040 | 4.289 | 6.508 | 1.00 | 52.67 | N |
| ATOM | 3981 | N | ILE | B | 294 | 32.908 | 1.618 | 9.008 | 1.00 | 50.69 | N |
| ATOM | 3982 | CA | ILE | B | 294 | 33.437 | 2.133 | 10.264 | 1.00 | 48.46 | C |
| ATOM | 3983 | C | ILE | B | 294 | 34.924 | 1.836 | 10.430 | 1.00 | 41.51 | C |
| ATOM | 3984 | O | ILE | B | 294 | 35.703 | 2.734 | 10.744 | 1.00 | 33.86 | O |
| ATOM | 3985 | CB | ILE | B | 294 | 32.664 | 1.592 | 11.451 | 1.00 | 44.57 | C |
| ATOM | 3986 | CG1 | ILE | B | 294 | 31.196 | 1.994 | 11.332 | 1.00 | 43.04 | C |
| ATOM | 3987 | CG2 | ILE | B | 294 | 33.254 | 2.135 | 12.740 | 1.00 | 61.37 | C |
| ATOM | 3988 | CD1 | ILE | B | 294 | 30.296 | 1.183 | 12.221 | 1.00 | 51.39 | C |
| ATOM | 3989 | N | VAL | B | 295 | 35.307 | 0.579 | 10.208 | 1.00 | 43.66 | N |
| ATOM | 3990 | CA | VAL | B | 295 | 36.713 | 0.190 | 10.199 | 1.00 | 38.22 | C |
| ATOM | 3991 | C | VAL | B | 295 | 37.551 | 1.227 | 9.465 | 1.00 | 41.50 | C |
| ATOM | 3992 | O | VAL | B | 295 | 38.590 | 1.653 | 9.957 | 1.00 | 45.20 | O |
| ATOM | 3993 | CB | VAL | B | 295 | 36.934 | −1.177 | 9.526 | 1.00 | 32.19 | C |
| ATOM | 3994 | CG1 | VAL | B | 295 | 38.398 | −1.345 | 9.162 | 1.00 | 40.23 | C |
| ATOM | 3995 | CG2 | VAL | B | 295 | 36.494 | −2.301 | 10.439 | 1.00 | 22.56 | C |
| ATOM | 3996 | N | ASN | B | 296 | 37.087 | 1.631 | 8.289 | 1.00 | 38.25 | N |
| ATOM | 3997 | CA | ASN | B | 296 | 37.775 | 2.646 | 7.494 | 1.00 | 50.48 | C |
| ATOM | 3998 | C | ASN | B | 296 | 38.030 | 3.959 | 8.224 | 1.00 | 52.00 | C |
| ATOM | 3999 | O | ASN | B | 296 | 39.084 | 4.579 | 8.057 | 1.00 | 56.60 | O |
| ATOM | 4000 | CB | ASN | B | 296 | 37.008 | 2.929 | 6.199 | 1.00 | 53.54 | C |
| ATOM | 4001 | CG | ASN | B | 296 | 37.769 | 2.503 | 4.965 | 1.00 | 64.71 | C |
| ATOM | 4002 | OD1 | ASN | B | 296 | 38.916 | 2.055 | 5.046 | 1.00 | 69.84 | O |
| ATOM | 4003 | ND2 | ASN | B | 296 | 37.137 | 2.649 | 3.808 | 1.00 | 83.43 | N |
| ATOM | 4004 | N | VAL | B | 297 | 37.054 | 4.396 | 9.012 | 1.00 | 54.20 | N |
| ATOM | 4005 | CA | VAL | B | 297 | 37.185 | 5.646 | 9.748 | 1.00 | 57.14 | C |
| ATOM | 4006 | C | VAL | B | 297 | 38.416 | 5.595 | 10.652 | 1.00 | 60.38 | C |
| ATOM | 4007 | O | VAL | B | 297 | 39.130 | 6.584 | 10.803 | 1.00 | 52.82 | O |
| ATOM | 4008 | CB | VAL | B | 297 | 35.923 | 5.940 | 10.577 | 1.00 | 41.85 | C |
| ATOM | 4009 | CG1 | VAL | B | 297 | 36.091 | 7.219 | 11.368 | 1.00 | 41.12 | C |
| ATOM | 4010 | CG2 | VAL | B | 297 | 34.721 | 6.036 | 9.663 | 1.00 | 50.01 | C |
| ATOM | 4011 | N | PHE | B | 298 | 38.667 | 4.424 | 11.229 | 1.00 | 55.55 | N |
| ATOM | 4012 | CA | PHE | B | 298 | 39.784 | 4.223 | 12.145 | 1.00 | 61.29 | C |
| ATOM | 4013 | C | PHE | B | 298 | 41.107 | 4.092 | 11.399 | 1.00 | 64.30 | C |
| ATOM | 4014 | O | PHE | B | 298 | 42.107 | 4.701 | 11.772 | 1.00 | 73.40 | O |
| ATOM | 4015 | CB | PHE | B | 298 | 39.539 | 2.969 | 12.984 | 1.00 | 56.63 | C |
| ATOM | 4016 | CG | PHE | B | 298 | 38.412 | 3.108 | 13.959 | 1.00 | 61.48 | C |
| ATOM | 4017 | CD1 | PHE | B | 298 | 37.555 | 4.200 | 13.901 | 1.00 | 74.29 | C |
| ATOM | 4018 | CD2 | PHE | B | 298 | 38.191 | 2.139 | 14.922 | 1.00 | 85.08 | C |
| ATOM | 4019 | CE1 | PHE | B | 298 | 36.509 | 4.335 | 14.796 | 1.00 | 69.53 | C |
| ATOM | 4020 | CE2 | PHE | B | 298 | 37.144 | 2.264 | 15.822 | 1.00 | 97.65 | C |
| ATOM | 4021 | CZ | PHE | B | 298 | 36.302 | 3.365 | 15.758 | 1.00 | 84.42 | C |
| ATOM | 4022 | N | ASN | B | 299 | 41.093 | 3.304 | 10.331 | 1.00 | 66.84 | N |
| ATOM | 4023 | CA | ASN | B | 299 | 42.306 | 2.965 | 9.609 | 1.00 | 66.63 | C |
| ATOM | 4024 | C | ASN | B | 299 | 42.070 | 2.816 | 8.114 | 1.00 | 76.14 | C |
| ATOM | 4025 | O | ASN | B | 299 | 41.901 | 1.696 | 7.629 | 1.00 | 75.50 | O |
| ATOM | 4026 | CB | ASN | B | 299 | 42.858 | 1.650 | 10.155 | 1.00 | 76.75 | C |
| ATOM | 4027 | CG | ASN | B | 299 | 44.130 | 1.227 | 9.469 | 1.00 | 69.48 | C |
| ATOM | 4028 | OD1 | ASN | B | 299 | 44.817 | 2.038 | 8.854 | 1.00 | 72.96 | O |
| ATOM | 4029 | ND2 | ASN | B | 299 | 44.459 | −0.051 | 9.580 | 1.00 | 75.80 | N |
| ATOM | 4030 | N | ARG | B | 300 | 42.050 | 3.937 | 7.388 | 1.00 | 93.03 | N |
| ATOM | 4031 | CA | ARG | B | 300 | 41.973 | 3.901 | 5.926 | 1.00 | 73.87 | C |
| ATOM | 4032 | C | ARG | B | 300 | 43.115 | 3.017 | 5.456 | 1.00 | 79.24 | C |
| ATOM | 4033 | O | ARG | B | 300 | 44.187 | 3.034 | 6.057 | 1.00 | 99.16 | O |
| ATOM | 4034 | CB | ARG | B | 300 | 42.108 | 5.307 | 5.311 | 1.00 | 66.81 | C |
| ATOM | 4035 | CG | ARG | B | 300 | 41.261 | 6.398 | 5.987 | 1.00 | 94.31 | C |
| ATOM | 4036 | CD | ARG | B | 300 | 40.857 | 7.526 | 5.020 | 1.00 | 104.58 | C |
| ATOM | 4037 | NE | ARG | B | 300 | 40.058 | 8.574 | 5.669 | 1.00 | 112.59 | N |
| ATOM | 4038 | CZ | ARG | B | 300 | 39.306 | 9.468 | 5.025 | 1.00 | 109.58 | C |
| ATOM | 4039 | NH1 | ARG | B | 300 | 39.228 | 9.452 | 3.700 | 1.00 | 90.81 | N |
| ATOM | 4040 | NH2 | ARG | B | 300 | 38.623 | 10.380 | 5.706 | 1.00 | 100.99 | N |
| ATOM | 4041 | N | ASP | B | 301 | 42.885 | 2.226 | 4.413 | 1.00 | 79.25 | N |
| ATOM | 4042 | CA | ASP | B | 301 | 43.936 | 1.381 | 3.832 | 1.00 | 89.04 | C |
| ATOM | 4043 | C | ASP | B | 301 | 44.203 | 0.082 | 4.595 | 1.00 | 82.82 | C |
| ATOM | 4044 | O | ASP | B | 301 | 45.295 | −0.473 | 4.506 | 1.00 | 87.79 | O |
| ATOM | 4045 | CB | ASP | B | 301 | 45.261 | 2.147 | 3.687 | 1.00 | 84.04 | C |
| ATOM | 4046 | CG | ASP | B | 301 | 45.085 | 3.525 | 3.074 | 1.00 | 104.08 | C |
| ATOM | 4047 | OD1 | ASP | B | 301 | 44.131 | 3.717 | 2.286 | 1.00 | 107.47 | O |
| ATOM | 4048 | OD2 | ASP | B | 301 | 45.915 | 4.413 | 3.382 | 1.00 | 97.26 | O |
| ATOM | 4049 | N | LEU | B | 302 | 43.224 | −0.397 | 5.349 | 1.00 | 75.64 | N |
| ATOM | 4050 | CA | LEU | B | 302 | 43.331 | −1.725 | 5.943 | 1.00 | 74.99 | C |
| ATOM | 4051 | C | LEU | B | 302 | 42.476 | −2.724 | 5.169 | 1.00 | 91.10 | C |
| ATOM | 4052 | O | LEU | B | 302 | 42.692 | −3.937 | 5.244 | 1.00 | 92.93 | O |
| ATOM | 4053 | CB | LEU | B | 302 | 42.884 | −1.723 | 7.395 | 1.00 | 76.08 | C |
| ATOM | 4054 | CG | LEU | B | 302 | 42.825 | −3.163 | 7.901 | 1.00 | 72.25 | C |
| ATOM | 4055 | CD1 | LEU | B | 302 | 44.237 | −3.644 | 8.209 | 1.00 | 81.61 | C |
| ATOM | 4056 | CD2 | LEU | B | 302 | 41.913 | −3.306 | 9.104 | 1.00 | 72.79 | C |
| ATOM | 4057 | N | VAL | B | 303 | 41.490 | −2.208 | 4.441 | 1.00 | 85.48 | N |
| ATOM | 4058 | CA | VAL | B | 303 | 40.637 | −3.035 | 3.595 | 1.00 | 62.71 | C |
| ATOM | 4059 | C | VAL | B | 303 | 40.501 | −2.407 | 2.206 | 1.00 | 69.04 | C |
| ATOM | 4060 | O | VAL | B | 303 | 40.294 | −1.196 | 2.079 | 1.00 | 69.37 | O |
| ATOM | 4061 | CB | VAL | B | 303 | 39.263 | −3.289 | 4.253 | 1.00 | 75.11 | C |
| ATOM | 4062 | CG1 | VAL | B | 303 | 38.226 | −3.700 | 3.219 | 1.00 | 65.87 | C |
| ATOM | 4063 | CG2 | VAL | B | 303 | 39.395 | −4.353 | 5.341 | 1.00 | 77.22 | C |
| ATOM | 4064 | N | PRO | B | 304 | 40.644 | −3.237 | 1.160 | 1.00 | 61.83 | N |
| ATOM | 4065 | CA | PRO | B | 304 | 40.801 | −2.843 | −0.248 | 1.00 | 48.47 | C |
| ATOM | 4066 | C | PRO | B | 304 | 39.559 | −2.252 | −0.917 | 1.00 | 50.91 | C |
| ATOM | 4067 | O | PRO | B | 304 | 38.502 | −2.882 | −0.931 | 1.00 | 48.78 | O |
| ATOM | 4068 | CB | PRO | B | 304 | 41.170 | −4.166 | −0.923 | 1.00 | 45.74 | C |
| ATOM | 4069 | CG | PRO | B | 304 | 40.549 | −5.195 | −0.039 | 1.00 | 47.82 | C |
| ATOM | 4070 | CD | PRO | B | 304 | 40.838 | −4.687 | 1.327 | 1.00 | 54.22 | C |
| ATOM | 4071 | N | ASP | B | 305 | 39.718 | −1.060 | −1.490 | 1.00 | 54.04 | N |
| ATOM | 4072 | CA | ASP | B | 305 | 38.667 | −0.353 | −2.229 | 1.00 | 58.93 | C |
| ATOM | 4073 | C | ASP | B | 305 | 37.717 | −1.255 | −3.019 | 1.00 | 56.48 | C |
| ATOM | 4074 | O | ASP | B | 305 | 36.516 | −0.995 | −3.098 | 1.00 | 51.52 | O |
| ATOM | 4075 | CB | ASP | B | 305 | 39.293 | 0.657 | −3.202 | 1.00 | 70.59 | C |
| ATOM | 4076 | CG | ASP | B | 305 | 39.599 | 2.002 | −2.554 | 1.00 | 96.69 | C |
| ATOM | 4077 | OD1 | ASP | B | 305 | 38.853 | 2.427 | −1.646 | 1.00 | 97.96 | O |
| ATOM | 4078 | OD2 | ASP | B | 305 | 40.584 | 2.649 | −2.974 | 1.00 | 108.96 | O |
| ATOM | 4079 | N | TRP | B | 306 | 38.258 | −2.298 | −3.633 | 1.00 | 55.67 | N |
| ATOM | 4080 | CA | TRP | B | 306 | 37.458 | −3.149 | −4.504 | 1.00 | 53.47 | C |
| ATOM | 4081 | C | TRP | B | 306 | 36.477 | −4.002 | −3.706 | 1.00 | 48.21 | C |
| ATOM | 4082 | O | TRP | B | 306 | 35.399 | −4.331 | −4.199 | 1.00 | 50.15 | O |
| ATOM | 4083 | CB | TRP | B | 306 | 38.356 | −4.028 | −5.376 | 1.00 | 47.81 | C |
| ATOM | 4084 | CG | TRP | B | 306 | 39.122 | −5.025 | −4.597 | 1.00 | 49.03 | C |
| ATOM | 4085 | CD1 | TRP | B | 306 | 40.417 | −4.922 | −4.176 | 1.00 | 52.58 | C |
| ATOM | 4086 | CD2 | TRP | B | 306 | 38.644 | −6.289 | −4.127 | 1.00 | 47.34 | C |
| ATOM | 4087 | NE1 | TRP | B | 306 | 40.776 | −6.050 | −3.474 | 1.00 | 55.27 | N |
| ATOM | 4088 | CE2 | TRP | B | 306 | 39.706 | −6.904 | −3.427 | 1.00 | 50.01 | C |
| ATOM | 4089 | CE3 | TRP | B | 306 | 37.420 | −6.958 | −4.221 | 1.00 | 44.70 | C |
| ATOM | 4090 | CZ2 | TRP | B | 306 | 39.581 | −8.154 | −2.828 | 1.00 | 44.01 | C |
| ATOM | 4091 | CZ3 | TRP | B | 306 | 37.299 | −8.202 | −3.626 | 1.00 | 56.92 | C |
| ATOM | 4092 | CH2 | TRP | B | 306 | 38.374 | −8.787 | −2.939 | 1.00 | 51.66 | C |
| ATOM | 4093 | N | LEU | B | 307 | 36.855 | −4.350 | −2.477 | 1.00 | 41.26 | N |
| ATOM | 4094 | CA | LEU | B | 307 | 35.996 | −5.115 | −1.582 | 1.00 | 41.97 | C |
| ATOM | 4095 | C | LEU | B | 307 | 34.963 | −4.184 | −0.968 | 1.00 | 42.23 | C |
| ATOM | 4096 | O | LEU | B | 307 | 33.851 | −4.584 | −0.619 | 1.00 | 34.18 | O |
| ATOM | 4097 | CB | LEU | B | 307 | 36.624 | −5.763 | −0.480 | 1.00 | 48.22 | C |
| ATOM | 4098 | CG | LEU | B | 307 | 36.036 | −6.521 | 0.587 | 1.00 | 49.79 | C |
| ATOM | 4099 | CD1 | LEU | B | 307 | 35.234 | −7.660 | −0.026 | 1.00 | 43.18 | C |
| ATOM | 4100 | CD2 | LEU | B | 307 | 36.973 | −7.037 | 1.662 | 1.00 | 50.14 | C |
| ATOM | 4101 | N | PHE | B | 308 | 35.351 | −2.926 | −0.834 | 1.00 | 43.47 | N |
| ATOM | 4102 | CA | PHE | B | 308 | 34.411 | −1.892 | −0.469 | 1.00 | 41.35 | C |
| ATOM | 4103 | C | PHE | B | 308 | 33.278 | −1.947 | −1.488 | 1.00 | 38.66 | C |
| ATOM | 4104 | O | PHE | B | 308 | 32.113 | −2.094 | −1.127 | 1.00 | 42.40 | O |
| ATOM | 4105 | CB | PHE | B | 308 | 35.114 | −0.536 | −0.496 | 1.00 | 50.21 | C |
| ATOM | 4106 | CG | PHE | B | 308 | 34.470 | 0.501 | 0.370 | 1.00 | 59.59 | C |
| ATOM | 4107 | CD1 | PHE | B | 308 | 34.804 | 0.605 | 1.707 | 1.00 | 60.05 | C |
| ATOM | 4108 | CD2 | PHE | B | 308 | 33.534 | 1.380 | −0.156 | 1.00 | 75.99 | C |
| ATOM | 4109 | CE1 | PHE | B | 308 | 34.208 | 1.556 | 2.512 | 1.00 | 68.58 | C |
| ATOM | 4110 | CE2 | PHE | B | 308 | 32.935 | 2.342 | 0.644 | 1.00 | 74.46 | C |
| ATOM | 4111 | CZ | PHE | B | 308 | 33.270 | 2.427 | 1.981 | 1.00 | 65.90 | C |
| ATOM | 4112 | N | VAL | B | 309 | 33.631 | −1.863 | −2.768 | 1.00 | 45.30 | N |
| ATOM | 4113 | CA | VAL | B | 309 | 32.645 | −1.896 | −3.847 | 1.00 | 46.64 | C |
| ATOM | 4114 | C | VAL | B | 309 | 31.856 | −3.212 | −3.895 | 1.00 | 43.26 | C |
| ATOM | 4115 | O | VAL | B | 309 | 30.623 | −3.196 | −4.017 | 1.00 | 32.66 | O |
| ATOM | 4116 | CB | VAL | B | 309 | 33.300 | −1.593 | −5.213 | 1.00 | 46.44 | C |
| ATOM | 4117 | CG1 | VAL | B | 309 | 32.341 | −1.900 | −6.350 | 1.00 | 46.73 | C |
| ATOM | 4118 | CG2 | VAL | B | 309 | 33.740 | −0.142 | −5.271 | 1.00 | 32.25 | C |
| ATOM | 4119 | N | ALA | B | 310 | 32.567 | −4.337 | −3.795 | 1.00 | 39.44 | N |
| ATOM | 4120 | CA | ALA | B | 310 | 31.939 | −5.658 | −3.717 | 1.00 | 35.64 | C |
| ATOM | 4121 | C | ALA | B | 310 | 30.790 | −5.686 | −2.716 | 1.00 | 38.80 | C |
| ATOM | 4122 | O | ALA | B | 310 | 29.682 | −6.107 | −3.044 | 1.00 | 30.02 | O |
| ATOM | 4123 | CB | ALA | B | 310 | 32.959 | −6.708 | −3.340 | 1.00 | 31.85 | C |
| ATOM | 4124 | N | PHE | B | 311 | 31.067 | −5.237 | −1.492 | 1.00 | 38.34 | N |
| ATOM | 4125 | CA | PHE | B | 311 | 30.077 | −5.239 | −0.419 | 1.00 | 28.59 | C |
| ATOM | 4126 | C | PHE | B | 311 | 28.886 | −4.312 | −0.679 | 1.00 | 29.02 | C |
| ATOM | 4127 | O | PHE | B | 311 | 27.755 | −4.630 | −0.322 | 1.00 | 29.45 | O |
| ATOM | 4128 | CB | PHE | B | 311 | 30.734 | −4.919 | 0.932 | 1.00 | 28.99 | C |
| ATOM | 4129 | CG | PHE | B | 311 | 31.404 | −6.101 | 1.580 | 1.00 | 33.91 | C |
| ATOM | 4130 | CD1 | PHE | B | 311 | 32.492 | −5.928 | 2.426 | 1.00 | 34.82 | C |
| ATOM | 4131 | CD2 | PHE | B | 311 | 30.955 | −7.392 | 1.329 | 1.00 | 33.09 | C |
| ATOM | 4132 | CE1 | PHE | B | 311 | 33.115 | −7.022 | 3.023 | 1.00 | 40.11 | C |
| ATOM | 4133 | CE2 | PHE | B | 311 | 31.570 | −8.494 | 1.918 | 1.00 | 37.41 | C |
| ATOM | 4134 | CZ | PHE | B | 311 | 32.653 | −8.309 | 2.769 | 1.00 | 42.16 | C |
| ATOM | 4135 | N | ASN | B | 312 | 29.130 | −3.168 | −1.299 | 1.00 | 27.12 | N |
| ATOM | 4136 | CA | ASN | B | 312 | 28.037 | −2.258 | −1.599 | 1.00 | 29.15 | C |
| ATOM | 4137 | C | ASN | B | 312 | 27.079 | −2.866 | −2.644 | 1.00 | 30.87 | C |
| ATOM | 4138 | O | ASN | B | 312 | 25.873 | −2.612 | −2.625 | 1.00 | 27.51 | O |
| ATOM | 4139 | CB | ASN | B | 312 | 28.598 | −0.903 | −2.046 | 1.00 | 27.89 | C |
| ATOM | 4140 | CG | ASN | B | 312 | 27.550 | 0.207 | −2.072 | 1.00 | 30.52 | C |
| ATOM | 4141 | OD1 | ASN | B | 312 | 27.881 | 1.375 | −2.277 | 1.00 | 28.28 | O |
| ATOM | 4142 | ND2 | ASN | B | 312 | 26.285 | −0.153 | −1.879 | 1.00 | 38.95 | N |
| ATOM | 4143 | N | TRP | B | 313 | 27.617 | −3.679 | −3.549 | 1.00 | 33.37 | N |
| ATOM | 4144 | CA | TRP | B | 313 | 26.793 | −4.376 | −4.545 | 1.00 | 30.63 | C |
| ATOM | 4145 | C | TRP | B | 313 | 26.006 | −5.539 | −3.939 | 1.00 | 28.36 | C |
| ATOM | 4146 | O | TRP | B | 313 | 24.968 | −5.951 | −4.471 | 1.00 | 22.30 | O |
| ATOM | 4147 | CB | TRP | B | 313 | 27.634 | −4.826 | −5.755 | 1.00 | 25.72 | C |
| ATOM | 4148 | CG | TRP | B | 313 | 27.804 | −3.719 | −6.725 | 1.00 | 27.45 | C |
| ATOM | 4149 | CD1 | TRP | B | 313 | 28.788 | −2.778 | −6.733 | 1.00 | 30.63 | C |
| ATOM | 4150 | CD2 | TRP | B | 313 | 26.925 | −3.387 | −7.804 | 1.00 | 32.67 | C |
| ATOM | 4151 | NE1 | TRP | B | 313 | 28.589 | −1.887 | −7.762 | 1.00 | 29.11 | N |
| ATOM | 4152 | CE2 | TRP | B | 313 | 27.447 | −2.239 | −8.433 | 1.00 | 35.63 | C |
| ATOM | 4153 | CE3 | TRP | B | 313 | 25.745 | −3.945 | −8.296 | 1.00 | 24.71 | C |
| ATOM | 4154 | CZ2 | TRP | B | 313 | 26.832 | −1.648 | −9.529 | 1.00 | 31.43 | C |
| ATOM | 4155 | CZ3 | TRP | B | 313 | 25.144 | −3.355 | −9.377 | 1.00 | 30.25 | C |
| ATOM | 4156 | CH2 | TRP | B | 313 | 25.688 | −2.220 | −9.984 | 1.00 | 32.80 | C |
| ATOM | 4157 | N | LEU | B | 314 | 26.507 | −6.057 | −2.821 | 1.00 | 25.74 | N |
| ATOM | 4158 | CA | LEU | B | 314 | 25.784 | −7.054 | −2.063 | 1.00 | 25.68 | C |
| ATOM | 4159 | C | LEU | B | 314 | 24.577 | −6.377 | −1.415 | 1.00 | 31.54 | C |
| ATOM | 4160 | O | LEU | B | 314 | 23.503 | −6.958 | −1.306 | 1.00 | 32.21 | O |
| ATOM | 4161 | CB | LEU | B | 314 | 26.686 | −7.688 | −1.014 | 1.00 | 20.61 | C |
| ATOM | 4162 | CG | LEU | B | 314 | 25.933 | −8.727 | −0.181 | 1.00 | 25.27 | C |
| ATOM | 4163 | CD1 | LEU | B | 314 | 25.356 | −9.809 | −1.075 | 1.00 | 25.89 | C |
| ATOM | 4164 | CD2 | LEU | B | 314 | 26.843 | −9.333 | 0.854 | 1.00 | 29.10 | C |
| ATOM | 4165 | N | GLY | B | 315 | 24.754 | −5.129 | −1.003 | 1.00 | 30.86 | N |
| ATOM | 4166 | CA | GLY | B | 315 | 23.643 | −4.358 | −0.489 | 1.00 | 27.54 | C |
| ATOM | 4167 | C | GLY | B | 315 | 22.620 | −4.077 | −1.574 | 1.00 | 28.89 | C |
| ATOM | 4168 | O | GLY | B | 315 | 21.409 | −4.152 | −1.335 | 1.00 | 28.05 | O |
| ATOM | 4169 | N | TYR | B | 316 | 23.106 | −3.733 | −2.765 | 1.00 | 24.69 | N |
| ATOM | 4170 | CA | TYR | B | 316 | 22.218 | −3.440 | −3.881 | 1.00 | 32.67 | C |
| ATOM | 4171 | C | TYR | B | 316 | 21.396 | −4.660 | −4.284 | 1.00 | 35.98 | C |
| ATOM | 4172 | O | TYR | B | 316 | 20.224 | −4.536 | −4.626 | 1.00 | 36.28 | O |
| ATOM | 4173 | CB | TYR | B | 316 | 23.002 | −2.971 | −5.098 | 1.00 | 37.19 | C |
| ATOM | 4174 | CG | TYR | B | 316 | 23.545 | −1.566 | −5.019 | 1.00 | 39.21 | C |
| ATOM | 4175 | CD1 | TYR | B | 316 | 24.654 | −1.196 | −5.776 | 1.00 | 34.11 | C |
| ATOM | 4176 | CD2 | TYR | B | 316 | 22.958 | −0.610 | −4.205 | 1.00 | 31.57 | C |
| ATOM | 4177 | CE1 | TYR | B | 316 | 25.159 | 0.075 | −5.724 | 1.00 | 33.52 | C |
| ATOM | 4178 | CE2 | TYR | B | 316 | 23.469 | 0.678 | −4.145 | 1.00 | 33.06 | C |
| ATOM | 4179 | CZ | TYR | B | 316 | 24.568 | 1.007 | −4.907 | 1.00 | 35.13 | C |
| ATOM | 4180 | OH | TYR | B | 316 | 25.099 | 2.265 | −4.865 | 1.00 | 36.76 | O |
| ATOM | 4181 | N | ALA | B | 317 | 22.028 | −5.832 | −4.265 | 1.00 | 42.14 | N |
| ATOM | 4182 | CA | ALA | B | 317 | 21.381 | −7.078 | −4.683 | 1.00 | 42.55 | C |
| ATOM | 4183 | C | ALA | B | 317 | 20.210 | −7.458 | −3.773 | 1.00 | 41.00 | C |
| ATOM | 4184 | O | ALA | B | 317 | 19.226 | −8.054 | −4.219 | 1.00 | 44.32 | O |
| ATOM | 4185 | CB | ALA | B | 317 | 22.400 | −8.215 | −4.755 | 1.00 | 27.04 | C |
| ATOM | 4186 | N | ASN | B | 318 | 20.321 | −7.121 | −2.495 | 1.00 | 30.05 | N |
| ATOM | 4187 | CA | ASN | B | 318 | 19.213 | −7.312 | −1.591 | 1.00 | 37.27 | C |
| ATOM | 4188 | C | ASN | B | 318 | 17.892 | −6.907 | −2.260 | 1.00 | 33.39 | C |
| ATOM | 4189 | O | ASN | B | 318 | 16.883 | −7.582 | −2.107 | 1.00 | 42.69 | O |
| ATOM | 4190 | CB | ASN | B | 318 | 19.437 | −6.497 | −0.314 | 1.00 | 47.49 | C |
| ATOM | 4191 | CG | ASN | B | 318 | 18.362 | −6.737 | 0.732 | 1.00 | 44.43 | C |
| ATOM | 4192 | OD1 | ASN | B | 318 | 18.403 | −7.735 | 1.461 | 1.00 | 39.86 | O |
| ATOM | 4193 | ND2 | ASN | B | 318 | 17.392 | −5.820 | 0.811 | 1.00 | 31.10 | N |
| ATOM | 4194 | N | SER | B | 319 | 17.908 | −5.818 | −3.015 | 1.00 | 25.28 | N |
| ATOM | 4195 | CA | SER | B | 319 | 16.691 | −5.290 | −3.624 | 1.00 | 31.76 | C |
| ATOM | 4196 | C | SER | B | 319 | 15.993 | −6.272 | −4.551 | 1.00 | 39.75 | C |
| ATOM | 4197 | O | SER | B | 319 | 14.843 | −6.062 | −4.932 | 1.00 | 41.67 | O |
| ATOM | 4198 | CB | SER | B | 319 | 16.972 | −4.000 | −4.392 | 1.00 | 32.97 | C |
| ATOM | 4199 | OG | SER | B | 319 | 17.169 | −2.914 | −3.512 | 1.00 | 37.26 | O |
| ATOM | 4200 | N | ALA | B | 320 | 16.681 | −7.345 | −4.917 | 1.00 | 41.37 | N |
| ATOM | 4201 | CA | ALA | B | 320 | 16.077 | −8.350 | −5.775 | 1.00 | 38.02 | C |
| ATOM | 4202 | C | ALA | B | 320 | 15.660 | −9.606 | −5.000 | 1.00 | 43.74 | C |
| ATOM | 4203 | O | ALA | B | 320 | 14.931 | −10.455 | −5.511 | 1.00 | 45.02 | O |
| ATOM | 4204 | CB | ALA | B | 320 | 17.011 | −8.697 | −6.903 | 1.00 | 33.95 | C |
| ATOM | 4205 | N | MET | B | 321 | 16.107 | −9.715 | −3.756 | 1.00 | 39.48 | N |
| ATOM | 4206 | CA | MET | B | 321 | 15.824 | −10.903 | −2.962 | 1.00 | 49.18 | C |
| ATOM | 4207 | C | MET | B | 321 | 14.395 | −10.983 | −2.412 | 1.00 | 45.35 | C |
| ATOM | 4208 | O | MET | B | 321 | 13.853 | −12.074 | −2.234 | 1.00 | 45.04 | O |
| ATOM | 4209 | CB | MET | B | 321 | 16.861 | −11.050 | −1.848 | 1.00 | 43.74 | C |
| ATOM | 4210 | CG | MET | B | 321 | 18.238 | −11.338 | −2.411 | 1.00 | 48.21 | C |
| ATOM | 4211 | SD | MET | B | 321 | 19.583 | −11.142 | −1.248 | 1.00 | 58.51 | S |
| ATOM | 4212 | CE | MET | B | 321 | 21.010 | −11.259 | −2.335 | 1.00 | 40.14 | C |
| ATOM | 4213 | N | ASN | B | 322 | 13.785 | −9.834 | −2.160 | 1.00 | 47.80 | N |
| ATOM | 4214 | CA | ASN | B | 322 | 12.442 | −9.802 | −1.586 | 1.00 | 53.01 | C |
| ATOM | 4215 | C | ASN | B | 322 | 11.396 | −10.563 | −2.386 | 1.00 | 52.55 | C |
| ATOM | 4216 | O | ASN | B | 322 | 10.763 | −11.479 | −1.858 | 1.00 | 49.35 | O |
| ATOM | 4217 | CB | ASN | B | 322 | 11.969 | −8.363 | −1.372 | 1.00 | 56.63 | C |
| ATOM | 4218 | CG | ASN | B | 322 | 12.400 | −7.813 | −0.052 | 1.00 | 56.12 | C |
| ATOM | 4219 | OD1 | ASN | B | 322 | 13.196 | −8.433 | 0.656 | 1.00 | 37.66 | O |
| ATOM | 4220 | ND2 | ASN | B | 322 | 11.877 | −6.644 | 0.301 | 1.00 | 59.29 | N |
| ATOM | 4221 | N | PRO | B | 323 | 11.187 | −10.169 | −3.655 | 1.00 | 52.48 | N |
| ATOM | 4222 | CA | PRO | B | 323 | 10.149 | −10.855 | −4.427 | 1.00 | 54.93 | C |
| ATOM | 4223 | C | PRO | B | 323 | 10.424 | −12.353 | −4.480 | 1.00 | 48.25 | C |
| ATOM | 4224 | O | PRO | B | 323 | 9.485 | −13.137 | −4.384 | 1.00 | 49.60 | O |
| ATOM | 4225 | CB | PRO | B | 323 | 10.262 | −10.213 | −5.812 | 1.00 | 43.24 | C |
| ATOM | 4226 | CG | PRO | B | 323 | 10.826 | −8.865 | −5.542 | 1.00 | 43.18 | C |
| ATOM | 4227 | CD | PRO | B | 323 | 11.812 | −9.086 | −4.433 | 1.00 | 47.45 | C |
| ATOM | 4228 | N | ILE | B | 324 | 11.689 | −12.740 | −4.602 | 1.00 | 40.45 | N |
| ATOM | 4229 | CA | ILE | B | 324 | 12.053 | −14.150 | −4.534 | 1.00 | 50.35 | C |
| ATOM | 4230 | C | ILE | B | 324 | 11.576 | −14.799 | −3.236 | 1.00 | 55.01 | C |
| ATOM | 4231 | O | ILE | B | 324 | 10.930 | −15.844 | −3.260 | 1.00 | 54.80 | O |
| ATOM | 4232 | CB | ILE | B | 324 | 13.569 | −14.357 | −4.641 | 1.00 | 54.63 | C |
| ATOM | 4233 | CG1 | ILE | B | 324 | 14.014 | −14.268 | −6.102 | 1.00 | 58.01 | C |
| ATOM | 4234 | CG2 | ILE | B | 324 | 13.968 | −15.705 | −4.024 | 1.00 | 38.15 | C |
| ATOM | 4235 | CD1 | ILE | B | 324 | 15.504 | −14.032 | −6.266 | 1.00 | 59.49 | C |
| ATOM | 4236 | N | ILE | B | 325 | 11.900 | −14.180 | −2.104 | 1.00 | 50.59 | N |
| ATOM | 4237 | CA | ILE | B | 325 | 11.526 | −14.727 | −0.809 | 1.00 | 45.25 | C |
| ATOM | 4238 | C | ILE | B | 325 | 10.009 | −14.859 | −0.662 | 1.00 | 54.51 | C |
| ATOM | 4239 | O | ILE | B | 325 | 9.513 | −15.822 | −0.083 | 1.00 | 47.16 | O |
| ATOM | 4240 | CB | ILE | B | 325 | 12.089 | −13.877 | 0.344 | 1.00 | 47.93 | C |
| ATOM | 4241 | CG1 | ILE | B | 325 | 13.610 | −13.810 | 0.261 | 1.00 | 44.81 | C |
| ATOM | 4242 | CG2 | ILE | B | 325 | 11.672 | −14.448 | 1.698 | 1.00 | 50.75 | C |
| ATOM | 4243 | CD1 | ILE | B | 325 | 14.263 | −13.276 | 1.518 | 1.00 | 43.70 | C |
| ATOM | 4244 | N | TYR | B | 326 | 9.271 | −13.891 | −1.192 | 1.00 | 59.16 | N |
| ATOM | 4245 | CA | TYR | B | 326 | 7.816 | −13.928 | −1.097 | 1.00 | 62.17 | C |
| ATOM | 4246 | C | TYR | B | 326 | 7.226 | −15.174 | −1.764 | 1.00 | 65.37 | C |
| ATOM | 4247 | O | TYR | B | 326 | 6.065 | −15.509 | −1.542 | 1.00 | 62.21 | O |
| ATOM | 4248 | CB | TYR | B | 326 | 7.190 | −12.666 | −1.690 | 1.00 | 50.49 | C |
| ATOM | 4249 | CG | TYR | B | 326 | 7.516 | −11.404 | −0.933 | 1.00 | 57.04 | C |
| ATOM | 4250 | CD1 | TYR | B | 326 | 7.624 | −10.187 | −1.596 | 1.00 | 56.32 | C |
| ATOM | 4251 | CD2 | TYR | B | 326 | 7.724 | −11.426 | 0.448 | 1.00 | 54.66 | C |
| ATOM | 4252 | CE1 | TYR | B | 326 | 7.918 | −9.026 | −0.911 | 1.00 | 55.62 | C |
| ATOM | 4253 | CE2 | TYR | B | 326 | 8.024 | −10.265 | 1.145 | 1.00 | 48.65 | C |
| ATOM | 4254 | CZ | TYR | B | 326 | 8.120 | −9.067 | 0.456 | 1.00 | 50.63 | C |
| ATOM | 4255 | OH | TYR | B | 326 | 8.418 | −7.899 | 1.117 | 1.00 | 41.84 | O |
| ATOM | 4256 | N | CYS | B | 327 | 8.029 | −15.856 | −2.575 | 1.00 | 65.19 | N |
| ATOM | 4257 | CA | CYS | B | 327 | 7.586 | −17.076 | −3.252 | 1.00 | 72.20 | C |
| ATOM | 4258 | C | CYS | B | 327 | 7.301 | −18.219 | −2.277 | 1.00 | 75.01 | C |
| ATOM | 4259 | O | CYS | B | 327 | 6.910 | −19.311 | −2.687 | 1.00 | 79.31 | O |
| ATOM | 4260 | CB | CYS | B | 327 | 8.622 | −17.534 | −4.282 | 1.00 | 61.73 | C |
| ATOM | 4261 | SG | CYS | B | 327 | 8.802 | −16.431 | −5.685 | 1.00 | 66.78 | S |
| ATOM | 4262 | N | ARG | B | 328 | 7.508 | −17.974 | −0.990 | 1.00 | 63.43 | N |
| ATOM | 4263 | CA | ARG | B | 328 | 7.196 | −18.974 | 0.017 | 1.00 | 68.93 | C |
| ATOM | 4264 | C | ARG | B | 328 | 5.692 | −19.105 | 0.186 | 1.00 | 79.34 | C |
| ATOM | 4265 | O | ARG | B | 328 | 5.181 | −20.189 | 0.463 | 1.00 | 95.48 | O |
| ATOM | 4266 | CB | ARG | B | 328 | 7.830 | −18.602 | 1.355 | 1.00 | 73.91 | C |
| ATOM | 4267 | CG | ARG | B | 328 | 9.285 | −19.011 | 1.502 | 1.00 | 70.97 | C |
| ATOM | 4268 | CD | ARG | B | 328 | 9.850 | −18.392 | 2.751 | 1.00 | 61.12 | C |
| ATOM | 4269 | NE | ARG | B | 328 | 8.818 | −18.286 | 3.781 | 1.00 | 64.50 | N |
| ATOM | 4270 | CZ | ARG | B | 328 | 8.745 | −19.071 | 4.852 | 1.00 | 76.70 | C |
| ATOM | 4271 | NH1 | ARG | B | 328 | 9.655 | −20.021 | 5.044 | 1.00 | 78.29 | N |
| ATOM | 4272 | NH2 | ARG | B | 328 | 7.768 | −18.902 | 5.735 | 1.00 | 74.08 | N |
| ATOM | 4273 | N | SER | B | 329 | 4.987 | −17.994 | 0.013 | 1.00 | 85.09 | N |
| ATOM | 4274 | CA | SER | B | 329 | 3.549 | −17.959 | 0.234 | 1.00 | 90.96 | C |
| ATOM | 4275 | C | SER | B | 329 | 2.783 | −18.606 | −0.905 | 1.00 | 91.66 | C |
| ATOM | 4276 | O | SER | B | 329 | 3.144 | −18.454 | −2.075 | 1.00 | 75.69 | O |
| ATOM | 4277 | CB | SER | B | 329 | 3.061 | −16.520 | 0.439 | 1.00 | 95.01 | C |
| ATOM | 4278 | OG | SER | B | 329 | 1.672 | −16.480 | 0.728 | 1.00 | 87.75 | O |
| ATOM | 4279 | N | PRO | B | 330 | 1.735 | −19.357 | −0.539 | 1.00 | 110.04 | N |
| ATOM | 4280 | CA | PRO | B | 330 | 0.666 | −19.836 | −1.417 | 1.00 | 110.61 | C |
| ATOM | 4281 | C | PRO | B | 330 | −0.020 | −18.641 | −2.075 | 1.00 | 112.13 | C |
| ATOM | 4282 | O | PRO | B | 330 | −0.065 | −18.560 | −3.304 | 1.00 | 104.27 | O |
| ATOM | 4283 | CB | PRO | B | 330 | −0.295 | −20.527 | −0.444 | 1.00 | 111.18 | C |
| ATOM | 4284 | CG | PRO | B | 330 | 0.562 | −20.937 | 0.710 | 1.00 | 102.08 | C |
| ATOM | 4285 | CD | PRO | B | 330 | 1.591 | −19.859 | 0.841 | 1.00 | 101.96 | C |
| ATOM | 4286 | N | ASP | B | 331 | −0.537 | −17.728 | −1.251 | 1.00 | 111.51 | N |
| ATOM | 4287 | CA | ASP | B | 331 | −1.109 | −16.467 | −1.721 | 1.00 | 110.54 | C |
| ATOM | 4288 | C | ASP | B | 331 | −0.257 | −15.790 | −2.785 | 1.00 | 113.28 | C |
| ATOM | 4289 | O | ASP | B | 331 | −0.674 | −15.665 | −3.938 | 1.00 | 114.79 | O |
| ATOM | 4290 | CB | ASP | B | 331 | −1.312 | −15.499 | −0.553 | 1.00 | 112.25 | C |
| ATOM | 4291 | CG | ASP | B | 331 | −2.771 | −15.246 | −0.259 | 1.00 | 127.64 | C |
| ATOM | 4292 | OD1 | ASP | B | 331 | −3.620 | −15.882 | −0.915 | 1.00 | 135.05 | O |
| ATOM | 4293 | OD2 | ASP | B | 331 | −3.069 | −14.406 | 0.617 | 1.00 | 137.73 | O |
| ATOM | 4294 | N | PHE | B | 332 | 0.932 | −15.343 | −2.392 | 1.00 | 111.66 | N |
| ATOM | 4295 | CA | PHE | B | 332 | 1.837 | −14.685 | −3.328 | 1.00 | 108.21 | C |
| ATOM | 4296 | C | PHE | B | 332 | 2.209 | −15.598 | −4.505 | 1.00 | 111.38 | C |
| ATOM | 4297 | O | PHE | B | 332 | 2.522 | −15.119 | −5.594 | 1.00 | 109.32 | O |
| ATOM | 4298 | CB | PHE | B | 332 | 3.091 | −14.168 | −2.610 | 1.00 | 101.50 | C |
| ATOM | 4299 | CG | PHE | B | 332 | 2.887 | −12.860 | −1.877 | 1.00 | 94.15 | C |
| ATOM | 4300 | CD1 | PHE | B | 332 | 3.517 | −12.624 | −0.658 | 1.00 | 89.30 | C |
| ATOM | 4301 | CD2 | PHE | B | 332 | 2.068 | −11.871 | −2.407 | 1.00 | 75.63 | C |
| ATOM | 4302 | CE1 | PHE | B | 332 | 3.341 | −11.425 | 0.017 | 1.00 | 65.14 | C |
| ATOM | 4303 | CE2 | PHE | B | 332 | 1.887 | −10.675 | −1.742 | 1.00 | 77.85 | C |
| ATOM | 4304 | CZ | PHE | B | 332 | 2.525 | −10.451 | −0.527 | 1.00 | 86.15 | C |
| ATOM | 4305 | N | ARG | B | 333 | 2.157 | −16.910 | −4.289 | 1.00 | 110.44 | N |
| ATOM | 4306 | CA | ARG | B | 333 | 2.428 | −17.867 | −5.358 | 1.00 | 107.49 | C |
| ATOM | 4307 | C | ARG | B | 333 | 1.306 | −17.862 | −6.398 | 1.00 | 102.94 | C |
| ATOM | 4308 | O | ARG | B | 333 | 1.567 | −17.856 | −7.598 | 1.00 | 106.52 | O |
| ATOM | 4309 | CB | ARG | B | 333 | 2.610 | −19.270 | −4.781 | 1.00 | 100.76 | C |
| ATOM | 4310 | CG | ARG | B | 333 | 3.538 | −20.171 | −5.579 | 1.00 | 100.36 | C |
| ATOM | 4311 | CD | ARG | B | 333 | 4.131 | −21.228 | −4.668 | 1.00 | 90.01 | C |
| ATOM | 4312 | NE | ARG | B | 333 | 3.176 | −21.620 | −3.635 | 1.00 | 99.45 | N |
| ATOM | 4313 | CZ | ARG | B | 333 | 3.509 | −21.949 | −2.391 | 1.00 | 101.47 | C |
| ATOM | 4314 | NH1 | ARG | B | 333 | 4.783 | −21.931 | −2.015 | 1.00 | 86.95 | N |
| ATOM | 4315 | NH2 | ARG | B | 333 | 2.568 | −22.288 | −1.517 | 1.00 | 88.95 | N |
| ATOM | 4316 | N | LYS | B | 334 | 0.061 | −17.853 | −5.931 | 1.00 | 99.16 | N |
| ATOM | 4317 | CA | LYS | B | 334 | −1.099 | −17.858 | −6.820 | 1.00 | 102.01 | C |
| ATOM | 4318 | C | LYS | B | 334 | −1.338 | −16.493 | −7.466 | 1.00 | 111.95 | C |
| ATOM | 4319 | O | LYS | B | 334 | −2.040 | −16.396 | −8.471 | 1.00 | 119.51 | O |
| ATOM | 4320 | CB | LYS | B | 334 | −2.363 | −18.281 | −6.066 | 1.00 | 112.35 | C |
| ATOM | 4321 | CG | LYS | B | 334 | −2.208 | −19.496 | −5.164 | 1.00 | 112.05 | C |
| ATOM | 4322 | CD | LYS | B | 334 | −3.257 | −19.468 | −4.056 | 1.00 | 110.60 | C |
| ATOM | 4323 | CE | LYS | B | 334 | −3.161 | −20.675 | −3.138 | 1.00 | 101.29 | C |
| ATOM | 4324 | NZ | LYS | B | 334 | −4.270 | −20.670 | −2.140 | 1.00 | 97.06 | N |
| ATOM | 4325 | N | ALA | B | 335 | −0.771 | −15.441 | −6.881 | 1.00 | 110.97 | N |
| ATOM | 4326 | CA | ALA | B | 335 | −0.897 | −14.093 | −7.439 | 1.00 | 111.79 | C |
| ATOM | 4327 | C | ALA | B | 335 | 0.280 | −13.751 | −8.361 | 1.00 | 121.97 | C |
| ATOM | 4328 | O | ALA | B | 335 | 0.167 | −12.885 | −9.234 | 1.00 | 109.59 | O |
| ATOM | 4329 | CB | ALA | B | 335 | −1.037 | −13.052 | −6.325 | 1.00 | 75.15 | C |
| ATOM | 4330 | N | PHE | B | 336 | 1.406 | −14.436 | −8.160 | 1.00 | 120.87 | N |
| ATOM | 4331 | CA | PHE | B | 336 | 2.586 | −14.255 | −9.006 | 1.00 | 119.16 | C |
| ATOM | 4332 | C | PHE | B | 336 | 2.355 | −14.854 | −10.398 | 1.00 | 118.29 | C |
| ATOM | 4333 | O | PHE | B | 336 | 2.956 | −14.417 | −11.380 | 1.00 | 102.70 | O |
| ATOM | 4334 | CB | PHE | B | 336 | 3.835 | −14.897 | −8.368 | 1.00 | 115.86 | C |
| ATOM | 4335 | CG | PHE | B | 336 | 4.493 | −14.055 | −7.287 | 1.00 | 124.23 | C |
| ATOM | 4336 | CD1 | PHE | B | 336 | 4.390 | −12.667 | −7.290 | 1.00 | 119.81 | C |
| ATOM | 4337 | CD2 | PHE | B | 336 | 5.249 | −14.660 | −6.285 | 1.00 | 109.51 | C |
| ATOM | 4338 | CE1 | PHE | B | 336 | 5.006 | −11.900 | −6.299 | 1.00 | 93.03 | C |
| ATOM | 4339 | CE2 | PHE | B | 336 | 5.866 | −13.901 | −5.296 | 1.00 | 83.46 | C |
| ATOM | 4340 | CZ | PHE | B | 336 | 5.744 | −12.521 | −5.305 | 1.00 | 77.61 | C |
| ATOM | 4341 | N | LYS | B | 337 | 1.480 | −15.855 | −10.469 | 1.00 | 120.39 | N |
| ATOM | 4342 | CA | LYS | B | 337 | 1.248 | −16.599 | −11.706 | 1.00 | 122.08 | C |
| ATOM | 4343 | C | LYS | B | 337 | 0.026 | −16.108 | −12.482 | 1.00 | 121.81 | C |
| ATOM | 4344 | O | LYS | B | 337 | −0.157 | −16.470 | −13.643 | 1.00 | 125.11 | O |
| ATOM | 4345 | CB | LYS | B | 337 | 1.121 | −18.099 | −11.414 | 1.00 | 111.45 | C |
| ATOM | 4346 | CG | LYS | B | 337 | 2.332 | −18.691 | −10.705 | 1.00 | 106.49 | C |
| ATOM | 4347 | CD | LYS | B | 337 | 1.984 | −19.982 | −9.981 | 1.00 | 113.20 | C |
| ATOM | 4348 | CE | LYS | B | 337 | 3.102 | −20.403 | −9.031 | 1.00 | 123.56 | C |
| ATOM | 4349 | NZ | LYS | B | 337 | 2.733 | −21.608 | −8.224 | 1.00 | 100.74 | N |
| ATOM | 4350 | N | ARG | B | 338 | −0.807 | −15.291 | −11.843 | 1.00 | 121.29 | N |
| ATOM | 4351 | CA | ARG | B | 338 | −1.979 | −14.716 | −12.508 | 1.00 | 121.70 | C |
| ATOM | 4352 | C | ARG | B | 338 | −1.600 | −13.561 | −13.439 | 1.00 | 127.12 | C |
| ATOM | 4353 | O | ARG | B | 338 | −2.242 | −13.341 | −14.470 | 1.00 | 121.10 | O |
| ATOM | 4354 | CB | ARG | B | 338 | −2.998 | −14.221 | −11.478 | 1.00 | 122.50 | C |
| ATOM | 4355 | CG | ARG | B | 338 | −3.700 | −15.314 | −10.700 | 1.00 | 130.70 | C |
| ATOM | 4356 | CD | ARG | B | 338 | −4.620 | −14.713 | −9.648 | 1.00 | 137.60 | C |
| ATOM | 4357 | NE | ARG | B | 338 | −5.348 | −15.730 | −8.896 | 1.00 | 142.59 | N |
| ATOM | 4358 | CZ | ARG | B | 338 | −6.368 | −15.467 | −8.085 | 1.00 | 153.95 | C |
| ATOM | 4359 | NH1 | ARG | B | 338 | −6.781 | −14.216 | −7.924 | 1.00 | 145.87 | N |
| ATOM | 4360 | NH2 | ARG | B | 338 | −6.978 | −16.452 | −7.437 | 1.00 | 162.50 | N |
| ATOM | 4361 | N | LEU | B | 339 | −0.560 | −12.822 | −13.058 | 1.00 | 128.05 | N |
| ATOM | 4362 | CA | LEU | B | 339 | −0.108 | −11.652 | −13.811 | 1.00 | 120.13 | C |
| ATOM | 4363 | C | LEU | B | 339 | 0.942 | −12.050 | −14.854 | 1.00 | 121.90 | C |
| ATOM | 4364 | O | LEU | B | 339 | 1.074 | −11.407 | −15.899 | 1.00 | 105.79 | O |
| ATOM | 4365 | CB | LEU | B | 339 | 0.453 | −10.594 | −12.852 | 1.00 | 106.20 | C |
| ATOM | 4366 | CG | LEU | B | 339 | −0.436 | −10.197 | −11.663 | 1.00 | 96.28 | C |
| ATOM | 4367 | CD1 | LEU | B | 339 | 0.345 | −10.169 | −10.352 | 1.00 | 79.91 | C |
| ATOM | 4368 | CD2 | LEU | B | 339 | −1.130 | −8.869 | −11.910 | 1.00 | 74.91 | C |
| ATOM | 4369 | N | LEU | B | 340 | 1.687 | −13.114 | −14.559 | 1.00 | 128.17 | N |
| ATOM | 4370 | CA | LEU | B | 340 | 2.622 | −13.703 | −15.517 | 1.00 | 133.42 | C |
| ATOM | 4371 | C | LEU | B | 340 | 1.874 | −14.658 | −16.466 | 1.00 | 139.47 | C |
| ATOM | 4372 | O | LEU | B | 340 | 2.485 | −15.321 | −17.309 | 1.00 | 131.04 | O |
| ATOM | 4373 | CB | LEU | B | 340 | 3.765 | −14.434 | −14.787 | 1.00 | 127.90 | C |
| ATOM | 4374 | CG | LEU | B | 340 | 4.846 | −13.629 | −14.043 | 1.00 | 105.66 | C |
| ATOM | 4375 | CD1 | LEU | B | 340 | 5.413 | −14.415 | −12.862 | 1.00 | 86.15 | C |
| ATOM | 4376 | CD2 | LEU | B | 340 | 5.970 | −13.186 | −14.981 | 1.00 | 78.32 | C |
| ATOM | 4377 | N | ALA | B | 341 | 0.550 | −14.717 | −16.307 | 1.00 | 135.84 | N |
| ATOM | 4378 | CA | ALA | B | 341 | −0.345 | −15.507 | −17.165 | 1.00 | 126.94 | C |
| ATOM | 4379 | C | ALA | B | 341 | 0.012 | −16.996 | −17.297 | 1.00 | 143.65 | C |
| ATOM | 4380 | O | ALA | B | 341 | 0.823 | −17.377 | −18.145 | 1.00 | 137.53 | O |
| ATOM | 4381 | CB | ALA | B | 341 | −0.475 | −14.859 | −18.542 | 1.00 | 117.07 | C |
| ATOM | 4382 | N | PHE | B | 342 | −0.615 | −17.828 | −16.464 | 1.00 | 148.59 | N |
| ATOM | 4383 | CA | PHE | B | 342 | −0.409 | −19.279 | −16.495 | 1.00 | 144.33 | C |
| ATOM | 4384 | C | PHE | B | 342 | −1.727 | −20.048 | −16.445 | 1.00 | 130.89 | C |
| ATOM | 4385 | O | PHE | B | 342 | −1.807 | −21.126 | −15.851 | 1.00 | 117.74 | O |
| ATOM | 4386 | CB | PHE | B | 342 | 0.488 | −19.730 | −15.338 | 1.00 | 141.69 | C |
| ATOM | 4387 | CG | PHE | B | 342 | 1.955 | −19.564 | −15.606 | 1.00 | 137.50 | C |
| ATOM | 4388 | CD1 | PHE | B | 342 | 2.585 | −18.353 | −15.364 | 1.00 | 143.13 | C |
| ATOM | 4389 | CD2 | PHE | B | 342 | 2.704 | −20.618 | −16.098 | 1.00 | 143.09 | C |
| ATOM | 4390 | CE1 | PHE | B | 342 | 3.935 | −18.195 | −15.611 | 1.00 | 137.82 | C |
| ATOM | 4391 | CE2 | PHE | B | 342 | 4.054 | −20.470 | −16.346 | 1.00 | 152.37 | C |
| ATOM | 4392 | CZ | PHE | B | 342 | 4.672 | −19.254 | −16.102 | 1.00 | 150.97 | C |
| ATOM | 4393 | C16 | PDL | B | 400 | 29.184 | 7.069 | 4.937 | 1.00 | 38.38 | C |
| ATOM | 4394 | N3 | PDL | B | 400 | 30.198 | 7.533 | 5.152 | 1.00 | 36.99 | N |
| ATOM | 4395 | N1 | PDL | B | 400 | 26.640 | 6.887 | 5.573 | 1.00 | 30.93 | N |
| ATOM | 4396 | C1 | PDL | B | 400 | 27.850 | 6.583 | 4.719 | 1.00 | 41.26 | C |
| ATOM | 4397 | C2 | PDL | B | 400 | 27.442 | 5.605 | 3.626 | 1.00 | 25.18 | C |
| ATOM | 4398 | C3 | PDL | B | 400 | 26.003 | 5.338 | 3.817 | 1.00 | 27.64 | C |
| ATOM | 4399 | C4 | PDL | B | 400 | 25.030 | 4.457 | 3.065 | 1.00 | 33.14 | C |
| ATOM | 4400 | C5 | PDL | B | 400 | 23.560 | 4.358 | 3.477 | 1.00 | 30.90 | C |
| ATOM | 4401 | C6 | PDL | B | 400 | 23.064 | 5.159 | 4.681 | 1.00 | 32.86 | C |
| ATOM | 4402 | C7 | PDL | B | 400 | 24.036 | 6.038 | 5.444 | 1.00 | 34.59 | C |
| ATOM | 4403 | C8 | PDL | B | 400 | 25.503 | 6.122 | 5.016 | 1.00 | 30.42 | C |
| ATOM | 4404 | O1 | PDL | B | 400 | 25.519 | 3.723 | 1.989 | 1.00 | 35.41 | O |
| ATOM | 4405 | C9 | PDL | B | 400 | 24.720 | 3.588 | 0.854 | 1.00 | 35.15 | C |
| ATOM | 4406 | C10 | PDL | B | 400 | 25.620 | 2.952 | −0.198 | 1.00 | 27.24 | C |
| ATOM | 4407 | O2 | PDL | B | 400 | 24.804 | 2.393 | −1.197 | 1.00 | 31.27 | O |
| ATOM | 4408 | C11 | PDL | B | 400 | 26.522 | 4.071 | −0.743 | 1.00 | 23.86 | C |
| ATOM | 4409 | N2 | PDL | B | 400 | 26.911 | 3.874 | −2.133 | 1.00 | 36.17 | N |
| ATOM | 4410 | C12 | PDL | B | 400 | 27.783 | 4.976 | −2.559 | 1.00 | 34.60 | C |
| ATOM | 4411 | C13 | PDL | B | 400 | 28.937 | 5.154 | −1.541 | 1.00 | 16.62 | C |
| ATOM | 4412 | C14 | PDL | B | 400 | 26.994 | 6.311 | −2.646 | 1.00 | 28.02 | C |
| ATOM | 4413 | C15 | PDL | B | 400 | 28.316 | 4.566 | −3.955 | 1.00 | 28.75 | C |
| ATOM | 4414 | NA | NA | B | 401 | 33.452 | 14.952 | −8.392 | 1.00 | 48.82 | Na |

TABLE C

| | | | | | | | |
|---|---|---|---|---|---|---|---|
| CRYST1 | 55.500 | 86.800 | 95.500 | 67.60 | 73.30 | 85.80 | P 1 |

| | | | | |
|---|---|---|---|---|
| SCALE1 | 0.018018 | −0.001323 | −0.005298 | 0.00000 |
| SCALE2 | 0.000000 | 0.011552 | −0.004700 | 0.00000 |
| SCALE3 | 0.000000 | 0.000000 | 0.011803 | 0.00000 |

| | | | | | | | | | | | |
|---|---|---|---|---|---|---|---|---|---|---|---|
| ATOM | 4415 | N | GLN | C | 31 | 62.786 | 46.162 | 14.725 | 1.00 | 73.97 | N |
| ATOM | 4416 | CA | GLN | C | 31 | 62.534 | 44.982 | 13.901 | 1.00 | 84.04 | C |
| ATOM | 4417 | C | GLN | C | 31 | 62.029 | 45.362 | 12.511 | 1.00 | 79.46 | C |
| ATOM | 4418 | O | GLN | C | 31 | 60.883 | 45.781 | 12.355 | 1.00 | 80.45 | O |
| ATOM | 4419 | CB | GLN | C | 31 | 61.515 | 44.068 | 14.578 | 1.00 | 65.74 | C |
| ATOM | 4420 | CG | GLN | C | 31 | 61.854 | 43.733 | 16.022 | 1.00 | 81.81 | C |
| ATOM | 4421 | CD | GLN | C | 31 | 60.811 | 42.840 | 16.663 | 1.00 | 78.61 | C |
| ATOM | 4422 | OE1 | GLN | C | 31 | 60.080 | 42.128 | 15.971 | 1.00 | 83.04 | O |
| ATOM | 4423 | NE2 | GLN | C | 31 | 60.733 | 42.873 | 17.989 | 1.00 | 53.86 | N |
| ATOM | 4424 | N | TRP | C | 32 | 62.876 | 45.202 | 11.499 | 1.00 | 80.99 | N |
| ATOM | 4425 | CA | TRP | C | 32 | 62.469 | 45.527 | 10.143 | 1.00 | 66.35 | C |
| ATOM | 4426 | C | TRP | C | 32 | 61.316 | 44.607 | 9.725 | 1.00 | 71.15 | C |
| ATOM | 4427 | O | TRP | C | 32 | 60.527 | 44.950 | 8.848 | 1.00 | 76.04 | O |
| ATOM | 4428 | CB | TRP | C | 32 | 63.668 | 45.409 | 9.205 | 1.00 | 60.12 | C |
| ATOM | 4429 | CG | TRP | C | 32 | 63.348 | 45.401 | 7.752 | 1.00 | 81.94 | C |
| ATOM | 4430 | CD1 | TRP | C | 32 | 63.301 | 46.481 | 6.915 | 1.00 | 88.94 | C |
| ATOM | 4431 | CD2 | TRP | C | 32 | 63.067 | 44.249 | 6.941 | 1.00 | 84.57 | C |
| ATOM | 4432 | NE1 | TRP | C | 32 | 62.993 | 46.073 | 5.637 | 1.00 | 99.22 | N |
| ATOM | 4433 | CE2 | TRP | C | 32 | 62.843 | 44.709 | 5.625 | 1.00 | 96.78 | C |
| ATOM | 4434 | CE3 | TRP | C | 32 | 62.970 | 42.877 | 7.205 | 1.00 | 65.06 | C |
| ATOM | 4435 | CZ2 | TRP | C | 32 | 62.526 | 43.840 | 4.568 | 1.00 | 78.58 | C |
| ATOM | 4436 | CZ3 | TRP | C | 32 | 62.657 | 42.017 | 6.158 | 1.00 | 66.47 | C |
| ATOM | 4437 | CH2 | TRP | C | 32 | 62.438 | 42.504 | 4.855 | 1.00 | 66.98 | C |
| ATOM | 4438 | N | GLU | C | 33 | 61.215 | 43.450 | 10.377 | 1.00 | 68.90 | N |
| ATOM | 4439 | CA | GLU | C | 33 | 60.117 | 42.510 | 10.151 | 1.00 | 59.18 | C |
| ATOM | 4440 | C | GLU | C | 33 | 58.768 | 43.096 | 10.535 | 1.00 | 58.54 | C |
| ATOM | 4441 | O | GLU | C | 33 | 57.749 | 42.745 | 9.954 | 1.00 | 60.57 | O |
| ATOM | 4442 | CB | GLU | C | 33 | 60.324 | 41.214 | 10.949 | 1.00 | 59.21 | C |
| ATOM | 4443 | CG | GLU | C | 33 | 59.060 | 40.340 | 11.045 | 1.00 | 62.28 | C |
| ATOM | 4444 | CD | GLU | C | 33 | 59.191 | 39.145 | 11.997 | 1.00 | 78.60 | C |
| ATOM | 4445 | OE1 | GLU | C | 33 | 60.246 | 39.002 | 12.653 | 1.00 | 81.38 | O |
| ATOM | 4446 | OE2 | GLU | C | 33 | 58.229 | 38.342 | 12.090 | 1.00 | 63.82 | O |
| ATOM | 4447 | N | ALA | C | 34 | 58.759 | 43.979 | 11.526 | 1.00 | 71.57 | N |
| ATOM | 4448 | CA | ALA | C | 34 | 57.508 | 44.516 | 12.063 | 1.00 | 65.77 | C |
| ATOM | 4449 | C | ALA | C | 34 | 56.901 | 45.614 | 11.187 | 1.00 | 70.86 | C |
| ATOM | 4450 | O | ALA | C | 34 | 55.682 | 45.663 | 11.004 | 1.00 | 68.62 | O |
| ATOM | 4451 | CB | ALA | C | 34 | 57.712 | 45.019 | 13.478 | 1.00 | 63.35 | C |
| ATOM | 4452 | N | GLY | C | 35 | 57.745 | 46.494 | 10.651 | 1.00 | 66.41 | N |
| ATOM | 4453 | CA | GLY | C | 35 | 57.276 | 47.540 | 9.759 | 1.00 | 52.59 | C |
| ATOM | 4454 | C | GLY | C | 35 | 56.802 | 46.950 | 8.446 | 1.00 | 59.86 | C |
| ATOM | 4455 | O | GLY | C | 35 | 55.877 | 47.456 | 7.813 | 1.00 | 60.33 | O |
| ATOM | 4456 | N | MET | C | 36 | 57.449 | 45.865 | 8.040 | 1.00 | 60.06 | N |
| ATOM | 4457 | CA | MET | C | 36 | 57.107 | 45.173 | 6.808 | 1.00 | 56.81 | C |
| ATOM | 4458 | C | MET | C | 36 | 55.742 | 44.484 | 6.947 | 1.00 | 62.32 | C |
| ATOM | 4459 | O | MET | C | 36 | 54.906 | 44.555 | 6.048 | 1.00 | 66.44 | O |
| ATOM | 4460 | CB | MET | C | 36 | 58.213 | 44.174 | 6.449 | 1.00 | 54.32 | C |
| ATOM | 4461 | CG | MET | C | 36 | 58.453 | 43.990 | 4.949 | 1.00 | 83.69 | C |
| ATOM | 4462 | SD | MET | C | 36 | 58.945 | 45.492 | 4.053 | 1.00 | 79.58 | S |
| ATOM | 4463 | CE | MET | C | 36 | 59.989 | 46.285 | 5.275 | 1.00 | 78.14 | C |
| ATOM | 4464 | N | SER | C | 37 | 55.510 | 43.842 | 8.088 | 1.00 | 62.46 | N |
| ATOM | 4465 | CA | SER | C | 37 | 54.224 | 43.208 | 8.372 | 1.00 | 50.69 | C |
| ATOM | 4466 | C | SER | C | 37 | 53.137 | 44.256 | 8.544 | 1.00 | 55.16 | C |
| ATOM | 4467 | O | SER | C | 37 | 51.961 | 43.929 | 8.705 | 1.00 | 51.21 | O |
| ATOM | 4468 | CB | SER | C | 37 | 54.304 | 42.361 | 9.646 | 1.00 | 50.62 | C |
| ATOM | 4469 | OG | SER | C | 37 | 55.345 | 41.402 | 9.581 | 1.00 | 58.02 | O |
| ATOM | 4470 | N | LEU | C | 38 | 53.532 | 45.521 | 8.532 | 1.00 | 66.77 | N |
| ATOM | 4471 | CA | LEU | C | 38 | 52.566 | 46.599 | 8.665 | 1.00 | 62.24 | C |
| ATOM | 4472 | C | LEU | C | 38 | 52.107 | 47.083 | 7.290 | 1.00 | 60.02 | C |
| ATOM | 4473 | O | LEU | C | 38 | 50.908 | 47.096 | 7.011 | 1.00 | 57.78 | O |
| ATOM | 4474 | CB | LEU | C | 38 | 53.140 | 47.752 | 9.480 | 1.00 | 64.47 | C |
| ATOM | 4475 | CG | LEU | C | 38 | 52.074 | 48.539 | 10.239 | 1.00 | 74.81 | C |
| ATOM | 4476 | CD1 | LEU | C | 38 | 51.615 | 47.749 | 11.450 | 1.00 | 59.27 | C |
| ATOM | 4477 | CD2 | LEU | C | 38 | 52.612 | 49.888 | 10.653 | 1.00 | 75.98 | C |
| ATOM | 4478 | N | LEU | C | 39 | 53.049 | 47.470 | 6.431 | 1.00 | 54.36 | N |
| ATOM | 4479 | CA | LEU | C | 39 | 52.690 | 47.874 | 5.071 | 1.00 | 72.39 | C |
| ATOM | 4480 | C | LEU | C | 39 | 52.065 | 46.714 | 4.309 | 1.00 | 62.03 | C |
| ATOM | 4481 | O | LEU | C | 39 | 51.230 | 46.909 | 3.426 | 1.00 | 62.94 | O |
| ATOM | 4482 | CB | LEU | C | 39 | 53.887 | 48.439 | 4.286 | 1.00 | 78.71 | C |
| ATOM | 4483 | CG | LEU | C | 39 | 54.230 | 49.934 | 4.429 | 1.00 | 96.84 | C |
| ATOM | 4484 | CD1 | LEU | C | 39 | 54.644 | 50.534 | 3.079 | 1.00 | 76.04 | C |
| ATOM | 4485 | CD2 | LEU | C | 39 | 53.074 | 50.742 | 5.028 | 1.00 | 76.37 | C |
| ATOM | 4486 | N | MET | C | 40 | 52.470 | 45.501 | 4.654 | 1.00 | 61.96 | N |
| ATOM | 4487 | CA | MET | C | 40 | 51.911 | 44.332 | 4.003 | 1.00 | 56.57 | C |
| ATOM | 4488 | C | MET | C | 40 | 50.470 | 44.158 | 4.473 | 1.00 | 47.73 | C |
| ATOM | 4489 | O | MET | C | 40 | 49.551 | 44.058 | 3.664 | 1.00 | 47.30 | O |
| ATOM | 4490 | CB | MET | C | 40 | 52.758 | 43.093 | 4.299 | 1.00 | 45.62 | C |
| ATOM | 4491 | CG | MET | C | 40 | 53.064 | 42.248 | 3.063 | 1.00 | 52.12 | C |
| ATOM | 4492 | SD | MET | C | 40 | 54.007 | 43.090 | 1.764 | 1.00 | 77.40 | S |
| ATOM | 4493 | CE | MET | C | 40 | 55.643 | 43.128 | 2.494 | 1.00 | 75.54 | C |
| ATOM | 4494 | N | ALA | C | 41 | 50.275 | 44.153 | 5.785 | 1.00 | 41.08 | N |
| ATOM | 4495 | CA | ALA | C | 41 | 48.939 | 44.066 | 6.345 | 1.00 | 41.50 | C |
| ATOM | 4496 | C | ALA | C | 41 | 48.034 | 45.152 | 5.756 | 1.00 | 46.54 | C |
| ATOM | 4497 | O | ALA | C | 41 | 46.817 | 44.976 | 5.657 | 1.00 | 36.41 | O |
| ATOM | 4498 | CB | ALA | C | 41 | 49.004 | 44.180 | 7.846 | 1.00 | 34.26 | C |
| ATOM | 4499 | N | LEU | C | 42 | 48.641 | 46.265 | 5.358 | 1.00 | 48.53 | N |
| ATOM | 4500 | CA | LEU | C | 42 | 47.901 | 47.400 | 4.818 | 1.00 | 51.39 | C |
| ATOM | 4501 | C | LEU | C | 42 | 47.378 | 47.125 | 3.413 | 1.00 | 52.74 | C |
| ATOM | 4502 | O | LEU | C | 42 | 46.209 | 47.380 | 3.121 | 1.00 | 52.16 | O |
| ATOM | 4503 | CB | LEU | C | 42 | 48.782 | 48.647 | 4.781 | 1.00 | 63.53 | C |
| ATOM | 4504 | CG | LEU | C | 42 | 48.034 | 49.975 | 4.871 | 1.00 | 68.94 | C |
| ATOM | 4505 | CD1 | LEU | C | 42 | 47.935 | 50.383 | 6.336 | 1.00 | 50.82 | C |
| ATOM | 4506 | CD2 | LEU | C | 42 | 48.727 | 51.051 | 4.049 | 1.00 | 59.08 | C |
| ATOM | 4507 | N | VAL | C | 43 | 48.248 | 46.618 | 2.542 | 1.00 | 47.79 | N |
| ATOM | 4508 | CA | VAL | C | 43 | 47.838 | 46.288 | 1.181 | 1.00 | 43.61 | C |
| ATOM | 4509 | C | VAL | C | 43 | 46.746 | 45.229 | 1.195 | 1.00 | 41.83 | C |
| ATOM | 4510 | O | VAL | C | 43 | 45.739 | 45.369 | 0.506 | 1.00 | 48.48 | O |
| ATOM | 4511 | CB | VAL | C | 43 | 49.019 | 45.830 | 0.286 | 1.00 | 41.39 | C |
| ATOM | 4512 | CG1 | VAL | C | 43 | 50.086 | 46.897 | 0.232 | 1.00 | 44.46 | C |
| ATOM | 4513 | CG2 | VAL | C | 43 | 49.606 | 44.536 | 0.789 | 1.00 | 47.99 | C |
| ATOM | 4514 | N | VAL | C | 44 | 46.932 | 44.179 | 1.988 | 1.00 | 33.84 | N |
| ATOM | 4515 | CA | VAL | C | 44 | 45.909 | 43.149 | 2.104 | 1.00 | 34.68 | C |
| ATOM | 4516 | C | VAL | C | 44 | 44.562 | 43.792 | 2.440 | 1.00 | 43.41 | C |
| ATOM | 4517 | O | VAL | C | 44 | 43.510 | 43.337 | 1.991 | 1.00 | 38.69 | O |
| ATOM | 4518 | CB | VAL | C | 44 | 46.277 | 42.111 | 3.170 | 1.00 | 27.15 | C |
| ATOM | 4519 | CG1 | VAL | C | 44 | 45.091 | 41.206 | 3.467 | 1.00 | 21.09 | C |
| ATOM | 4520 | CG2 | VAL | C | 44 | 47.479 | 41.304 | 2.732 | 1.00 | 31.64 | C |
| ATOM | 4521 | N | LEU | C | 45 | 44.614 | 44.871 | 3.217 | 1.00 | 48.46 | N |
| ATOM | 4522 | CA | LEU | C | 45 | 43.413 | 45.596 | 3.621 | 1.00 | 52.10 | C |
| ATOM | 4523 | C | LEU | C | 45 | 42.768 | 46.351 | 2.460 | 1.00 | 44.83 | C |
| ATOM | 4524 | O | LEU | C | 45 | 41.555 | 46.287 | 2.267 | 1.00 | 40.42 | O |
| ATOM | 4525 | CB | LEU | C | 45 | 43.739 | 46.579 | 4.745 | 1.00 | 57.12 | C |
| ATOM | 4526 | CG | LEU | C | 45 | 42.544 | 47.444 | 5.140 | 1.00 | 56.39 | C |
| ATOM | 4527 | CD1 | LEU | C | 45 | 41.495 | 46.578 | 5.810 | 1.00 | 48.18 | C |
| ATOM | 4528 | CD2 | LEU | C | 45 | 42.972 | 48.591 | 6.037 | 1.00 | 44.29 | C |
| ATOM | 4529 | N | LEU | C | 46 | 43.585 | 47.093 | 1.716 | 1.00 | 41.67 | N |
| ATOM | 4530 | CA | LEU | C | 46 | 43.137 | 47.744 | 0.493 | 1.00 | 42.00 | C |
| ATOM | 4531 | C | LEU | C | 46 | 42.459 | 46.739 | −0.430 | 1.00 | 49.10 | C |
| ATOM | 4532 | O | LEU | C | 46 | 41.241 | 46.775 | −0.609 | 1.00 | 50.26 | O |
| ATOM | 4533 | CB | LEU | C | 46 | 44.323 | 48.369 | −0.233 | 1.00 | 45.05 | C |
| ATOM | 4534 | CG | LEU | C | 46 | 44.596 | 49.837 | 0.060 | 1.00 | 48.17 | C |
| ATOM | 4535 | CD1 | LEU | C | 46 | 45.698 | 50.364 | −0.852 | 1.00 | 44.38 | C |
| ATOM | 4536 | CD2 | LEU | C | 46 | 43.314 | 50.621 | −0.140 | 1.00 | 55.66 | C |
| ATOM | 4537 | N | ILE | C | 47 | 43.262 | 45.843 | −1.005 | 1.00 | 43.20 | N |
| ATOM | 4538 | CA | ILE | C | 47 | 42.770 | 44.787 | −1.884 | 1.00 | 48.27 | C |
| ATOM | 4539 | C | ILE | C | 47 | 41.515 | 44.100 | −1.356 | 1.00 | 40.95 | C |
| ATOM | 4540 | O | ILE | C | 47 | 40.548 | 43.928 | −2.090 | 1.00 | 44.08 | O |
| ATOM | 4541 | CB | ILE | C | 47 | 43.835 | 43.693 | −2.128 | 1.00 | 48.44 | C |
| ATOM | 4542 | CG1 | ILE | C | 47 | 45.103 | 44.298 | −2.727 | 1.00 | 41.36 | C |
| ATOM | 4543 | CG2 | ILE | C | 47 | 43.285 | 42.594 | −3.038 | 1.00 | 34.31 | C |
| ATOM | 4544 | CD1 | ILE | C | 47 | 46.295 | 43.371 | −2.650 | 1.00 | 37.34 | C |
| ATOM | 4545 | N | VAL | C | 48 | 41.523 | 43.687 | −0.097 | 1.00 | 32.06 | N |
| ATOM | 4546 | CA | VAL | C | 48 | 40.371 | 42.950 | 0.398 | 1.00 | 38.94 | C |
| ATOM | 4547 | C | VAL | C | 48 | 39.137 | 43.828 | 0.573 | 1.00 | 46.74 | C |
| ATOM | 4548 | O | VAL | C | 48 | 38.031 | 43.430 | 0.201 | 1.00 | 52.34 | O |
| ATOM | 4549 | CB | VAL | C | 48 | 40.645 | 42.183 | 1.695 | 1.00 | 34.72 | C |
| ATOM | 4550 | CG1 | VAL | C | 48 | 39.333 | 41.632 | 2.235 | 1.00 | 31.10 | C |
| ATOM | 4551 | CG2 | VAL | C | 48 | 41.630 | 41.046 | 1.442 | 1.00 | 31.62 | C |
| ATOM | 4552 | N | ALA | C | 49 | 39.317 | 45.022 | 1.127 | 1.00 | 48.15 | N |
| ATOM | 4553 | CA | ALA | C | 49 | 38.181 | 45.913 | 1.353 | 1.00 | 49.66 | C |
| ATOM | 4554 | C | ALA | C | 49 | 37.560 | 46.410 | 0.039 | 1.00 | 50.65 | C |
| ATOM | 4555 | O | ALA | C | 49 | 36.343 | 46.339 | −0.162 | 1.00 | 42.51 | O |
| ATOM | 4556 | CB | ALA | C | 49 | 38.585 | 47.082 | 2.232 | 1.00 | 32.74 | C |
| ATOM | 4557 | N | GLY | C | 50 | 38.402 | 46.910 | −0.855 | 1.00 | 44.28 | N |
| ATOM | 4558 | CA | GLY | C | 50 | 37.925 | 47.496 | −2.092 | 1.00 | 50.62 | C |
| ATOM | 4559 | C | GLY | C | 50 | 37.223 | 46.512 | −3.005 | 1.00 | 49.92 | C |
| ATOM | 4560 | O | GLY | C | 50 | 36.278 | 46.863 | −3.709 | 1.00 | 54.95 | O |
| ATOM | 4561 | N | ASN | C | 51 | 37.687 | 45.272 | −3.006 | 1.00 | 45.78 | N |
| ATOM | 4562 | CA | ASN | C | 51 | 37.132 | 44.283 | −3.908 | 1.00 | 42.07 | C |
| ATOM | 4563 | C | ASN | C | 51 | 35.901 | 43.627 | −3.330 | 1.00 | 45.47 | C |
| ATOM | 4564 | O | ASN | C | 51 | 35.089 | 43.076 | −4.063 | 1.00 | 54.88 | O |
| ATOM | 4565 | CB | ASN | C | 51 | 38.178 | 43.235 | −4.265 | 1.00 | 44.54 | C |
| ATOM | 4566 | CG | ASN | C | 51 | 39.097 | 43.696 | −5.368 | 1.00 | 50.86 | C |
| ATOM | 4567 | OD1 | ASN | C | 51 | 38.698 | 43.755 | −6.533 | 1.00 | 48.17 | O |
| ATOM | 4568 | ND2 | ASN | C | 51 | 40.340 | 44.033 | −5.011 | 1.00 | 47.17 | N |
| ATOM | 4569 | N | VAL | C | 52 | 35.768 | 43.677 | −2.012 | 1.00 | 39.71 | N |
| ATOM | 4570 | CA | VAL | C | 52 | 34.572 | 43.164 | −1.372 | 1.00 | 45.09 | C |
| ATOM | 4571 | C | VAL | C | 52 | 33.490 | 44.220 | −1.533 | 1.00 | 46.91 | C |
| ATOM | 4572 | O | VAL | C | 52 | 32.300 | 43.912 | −1.609 | 1.00 | 42.98 | O |
| ATOM | 4573 | CB | VAL | C | 52 | 34.829 | 42.824 | 0.117 | 1.00 | 41.79 | C |
| ATOM | 4574 | CG1 | VAL | C | 52 | 33.526 | 42.705 | 0.889 | 1.00 | 21.89 | C |
| ATOM | 4575 | CG2 | VAL | C | 52 | 35.624 | 41.538 | 0.231 | 1.00 | 37.43 | C |
| ATOM | 4576 | N | LEU | C | 53 | 33.937 | 45.470 | −1.611 | 1.00 | 50.38 | N |
| ATOM | 4577 | CA | LEU | C | 53 | 33.069 | 46.623 | −1.821 | 1.00 | 51.68 | C |
| ATOM | 4578 | C | LEU | C | 53 | 32.412 | 46.549 | −3.192 | 1.00 | 53.17 | C |
| ATOM | 4579 | O | LEU | C | 53 | 31.200 | 46.699 | −3.324 | 1.00 | 53.58 | O |
| ATOM | 4580 | CB | LEU | C | 53 | 33.899 | 47.901 | −1.728 | 1.00 | 59.29 | C |
| ATOM | 4581 | CG | LEU | C | 53 | 33.299 | 49.057 | −0.935 | 1.00 | 69.13 | C |
| ATOM | 4582 | CD1 | LEU | C | 53 | 32.797 | 48.562 | 0.414 | 1.00 | 52.50 | C |
| ATOM | 4583 | CD2 | LEU | C | 53 | 34.342 | 50.143 | −0.754 | 1.00 | 78.61 | C |
| ATOM | 4584 | N | VAL | C | 54 | 33.233 | 46.319 | −4.210 | 1.00 | 54.77 | N |
| ATOM | 4585 | CA | VAL | C | 54 | 32.756 | 46.140 | −5.571 | 1.00 | 48.50 | C |
| ATOM | 4586 | C | VAL | C | 54 | 31.727 | 45.015 | −5.661 | 1.00 | 50.61 | C |
| ATOM | 4587 | O | VAL | C | 54 | 30.598 | 45.234 | −6.084 | 1.00 | 52.82 | O |
| ATOM | 4588 | CB | VAL | C | 54 | 33.920 | 45.844 | −6.522 | 1.00 | 45.32 | C |
| ATOM | 4589 | CG1 | VAL | C | 54 | 33.403 | 45.432 | −7.895 | 1.00 | 50.00 | C |
| ATOM | 4590 | CG2 | VAL | C | 54 | 34.835 | 47.055 | −6.621 | 1.00 | 45.78 | C |
| ATOM | 4591 | N | ILE | C | 55 | 32.119 | 43.814 | −5.255 | 1.00 | 47.56 | N |
| ATOM | 4592 | CA | ILE | C | 55 | 31.236 | 42.657 | −5.337 | 1.00 | 49.13 | C |
| ATOM | 4593 | C | ILE | C | 55 | 29.918 | 42.898 | −4.618 | 1.00 | 54.45 | C |
| ATOM | 4594 | O | ILE | C | 55 | 28.860 | 42.471 | −5.082 | 1.00 | 65.43 | O |
| ATOM | 4595 | CB | ILE | C | 55 | 31.889 | 41.392 | −4.760 | 1.00 | 48.28 | C |
| ATOM | 4596 | CG1 | ILE | C | 55 | 33.024 | 40.910 | −5.671 | 1.00 | 44.06 | C |
| ATOM | 4597 | CG2 | ILE | C | 55 | 30.854 | 40.299 | −4.584 | 1.00 | 36.19 | C |
| ATOM | 4598 | CD1 | ILE | C | 55 | 34.054 | 40.027 | −4.964 | 1.00 | 37.14 | C |
| ATOM | 4599 | N | ALA | C | 56 | 29.973 | 43.587 | −3.487 | 1.00 | 54.42 | N |
| ATOM | 4600 | CA | ALA | C | 56 | 28.755 | 43.862 | −2.727 | 1.00 | 54.56 | C |
| ATOM | 4601 | C | ALA | C | 56 | 27.909 | 44.959 | −3.369 | 1.00 | 56.07 | C |
| ATOM | 4602 | O | ALA | C | 56 | 26.687 | 44.858 | −3.409 | 1.00 | 58.96 | O |
| ATOM | 4603 | CB | ALA | C | 56 | 29.090 | 44.216 | −1.300 | 1.00 | 40.87 | C |
| ATOM | 4604 | N | ALA | C | 57 | 28.561 | 46.002 | −3.876 | 1.00 | 54.31 | N |
| ATOM | 4605 | CA | ALA | C | 57 | 27.854 | 47.107 | −4.522 | 1.00 | 60.09 | C |
| ATOM | 4606 | C | ALA | C | 57 | 27.045 | 46.625 | −5.721 | 1.00 | 72.86 | C |
| ATOM | 4607 | O | ALA | C | 57 | 25.888 | 47.008 | −5.898 | 1.00 | 77.43 | O |
| ATOM | 4608 | CB | ALA | C | 57 | 28.832 | 48.188 | −4.950 | 1.00 | 57.46 | C |
| ATOM | 4609 | N | ILE | C | 58 | 27.668 | 45.790 | −6.546 | 1.00 | 72.60 | N |
| ATOM | 4610 | CA | ILE | C | 58 | 27.006 | 45.216 | −7.707 | 1.00 | 68.68 | C |
| ATOM | 4611 | C | ILE | C | 58 | 25.900 | 44.279 | −7.243 | 1.00 | 65.99 | C |
| ATOM | 4612 | O | ILE | C | 58 | 24.821 | 44.236 | −7.827 | 1.00 | 77.27 | O |
| ATOM | 4613 | CB | ILE | C | 58 | 28.012 | 44.478 | −8.617 | 1.00 | 54.52 | C |
| ATOM | 4614 | CG1 | ILE | C | 58 | 28.961 | 45.483 | −9.260 | 1.00 | 46.71 | C |
| ATOM | 4615 | CG2 | ILE | C | 58 | 27.298 | 43.684 | −9.700 | 1.00 | 47.13 | C |
| ATOM | 4616 | CD1 | ILE | C | 58 | 30.109 | 44.845 | −10.001 | 1.00 | 54.09 | C |
| ATOM | 4617 | N | GLY | C | 59 | 26.169 | 43.545 | −6.172 | 1.00 | 64.64 | N |
| ATOM | 4618 | CA | GLY | C | 59 | 25.177 | 42.655 | −5.599 | 1.00 | 82.78 | C |
| ATOM | 4619 | C | GLY | C | 59 | 23.985 | 43.392 | −5.011 | 1.00 | 90.05 | C |
| ATOM | 4620 | O | GLY | C | 59 | 22.879 | 42.851 | −4.951 | 1.00 | 83.53 | O |
| ATOM | 4621 | N | SER | C | 60 | 24.211 | 44.630 | −4.577 | 1.00 | 91.68 | N |
| ATOM | 4622 | CA | SER | C | 60 | 23.163 | 45.430 | −3.945 | 1.00 | 94.42 | C |
| ATOM | 4623 | C | SER | C | 60 | 22.216 | 46.046 | −4.970 | 1.00 | 88.91 | C |
| ATOM | 4624 | O | SER | C | 60 | 21.043 | 45.683 | −5.033 | 1.00 | 109.21 | O |
| ATOM | 4625 | CB | SER | C | 60 | 23.770 | 46.522 | −3.057 | 1.00 | 74.97 | C |
| ATOM | 4626 | OG | SER | C | 60 | 24.478 | 45.949 | −1.971 | 1.00 | 72.49 | O |
| ATOM | 4627 | N | THR | C | 61 | 22.727 | 46.975 | −5.768 | 1.00 | 82.88 | N |
| ATOM | 4628 | CA | THR | C | 61 | 21.926 | 47.618 | −6.804 | 1.00 | 99.39 | C |
| ATOM | 4629 | C | THR | C | 61 | 21.803 | 46.724 | −8.036 | 1.00 | 109.99 | C |
| ATOM | 4630 | O | THR | C | 61 | 22.806 | 46.204 | −8.531 | 1.00 | 109.28 | O |
| ATOM | 4631 | CB | THR | C | 61 | 22.562 | 48.949 | −7.250 | 1.00 | 107.69 | C |
| ATOM | 4632 | OG1 | THR | C | 61 | 23.376 | 49.477 | −6.193 | 1.00 | 104.62 | O |
| ATOM | 4633 | CG2 | THR | C | 61 | 21.486 | 49.963 | −7.638 | 1.00 | 103.40 | C |
| ATOM | 4634 | N | GLN | C | 62 | 20.582 | 46.543 | −8.534 | 1.00 | 106.69 | N |
| ATOM | 4635 | CA | GLN | C | 62 | 20.394 | 45.846 | −9.804 | 1.00 | 111.46 | C |
| ATOM | 4636 | C | GLN | C | 62 | 20.655 | 46.838 | −10.924 | 1.00 | 106.79 | C |
| ATOM | 4637 | O | GLN | C | 62 | 20.941 | 46.455 | −12.062 | 1.00 | 93.82 | O |
| ATOM | 4638 | CB | GLN | C | 62 | 18.979 | 45.301 | −9.933 | 1.00 | 117.58 | C |
| ATOM | 4639 | CG | GLN | C | 62 | 18.398 | 44.766 | −8.651 | 1.00 | 121.59 | C |
| ATOM | 4640 | CD | GLN | C | 62 | 16.909 | 45.011 | −8.577 | 1.00 | 102.55 | C |
| ATOM | 4641 | OE1 | GLN | C | 62 | 16.301 | 45.472 | −9.545 | 1.00 | 85.70 | O |
| ATOM | 4642 | NE2 | GLN | C | 62 | 16.312 | 44.714 | −7.426 | 1.00 | 96.22 | N |
| ATOM | 4643 | N | ARG | C | 63 | 20.533 | 48.120 | −10.593 | 1.00 | 100.21 | N |
| ATOM | 4644 | CA | ARG | C | 63 | 20.925 | 49.179 | −11.505 | 1.00 | 97.55 | C |
| ATOM | 4645 | C | ARG | C | 63 | 22.375 | 48.945 | −11.924 | 1.00 | 105.03 | C |
| ATOM | 4646 | O | ARG | C | 63 | 22.715 | 49.008 | −13.111 | 1.00 | 90.47 | O |
| ATOM | 4647 | CB | ARG | C | 63 | 20.788 | 50.540 | −10.830 | 1.00 | 85.16 | C |
| ATOM | 4648 | CG | ARG | C | 63 | 21.132 | 51.692 | −11.741 | 1.00 | 106.18 | C |
| ATOM | 4649 | CD | ARG | C | 63 | 21.392 | 52.963 | −10.969 | 1.00 | 113.67 | C |
| ATOM | 4650 | NE | ARG | C | 63 | 21.821 | 54.036 | −11.861 | 1.00 | 132.81 | N |
| ATOM | 4651 | CZ | ARG | C | 63 | 22.111 | 55.269 | −11.462 | 1.00 | 144.18 | C |
| ATOM | 4652 | NH1 | ARG | C | 63 | 22.020 | 55.593 | −10.180 | 1.00 | 147.80 | N |
| ATOM | 4653 | NH2 | ARG | C | 63 | 22.493 | 56.181 | −12.348 | 1.00 | 139.74 | N |
| ATOM | 4654 | N | LEU | C | 64 | 23.223 | 48.668 | −10.936 | 1.00 | 103.38 | N |
| ATOM | 4655 | CA | LEU | C | 64 | 24.613 | 48.303 | −11.188 | 1.00 | 95.78 | C |
| ATOM | 4656 | C | LEU | C | 64 | 24.749 | 46.897 | −11.793 | 1.00 | 90.56 | C |
| ATOM | 4657 | O | LEU | C | 64 | 25.809 | 46.533 | −12.295 | 1.00 | 77.59 | O |
| ATOM | 4658 | CB | LEU | C | 64 | 25.441 | 48.406 | −9.901 | 1.00 | 90.67 | C |
| ATOM | 4659 | CG | LEU | C | 64 | 26.122 | 49.737 | −9.569 | 1.00 | 91.86 | C |
| ATOM | 4660 | CD1 | LEU | C | 64 | 26.961 | 49.613 | −8.298 | 1.00 | 83.68 | C |
| ATOM | 4661 | CD2 | LEU | C | 64 | 26.987 | 50.201 | −10.730 | 1.00 | 70.29 | C |
| ATOM | 4662 | N | GLN | C | 65 | 23.685 | 46.101 | −11.743 | 1.00 | 87.87 | N |
| ATOM | 4663 | CA | GLN | C | 65 | 23.736 | 44.775 | −12.354 | 1.00 | 83.74 | C |
| ATOM | 4664 | C | GLN | C | 65 | 23.481 | 44.803 | −13.859 | 1.00 | 89.14 | C |
| ATOM | 4665 | O | GLN | C | 65 | 22.353 | 44.633 | −14.324 | 1.00 | 86.62 | O |
| ATOM | 4666 | CB | GLN | C | 65 | 22.797 | 43.791 | −11.659 | 1.00 | 94.05 | C |
| ATOM | 4667 | CG | GLN | C | 65 | 23.522 | 42.784 | −10.786 | 1.00 | 86.15 | C |
| ATOM | 4668 | CD | GLN | C | 65 | 22.573 | 41.876 | −10.033 | 1.00 | 100.19 | C |
| ATOM | 4669 | OE1 | GLN | C | 65 | 21.368 | 42.135 | −9.960 | 1.00 | 110.17 | O |
| ATOM | 4670 | NE2 | GLN | C | 65 | 23.113 | 40.804 | −9.464 | 1.00 | 84.23 | N |
| ATOM | 4671 | N | THR | C | 66 | 24.553 | 45.041 | −14.604 | 1.00 | 75.73 | N |
| ATOM | 4672 | CA | THR | C | 66 | 24.552 | 44.922 | −16.045 | 1.00 | 46.63 | C |
| ATOM | 4673 | C | THR | C | 66 | 25.315 | 43.647 | −16.346 | 1.00 | 54.00 | C |
| ATOM | 4674 | O | THR | C | 66 | 25.679 | 42.918 | −15.427 | 1.00 | 65.51 | O |
| ATOM | 4675 | CB | THR | C | 66 | 25.311 | 46.084 | −16.680 | 1.00 | 56.30 | C |
| ATOM | 4676 | OG1 | THR | C | 66 | 26.700 | 45.968 | −16.350 | 1.00 | 55.23 | O |
| ATOM | 4677 | CG2 | THR | C | 66 | 24.789 | 47.410 | −16.161 | 1.00 | 48.65 | C |
| ATOM | 4678 | N | LEU | C | 67 | 25.562 | 43.370 | −17.624 | 1.00 | 55.05 | N |
| ATOM | 4679 | CA | LEU | C | 67 | 26.374 | 42.217 | −18.001 | 1.00 | 45.27 | C |
| ATOM | 4680 | C | LEU | C | 67 | 27.832 | 42.534 | −17.754 | 1.00 | 52.34 | C |
| ATOM | 4681 | O | LEU | C | 67 | 28.567 | 41.729 | −17.179 | 1.00 | 52.53 | O |
| ATOM | 4682 | CB | LEU | C | 67 | 26.198 | 41.883 | −19.478 | 1.00 | 55.93 | C |
| ATOM | 4683 | CG | LEU | C | 67 | 25.033 | 40.995 | −19.888 | 1.00 | 48.38 | C |
| ATOM | 4684 | CD1 | LEU | C | 67 | 25.273 | 40.502 | −21.304 | 1.00 | 58.75 | C |
| ATOM | 4685 | CD2 | LEU | C | 67 | 24.899 | 39.833 | −18.917 | 1.00 | 43.87 | C |
| ATOM | 4686 | N | THR | C | 68 | 28.239 | 43.715 | −18.212 | 1.00 | 46.53 | N |
| ATOM | 4687 | CA | THR | C | 68 | 29.600 | 44.200 | −18.038 | 1.00 | 41.22 | C |
| ATOM | 4688 | C | THR | C | 68 | 30.063 | 44.013 | −16.603 | 1.00 | 45.68 | C |
| ATOM | 4689 | O | THR | C | 68 | 31.199 | 43.601 | −16.355 | 1.00 | 49.05 | O |
| ATOM | 4690 | CB | THR | C | 68 | 29.713 | 45.696 | −18.395 | 1.00 | 51.69 | C |
| ATOM | 4691 | OG1 | THR | C | 68 | 29.587 | 45.867 | −19.813 | 1.00 | 55.23 | O |
| ATOM | 4692 | CG2 | THR | C | 68 | 31.058 | 46.266 | −17.929 | 1.00 | 44.27 | C |
| ATOM | 4693 | N | ASN | C | 69 | 29.172 | 44.312 | −15.661 | 1.00 | 41.66 | N |
| ATOM | 4694 | CA | ASN | C | 69 | 29.505 | 44.249 | −14.250 | 1.00 | 41.97 | C |
| ATOM | 4695 | C | ASN | C | 69 | 29.567 | 42.828 | −13.724 | 1.00 | 40.00 | C |
| ATOM | 4696 | O | ASN | C | 69 | 30.156 | 42.568 | −12.673 | 1.00 | 39.29 | O |
| ATOM | 4697 | CB | ASN | C | 69 | 28.532 | 45.097 | −13.435 | 1.00 | 55.42 | C |
| ATOM | 4698 | CG | ASN | C | 69 | 28.748 | 46.575 | −13.650 | 1.00 | 48.66 | C |
| ATOM | 4699 | OD1 | ASN | C | 69 | 29.749 | 46.982 | −14.231 | 1.00 | 51.76 | O |
| ATOM | 4700 | ND2 | ASN | C | 69 | 27.811 | 47.385 | −13.192 | 1.00 | 60.90 | N |
| ATOM | 4701 | N | LEU | C | 70 | 28.961 | 41.909 | −14.464 | 1.00 | 37.66 | N |
| ATOM | 4702 | CA | LEU | C | 70 | 29.108 | 40.490 | −14.174 | 1.00 | 43.55 | C |
| ATOM | 4703 | C | LEU | C | 70 | 30.544 | 40.038 | −14.405 | 1.00 | 40.21 | C |
| ATOM | 4704 | O | LEU | C | 70 | 31.101 | 39.291 | −13.598 | 1.00 | 28.53 | O |
| ATOM | 4705 | CB | LEU | C | 70 | 28.149 | 39.669 | −15.029 | 1.00 | 51.13 | C |
| ATOM | 4706 | CG | LEU | C | 70 | 26.717 | 39.715 | −14.510 | 1.00 | 47.35 | C |
| ATOM | 4707 | CD1 | LEU | C | 70 | 25.864 | 38.659 | −15.206 | 1.00 | 44.90 | C |
| ATOM | 4708 | CD2 | LEU | C | 70 | 26.736 | 39.511 | −12.996 | 1.00 | 32.10 | C |
| ATOM | 4709 | N | PHE | C | 71 | 31.136 | 40.504 | −15.506 | 1.00 | 38.89 | N |
| ATOM | 4710 | CA | PHE | C | 71 | 32.535 | 40.214 | −15.804 | 1.00 | 41.67 | C |
| ATOM | 4711 | C | PHE | C | 71 | 33.476 | 40.943 | −14.853 | 1.00 | 36.73 | C |
| ATOM | 4712 | O | PHE | C | 71 | 34.544 | 40.433 | −14.515 | 1.00 | 33.70 | O |
| ATOM | 4713 | CB | PHE | C | 71 | 32.866 | 40.527 | −17.264 | 1.00 | 41.41 | C |
| ATOM | 4714 | CG | PHE | C | 71 | 32.045 | 39.738 | −18.241 | 1.00 | 39.93 | C |
| ATOM | 4715 | CD1 | PHE | C | 71 | 31.839 | 38.382 | −18.046 | 1.00 | 38.30 | C |
| ATOM | 4716 | CD2 | PHE | C | 71 | 31.476 | 40.350 | −19.352 | 1.00 | 43.04 | C |
| ATOM | 4717 | CE1 | PHE | C | 71 | 31.069 | 37.646 | −18.929 | 1.00 | 44.22 | C |
| ATOM | 4718 | CE2 | PHE | C | 71 | 30.708 | 39.627 | −20.240 | 1.00 | 36.61 | C |
| ATOM | 4719 | CZ | PHE | C | 71 | 30.502 | 38.268 | −20.028 | 1.00 | 41.31 | C |
| ATOM | 4720 | N | ILE | C | 72 | 33.058 | 42.128 | −14.415 | 1.00 | 40.69 | N |
| ATOM | 4721 | CA | ILE | C | 72 | 33.784 | 42.904 | −13.411 | 1.00 | 39.23 | C |
| ATOM | 4722 | C | ILE | C | 72 | 33.826 | 42.188 | −12.062 | 1.00 | 33.50 | C |
| ATOM | 4723 | O | ILE | C | 72 | 34.815 | 42.259 | −11.335 | 1.00 | 33.81 | O |
| ATOM | 4724 | CB | ILE | C | 72 | 33.136 | 44.278 | −13.217 | 1.00 | 39.52 | C |
| ATOM | 4725 | CG1 | ILE | C | 72 | 33.377 | 45.151 | −14.446 | 1.00 | 40.15 | C |
| ATOM | 4726 | CG2 | ILE | C | 72 | 33.673 | 44.955 | −11.980 | 1.00 | 38.97 | C |
| ATOM | 4727 | CD1 | ILE | C | 72 | 34.695 | 45.813 | −14.452 | 1.00 | 39.12 | C |
| ATOM | 4728 | N | THR | C | 73 | 32.745 | 41.503 | −11.723 | 1.00 | 34.11 | N |
| ATOM | 4729 | CA | THR | C | 73 | 32.714 | 40.734 | −10.489 | 1.00 | 34.76 | C |
| ATOM | 4730 | C | THR | C | 73 | 33.728 | 39.607 | −10.567 | 1.00 | 36.07 | C |
| ATOM | 4731 | O | THR | C | 73 | 34.427 | 39.313 | −9.594 | 1.00 | 30.89 | O |
| ATOM | 4732 | CB | THR | C | 73 | 31.317 | 40.134 | −10.225 | 1.00 | 41.43 | C |
| ATOM | 4733 | OG1 | THR | C | 73 | 30.368 | 41.189 | −10.007 | 1.00 | 38.58 | O |
| ATOM | 4734 | CG2 | THR | C | 73 | 31.356 | 39.233 | −9.004 | 1.00 | 36.46 | C |
| ATOM | 4735 | N | SER | C | 74 | 33.794 | 38.976 | −11.737 | 1.00 | 37.38 | N |
| ATOM | 4736 | CA | SER | C | 74 | 34.726 | 37.882 | −11.972 | 1.00 | 36.06 | C |
| ATOM | 4737 | C | SER | C | 74 | 36.137 | 38.385 | −11.714 | 1.00 | 34.33 | C |
| ATOM | 4738 | O | SER | C | 74 | 36.956 | 37.718 | −11.076 | 1.00 | 30.56 | O |
| ATOM | 4739 | CB | SER | C | 74 | 34.588 | 37.377 | −13.406 | 1.00 | 31.77 | C |
| ATOM | 4740 | OG | SER | C | 74 | 35.537 | 36.363 | −13.675 | 1.00 | 42.61 | O |
| ATOM | 4741 | N | LEU | C | 75 | 36.394 | 39.585 | −12.213 | 1.00 | 32.95 | N |
| ATOM | 4742 | CA | LEU | C | 75 | 37.642 | 40.284 | −11.988 | 1.00 | 29.89 | C |
| ATOM | 4743 | C | LEU | C | 75 | 37.867 | 40.526 | −10.493 | 1.00 | 36.80 | C |
| ATOM | 4744 | O | LEU | C | 75 | 38.954 | 40.263 | −9.975 | 1.00 | 40.37 | O |
| ATOM | 4745 | CB | LEU | C | 75 | 37.606 | 41.610 | −12.734 | 1.00 | 36.02 | C |
| ATOM | 4746 | CG | LEU | C | 75 | 38.870 | 42.037 | −13.460 | 1.00 | 34.26 | C |
| ATOM | 4747 | CD1 | LEU | C | 75 | 39.428 | 40.859 | −14.215 | 1.00 | 38.09 | C |
| ATOM | 4748 | CD2 | LEU | C | 75 | 38.535 | 43.187 | −14.396 | 1.00 | 34.71 | C |
| ATOM | 4749 | N | ALA | C | 76 | 36.837 | 41.019 | −9.802 | 1.00 | 32.29 | N |
| ATOM | 4750 | CA | ALA | C | 76 | 36.919 | 41.263 | −8.362 | 1.00 | 33.46 | C |
| ATOM | 4751 | C | ALA | C | 76 | 37.298 | 40.005 | −7.573 | 1.00 | 36.45 | C |
| ATOM | 4752 | O | ALA | C | 76 | 38.142 | 40.054 | −6.683 | 1.00 | 39.30 | O |
| ATOM | 4753 | CB | ALA | C | 76 | 35.617 | 41.838 | −7.843 | 1.00 | 32.92 | C |
| ATOM | 4754 | N | CYS | C | 77 | 36.665 | 38.883 | −7.896 | 1.00 | 37.86 | N |
| ATOM | 4755 | CA | CYS | C | 77 | 36.959 | 37.624 | −7.219 | 1.00 | 28.59 | C |
| ATOM | 4756 | C | CYS | C | 77 | 38.407 | 37.185 | −7.410 | 1.00 | 27.56 | C |
| ATOM | 4757 | O | CYS | C | 77 | 39.012 | 36.641 | −6.500 | 1.00 | 26.38 | O |
| ATOM | 4758 | CB | CYS | C | 77 | 36.020 | 36.519 | −7.695 | 1.00 | 22.07 | C |
| ATOM | 4759 | SG | CYS | C | 77 | 34.333 | 36.688 | −7.110 | 1.00 | 54.98 | S |
| ATOM | 4760 | N | ALA | C | 78 | 38.971 | 37.408 | −8.590 | 1.00 | 27.61 | N |
| ATOM | 4761 | CA | ALA | C | 78 | 40.354 | 37.019 | −8.799 | 1.00 | 30.16 | C |
| ATOM | 4762 | C | ALA | C | 78 | 41.286 | 37.923 | −8.002 | 1.00 | 34.87 | C |
| ATOM | 4763 | O | ALA | C | 78 | 42.349 | 37.493 | −7.573 | 1.00 | 42.66 | O |
| ATOM | 4764 | CB | ALA | C | 78 | 40.720 | 37.017 | −10.273 | 1.00 | 25.50 | C |
| ATOM | 4765 | N | ASP | C | 79 | 40.891 | 39.173 | −7.790 | 1.00 | 33.82 | N |
| ATOM | 4766 | CA | ASP | C | 79 | 41.682 | 40.063 | −6.945 | 1.00 | 31.82 | C |
| ATOM | 4767 | C | ASP | C | 79 | 41.534 | 39.691 | −5.459 | 1.00 | 38.56 | C |
| ATOM | 4768 | O | ASP | C | 79 | 42.473 | 39.863 | −4.666 | 1.00 | 36.01 | O |
| ATOM | 4769 | CB | ASP | C | 79 | 41.314 | 41.523 | −7.205 | 1.00 | 31.96 | C |
| ATOM | 4770 | CG | ASP | C | 79 | 41.646 | 41.967 | −8.631 | 1.00 | 56.44 | C |
| ATOM | 4771 | OD1 | ASP | C | 79 | 42.701 | 41.539 | −9.161 | 1.00 | 50.52 | O |
| ATOM | 4772 | OD2 | ASP | C | 79 | 40.856 | 42.749 | −9.221 | 1.00 | 60.23 | O |
| ATOM | 4773 | N | LEU | C | 80 | 40.367 | 39.156 | −5.097 | 1.00 | 24.84 | N |
| ATOM | 4774 | CA | LEU | C | 80 | 40.112 | 38.714 | −3.736 | 1.00 | 29.68 | C |
| ATOM | 4775 | C | LEU | C | 80 | 40.980 | 37.517 | −3.342 | 1.00 | 36.33 | C |
| ATOM | 4776 | O | LEU | C | 80 | 41.661 | 37.542 | −2.317 | 1.00 | 39.93 | O |
| ATOM | 4777 | CB | LEU | C | 80 | 38.636 | 38.373 | −3.554 | 1.00 | 42.03 | C |
| ATOM | 4778 | CG | LEU | C | 80 | 38.074 | 38.665 | −2.163 | 1.00 | 41.32 | C |
| ATOM | 4779 | CD1 | LEU | C | 80 | 38.262 | 40.137 | −1.806 | 1.00 | 37.47 | C |
| ATOM | 4780 | CD2 | LEU | C | 80 | 36.612 | 38.276 | −2.110 | 1.00 | 31.16 | C |
| ATOM | 4781 | N | VAL | C | 81 | 40.950 | 36.464 | −4.148 | 1.00 | 37.74 | N |
| ATOM | 4782 | CA | VAL | C | 81 | 41.862 | 35.345 | −3.946 | 1.00 | 40.81 | C |
| ATOM | 4783 | C | VAL | C | 81 | 43.286 | 35.851 | −3.674 | 1.00 | 32.99 | C |
| ATOM | 4784 | O | VAL | C | 81 | 43.947 | 35.371 | −2.763 | 1.00 | 30.15 | O |
| ATOM | 4785 | CB | VAL | C | 81 | 41.851 | 34.363 | −5.157 | 1.00 | 43.77 | C |
| ATOM | 4786 | CG1 | VAL | C | 81 | 42.794 | 33.194 | −4.913 | 1.00 | 30.80 | C |
| ATOM | 4787 | CG2 | VAL | C | 81 | 40.434 | 33.851 | −5.425 | 1.00 | 31.09 | C |
| ATOM | 4788 | N | VAL | C | 82 | 43.749 | 36.825 | −4.455 | 1.00 | 29.08 | N |
| ATOM | 4789 | CA | VAL | C | 82 | 45.098 | 37.374 | −4.275 | 1.00 | 37.68 | C |
| ATOM | 4790 | C | VAL | C | 82 | 45.284 | 38.080 | −2.926 | 1.00 | 38.64 | C |
| ATOM | 4791 | O | VAL | C | 82 | 46.281 | 37.869 | −2.236 | 1.00 | 33.33 | O |
| ATOM | 4792 | CB | VAL | C | 82 | 45.488 | 38.350 | −5.418 | 1.00 | 33.44 | C |
| ATOM | 4793 | CG1 | VAL | C | 82 | 46.799 | 39.073 | −5.091 | 1.00 | 28.86 | C |
| ATOM | 4794 | CG2 | VAL | C | 82 | 45.603 | 37.605 | −6.729 | 1.00 | 28.54 | C |
| ATOM | 4795 | N | GLY | C | 83 | 44.327 | 38.925 | −2.562 | 1.00 | 34.47 | N |
| ATOM | 4796 | CA | GLY | C | 83 | 44.392 | 39.619 | −1.292 | 1.00 | 41.60 | C |
| ATOM | 4797 | C | GLY | C | 83 | 44.293 | 38.679 | −0.102 | 1.00 | 33.76 | C |
| ATOM | 4798 | O | GLY | C | 83 | 44.853 | 38.944 | 0.956 | 1.00 | 26.96 | O |
| ATOM | 4799 | N | LEU | C | 84 | 43.570 | 37.580 | −0.284 | 1.00 | 34.34 | N |
| ATOM | 4800 | CA | LEU | C | 84 | 43.339 | 36.609 | 0.781 | 1.00 | 33.08 | C |
| ATOM | 4801 | C | LEU | C | 84 | 44.379 | 35.484 | 0.831 | 1.00 | 36.04 | C |
| ATOM | 4802 | O | LEU | C | 84 | 44.785 | 35.071 | 1.911 | 1.00 | 36.95 | O |
| ATOM | 4803 | CB | LEU | C | 84 | 41.945 | 35.992 | 0.645 | 1.00 | 35.32 | C |
| ATOM | 4804 | CG | LEU | C | 84 | 40.724 | 36.847 | 0.960 | 1.00 | 33.50 | C |
| ATOM | 4805 | CD1 | LEU | C | 84 | 39.481 | 35.978 | 0.889 | 1.00 | 23.24 | C |
| ATOM | 4806 | CD2 | LEU | C | 84 | 40.860 | 37.499 | 2.333 | 1.00 | 31.04 | C |
| ATOM | 4807 | N | LEU | C | 85 | 44.800 | 34.975 | −0.326 | 1.00 | 34.06 | N |
| ATOM | 4808 | CA | LEU | C | 85 | 45.745 | 33.856 | −0.352 | 1.00 | 29.53 | C |
| ATOM | 4809 | C | LEU | C | 85 | 47.128 | 34.196 | −0.907 | 1.00 | 31.87 | C |
| ATOM | 4810 | O | LEU | C | 85 | 48.138 | 33.958 | −0.249 | 1.00 | 38.80 | O |
| ATOM | 4811 | CB | LEU | C | 85 | 45.149 | 32.660 | −1.088 | 1.00 | 30.03 | C |
| ATOM | 4812 | CG | LEU | C | 85 | 43.919 | 32.116 | −0.369 | 1.00 | 33.08 | C |
| ATOM | 4813 | CD1 | LEU | C | 85 | 43.355 | 30.878 | −1.025 | 1.00 | 32.71 | C |
| ATOM | 4814 | CD2 | LEU | C | 85 | 44.284 | 31.819 | 1.060 | 1.00 | 39.71 | C |
| ATOM | 4815 | N | VAL | C | 86 | 47.183 | 34.757 | −2.105 | 1.00 | 30.65 | N |
| ATOM | 4816 | CA | VAL | C | 86 | 48.470 | 34.985 | −2.748 | 1.00 | 32.54 | C |
| ATOM | 4817 | C | VAL | C | 86 | 49.394 | 35.916 | −1.953 | 1.00 | 30.57 | C |
| ATOM | 4818 | O | VAL | C | 86 | 50.487 | 35.506 | −1.579 | 1.00 | 28.13 | O |
| ATOM | 4819 | CB | VAL | C | 86 | 48.311 | 35.474 | −4.192 | 1.00 | 25.61 | C |
| ATOM | 4820 | CG1 | VAL | C | 86 | 49.668 | 35.506 | −4.881 | 1.00 | 28.73 | C |
| ATOM | 4821 | CG2 | VAL | C | 86 | 47.351 | 34.573 | −4.925 | 1.00 | 21.50 | C |
| ATOM | 4822 | N | VAL | C | 87 | 48.952 | 37.146 | −1.678 | 1.00 | 35.92 | N |
| ATOM | 4823 | CA | VAL | C | 87 | 49.810 | 38.143 | −1.019 | 1.00 | 32.25 | C |
| ATOM | 4824 | C | VAL | C | 87 | 50.179 | 37.778 | 0.419 | 1.00 | 37.01 | C |
| ATOM | 4825 | O | VAL | C | 87 | 51.334 | 37.930 | 0.811 | 1.00 | 41.30 | O |
| ATOM | 4826 | CB | VAL | C | 87 | 49.211 | 39.570 | −1.054 | 1.00 | 34.14 | C |
| ATOM | 4827 | CG1 | VAL | C | 87 | 49.817 | 40.429 | 0.059 | 1.00 | 27.39 | C |
| ATOM | 4828 | CG2 | VAL | C | 87 | 49.425 | 40.211 | −2.422 | 1.00 | 28.56 | C |
| ATOM | 4829 | N | PRO | C | 88 | 49.201 | 37.313 | 1.216 | 1.00 | 34.86 | N |
| ATOM | 4830 | CA | PRO | C | 88 | 49.510 | 36.797 | 2.556 | 1.00 | 34.10 | C |
| ATOM | 4831 | C | PRO | C | 88 | 50.712 | 35.832 | 2.616 | 1.00 | 35.89 | C |
| ATOM | 4832 | O | PRO | C | 88 | 51.703 | 36.153 | 3.279 | 1.00 | 37.77 | O |
| ATOM | 4833 | CB | PRO | C | 88 | 48.204 | 36.104 | 2.961 | 1.00 | 26.29 | C |
| ATOM | 4834 | CG | PRO | C | 88 | 47.157 | 36.935 | 2.312 | 1.00 | 25.36 | C |
| ATOM | 4835 | CD | PRO | C | 88 | 47.746 | 37.441 | 1.008 | 1.00 | 33.04 | C |
| ATOM | 4836 | N | PHE | C | 89 | 50.639 | 34.679 | 1.956 | 1.00 | 27.82 | N |
| ATOM | 4837 | CA | PHE | C | 89 | 51.774 | 33.761 | 1.963 | 1.00 | 31.54 | C |
| ATOM | 4838 | C | PHE | C | 89 | 53.009 | 34.405 | 1.306 | 1.00 | 36.57 | C |
| ATOM | 4839 | O | PHE | C | 89 | 54.147 | 34.142 | 1.696 | 1.00 | 28.08 | O |
| ATOM | 4840 | CB | PHE | C | 89 | 51.457 | 32.500 | 1.177 | 1.00 | 30.17 | C |
| ATOM | 4841 | CG | PHE | C | 89 | 50.517 | 31.560 | 1.846 | 1.00 | 26.29 | C |
| ATOM | 4842 | CD1 | PHE | C | 89 | 50.986 | 30.382 | 2.410 | 1.00 | 30.97 | C |
| ATOM | 4843 | CD2 | PHE | C | 89 | 49.152 | 31.806 | 1.844 | 1.00 | 32.53 | C |
| ATOM | 4844 | CE1 | PHE | C | 89 | 50.116 | 29.468 | 2.992 | 1.00 | 33.06 | C |
| ATOM | 4845 | CE2 | PHE | C | 89 | 48.265 | 30.908 | 2.432 | 1.00 | 28.69 | C |
| ATOM | 4846 | CZ | PHE | C | 89 | 48.749 | 29.735 | 3.006 | 1.00 | 37.87 | C |
| ATOM | 4847 | N | GLY | C | 90 | 52.776 | 35.215 | 0.277 | 1.00 | 32.49 | N |
| ATOM | 4848 | CA | GLY | C | 90 | 53.859 | 35.851 | −0.452 | 1.00 | 29.73 | C |
| ATOM | 4849 | C | GLY | C | 90 | 54.607 | 36.855 | 0.403 | 1.00 | 35.22 | C |
| ATOM | 4850 | O | GLY | C | 90 | 55.810 | 37.064 | 0.246 | 1.00 | 24.87 | O |
| ATOM | 4851 | N | ALA | C | 91 | 53.879 | 37.480 | 1.321 | 1.00 | 40.02 | N |
| ATOM | 4852 | CA | ALA | C | 91 | 54.471 | 38.396 | 2.284 | 1.00 | 39.53 | C |
| ATOM | 4853 | C | ALA | C | 91 | 55.507 | 37.694 | 3.182 | 1.00 | 38.46 | C |
| ATOM | 4854 | O | ALA | C | 91 | 56.635 | 38.170 | 3.325 | 1.00 | 32.75 | O |
| ATOM | 4855 | CB | ALA | C | 91 | 53.387 | 39.023 | 3.115 | 1.00 | 33.11 | C |
| ATOM | 4856 | N | THR | C | 92 | 55.120 | 36.565 | 3.778 | 1.00 | 27.51 | N |
| ATOM | 4857 | CA | THR | C | 92 | 56.030 | 35.791 | 4.619 | 1.00 | 24.40 | C |
| ATOM | 4858 | C | THR | C | 92 | 57.316 | 35.479 | 3.874 | 1.00 | 30.89 | C |
| ATOM | 4859 | O | THR | C | 92 | 58.406 | 35.605 | 4.419 | 1.00 | 33.79 | O |
| ATOM | 4860 | CB | THR | C | 92 | 55.390 | 34.470 | 5.150 | 1.00 | 34.13 | C |
| ATOM | 4861 | OG1 | THR | C | 92 | 55.414 | 33.451 | 4.138 | 1.00 | 30.20 | O |
| ATOM | 4862 | CG2 | THR | C | 92 | 53.958 | 34.705 | 5.609 | 1.00 | 28.27 | C |
| ATOM | 4863 | N | LEU | C | 93 | 57.186 | 35.085 | 2.616 | 1.00 | 34.44 | N |
| ATOM | 4864 | CA | LEU | C | 93 | 58.345 | 34.768 | 1.798 | 1.00 | 36.58 | C |
| ATOM | 4865 | C | LEU | C | 93 | 59.289 | 35.959 | 1.632 | 1.00 | 35.22 | C |
| ATOM | 4866 | O | LEU | C | 93 | 60.508 | 35.791 | 1.577 | 1.00 | 39.31 | O |
| ATOM | 4867 | CB | LEU | C | 93 | 57.896 | 34.253 | 0.428 | 1.00 | 33.49 | C |
| ATOM | 4868 | CG | LEU | C | 93 | 58.936 | 33.740 | −0.567 | 1.00 | 25.62 | C |
| ATOM | 4869 | CD1 | LEU | C | 93 | 59.892 | 32.752 | 0.077 | 1.00 | 17.54 | C |
| ATOM | 4870 | CD2 | LEU | C | 93 | 58.187 | 33.083 | −1.707 | 1.00 | 31.92 | C |
| ATOM | 4871 | N | VAL | C | 94 | 58.734 | 37.160 | 1.539 | 1.00 | 34.62 | N |
| ATOM | 4872 | CA | VAL | C | 94 | 59.571 | 38.326 | 1.286 | 1.00 | 42.64 | C |
| ATOM | 4873 | C | VAL | C | 94 | 60.167 | 38.872 | 2.578 | 1.00 | 52.48 | C |
| ATOM | 4874 | O | VAL | C | 94 | 61.295 | 39.368 | 2.592 | 1.00 | 54.70 | O |
| ATOM | 4875 | CB | VAL | C | 94 | 58.810 | 39.437 | 0.546 | 1.00 | 45.58 | C |
| ATOM | 4876 | CG1 | VAL | C | 94 | 59.800 | 40.426 | −0.058 | 1.00 | 37.70 | C |
| ATOM | 4877 | CG2 | VAL | C | 94 | 57.938 | 38.837 | −0.544 | 1.00 | 51.12 | C |
| ATOM | 4878 | N | VAL | C | 95 | 59.411 | 38.772 | 3.663 | 1.00 | 40.50 | N |
| ATOM | 4879 | CA | VAL | C | 95 | 59.899 | 39.196 | 4.965 | 1.00 | 43.07 | C |
| ATOM | 4880 | C | VAL | C | 95 | 60.987 | 38.261 | 5.531 | 1.00 | 49.48 | C |
| ATOM | 4881 | O | VAL | C | 95 | 61.956 | 38.722 | 6.134 | 1.00 | 54.37 | O |
| ATOM | 4882 | CB | VAL | C | 95 | 58.741 | 39.334 | 5.974 | 1.00 | 40.04 | C |
| ATOM | 4883 | CG1 | VAL | C | 95 | 59.275 | 39.663 | 7.349 | 1.00 | 58.24 | C |
| ATOM | 4884 | CG2 | VAL | C | 95 | 57.765 | 40.404 | 5.513 | 1.00 | 51.46 | C |
| ATOM | 4885 | N | ARG | C | 96 | 60.837 | 36.955 | 5.329 | 1.00 | 42.34 | N |
| ATOM | 4886 | CA | ARG | C | 96 | 61.740 | 35.994 | 5.946 | 1.00 | 33.41 | C |
| ATOM | 4887 | C | ARG | C | 96 | 62.779 | 35.404 | 5.014 | 1.00 | 33.06 | C |
| ATOM | 4888 | O | ARG | C | 96 | 63.718 | 34.753 | 5.464 | 1.00 | 46.69 | O |
| ATOM | 4889 | CB | ARG | C | 96 | 60.952 | 34.865 | 6.610 | 1.00 | 34.33 | C |
| ATOM | 4890 | CG | ARG | C | 96 | 60.418 | 35.226 | 7.996 | 1.00 | 55.49 | C |
| ATOM | 4891 | CD | ARG | C | 96 | 61.555 | 35.460 | 9.000 | 1.00 | 45.07 | C |
| ATOM | 4892 | NE | ARG | C | 96 | 62.372 | 34.266 | 9.198 | 1.00 | 37.37 | N |
| ATOM | 4893 | CZ | ARG | C | 96 | 61.968 | 33.193 | 9.876 | 1.00 | 43.85 | C |
| ATOM | 4894 | NH1 | ARG | C | 96 | 60.754 | 33.167 | 10.419 | 1.00 | 28.51 | N |
| ATOM | 4895 | NH2 | ARG | C | 96 | 62.778 | 32.143 | 10.008 | 1.00 | 37.06 | N |
| ATOM | 4896 | N | GLY | C | 97 | 62.614 | 35.618 | 3.721 | 1.00 | 33.08 | N |
| ATOM | 4897 | CA | GLY | C | 97 | 63.492 | 35.001 | 2.739 | 1.00 | 27.90 | C |
| ATOM | 4898 | C | GLY | C | 97 | 63.513 | 33.477 | 2.778 | 1.00 | 32.65 | C |
| ATOM | 4899 | O | GLY | C | 97 | 64.519 | 32.866 | 2.436 | 1.00 | 45.61 | O |
| ATOM | 4900 | N | THR | C | 98 | 62.414 | 32.858 | 3.201 | 1.00 | 29.36 | N |
| ATOM | 4901 | CA | THR | C | 98 | 62.305 | 31.397 | 3.234 | 1.00 | 29.74 | C |
| ATOM | 4902 | C | THR | C | 98 | 60.838 | 31.043 | 3.157 | 1.00 | 35.04 | C |
| ATOM | 4903 | O | THR | C | 98 | 59.973 | 31.905 | 3.372 | 1.00 | 31.10 | O |
| ATOM | 4904 | CB | THR | C | 98 | 62.823 | 30.770 | 4.555 | 1.00 | 27.89 | C |
| ATOM | 4905 | OG1 | THR | C | 98 | 63.751 | 31.651 | 5.184 | 1.00 | 40.33 | O |
| ATOM | 4906 | CG2 | THR | C | 98 | 63.471 | 29.419 | 4.301 | 1.00 | 23.25 | C |
| ATOM | 4907 | N | TRP | C | 99 | 60.565 | 29.770 | 2.881 | 1.00 | 20.88 | N |
| ATOM | 4908 | CA | TRP | C | 99 | 59.207 | 29.275 | 2.856 | 1.00 | 19.61 | C |
| ATOM | 4909 | C | TRP | C | 99 | 58.918 | 28.485 | 4.128 | 1.00 | 24.95 | C |
| ATOM | 4910 | O | TRP | C | 99 | 59.577 | 27.484 | 4.411 | 1.00 | 24.39 | O |
| ATOM | 4911 | CB | TRP | C | 99 | 58.977 | 28.426 | 1.612 | 1.00 | 20.24 | C |
| ATOM | 4912 | CG | TRP | C | 99 | 57.543 | 28.104 | 1.366 | 1.00 | 22.90 | C |
| ATOM | 4913 | CD1 | TRP | C | 99 | 56.952 | 26.890 | 1.494 | 1.00 | 20.90 | C |
| ATOM | 4914 | CD2 | TRP | C | 99 | 56.511 | 29.016 | 0.963 | 1.00 | 19.43 | C |
| ATOM | 4915 | NE1 | TRP | C | 99 | 55.618 | 26.978 | 1.177 | 1.00 | 26.28 | N |
| ATOM | 4916 | CE2 | TRP | C | 99 | 55.322 | 28.274 | 0.847 | 1.00 | 23.08 | C |
| ATOM | 4917 | CE3 | TRP | C | 99 | 56.480 | 30.384 | 0.680 | 1.00 | 19.54 | C |
| ATOM | 4918 | CZ2 | TRP | C | 99 | 54.113 | 28.852 | 0.471 | 1.00 | 18.38 | C |
| ATOM | 4919 | CZ3 | TRP | C | 99 | 55.278 | 30.954 | 0.299 | 1.00 | 21.30 | C |
| ATOM | 4920 | CH2 | TRP | C | 99 | 54.112 | 30.188 | 0.203 | 1.00 | 21.48 | C |
| ATOM | 4921 | N | LEU | C | 100 | 57.932 | 28.942 | 4.897 | 1.00 | 24.19 | N |
| ATOM | 4922 | CA | LEU | C | 100 | 57.631 | 28.327 | 6.191 | 1.00 | 26.92 | C |
| ATOM | 4923 | C | LEU | C | 100 | 56.611 | 27.203 | 6.094 | 1.00 | 28.16 | C |
| ATOM | 4924 | O | LEU | C | 100 | 56.377 | 26.488 | 7.070 | 1.00 | 28.87 | O |
| ATOM | 4925 | CB | LEU | C | 100 | 57.082 | 29.354 | 7.196 | 1.00 | 28.77 | C |
| ATOM | 4926 | CG | LEU | C | 100 | 57.759 | 30.644 | 7.683 | 1.00 | 33.42 | C |
| ATOM | 4927 | CD1 | LEU | C | 100 | 57.096 | 31.022 | 8.992 | 1.00 | 32.17 | C |
| ATOM | 4928 | CD2 | LEU | C | 100 | 59.267 | 30.523 | 7.876 | 1.00 | 30.34 | C |
| ATOM | 4929 | N | TRP | C | 101 | 55.999 | 27.041 | 4.927 | 1.00 | 28.57 | N |
| ATOM | 4930 | CA | TRP | C | 101 | 54.730 | 26.323 | 4.871 | 1.00 | 24.65 | C |
| ATOM | 4931 | C | TRP | C | 101 | 54.765 | 24.930 | 4.233 | 1.00 | 25.38 | C |
| ATOM | 4932 | O | TRP | C | 101 | 53.751 | 24.247 | 4.198 | 1.00 | 32.27 | O |
| ATOM | 4933 | CB | TRP | C | 101 | 53.664 | 27.224 | 4.247 | 1.00 | 19.60 | C |
| ATOM | 4934 | CG | TRP | C | 101 | 53.709 | 28.615 | 4.819 | 1.00 | 24.28 | C |
| ATOM | 4935 | CD1 | TRP | C | 101 | 54.181 | 29.744 | 4.204 | 1.00 | 26.56 | C |
| ATOM | 4936 | CD2 | TRP | C | 101 | 53.311 | 29.021 | 6.135 | 1.00 | 25.10 | C |
| ATOM | 4937 | NE1 | TRP | C | 101 | 54.085 | 30.832 | 5.052 | 1.00 | 23.63 | N |
| ATOM | 4938 | CE2 | TRP | C | 101 | 53.553 | 30.413 | 6.241 | 1.00 | 25.25 | C |
| ATOM | 4939 | CE3 | TRP | C | 101 | 52.756 | 28.349 | 7.226 | 1.00 | 24.58 | C |
| ATOM | 4940 | CZ2 | TRP | C | 101 | 53.257 | 31.138 | 7.393 | 1.00 | 21.63 | C |
| ATOM | 4941 | CZ3 | TRP | C | 101 | 52.463 | 29.073 | 8.375 | 1.00 | 21.42 | C |
| ATOM | 4942 | CH2 | TRP | C | 101 | 52.718 | 30.453 | 8.446 | 1.00 | 23.63 | C |
| ATOM | 4943 | N | GLY | C | 102 | 55.925 | 24.497 | 3.751 | 1.00 | 30.89 | N |
| ATOM | 4944 | CA | GLY | C | 102 | 56.064 | 23.138 | 3.252 | 1.00 | 24.52 | C |
| ATOM | 4945 | C | GLY | C | 102 | 56.036 | 22.988 | 1.742 | 1.00 | 27.25 | C |
| ATOM | 4946 | O | GLY | C | 102 | 55.484 | 23.817 | 1.023 | 1.00 | 28.32 | O |
| ATOM | 4947 | N | SER | C | 103 | 56.626 | 21.908 | 1.250 | 1.00 | 27.87 | N |
| ATOM | 4948 | CA | SER | C | 103 | 56.721 | 21.706 | −0.193 | 1.00 | 23.71 | C |
| ATOM | 4949 | C | SER | C | 103 | 55.348 | 21.701 | −0.863 | 1.00 | 28.14 | C |
| ATOM | 4950 | O | SER | C | 103 | 55.159 | 22.326 | −1.901 | 1.00 | 28.86 | O |
| ATOM | 4951 | CB | SER | C | 103 | 57.488 | 20.422 | −0.523 | 1.00 | 23.74 | C |
| ATOM | 4952 | OG | SER | C | 103 | 58.771 | 20.426 | 0.088 | 1.00 | 41.82 | O |
| ATOM | 4953 | N | PHE | C | 104 | 54.382 | 21.007 | −0.273 | 1.00 | 24.10 | N |
| ATOM | 4954 | CA | PHE | C | 104 | 53.090 | 20.900 | −0.926 | 1.00 | 23.87 | C |
| ATOM | 4955 | C | PHE | C | 104 | 52.397 | 22.245 | −1.055 | 1.00 | 26.73 | C |
| ATOM | 4956 | O | PHE | C | 104 | 51.870 | 22.571 | −2.116 | 1.00 | 30.36 | O |
| ATOM | 4957 | CB | PHE | C | 104 | 52.170 | 19.910 | −0.226 | 1.00 | 26.53 | C |
| ATOM | 4958 | CG | PHE | C | 104 | 50.777 | 19.926 | −0.758 | 1.00 | 24.89 | C |
| ATOM | 4959 | CD1 | PHE | C | 104 | 50.519 | 19.500 | −2.054 | 1.00 | 27.85 | C |
| ATOM | 4960 | CD2 | PHE | C | 104 | 49.729 | 20.385 | 0.016 | 1.00 | 28.88 | C |
| ATOM | 4961 | CE1 | PHE | C | 104 | 49.241 | 19.511 | −2.563 | 1.00 | 20.54 | C |
| ATOM | 4962 | CE2 | PHE | C | 104 | 48.441 | 20.403 | −0.489 | 1.00 | 36.85 | C |
| ATOM | 4963 | CZ | PHE | C | 104 | 48.200 | 19.965 | −1.784 | 1.00 | 31.30 | C |
| ATOM | 4964 | N | LEU | C | 105 | 52.383 | 23.021 | 0.023 | 1.00 | 30.21 | N |
| ATOM | 4965 | CA | LEU | C | 105 | 51.724 | 24.323 | −0.019 | 1.00 | 24.99 | C |
| ATOM | 4966 | C | LEU | C | 105 | 52.474 | 25.273 | −0.931 | 1.00 | 25.78 | C |
| ATOM | 4967 | O | LEU | C | 105 | 51.877 | 26.213 | −1.463 | 1.00 | 22.68 | O |
| ATOM | 4968 | CB | LEU | C | 105 | 51.572 | 24.945 | 1.372 | 1.00 | 27.03 | C |
| ATOM | 4969 | CG | LEU | C | 105 | 50.452 | 24.467 | 2.310 | 1.00 | 30.97 | C |
| ATOM | 4970 | CD1 | LEU | C | 105 | 50.056 | 25.593 | 3.279 | 1.00 | 21.34 | C |
| ATOM | 4971 | CD2 | LEU | C | 105 | 49.248 | 23.993 | 1.527 | 1.00 | 27.82 | C |
| ATOM | 4972 | N | CYS | C | 106 | 53.777 | 25.031 | −1.116 | 1.00 | 24.65 | N |
| ATOM | 4973 | CA | CYS | C | 106 | 54.573 | 25.853 | −2.028 | 1.00 | 22.14 | C |
| ATOM | 4974 | C | CYS | C | 106 | 54.015 | 25.740 | −3.441 | 1.00 | 27.53 | C |
| ATOM | 4975 | O | CYS | C | 106 | 53.745 | 26.752 | −4.103 | 1.00 | 22.83 | O |
| ATOM | 4976 | CB | CYS | C | 106 | 56.039 | 25.438 | −2.019 | 1.00 | 23.24 | C |
| ATOM | 4977 | SG | CYS | C | 106 | 57.079 | 26.373 | −3.178 | 1.00 | 26.49 | S |
| ATOM | 4978 | N | GLU | C | 107 | 53.829 | 24.499 | −3.887 | 1.00 | 26.12 | N |
| ATOM | 4979 | CA | GLU | C | 107 | 53.316 | 24.234 | −5.222 | 1.00 | 22.41 | C |
| ATOM | 4980 | C | GLU | C | 107 | 51.913 | 24.753 | −5.362 | 1.00 | 23.24 | C |
| ATOM | 4981 | O | GLU | C | 107 | 51.614 | 25.458 | −6.315 | 1.00 | 22.18 | O |
| ATOM | 4982 | CB | GLU | C | 107 | 53.373 | 22.747 | −5.553 | 1.00 | 31.93 | C |
| ATOM | 4983 | CG | GLU | C | 107 | 54.483 | 22.401 | −6.531 | 1.00 | 52.80 | C |
| ATOM | 4984 | CD | GLU | C | 107 | 54.404 | 20.972 | −7.037 | 1.00 | 72.07 | C |
| ATOM | 4985 | OE1 | GLU | C | 107 | 54.906 | 20.708 | −8.164 | 1.00 | 61.95 | O |
| ATOM | 4986 | OE2 | GLU | C | 107 | 53.837 | 20.125 | −6.305 | 1.00 | 66.39 | O |
| ATOM | 4987 | N | LEU | C | 108 | 51.063 | 24.417 | −4.395 | 1.00 | 22.35 | N |
| ATOM | 4988 | CA | LEU | C | 108 | 49.693 | 24.909 | −4.364 | 1.00 | 25.20 | C |
| ATOM | 4989 | C | LEU | C | 108 | 49.607 | 26.431 | −4.464 | 1.00 | 26.48 | C |
| ATOM | 4990 | O | LEU | C | 108 | 48.812 | 26.965 | −5.244 | 1.00 | 28.90 | O |
| ATOM | 4991 | CB | LEU | C | 108 | 48.970 | 24.443 | −3.111 | 1.00 | 23.53 | C |
| ATOM | 4992 | CG | LEU | C | 108 | 47.459 | 24.629 | −3.237 | 1.00 | 23.55 | C |
| ATOM | 4993 | CD1 | LEU | C | 108 | 46.966 | 23.705 | −4.334 | 1.00 | 25.56 | C |
| ATOM | 4994 | CD2 | LEU | C | 108 | 46.745 | 24.349 | −1.911 | 1.00 | 22.23 | C |
| ATOM | 4995 | N | TRP | C | 109 | 50.420 | 27.120 | −3.670 | 1.00 | 25.27 | N |
| ATOM | 4996 | CA | TRP | C | 109 | 50.461 | 28.579 | −3.673 | 1.00 | 21.16 | C |
| ATOM | 4997 | C | TRP | C | 109 | 50.813 | 29.090 | −5.060 | 1.00 | 25.82 | C |
| ATOM | 4998 | O | TRP | C | 109 | 50.160 | 29.990 | −5.574 | 1.00 | 25.91 | O |
| ATOM | 4999 | CB | TRP | C | 109 | 51.480 | 29.084 | −2.637 | 1.00 | 21.91 | C |
| ATOM | 5000 | CG | TRP | C | 109 | 51.818 | 30.550 | −2.748 | 1.00 | 20.09 | C |
| ATOM | 5001 | CD1 | TRP | C | 109 | 50.986 | 31.596 | −2.516 | 1.00 | 23.45 | C |
| ATOM | 5002 | CD2 | TRP | C | 109 | 53.080 | 31.119 | −3.117 | 1.00 | 18.35 | C |
| ATOM | 5003 | NE1 | TRP | C | 109 | 51.641 | 32.783 | −2.720 | 1.00 | 19.96 | N |
| ATOM | 5004 | CE2 | TRP | C | 109 | 52.930 | 32.519 | −3.089 | 1.00 | 20.06 | C |
| ATOM | 5005 | CE3 | TRP | C | 109 | 54.325 | 30.580 | −3.462 | 1.00 | 23.18 | C |
| ATOM | 5006 | CZ2 | TRP | C | 109 | 53.976 | 33.393 | −3.401 | 1.00 | 22.55 | C |
| ATOM | 5007 | CZ3 | TRP | C | 109 | 55.363 | 31.448 | −3.773 | 1.00 | 24.24 | C |
| ATOM | 5008 | CH2 | TRP | C | 109 | 55.180 | 32.839 | −3.746 | 1.00 | 23.16 | C |
| ATOM | 5009 | N | THR | C | 110 | 51.856 | 28.508 | −5.653 | 1.00 | 26.48 | N |
| ATOM | 5010 | CA | THR | C | 110 | 52.337 | 28.898 | −6.979 | 1.00 | 23.25 | C |
| ATOM | 5011 | C | THR | C | 110 | 51.251 | 28.707 | −8.040 | 1.00 | 24.03 | C |
| ATOM | 5012 | O | THR | C | 110 | 50.966 | 29.623 | −8.802 | 1.00 | 25.42 | O |
| ATOM | 5013 | CB | THR | C | 110 | 53.614 | 28.111 | −7.355 | 1.00 | 24.26 | C |
| ATOM | 5014 | OG1 | THR | C | 110 | 54.665 | 28.456 | −6.449 | 1.00 | 28.58 | O |
| ATOM | 5015 | CG2 | THR | C | 110 | 54.068 | 28.416 | −8.777 | 1.00 | 16.00 | C |
| ATOM | 5016 | N | SER | C | 111 | 50.646 | 27.520 | −8.080 | 1.00 | 24.74 | N |
| ATOM | 5017 | CA | SER | C | 111 | 49.447 | 27.274 | −8.895 | 1.00 | 27.42 | C |
| ATOM | 5018 | C | SER | C | 111 | 48.401 | 28.400 | −8.818 | 1.00 | 30.00 | C |
| ATOM | 5019 | O | SER | C | 111 | 47.906 | 28.860 | −9.849 | 1.00 | 19.45 | O |
| ATOM | 5020 | CB | SER | C | 111 | 48.779 | 25.963 | −8.472 | 1.00 | 26.85 | C |
| ATOM | 5021 | OG | SER | C | 111 | 49.579 | 24.844 | −8.813 | 1.00 | 34.61 | O |
| ATOM | 5022 | N | LEU | C | 112 | 48.076 | 28.825 | −7.593 | 1.00 | 19.84 | N |
| ATOM | 5023 | CA | LEU | C | 112 | 47.065 | 29.853 | −7.337 | 1.00 | 20.69 | C |
| ATOM | 5024 | C | LEU | C | 112 | 47.414 | 31.214 | −7.888 | 1.00 | 24.15 | C |
| ATOM | 5025 | O | LEU | C | 112 | 46.567 | 31.937 | −8.410 | 1.00 | 25.01 | O |
| ATOM | 5026 | CB | LEU | C | 112 | 46.857 | 30.026 | −5.844 | 1.00 | 21.92 | C |
| ATOM | 5027 | CG | LEU | C | 112 | 45.726 | 29.181 | −5.303 | 1.00 | 33.90 | C |
| ATOM | 5028 | CD1 | LEU | C | 112 | 45.388 | 29.675 | −3.907 | 1.00 | 28.59 | C |
| ATOM | 5029 | CD2 | LEU | C | 112 | 44.534 | 29.276 | −6.261 | 1.00 | 22.69 | C |
| ATOM | 5030 | N | ASP | C | 113 | 48.673 | 31.568 | −7.725 | 1.00 | 20.05 | N |
| ATOM | 5031 | CA | ASP | C | 113 | 49.187 | 32.836 | −8.163 | 1.00 | 25.36 | C |
| ATOM | 5032 | C | ASP | C | 113 | 48.993 | 32.979 | −9.672 | 1.00 | 27.63 | C |
| ATOM | 5033 | O | ASP | C | 113 | 48.466 | 33.983 | −10.155 | 1.00 | 30.60 | O |
| ATOM | 5034 | CB | ASP | C | 113 | 50.667 | 32.862 | −7.807 | 1.00 | 30.35 | C |
| ATOM | 5035 | CG | ASP | C | 113 | 51.303 | 34.195 | −8.045 | 1.00 | 32.92 | C |
| ATOM | 5036 | OD1 | ASP | C | 113 | 50.765 | 34.997 | −8.840 | 1.00 | 33.21 | O |
| ATOM | 5037 | OD2 | ASP | C | 113 | 52.361 | 34.427 | −7.428 | 1.00 | 33.30 | O |
| ATOM | 5038 | N | VAL | C | 114 | 49.417 | 31.948 | −10.394 | 1.00 | 21.77 | N |
| ATOM | 5039 | CA | VAL | C | 114 | 49.391 | 31.903 | −11.844 | 1.00 | 23.57 | C |
| ATOM | 5040 | C | VAL | C | 114 | 47.965 | 31.859 | −12.395 | 1.00 | 26.37 | C |
| ATOM | 5041 | O | VAL | C | 114 | 47.630 | 32.558 | −13.365 | 1.00 | 23.16 | O |
| ATOM | 5042 | CB | VAL | C | 114 | 50.212 | 30.686 | −12.348 | 1.00 | 29.30 | C |
| ATOM | 5043 | CG1 | VAL | C | 114 | 50.087 | 30.508 | −13.839 | 1.00 | 24.31 | C |
| ATOM | 5044 | CG2 | VAL | C | 114 | 51.673 | 30.865 | −11.984 | 1.00 | 26.91 | C |
| ATOM | 5045 | N | LEU | C | 115 | 47.127 | 31.039 | −11.770 | 1.00 | 25.73 | N |
| ATOM | 5046 | CA | LEU | C | 115 | 45.706 | 30.987 | −12.100 | 1.00 | 18.78 | C |
| ATOM | 5047 | C | LEU | C | 115 | 45.057 | 32.366 | −12.050 | 1.00 | 22.53 | C |
| ATOM | 5048 | O | LEU | C | 115 | 44.332 | 32.749 | −12.971 | 1.00 | 24.52 | O |
| ATOM | 5049 | CB | LEU | C | 115 | 44.977 | 30.039 | −11.160 | 1.00 | 16.64 | C |
| ATOM | 5050 | CG | LEU | C | 115 | 43.489 | 29.887 | −11.435 | 1.00 | 23.31 | C |
| ATOM | 5051 | CD1 | LEU | C | 115 | 43.260 | 29.559 | −12.896 | 1.00 | 25.28 | C |
| ATOM | 5052 | CD2 | LEU | C | 115 | 42.917 | 28.808 | −10.557 | 1.00 | 21.26 | C |
| ATOM | 5053 | N | CYS | C | 116 | 45.328 | 33.118 | −10.984 | 1.00 | 24.03 | N |
| ATOM | 5054 | CA | CYS | C | 116 | 44.710 | 34.433 | −10.813 | 1.00 | 28.63 | C |
| ATOM | 5055 | C | CYS | C | 116 | 45.114 | 35.457 | −11.878 | 1.00 | 30.89 | C |
| ATOM | 5056 | O | CYS | C | 116 | 44.287 | 36.268 | −12.298 | 1.00 | 29.82 | O |
| ATOM | 5057 | CB | CYS | C | 116 | 44.930 | 34.982 | −9.406 | 1.00 | 19.38 | C |
| ATOM | 5058 | SG | CYS | C | 116 | 43.944 | 34.146 | −8.150 | 1.00 | 45.74 | S |
| ATOM | 5059 | N | VAL | C | 117 | 46.368 | 35.423 | −12.322 | 1.00 | 25.78 | N |
| ATOM | 5060 | CA | VAL | C | 117 | 46.810 | 36.353 | −13.365 | 1.00 | 29.93 | C |
| ATOM | 5061 | C | VAL | C | 117 | 46.317 | 35.921 | −14.752 | 1.00 | 31.70 | C |
| ATOM | 5062 | O | VAL | C | 117 | 45.958 | 36.759 | −15.574 | 1.00 | 22.36 | O |
| ATOM | 5063 | CB | VAL | C | 117 | 48.342 | 36.534 | −13.383 | 1.00 | 29.38 | C |
| ATOM | 5064 | CG1 | VAL | C | 117 | 48.722 | 37.707 | −14.283 | 1.00 | 30.30 | C |
| ATOM | 5065 | CG2 | VAL | C | 117 | 48.854 | 36.759 | −11.979 | 1.00 | 24.88 | C |
| ATOM | 5066 | N | THR | C | 118 | 46.288 | 34.614 | −15.008 | 1.00 | 32.57 | N |
| ATOM | 5067 | CA | THR | C | 118 | 45.751 | 34.112 | −16.270 | 1.00 | 27.99 | C |
| ATOM | 5068 | C | THR | C | 118 | 44.279 | 34.493 | −16.403 | 1.00 | 30.35 | C |
| ATOM | 5069 | O | THR | C | 118 | 43.859 | 35.008 | −17.453 | 1.00 | 22.53 | O |
| ATOM | 5070 | CB | THR | C | 118 | 45.887 | 32.579 | −16.401 | 1.00 | 30.37 | C |
| ATOM | 5071 | OG1 | THR | C | 118 | 47.271 | 32.208 | −16.363 | 1.00 | 27.91 | O |
| ATOM | 5072 | CG2 | THR | C | 118 | 45.276 | 32.093 | −17.706 | 1.00 | 22.03 | C |
| ATOM | 5073 | N | ALA | C | 119 | 43.503 | 34.248 | −15.343 | 1.00 | 20.70 | N |
| ATOM | 5074 | CA | ALA | C | 119 | 42.069 | 34.531 | −15.377 | 1.00 | 22.88 | C |
| ATOM | 5075 | C | ALA | C | 119 | 41.728 | 36.025 | −15.522 | 1.00 | 24.49 | C |
| ATOM | 5076 | O | ALA | C | 119 | 40.722 | 36.377 | −16.135 | 1.00 | 27.90 | O |
| ATOM | 5077 | CB | ALA | C | 119 | 41.353 | 33.907 | −14.177 | 1.00 | 22.79 | C |
| ATOM | 5078 | N | SER | C | 120 | 42.567 | 36.897 | −14.977 | 1.00 | 28.58 | N |
| ATOM | 5079 | CA | SER | C | 120 | 42.356 | 38.335 | −15.106 | 1.00 | 30.17 | C |
| ATOM | 5080 | C | SER | C | 120 | 42.446 | 38.771 | −16.558 | 1.00 | 30.24 | C |
| ATOM | 5081 | O | SER | C | 120 | 41.458 | 39.250 | −17.119 | 1.00 | 31.14 | O |
| ATOM | 5082 | CB | SER | C | 120 | 43.374 | 39.127 | −14.283 | 1.00 | 28.09 | C |
| ATOM | 5083 | OG | SER | C | 120 | 43.176 | 38.937 | −12.893 | 1.00 | 39.21 | O |
| ATOM | 5084 | N | ILE | C | 121 | 43.629 | 38.610 | −17.155 | 1.00 | 23.05 | N |
| ATOM | 5085 | CA | ILE | C | 121 | 43.855 | 39.011 | −18.539 | 1.00 | 23.20 | C |
| ATOM | 5086 | C | ILE | C | 121 | 42.824 | 38.335 | −19.443 | 1.00 | 24.90 | C |
| ATOM | 5087 | O | ILE | C | 121 | 42.269 | 38.962 | −20.342 | 1.00 | 30.34 | O |
| ATOM | 5088 | CB | ILE | C | 121 | 45.315 | 38.736 | −19.009 | 1.00 | 22.40 | C |
| ATOM | 5089 | CG1 | ILE | C | 121 | 45.663 | 39.537 | −20.267 | 1.00 | 24.10 | C |
| ATOM | 5090 | CG2 | ILE | C | 121 | 45.541 | 37.262 | −19.283 | 1.00 | 22.46 | C |
| ATOM | 5091 | CD1 | ILE | C | 121 | 45.255 | 40.976 | −20.211 | 1.00 | 31.26 | C |
| ATOM | 5092 | N | GLU | C | 122 | 42.534 | 37.067 | −19.185 | 1.00 | 27.17 | N |
| ATOM | 5093 | CA | GLU | C | 122 | 41.527 | 36.369 | −19.980 | 1.00 | 28.10 | C |
| ATOM | 5094 | C | GLU | C | 122 | 40.176 | 37.048 | −19.865 | 1.00 | 29.87 | C |
| ATOM | 5095 | O | GLU | C | 122 | 39.465 | 37.176 | −20.856 | 1.00 | 32.92 | O |
| ATOM | 5096 | CB | GLU | C | 122 | 41.409 | 34.897 | −19.581 | 1.00 | 25.66 | C |
| ATOM | 5097 | CG | GLU | C | 122 | 42.333 | 33.969 | −20.342 | 1.00 | 27.36 | C |
| ATOM | 5098 | CD | GLU | C | 122 | 42.033 | 32.497 | −20.078 | 1.00 | 43.44 | C |
| ATOM | 5099 | OE1 | GLU | C | 122 | 40.943 | 32.195 | −19.535 | 1.00 | 42.49 | O |
| ATOM | 5100 | OE2 | GLU | C | 122 | 42.884 | 31.640 | −20.417 | 1.00 | 43.62 | O |
| ATOM | 5101 | N | THR | C | 123 | 39.820 | 37.477 | −18.658 | 1.00 | 31.11 | N |
| ATOM | 5102 | CA | THR | C | 123 | 38.556 | 38.174 | −18.445 | 1.00 | 27.09 | C |
| ATOM | 5103 | C | THR | C | 123 | 38.561 | 39.557 | −19.091 | 1.00 | 30.79 | C |
| ATOM | 5104 | O | THR | C | 123 | 37.600 | 39.945 | −19.731 | 1.00 | 31.71 | O |
| ATOM | 5105 | CB | THR | C | 123 | 38.217 | 38.294 | −16.952 | 1.00 | 32.70 | C |
| ATOM | 5106 | OG1 | THR | C | 123 | 37.853 | 37.004 | −16.442 | 1.00 | 34.25 | O |
| ATOM | 5107 | CG2 | THR | C | 123 | 37.063 | 39.260 | −16.734 | 1.00 | 30.31 | C |
| ATOM | 5108 | N | LEU | C | 124 | 39.649 | 40.297 | −18.935 | 1.00 | 30.24 | N |
| ATOM | 5109 | CA | LEU | C | 124 | 39.759 | 41.592 | −19.581 | 1.00 | 29.95 | C |
| ATOM | 5110 | C | LEU | C | 124 | 39.564 | 41.447 | −21.082 | 1.00 | 35.53 | C |
| ATOM | 5111 | O | LEU | C | 124 | 39.079 | 42.352 | −21.752 | 1.00 | 44.29 | O |
| ATOM | 5112 | CB | LEU | C | 124 | 41.113 | 42.228 | −19.275 | 1.00 | 33.34 | C |
| ATOM | 5113 | CG | LEU | C | 124 | 41.166 | 42.798 | −17.858 | 1.00 | 28.66 | C |
| ATOM | 5114 | CD1 | LEU | C | 124 | 42.592 | 43.183 | −17.449 | 1.00 | 31.00 | C |
| ATOM | 5115 | CD2 | LEU | C | 124 | 40.240 | 43.977 | −17.774 | 1.00 | 31.04 | C |
| ATOM | 5116 | N | CYS | C | 125 | 39.940 | 40.294 | −21.609 | 1.00 | 35.81 | N |
| ATOM | 5117 | CA | CYS | C | 125 | 39.780 | 40.030 | −23.024 | 1.00 | 36.50 | C |
| ATOM | 5118 | C | CYS | C | 125 | 38.296 | 39.991 | −23.338 | 1.00 | 36.65 | C |
| ATOM | 5119 | O | CYS | C | 125 | 37.795 | 40.736 | −24.186 | 1.00 | 39.03 | O |
| ATOM | 5120 | CB | CYS | C | 125 | 40.415 | 38.688 | −23.370 | 1.00 | 35.76 | C |
| ATOM | 5121 | SG | CYS | C | 125 | 41.535 | 38.785 | −24.745 | 1.00 | 47.66 | S |
| ATOM | 5122 | N | VAL | C | 126 | 37.592 | 39.116 | −22.636 | 1.00 | 33.56 | N |
| ATOM | 5123 | CA | VAL | C | 126 | 36.158 | 38.986 | −22.808 | 1.00 | 29.67 | C |
| ATOM | 5124 | C | VAL | C | 126 | 35.449 | 40.340 | −22.703 | 1.00 | 33.19 | C |
| ATOM | 5125 | O | VAL | C | 126 | 34.646 | 40.676 | −23.564 | 1.00 | 40.38 | O |
| ATOM | 5126 | CB | VAL | C | 126 | 35.578 | 37.948 | −21.832 | 1.00 | 31.47 | C |
| ATOM | 5127 | CG1 | VAL | C | 126 | 34.050 | 38.045 | −21.757 | 1.00 | 34.32 | C |
| ATOM | 5128 | CG2 | VAL | C | 126 | 36.009 | 36.557 | −22.254 | 1.00 | 24.96 | C |
| ATOM | 5129 | N | ILE | C | 127 | 35.762 | 41.123 | −21.673 | 1.00 | 31.75 | N |
| ATOM | 5130 | CA | ILE | C | 127 | 35.217 | 42.481 | −21.532 | 1.00 | 34.41 | C |
| ATOM | 5131 | C | ILE | C | 127 | 35.368 | 43.301 | −22.817 | 1.00 | 39.24 | C |
| ATOM | 5132 | O | ILE | C | 127 | 34.425 | 43.952 | −23.257 | 1.00 | 39.02 | O |
| ATOM | 5133 | CB | ILE | C | 127 | 35.892 | 43.267 | −20.376 | 1.00 | 45.70 | C |
| ATOM | 5134 | CG1 | ILE | C | 127 | 35.440 | 42.751 | −19.013 | 1.00 | 32.84 | C |
| ATOM | 5135 | CG2 | ILE | C | 127 | 35.603 | 44.767 | −20.472 | 1.00 | 35.85 | C |
| ATOM | 5136 | CD1 | ILE | C | 127 | 36.264 | 43.337 | −17.884 | 1.00 | 37.36 | C |
| ATOM | 5137 | N | ALA | C | 128 | 36.552 | 43.270 | −23.419 | 1.00 | 33.41 | N |
| ATOM | 5138 | CA | ALA | C | 128 | 36.794 | 44.048 | −24.626 | 1.00 | 36.54 | C |
| ATOM | 5139 | C | ALA | C | 128 | 35.979 | 43.534 | −25.816 | 1.00 | 44.44 | C |
| ATOM | 5140 | O | ALA | C | 128 | 35.289 | 44.310 | −26.490 | 1.00 | 49.06 | O |
| ATOM | 5141 | CB | ALA | C | 128 | 38.283 | 44.081 | −24.961 | 1.00 | 38.07 | C |
| ATOM | 5142 | N | ILE | C | 129 | 36.070 | 42.229 | −26.069 | 1.00 | 33.39 | N |
| ATOM | 5143 | CA | ILE | C | 129 | 35.333 | 41.598 | −27.159 | 1.00 | 36.73 | C |
| ATOM | 5144 | C | ILE | C | 129 | 33.817 | 41.783 | −27.029 | 1.00 | 39.32 | C |
| ATOM | 5145 | O | ILE | C | 129 | 33.112 | 41.914 | −28.027 | 1.00 | 35.89 | O |
| ATOM | 5146 | CB | ILE | C | 129 | 35.628 | 40.094 | −27.219 | 1.00 | 41.68 | C |
| ATOM | 5147 | CG1 | ILE | C | 129 | 37.066 | 39.855 | −27.673 | 1.00 | 39.85 | C |
| ATOM | 5148 | CG2 | ILE | C | 129 | 34.630 | 39.387 | −28.138 | 1.00 | 33.75 | C |
| ATOM | 5149 | CD1 | ILE | C | 129 | 37.490 | 38.410 | −27.582 | 1.00 | 28.06 | C |
| ATOM | 5150 | N | ASP | C | 130 | 33.330 | 41.773 | −25.790 | 1.00 | 40.86 | N |
| ATOM | 5151 | CA | ASP | C | 130 | 31.919 | 41.969 | −25.485 | 1.00 | 37.27 | C |
| ATOM | 5152 | C | ASP | C | 130 | 31.480 | 43.369 | −25.890 | 1.00 | 40.65 | C |
| ATOM | 5153 | O | ASP | C | 130 | 30.484 | 43.539 | −26.588 | 1.00 | 38.96 | O |
| ATOM | 5154 | CB | ASP | C | 130 | 31.688 | 41.747 | −23.985 | 1.00 | 42.29 | C |
| ATOM | 5155 | CG | ASP | C | 130 | 30.522 | 42.570 | −23.425 | 1.00 | 56.32 | C |
| ATOM | 5156 | OD1 | ASP | C | 130 | 29.377 | 42.359 | −23.873 | 1.00 | 66.96 | O |
| ATOM | 5157 | OD2 | ASP | C | 130 | 30.749 | 43.408 | −22.516 | 1.00 | 53.17 | O |
| ATOM | 5158 | N | ARG | C | 131 | 32.234 | 44.364 | −25.438 | 1.00 | 42.60 | N |
| ATOM | 5159 | CA | ARG | C | 131 | 31.996 | 45.752 | −25.801 | 1.00 | 48.66 | C |
| ATOM | 5160 | C | ARG | C | 131 | 32.043 | 45.940 | −27.310 | 1.00 | 42.20 | C |
| ATOM | 5161 | O | ARG | C | 131 | 31.173 | 46.591 | −27.891 | 1.00 | 40.78 | O |
| ATOM | 5162 | CB | ARG | C | 131 | 33.028 | 46.665 | −25.133 | 1.00 | 47.37 | C |
| ATOM | 5163 | CG | ARG | C | 131 | 32.807 | 46.863 | −23.639 | 1.00 | 40.25 | C |
| ATOM | 5164 | CD | ARG | C | 131 | 31.416 | 47.389 | −23.349 | 1.00 | 37.07 | C |
| ATOM | 5165 | NE | ARG | C | 131 | 30.418 | 46.328 | −23.276 | 1.00 | 50.00 | N |
| ATOM | 5166 | CZ | ARG | C | 131 | 29.106 | 46.544 | −23.196 | 1.00 | 59.17 | C |
| ATOM | 5167 | NH1 | ARG | C | 131 | 28.635 | 47.787 | −23.195 | 1.00 | 56.73 | N |
| ATOM | 5168 | NH2 | ARG | C | 131 | 28.261 | 45.519 | −23.127 | 1.00 | 44.45 | N |
| ATOM | 5169 | N | TYR | C | 132 | 33.058 | 45.367 | −27.942 | 1.00 | 36.06 | N |
| ATOM | 5170 | CA | TYR | C | 132 | 33.188 | 45.492 | −29.389 | 1.00 | 50.69 | C |
| ATOM | 5171 | C | TYR | C | 132 | 32.004 | 44.899 | −30.159 | 1.00 | 51.94 | C |
| ATOM | 5172 | O | TYR | C | 132 | 31.674 | 45.366 | −31.250 | 1.00 | 53.20 | O |
| ATOM | 5173 | CB | TYR | C | 132 | 34.492 | 44.878 | −29.889 | 1.00 | 44.59 | C |
| ATOM | 5174 | CG | TYR | C | 132 | 34.580 | 44.883 | −31.391 | 1.00 | 43.69 | C |
| ATOM | 5175 | CD1 | TYR | C | 132 | 35.074 | 45.986 | −32.074 | 1.00 | 52.80 | C |
| ATOM | 5176 | CD2 | TYR | C | 132 | 34.148 | 43.790 | −32.129 | 1.00 | 50.07 | C |
| ATOM | 5177 | CE1 | TYR | C | 132 | 35.147 | 45.996 | −33.446 | 1.00 | 56.18 | C |
| ATOM | 5178 | CE2 | TYR | C | 132 | 34.218 | 43.788 | −33.496 | 1.00 | 56.67 | C |
| ATOM | 5179 | CZ | TYR | C | 132 | 34.713 | 44.893 | −34.154 | 1.00 | 61.49 | C |
| ATOM | 5180 | OH | TYR | C | 132 | 34.772 | 44.884 | −35.527 | 1.00 | 68.89 | O |
| ATOM | 5181 | N | LEU | C | 133 | 31.379 | 43.866 | −29.606 | 1.00 | 39.89 | N |
| ATOM | 5182 | CA | LEU | C | 133 | 30.176 | 43.309 | −30.216 | 1.00 | 43.18 | C |
| ATOM | 5183 | C | LEU | C | 133 | 28.948 | 44.184 | −29.936 | 1.00 | 46.36 | C |
| ATOM | 5184 | O | LEU | C | 133 | 28.126 | 44.412 | −30.821 | 1.00 | 46.64 | O |
| ATOM | 5185 | CB | LEU | C | 133 | 29.920 | 41.880 | −29.737 | 1.00 | 33.56 | C |
| ATOM | 5186 | CG | LEU | C | 133 | 30.940 | 40.798 | −30.106 | 1.00 | 41.93 | C |
| ATOM | 5187 | CD1 | LEU | C | 133 | 30.559 | 39.491 | −29.400 | 1.00 | 33.81 | C |
| ATOM | 5188 | CD2 | LEU | C | 133 | 31.078 | 40.593 | −31.621 | 1.00 | 28.13 | C |
| ATOM | 5189 | N | ALA | C | 134 | 28.823 | 44.669 | −28.706 | 1.00 | 46.69 | N |
| ATOM | 5190 | CA | ALA | C | 134 | 27.684 | 45.494 | −28.342 | 1.00 | 42.61 | C |
| ATOM | 5191 | C | ALA | C | 134 | 27.689 | 46.743 | −29.199 | 1.00 | 43.95 | C |
| ATOM | 5192 | O | ALA | C | 134 | 26.649 | 47.192 | −29.671 | 1.00 | 54.21 | O |
| ATOM | 5193 | CB | ALA | C | 134 | 27.730 | 45.857 | −26.873 | 1.00 | 29.76 | C |
| ATOM | 5194 | N | ILE | C | 135 | 28.874 | 47.282 | −29.428 | 1.00 | 50.79 | N |
| ATOM | 5195 | CA | ILE | C | 135 | 29.001 | 48.541 | −30.143 | 1.00 | 55.92 | C |
| ATOM | 5196 | C | ILE | C | 135 | 28.780 | 48.403 | −31.659 | 1.00 | 55.52 | C |
| ATOM | 5197 | O | ILE | C | 135 | 28.214 | 49.302 | −32.277 | 1.00 | 63.61 | O |
| ATOM | 5198 | CB | ILE | C | 135 | 30.359 | 49.202 | −29.861 | 1.00 | 58.21 | C |
| ATOM | 5199 | CG1 | ILE | C | 135 | 30.169 | 50.635 | −29.380 | 1.00 | 51.90 | C |
| ATOM | 5200 | CG2 | ILE | C | 135 | 31.260 | 49.147 | −31.085 | 1.00 | 63.93 | C |
| ATOM | 5201 | CD1 | ILE | C | 135 | 31.483 | 51.349 | −29.136 | 1.00 | 66.88 | C |
| ATOM | 5202 | N | THR | C | 136 | 29.206 | 47.287 | −32.254 | 1.00 | 52.09 | N |
| ATOM | 5203 | CA | THR | C | 136 | 29.015 | 47.077 | −33.697 | 1.00 | 46.61 | C |
| ATOM | 5204 | C | THR | C | 136 | 27.734 | 46.322 | −34.062 | 1.00 | 52.82 | C |
| ATOM | 5205 | O | THR | C | 136 | 27.143 | 46.598 | −35.102 | 1.00 | 61.96 | O |
| ATOM | 5206 | CB | THR | C | 136 | 30.212 | 46.351 | −34.381 | 1.00 | 51.99 | C |
| ATOM | 5207 | OG1 | THR | C | 136 | 30.191 | 44.952 | −34.056 | 1.00 | 45.16 | O |
| ATOM | 5208 | CG2 | THR | C | 136 | 31.558 | 46.982 | −33.998 | 1.00 | 45.18 | C |
| ATOM | 5209 | N | SER | C | 137 | 27.302 | 45.377 | −33.227 | 1.00 | 52.87 | N |
| ATOM | 5210 | CA | SER | C | 137 | 26.137 | 44.541 | −33.556 | 1.00 | 49.57 | C |
| ATOM | 5211 | C | SER | C | 137 | 24.981 | 44.611 | −32.559 | 1.00 | 51.96 | C |
| ATOM | 5212 | O | SER | C | 137 | 24.434 | 43.578 | −32.183 | 1.00 | 51.48 | O |
| ATOM | 5213 | CB | SER | C | 137 | 26.556 | 43.077 | −33.700 | 1.00 | 41.53 | C |
| ATOM | 5214 | OG | SER | C | 137 | 27.431 | 42.899 | −34.796 | 1.00 | 62.51 | O |
| ATOM | 5215 | N | PRO | C | 138 | 24.585 | 45.824 | −32.150 | 1.00 | 55.98 | N |
| ATOM | 5216 | CA | PRO | C | 138 | 23.587 | 46.003 | −31.086 | 1.00 | 53.71 | C |
| ATOM | 5217 | C | PRO | C | 138 | 22.386 | 45.055 | −31.156 | 1.00 | 60.77 | C |
| ATOM | 5218 | O | PRO | C | 138 | 21.975 | 44.556 | −30.112 | 1.00 | 65.02 | O |
| ATOM | 5219 | CB | PRO | C | 138 | 23.133 | 47.459 | −31.275 | 1.00 | 39.21 | C |
| ATOM | 5220 | CG | PRO | C | 138 | 23.695 | 47.868 | −32.625 | 1.00 | 61.71 | C |
| ATOM | 5221 | CD | PRO | C | 138 | 24.974 | 47.121 | −32.720 | 1.00 | 45.27 | C |
| ATOM | 5222 | N | PHE | C | 139 | 21.831 | 44.813 | −32.341 | 1.00 | 62.93 | N |
| ATOM | 5223 | CA | PHE | C | 139 | 20.649 | 43.954 | −32.448 | 1.00 | 64.31 | C |
| ATOM | 5224 | C | PHE | C | 139 | 20.987 | 42.477 | −32.279 | 1.00 | 64.55 | C |
| ATOM | 5225 | O | PHE | C | 139 | 20.323 | 41.765 | −31.519 | 1.00 | 69.43 | O |
| ATOM | 5226 | CB | PHE | C | 139 | 19.897 | 44.177 | −33.768 | 1.00 | 83.98 | C |
| ATOM | 5227 | CG | PHE | C | 139 | 18.618 | 43.382 | −33.879 | 1.00 | 95.82 | C |
| ATOM | 5228 | CD1 | PHE | C | 139 | 17.420 | 43.891 | −33.391 | 1.00 | 94.78 | C |
| ATOM | 5229 | CD2 | PHE | C | 139 | 18.615 | 42.120 | −34.462 | 1.00 | 89.49 | C |
| ATOM | 5230 | CE1 | PHE | C | 139 | 16.245 | 43.159 | −33.484 | 1.00 | 85.31 | C |
| ATOM | 5231 | CE2 | PHE | C | 139 | 17.442 | 41.382 | −34.554 | 1.00 | 85.42 | C |
| ATOM | 5232 | CZ | PHE | C | 139 | 16.258 | 41.904 | −34.064 | 1.00 | 82.57 | C |
| ATOM | 5233 | N | ARG | C | 140 | 22.012 | 42.011 | −32.989 | 1.00 | 60.87 | N |
| ATOM | 5234 | CA | ARG | C | 140 | 22.449 | 40.625 | −32.842 | 1.00 | 60.29 | C |
| ATOM | 5235 | C | ARG | C | 140 | 23.063 | 40.364 | −31.468 | 1.00 | 61.68 | C |
| ATOM | 5236 | O | ARG | C | 140 | 23.251 | 39.215 | −31.074 | 1.00 | 69.06 | O |
| ATOM | 5237 | CB | ARG | C | 140 | 23.425 | 40.231 | −33.945 | 1.00 | 55.75 | C |
| ATOM | 5238 | CG | ARG | C | 140 | 22.756 | 39.948 | −35.273 | 1.00 | 62.53 | C |
| ATOM | 5239 | CD | ARG | C | 140 | 23.746 | 39.389 | −36.272 | 1.00 | 70.85 | C |
| ATOM | 5240 | NE | ARG | C | 140 | 23.082 | 38.920 | −37.482 | 1.00 | 85.03 | N |
| ATOM | 5241 | CZ | ARG | C | 140 | 23.689 | 38.228 | −38.440 | 1.00 | 90.43 | C |
| ATOM | 5242 | NH1 | ARG | C | 140 | 24.974 | 37.929 | −38.318 | 1.00 | 92.11 | N |
| ATOM | 5243 | NH2 | ARG | C | 140 | 23.016 | 37.835 | −39.517 | 1.00 | 84.82 | N |
| ATOM | 5244 | N | TYR | C | 141 | 23.354 | 41.433 | −30.734 | 1.00 | 58.53 | N |
| ATOM | 5245 | CA | TYR | C | 141 | 23.923 | 41.308 | −29.397 | 1.00 | 52.86 | C |
| ATOM | 5246 | C | TYR | C | 141 | 22.867 | 41.074 | −28.308 | 1.00 | 64.20 | C |
| ATOM | 5247 | O | TYR | C | 141 | 22.998 | 40.142 | −27.515 | 1.00 | 66.32 | O |
| ATOM | 5248 | CB | TYR | C | 141 | 24.778 | 42.533 | −29.067 | 1.00 | 54.37 | C |
| ATOM | 5249 | CG | TYR | C | 141 | 25.363 | 42.517 | −27.680 | 1.00 | 53.21 | C |
| ATOM | 5250 | CD1 | TYR | C | 141 | 26.575 | 41.886 | −27.418 | 1.00 | 62.39 | C |
| ATOM | 5251 | CD2 | TYR | C | 141 | 24.701 | 43.132 | −26.626 | 1.00 | 65.34 | C |
| ATOM | 5252 | CE1 | TYR | C | 141 | 27.112 | 41.869 | −26.137 | 1.00 | 58.10 | C |
| ATOM | 5253 | CE2 | TYR | C | 141 | 25.226 | 43.123 | −25.347 | 1.00 | 64.84 | C |
| ATOM | 5254 | CZ | TYR | C | 141 | 26.428 | 42.493 | −25.107 | 1.00 | 60.66 | C |
| ATOM | 5255 | OH | TYR | C | 141 | 26.932 | 42.495 | −23.825 | 1.00 | 63.54 | O |
| ATOM | 5256 | N | GLN | C | 142 | 21.831 | 41.913 | −28.274 | 1.00 | 61.43 | N |
| ATOM | 5257 | CA | GLN | C | 142 | 20.763 | 41.812 | −27.273 | 1.00 | 67.69 | C |
| ATOM | 5258 | C | GLN | C | 142 | 20.123 | 40.428 | −27.183 | 1.00 | 73.75 | C |
| ATOM | 5259 | O | GLN | C | 142 | 19.857 | 39.920 | −26.086 | 1.00 | 72.00 | O |
| ATOM | 5260 | CB | GLN | C | 142 | 19.655 | 42.819 | −27.572 | 1.00 | 89.63 | C |
| ATOM | 5261 | CG | GLN | C | 142 | 19.919 | 44.241 | −27.131 | 1.00 | 100.22 | C |
| ATOM | 5262 | CD | GLN | C | 142 | 18.805 | 45.180 | −27.567 | 1.00 | 121.31 | C |
| ATOM | 5263 | OE1 | GLN | C | 142 | 17.651 | 44.765 | −27.733 | 1.00 | 98.60 | O |
| ATOM | 5264 | NE2 | GLN | C | 142 | 19.147 | 46.450 | −27.763 | 1.00 | 124.72 | N |
| ATOM | 5265 | N | SER | C | 143 | 19.850 | 39.832 | −28.341 | 1.00 | 75.52 | N |
| ATOM | 5266 | CA | SER | C | 143 | 19.154 | 38.548 | −28.395 | 1.00 | 74.63 | C |
| ATOM | 5267 | C | SER | C | 143 | 20.040 | 37.391 | −27.948 | 1.00 | 70.33 | C |
| ATOM | 5268 | O | SER | C | 143 | 19.588 | 36.505 | −27.219 | 1.00 | 76.73 | O |
| ATOM | 5269 | CB | SER | C | 143 | 18.562 | 38.292 | −29.792 | 1.00 | 78.24 | C |
| ATOM | 5270 | OG | SER | C | 143 | 19.232 | 39.038 | −30.798 | 1.00 | 69.78 | O |
| ATOM | 5271 | N | LEU | C | 144 | 21.302 | 37.413 | −28.365 | 1.00 | 58.65 | N |
| ATOM | 5272 | CA | LEU | C | 144 | 22.230 | 36.341 | −28.017 | 1.00 | 62.72 | C |
| ATOM | 5273 | C | LEU | C | 144 | 22.778 | 36.420 | −26.587 | 1.00 | 62.92 | C |
| ATOM | 5274 | O | LEU | C | 144 | 22.989 | 35.398 | −25.939 | 1.00 | 57.27 | O |
| ATOM | 5275 | CB | LEU | C | 144 | 23.371 | 36.272 | −29.034 | 1.00 | 56.96 | C |
| ATOM | 5276 | CG | LEU | C | 144 | 22.823 | 35.930 | −30.420 | 1.00 | 68.90 | C |
| ATOM | 5277 | CD1 | LEU | C | 144 | 23.919 | 35.470 | −31.367 | 1.00 | 48.42 | C |
| ATOM | 5278 | CD2 | LEU | C | 144 | 21.741 | 34.864 | −30.289 | 1.00 | 56.67 | C |
| ATOM | 5279 | N | MET | C | 145 | 22.994 | 37.629 | −26.086 | 1.00 | 55.61 | N |
| ATOM | 5280 | CA | MET | C | 145 | 23.630 | 37.785 | −24.781 | 1.00 | 58.58 | C |
| ATOM | 5281 | C | MET | C | 145 | 22.669 | 38.017 | −23.610 | 1.00 | 53.92 | C |
| ATOM | 5282 | O | MET | C | 145 | 22.148 | 39.112 | −23.432 | 1.00 | 54.95 | O |
| ATOM | 5283 | CB | MET | C | 145 | 24.678 | 38.899 | −24.838 | 1.00 | 56.51 | C |
| ATOM | 5284 | CG | MET | C | 145 | 26.067 | 38.428 | −25.269 | 1.00 | 69.80 | C |
| ATOM | 5285 | SD | MET | C | 145 | 27.180 | 38.084 | −23.874 | 1.00 | 81.42 | S |
| ATOM | 5286 | CE | MET | C | 145 | 26.487 | 36.565 | −23.218 | 1.00 | 54.05 | C |
| ATOM | 5287 | N | THR | C | 146 | 22.441 | 36.972 | −22.821 | 1.00 | 46.04 | N |
| ATOM | 5288 | CA | THR | C | 146 | 21.707 | 37.094 | −21.568 | 1.00 | 45.12 | C |
| ATOM | 5289 | C | THR | C | 146 | 22.691 | 37.029 | −20.394 | 1.00 | 51.33 | C |
| ATOM | 5290 | O | THR | C | 146 | 23.903 | 37.029 | −20.604 | 1.00 | 55.24 | O |
| ATOM | 5291 | CB | THR | C | 146 | 20.622 | 35.998 | −21.428 | 1.00 | 56.69 | C |
| ATOM | 5292 | OG1 | THR | C | 146 | 21.231 | 34.698 | −21.463 | 1.00 | 51.03 | O |
| ATOM | 5293 | CG2 | THR | C | 146 | 19.604 | 36.110 | −22.555 | 1.00 | 54.71 | C |
| ATOM | 5294 | N | ARG | C | 147 | 22.180 | 36.978 | −19.164 | 1.00 | 51.50 | N |
| ATOM | 5295 | CA | ARG | C | 147 | 23.051 | 36.906 | −17.989 | 1.00 | 41.33 | C |
| ATOM | 5296 | C | ARG | C | 147 | 23.474 | 35.474 | −17.678 | 1.00 | 44.42 | C |
| ATOM | 5297 | O | ARG | C | 147 | 24.560 | 35.235 | −17.152 | 1.00 | 48.82 | O |
| ATOM | 5298 | CB | ARG | C | 147 | 22.378 | 37.515 | −16.756 | 1.00 | 56.58 | C |
| ATOM | 5299 | CG | ARG | C | 147 | 22.235 | 39.018 | −16.790 | 1.00 | 68.10 | C |
| ATOM | 5300 | CD | ARG | C | 147 | 21.709 | 39.524 | −15.464 | 1.00 | 73.30 | C |
| ATOM | 5301 | NE | ARG | C | 147 | 21.190 | 40.878 | −15.583 | 1.00 | 85.24 | N |
| ATOM | 5302 | CZ | ARG | C | 147 | 20.273 | 41.390 | −14.773 | 1.00 | 88.04 | C |
| ATOM | 5303 | NH1 | ARG | C | 147 | 19.780 | 40.651 | −13.790 | 1.00 | 92.61 | N |
| ATOM | 5304 | NH2 | ARG | C | 147 | 19.846 | 42.634 | −14.949 | 1.00 | 84.03 | N |
| ATOM | 5305 | N | ALA | C | 148 | 22.613 | 34.518 | −17.996 | 1.00 | 48.93 | N |
| ATOM | 5306 | CA | ALA | C | 148 | 22.941 | 33.124 | −17.745 | 1.00 | 51.60 | C |
| ATOM | 5307 | C | ALA | C | 148 | 24.169 | 32.739 | −18.563 | 1.00 | 41.90 | C |
| ATOM | 5308 | O | ALA | C | 148 | 24.961 | 31.883 | −18.157 | 1.00 | 41.02 | O |
| ATOM | 5309 | CB | ALA | C | 148 | 21.761 | 32.227 | −18.076 | 1.00 | 35.92 | C |
| ATOM | 5310 | N | ARG | C | 149 | 24.326 | 33.399 | −19.705 | 1.00 | 32.02 | N |
| ATOM | 5311 | CA | ARG | C | 149 | 25.438 | 33.115 | −20.602 | 1.00 | 49.50 | C |
| ATOM | 5312 | C | ARG | C | 149 | 26.758 | 33.772 | −20.204 | 1.00 | 46.90 | C |
| ATOM | 5313 | O | ARG | C | 149 | 27.829 | 33.172 | −20.357 | 1.00 | 35.31 | O |
| ATOM | 5314 | CB | ARG | C | 149 | 25.066 | 33.455 | −22.042 | 1.00 | 47.87 | C |
| ATOM | 5315 | CG | ARG | C | 149 | 24.204 | 32.383 | −22.654 | 1.00 | 49.50 | C |
| ATOM | 5316 | CD | ARG | C | 149 | 23.805 | 32.689 | −24.070 | 1.00 | 52.59 | C |
| ATOM | 5317 | NE | ARG | C | 149 | 22.734 | 31.784 | −24.466 | 1.00 | 59.22 | N |
| ATOM | 5318 | CZ | ARG | C | 149 | 21.836 | 32.053 | −25.403 | 1.00 | 59.63 | C |
| ATOM | 5319 | NH1 | ARG | C | 149 | 21.880 | 33.210 | −26.053 | 1.00 | 57.37 | N |
| ATOM | 5320 | NH2 | ARG | C | 149 | 20.893 | 31.166 | −25.684 | 1.00 | 70.46 | N |
| ATOM | 5321 | N | ALA | C | 150 | 26.682 | 34.996 | −19.695 | 1.00 | 38.49 | N |
| ATOM | 5322 | CA | ALA | C | 150 | 27.861 | 35.639 | −19.146 | 1.00 | 38.00 | C |
| ATOM | 5323 | C | ALA | C | 150 | 28.436 | 34.789 | −18.006 | 1.00 | 37.45 | C |
| ATOM | 5324 | O | ALA | C | 150 | 29.655 | 34.606 | −17.906 | 1.00 | 34.48 | O |
| ATOM | 5325 | CB | ALA | C | 150 | 27.531 | 37.043 | −18.672 | 1.00 | 42.06 | C |
| ATOM | 5326 | N | LYS | C | 151 | 27.563 | 34.259 | −17.152 | 1.00 | 32.72 | N |
| ATOM | 5327 | CA | LYS | C | 151 | 28.011 | 33.334 | −16.117 | 1.00 | 34.60 | C |
| ATOM | 5328 | C | LYS | C | 151 | 28.687 | 32.121 | −16.759 | 1.00 | 40.36 | C |
| ATOM | 5329 | O | LYS | C | 151 | 29.792 | 31.729 | −16.364 | 1.00 | 37.01 | O |
| ATOM | 5330 | CB | LYS | C | 151 | 26.859 | 32.882 | −15.222 | 1.00 | 24.47 | C |
| ATOM | 5331 | CG | LYS | C | 151 | 26.330 | 33.936 | −14.273 | 1.00 | 41.34 | C |
| ATOM | 5332 | CD | LYS | C | 151 | 25.225 | 33.335 | −13.401 | 1.00 | 60.74 | C |
| ATOM | 5333 | CE | LYS | C | 151 | 24.119 | 34.334 | −13.075 | 1.00 | 64.62 | C |
| ATOM | 5334 | NZ | LYS | C | 151 | 22.811 | 33.631 | −12.871 | 1.00 | 66.54 | N |
| ATOM | 5335 | N | VAL | C | 152 | 28.026 | 31.534 | −17.755 | 1.00 | 38.63 | N |
| ATOM | 5336 | CA | VAL | C | 152 | 28.615 | 30.414 | −18.482 | 1.00 | 36.95 | C |
| ATOM | 5337 | C | VAL | C | 152 | 29.957 | 30.818 | −19.105 | 1.00 | 32.22 | C |
| ATOM | 5338 | O | VAL | C | 152 | 30.914 | 30.056 | −19.073 | 1.00 | 30.31 | O |
| ATOM | 5339 | CB | VAL | C | 152 | 27.662 | 29.838 | −19.545 | 1.00 | 35.07 | C |
| ATOM | 5340 | CG1 | VAL | C | 152 | 28.418 | 28.960 | −20.529 | 1.00 | 38.03 | C |
| ATOM | 5341 | CG2 | VAL | C | 152 | 26.577 | 29.039 | −18.876 | 1.00 | 34.35 | C |
| ATOM | 5342 | N | ILE | C | 153 | 30.035 | 32.025 | −19.646 | 1.00 | 28.98 | N |
| ATOM | 5343 | CA | ILE | C | 153 | 31.304 | 32.517 | −20.172 | 1.00 | 32.17 | C |
| ATOM | 5344 | C | ILE | C | 153 | 32.361 | 32.665 | −19.081 | 1.00 | 28.11 | C |
| ATOM | 5345 | O | ILE | C | 153 | 33.489 | 32.203 | −19.243 | 1.00 | 25.63 | O |
| ATOM | 5346 | CB | ILE | C | 153 | 31.141 | 33.856 | −20.902 | 1.00 | 28.28 | C |
| ATOM | 5347 | CG1 | ILE | C | 153 | 30.243 | 33.675 | −22.122 | 1.00 | 31.14 | C |
| ATOM | 5348 | CG2 | ILE | C | 153 | 32.507 | 34.416 | −21.307 | 1.00 | 25.39 | C |
| ATOM | 5349 | CD1 | ILE | C | 153 | 29.962 | 34.942 | −22.865 | 1.00 | 28.36 | C |
| ATOM | 5350 | N | ILE | C | 154 | 32.002 | 33.316 | −17.979 | 1.00 | 25.66 | N |
| ATOM | 5351 | CA | ILE | C | 154 | 32.936 | 33.477 | −16.864 | 1.00 | 32.11 | C |
| ATOM | 5352 | C | ILE | C | 154 | 33.487 | 32.121 | −16.402 | 1.00 | 32.41 | C |
| ATOM | 5353 | O | ILE | C | 154 | 34.701 | 31.929 | −16.270 | 1.00 | 24.04 | O |
| ATOM | 5354 | CB | ILE | C | 154 | 32.286 | 34.211 | −15.676 | 1.00 | 28.97 | C |
| ATOM | 5355 | CG1 | ILE | C | 154 | 32.239 | 35.720 | −15.952 | 1.00 | 35.49 | C |
| ATOM | 5356 | CG2 | ILE | C | 154 | 33.060 | 33.930 | −14.400 | 1.00 | 21.81 | C |
| ATOM | 5357 | CD1 | ILE | C | 154 | 31.095 | 36.455 | −15.254 | 1.00 | 29.02 | C |
| ATOM | 5358 | N | CYS | C | 155 | 32.585 | 31.174 | −16.176 | 1.00 | 28.52 | N |
| ATOM | 5359 | CA | CYS | C | 155 | 32.987 | 29.827 | −15.809 | 1.00 | 24.48 | C |
| ATOM | 5360 | C | CYS | C | 155 | 33.971 | 29.196 | −16.792 | 1.00 | 32.40 | C |
| ATOM | 5361 | O | CYS | C | 155 | 34.907 | 28.508 | −16.387 | 1.00 | 35.16 | O |
| ATOM | 5362 | CB | CYS | C | 155 | 31.761 | 28.943 | −15.659 | 1.00 | 19.97 | C |
| ATOM | 5363 | SG | CYS | C | 155 | 30.897 | 29.287 | −14.147 | 1.00 | 48.12 | S |
| ATOM | 5364 | N | THR | C | 156 | 33.753 | 29.423 | −18.083 | 1.00 | 28.13 | N |
| ATOM | 5365 | CA | THR | C | 156 | 34.612 | 28.849 | −19.104 | 1.00 | 29.38 | C |
| ATOM | 5366 | C | THR | C | 156 | 36.013 | 29.479 | −19.074 | 1.00 | 23.96 | C |
| ATOM | 5367 | O | THR | C | 156 | 37.010 | 28.779 | −19.189 | 1.00 | 21.72 | O |
| ATOM | 5368 | CB | THR | C | 156 | 33.952 | 28.915 | −20.498 | 1.00 | 22.69 | C |
| ATOM | 5369 | OG1 | THR | C | 156 | 32.681 | 28.269 | −20.426 | 1.00 | 37.62 | O |
| ATOM | 5370 | CG2 | THR | C | 156 | 34.789 | 28.179 | −21.531 | 1.00 | 19.20 | C |
| ATOM | 5371 | N | VAL | C | 157 | 36.084 | 30.789 | −18.881 | 1.00 | 21.52 | N |
| ATOM | 5372 | CA | VAL | C | 157 | 37.373 | 31.434 | −18.652 | 1.00 | 29.93 | C |
| ATOM | 5373 | C | VAL | C | 157 | 38.132 | 30.841 | −17.436 | 1.00 | 28.17 | C |
| ATOM | 5374 | O | VAL | C | 157 | 39.334 | 30.589 | −17.511 | 1.00 | 28.85 | O |
| ATOM | 5375 | CB | VAL | C | 157 | 37.239 | 32.974 | −18.540 | 1.00 | 22.91 | C |
| ATOM | 5376 | CG1 | VAL | C | 157 | 38.554 | 33.617 | −18.066 | 1.00 | 19.45 | C |
| ATOM | 5377 | CG2 | VAL | C | 157 | 36.819 | 33.550 | −19.870 | 1.00 | 20.63 | C |
| ATOM | 5378 | N | TRP | C | 158 | 37.445 | 30.596 | −16.328 | 1.00 | 19.54 | N |
| ATOM | 5379 | CA | TRP | C | 158 | 38.135 | 30.015 | −15.181 | 1.00 | 21.71 | C |
| ATOM | 5380 | C | TRP | C | 158 | 38.560 | 28.564 | −15.437 | 1.00 | 22.61 | C |
| ATOM | 5381 | O | TRP | C | 158 | 39.521 | 28.094 | −14.849 | 1.00 | 19.12 | O |
| ATOM | 5382 | CB | TRP | C | 158 | 37.308 | 30.144 | −13.888 | 1.00 | 25.05 | C |
| ATOM | 5383 | CG | TRP | C | 158 | 37.359 | 31.528 | −13.259 | 1.00 | 23.18 | C |
| ATOM | 5384 | CD1 | TRP | C | 158 | 36.491 | 32.555 | −13.478 | 1.00 | 21.39 | C |
| ATOM | 5385 | CD2 | TRP | C | 158 | 38.337 | 32.027 | −12.333 | 1.00 | 27.65 | C |
| ATOM | 5386 | NE1 | TRP | C | 158 | 36.862 | 33.657 | −12.754 | 1.00 | 23.44 | N |
| ATOM | 5387 | CE2 | TRP | C | 158 | 37.989 | 33.364 | −12.041 | 1.00 | 26.37 | C |
| ATOM | 5388 | CE3 | TRP | C | 158 | 39.473 | 31.478 | −11.728 | 1.00 | 28.95 | C |
| ATOM | 5389 | CZ2 | TRP | C | 158 | 38.729 | 34.158 | −11.168 | 1.00 | 25.78 | C |
| ATOM | 5390 | CZ3 | TRP | C | 158 | 40.210 | 32.270 | −10.861 | 1.00 | 32.81 | C |
| ATOM | 5391 | CH2 | TRP | C | 158 | 39.836 | 33.596 | −10.592 | 1.00 | 34.60 | C |
| ATOM | 5392 | N | ALA | C | 159 | 37.854 | 27.856 | −16.317 | 1.00 | 22.63 | N |
| ATOM | 5393 | CA | ALA | C | 159 | 38.248 | 26.489 | −16.660 | 1.00 | 22.27 | C |
| ATOM | 5394 | C | ALA | C | 159 | 39.468 | 26.507 | −17.575 | 1.00 | 22.85 | C |
| ATOM | 5395 | O | ALA | C | 159 | 40.447 | 25.802 | −17.344 | 1.00 | 20.45 | O |
| ATOM | 5396 | CB | ALA | C | 159 | 37.101 | 25.737 | −17.318 | 1.00 | 19.37 | C |
| ATOM | 5397 | N | ILE | C | 160 | 39.389 | 27.318 | −18.621 | 1.00 | 23.26 | N |
| ATOM | 5398 | CA | ILE | C | 160 | 40.510 | 27.493 | −19.527 | 1.00 | 26.52 | C |
| ATOM | 5399 | C | ILE | C | 160 | 41.734 | 27.924 | −18.731 | 1.00 | 30.29 | C |
| ATOM | 5400 | O | ILE | C | 160 | 42.838 | 27.417 | −18.948 | 1.00 | 28.49 | O |
| ATOM | 5401 | CB | ILE | C | 160 | 40.195 | 28.510 | −20.644 | 1.00 | 20.72 | C |
| ATOM | 5402 | CG1 | ILE | C | 160 | 39.097 | 27.951 | −21.555 | 1.00 | 26.76 | C |
| ATOM | 5403 | CG2 | ILE | C | 160 | 41.442 | 28.821 | −21.453 | 1.00 | 19.34 | C |
| ATOM | 5404 | CD1 | ILE | C | 160 | 38.508 | 28.961 | −22.549 | 1.00 | 23.67 | C |
| ATOM | 5405 | N | SER | C | 161 | 41.530 | 28.845 | −17.793 | 1.00 | 27.40 | N |
| ATOM | 5406 | CA | SER | C | 161 | 42.635 | 29.336 | −16.975 | 1.00 | 29.20 | C |
| ATOM | 5407 | C | SER | C | 161 | 43.188 | 28.231 | −16.098 | 1.00 | 21.40 | C |
| ATOM | 5408 | O | SER | C | 161 | 44.390 | 28.088 | −15.998 | 1.00 | 19.08 | O |
| ATOM | 5409 | CB | SER | C | 161 | 42.229 | 30.548 | −16.131 | 1.00 | 30.96 | C |
| ATOM | 5410 | OG | SER | C | 161 | 42.010 | 31.691 | −16.940 | 1.00 | 27.21 | O |
| ATOM | 5411 | N | ALA | C | 162 | 42.308 | 27.446 | −15.478 | 1.00 | 22.11 | N |
| ATOM | 5412 | CA | ALA | C | 162 | 42.747 | 26.277 | −14.717 | 1.00 | 25.25 | C |
| ATOM | 5413 | C | ALA | C | 162 | 43.496 | 25.281 | −15.611 | 1.00 | 28.31 | C |
| ATOM | 5414 | O | ALA | C | 162 | 44.506 | 24.692 | −15.208 | 1.00 | 22.76 | O |
| ATOM | 5415 | CB | ALA | C | 162 | 41.571 | 25.594 | −14.042 | 1.00 | 11.39 | C |
| ATOM | 5416 | N | LEU | C | 163 | 42.999 | 25.099 | −16.829 | 1.00 | 23.06 | N |
| ATOM | 5417 | CA | LEU | C | 163 | 43.578 | 24.118 | −17.723 | 1.00 | 24.50 | C |
| ATOM | 5418 | C | LEU | C | 163 | 45.024 | 24.478 | −18.074 | 1.00 | 26.00 | C |
| ATOM | 5419 | O | LEU | C | 163 | 45.928 | 23.655 | −17.924 | 1.00 | 21.79 | O |
| ATOM | 5420 | CB | LEU | C | 163 | 42.728 | 23.963 | −18.985 | 1.00 | 23.29 | C |
| ATOM | 5421 | CG | LEU | C | 163 | 43.252 | 22.940 | −19.997 | 1.00 | 27.50 | C |
| ATOM | 5422 | CD1 | LEU | C | 163 | 43.251 | 21.534 | −19.402 | 1.00 | 26.71 | C |
| ATOM | 5423 | CD2 | LEU | C | 163 | 42.450 | 22.963 | −21.271 | 1.00 | 25.71 | C |
| ATOM | 5424 | N | VAL | C | 164 | 45.237 | 25.717 | −18.505 | 1.00 | 28.36 | N |
| ATOM | 5425 | CA | VAL | C | 164 | 46.531 | 26.143 | −19.045 | 1.00 | 27.80 | C |
| ATOM | 5426 | C | VAL | C | 164 | 47.580 | 26.587 | −18.024 | 1.00 | 31.16 | C |
| ATOM | 5427 | O | VAL | C | 164 | 48.742 | 26.782 | −18.384 | 1.00 | 43.73 | O |
| ATOM | 5428 | CB | VAL | C | 164 | 46.370 | 27.274 | −20.084 | 1.00 | 25.39 | C |
| ATOM | 5429 | CG1 | VAL | C | 164 | 45.413 | 26.844 | −21.162 | 1.00 | 24.52 | C |
| ATOM | 5430 | CG2 | VAL | C | 164 | 45.907 | 28.574 | −19.418 | 1.00 | 20.12 | C |
| ATOM | 5431 | N | SER | C | 165 | 47.192 | 26.746 | −16.764 | 1.00 | 29.66 | N |
| ATOM | 5432 | CA | SER | C | 165 | 48.142 | 27.244 | −15.770 | 1.00 | 33.97 | C |
| ATOM | 5433 | C | SER | C | 165 | 48.065 | 26.629 | −14.371 | 1.00 | 30.41 | C |
| ATOM | 5434 | O | SER | C | 165 | 49.031 | 26.688 | −13.621 | 1.00 | 37.83 | O |
| ATOM | 5435 | CB | SER | C | 165 | 48.114 | 28.782 | −15.695 | 1.00 | 42.80 | C |
| ATOM | 5436 | OG | SER | C | 165 | 46.820 | 29.311 | −15.898 | 1.00 | 43.84 | O |
| ATOM | 5437 | N | PHE | C | 166 | 46.936 | 26.043 | −14.007 | 1.00 | 31.12 | N |
| ATOM | 5438 | CA | PHE | C | 166 | 46.912 | 25.268 | −12.773 | 1.00 | 31.81 | C |
| ATOM | 5439 | C | PHE | C | 166 | 47.455 | 23.854 | −13.010 | 1.00 | 28.48 | C |
| ATOM | 5440 | O | PHE | C | 166 | 48.503 | 23.503 | −12.489 | 1.00 | 31.99 | O |
| ATOM | 5441 | CB | PHE | C | 166 | 45.509 | 25.216 | −12.203 | 1.00 | 28.56 | C |
| ATOM | 5442 | CG | PHE | C | 166 | 45.456 | 24.843 | −10.761 | 1.00 | 25.44 | C |
| ATOM | 5443 | CD1 | PHE | C | 166 | 45.254 | 23.531 | −10.384 | 1.00 | 22.52 | C |
| ATOM | 5444 | CD2 | PHE | C | 166 | 45.571 | 25.815 | −9.775 | 1.00 | 32.27 | C |
| ATOM | 5445 | CE1 | PHE | C | 166 | 45.184 | 23.186 | −9.056 | 1.00 | 27.40 | C |
| ATOM | 5446 | CE2 | PHE | C | 166 | 45.500 | 25.477 | −8.432 | 1.00 | 28.83 | C |
| ATOM | 5447 | CZ | PHE | C | 166 | 45.310 | 24.164 | −8.072 | 1.00 | 33.51 | C |
| ATOM | 5448 | N | LEU | C | 167 | 46.750 | 23.053 | −13.801 | 1.00 | 25.12 | N |
| ATOM | 5449 | CA | LEU | C | 167 | 47.198 | 21.689 | −14.116 | 1.00 | 35.45 | C |
| ATOM | 5450 | C | LEU | C | 167 | 48.704 | 21.480 | −14.385 | 1.00 | 31.35 | C |
| ATOM | 5451 | O | LEU | C | 167 | 49.310 | 20.617 | −13.769 | 1.00 | 34.73 | O |
| ATOM | 5452 | CB | LEU | C | 167 | 46.392 | 21.108 | −15.284 | 1.00 | 35.20 | C |
| ATOM | 5453 | CG | LEU | C | 167 | 44.961 | 20.687 | −14.991 | 1.00 | 44.00 | C |
| ATOM | 5454 | CD1 | LEU | C | 167 | 44.370 | 20.045 | −16.238 | 1.00 | 47.63 | C |
| ATOM | 5455 | CD2 | LEU | C | 167 | 44.940 | 19.722 | −13.827 | 1.00 | 38.71 | C |
| ATOM | 5456 | N | PRO | C | 168 | 49.301 | 22.243 | −15.325 | 1.00 | 31.03 | N |
| ATOM | 5457 | CA | PRO | C | 168 | 50.697 | 21.995 | −15.702 | 1.00 | 29.22 | C |
| ATOM | 5458 | C | PRO | C | 168 | 51.675 | 22.133 | −14.543 | 1.00 | 27.03 | C |
| ATOM | 5459 | O | PRO | C | 168 | 52.598 | 21.322 | −14.426 | 1.00 | 29.00 | O |
| ATOM | 5460 | CB | PRO | C | 168 | 50.972 | 23.078 | −16.749 | 1.00 | 27.54 | C |
| ATOM | 5461 | CG | PRO | C | 168 | 49.644 | 23.435 | −17.285 | 1.00 | 22.08 | C |
| ATOM | 5462 | CD | PRO | C | 168 | 48.720 | 23.339 | −16.122 | 1.00 | 28.92 | C |
| ATOM | 5463 | N | ILE | C | 169 | 51.482 | 23.154 | −13.714 | 1.00 | 24.98 | N |
| ATOM | 5464 | CA | ILE | C | 169 | 52.270 | 23.328 | −12.494 | 1.00 | 28.25 | C |
| ATOM | 5465 | C | ILE | C | 169 | 52.093 | 22.173 | −11.510 | 1.00 | 27.60 | C |
| ATOM | 5466 | O | ILE | C | 169 | 53.058 | 21.658 | −10.954 | 1.00 | 33.49 | O |
| ATOM | 5467 | CB | ILE | C | 169 | 51.934 | 24.656 | −11.803 | 1.00 | 23.02 | C |
| ATOM | 5468 | CG1 | ILE | C | 169 | 52.629 | 25.796 | −12.550 | 1.00 | 24.11 | C |
| ATOM | 5469 | CG2 | ILE | C | 169 | 52.371 | 24.626 | −10.366 | 1.00 | 18.81 | C |
| ATOM | 5470 | CD1 | ILE | C | 169 | 52.194 | 27.177 | −12.178 | 1.00 | 16.50 | C |
| ATOM | 5471 | N | MET | C | 170 | 50.850 | 21.762 | −11.310 | 1.00 | 25.18 | N |
| ATOM | 5472 | CA | MET | C | 170 | 50.555 | 20.588 | −10.508 | 1.00 | 26.69 | C |
| ATOM | 5473 | C | MET | C | 170 | 50.928 | 19.253 | −11.180 | 1.00 | 36.71 | C |
| ATOM | 5474 | O | MET | C | 170 | 50.998 | 18.230 | −10.517 | 1.00 | 37.06 | O |
| ATOM | 5475 | CB | MET | C | 170 | 49.082 | 20.600 | −10.115 | 1.00 | 41.25 | C |
| ATOM | 5476 | CG | MET | C | 170 | 48.666 | 21.838 | −9.292 | 1.00 | 48.56 | C |
| ATOM | 5477 | SD | MET | C | 170 | 48.966 | 21.710 | −7.506 | 1.00 | 44.57 | S |
| ATOM | 5478 | CE | MET | C | 170 | 50.739 | 21.941 | −7.434 | 1.00 | 33.99 | C |
| ATOM | 5479 | N | MET | C | 171 | 51.169 | 19.265 | −12.487 | 1.00 | 37.67 | N |
| ATOM | 5480 | CA | MET | C | 171 | 51.657 | 18.084 | −13.191 | 1.00 | 39.60 | C |
| ATOM | 5481 | C | MET | C | 171 | 53.193 | 18.051 | −13.244 | 1.00 | 40.07 | C |
| ATOM | 5482 | O | MET | C | 171 | 53.794 | 17.072 | −13.701 | 1.00 | 32.95 | O |
| ATOM | 5483 | CB | MET | C | 171 | 51.082 | 18.018 | −14.611 | 1.00 | 41.02 | C |
| ATOM | 5484 | CG | MET | C | 171 | 49.660 | 17.462 | −14.718 | 1.00 | 38.96 | C |
| ATOM | 5485 | SD | MET | C | 171 | 49.048 | 17.511 | −16.430 | 1.00 | 52.12 | S |
| ATOM | 5486 | CE | MET | C | 171 | 49.795 | 16.031 | −17.113 | 1.00 | 40.51 | C |
| ATOM | 5487 | N | HIS | C | 172 | 53.813 | 19.138 | −12.793 | 1.00 | 38.07 | N |
| ATOM | 5488 | CA | HIS | C | 172 | 55.268 | 19.218 | −12.629 | 1.00 | 39.03 | C |
| ATOM | 5489 | C | HIS | C | 172 | 56.038 | 19.449 | −13.905 | 1.00 | 25.90 | C |
| ATOM | 5490 | O | HIS | C | 172 | 57.226 | 19.169 | −13.954 | 1.00 | 35.66 | O |
| ATOM | 5491 | CB | HIS | C | 172 | 55.830 | 17.970 | −11.939 | 1.00 | 40.31 | C |
| ATOM | 5492 | CG | HIS | C | 172 | 55.121 | 17.615 | −10.673 | 1.00 | 50.52 | C |
| ATOM | 5493 | ND1 | HIS | C | 172 | 55.208 | 18.385 | −9.534 | 1.00 | 50.52 | N |
| ATOM | 5494 | CD2 | HIS | C | 172 | 54.303 | 16.578 | −10.370 | 1.00 | 50.01 | C |
| ATOM | 5495 | CE1 | HIS | C | 172 | 54.477 | 17.834 | −8.579 | 1.00 | 54.33 | C |
| ATOM | 5496 | NE2 | HIS | C | 172 | 53.917 | 16.739 | −9.060 | 1.00 | 53.88 | N |
| ATOM | 5497 | N | TRP | C | 173 | 55.378 | 19.964 | −14.932 | 1.00 | 31.13 | N |
| ATOM | 5498 | CA | TRP | C | 173 | 56.030 | 20.180 | −16.223 | 1.00 | 29.61 | C |
| ATOM | 5499 | C | TRP | C | 173 | 56.991 | 21.370 | −16.184 | 1.00 | 35.21 | C |
| ATOM | 5500 | O | TRP | C | 173 | 57.774 | 21.577 | −17.110 | 1.00 | 34.17 | O |
| ATOM | 5501 | CB | TRP | C | 173 | 54.977 | 20.434 | −17.292 | 1.00 | 31.54 | C |
| ATOM | 5502 | CG | TRP | C | 173 | 54.112 | 19.255 | −17.634 | 1.00 | 31.81 | C |
| ATOM | 5503 | CD1 | TRP | C | 173 | 54.102 | 18.025 | −17.036 | 1.00 | 32.87 | C |
| ATOM | 5504 | CD2 | TRP | C | 173 | 53.096 | 19.221 | −18.639 | 1.00 | 27.89 | C |
| ATOM | 5505 | NE1 | TRP | C | 173 | 53.146 | 17.224 | −17.620 | 1.00 | 30.70 | N |
| ATOM | 5506 | CE2 | TRP | C | 173 | 52.519 | 17.937 | −18.609 | 1.00 | 34.12 | C |
| ATOM | 5507 | CE3 | TRP | C | 173 | 52.615 | 20.158 | −19.563 | 1.00 | 26.18 | C |
| ATOM | 5508 | CZ2 | TRP | C | 173 | 51.490 | 17.566 | −19.475 | 1.00 | 36.45 | C |
| ATOM | 5509 | CZ3 | TRP | C | 173 | 51.594 | 19.793 | −20.414 | 1.00 | 22.86 | C |
| ATOM | 5510 | CH2 | TRP | C | 173 | 51.049 | 18.505 | −20.372 | 1.00 | 28.04 | C |
| ATOM | 5511 | N | TRP | C | 174 | 56.913 | 22.154 | −15.114 | 1.00 | 28.74 | N |
| ATOM | 5512 | CA | TRP | C | 174 | 57.661 | 23.392 | −15.010 | 1.00 | 25.36 | C |
| ATOM | 5513 | C | TRP | C | 174 | 59.093 | 23.147 | −14.535 | 1.00 | 29.77 | C |
| ATOM | 5514 | O | TRP | C | 174 | 59.911 | 24.070 | −14.523 | 1.00 | 28.37 | O |
| ATOM | 5515 | CB | TRP | C | 174 | 56.944 | 24.353 | −14.047 | 1.00 | 31.67 | C |
| ATOM | 5516 | CG | TRP | C | 174 | 56.792 | 23.783 | −12.648 | 1.00 | 32.26 | C |
| ATOM | 5517 | CD1 | TRP | C | 174 | 55.859 | 22.879 | −12.233 | 1.00 | 30.07 | C |
| ATOM | 5518 | CD2 | TRP | C | 174 | 57.609 | 24.067 | −11.502 | 1.00 | 28.60 | C |
| ATOM | 5519 | NE1 | TRP | C | 174 | 56.039 | 22.583 | −10.904 | 1.00 | 33.83 | N |
| ATOM | 5520 | CE2 | TRP | C | 174 | 57.108 | 23.295 | −10.430 | 1.00 | 36.77 | C |
| ATOM | 5521 | CE3 | TRP | C | 174 | 58.710 | 24.898 | −11.276 | 1.00 | 31.63 | C |
| ATOM | 5522 | CZ2 | TRP | C | 174 | 57.671 | 23.332 | −9.148 | 1.00 | 32.27 | C |
| ATOM | 5523 | CZ3 | TRP | C | 174 | 59.264 | 24.934 | −10.000 | 1.00 | 36.95 | C |
| ATOM | 5524 | CH2 | TRP | C | 174 | 58.742 | 24.156 | −8.956 | 1.00 | 28.36 | C |
| ATOM | 5525 | N | ARG | C | 175 | 59.404 | 21.912 | −14.147 | 1.00 | 24.43 | N |
| ATOM | 5526 | CA | ARG | C | 175 | 60.688 | 21.658 | −13.482 | 1.00 | 35.43 | C |
| ATOM | 5527 | C | ARG | C | 175 | 61.900 | 21.544 | −14.408 | 1.00 | 32.95 | C |
| ATOM | 5528 | O | ARG | C | 175 | 61.814 | 21.012 | −15.515 | 1.00 | 29.79 | O |
| ATOM | 5529 | CB | ARG | C | 175 | 60.622 | 20.453 | −12.527 | 1.00 | 27.23 | C |
| ATOM | 5530 | CG | ARG | C | 175 | 59.531 | 20.569 | −11.481 | 1.00 | 33.16 | C |
| ATOM | 5531 | CD | ARG | C | 175 | 59.972 | 20.026 | −10.142 | 1.00 | 32.01 | C |
| ATOM | 5532 | NE | ARG | C | 175 | 59.074 | 18.987 | −9.651 | 1.00 | 47.03 | N |
| ATOM | 5533 | CZ | ARG | C | 175 | 58.687 | 18.864 | −8.384 | 1.00 | 62.54 | C |
| ATOM | 5534 | NH1 | ARG | C | 175 | 59.100 | 19.736 | −7.475 | 1.00 | 59.81 | N |
| ATOM | 5535 | NH2 | ARG | C | 175 | 57.867 | 17.881 | −8.030 | 1.00 | 66.33 | N |
| ATOM | 5536 | N | ASP | C | 176 | 63.026 | 22.052 | −13.916 | 1.00 | 30.02 | N |
| ATOM | 5537 | CA | ASP | C | 176 | 64.297 | 22.000 | −14.612 | 1.00 | 29.44 | C |
| ATOM | 5538 | C | ASP | C | 176 | 65.041 | 20.741 | −14.213 | 1.00 | 29.44 | C |
| ATOM | 5539 | O | ASP | C | 176 | 64.587 | 19.997 | −13.352 | 1.00 | 32.19 | O |
| ATOM | 5540 | CB | ASP | C | 176 | 65.127 | 23.229 | −14.269 | 1.00 | 40.94 | C |
| ATOM | 5541 | CG | ASP | C | 176 | 66.082 | 23.614 | −15.375 | 1.00 | 52.10 | C |
| ATOM | 5542 | OD1 | ASP | C | 176 | 66.438 | 22.730 | −16.181 | 1.00 | 50.29 | O |
| ATOM | 5543 | OD2 | ASP | C | 176 | 66.468 | 24.804 | −15.440 | 1.00 | 61.42 | O |
| ATOM | 5544 | N | GLU | C | 177 | 66.183 | 20.502 | −14.849 | 1.00 | 39.94 | N |
| ATOM | 5545 | CA | GLU | C | 177 | 66.919 | 19.253 | −14.671 | 1.00 | 46.66 | C |
| ATOM | 5546 | C | GLU | C | 177 | 68.215 | 19.520 | −13.914 | 1.00 | 50.47 | C |
| ATOM | 5547 | O | GLU | C | 177 | 68.776 | 18.636 | −13.251 | 1.00 | 32.00 | O |
| ATOM | 5548 | CB | GLU | C | 177 | 67.207 | 18.621 | −16.033 | 1.00 | 64.62 | C |
| ATOM | 5549 | CG | GLU | C | 177 | 67.285 | 17.116 | −15.974 | 1.00 | 89.42 | C |
| ATOM | 5550 | CD | GLU | C | 177 | 66.411 | 16.553 | −14.869 | 1.00 | 87.28 | C |
| ATOM | 5551 | OE1 | GLU | C | 177 | 65.218 | 16.925 | −14.812 | 1.00 | 85.94 | O |
| ATOM | 5552 | OE2 | GLU | C | 177 | 66.916 | 15.747 | −14.054 | 1.00 | 87.29 | O |
| ATOM | 5553 | N | ASP | C | 178 | 68.646 | 20.775 | −14.027 | 1.00 | 47.96 | N |
| ATOM | 5554 | CA | ASP | C | 178 | 69.811 | 21.346 | −13.358 | 1.00 | 44.10 | C |
| ATOM | 5555 | C | ASP | C | 178 | 69.959 | 20.975 | −11.889 | 1.00 | 45.88 | C |
| ATOM | 5556 | O | ASP | C | 178 | 68.976 | 20.928 | −11.151 | 1.00 | 53.65 | O |
| ATOM | 5557 | CB | ASP | C | 178 | 69.726 | 22.871 | −13.465 | 1.00 | 45.89 | C |
| ATOM | 5558 | CG | ASP | C | 178 | 71.016 | 23.560 | −13.069 | 1.00 | 64.23 | C |
| ATOM | 5559 | OD1 | ASP | C | 178 | 72.099 | 23.017 | −13.372 | 1.00 | 71.36 | O |
| ATOM | 5560 | OD2 | ASP | C | 178 | 70.951 | 24.653 | −12.467 | 1.00 | 68.05 | O |
| ATOM | 5561 | N | PRO | C | 179 | 71.205 | 20.725 | −11.457 | 1.00 | 54.24 | N |
| ATOM | 5562 | CA | PRO | C | 179 | 71.586 | 20.584 | −10.047 | 1.00 | 51.33 | C |
| ATOM | 5563 | C | PRO | C | 179 | 70.915 | 21.612 | −9.127 | 1.00 | 45.07 | C |
| ATOM | 5564 | O | PRO | C | 179 | 70.292 | 21.232 | −8.129 | 1.00 | 32.53 | O |
| ATOM | 5565 | CB | PRO | C | 179 | 73.095 | 20.834 | −10.087 | 1.00 | 46.19 | C |
| ATOM | 5566 | CG | PRO | C | 179 | 73.513 | 20.302 | −11.401 | 1.00 | 44.51 | C |
| ATOM | 5567 | CD | PRO | C | 179 | 72.347 | 20.467 | −12.353 | 1.00 | 46.52 | C |
| ATOM | 5568 | N | GLN | C | 180 | 71.057 | 22.895 | −9.451 | 1.00 | 36.91 | N |
| ATOM | 5569 | CA | GLN | C | 180 | 70.498 | 23.952 | −8.618 | 1.00 | 43.09 | C |
| ATOM | 5570 | C | GLN | C | 180 | 68.982 | 23.841 | −8.478 | 1.00 | 42.59 | C |
| ATOM | 5571 | O | GLN | C | 180 | 68.433 | 24.029 | −7.392 | 1.00 | 38.62 | O |
| ATOM | 5572 | CB | GLN | C | 180 | 70.882 | 25.328 | −9.153 | 1.00 | 51.52 | C |
| ATOM | 5573 | CG | GLN | C | 180 | 72.325 | 25.702 | −8.874 | 1.00 | 76.01 | C |
| ATOM | 5574 | CD | GLN | C | 180 | 72.645 | 27.133 | −9.259 | 1.00 | 108.26 | C |
| ATOM | 5575 | OE1 | GLN | C | 180 | 71.749 | 27.975 | −9.377 | 1.00 | 114.08 | O |
| ATOM | 5576 | NE2 | GLN | C | 180 | 73.929 | 27.419 | −9.455 | 1.00 | 116.20 | N |
| ATOM | 5577 | N | ALA | C | 181 | 68.310 | 23.532 | −9.579 | 1.00 | 36.06 | N |
| ATOM | 5578 | CA | ALA | C | 181 | 66.874 | 23.326 | −9.551 | 1.00 | 33.52 | C |
| ATOM | 5579 | C | ALA | C | 181 | 66.538 | 22.182 | −8.600 | 1.00 | 37.08 | C |
| ATOM | 5580 | O | ALA | C | 181 | 65.696 | 22.331 | −7.714 | 1.00 | 31.17 | O |
| ATOM | 5581 | CB | ALA | C | 181 | 66.348 | 23.040 | −10.946 | 1.00 | 34.36 | C |
| ATOM | 5582 | N | LEU | C | 182 | 67.206 | 21.044 | −8.775 | 1.00 | 36.19 | N |
| ATOM | 5583 | CA | LEU | C | 182 | 66.949 | 19.881 | −7.924 | 1.00 | 44.01 | C |
| ATOM | 5584 | C | LEU | C | 182 | 67.191 | 20.189 | −6.445 | 1.00 | 38.45 | C |
| ATOM | 5585 | O | LEU | C | 182 | 66.449 | 19.730 | −5.578 | 1.00 | 37.66 | O |
| ATOM | 5586 | CB | LEU | C | 182 | 67.781 | 18.679 | −8.369 | 1.00 | 47.13 | C |
| ATOM | 5587 | CG | LEU | C | 182 | 67.336 | 18.039 | −9.682 | 1.00 | 55.72 | C |
| ATOM | 5588 | CD1 | LEU | C | 182 | 68.080 | 16.734 | −9.909 | 1.00 | 58.51 | C |
| ATOM | 5589 | CD2 | LEU | C | 182 | 65.827 | 17.810 | −9.668 | 1.00 | 40.22 | C |
| ATOM | 5590 | N | LYS | C | 183 | 68.235 | 20.967 | −6.171 | 1.00 | 36.84 | N |
| ATOM | 5591 | CA | LYS | C | 183 | 68.513 | 21.440 | −4.820 | 1.00 | 38.02 | C |
| ATOM | 5592 | C | LYS | C | 183 | 67.312 | 22.165 | −4.245 | 1.00 | 37.82 | C |
| ATOM | 5593 | O | LYS | C | 183 | 66.953 | 21.958 | −3.090 | 1.00 | 32.61 | O |
| ATOM | 5594 | CB | LYS | C | 183 | 69.701 | 22.389 | −4.830 | 1.00 | 38.85 | C |
| ATOM | 5595 | CG | LYS | C | 183 | 70.248 | 22.682 | −3.467 | 1.00 | 42.96 | C |
| ATOM | 5596 | CD | LYS | C | 183 | 71.615 | 23.334 | −3.559 | 1.00 | 48.91 | C |
| ATOM | 5597 | CE | LYS | C | 183 | 72.159 | 23.638 | −2.173 | 1.00 | 61.15 | C |
| ATOM | 5598 | NZ | LYS | C | 183 | 73.271 | 24.623 | −2.229 | 1.00 | 79.11 | N |
| ATOM | 5599 | N | CYS | C | 184 | 66.693 | 23.013 | −5.063 | 1.00 | 36.90 | N |
| ATOM | 5600 | CA | CYS | C | 184 | 65.548 | 23.798 | −4.625 | 1.00 | 35.14 | C |
| ATOM | 5601 | C | CYS | C | 184 | 64.328 | 22.922 | −4.343 | 1.00 | 38.13 | C |
| ATOM | 5602 | O | CYS | C | 184 | 63.638 | 23.095 | −3.325 | 1.00 | 37.28 | O |
| ATOM | 5603 | CB | CYS | C | 184 | 65.197 | 24.867 | −5.660 | 1.00 | 33.65 | C |
| ATOM | 5604 | SG | CYS | C | 184 | 63.834 | 25.960 | −5.135 | 1.00 | 67.53 | S |
| ATOM | 5605 | N | TYR | C | 185 | 64.064 | 21.980 | −5.242 | 1.00 | 30.92 | N |
| ATOM | 5606 | CA | TYR | C | 185 | 62.882 | 21.143 | −5.119 | 1.00 | 32.04 | C |
| ATOM | 5607 | C | TYR | C | 185 | 62.885 | 20.307 | −3.844 | 1.00 | 33.53 | C |
| ATOM | 5608 | O | TYR | C | 185 | 61.839 | 19.843 | −3.391 | 1.00 | 35.47 | O |
| ATOM | 5609 | CB | TYR | C | 185 | 62.711 | 20.245 | −6.342 | 1.00 | 38.39 | C |
| ATOM | 5610 | CG | TYR | C | 185 | 62.687 | 20.998 | −7.645 | 1.00 | 35.05 | C |
| ATOM | 5611 | CD1 | TYR | C | 185 | 62.214 | 22.303 | −7.701 | 1.00 | 23.91 | C |
| ATOM | 5612 | CD2 | TYR | C | 185 | 63.142 | 20.401 | −8.825 | 1.00 | 28.95 | C |
| ATOM | 5613 | CE1 | TYR | C | 185 | 62.202 | 23.003 | −8.903 | 1.00 | 35.32 | C |
| ATOM | 5614 | CE2 | TYR | C | 185 | 63.136 | 21.088 | −10.032 | 1.00 | 26.73 | C |
| ATOM | 5615 | CZ | TYR | C | 185 | 62.659 | 22.387 | −10.067 | 1.00 | 31.32 | C |
| ATOM | 5616 | OH | TYR | C | 185 | 62.633 | 23.083 | −11.254 | 1.00 | 27.71 | O |
| ATOM | 5617 | N | GLN | C | 186 | 64.055 | 20.134 | −3.246 | 1.00 | 36.31 | N |
| ATOM | 5618 | CA | GLN | C | 186 | 64.146 | 19.314 | −2.046 | 1.00 | 45.11 | C |
| ATOM | 5619 | C | GLN | C | 186 | 64.266 | 20.113 | −0.756 | 1.00 | 37.94 | C |
| ATOM | 5620 | O | GLN | C | 186 | 64.208 | 19.556 | 0.340 | 1.00 | 42.51 | O |
| ATOM | 5621 | CB | GLN | C | 186 | 65.276 | 18.305 | −2.168 | 1.00 | 45.70 | C |
| ATOM | 5622 | CG | GLN | C | 186 | 64.867 | 17.079 | −2.938 | 1.00 | 45.66 | C |
| ATOM | 5623 | CD | GLN | C | 186 | 65.593 | 15.875 | −2.455 | 1.00 | 70.76 | C |
| ATOM | 5624 | OE1 | GLN | C | 186 | 66.211 | 15.903 | −1.388 | 1.00 | 91.13 | O |
| ATOM | 5625 | NE2 | GLN | C | 186 | 65.539 | 14.801 | −3.230 | 1.00 | 92.01 | N |
| ATOM | 5626 | N | ASP | C | 187 | 64.433 | 21.418 | −0.893 | 1.00 | 28.09 | N |
| ATOM | 5627 | CA | ASP | C | 187 | 64.446 | 22.285 | 0.263 | 1.00 | 37.87 | C |
| ATOM | 5628 | C | ASP | C | 187 | 63.039 | 22.845 | 0.455 | 1.00 | 33.83 | C |
| ATOM | 5629 | O | ASP | C | 187 | 62.585 | 23.644 | −0.372 | 1.00 | 35.60 | O |
| ATOM | 5630 | CB | ASP | C | 187 | 65.463 | 23.410 | 0.059 | 1.00 | 35.90 | C |
| ATOM | 5631 | CG | ASP | C | 187 | 65.545 | 24.366 | 1.255 | 1.00 | 49.41 | C |
| ATOM | 5632 | OD1 | ASP | C | 187 | 65.042 | 24.033 | 2.358 | 1.00 | 40.36 | O |
| ATOM | 5633 | OD2 | ASP | C | 187 | 66.122 | 25.465 | 1.081 | 1.00 | 60.45 | O |
| ATOM | 5634 | N | PRO | C | 188 | 62.337 | 22.404 | 1.527 | 1.00 | 23.88 | N |
| ATOM | 5635 | CA | PRO | C | 188 | 60.997 | 22.904 | 1.847 | 1.00 | 26.25 | C |
| ATOM | 5636 | C | PRO | C | 188 | 61.015 | 24.402 | 2.097 | 1.00 | 29.14 | C |
| ATOM | 5637 | O | PRO | C | 188 | 59.987 | 25.076 | 2.004 | 1.00 | 32.09 | O |
| ATOM | 5638 | CB | PRO | C | 188 | 60.628 | 22.144 | 3.117 | 1.00 | 17.62 | C |
| ATOM | 5639 | CG | PRO | C | 188 | 61.377 | 20.897 | 3.033 | 1.00 | 20.77 | C |
| ATOM | 5640 | CD | PRO | C | 188 | 62.684 | 21.245 | 2.361 | 1.00 | 29.00 | C |
| ATOM | 5641 | N | GLY | C | 189 | 62.195 | 24.923 | 2.387 | 1.00 | 28.73 | N |
| ATOM | 5642 | CA | GLY | C | 189 | 62.357 | 26.350 | 2.559 | 1.00 | 30.99 | C |
| ATOM | 5643 | C | GLY | C | 189 | 62.444 | 27.109 | 1.252 | 1.00 | 25.17 | C |
| ATOM | 5644 | O | GLY | C | 189 | 62.091 | 28.285 | 1.191 | 1.00 | 33.60 | O |
| ATOM | 5645 | N | CYS | C | 190 | 62.925 | 26.457 | 0.203 | 1.00 | 26.66 | N |
| ATOM | 5646 | CA | CYS | C | 190 | 63.029 | 27.134 | −1.090 | 1.00 | 42.07 | C |
| ATOM | 5647 | C | CYS | C | 190 | 61.710 | 27.073 | −1.879 | 1.00 | 39.35 | C |
| ATOM | 5648 | O | CYS | C | 190 | 61.181 | 25.983 | −2.144 | 1.00 | 38.96 | O |
| ATOM | 5649 | CB | CYS | C | 190 | 64.199 | 26.584 | −1.914 | 1.00 | 31.69 | C |
| ATOM | 5650 | SG | CYS | C | 190 | 64.300 | 27.254 | −3.599 | 1.00 | 51.78 | S |
| ATOM | 5651 | N | CYS | C | 191 | 61.179 | 28.249 | −2.215 | 1.00 | 31.71 | N |
| ATOM | 5652 | CA | CYS | C | 191 | 59.986 | 28.359 | −3.058 | 1.00 | 37.21 | C |
| ATOM | 5653 | C | CYS | C | 191 | 60.253 | 29.243 | −4.276 | 1.00 | 40.09 | C |
| ATOM | 5654 | O | CYS | C | 191 | 59.476 | 30.148 | −4.575 | 1.00 | 45.07 | O |
| ATOM | 5655 | CB | CYS | C | 191 | 58.775 | 28.896 | −2.269 | 1.00 | 28.12 | C |
| ATOM | 5656 | SG | CYS | C | 191 | 57.129 | 28.402 | −2.949 | 1.00 | 30.69 | S |
| ATOM | 5657 | N | ASP | C | 192 | 61.360 | 28.988 | −4.967 | 1.00 | 47.32 | N |
| ATOM | 5658 | CA | ASP | C | 192 | 61.677 | 29.701 | −6.200 | 1.00 | 36.52 | C |
| ATOM | 5659 | C | ASP | C | 192 | 60.978 | 29.019 | −7.336 | 1.00 | 37.88 | C |
| ATOM | 5660 | O | ASP | C | 192 | 61.017 | 27.791 | −7.456 | 1.00 | 44.51 | O |
| ATOM | 5661 | CB | ASP | C | 192 | 63.169 | 29.662 | −6.500 | 1.00 | 37.78 | C |
| ATOM | 5662 | CG | ASP | C | 192 | 63.985 | 30.443 | −5.512 | 1.00 | 56.08 | C |
| ATOM | 5663 | OD1 | ASP | C | 192 | 63.523 | 30.621 | −4.365 | 1.00 | 66.20 | O |
| ATOM | 5664 | OD2 | ASP | C | 192 | 65.097 | 30.871 | −5.887 | 1.00 | 73.78 | O |
| ATOM | 5665 | N | PHE | C | 193 | 60.345 | 29.809 | −8.186 | 1.00 | 38.40 | N |
| ATOM | 5666 | CA | PHE | C | 193 | 59.734 | 29.242 | −9.377 | 1.00 | 42.86 | C |
| ATOM | 5667 | C | PHE | C | 193 | 60.826 | 29.061 | −10.442 | 1.00 | 37.32 | C |
| ATOM | 5668 | O | PHE | C | 193 | 60.876 | 29.776 | −11.453 | 1.00 | 38.86 | O |
| ATOM | 5669 | CB | PHE | C | 193 | 58.562 | 30.106 | −9.858 | 1.00 | 30.64 | C |
| ATOM | 5670 | CG | PHE | C | 193 | 57.594 | 29.376 | −10.722 | 1.00 | 31.35 | C |
| ATOM | 5671 | CD1 | PHE | C | 193 | 57.341 | 28.024 | −10.505 | 1.00 | 27.69 | C |
| ATOM | 5672 | CD2 | PHE | C | 193 | 56.920 | 30.036 | −11.744 | 1.00 | 25.39 | C |
| ATOM | 5673 | CE1 | PHE | C | 193 | 56.433 | 27.337 | −11.298 | 1.00 | 23.99 | C |
| ATOM | 5674 | CE2 | PHE | C | 193 | 56.021 | 29.366 | −12.538 | 1.00 | 25.98 | C |
| ATOM | 5675 | CZ | PHE | C | 193 | 55.775 | 28.005 | −12.312 | 1.00 | 26.70 | C |
| ATOM | 5676 | N | VAL | C | 194 | 61.712 | 28.107 | −10.168 | 1.00 | 30.15 | N |
| ATOM | 5677 | CA | VAL | C | 194 | 62.812 | 27.748 | −11.053 | 1.00 | 31.15 | C |
| ATOM | 5678 | C | VAL | C | 194 | 62.300 | 26.786 | −12.116 | 1.00 | 31.06 | C |
| ATOM | 5679 | O | VAL | C | 194 | 62.051 | 25.608 | −11.831 | 1.00 | 29.20 | O |
| ATOM | 5680 | CB | VAL | C | 194 | 63.937 | 27.059 | −10.254 | 1.00 | 30.98 | C |
| ATOM | 5681 | CG1 | VAL | C | 194 | 65.036 | 26.558 | −11.182 | 1.00 | 29.95 | C |
| ATOM | 5682 | CG2 | VAL | C | 194 | 64.481 | 27.995 | −9.218 | 1.00 | 21.89 | C |
| ATOM | 5683 | N | THR | C | 195 | 62.143 | 27.277 | −13.341 | 1.00 | 28.88 | N |
| ATOM | 5684 | CA | THR | C | 195 | 61.483 | 26.479 | −14.374 | 1.00 | 31.24 | C |
| ATOM | 5685 | C | THR | C | 195 | 62.402 | 26.162 | −15.533 | 1.00 | 29.23 | C |
| ATOM | 5686 | O | THR | C | 195 | 63.382 | 26.863 | −15.763 | 1.00 | 41.30 | O |
| ATOM | 5687 | CB | THR | C | 195 | 60.205 | 27.172 | −14.899 | 1.00 | 32.41 | C |
| ATOM | 5688 | OG1 | THR | C | 195 | 60.541 | 28.442 | −15.472 | 1.00 | 31.47 | O |
| ATOM | 5689 | CG2 | THR | C | 195 | 59.218 | 27.403 | −13.759 | 1.00 | 35.99 | C |
| ATOM | 5690 | N | ASN | C | 196 | 62.090 | 25.096 | −16.258 | 1.00 | 30.93 | N |
| ATOM | 5691 | CA | ASN | C | 196 | 62.798 | 24.824 | −17.499 | 1.00 | 33.28 | C |
| ATOM | 5692 | C | ASN | C | 196 | 62.417 | 25.866 | −18.552 | 1.00 | 26.49 | C |
| ATOM | 5693 | O | ASN | C | 196 | 61.333 | 26.455 | −18.490 | 1.00 | 25.97 | O |
| ATOM | 5694 | CB | ASN | C | 196 | 62.548 | 23.390 | −17.976 | 1.00 | 30.77 | C |
| ATOM | 5695 | CG | ASN | C | 196 | 61.118 | 23.152 | −18.401 | 1.00 | 27.68 | C |
| ATOM | 5696 | OD1 | ASN | C | 196 | 60.604 | 23.810 | −19.298 | 1.00 | 30.67 | O |
| ATOM | 5697 | ND2 | ASN | C | 196 | 60.478 | 22.185 | −17.778 | 1.00 | 25.76 | N |
| ATOM | 5698 | N | ARG | C | 197 | 63.311 | 26.111 | −19.503 | 1.00 | 33.24 | N |
| ATOM | 5699 | CA | ARG | C | 197 | 63.108 | 27.204 | −20.464 | 1.00 | 37.94 | C |
| ATOM | 5700 | C | ARG | C | 197 | 61.921 | 26.994 | −21.415 | 1.00 | 28.30 | C |
| ATOM | 5701 | O | ARG | C | 197 | 61.290 | 27.958 | −21.859 | 1.00 | 27.80 | O |
| ATOM | 5702 | CB | ARG | C | 197 | 64.393 | 27.499 | −21.244 | 1.00 | 31.42 | C |
| ATOM | 5703 | CG | ARG | C | 197 | 65.524 | 28.031 | −20.372 | 1.00 | 46.04 | C |
| ATOM | 5704 | CD | ARG | C | 197 | 66.675 | 28.573 | −21.193 | 1.00 | 44.31 | C |
| ATOM | 5705 | NE | ARG | C | 197 | 67.804 | 28.957 | −20.351 | 1.00 | 63.52 | N |
| ATOM | 5706 | CZ | ARG | C | 197 | 68.931 | 29.494 | −20.809 | 1.00 | 88.38 | C |
| ATOM | 5707 | NH1 | ARG | C | 197 | 69.092 | 29.716 | −22.112 | 1.00 | 98.54 | N |
| ATOM | 5708 | NH2 | ARG | C | 197 | 69.900 | 29.813 | −19.964 | 1.00 | 86.98 | N |
| ATOM | 5709 | N | ALA | C | 198 | 61.617 | 25.740 | −21.724 | 1.00 | 19.46 | N |
| ATOM | 5710 | CA | ALA | C | 198 | 60.474 | 25.448 | −22.566 | 1.00 | 15.92 | C |
| ATOM | 5711 | C | ALA | C | 198 | 59.233 | 25.994 | −21.879 | 1.00 | 29.45 | C |
| ATOM | 5712 | O | ALA | C | 198 | 58.508 | 26.814 | −22.443 | 1.00 | 30.53 | O |
| ATOM | 5713 | CB | ALA | C | 198 | 60.346 | 23.961 | −22.796 | 1.00 | 21.39 | C |
| ATOM | 5714 | N | TYR | C | 199 | 59.013 | 25.562 | −20.640 | 1.00 | 31.13 | N |
| ATOM | 5715 | CA | TYR | C | 199 | 57.833 | 25.957 | −19.890 | 1.00 | 21.47 | C |
| ATOM | 5716 | C | TYR | C | 199 | 57.782 | 27.463 | −19.642 | 1.00 | 24.81 | C |
| ATOM | 5717 | O | TYR | C | 199 | 56.707 | 28.055 | −19.636 | 1.00 | 26.38 | O |
| ATOM | 5718 | CB | TYR | C | 199 | 57.734 | 25.182 | −18.575 | 1.00 | 24.21 | C |
| ATOM | 5719 | CG | TYR | C | 199 | 56.668 | 25.735 | −17.674 | 1.00 | 26.87 | C |
| ATOM | 5720 | CD1 | TYR | C | 199 | 55.439 | 25.103 | −17.540 | 1.00 | 30.48 | C |
| ATOM | 5721 | CD2 | TYR | C | 199 | 56.877 | 26.920 | −16.981 | 1.00 | 25.91 | C |
| ATOM | 5722 | CE1 | TYR | C | 199 | 54.453 | 25.634 | −16.717 | 1.00 | 27.59 | C |
| ATOM | 5723 | CE2 | TYR | C | 199 | 55.913 | 27.454 | −16.175 | 1.00 | 28.37 | C |
| ATOM | 5724 | CZ | TYR | C | 199 | 54.707 | 26.812 | −16.038 | 1.00 | 28.45 | C |
| ATOM | 5725 | OH | TYR | C | 199 | 53.764 | 27.378 | −15.215 | 1.00 | 35.19 | O |
| ATOM | 5726 | N | ALA | C | 200 | 58.936 | 28.091 | −19.442 | 1.00 | 31.16 | N |
| ATOM | 5727 | CA | ALA | C | 200 | 58.965 | 29.543 | −19.252 | 1.00 | 34.83 | C |
| ATOM | 5728 | C | ALA | C | 200 | 58.360 | 30.277 | −20.452 | 1.00 | 32.63 | C |
| ATOM | 5729 | O | ALA | C | 200 | 57.545 | 31.185 | −20.297 | 1.00 | 30.06 | O |
| ATOM | 5730 | CB | ALA | C | 200 | 60.367 | 30.026 | −18.994 | 1.00 | 23.20 | C |
| ATOM | 5731 | N | ILE | C | 201 | 58.753 | 29.879 | −21.653 | 1.00 | 28.67 | N |
| ATOM | 5732 | CA | ILE | C | 201 | 58.248 | 30.536 | −22.853 | 1.00 | 33.86 | C |
| ATOM | 5733 | C | ILE | C | 201 | 56.796 | 30.161 | −23.182 | 1.00 | 29.79 | C |
| ATOM | 5734 | O | ILE | C | 201 | 55.940 | 31.028 | −23.350 | 1.00 | 26.16 | O |
| ATOM | 5735 | CB | ILE | C | 201 | 59.157 | 30.245 | −24.054 | 1.00 | 32.01 | C |
| ATOM | 5736 | CG1 | ILE | C | 201 | 60.477 | 31.014 | −23.909 | 1.00 | 34.90 | C |
| ATOM | 5737 | CG2 | ILE | C | 201 | 58.457 | 30.640 | −25.335 | 1.00 | 27.43 | C |
| ATOM | 5738 | CD1 | ILE | C | 201 | 61.650 | 30.355 | −24.604 | 1.00 | 22.93 | C |
| ATOM | 5739 | N | ALA | C | 202 | 56.519 | 28.868 | −23.260 | 1.00 | 22.73 | N |
| ATOM | 5740 | CA | ALA | C | 202 | 55.185 | 28.415 | −23.592 | 1.00 | 21.85 | C |
| ATOM | 5741 | C | ALA | C | 202 | 54.145 | 29.114 | −22.734 | 1.00 | 29.37 | C |
| ATOM | 5742 | O | ALA | C | 202 | 53.169 | 29.661 | −23.250 | 1.00 | 29.11 | O |
| ATOM | 5743 | CB | ALA | C | 202 | 55.079 | 26.901 | −23.445 | 1.00 | 19.66 | C |
| ATOM | 5744 | N | SER | C | 203 | 54.373 | 29.111 | −21.423 | 1.00 | 30.30 | N |
| ATOM | 5745 | CA | SER | C | 203 | 53.371 | 29.580 | −20.461 | 1.00 | 29.60 | C |
| ATOM | 5746 | C | SER | C | 203 | 53.211 | 31.102 | −20.358 | 1.00 | 27.17 | C |
| ATOM | 5747 | O | SER | C | 203 | 52.124 | 31.573 | −20.044 | 1.00 | 23.95 | O |
| ATOM | 5748 | CB | SER | C | 203 | 53.614 | 28.978 | −19.078 | 1.00 | 24.83 | C |
| ATOM | 5749 | OG | SER | C | 203 | 54.656 | 29.657 | −18.419 | 1.00 | 34.61 | O |
| ATOM | 5750 | N | SER | C | 204 | 54.262 | 31.881 | −20.609 | 1.00 | 24.73 | N |
| ATOM | 5751 | CA | SER | C | 204 | 54.048 | 33.328 | −20.637 | 1.00 | 33.01 | C |
| ATOM | 5752 | C | SER | C | 204 | 53.302 | 33.748 | −21.905 | 1.00 | 32.93 | C |
| ATOM | 5753 | O | SER | C | 204 | 52.386 | 34.578 | −21.850 | 1.00 | 31.00 | O |
| ATOM | 5754 | CB | SER | C | 204 | 55.322 | 34.160 | −20.387 | 1.00 | 24.42 | C |
| ATOM | 5755 | OG | SER | C | 204 | 56.488 | 33.383 | −20.490 | 1.00 | 35.81 | O |
| ATOM | 5756 | N | ILE | C | 205 | 53.665 | 33.149 | −23.033 | 1.00 | 24.78 | N |
| ATOM | 5757 | CA | ILE | C | 205 | 52.935 | 33.386 | −24.268 | 1.00 | 27.32 | C |
| ATOM | 5758 | C | ILE | C | 205 | 51.458 | 33.019 | −24.134 | 1.00 | 30.61 | C |
| ATOM | 5759 | O | ILE | C | 205 | 50.577 | 33.789 | −24.498 | 1.00 | 34.02 | O |
| ATOM | 5760 | CB | ILE | C | 205 | 53.510 | 32.564 | −25.406 | 1.00 | 24.47 | C |
| ATOM | 5761 | CG1 | ILE | C | 205 | 54.884 | 33.113 | −25.798 | 1.00 | 28.65 | C |
| ATOM | 5762 | CG2 | ILE | C | 205 | 52.547 | 32.558 | −26.585 | 1.00 | 19.52 | C |
| ATOM | 5763 | CD1 | ILE | C | 205 | 55.715 | 32.133 | −26.627 | 1.00 | 24.39 | C |
| ATOM | 5764 | N | ILE | C | 206 | 51.202 | 31.837 | −23.592 | 1.00 | 29.27 | N |
| ATOM | 5765 | CA | ILE | C | 206 | 49.861 | 31.271 | −23.524 | 1.00 | 22.78 | C |
| ATOM | 5766 | C | ILE | C | 206 | 48.993 | 31.878 | −22.435 | 1.00 | 27.48 | C |
| ATOM | 5767 | O | ILE | C | 206 | 47.792 | 32.012 | −22.607 | 1.00 | 32.04 | O |
| ATOM | 5768 | CB | ILE | C | 206 | 49.937 | 29.754 | −23.290 | 1.00 | 22.98 | C |
| ATOM | 5769 | CG1 | ILE | C | 206 | 50.369 | 29.051 | −24.569 | 1.00 | 27.24 | C |
| ATOM | 5770 | CG2 | ILE | C | 206 | 48.614 | 29.214 | −22.802 | 1.00 | 24.96 | C |
| ATOM | 5771 | CD1 | ILE | C | 206 | 50.541 | 27.578 | −24.405 | 1.00 | 37.77 | C |
| ATOM | 5772 | N | SER | C | 207 | 49.600 | 32.236 | −21.306 | 1.00 | 37.22 | N |
| ATOM | 5773 | CA | SER | C | 207 | 48.848 | 32.766 | −20.166 | 1.00 | 30.09 | C |
| ATOM | 5774 | C | SER | C | 207 | 48.748 | 34.292 | −20.208 | 1.00 | 36.11 | C |
| ATOM | 5775 | O | SER | C | 207 | 47.855 | 34.876 | −19.589 | 1.00 | 34.82 | O |
| ATOM | 5776 | CB | SER | C | 207 | 49.470 | 32.315 | −18.838 | 1.00 | 24.50 | C |
| ATOM | 5777 | OG | SER | C | 207 | 49.502 | 30.901 | −18.708 | 1.00 | 27.16 | O |
| ATOM | 5778 | N | PHE | C | 208 | 49.658 | 34.933 | −20.944 | 1.00 | 30.50 | N |
| ATOM | 5779 | CA | PHE | C | 208 | 49.737 | 36.392 | −20.946 | 1.00 | 29.29 | C |
| ATOM | 5780 | C | PHE | C | 208 | 49.828 | 37.028 | −22.359 | 1.00 | 33.44 | C |
| ATOM | 5781 | O | PHE | C | 208 | 48.979 | 37.836 | −22.730 | 1.00 | 33.61 | O |
| ATOM | 5782 | CB | PHE | C | 208 | 50.889 | 36.845 | −20.037 | 1.00 | 26.34 | C |
| ATOM | 5783 | CG | PHE | C | 208 | 50.984 | 38.332 | −19.859 | 1.00 | 31.24 | C |
| ATOM | 5784 | CD1 | PHE | C | 208 | 50.208 | 38.984 | −18.914 | 1.00 | 28.16 | C |
| ATOM | 5785 | CD2 | PHE | C | 208 | 51.870 | 39.084 | −20.628 | 1.00 | 35.39 | C |
| ATOM | 5786 | CE1 | PHE | C | 208 | 50.308 | 40.358 | −18.741 | 1.00 | 28.80 | C |
| ATOM | 5787 | CE2 | PHE | C | 208 | 51.968 | 40.466 | −20.466 | 1.00 | 32.25 | C |
| ATOM | 5788 | CZ | PHE | C | 208 | 51.189 | 41.099 | −19.527 | 1.00 | 31.77 | C |
| ATOM | 5789 | N | TYR | C | 209 | 50.833 | 36.669 | −23.152 | 1.00 | 32.43 | N |
| ATOM | 5790 | CA | TYR | C | 209 | 51.063 | 37.371 | −24.425 | 1.00 | 35.84 | C |
| ATOM | 5791 | C | TYR | C | 209 | 49.910 | 37.339 | −25.410 | 1.00 | 35.04 | C |
| ATOM | 5792 | O | TYR | C | 209 | 49.412 | 38.387 | −25.820 | 1.00 | 39.66 | O |
| ATOM | 5793 | CB | TYR | C | 209 | 52.379 | 36.943 | −25.087 | 1.00 | 29.59 | C |
| ATOM | 5794 | CG | TYR | C | 209 | 53.529 | 37.549 | −24.343 | 1.00 | 41.44 | C |
| ATOM | 5795 | CD1 | TYR | C | 209 | 54.305 | 36.785 | −23.478 | 1.00 | 38.26 | C |
| ATOM | 5796 | CD2 | TYR | C | 209 | 53.781 | 38.911 | −24.428 | 1.00 | 36.52 | C |
| ATOM | 5797 | CE1 | TYR | C | 209 | 55.334 | 37.355 | −22.751 | 1.00 | 31.75 | C |
| ATOM | 5798 | CE2 | TYR | C | 209 | 54.805 | 39.490 | −23.707 | 1.00 | 43.61 | C |
| ATOM | 5799 | CZ | TYR | C | 209 | 55.577 | 38.709 | −22.870 | 1.00 | 38.87 | C |
| ATOM | 5800 | OH | TYR | C | 209 | 56.595 | 39.298 | −22.164 | 1.00 | 34.79 | O |
| ATOM | 5801 | N | ILE | C | 210 | 49.493 | 36.142 | −25.795 | 1.00 | 32.37 | N |
| ATOM | 5802 | CA | ILE | C | 210 | 48.319 | 35.978 | −26.645 | 1.00 | 30.16 | C |
| ATOM | 5803 | C | ILE | C | 210 | 47.089 | 36.740 | −26.143 | 1.00 | 36.22 | C |
| ATOM | 5804 | O | ILE | C | 210 | 46.544 | 37.582 | −26.866 | 1.00 | 37.50 | O |
| ATOM | 5805 | CB | ILE | C | 210 | 47.959 | 34.508 | −26.794 | 1.00 | 26.00 | C |
| ATOM | 5806 | CG1 | ILE | C | 210 | 48.964 | 33.828 | −27.719 | 1.00 | 26.31 | C |
| ATOM | 5807 | CG2 | ILE | C | 210 | 46.539 | 34.364 | −27.311 | 1.00 | 32.16 | C |
| ATOM | 5808 | CD1 | ILE | C | 210 | 48.899 | 32.327 | −27.661 | 1.00 | 31.77 | C |
| ATOM | 5809 | N | PRO | C | 211 | 46.642 | 36.450 | −24.904 | 1.00 | 34.80 | N |
| ATOM | 5810 | CA | PRO | C | 211 | 45.468 | 37.173 | −24.404 | 1.00 | 32.95 | C |
| ATOM | 5811 | C | PRO | C | 211 | 45.708 | 38.677 | −24.386 | 1.00 | 34.21 | C |
| ATOM | 5812 | O | PRO | C | 211 | 44.754 | 39.454 | −24.468 | 1.00 | 34.81 | O |
| ATOM | 5813 | CB | PRO | C | 211 | 45.308 | 36.650 | −22.972 | 1.00 | 23.44 | C |
| ATOM | 5814 | CG | PRO | C | 211 | 46.024 | 35.339 | −22.966 | 1.00 | 30.36 | C |
| ATOM | 5815 | CD | PRO | C | 211 | 47.160 | 35.487 | −23.915 | 1.00 | 31.02 | C |
| ATOM | 5816 | N | LEU | C | 212 | 46.968 | 39.086 | −24.281 | 1.00 | 28.26 | N |
| ATOM | 5817 | CA | LEU | C | 212 | 47.262 | 40.504 | −24.178 | 1.00 | 33.81 | C |
| ATOM | 5818 | C | LEU | C | 212 | 47.147 | 41.166 | −25.537 | 1.00 | 41.29 | C |
| ATOM | 5819 | O | LEU | C | 212 | 46.492 | 42.193 | −25.693 | 1.00 | 41.28 | O |
| ATOM | 5820 | CB | LEU | C | 212 | 48.653 | 40.742 | −23.625 | 1.00 | 31.92 | C |
| ATOM | 5821 | CG | LEU | C | 212 | 48.850 | 42.253 | −23.566 | 1.00 | 33.51 | C |
| ATOM | 5822 | CD1 | LEU | C | 212 | 48.232 | 42.764 | −22.293 | 1.00 | 33.76 | C |
| ATOM | 5823 | CD2 | LEU | C | 212 | 50.322 | 42.622 | −23.666 | 1.00 | 35.51 | C |
| ATOM | 5824 | N | LEU | C | 213 | 47.809 | 40.575 | −26.519 | 1.00 | 34.93 | N |
| ATOM | 5825 | CA | LEU | C | 213 | 47.684 | 41.042 | −27.878 | 1.00 | 34.75 | C |
| ATOM | 5826 | C | LEU | C | 213 | 46.208 | 41.169 | −28.246 | 1.00 | 38.02 | C |
| ATOM | 5827 | O | LEU | C | 213 | 45.738 | 42.270 | −28.518 | 1.00 | 38.36 | O |
| ATOM | 5828 | CB | LEU | C | 213 | 48.433 | 40.109 | −28.821 | 1.00 | 36.33 | C |
| ATOM | 5829 | CG | LEU | C | 213 | 49.895 | 40.063 | −28.373 | 1.00 | 45.19 | C |
| ATOM | 5830 | CD1 | LEU | C | 213 | 50.750 | 39.140 | −29.241 | 1.00 | 38.50 | C |
| ATOM | 5831 | CD2 | LEU | C | 213 | 50.470 | 41.477 | −28.341 | 1.00 | 36.87 | C |
| ATOM | 5832 | N | ILE | C | 214 | 45.473 | 40.058 | −28.215 | 1.00 | 34.84 | N |
| ATOM | 5833 | CA | ILE | C | 214 | 44.048 | 40.090 | −28.534 | 1.00 | 36.72 | C |
| ATOM | 5834 | C | ILE | C | 214 | 43.351 | 41.260 | −27.842 | 1.00 | 41.26 | C |
| ATOM | 5835 | O | ILE | C | 214 | 42.662 | 42.050 | −28.482 | 1.00 | 42.24 | O |
| ATOM | 5836 | CB | ILE | C | 214 | 43.331 | 38.786 | −28.146 | 1.00 | 30.17 | C |
| ATOM | 5837 | CG1 | ILE | C | 214 | 43.700 | 37.673 | −29.107 | 1.00 | 25.82 | C |
| ATOM | 5838 | CG2 | ILE | C | 214 | 41.823 | 38.963 | −28.188 | 1.00 | 24.22 | C |
| ATOM | 5839 | CD1 | ILE | C | 214 | 43.312 | 36.302 | −28.589 | 1.00 | 27.14 | C |
| ATOM | 5840 | N | MET | C | 215 | 43.533 | 41.382 | −26.535 | 1.00 | 35.35 | N |
| ATOM | 5841 | CA | MET | C | 215 | 42.855 | 42.445 | −25.817 | 1.00 | 38.41 | C |
| ATOM | 5842 | C | MET | C | 215 | 43.260 | 43.818 | −26.310 | 1.00 | 40.10 | C |
| ATOM | 5843 | O | MET | C | 215 | 42.438 | 44.716 | −26.379 | 1.00 | 41.92 | O |
| ATOM | 5844 | CB | MET | C | 215 | 43.122 | 42.376 | −24.325 | 1.00 | 46.52 | C |
| ATOM | 5845 | CG | MET | C | 215 | 42.452 | 43.525 | −23.587 | 1.00 | 53.39 | C |
| ATOM | 5846 | SD | MET | C | 215 | 43.138 | 43.840 | −21.962 | 1.00 | 55.64 | S |
| ATOM | 5847 | CE | MET | C | 215 | 44.790 | 44.371 | −22.399 | 1.00 | 37.25 | C |
| ATOM | 5848 | N | ILE | C | 216 | 44.534 | 43.985 | −26.633 | 1.00 | 48.12 | N |
| ATOM | 5849 | CA | ILE | C | 216 | 45.043 | 45.263 | −27.067 | 1.00 | 47.21 | C |
| ATOM | 5850 | C | ILE | C | 216 | 44.409 | 45.683 | −28.397 | 1.00 | 47.60 | C |
| ATOM | 5851 | O | ILE | C | 216 | 43.878 | 46.788 | −28.534 | 1.00 | 45.96 | O |
| ATOM | 5852 | CB | ILE | C | 216 | 46.598 | 45.283 | −27.153 | 1.00 | 52.91 | C |
| ATOM | 5853 | CG1 | ILE | C | 216 | 47.201 | 45.641 | −25.788 | 1.00 | 51.26 | C |
| ATOM | 5854 | CG2 | ILE | C | 216 | 47.093 | 46.250 | −28.222 | 1.00 | 44.85 | C |
| ATOM | 5855 | CD1 | ILE | C | 216 | 48.677 | 45.304 | −25.648 | 1.00 | 39.51 | C |
| ATOM | 5856 | N | PHE | C | 217 | 44.460 | 44.763 | −29.357 | 1.00 | 46.49 | N |
| ATOM | 5857 | CA | PHE | C | 217 | 43.843 | 44.922 | −30.670 | 1.00 | 46.01 | C |
| ATOM | 5858 | C | PHE | C | 217 | 42.351 | 45.248 | −30.541 | 1.00 | 50.54 | C |
| ATOM | 5859 | O | PHE | C | 217 | 41.921 | 46.374 | −30.797 | 1.00 | 52.61 | O |
| ATOM | 5860 | CB | PHE | C | 217 | 44.061 | 43.626 | −31.471 | 1.00 | 47.24 | C |
| ATOM | 5861 | CG | PHE | C | 217 | 43.406 | 43.602 | −32.836 | 1.00 | 71.74 | C |
| ATOM | 5862 | CD1 | PHE | C | 217 | 44.154 | 43.838 | −33.986 | 1.00 | 74.87 | C |
| ATOM | 5863 | CD2 | PHE | C | 217 | 42.054 | 43.297 | −32.976 | 1.00 | 66.24 | C |
| ATOM | 5864 | CE1 | PHE | C | 217 | 43.560 | 43.796 | −35.248 | 1.00 | 72.23 | C |
| ATOM | 5865 | CE2 | PHE | C | 217 | 41.455 | 43.256 | −34.235 | 1.00 | 62.21 | C |
| ATOM | 5866 | CZ | PHE | C | 217 | 42.211 | 43.505 | −35.370 | 1.00 | 62.00 | C |
| ATOM | 5867 | N | VAL | C | 218 | 41.570 | 44.257 | −30.126 | 1.00 | 45.10 | N |
| ATOM | 5868 | CA | VAL | C | 218 | 40.133 | 44.402 | −30.017 | 1.00 | 34.84 | C |
| ATOM | 5869 | C | VAL | C | 218 | 39.805 | 45.717 | −29.323 | 1.00 | 39.29 | C |
| ATOM | 5870 | O | VAL | C | 218 | 38.847 | 46.396 | −29.680 | 1.00 | 44.25 | O |
| ATOM | 5871 | CB | VAL | C | 218 | 39.514 | 43.186 | −29.279 | 1.00 | 38.50 | C |
| ATOM | 5872 | CG1 | VAL | C | 218 | 38.038 | 43.421 | −28.929 | 1.00 | 33.76 | C |
| ATOM | 5873 | CG2 | VAL | C | 218 | 39.685 | 41.922 | −30.113 | 1.00 | 27.73 | C |
| ATOM | 5874 | N | ALA | C | 219 | 40.624 | 46.089 | −28.349 | 1.00 | 36.78 | N |
| ATOM | 5875 | CA | ALA | C | 219 | 40.391 | 47.308 | −27.582 | 1.00 | 42.71 | C |
| ATOM | 5876 | C | ALA | C | 219 | 40.583 | 48.557 | −28.430 | 1.00 | 55.20 | C |
| ATOM | 5877 | O | ALA | C | 219 | 39.790 | 49.495 | −28.350 | 1.00 | 55.28 | O |
| ATOM | 5878 | CB | ALA | C | 219 | 41.298 | 47.360 | −26.367 | 1.00 | 42.30 | C |
| ATOM | 5879 | N | LEU | C | 220 | 41.642 | 48.578 | −29.232 | 1.00 | 49.23 | N |
| ATOM | 5880 | CA | LEU | C | 220 | 41.881 | 49.710 | −30.115 | 1.00 | 50.83 | C |
| ATOM | 5881 | C | LEU | C | 220 | 40.700 | 49.889 | −31.062 | 1.00 | 57.54 | C |
| ATOM | 5882 | O | LEU | C | 220 | 40.248 | 51.008 | −31.305 | 1.00 | 58.22 | O |
| ATOM | 5883 | CB | LEU | C | 220 | 43.180 | 49.524 | −30.894 | 1.00 | 48.29 | C |
| ATOM | 5884 | CG | LEU | C | 220 | 44.432 | 49.670 | −30.023 | 1.00 | 62.84 | C |
| ATOM | 5885 | CD1 | LEU | C | 220 | 45.682 | 49.253 | −30.775 | 1.00 | 48.37 | C |
| ATOM | 5886 | CD2 | LEU | C | 220 | 44.565 | 51.098 | −29.490 | 1.00 | 46.47 | C |
| ATOM | 5887 | N | ARG | C | 221 | 40.191 | 48.777 | −31.577 | 1.00 | 48.17 | N |
| ATOM | 5888 | CA | ARG | C | 221 | 39.042 | 48.814 | −32.467 | 1.00 | 52.10 | C |
| ATOM | 5889 | C | ARG | C | 221 | 37.866 | 49.535 | −31.812 | 1.00 | 57.31 | C |
| ATOM | 5890 | O | ARG | C | 221 | 37.283 | 50.451 | −32.386 | 1.00 | 55.56 | O |
| ATOM | 5891 | CB | ARG | C | 221 | 38.639 | 47.398 | −32.879 | 1.00 | 57.74 | C |
| ATOM | 5892 | CG | ARG | C | 221 | 39.610 | 46.739 | −33.846 | 1.00 | 61.91 | C |
| ATOM | 5893 | CD | ARG | C | 221 | 39.497 | 47.342 | −35.235 | 1.00 | 88.09 | C |
| ATOM | 5894 | NE | ARG | C | 221 | 40.552 | 46.876 | −36.134 | 1.00 | 112.26 | N |
| ATOM | 5895 | CZ | ARG | C | 221 | 40.603 | 47.157 | −37.434 | 1.00 | 125.08 | C |
| ATOM | 5896 | NH1 | ARG | C | 221 | 39.654 | 47.897 | −37.991 | 1.00 | 124.00 | N |
| ATOM | 5897 | NH2 | ARG | C | 221 | 41.602 | 46.697 | −38.179 | 1.00 | 118.41 | N |
| ATOM | 5898 | N | VAL | C | 222 | 37.521 | 49.119 | −30.601 | 1.00 | 61.88 | N |
| ATOM | 5899 | CA | VAL | C | 222 | 36.419 | 49.740 | −29.882 | 1.00 | 57.08 | C |
| ATOM | 5900 | C | VAL | C | 222 | 36.595 | 51.254 | −29.789 | 1.00 | 56.34 | C |
| ATOM | 5901 | O | VAL | C | 222 | 35.620 | 52.001 | −29.814 | 1.00 | 55.40 | O |
| ATOM | 5902 | CB | VAL | C | 222 | 36.268 | 49.147 | −28.481 | 1.00 | 42.21 | C |
| ATOM | 5903 | CG1 | VAL | C | 222 | 35.170 | 49.859 | −27.718 | 1.00 | 41.64 | C |
| ATOM | 5904 | CG2 | VAL | C | 222 | 35.973 | 47.664 | −28.583 | 1.00 | 45.93 | C |
| ATOM | 5905 | N | TYR | C | 223 | 37.840 | 51.705 | −29.695 | 1.00 | 61.23 | N |
| ATOM | 5906 | CA | TYR | C | 223 | 38.118 | 53.136 | −29.607 | 1.00 | 67.98 | C |
| ATOM | 5907 | C | TYR | C | 223 | 37.758 | 53.850 | −30.902 | 1.00 | 68.56 | C |
| ATOM | 5908 | O | TYR | C | 223 | 37.087 | 54.880 | −30.891 | 1.00 | 66.70 | O |
| ATOM | 5909 | CB | TYR | C | 223 | 39.586 | 53.398 | −29.274 | 1.00 | 62.43 | C |
| ATOM | 5910 | CG | TYR | C | 223 | 39.871 | 54.846 | −28.961 | 1.00 | 68.79 | C |
| ATOM | 5911 | CD1 | TYR | C | 223 | 39.330 | 55.449 | −27.833 | 1.00 | 74.33 | C |
| ATOM | 5912 | CD2 | TYR | C | 223 | 40.681 | 55.613 | −29.789 | 1.00 | 77.11 | C |
| ATOM | 5913 | CE1 | TYR | C | 223 | 39.586 | 56.775 | −27.535 | 1.00 | 87.02 | C |
| ATOM | 5914 | CE2 | TYR | C | 223 | 40.945 | 56.944 | −29.500 | 1.00 | 87.04 | C |
| ATOM | 5915 | CZ | TYR | C | 223 | 40.392 | 57.520 | −28.373 | 1.00 | 90.55 | C |
| ATOM | 5916 | OH | TYR | C | 223 | 40.650 | 58.840 | −28.081 | 1.00 | 93.20 | O |
| ATOM | 5917 | N | ARG | C | 224 | 38.216 | 53.303 | −32.020 | 1.00 | 65.33 | N |
| ATOM | 5918 | CA | ARG | C | 224 | 37.915 | 53.891 | −33.312 | 1.00 | 64.17 | C |
| ATOM | 5919 | C | ARG | C | 224 | 36.412 | 53.928 | −33.512 | 1.00 | 71.69 | C |
| ATOM | 5920 | O | ARG | C | 224 | 35.871 | 54.916 | −33.994 | 1.00 | 79.44 | O |
| ATOM | 5921 | CB | ARG | C | 224 | 38.613 | 53.119 | −34.433 | 1.00 | 62.48 | C |
| ATOM | 5922 | CG | ARG | C | 224 | 40.113 | 53.378 | −34.476 | 1.00 | 79.52 | C |
| ATOM | 5923 | CD | ARG | C | 224 | 40.890 | 52.319 | −35.248 | 1.00 | 88.95 | C |
| ATOM | 5924 | NE | ARG | C | 224 | 42.317 | 52.396 | −34.935 | 1.00 | 99.82 | N |
| ATOM | 5925 | CZ | ARG | C | 224 | 43.277 | 51.779 | −35.618 | 1.00 | 105.70 | C |
| ATOM | 5926 | NH1 | ARG | C | 224 | 42.972 | 51.033 | −36.670 | 1.00 | 105.31 | N |
| ATOM | 5927 | NH2 | ARG | C | 224 | 44.545 | 51.916 | −35.249 | 1.00 | 93.37 | N |
| ATOM | 5928 | N | GLU | C | 225 | 35.742 | 52.856 | −33.106 | 1.00 | 71.38 | N |
| ATOM | 5929 | CA | GLU | C | 225 | 34.296 | 52.758 | −33.239 | 1.00 | 71.79 | C |
| ATOM | 5930 | C | GLU | C | 225 | 33.565 | 53.802 | −32.418 | 1.00 | 71.27 | C |
| ATOM | 5931 | O | GLU | C | 225 | 32.563 | 54.357 | −32.857 | 1.00 | 84.13 | O |
| ATOM | 5932 | CB | GLU | C | 225 | 33.813 | 51.363 | −32.856 | 1.00 | 71.55 | C |
| ATOM | 5933 | CG | GLU | C | 225 | 33.838 | 50.395 | −34.014 | 1.00 | 84.21 | C |
| ATOM | 5934 | CD | GLU | C | 225 | 33.035 | 50.903 | −35.196 | 1.00 | 106.40 | C |
| ATOM | 5935 | OE1 | GLU | C | 225 | 32.124 | 51.739 | −34.982 | 1.00 | 107.57 | O |
| ATOM | 5936 | OE2 | GLU | C | 225 | 33.315 | 50.468 | −36.335 | 1.00 | 102.40 | O |
| ATOM | 5937 | N | ALA | C | 226 | 34.062 | 54.064 | −31.220 | 1.00 | 72.84 | N |
| ATOM | 5938 | CA | ALA | C | 226 | 33.430 | 55.048 | −30.356 | 1.00 | 83.74 | C |
| ATOM | 5939 | C | ALA | C | 226 | 33.597 | 56.441 | −30.954 | 1.00 | 89.71 | C |
| ATOM | 5940 | O | ALA | C | 226 | 32.710 | 57.286 | −30.833 | 1.00 | 89.70 | O |
| ATOM | 5941 | CB | ALA | C | 226 | 34.015 | 54.982 | −28.954 | 1.00 | 71.61 | C |
| ATOM | 5942 | N | LYS | C | 227 | 34.736 | 56.663 | −31.607 | 1.00 | 86.20 | N |
| ATOM | 5943 | CA | LYS | C | 227 | 35.017 | 57.928 | −32.279 | 1.00 | 79.31 | C |
| ATOM | 5944 | C | LYS | C | 227 | 34.138 | 58.112 | −33.515 | 1.00 | 90.45 | C |
| ATOM | 5945 | O | LYS | C | 227 | 33.530 | 59.169 | −33.698 | 1.00 | 89.07 | O |
| ATOM | 5946 | CB | LYS | C | 227 | 36.490 | 58.006 | −32.679 | 1.00 | 76.70 | C |
| ATOM | 5947 | CG | LYS | C | 227 | 37.417 | 58.489 | −31.581 | 1.00 | 86.70 | C |
| ATOM | 5948 | CD | LYS | C | 227 | 38.803 | 58.815 | −32.138 | 1.00 | 86.55 | C |
| ATOM | 5949 | CE | LYS | C | 227 | 39.623 | 59.631 | −31.144 | 1.00 | 101.09 | C |
| ATOM | 5950 | NZ | LYS | C | 227 | 40.945 | 60.047 | −31.698 | 1.00 | 92.85 | N |
| ATOM | 5951 | N | GLU | C | 228 | 34.077 | 57.081 | −34.358 | 1.00 | 90.18 | N |
| ATOM | 5952 | CA | GLU | C | 228 | 33.220 | 57.086 | −35.548 | 1.00 | 88.21 | C |
| ATOM | 5953 | C | GLU | C | 228 | 31.756 | 57.252 | −35.174 | 1.00 | 82.11 | C |
| ATOM | 5954 | O | GLU | C | 228 | 30.889 | 57.266 | −36.041 | 1.00 | 93.94 | O |
| ATOM | 5955 | CB | GLU | C | 228 | 33.369 | 55.787 | −36.347 | 1.00 | 90.73 | C |
| ATOM | 5956 | CG | GLU | C | 228 | 34.662 | 55.646 | −37.131 | 1.00 | 107.96 | C |
| ATOM | 5957 | CD | GLU | C | 228 | 34.774 | 54.293 | −37.824 | 1.00 | 119.01 | C |
| ATOM | 5958 | OE1 | GLU | C | 228 | 33.820 | 53.901 | −38.532 | 1.00 | 103.70 | O |
| ATOM | 5959 | OE2 | GLU | C | 228 | 35.818 | 53.623 | −37.661 | 1.00 | 121.96 | O |
| ATOM | 5960 | N | GLN | C | 229 | 31.483 | 57.347 | −33.879 | 1.00 | 94.36 | N |
| ATOM | 5961 | CA | GLN | C | 229 | 30.129 | 57.577 | −33.403 | 1.00 | 90.87 | C |
| ATOM | 5962 | C | GLN | C | 229 | 29.949 | 59.025 | −32.979 | 1.00 | 88.63 | C |
| ATOM | 5963 | O | GLN | C | 229 | 28.853 | 59.573 | −33.088 | 1.00 | 97.12 | O |
| ATOM | 5964 | CB | GLN | C | 229 | 29.793 | 56.642 | −32.237 | 1.00 | 86.76 | C |
| ATOM | 5965 | CG | GLN | C | 229 | 29.355 | 55.250 | −32.666 | 1.00 | 89.40 | C |
| ATOM | 5966 | CD | GLN | C | 229 | 28.831 | 54.421 | −31.509 | 1.00 | 98.76 | C |
| ATOM | 5967 | OE1 | GLN | C | 229 | 28.113 | 53.440 | −31.709 | 1.00 | 92.31 | O |
| ATOM | 5968 | NE2 | GLN | C | 229 | 29.187 | 54.812 | −30.289 | 1.00 | 99.18 | N |
| ATOM | 5969 | N | ILE | C | 230 | 31.034 | 59.640 | −32.516 | 1.00 | 80.71 | N |
| ATOM | 5970 | CA | ILE | C | 230 | 30.984 | 60.947 | −31.872 | 1.00 | 101.22 | C |
| ATOM | 5971 | C | ILE | C | 230 | 31.932 | 60.924 | −30.700 | 1.00 | 112.02 | C |
| ATOM | 5972 | O | ILE | C | 230 | 33.142 | 61.110 | −30.826 | 1.00 | 116.06 | O |
| ATOM | 5973 | CB | ILE | C | 230 | 29.636 | 61.186 | −31.186 | 1.00 | 118.06 | C |
| ATOM | 5974 | CG1 | ILE | C | 230 | 29.711 | 62.438 | −30.317 | 1.00 | 116.29 | C |
| ATOM | 5975 | CG2 | ILE | C | 230 | 29.299 | 60.019 | −30.255 | 1.00 | 114.50 | C |
| ATOM | 5976 | CD1 | ILE | C | 230 | 29.230 | 62.205 | −28.891 | 1.00 | 114.01 | C |
| ATOM | 5977 | N | ARG | C | 267 | 27.114 | 59.218 | −21.807 | 1.00 | 99.65 | N |
| ATOM | 5978 | CA | ARG | C | 267 | 27.833 | 58.880 | −20.590 | 1.00 | 98.46 | C |
| ATOM | 5979 | C | ARG | C | 267 | 28.524 | 57.547 | −20.806 | 1.00 | 103.69 | C |
| ATOM | 5980 | O | ARG | C | 267 | 29.478 | 57.210 | −20.108 | 1.00 | 94.85 | O |
| ATOM | 5981 | CB | ARG | C | 267 | 26.862 | 58.752 | −19.413 | 1.00 | 112.20 | C |
| ATOM | 5982 | CG | ARG | C | 267 | 26.305 | 60.070 | −18.893 | 1.00 | 135.87 | C |
| ATOM | 5983 | CD | ARG | C | 267 | 27.291 | 60.785 | −17.965 | 1.00 | 142.64 | C |
| ATOM | 5984 | NE | ARG | C | 267 | 26.876 | 62.159 | −17.678 | 1.00 | 155.72 | N |
| ATOM | 5985 | CZ | ARG | C | 267 | 27.579 | 63.026 | −16.953 | 1.00 | 140.45 | C |
| ATOM | 5986 | NH1 | ARG | C | 267 | 28.744 | 62.668 | −16.427 | 1.00 | 134.81 | N |
| ATOM | 5987 | NH2 | ARG | C | 267 | 27.115 | 64.256 | −16.751 | 1.00 | 113.67 | N |
| ATOM | 5988 | N | GLU | C | 268 | 28.025 | 56.794 | −21.784 | 1.00 | 114.18 | N |
| ATOM | 5989 | CA | GLU | C | 268 | 28.515 | 55.449 | −22.084 | 1.00 | 101.00 | C |
| ATOM | 5990 | C | GLU | C | 268 | 30.014 | 55.429 | −22.341 | 1.00 | 91.48 | C |
| ATOM | 5991 | O | GLU | C | 268 | 30.678 | 54.409 | −22.150 | 1.00 | 83.95 | O |
| ATOM | 5992 | CB | GLU | C | 268 | 27.784 | 54.875 | −23.300 | 1.00 | 105.34 | C |
| ATOM | 5993 | CG | GLU | C | 268 | 26.265 | 54.967 | −23.221 | 1.00 | 124.17 | C |
| ATOM | 5994 | CD | GLU | C | 268 | 25.677 | 54.116 | −22.106 | 1.00 | 136.24 | C |
| ATOM | 5995 | OE1 | GLU | C | 268 | 26.426 | 53.315 | −21.507 | 1.00 | 135.19 | O |
| ATOM | 5996 | OE2 | GLU | C | 268 | 24.464 | 54.249 | −21.833 | 1.00 | 120.57 | O |
| ATOM | 5997 | N | HIS | C | 269 | 30.546 | 56.561 | −22.780 | 1.00 | 85.84 | N |
| ATOM | 5998 | CA | HIS | C | 269 | 31.967 | 56.659 | −23.071 | 1.00 | 86.16 | C |
| ATOM | 5999 | C | HIS | C | 269 | 32.824 | 56.707 | −21.808 | 1.00 | 81.90 | C |
| ATOM | 6000 | O | HIS | C | 269 | 33.910 | 56.128 | −21.761 | 1.00 | 69.05 | O |
| ATOM | 6001 | CB | HIS | C | 269 | 32.227 | 57.863 | −23.966 | 1.00 | 90.42 | C |
| ATOM | 6002 | CG | HIS | C | 269 | 31.768 | 57.658 | −25.373 | 1.00 | 100.75 | C |
| ATOM | 6003 | ND1 | HIS | C | 269 | 32.549 | 57.969 | −26.465 | 1.00 | 102.38 | N |
| ATOM | 6004 | CD2 | HIS | C | 269 | 30.619 | 57.139 | −25.866 | 1.00 | 105.46 | C |
| ATOM | 6005 | CE1 | HIS | C | 269 | 31.892 | 57.669 | −27.571 | 1.00 | 103.19 | C |
| ATOM | 6006 | NE2 | HIS | C | 269 | 30.719 | 57.163 | −27.237 | 1.00 | 106.74 | N |
| ATOM | 6007 | N | LYS | C | 270 | 32.335 | 57.398 | −20.785 | 1.00 | 84.94 | N |
| ATOM | 6008 | CA | LYS | C | 270 | 33.032 | 57.416 | −19.509 | 1.00 | 84.11 | C |
| ATOM | 6009 | C | LYS | C | 270 | 33.159 | 55.982 | −19.017 | 1.00 | 74.02 | C |
| ATOM | 6010 | O | LYS | C | 270 | 34.207 | 55.578 | −18.520 | 1.00 | 66.77 | O |
| ATOM | 6011 | CB | LYS | C | 270 | 32.293 | 58.292 | −18.494 | 1.00 | 85.75 | C |
| ATOM | 6012 | CG | LYS | C | 270 | 32.225 | 59.756 | −18.909 | 1.00 | 109.03 | C |
| ATOM | 6013 | CD | LYS | C | 270 | 31.214 | 60.548 | −18.088 | 1.00 | 118.93 | C |
| ATOM | 6014 | CE | LYS | C | 270 | 30.969 | 61.921 | −18.702 | 1.00 | 115.36 | C |
| ATOM | 6015 | NZ | LYS | C | 270 | 32.249 | 62.625 | −19.018 | 1.00 | 117.95 | N |
| ATOM | 6016 | N | ALA | C | 271 | 32.086 | 55.212 | −19.182 | 1.00 | 76.50 | N |
| ATOM | 6017 | CA | ALA | C | 271 | 32.092 | 53.799 | −18.819 | 1.00 | 66.29 | C |
| ATOM | 6018 | C | ALA | C | 271 | 33.120 | 53.057 | −19.659 | 1.00 | 64.83 | C |
| ATOM | 6019 | O | ALA | C | 271 | 33.917 | 52.280 | −19.132 | 1.00 | 54.11 | O |
| ATOM | 6020 | CB | ALA | C | 271 | 30.709 | 53.183 | −18.995 | 1.00 | 52.24 | C |
| ATOM | 6021 | N | LEU | C | 272 | 33.112 | 53.301 | −20.965 | 1.00 | 58.84 | N |
| ATOM | 6022 | CA | LEU | C | 272 | 34.102 | 52.677 | −21.820 | 1.00 | 63.60 | C |
| ATOM | 6023 | C | LEU | C | 272 | 35.496 | 53.097 | −21.387 | 1.00 | 56.10 | C |
| ATOM | 6024 | O | LEU | C | 272 | 36.380 | 52.255 | −21.231 | 1.00 | 54.77 | O |
| ATOM | 6025 | CB | LEU | C | 272 | 33.865 | 53.011 | −23.295 | 1.00 | 74.65 | C |
| ATOM | 6026 | CG | LEU | C | 272 | 32.661 | 52.337 | −23.961 | 1.00 | 70.84 | C |
| ATOM | 6027 | CD1 | LEU | C | 272 | 32.768 | 52.475 | −25.467 | 1.00 | 56.81 | C |
| ATOM | 6028 | CD2 | LEU | C | 272 | 32.553 | 50.871 | −23.564 | 1.00 | 52.22 | C |
| ATOM | 6029 | N | LYS | C | 273 | 35.684 | 54.395 | −21.169 | 1.00 | 58.98 | N |
| ATOM | 6030 | CA | LYS | C | 273 | 37.003 | 54.909 | −20.818 | 1.00 | 60.29 | C |
| ATOM | 6031 | C | LYS | C | 273 | 37.533 | 54.254 | −19.547 | 1.00 | 56.92 | C |
| ATOM | 6032 | O | LYS | C | 273 | 38.697 | 53.859 | −19.486 | 1.00 | 54.15 | O |
| ATOM | 6033 | CB | LYS | C | 273 | 36.995 | 56.430 | −20.665 | 1.00 | 65.43 | C |
| ATOM | 6034 | CG | LYS | C | 273 | 38.393 | 57.029 | −20.758 | 1.00 | 67.27 | C |
| ATOM | 6035 | CD | LYS | C | 273 | 38.513 | 58.349 | −20.021 | 1.00 | 67.50 | C |
| ATOM | 6036 | CE | LYS | C | 273 | 39.931 | 58.899 | −20.147 | 1.00 | 75.82 | C |
| ATOM | 6037 | NZ | LYS | C | 273 | 40.176 | 60.073 | −19.254 | 1.00 | 91.27 | N |
| ATOM | 6038 | N | THR | C | 274 | 36.678 | 54.143 | −18.534 | 1.00 | 52.35 | N |
| ATOM | 6039 | CA | THR | C | 274 | 37.051 | 53.457 | −17.303 | 1.00 | 57.60 | C |
| ATOM | 6040 | C | THR | C | 274 | 37.596 | 52.063 | −17.608 | 1.00 | 51.69 | C |
| ATOM | 6041 | O | THR | C | 274 | 38.625 | 51.670 | −17.066 | 1.00 | 42.03 | O |
| ATOM | 6042 | CB | THR | C | 274 | 35.863 | 53.336 | −16.320 | 1.00 | 62.31 | C |
| ATOM | 6043 | OG1 | THR | C | 274 | 35.598 | 54.610 | −15.713 | 1.00 | 68.90 | O |
| ATOM | 6044 | CG2 | THR | C | 274 | 36.177 | 52.327 | −15.226 | 1.00 | 53.15 | C |
| ATOM | 6045 | N | LEU | C | 275 | 36.911 | 51.329 | −18.485 | 1.00 | 49.01 | N |
| ATOM | 6046 | CA | LEU | C | 275 | 37.328 | 49.978 | −18.845 | 1.00 | 40.47 | C |
| ATOM | 6047 | C | LEU | C | 275 | 38.696 | 49.979 | −19.500 | 1.00 | 42.43 | C |
| ATOM | 6048 | O | LEU | C | 275 | 39.504 | 49.090 | −19.252 | 1.00 | 50.74 | O |
| ATOM | 6049 | CB | LEU | C | 275 | 36.312 | 49.308 | −19.765 | 1.00 | 30.69 | C |
| ATOM | 6050 | CG | LEU | C | 275 | 34.905 | 49.171 | −19.191 | 1.00 | 42.31 | C |
| ATOM | 6051 | CD1 | LEU | C | 275 | 33.971 | 48.475 | −20.171 | 1.00 | 30.51 | C |
| ATOM | 6052 | CD2 | LEU | C | 275 | 34.938 | 48.447 | −17.854 | 1.00 | 37.83 | C |
| ATOM | 6053 | N | GLY | C | 276 | 38.960 | 50.973 | −20.336 | 1.00 | 40.24 | N |
| ATOM | 6054 | CA | GLY | C | 276 | 40.251 | 51.078 | −20.989 | 1.00 | 36.24 | C |
| ATOM | 6055 | C | GLY | C | 276 | 41.340 | 51.364 | −19.978 | 1.00 | 41.04 | C |
| ATOM | 6056 | O | GLY | C | 276 | 42.476 | 50.938 | −20.138 | 1.00 | 46.37 | O |
| ATOM | 6057 | N | ILE | C | 277 | 40.986 | 52.087 | −18.925 | 1.00 | 40.15 | N |
| ATOM | 6058 | CA | ILE | C | 277 | 41.931 | 52.394 | −17.864 | 1.00 | 44.60 | C |
| ATOM | 6059 | C | ILE | C | 277 | 42.230 | 51.152 | −17.011 | 1.00 | 46.43 | C |
| ATOM | 6060 | O | ILE | C | 277 | 43.389 | 50.873 | −16.695 | 1.00 | 47.86 | O |
| ATOM | 6061 | CB | ILE | C | 277 | 41.425 | 53.558 | −16.995 | 1.00 | 49.65 | C |
| ATOM | 6062 | CG1 | ILE | C | 277 | 41.402 | 54.850 | −17.816 | 1.00 | 42.71 | C |
| ATOM | 6063 | CG2 | ILE | C | 277 | 42.295 | 53.726 | −15.753 | 1.00 | 37.10 | C |
| ATOM | 6064 | CD1 | ILE | C | 277 | 40.687 | 55.996 | −17.136 | 1.00 | 46.73 | C |
| ATOM | 6065 | N | ILE | C | 278 | 41.182 | 50.418 | −16.641 | 1.00 | 41.13 | N |
| ATOM | 6066 | CA | ILE | C | 278 | 41.320 | 49.106 | −16.015 | 1.00 | 36.15 | C |
| ATOM | 6067 | C | ILE | C | 278 | 42.344 | 48.271 | −16.781 | 1.00 | 45.83 | C |
| ATOM | 6068 | O | ILE | C | 278 | 43.257 | 47.669 | −16.189 | 1.00 | 39.16 | O |
| ATOM | 6069 | CB | ILE | C | 278 | 39.975 | 48.343 | −16.031 | 1.00 | 35.12 | C |
| ATOM | 6070 | CG1 | ILE | C | 278 | 38.957 | 49.018 | −15.119 | 1.00 | 42.49 | C |
| ATOM | 6071 | CG2 | ILE | C | 278 | 40.151 | 46.900 | −15.627 | 1.00 | 38.87 | C |
| ATOM | 6072 | CD1 | ILE | C | 278 | 39.539 | 49.551 | −13.853 | 1.00 | 50.28 | C |
| ATOM | 6073 | N | MET | C | 279 | 42.190 | 48.258 | −18.103 | 1.00 | 36.04 | N |
| ATOM | 6074 | CA | MET | C | 279 | 43.045 | 47.468 | −18.986 | 1.00 | 42.61 | C |
| ATOM | 6075 | C | MET | C | 279 | 44.471 | 48.001 | −19.081 | 1.00 | 40.50 | C |
| ATOM | 6076 | O | MET | C | 279 | 45.422 | 47.230 | −19.022 | 1.00 | 47.57 | O |
| ATOM | 6077 | CB | MET | C | 279 | 42.444 | 47.375 | −20.396 | 1.00 | 40.96 | C |
| ATOM | 6078 | CG | MET | C | 279 | 41.132 | 46.606 | −20.485 | 1.00 | 41.77 | C |
| ATOM | 6079 | SD | MET | C | 279 | 40.450 | 46.627 | −22.161 | 1.00 | 67.30 | S |
| ATOM | 6080 | CE | MET | C | 279 | 38.736 | 46.183 | −21.854 | 1.00 | 41.57 | C |
| ATOM | 6081 | N | GLY | C | 280 | 44.620 | 49.313 | −19.234 | 1.00 | 36.86 | N |
| ATOM | 6082 | CA | GLY | C | 280 | 45.933 | 49.908 | −19.421 | 1.00 | 39.92 | C |
| ATOM | 6083 | C | GLY | C | 280 | 46.789 | 49.807 | −18.173 | 1.00 | 45.26 | C |
| ATOM | 6084 | O | GLY | C | 280 | 47.995 | 49.534 | −18.232 | 1.00 | 34.76 | O |
| ATOM | 6085 | N | VAL | C | 281 | 46.151 | 50.034 | −17.031 | 1.00 | 36.28 | N |
| ATOM | 6086 | CA | VAL | C | 281 | 46.825 | 49.874 | −15.762 | 1.00 | 37.13 | C |
| ATOM | 6087 | C | VAL | C | 281 | 47.224 | 48.417 | −15.590 | 1.00 | 37.96 | C |
| ATOM | 6088 | O | VAL | C | 281 | 48.288 | 48.126 | −15.060 | 1.00 | 39.33 | O |
| ATOM | 6089 | CB | VAL | C | 281 | 45.947 | 50.328 | −14.572 | 1.00 | 46.83 | C |
| ATOM | 6090 | CG1 | VAL | C | 281 | 46.469 | 49.748 | −13.266 | 1.00 | 37.96 | C |
| ATOM | 6091 | CG2 | VAL | C | 281 | 45.889 | 51.842 | −14.494 | 1.00 | 41.78 | C |
| ATOM | 6092 | N | PHE | C | 282 | 46.379 | 47.495 | −16.036 | 1.00 | 34.25 | N |
| ATOM | 6093 | CA | PHE | C | 282 | 46.737 | 46.088 | −15.913 | 1.00 | 30.73 | C |
| ATOM | 6094 | C | PHE | C | 282 | 48.018 | 45.806 | −16.685 | 1.00 | 43.33 | C |
| ATOM | 6095 | O | PHE | C | 282 | 48.936 | 45.147 | −16.181 | 1.00 | 44.86 | O |
| ATOM | 6096 | CB | PHE | C | 282 | 45.632 | 45.165 | −16.413 | 1.00 | 29.27 | C |
| ATOM | 6097 | CG | PHE | C | 282 | 45.992 | 43.708 | −16.332 | 1.00 | 34.08 | C |
| ATOM | 6098 | CD1 | PHE | C | 282 | 45.641 | 42.953 | −15.223 | 1.00 | 36.94 | C |
| ATOM | 6099 | CD2 | PHE | C | 282 | 46.709 | 43.093 | −17.355 | 1.00 | 36.74 | C |
| ATOM | 6100 | CE1 | PHE | C | 282 | 45.982 | 41.609 | −15.139 | 1.00 | 26.58 | C |
| ATOM | 6101 | CE2 | PHE | C | 282 | 47.059 | 41.753 | −17.272 | 1.00 | 28.78 | C |
| ATOM | 6102 | CZ | PHE | C | 282 | 46.695 | 41.015 | −16.165 | 1.00 | 23.99 | C |
| ATOM | 6103 | N | THR | C | 283 | 48.078 | 46.310 | −17.912 | 1.00 | 34.43 | N |
| ATOM | 6104 | CA | THR | C | 283 | 49.193 | 46.021 | −18.788 | 1.00 | 32.81 | C |
| ATOM | 6105 | C | THR | C | 283 | 50.463 | 46.641 | −18.241 | 1.00 | 40.24 | C |
| ATOM | 6106 | O | THR | C | 283 | 51.537 | 46.049 | −18.334 | 1.00 | 43.70 | O |
| ATOM | 6107 | CB | THR | C | 283 | 48.932 | 46.536 | −20.216 | 1.00 | 39.34 | C |
| ATOM | 6108 | OG1 | THR | C | 283 | 47.731 | 45.942 | −20.716 | 1.00 | 44.17 | O |
| ATOM | 6109 | CG2 | THR | C | 283 | 50.095 | 46.186 | −21.158 | 1.00 | 29.48 | C |
| ATOM | 6110 | N | LEU | C | 284 | 50.337 | 47.837 | −17.676 | 1.00 | 42.21 | N |
| ATOM | 6111 | CA | LEU | C | 284 | 51.490 | 48.535 | −17.125 | 1.00 | 46.01 | C |
| ATOM | 6112 | C | LEU | C | 284 | 52.035 | 47.798 | −15.910 | 1.00 | 43.39 | C |
| ATOM | 6113 | O | LEU | C | 284 | 53.244 | 47.643 | −15.763 | 1.00 | 45.88 | O |
| ATOM | 6114 | CB | LEU | C | 284 | 51.113 | 49.972 | −16.761 | 1.00 | 54.66 | C |
| ATOM | 6115 | CG | LEU | C | 284 | 51.207 | 50.954 | −17.927 | 1.00 | 62.95 | C |
| ATOM | 6116 | CD1 | LEU | C | 284 | 50.289 | 52.168 | −17.733 | 1.00 | 46.70 | C |
| ATOM | 6117 | CD2 | LEU | C | 284 | 52.661 | 51.362 | −18.110 | 1.00 | 54.16 | C |
| ATOM | 6118 | N | CYS | C | 285 | 51.129 | 47.328 | −15.058 | 1.00 | 34.31 | N |
| ATOM | 6119 | CA | CYS | C | 285 | 51.496 | 46.664 | −13.811 | 1.00 | 34.98 | C |
| ATOM | 6120 | C | CYS | C | 285 | 52.076 | 45.264 | −13.985 | 1.00 | 37.69 | C |
| ATOM | 6121 | O | CYS | C | 285 | 52.842 | 44.807 | −13.143 | 1.00 | 44.00 | O |
| ATOM | 6122 | CB | CYS | C | 285 | 50.293 | 46.588 | −12.868 | 1.00 | 39.48 | C |
| ATOM | 6123 | SG | CYS | C | 285 | 49.730 | 48.177 | −12.207 | 1.00 | 49.36 | S |
| ATOM | 6124 | N | TRP | C | 286 | 51.710 | 44.576 | −15.061 | 1.00 | 37.34 | N |
| ATOM | 6125 | CA | TRP | C | 286 | 52.168 | 43.199 | −15.261 | 1.00 | 32.71 | C |
| ATOM | 6126 | C | TRP | C | 286 | 53.205 | 43.017 | −16.356 | 1.00 | 32.80 | C |
| ATOM | 6127 | O | TRP | C | 286 | 53.891 | 42.006 | −16.400 | 1.00 | 39.79 | O |
| ATOM | 6128 | CB | TRP | C | 286 | 50.994 | 42.278 | −15.555 | 1.00 | 28.57 | C |
| ATOM | 6129 | CG | TRP | C | 286 | 50.342 | 41.769 | −14.346 | 1.00 | 25.42 | C |
| ATOM | 6130 | CD1 | TRP | C | 286 | 49.094 | 42.053 | −13.923 | 1.00 | 27.38 | C |
| ATOM | 6131 | CD2 | TRP | C | 286 | 50.906 | 40.880 | −13.378 | 1.00 | 26.93 | C |
| ATOM | 6132 | NE1 | TRP | C | 286 | 48.830 | 41.392 | −12.756 | 1.00 | 28.26 | N |
| ATOM | 6133 | CE2 | TRP | C | 286 | 49.929 | 40.661 | −12.398 | 1.00 | 24.74 | C |
| ATOM | 6134 | CE3 | TRP | C | 286 | 52.149 | 40.251 | −13.244 | 1.00 | 32.49 | C |
| ATOM | 6135 | CZ2 | TRP | C | 286 | 50.147 | 39.842 | −11.285 | 1.00 | 27.37 | C |
| ATOM | 6136 | CZ3 | TRP | C | 286 | 52.371 | 39.436 | −12.139 | 1.00 | 28.94 | C |
| ATOM | 6137 | CH2 | TRP | C | 286 | 51.372 | 39.241 | −11.173 | 1.00 | 28.95 | C |
| ATOM | 6138 | N | LEU | C | 287 | 53.318 | 43.978 | −17.252 | 1.00 | 30.59 | N |
| ATOM | 6139 | CA | LEU | C | 287 | 54.184 | 43.771 | −18.396 | 1.00 | 37.19 | C |
| ATOM | 6140 | C | LEU | C | 287 | 55.659 | 43.711 | −18.000 | 1.00 | 43.38 | C |
| ATOM | 6141 | O | LEU | C | 287 | 56.378 | 42.823 | −18.459 | 1.00 | 48.76 | O |
| ATOM | 6142 | CB | LEU | C | 287 | 53.930 | 44.808 | −19.498 | 1.00 | 33.25 | C |
| ATOM | 6143 | CG | LEU | C | 287 | 54.582 | 44.476 | −20.842 | 1.00 | 50.14 | C |
| ATOM | 6144 | CD1 | LEU | C | 287 | 54.276 | 43.037 | −21.293 | 1.00 | 42.55 | C |
| ATOM | 6145 | CD2 | LEU | C | 287 | 54.150 | 45.484 | −21.897 | 1.00 | 44.25 | C |
| ATOM | 6146 | N | PRO | C | 288 | 56.120 | 44.644 | −17.144 | 1.00 | 42.07 | N |
| ATOM | 6147 | CA | PRO | C | 288 | 57.533 | 44.566 | −16.752 | 1.00 | 44.53 | C |
| ATOM | 6148 | C | PRO | C | 288 | 57.922 | 43.153 | −16.335 | 1.00 | 41.31 | C |
| ATOM | 6149 | O | PRO | C | 288 | 58.942 | 42.628 | −16.799 | 1.00 | 40.08 | O |
| ATOM | 6150 | CB | PRO | C | 288 | 57.620 | 45.525 | −15.563 | 1.00 | 33.71 | C |
| ATOM | 6151 | CG | PRO | C | 288 | 56.574 | 46.555 | −15.856 | 1.00 | 38.66 | C |
| ATOM | 6152 | CD | PRO | C | 288 | 55.445 | 45.810 | −16.542 | 1.00 | 40.76 | C |
| ATOM | 6153 | N | PHE | C | 289 | 57.099 | 42.541 | −15.490 | 1.00 | 36.77 | N |
| ATOM | 6154 | CA | PHE | C | 289 | 57.363 | 41.189 | −15.011 | 1.00 | 36.16 | C |
| ATOM | 6155 | C | PHE | C | 289 | 57.486 | 40.161 | −16.141 | 1.00 | 38.33 | C |
| ATOM | 6156 | O | PHE | C | 289 | 58.434 | 39.377 | −16.167 | 1.00 | 41.88 | O |
| ATOM | 6157 | CB | PHE | C | 289 | 56.295 | 40.762 | −14.005 | 1.00 | 31.19 | C |
| ATOM | 6158 | CG | PHE | C | 289 | 56.410 | 39.332 | −13.566 | 1.00 | 30.45 | C |
| ATOM | 6159 | CD1 | PHE | C | 289 | 57.195 | 38.985 | −12.474 | 1.00 | 29.48 | C |
| ATOM | 6160 | CD2 | PHE | C | 289 | 55.724 | 38.329 | −14.240 | 1.00 | 31.94 | C |
| ATOM | 6161 | CE1 | PHE | C | 289 | 57.302 | 37.665 | −12.059 | 1.00 | 23.99 | C |
| ATOM | 6162 | CE2 | PHE | C | 289 | 55.823 | 37.008 | −13.833 | 1.00 | 27.89 | C |
| ATOM | 6163 | CZ | PHE | C | 289 | 56.616 | 36.678 | −12.740 | 1.00 | 27.06 | C |
| ATOM | 6164 | N | PHE | C | 290 | 56.541 | 40.163 | −17.076 | 1.00 | 35.54 | N |
| ATOM | 6165 | CA | PHE | C | 290 | 56.595 | 39.199 | −18.176 | 1.00 | 38.64 | C |
| ATOM | 6166 | C | PHE | C | 290 | 57.701 | 39.479 | −19.175 | 1.00 | 38.15 | C |
| ATOM | 6167 | O | PHE | C | 290 | 58.231 | 38.545 | −19.783 | 1.00 | 38.83 | O |
| ATOM | 6168 | CB | PHE | C | 290 | 55.237 | 39.041 | −18.860 | 1.00 | 33.36 | C |
| ATOM | 6169 | CG | PHE | C | 290 | 54.264 | 38.276 | −18.038 | 1.00 | 31.42 | C |
| ATOM | 6170 | CD1 | PHE | C | 290 | 53.268 | 38.936 | −17.332 | 1.00 | 31.11 | C |
| ATOM | 6171 | CD2 | PHE | C | 290 | 54.381 | 36.899 | −17.915 | 1.00 | 29.34 | C |
| ATOM | 6172 | CE1 | PHE | C | 290 | 52.382 | 38.228 | −16.546 | 1.00 | 30.33 | C |
| ATOM | 6173 | CE2 | PHE | C | 290 | 53.505 | 36.178 | −17.121 | 1.00 | 25.35 | C |
| ATOM | 6174 | CZ | PHE | C | 290 | 52.502 | 36.837 | −16.442 | 1.00 | 29.59 | C |
| ATOM | 6175 | N | LEU | C | 291 | 58.054 | 40.752 | −19.341 | 1.00 | 34.51 | N |
| ATOM | 6176 | CA | LEU | C | 291 | 59.242 | 41.095 | −20.117 | 1.00 | 37.90 | C |
| ATOM | 6177 | C | LEU | C | 291 | 60.463 | 40.468 | −19.455 | 1.00 | 47.61 | C |
| ATOM | 6178 | O | LEU | C | 291 | 61.218 | 39.728 | −20.097 | 1.00 | 38.90 | O |
| ATOM | 6179 | CB | LEU | C | 291 | 59.434 | 42.605 | −20.214 | 1.00 | 40.65 | C |
| ATOM | 6180 | CG | LEU | C | 291 | 58.500 | 43.364 | −21.152 | 1.00 | 58.43 | C |
| ATOM | 6181 | CD1 | LEU | C | 291 | 59.184 | 44.642 | −21.627 | 1.00 | 48.77 | C |
| ATOM | 6182 | CD2 | LEU | C | 291 | 58.111 | 42.490 | −22.336 | 1.00 | 36.08 | C |
| ATOM | 6183 | N | VAL | C | 292 | 60.641 | 40.756 | −18.163 | 1.00 | 41.33 | N |
| ATOM | 6184 | CA | VAL | C | 292 | 61.796 | 40.262 | −17.422 | 1.00 | 46.37 | C |
| ATOM | 6185 | C | VAL | C | 292 | 61.837 | 38.739 | −17.431 | 1.00 | 42.06 | C |
| ATOM | 6186 | O | VAL | C | 292 | 62.900 | 38.130 | −17.421 | 1.00 | 42.24 | O |
| ATOM | 6187 | CB | VAL | C | 292 | 61.804 | 40.794 | −15.975 | 1.00 | 48.73 | C |
| ATOM | 6188 | CG1 | VAL | C | 292 | 62.638 | 39.897 | −15.077 | 1.00 | 58.93 | C |
| ATOM | 6189 | CG2 | VAL | C | 292 | 62.334 | 42.213 | −15.945 | 1.00 | 49.23 | C |
| ATOM | 6190 | N | ASN | C | 293 | 60.661 | 38.137 | −17.468 | 1.00 | 45.58 | N |
| ATOM | 6191 | CA | ASN | C | 293 | 60.545 | 36.692 | −17.489 | 1.00 | 51.02 | C |
| ATOM | 6192 | C | ASN | C | 293 | 61.188 | 36.079 | −18.728 | 1.00 | 49.09 | C |
| ATOM | 6193 | O | ASN | C | 293 | 61.848 | 35.040 | −18.656 | 1.00 | 49.94 | O |
| ATOM | 6194 | CB | ASN | C | 293 | 59.073 | 36.293 | −17.421 | 1.00 | 49.15 | C |
| ATOM | 6195 | CG | ASN | C | 293 | 58.870 | 34.957 | −16.752 | 1.00 | 56.39 | C |
| ATOM | 6196 | OD1 | ASN | C | 293 | 59.432 | 34.689 | −15.684 | 1.00 | 53.12 | O |
| ATOM | 6197 | ND2 | ASN | C | 293 | 58.067 | 34.102 | −17.378 | 1.00 | 53.08 | N |
| ATOM | 6198 | N | ILE | C | 294 | 60.993 | 36.722 | −19.872 | 1.00 | 50.65 | N |
| ATOM | 6199 | CA | ILE | C | 294 | 61.531 | 36.191 | −21.119 | 1.00 | 48.96 | C |
| ATOM | 6200 | C | ILE | C | 294 | 63.022 | 36.472 | −21.274 | 1.00 | 40.86 | C |
| ATOM | 6201 | O | ILE | C | 294 | 63.795 | 35.563 | −21.581 | 1.00 | 36.11 | O |
| ATOM | 6202 | CB | ILE | C | 294 | 60.771 | 36.725 | −22.323 | 1.00 | 47.06 | C |
| ATOM | 6203 | CG1 | ILE | C | 294 | 59.295 | 36.343 | −22.214 | 1.00 | 42.91 | C |
| ATOM | 6204 | CG2 | ILE | C | 294 | 61.368 | 36.164 | −23.602 | 1.00 | 56.91 | C |
| ATOM | 6205 | CD1 | ILE | C | 294 | 58.409 | 37.161 | −23.117 | 1.00 | 48.12 | C |
| ATOM | 6206 | N | VAL | C | 295 | 63.416 | 37.726 | −21.055 | 1.00 | 40.67 | N |
| ATOM | 6207 | CA | VAL | C | 295 | 64.826 | 38.099 | −21.036 | 1.00 | 35.22 | C |
| ATOM | 6208 | C | VAL | C | 295 | 65.647 | 37.060 | −20.289 | 1.00 | 39.35 | C |
| ATOM | 6209 | O | VAL | C | 295 | 66.676 | 36.617 | −20.774 | 1.00 | 45.85 | O |
| ATOM | 6210 | CB | VAL | C | 295 | 65.059 | 39.467 | −20.376 | 1.00 | 32.86 | C |
| ATOM | 6211 | CG1 | VAL | C | 295 | 66.518 | 39.619 | −20.003 | 1.00 | 38.15 | C |
| ATOM | 6212 | CG2 | VAL | C | 295 | 64.641 | 40.584 | −21.304 | 1.00 | 27.14 | C |
| ATOM | 6213 | N | ASN | C | 296 | 65.176 | 36.672 | −19.110 | 1.00 | 39.32 | N |
| ATOM | 6214 | CA | ASN | C | 296 | 65.838 | 35.655 | −18.304 | 1.00 | 46.96 | C |
| ATOM | 6215 | C | ASN | C | 296 | 66.082 | 34.332 | −19.027 | 1.00 | 49.64 | C |
| ATOM | 6216 | O | ASN | C | 296 | 67.129 | 33.702 | −18.854 | 1.00 | 55.79 | O |
| ATOM | 6217 | CB | ASN | C | 296 | 65.053 | 35.396 | −17.015 | 1.00 | 49.24 | C |
| ATOM | 6218 | CG | ASN | C | 296 | 65.811 | 35.818 | −15.776 | 1.00 | 64.80 | C |
| ATOM | 6219 | OD1 | ASN | C | 296 | 66.966 | 36.257 | −15.848 | 1.00 | 65.41 | O |
| ATOM | 6220 | ND2 | ASN | C | 296 | 65.167 | 35.683 | −14.624 | 1.00 | 83.24 | N |
| ATOM | 6221 | N | VAL | C | 297 | 65.108 | 33.899 | −19.815 | 1.00 | 46.00 | N |
| ATOM | 6222 | CA | VAL | C | 297 | 65.236 | 32.645 | −20.537 | 1.00 | 55.34 | C |
| ATOM | 6223 | C | VAL | C | 297 | 66.475 | 32.674 | −21.435 | 1.00 | 61.06 | C |
| ATOM | 6224 | O | VAL | C | 297 | 67.177 | 31.673 | −21.583 | 1.00 | 49.34 | O |
| ATOM | 6225 | CB | VAL | C | 297 | 63.981 | 32.356 | −21.375 | 1.00 | 46.45 | C |
| ATOM | 6226 | CG1 | VAL | C | 297 | 64.148 | 31.065 | −22.159 | 1.00 | 44.51 | C |
| ATOM | 6227 | CG2 | VAL | C | 297 | 62.762 | 32.277 | −20.475 | 1.00 | 53.06 | C |
| ATOM | 6228 | N | PHE | C | 298 | 66.746 | 33.841 | −22.010 | 1.00 | 56.58 | N |
| ATOM | 6229 | CA | PHE | C | 298 | 67.868 | 34.022 | −22.919 | 1.00 | 60.95 | C |
| ATOM | 6230 | C | PHE | C | 298 | 69.183 | 34.147 | −22.164 | 1.00 | 65.01 | C |
| ATOM | 6231 | O | PHE | C | 298 | 70.184 | 33.526 | −22.523 | 1.00 | 79.89 | O |
| ATOM | 6232 | CB | PHE | C | 298 | 67.643 | 35.269 | −23.771 | 1.00 | 59.40 | C |
| ATOM | 6233 | CG | PHE | C | 298 | 66.521 | 35.132 | −24.759 | 1.00 | 61.55 | C |
| ATOM | 6234 | CD1 | PHE | C | 298 | 65.654 | 34.053 | −24.701 | 1.00 | 74.26 | C |
| ATOM | 6235 | CD2 | PHE | C | 298 | 66.318 | 36.096 | −25.731 | 1.00 | 85.59 | C |
| ATOM | 6236 | CE1 | PHE | C | 298 | 64.616 | 33.923 | −25.607 | 1.00 | 70.58 | C |
| ATOM | 6237 | CE2 | PHE | C | 298 | 65.279 | 35.976 | −26.639 | 1.00 | 95.91 | C |
| ATOM | 6238 | CZ | PHE | C | 298 | 64.428 | 34.885 | −26.576 | 1.00 | 82.51 | C |
| ATOM | 6239 | N | ASN | C | 299 | 69.169 | 34.944 | −21.105 | 1.00 | 62.91 | N |
| ATOM | 6240 | CA | ASN | C | 299 | 70.381 | 35.272 | −20.378 | 1.00 | 61.38 | C |
| ATOM | 6241 | C | ASN | C | 299 | 70.134 | 35.434 | −18.887 | 1.00 | 73.98 | C |
| ATOM | 6242 | O | ASN | C | 299 | 69.970 | 36.560 | −18.415 | 1.00 | 73.08 | O |
| ATOM | 6243 | CB | ASN | C | 299 | 70.951 | 36.575 | −20.928 | 1.00 | 71.52 | C |
| ATOM | 6244 | CG | ASN | C | 299 | 72.221 | 36.992 | −20.236 | 1.00 | 70.07 | C |
| ATOM | 6245 | OD1 | ASN | C | 299 | 72.895 | 36.180 | −19.606 | 1.00 | 78.04 | O |
| ATOM | 6246 | ND2 | ASN | C | 299 | 72.566 | 38.263 | −20.362 | 1.00 | 74.40 | N |
| ATOM | 6247 | N | ARG | C | 300 | 70.095 | 34.318 | −18.152 | 1.00 | 90.87 | N |
| ATOM | 6248 | CA | ARG | C | 300 | 70.008 | 34.367 | −16.690 | 1.00 | 70.01 | C |
| ATOM | 6249 | C | ARG | C | 300 | 71.155 | 35.238 | −16.216 | 1.00 | 73.77 | C |
| ATOM | 6250 | O | ARG | C | 300 | 72.230 | 35.202 | −16.802 | 1.00 | 93.16 | O |
| ATOM | 6251 | CB | ARG | C | 300 | 70.121 | 32.966 | −16.066 | 1.00 | 66.50 | C |
| ATOM | 6252 | CG | ARG | C | 300 | 69.268 | 31.877 | −16.744 | 1.00 | 92.81 | C |
| ATOM | 6253 | CD | ARG | C | 300 | 68.842 | 30.762 | −15.768 | 1.00 | 105.79 | C |
| ATOM | 6254 | NE | ARG | C | 300 | 68.038 | 29.716 | −16.413 | 1.00 | 113.60 | N |
| ATOM | 6255 | CZ | ARG | C | 300 | 67.270 | 28.836 | −15.766 | 1.00 | 110.73 | C |
| ATOM | 6256 | NH1 | ARG | C | 300 | 67.179 | 28.861 | −14.442 | 1.00 | 86.83 | N |
| ATOM | 6257 | NH2 | ARG | C | 300 | 66.583 | 27.925 | −16.447 | 1.00 | 102.03 | N |
| ATOM | 6258 | N | ASP | C | 301 | 70.926 | 36.039 | −15.182 | 1.00 | 79.53 | N |
| ATOM | 6259 | CA | ASP | C | 301 | 71.980 | 36.881 | −14.599 | 1.00 | 88.71 | C |
| ATOM | 6260 | C | ASP | C | 301 | 72.269 | 38.173 | −15.371 | 1.00 | 81.94 | C |
| ATOM | 6261 | O | ASP | C | 301 | 73.365 | 38.719 | −15.275 | 1.00 | 81.89 | O |
| ATOM | 6262 | CB | ASP | C | 301 | 73.293 | 36.104 | −14.434 | 1.00 | 83.67 | C |
| ATOM | 6263 | CG | ASP | C | 301 | 73.095 | 34.732 | −13.812 | 1.00 | 106.75 | C |
| ATOM | 6264 | OD1 | ASP | C | 301 | 72.131 | 34.555 | −13.031 | 1.00 | 109.74 | O |
| ATOM | 6265 | OD2 | ASP | C | 301 | 73.917 | 33.833 | −14.108 | 1.00 | 99.09 | O |
| ATOM | 6266 | N | LEU | C | 302 | 71.301 | 38.658 | −16.137 | 1.00 | 74.01 | N |
| ATOM | 6267 | CA | LEU | C | 302 | 71.423 | 39.979 | −16.738 | 1.00 | 72.68 | C |
| ATOM | 6268 | C | LEU | C | 302 | 70.570 | 40.993 | −15.982 | 1.00 | 89.27 | C |
| ATOM | 6269 | O | LEU | C | 302 | 70.799 | 42.204 | −16.066 | 1.00 | 90.09 | O |
| ATOM | 6270 | CB | LEU | C | 302 | 70.986 | 39.969 | −18.193 | 1.00 | 75.58 | C |
| ATOM | 6271 | CG | LEU | C | 302 | 70.948 | 41.406 | −18.708 | 1.00 | 70.89 | C |
| ATOM | 6272 | CD1 | LEU | C | 302 | 72.366 | 41.871 | −19.010 | 1.00 | 80.44 | C |
| ATOM | 6273 | CD2 | LEU | C | 302 | 70.050 | 41.548 | −19.921 | 1.00 | 74.40 | C |
| ATOM | 6274 | N | VAL | C | 303 | 69.571 | 40.491 | −15.262 | 1.00 | 85.01 | N |
| ATOM | 6275 | CA | VAL | C | 303 | 68.719 | 41.334 | −14.427 | 1.00 | 66.46 | C |
| ATOM | 6276 | C | VAL | C | 303 | 68.571 | 40.718 | −13.030 | 1.00 | 72.63 | C |
| ATOM | 6277 | O | VAL | C | 303 | 68.351 | 39.508 | −12.894 | 1.00 | 71.79 | O |
| ATOM | 6278 | CB | VAL | C | 303 | 67.352 | 41.598 | −15.099 | 1.00 | 74.75 | C |
| ATOM | 6279 | CG1 | VAL | C | 303 | 66.311 | 42.030 | −14.078 | 1.00 | 66.62 | C |
| ATOM | 6280 | CG2 | VAL | C | 303 | 67.506 | 42.649 | −16.190 | 1.00 | 77.61 | C |
| ATOM | 6281 | N | PRO | C | 304 | 68.718 | 41.557 | −11.988 | 1.00 | 65.84 | N |
| ATOM | 6282 | CA | PRO | C | 304 | 68.854 | 41.175 | −10.576 | 1.00 | 49.65 | C |
| ATOM | 6283 | C | PRO | C | 304 | 67.594 | 40.600 | −9.919 | 1.00 | 54.23 | C |
| ATOM | 6284 | O | PRO | C | 304 | 66.540 | 41.241 | −9.932 | 1.00 | 47.94 | O |
| ATOM | 6285 | CB | PRO | C | 304 | 69.226 | 42.502 | −9.911 | 1.00 | 41.78 | C |
| ATOM | 6286 | CG | PRO | C | 304 | 68.629 | 43.521 | −10.800 | 1.00 | 45.56 | C |
| ATOM | 6287 | CD | PRO | C | 304 | 68.928 | 43.004 | −12.160 | 1.00 | 56.24 | C |
| ATOM | 6288 | N | ASP | C | 305 | 67.736 | 39.408 | −9.335 | 1.00 | 55.16 | N |
| ATOM | 6289 | CA | ASP | C | 305 | 66.670 | 38.718 | −8.597 | 1.00 | 63.50 | C |
| ATOM | 6290 | C | ASP | C | 305 | 65.721 | 39.633 | −7.816 | 1.00 | 58.41 | C |
| ATOM | 6291 | O | ASP | C | 305 | 64.517 | 39.376 | −7.741 | 1.00 | 49.56 | O |
| ATOM | 6292 | CB | ASP | C | 305 | 67.275 | 37.708 | −7.611 | 1.00 | 74.46 | C |
| ATOM | 6293 | CG | ASP | C | 305 | 67.573 | 36.358 | −8.246 | 1.00 | 97.46 | C |
| ATOM | 6294 | OD1 | ASP | C | 305 | 66.833 | 35.935 | −9.161 | 1.00 | 97.26 | O |
| ATOM | 6295 | OD2 | ASP | C | 305 | 68.547 | 35.707 | −7.808 | 1.00 | 111.82 | O |
| ATOM | 6296 | N | TRP | C | 306 | 66.269 | 40.676 | −7.203 | 1.00 | 51.66 | N |
| ATOM | 6297 | CA | TRP | C | 306 | 65.469 | 41.541 | −6.354 | 1.00 | 49.78 | C |
| ATOM | 6298 | C | TRP | C | 306 | 64.504 | 42.402 | −7.168 | 1.00 | 46.94 | C |
| ATOM | 6299 | O | TRP | C | 306 | 63.426 | 42.750 | −6.690 | 1.00 | 47.32 | O |
| ATOM | 6300 | CB | TRP | C | 306 | 66.370 | 42.417 | −5.483 | 1.00 | 52.16 | C |
| ATOM | 6301 | CG | TRP | C | 306 | 67.153 | 43.406 | −6.263 | 1.00 | 50.19 | C |
| ATOM | 6302 | CD1 | TRP | C | 306 | 68.449 | 43.287 | −6.667 | 1.00 | 54.38 | C |
| ATOM | 6303 | CD2 | TRP | C | 306 | 66.691 | 44.670 | −6.752 | 1.00 | 44.18 | C |
| ATOM | 6304 | NE1 | TRP | C | 306 | 68.825 | 44.403 | −7.376 | 1.00 | 59.74 | N |
| ATOM | 6305 | CE2 | TRP | C | 306 | 67.763 | 45.266 | −7.445 | 1.00 | 51.21 | C |
| ATOM | 6306 | CE3 | TRP | C | 306 | 65.474 | 45.352 | −6.676 | 1.00 | 44.60 | C |
| ATOM | 6307 | CZ2 | TRP | C | 306 | 67.659 | 46.515 | −8.053 | 1.00 | 45.41 | C |
| ATOM | 6308 | CZ3 | TRP | C | 306 | 65.369 | 46.592 | −7.278 | 1.00 | 54.74 | C |
| ATOM | 6309 | CH2 | TRP | C | 306 | 66.457 | 47.162 | −7.959 | 1.00 | 55.43 | C |
| ATOM | 6310 | N | LEU | C | 307 | 64.901 | 42.744 | −8.392 | 1.00 | 42.95 | N |
| ATOM | 6311 | CA | LEU | C | 307 | 64.054 | 43.508 | −9.302 | 1.00 | 42.76 | C |
| ATOM | 6312 | C | LEU | C | 307 | 63.013 | 42.582 | −9.918 | 1.00 | 39.67 | C |
| ATOM | 6313 | O | LEU | C | 307 | 61.913 | 42.989 | −10.272 | 1.00 | 34.27 | O |
| ATOM | 6314 | CB | LEU | C | 307 | 64.900 | 44.135 | −10.402 | 1.00 | 48.73 | C |
| ATOM | 6315 | CG | LEU | C | 307 | 64.128 | 44.887 | −11.484 | 1.00 | 50.45 | C |
| ATOM | 6316 | CD1 | LEU | C | 307 | 63.328 | 46.038 | −10.887 | 1.00 | 44.45 | C |
| ATOM | 6317 | CD2 | LEU | C | 307 | 65.079 | 45.386 | −12.559 | 1.00 | 54.50 | C |
| ATOM | 6318 | N | PHE | C | 308 | 63.385 | 41.320 | −10.046 | 1.00 | 44.98 | N |
| ATOM | 6319 | CA | PHE | C | 308 | 62.439 | 40.292 | −10.408 | 1.00 | 42.10 | C |
| ATOM | 6320 | C | PHE | C | 308 | 61.298 | 40.358 | −9.393 | 1.00 | 35.26 | C |
| ATOM | 6321 | O | PHE | C | 308 | 60.137 | 40.510 | −9.750 | 1.00 | 37.60 | O |
| ATOM | 6322 | CB | PHE | C | 308 | 63.135 | 38.928 | −10.367 | 1.00 | 53.39 | C |
| ATOM | 6323 | CG | PHE | C | 308 | 62.489 | 37.890 | −11.229 | 1.00 | 61.90 | C |
| ATOM | 6324 | CD1 | PHE | C | 308 | 62.831 | 37.775 | −12.564 | 1.00 | 59.56 | C |
| ATOM | 6325 | CD2 | PHE | C | 308 | 61.539 | 37.022 | −10.701 | 1.00 | 76.20 | C |
| ATOM | 6326 | CE1 | PHE | C | 308 | 62.231 | 36.822 | −13.367 | 1.00 | 67.42 | C |
| ATOM | 6327 | CE2 | PHE | C | 308 | 60.936 | 36.061 | −11.499 | 1.00 | 76.53 | C |
| ATOM | 6328 | CZ | PHE | C | 308 | 61.284 | 35.964 | −12.836 | 1.00 | 66.84 | C |
| ATOM | 6329 | N | VAL | C | 309 | 61.643 | 40.276 | −8.117 | 1.00 | 44.16 | N |
| ATOM | 6330 | CA | VAL | C | 309 | 60.649 | 40.339 | −7.047 | 1.00 | 48.42 | C |
| ATOM | 6331 | C | VAL | C | 309 | 59.876 | 41.665 | −7.017 | 1.00 | 41.97 | C |
| ATOM | 6332 | O | VAL | C | 309 | 58.642 | 41.672 | −6.901 | 1.00 | 36.14 | O |
| ATOM | 6333 | CB | VAL | C | 309 | 61.287 | 40.042 | −5.673 | 1.00 | 45.87 | C |
| ATOM | 6334 | CG1 | VAL | C | 309 | 60.322 | 40.362 | −4.549 | 1.00 | 49.31 | C |
| ATOM | 6335 | CG2 | VAL | C | 309 | 61.706 | 38.586 | −5.601 | 1.00 | 36.42 | C |
| ATOM | 6336 | N | ALA | C | 310 | 60.601 | 42.777 | −7.127 | 1.00 | 41.28 | N |
| ATOM | 6337 | CA | ALA | C | 310 | 59.987 | 44.107 | −7.217 | 1.00 | 40.66 | C |
| ATOM | 6338 | C | ALA | C | 310 | 58.847 | 44.135 | −8.226 | 1.00 | 37.77 | C |
| ATOM | 6339 | O | ALA | C | 310 | 57.735 | 44.556 | −7.908 | 1.00 | 26.79 | O |
| ATOM | 6340 | CB | ALA | C | 310 | 61.024 | 45.145 | −7.590 | 1.00 | 33.14 | C |
| ATOM | 6341 | N | PHE | C | 311 | 59.138 | 43.682 | −9.443 | 1.00 | 34.24 | N |
| ATOM | 6342 | CA | PHE | C | 311 | 58.153 | 43.681 | −10.523 | 1.00 | 33.84 | C |
| ATOM | 6343 | C | PHE | C | 311 | 56.940 | 42.767 | −10.273 | 1.00 | 30.30 | C |
| ATOM | 6344 | O | PHE | C | 311 | 55.817 | 43.099 | −10.642 | 1.00 | 26.32 | O |
| ATOM | 6345 | CB | PHE | C | 311 | 58.823 | 43.342 | −11.862 | 1.00 | 31.50 | C |
| ATOM | 6346 | CG | PHE | C | 311 | 59.509 | 44.515 | −12.514 | 1.00 | 36.43 | C |
| ATOM | 6347 | CD1 | PHE | C | 311 | 60.598 | 44.320 | −13.355 | 1.00 | 35.51 | C |
| ATOM | 6348 | CD2 | PHE | C | 311 | 59.071 | 45.815 | −12.275 | 1.00 | 32.14 | C |
| ATOM | 6349 | CE1 | PHE | C | 311 | 61.230 | 45.401 | −13.953 | 1.00 | 45.19 | C |
| ATOM | 6350 | CE2 | PHE | C | 311 | 59.696 | 46.900 | −12.869 | 1.00 | 34.05 | C |
| ATOM | 6351 | CZ | PHE | C | 311 | 60.776 | 46.698 | −13.709 | 1.00 | 44.86 | C |
| ATOM | 6352 | N | ASN | C | 312 | 57.165 | 41.620 | −9.648 | 1.00 | 28.20 | N |
| ATOM | 6353 | CA | ASN | C | 312 | 56.065 | 40.725 | −9.342 | 1.00 | 27.13 | C |
| ATOM | 6354 | C | ASN | C | 312 | 55.109 | 41.352 | −8.306 | 1.00 | 32.75 | C |
| ATOM | 6355 | O | ASN | C | 312 | 53.899 | 41.110 | −8.334 | 1.00 | 30.23 | O |
| ATOM | 6356 | CB | ASN | C | 312 | 56.612 | 39.372 | −8.871 | 1.00 | 23.33 | C |
| ATOM | 6357 | CG | ASN | C | 312 | 55.554 | 38.274 | −8.849 | 1.00 | 28.58 | C |
| ATOM | 6358 | OD1 | ASN | C | 312 | 55.873 | 37.104 | −8.641 | 1.00 | 27.91 | O |
| ATOM | 6359 | ND2 | ASN | C | 312 | 54.295 | 38.644 | −9.052 | 1.00 | 35.12 | N |
| ATOM | 6360 | N | TRP | C | 313 | 55.649 | 42.167 | −7.401 | 1.00 | 31.63 | N |
| ATOM | 6361 | CA | TRP | C | 313 | 54.820 | 42.885 | −6.427 | 1.00 | 29.87 | C |
| ATOM | 6362 | C | TRP | C | 313 | 54.049 | 44.051 | −7.046 | 1.00 | 27.76 | C |
| ATOM | 6363 | O | TRP | C | 313 | 53.008 | 44.470 | −6.532 | 1.00 | 27.77 | O |
| ATOM | 6364 | CB | TRP | C | 313 | 55.647 | 43.334 | −5.209 | 1.00 | 27.90 | C |
| ATOM | 6365 | CG | TRP | C | 313 | 55.798 | 42.231 | −4.224 | 1.00 | 30.93 | C |
| ATOM | 6366 | CD1 | TRP | C | 313 | 56.776 | 41.281 | −4.199 | 1.00 | 34.09 | C |
| ATOM | 6367 | CD2 | TRP | C | 313 | 54.904 | 41.914 | −3.155 | 1.00 | 29.65 | C |
| ATOM | 6368 | NE1 | TRP | C | 313 | 56.557 | 40.404 | −3.164 | 1.00 | 30.84 | N |
| ATOM | 6369 | CE2 | TRP | C | 313 | 55.410 | 40.771 | −2.512 | 1.00 | 33.11 | C |
| ATOM | 6370 | CE3 | TRP | C | 313 | 53.728 | 42.488 | −2.677 | 1.00 | 27.25 | C |
| ATOM | 6371 | CZ2 | TRP | C | 313 | 54.782 | 40.200 | −1.414 | 1.00 | 34.75 | C |
| ATOM | 6372 | CZ3 | TRP | C | 313 | 53.110 | 41.919 | −1.587 | 1.00 | 29.79 | C |
| ATOM | 6373 | CH2 | TRP | C | 313 | 53.636 | 40.792 | −0.966 | 1.00 | 32.30 | C |
| ATOM | 6374 | N | LEU | C | 314 | 54.569 | 44.568 | −8.153 | 1.00 | 25.04 | N |
| ATOM | 6375 | CA | LEU | C | 314 | 53.854 | 45.559 | −8.929 | 1.00 | 26.40 | C |
| ATOM | 6376 | C | LEU | C | 314 | 52.647 | 44.889 | −9.586 | 1.00 | 31.77 | C |
| ATOM | 6377 | O | LEU | C | 314 | 51.585 | 45.486 | −9.717 | 1.00 | 31.16 | O |
| ATOM | 6378 | CB | LEU | C | 314 | 54.771 | 46.162 | −9.987 | 1.00 | 24.36 | C |
| ATOM | 6379 | CG | LEU | C | 314 | 54.045 | 47.206 | −10.830 | 1.00 | 27.12 | C |
| ATOM | 6380 | CD1 | LEU | C | 314 | 53.487 | 48.304 | −9.939 | 1.00 | 29.73 | C |
| ATOM | 6381 | CD2 | LEU | C | 314 | 54.971 | 47.784 | −11.865 | 1.00 | 31.44 | C |
| ATOM | 6382 | N | GLY | C | 315 | 52.815 | 43.635 | −9.991 | 1.00 | 31.34 | N |
| ATOM | 6383 | CA | GLY | C | 315 | 51.706 | 42.864 | −10.507 | 1.00 | 25.48 | C |
| ATOM | 6384 | C | GLY | C | 315 | 50.670 | 42.599 | −9.433 | 1.00 | 26.89 | C |
| ATOM | 6385 | O | GLY | C | 315 | 49.471 | 42.680 | −9.678 | 1.00 | 29.28 | O |
| ATOM | 6386 | N | TYR | C | 316 | 51.136 | 42.267 | −8.234 | 1.00 | 29.58 | N |
| ATOM | 6387 | CA | TYR | C | 316 | 50.237 | 41.995 | −7.119 | 1.00 | 34.76 | C |
| ATOM | 6388 | C | TYR | C | 316 | 49.427 | 43.225 | −6.732 | 1.00 | 37.34 | C |
| ATOM | 6389 | O | TYR | C | 316 | 48.247 | 43.117 | −6.403 | 1.00 | 39.83 | O |
| ATOM | 6390 | CB | TYR | C | 316 | 51.012 | 41.529 | −5.895 | 1.00 | 36.25 | C |
| ATOM | 6391 | CG | TYR | C | 316 | 51.539 | 40.118 | −5.958 | 1.00 | 35.61 | C |
| ATOM | 6392 | CD1 | TYR | C | 316 | 52.636 | 39.745 | −5.191 | 1.00 | 32.49 | C |
| ATOM | 6393 | CD2 | TYR | C | 316 | 50.943 | 39.157 | −6.768 | 1.00 | 31.64 | C |
| ATOM | 6394 | CE1 | TYR | C | 316 | 53.128 | 38.464 | −5.225 | 1.00 | 37.76 | C |
| ATOM | 6395 | CE2 | TYR | C | 316 | 51.435 | 37.860 | −6.814 | 1.00 | 35.73 | C |
| ATOM | 6396 | CZ | TYR | C | 316 | 52.534 | 37.525 | −6.038 | 1.00 | 38.35 | C |
| ATOM | 6397 | OH | TYR | C | 316 | 53.056 | 36.258 | −6.048 | 1.00 | 33.92 | O |
| ATOM | 6398 | N | ALA | C | 317 | 50.072 | 44.388 | −6.752 | 1.00 | 41.70 | N |
| ATOM | 6399 | CA | ALA | C | 317 | 49.429 | 45.640 | −6.350 | 1.00 | 44.41 | C |
| ATOM | 6400 | C | ALA | C | 317 | 48.274 | 46.025 | −7.276 | 1.00 | 36.59 | C |
| ATOM | 6401 | O | ALA | C | 317 | 47.293 | 46.624 | −6.843 | 1.00 | 39.89 | O |
| ATOM | 6402 | CB | ALA | C | 317 | 50.460 | 46.772 | −6.258 | 1.00 | 30.43 | C |
| ATOM | 6403 | N | ASN | C | 318 | 48.394 | 45.682 | −8.549 | 1.00 | 27.70 | N |
| ATOM | 6404 | CA | ASN | C | 318 | 47.294 | 45.875 | −9.471 | 1.00 | 37.90 | C |
| ATOM | 6405 | C | ASN | C | 318 | 45.962 | 45.489 | −8.817 | 1.00 | 36.19 | C |
| ATOM | 6406 | O | ASN | C | 318 | 44.959 | 46.167 | −8.998 | 1.00 | 43.02 | O |
| ATOM | 6407 | CB | ASN | C | 318 | 47.524 | 45.047 | −10.737 | 1.00 | 44.12 | C |
| ATOM | 6408 | CG | ASN | C | 318 | 46.468 | 45.287 | −11.798 | 1.00 | 44.49 | C |
| ATOM | 6409 | OD1 | ASN | C | 318 | 46.524 | 46.280 | −12.531 | 1.00 | 43.13 | O |
| ATOM | 6410 | ND2 | ASN | C | 318 | 45.502 | 44.367 | −11.896 | 1.00 | 32.86 | N |
| ATOM | 6411 | N | SER | C | 319 | 45.961 | 44.407 | −8.047 | 1.00 | 29.61 | N |
| ATOM | 6412 | CA | SER | C | 319 | 44.733 | 43.894 | −7.448 | 1.00 | 31.50 | C |
| ATOM | 6413 | C | SER | C | 319 | 44.035 | 44.889 | −6.533 | 1.00 | 41.64 | C |
| ATOM | 6414 | O | SER | C | 319 | 42.878 | 44.683 | −6.156 | 1.00 | 39.95 | O |
| ATOM | 6415 | CB | SER | C | 319 | 45.004 | 42.616 | −6.669 | 1.00 | 32.77 | C |
| ATOM | 6416 | OG | SER | C | 319 | 45.209 | 41.533 | −7.545 | 1.00 | 38.85 | O |
| ATOM | 6417 | N | ALA | C | 320 | 44.732 | 45.962 | −6.172 | 1.00 | 42.38 | N |
| ATOM | 6418 | CA | ALA | C | 320 | 44.130 | 46.982 | −5.323 | 1.00 | 40.79 | C |
| ATOM | 6419 | C | ALA | C | 320 | 43.740 | 48.234 | −6.111 | 1.00 | 42.66 | C |
| ATOM | 6420 | O | ALA | C | 320 | 43.017 | 49.094 | −5.612 | 1.00 | 48.99 | O |
| ATOM | 6421 | CB | ALA | C | 320 | 45.054 | 47.331 | −4.176 | 1.00 | 36.59 | C |
| ATOM | 6422 | N | MET | C | 321 | 44.204 | 48.328 | −7.348 | 1.00 | 38.55 | N |
| ATOM | 6423 | CA | MET | C | 321 | 43.937 | 49.512 | −8.159 | 1.00 | 47.66 | C |
| ATOM | 6424 | C | MET | C | 321 | 42.516 | 49.603 | −8.718 | 1.00 | 45.04 | C |
| ATOM | 6425 | O | MET | C | 321 | 41.990 | 50.699 | −8.898 | 1.00 | 48.89 | O |
| ATOM | 6426 | CB | MET | C | 321 | 44.980 | 49.641 | −9.267 | 1.00 | 40.75 | C |
| ATOM | 6427 | CG | MET | C | 321 | 46.356 | 49.919 | −8.703 | 1.00 | 49.11 | C |
| ATOM | 6428 | SD | MET | C | 321 | 47.714 | 49.696 | −9.856 | 1.00 | 60.69 | S |
| ATOM | 6429 | CE | MET | C | 321 | 49.137 | 49.802 | −8.741 | 1.00 | 42.22 | C |
| ATOM | 6430 | N | ASN | C | 322 | 41.894 | 48.459 | −8.976 | 1.00 | 50.35 | N |
| ATOM | 6431 | CA | ASN | C | 322 | 40.553 | 48.436 | −9.556 | 1.00 | 50.38 | C |
| ATOM | 6432 | C | ASN | C | 322 | 39.507 | 49.217 | −8.771 | 1.00 | 52.89 | C |
| ATOM | 6433 | O | ASN | C | 322 | 38.892 | 50.138 | −9.309 | 1.00 | 52.50 | O |
| ATOM | 6434 | CB | ASN | C | 322 | 40.070 | 47.002 | −9.757 | 1.00 | 54.32 | C |
| ATOM | 6435 | CG | ASN | C | 322 | 40.511 | 46.435 | −11.067 | 1.00 | 60.06 | C |
| ATOM | 6436 | OD1 | ASN | C | 322 | 41.326 | 47.041 | −11.765 | 1.00 | 40.84 | O |
| ATOM | 6437 | ND2 | ASN | C | 322 | 39.973 | 45.268 | −11.423 | 1.00 | 61.97 | N |
| ATOM | 6438 | N | PRO | C | 323 | 39.283 | 48.838 | −7.501 | 1.00 | 52.30 | N |
| ATOM | 6439 | CA | PRO | C | 323 | 38.245 | 49.538 | −6.747 | 1.00 | 51.33 | C |
| ATOM | 6440 | C | PRO | C | 323 | 38.534 | 51.027 | −6.711 | 1.00 | 47.59 | C |
| ATOM | 6441 | O | PRO | C | 323 | 37.605 | 51.818 | −6.834 | 1.00 | 50.18 | O |
| ATOM | 6442 | CB | PRO | C | 323 | 38.348 | 48.915 | −5.351 | 1.00 | 46.35 | C |
| ATOM | 6443 | CG | PRO | C | 323 | 38.894 | 47.547 | −5.601 | 1.00 | 45.73 | C |
| ATOM | 6444 | CD | PRO | C | 323 | 39.892 | 47.753 | −6.710 | 1.00 | 51.47 | C |
| ATOM | 6445 | N | ILE | C | 324 | 39.803 | 51.401 | −6.579 | 1.00 | 44.72 | N |
| ATOM | 6446 | CA | ILE | C | 324 | 40.183 | 52.809 | −6.646 | 1.00 | 51.59 | C |
| ATOM | 6447 | C | ILE | C | 324 | 39.725 | 53.453 | −7.951 | 1.00 | 53.69 | C |
| ATOM | 6448 | O | ILE | C | 324 | 39.089 | 54.502 | −7.941 | 1.00 | 59.30 | O |
| ATOM | 6449 | CB | ILE | C | 324 | 41.700 | 53.002 | −6.524 | 1.00 | 53.74 | C |
| ATOM | 6450 | CG1 | ILE | C | 324 | 42.127 | 52.925 | −5.056 | 1.00 | 52.85 | C |
| ATOM | 6451 | CG2 | ILE | C | 324 | 42.118 | 54.334 | −7.157 | 1.00 | 37.38 | C |
| ATOM | 6452 | CD1 | ILE | C | 324 | 43.607 | 52.669 | −4.878 | 1.00 | 60.65 | C |
| ATOM | 6453 | N | ILE | C | 325 | 40.053 | 52.824 | −9.074 | 1.00 | 51.40 | N |
| ATOM | 6454 | CA | ILE | C | 325 | 39.695 | 53.364 | −10.381 | 1.00 | 49.51 | C |
| ATOM | 6455 | C | ILE | C | 325 | 38.178 | 53.507 | −10.543 | 1.00 | 58.09 | C |
| ATOM | 6456 | O | ILE | C | 325 | 37.694 | 54.471 | −11.135 | 1.00 | 49.44 | O |
| ATOM | 6457 | CB | ILE | C | 325 | 40.260 | 52.495 | −11.527 | 1.00 | 49.16 | C |
| ATOM | 6458 | CG1 | ILE | C | 325 | 41.779 | 52.411 | −11.431 | 1.00 | 43.52 | C |
| ATOM | 6459 | CG2 | ILE | C | 325 | 39.864 | 53.054 | −12.887 | 1.00 | 49.11 | C |
| ATOM | 6460 | CD1 | ILE | C | 325 | 42.426 | 51.844 | −12.667 | 1.00 | 40.91 | C |
| ATOM | 6461 | N | TYR | C | 326 | 37.426 | 52.553 | −10.009 | 1.00 | 57.77 | N |
| ATOM | 6462 | CA | TYR | C | 326 | 35.977 | 52.608 | −10.118 | 1.00 | 58.32 | C |
| ATOM | 6463 | C | TYR | C | 326 | 35.394 | 53.863 | −9.470 | 1.00 | 60.88 | C |
| ATOM | 6464 | O | TYR | C | 326 | 34.243 | 54.207 | −9.712 | 1.00 | 55.76 | O |
| ATOM | 6465 | CB | TYR | C | 326 | 35.338 | 51.358 | −9.519 | 1.00 | 49.71 | C |
| ATOM | 6466 | CG | TYR | C | 326 | 35.655 | 50.085 | −10.265 | 1.00 | 55.77 | C |
| ATOM | 6467 | CD1 | TYR | C | 326 | 35.747 | 48.873 | −9.591 | 1.00 | 55.96 | C |
| ATOM | 6468 | CD2 | TYR | C | 326 | 35.872 | 50.094 | −11.644 | 1.00 | 54.13 | C |
| ATOM | 6469 | CE1 | TYR | C | 326 | 36.031 | 47.702 | −10.264 | 1.00 | 54.31 | C |
| ATOM | 6470 | CE2 | TYR | C | 326 | 36.162 | 48.926 | −12.330 | 1.00 | 51.16 | C |
| ATOM | 6471 | CZ | TYR | C | 326 | 36.241 | 47.731 | −11.631 | 1.00 | 52.74 | C |
| ATOM | 6472 | OH | TYR | C | 326 | 36.530 | 46.556 | −12.285 | 1.00 | 41.98 | O |
| ATOM | 6473 | N | CYS | C | 327 | 36.194 | 54.544 | −8.655 | 1.00 | 62.46 | N |
| ATOM | 6474 | CA | CYS | C | 327 | 35.757 | 55.773 | −7.989 | 1.00 | 67.59 | C |
| ATOM | 6475 | C | CYS | C | 327 | 35.490 | 56.909 | −8.972 | 1.00 | 74.14 | C |
| ATOM | 6476 | O | CYS | C | 327 | 35.104 | 58.007 | −8.571 | 1.00 | 78.91 | O |
| ATOM | 6477 | CB | CYS | C | 327 | 36.791 | 56.229 | −6.958 | 1.00 | 61.95 | C |
| ATOM | 6478 | SG | CYS | C | 327 | 36.960 | 55.148 | −5.520 | 1.00 | 66.64 | S |
| ATOM | 6479 | N | ARG | C | 328 | 35.709 | 56.653 | −10.256 | 1.00 | 70.02 | N |
| ATOM | 6480 | CA | ARG | C | 328 | 35.417 | 57.649 | −11.279 | 1.00 | 72.23 | C |
| ATOM | 6481 | C | ARG | C | 328 | 33.912 | 57.799 | −11.460 | 1.00 | 82.47 | C |
| ATOM | 6482 | O | ARG | C | 328 | 33.416 | 58.888 | −11.750 | 1.00 | 96.14 | O |
| ATOM | 6483 | CB | ARG | C | 328 | 36.061 | 57.261 | −12.608 | 1.00 | 70.19 | C |
| ATOM | 6484 | CG | ARG | C | 328 | 37.518 | 57.660 | −12.746 | 1.00 | 71.53 | C |
| ATOM | 6485 | CD | ARG | C | 328 | 38.093 | 57.029 | −13.989 | 1.00 | 62.36 | C |
| ATOM | 6486 | NE | ARG | C | 328 | 37.070 | 56.919 | −15.021 | 1.00 | 62.38 | N |
| ATOM | 6487 | CZ | ARG | C | 328 | 37.013 | 57.695 | −16.096 | 1.00 | 75.27 | C |
| ATOM | 6488 | NH1 | ARG | C | 328 | 37.933 | 58.635 | −16.283 | 1.00 | 76.75 | N |
| ATOM | 6489 | NH2 | ARG | C | 328 | 36.041 | 57.528 | −16.988 | 1.00 | 75.44 | N |
| ATOM | 6490 | N | SER | C | 329 | 33.189 | 56.697 | −11.283 | 1.00 | 86.41 | N |
| ATOM | 6491 | CA | SER | C | 329 | 31.749 | 56.675 | −11.519 | 1.00 | 87.97 | C |
| ATOM | 6492 | C | SER | C | 329 | 30.988 | 57.335 | −10.380 | 1.00 | 89.08 | C |
| ATOM | 6493 | O | SER | C | 329 | 31.348 | 57.187 | −9.214 | 1.00 | 73.68 | O |
| ATOM | 6494 | CB | SER | C | 329 | 31.255 | 55.239 | −11.721 | 1.00 | 91.47 | C |
| ATOM | 6495 | OG | SER | C | 329 | 29.872 | 55.211 | −12.026 | 1.00 | 78.47 | O |
| ATOM | 6496 | C16 | PDL | C | 400 | 57.280 | 31.530 | −16.048 | 1.00 | 48.57 | C |
| ATOM | 6497 | N3 | PDL | C | 400 | 58.322 | 31.155 | −16.325 | 1.00 | 40.11 | N |
| ATOM | 6498 | N1 | PDL | C | 400 | 54.713 | 31.580 | −16.545 | 1.00 | 44.31 | N |
| ATOM | 6499 | C1 | PDL | C | 400 | 55.943 | 31.977 | −15.752 | 1.00 | 46.12 | C |
| ATOM | 6500 | C2 | PDL | C | 400 | 55.507 | 32.968 | −14.687 | 1.00 | 32.90 | C |
| ATOM | 6501 | C3 | PDL | C | 400 | 54.028 | 33.125 | −14.796 | 1.00 | 28.37 | C |
| ATOM | 6502 | C4 | PDL | C | 400 | 53.008 | 33.944 | −14.009 | 1.00 | 28.86 | C |
| ATOM | 6503 | C5 | PDL | C | 400 | 51.521 | 33.904 | −14.358 | 1.00 | 34.03 | C |
| ATOM | 6504 | C6 | PDL | C | 400 | 51.053 | 33.025 | −15.526 | 1.00 | 31.57 | C |
| ATOM | 6505 | C7 | PDL | C | 400 | 52.053 | 32.203 | −16.319 | 1.00 | 21.76 | C |
| ATOM | 6506 | C8 | PDL | C | 400 | 53.539 | 32.264 | −15.955 | 1.00 | 31.07 | C |
| ATOM | 6507 | O1 | PDL | C | 400 | 53.447 | 34.772 | −12.978 | 1.00 | 39.15 | O |
| ATOM | 6508 | C9 | PDL | C | 400 | 52.668 | 34.964 | −11.833 | 1.00 | 36.42 | C |
| ATOM | 6509 | C10 | PDL | C | 400 | 53.617 | 35.479 | −10.745 | 1.00 | 29.67 | C |
| ATOM | 6510 | O2 | PDL | C | 400 | 52.918 | 36.329 | −9.865 | 1.00 | 37.36 | O |
| ATOM | 6511 | C11 | PDL | C | 400 | 54.253 | 34.287 | −10.010 | 1.00 | 30.49 | C |
| ATOM | 6512 | N2 | PDL | C | 400 | 54.979 | 34.620 | −8.778 | 1.00 | 26.38 | N |
| ATOM | 6513 | C12 | PDL | C | 400 | 55.824 | 33.468 | −8.414 | 1.00 | 31.75 | C |
| ATOM | 6514 | C13 | PDL | C | 400 | 57.094 | 33.491 | −9.314 | 1.00 | 19.60 | C |
| ATOM | 6515 | C14 | PDL | C | 400 | 55.087 | 32.110 | −8.630 | 1.00 | 21.33 | C |
| ATOM | 6516 | C15 | PDL | C | 400 | 56.168 | 33.605 | −6.908 | 1.00 | 29.02 | C |
| ATOM | 6517 | NA | NA | C | 401 | 61.282 | 23.554 | −2.478 | 1.00 | 37.52 | Na |

TABLE D
CRYST1 55.500 86.800 95.500 67.60 73.30 85.80 P 1
SCALE1 0.018018 −0.001323 −0.005298 0.00000
SCALE2 0.000000 0.011552 −0.004700 0.00000
SCALE3 0.000000 0.000000 0.011803 0.00000

| | | | | | | | | | | | |
|---|---|---|---|---|---|---|---|---|---|---|---|
| ATOM | 6518 | N | GLU | D | 33 | 32.936 | 12.310 | −58.825 | 1.00 | 70.87 | N |
| ATOM | 6519 | CA | GLU | D | 33 | 32.400 | 11.204 | −58.046 | 1.00 | 67.67 | C |
| ATOM | 6520 | C | GLU | D | 33 | 33.262 | 9.953 | −58.194 | 1.00 | 63.61 | C |
| ATOM | 6521 | O | GLU | D | 33 | 33.563 | 9.283 | −57.206 | 1.00 | 56.75 | O |
| ATOM | 6522 | CB | GLU | D | 33 | 30.956 | 10.915 | −58.442 | 1.00 | 59.28 | C |
| ATOM | 6523 | CG | GLU | D | 33 | 30.241 | 10.019 | −57.452 | 1.00 | 80.84 | C |
| ATOM | 6524 | CD | GLU | D | 33 | 28.927 | 9.491 | −57.985 | 1.00 | 95.18 | C |
| ATOM | 6525 | OE1 | GLU | D | 33 | 28.516 | 9.909 | −59.092 | 1.00 | 100.08 | O |
| ATOM | 6526 | OE2 | GLU | D | 33 | 28.309 | 8.652 | −57.294 | 1.00 | 85.89 | O |
| ATOM | 6527 | N | ALA | D | 34 | 33.652 | 9.634 | −59.426 | 1.00 | 61.18 | N |
| ATOM | 6528 | CA | ALA | D | 34 | 34.650 | 8.600 | −59.641 | 1.00 | 55.63 | C |
| ATOM | 6529 | C | ALA | D | 34 | 35.905 | 9.021 | −58.883 | 1.00 | 56.94 | C |
| ATOM | 6530 | O | ALA | D | 34 | 36.497 | 8.231 | −58.141 | 1.00 | 51.45 | O |
| ATOM | 6531 | CB | ALA | D | 34 | 34.945 | 8.428 | −61.119 | 1.00 | 33.86 | C |
| ATOM | 6532 | N | GLY | D | 35 | 36.295 | 10.280 | −59.056 | 1.00 | 51.21 | N |
| ATOM | 6533 | CA | GLY | D | 35 | 37.450 | 10.820 | −58.357 | 1.00 | 53.13 | C |
| ATOM | 6534 | C | GLY | D | 35 | 37.378 | 10.669 | −56.846 | 1.00 | 49.57 | C |
| ATOM | 6535 | O | GLY | D | 35 | 38.199 | 9.989 | −56.236 | 1.00 | 44.13 | O |
| ATOM | 6536 | N | MET | D | 36 | 36.388 | 11.304 | −56.233 | 1.00 | 56.71 | N |
| ATOM | 6537 | CA | MET | D | 36 | 36.237 | 11.211 | −54.788 | 1.00 | 45.41 | C |
| ATOM | 6538 | C | MET | D | 36 | 36.219 | 9.760 | −54.314 | 1.00 | 44.98 | C |
| ATOM | 6539 | O | MET | D | 36 | 36.895 | 9.410 | −53.353 | 1.00 | 47.98 | O |
| ATOM | 6540 | CB | MET | D | 36 | 34.994 | 11.969 | −54.321 | 1.00 | 51.72 | C |
| ATOM | 6541 | CG | MET | D | 36 | 35.112 | 13.489 | −54.472 | 1.00 | 67.06 | C |
| ATOM | 6542 | SD | MET | D | 36 | 36.558 | 14.224 | −53.653 | 1.00 | 85.95 | S |
| ATOM | 6543 | CE | MET | D | 36 | 37.873 | 14.004 | −54.865 | 1.00 | 55.31 | C |
| ATOM | 6544 | N | SER | D | 37 | 35.459 | 8.915 | −55.001 | 1.00 | 46.54 | N |
| ATOM | 6545 | CA | SER | D | 37 | 35.384 | 7.499 | −54.659 | 1.00 | 43.19 | C |
| ATOM | 6546 | C | SER | D | 37 | 36.760 | 6.834 | −54.607 | 1.00 | 40.39 | C |
| ATOM | 6547 | O | SER | D | 37 | 37.055 | 6.062 | −53.687 | 1.00 | 30.12 | O |
| ATOM | 6548 | CB | SER | D | 37 | 34.481 | 6.754 | −55.645 | 1.00 | 45.51 | C |
| ATOM | 6549 | OG | SER | D | 37 | 33.127 | 7.117 | −55.460 | 1.00 | 49.21 | O |
| ATOM | 6550 | N | LEU | D | 38 | 37.595 | 7.121 | −55.600 | 1.00 | 33.21 | N |
| ATOM | 6551 | CA | LEU | D | 38 | 38.911 | 6.508 | −55.653 | 1.00 | 38.92 | C |
| ATOM | 6552 | C | LEU | D | 38 | 39.798 | 7.011 | −54.500 | 1.00 | 44.71 | C |
| ATOM | 6553 | O | LEU | D | 38 | 40.376 | 6.217 | −53.741 | 1.00 | 34.90 | O |
| ATOM | 6554 | CB | LEU | D | 38 | 39.573 | 6.751 | −57.007 | 1.00 | 32.16 | C |
| ATOM | 6555 | CG | LEU | D | 38 | 40.888 | 6.005 | −57.200 | 1.00 | 38.54 | C |
| ATOM | 6556 | CD1 | LEU | D | 38 | 40.674 | 4.490 | −57.182 | 1.00 | 38.61 | C |
| ATOM | 6557 | CD2 | LEU | D | 38 | 41.544 | 6.443 | −58.483 | 1.00 | 37.96 | C |
| ATOM | 6558 | N | LEU | D | 39 | 39.896 | 8.328 | −54.363 | 1.00 | 34.73 | N |
| ATOM | 6559 | CA | LEU | D | 39 | 40.613 | 8.909 | −53.235 | 1.00 | 41.35 | C |
| ATOM | 6560 | C | LEU | D | 39 | 40.246 | 8.249 | −51.893 | 1.00 | 39.85 | C |
| ATOM | 6561 | O | LEU | D | 39 | 41.123 | 7.917 | −51.099 | 1.00 | 37.81 | O |
| ATOM | 6562 | CB | LEU | D | 39 | 40.367 | 10.418 | −53.152 | 1.00 | 46.88 | C |
| ATOM | 6563 | CG | LEU | D | 39 | 41.128 | 11.131 | −52.029 | 1.00 | 42.29 | C |
| ATOM | 6564 | CD1 | LEU | D | 39 | 42.630 | 11.197 | −52.335 | 1.00 | 37.23 | C |
| ATOM | 6565 | CD2 | LEU | D | 39 | 40.559 | 12.524 | −51.800 | 1.00 | 51.38 | C |
| ATOM | 6566 | N | MET | D | 40 | 38.956 | 8.056 | −51.644 | 1.00 | 35.09 | N |
| ATOM | 6567 | CA | MET | D | 40 | 38.503 | 7.427 | −50.406 | 1.00 | 28.75 | C |
| ATOM | 6568 | C | MET | D | 40 | 38.929 | 5.966 | −50.266 | 1.00 | 32.63 | C |
| ATOM | 6569 | O | MET | D | 40 | 39.318 | 5.542 | −49.191 | 1.00 | 32.67 | O |
| ATOM | 6570 | CB | MET | D | 40 | 36.983 | 7.567 | −50.244 | 1.00 | 40.54 | C |
| ATOM | 6571 | CG | MET | D | 40 | 36.493 | 9.015 | −50.079 | 1.00 | 55.55 | C |
| ATOM | 6572 | SD | MET | D | 40 | 37.179 | 9.921 | −48.656 | 1.00 | 81.76 | S |
| ATOM | 6573 | CE | MET | D | 40 | 38.693 | 10.617 | −49.329 | 1.00 | 44.42 | C |
| ATOM | 6574 | N | ALA | D | 41 | 38.869 | 5.191 | −51.345 | 1.00 | 42.14 | N |
| ATOM | 6575 | CA | ALA | D | 41 | 39.361 | 3.818 | −51.301 | 1.00 | 29.34 | C |
| ATOM | 6576 | C | ALA | D | 41 | 40.883 | 3.802 | −51.154 | 1.00 | 37.62 | C |
| ATOM | 6577 | O | ALA | D | 41 | 41.483 | 2.761 | −50.894 | 1.00 | 37.37 | O |
| ATOM | 6578 | CB | ALA | D | 41 | 38.942 | 3.070 | −52.549 | 1.00 | 35.84 | C |
| ATOM | 6579 | N | LEU | D | 42 | 41.502 | 4.969 | −51.305 | 1.00 | 35.43 | N |
| ATOM | 6580 | CA | LEU | D | 42 | 42.958 | 5.082 | −51.316 | 1.00 | 35.07 | C |
| ATOM | 6581 | C | LEU | D | 42 | 43.597 | 5.793 | −50.102 | 1.00 | 36.06 | C |
| ATOM | 6582 | O | LEU | D | 42 | 44.818 | 5.680 | −49.923 | 1.00 | 31.94 | O |
| ATOM | 6583 | CB | LEU | D | 42 | 43.415 | 5.765 | −52.625 | 1.00 | 43.14 | C |
| ATOM | 6584 | CG | LEU | D | 42 | 44.198 | 5.014 | −53.720 | 1.00 | 32.16 | C |
| ATOM | 6585 | CD1 | LEU | D | 42 | 44.049 | 3.520 | −53.616 | 1.00 | 28.71 | C |
| ATOM | 6586 | CD2 | LEU | D | 42 | 43.793 | 5.481 | −55.092 | 1.00 | 24.33 | C |
| ATOM | 6587 | N | VAL | D | 43 | 42.807 | 6.514 | −49.290 | 1.00 | 27.82 | N |
| ATOM | 6588 | CA | VAL | D | 43 | 43.364 | 7.382 | −48.219 | 1.00 | 30.44 | C |
| ATOM | 6589 | C | VAL | D | 43 | 44.268 | 6.738 | −47.181 | 1.00 | 31.15 | C |
| ATOM | 6590 | O | VAL | D | 43 | 45.344 | 7.257 | −46.915 | 1.00 | 31.06 | O |
| ATOM | 6591 | CB | VAL | D | 43 | 42.309 | 8.167 | −47.393 | 1.00 | 30.55 | C |
| ATOM | 6592 | CG1 | VAL | D | 43 | 42.385 | 9.663 | −47.694 | 1.00 | 33.24 | C |
| ATOM | 6593 | CG2 | VAL | D | 43 | 40.911 | 7.590 | −47.567 | 1.00 | 44.75 | C |
| ATOM | 6594 | N | VAL | D | 44 | 43.821 | 5.652 | −46.552 | 1.00 | 28.40 | N |
| ATOM | 6595 | CA | VAL | D | 44 | 44.656 | 5.004 | −45.547 | 1.00 | 26.07 | C |
| ATOM | 6596 | C | VAL | D | 44 | 46.028 | 4.706 | −46.122 | 1.00 | 32.39 | C |
| ATOM | 6597 | O | VAL | D | 44 | 47.048 | 4.985 | −45.488 | 1.00 | 34.61 | O |
| ATOM | 6598 | CB | VAL | D | 44 | 44.038 | 3.720 | −45.000 | 1.00 | 30.89 | C |
| ATOM | 6599 | CG1 | VAL | D | 44 | 45.029 | 3.005 | −44.085 | 1.00 | 19.55 | C |
| ATOM | 6600 | CG2 | VAL | D | 44 | 42.753 | 4.036 | −44.253 | 1.00 | 31.89 | C |
| ATOM | 6601 | N | LEU | D | 45 | 46.051 | 4.155 | −47.332 | 1.00 | 34.18 | N |
| ATOM | 6602 | CA | LEU | D | 45 | 47.310 | 3.907 | −48.023 | 1.00 | 33.44 | C |
| ATOM | 6603 | C | LEU | D | 45 | 48.062 | 5.218 | −48.276 | 1.00 | 34.22 | C |
| ATOM | 6604 | O | LEU | D | 45 | 49.210 | 5.369 | −47.850 | 1.00 | 33.31 | O |
| ATOM | 6605 | CB | LEU | D | 45 | 47.079 | 3.152 | −49.342 | 1.00 | 29.70 | C |
| ATOM | 6606 | CG | LEU | D | 45 | 48.343 | 2.980 | −50.196 | 1.00 | 34.73 | C |
| ATOM | 6607 | CD1 | LEU | D | 45 | 49.286 | 1.896 | −49.644 | 1.00 | 23.71 | C |
| ATOM | 6608 | CD2 | LEU | D | 45 | 47.969 | 2.703 | −51.636 | 1.00 | 33.78 | C |
| ATOM | 6609 | N | LEU | D | 46 | 47.404 | 6.157 | −48.958 | 1.00 | 29.97 | N |
| ATOM | 6610 | CA | LEU | D | 46 | 47.993 | 7.460 | −49.289 | 1.00 | 32.71 | C |
| ATOM | 6611 | C | LEU | D | 46 | 48.594 | 8.243 | −48.110 | 1.00 | 37.56 | C |
| ATOM | 6612 | O | LEU | D | 46 | 49.624 | 8.908 | −48.260 | 1.00 | 40.65 | O |
| ATOM | 6613 | CB | LEU | D | 46 | 46.966 | 8.338 | −50.002 | 1.00 | 31.94 | C |
| ATOM | 6614 | CG | LEU | D | 46 | 46.673 | 8.005 | −51.457 | 1.00 | 37.27 | C |
| ATOM | 6615 | CD1 | LEU | D | 46 | 45.775 | 9.073 | −52.071 | 1.00 | 37.01 | C |
| ATOM | 6616 | CD2 | LEU | D | 46 | 47.976 | 7.900 | −52.211 | 1.00 | 28.57 | C |
| ATOM | 6617 | N | ILE | D | 47 | 47.934 | 8.189 | −46.956 | 1.00 | 33.73 | N |
| ATOM | 6618 | CA | ILE | D | 47 | 48.418 | 8.881 | −45.765 | 1.00 | 36.23 | C |
| ATOM | 6619 | C | ILE | D | 47 | 49.607 | 8.147 | −45.149 | 1.00 | 37.47 | C |
| ATOM | 6620 | O | ILE | D | 47 | 50.623 | 8.764 | −44.840 | 1.00 | 31.91 | O |
| ATOM | 6621 | CB | ILE | D | 47 | 47.308 | 9.055 | −44.688 | 1.00 | 31.27 | C |
| ATOM | 6622 | CG1 | ILE | D | 47 | 46.215 | 10.005 | −45.172 | 1.00 | 32.52 | C |
| ATOM | 6623 | CG2 | ILE | D | 47 | 47.888 | 9.587 | −43.413 | 1.00 | 25.53 | C |
| ATOM | 6624 | CD1 | ILE | D | 47 | 44.922 | 9.909 | −44.370 | 1.00 | 28.45 | C |
| ATOM | 6625 | N | VAL | D | 48 | 49.473 | 6.830 | −44.978 | 1.00 | 42.08 | N |
| ATOM | 6626 | CA | VAL | D | 48 | 50.486 | 6.021 | −44.286 | 1.00 | 32.68 | C |
| ATOM | 6627 | C | VAL | D | 48 | 51.755 | 5.792 | −45.097 | 1.00 | 31.83 | C |
| ATOM | 6628 | O | VAL | D | 48 | 52.849 | 6.093 | −44.630 | 1.00 | 36.80 | O |
| ATOM | 6629 | CB | VAL | D | 48 | 49.930 | 4.654 | −43.824 | 1.00 | 30.07 | C |
| ATOM | 6630 | CG1 | VAL | D | 48 | 51.056 | 3.761 | −43.350 | 1.00 | 22.65 | C |
| ATOM | 6631 | CG2 | VAL | D | 48 | 48.912 | 4.845 | −42.723 | 1.00 | 27.78 | C |
| ATOM | 6632 | N | ALA | D | 49 | 51.616 | 5.248 | −46.300 | 1.00 | 35.77 | N |
| ATOM | 6633 | CA | ALA | D | 49 | 52.777 | 5.006 | −47.155 | 1.00 | 38.62 | C |
| ATOM | 6634 | C | ALA | D | 49 | 53.495 | 6.310 | −47.509 | 1.00 | 40.22 | C |
| ATOM | 6635 | O | ALA | D | 49 | 54.725 | 6.359 | −47.570 | 1.00 | 37.53 | O |
| ATOM | 6636 | CB | ALA | D | 49 | 52.363 | 4.276 | −48.411 | 1.00 | 30.17 | C |
| ATOM | 6637 | N | GLY | D | 50 | 52.718 | 7.366 | −47.728 | 1.00 | 39.60 | N |
| ATOM | 6638 | CA | GLY | D | 50 | 53.263 | 8.651 | −48.131 | 1.00 | 37.20 | C |
| ATOM | 6639 | C | GLY | D | 50 | 53.988 | 9.403 | −47.030 | 1.00 | 38.82 | C |
| ATOM | 6640 | O | GLY | D | 50 | 54.955 | 10.109 | −47.282 | 1.00 | 37.88 | O |
| ATOM | 6641 | N | ASN | D | 51 | 53.519 | 9.264 | −45.799 | 1.00 | 33.65 | N |
| ATOM | 6642 | CA | ASN | D | 51 | 54.159 | 9.957 | −44.694 | 1.00 | 31.72 | C |
| ATOM | 6643 | C | ASN | D | 51 | 55.291 | 9.134 | −44.130 | 1.00 | 31.26 | C |
| ATOM | 6644 | O | ASN | D | 51 | 56.282 | 9.681 | −43.685 | 1.00 | 36.48 | O |
| ATOM | 6645 | CB | ASN | D | 51 | 53.145 | 10.330 | −43.608 | 1.00 | 29.98 | C |
| ATOM | 6646 | CG | ASN | D | 51 | 52.324 | 11.555 | −43.983 | 1.00 | 37.23 | C |
| ATOM | 6647 | OD1 | ASN | D | 51 | 52.764 | 12.697 | −43.792 | 1.00 | 26.37 | O |
| ATOM | 6648 | ND2 | ASN | D | 51 | 51.120 | 11.323 | −44.527 | 1.00 | 29.89 | N |
| ATOM | 6649 | N | VAL | D | 52 | 55.136 | 7.816 | −44.147 | 1.00 | 30.02 | N |
| ATOM | 6650 | CA | VAL | D | 52 | 56.237 | 6.929 | −43.834 | 1.00 | 29.40 | C |
| ATOM | 6651 | C | VAL | D | 52 | 57.365 | 7.238 | −44.800 | 1.00 | 37.99 | C |
| ATOM | 6652 | O | VAL | D | 52 | 58.538 | 7.177 | −44.447 | 1.00 | 32.51 | O |
| ATOM | 6653 | CB | VAL | D | 52 | 55.840 | 5.454 | −43.986 | 1.00 | 36.39 | C |
| ATOM | 6654 | CG1 | VAL | D | 52 | 57.076 | 4.585 | −44.201 | 1.00 | 21.30 | C |
| ATOM | 6655 | CG2 | VAL | D | 52 | 55.045 | 4.983 | −42.760 | 1.00 | 33.79 | C |
| ATOM | 6656 | N | LEU | D | 53 | 56.994 | 7.598 | −46.023 | 1.00 | 44.61 | N |
| ATOM | 6657 | CA | LEU | D | 53 | 57.968 | 7.879 | −47.071 | 1.00 | 43.38 | C |
| ATOM | 6658 | C | LEU | D | 53 | 58.694 | 9.214 | −46.853 | 1.00 | 43.93 | C |
| ATOM | 6659 | O | LEU | D | 53 | 59.896 | 9.311 | −47.072 | 1.00 | 42.30 | O |
| ATOM | 6660 | CB | LEU | D | 53 | 57.289 | 7.847 | −48.437 | 1.00 | 38.10 | C |
| ATOM | 6661 | CG | LEU | D | 53 | 58.097 | 7.179 | −49.545 | 1.00 | 67.46 | C |
| ATOM | 6662 | CD1 | LEU | D | 53 | 58.424 | 5.729 | −49.189 | 1.00 | 53.95 | C |
| ATOM | 6663 | CD2 | LEU | D | 53 | 57.335 | 7.255 | −50.857 | 1.00 | 79.07 | C |
| ATOM | 6664 | N | VAL | D | 54 | 57.957 | 10.234 | −46.418 | 1.00 | 46.27 | N |
| ATOM | 6665 | CA | VAL | D | 54 | 58.547 | 11.533 | −46.107 | 1.00 | 37.90 | C |
| ATOM | 6666 | C | VAL | D | 54 | 59.530 | 11.374 | −44.959 | 1.00 | 40.66 | C |
| ATOM | 6667 | O | VAL | D | 54 | 60.641 | 11.897 | −44.985 | 1.00 | 40.02 | O |
| ATOM | 6668 | CB | VAL | D | 54 | 57.472 | 12.571 | −45.708 | 1.00 | 38.03 | C |
| ATOM | 6669 | CG1 | VAL | D | 54 | 58.108 | 13.751 | −44.994 | 1.00 | 37.10 | C |
| ATOM | 6670 | CG2 | VAL | D | 54 | 56.678 | 13.043 | −46.928 | 1.00 | 34.18 | C |
| ATOM | 6671 | N | ILE | D | 55 | 59.109 | 10.636 | −43.943 | 1.00 | 41.45 | N |
| ATOM | 6672 | CA | ILE | D | 55 | 59.953 | 10.377 | −42.788 | 1.00 | 44.15 | C |
| ATOM | 6673 | C | ILE | D | 55 | 61.257 | 9.691 | −43.207 | 1.00 | 51.35 | C |
| ATOM | 6674 | O | ILE | D | 55 | 62.336 | 10.060 | −42.745 | 1.00 | 60.92 | O |
| ATOM | 6675 | CB | ILE | D | 55 | 59.192 | 9.553 | −41.718 | 1.00 | 35.28 | C |
| ATOM | 6676 | CG1 | ILE | D | 55 | 58.372 | 10.480 | −40.820 | 1.00 | 29.86 | C |
| ATOM | 6677 | CG2 | ILE | D | 55 | 60.136 | 8.731 | −40.887 | 1.00 | 26.01 | C |
| ATOM | 6678 | CD1 | ILE | D | 55 | 57.358 | 9.750 | −39.981 | 1.00 | 33.25 | C |
| ATOM | 6679 | N | ALA | D | 56 | 61.168 | 8.715 | −44.101 | 1.00 | 49.27 | N |
| ATOM | 6680 | CA | ALA | D | 56 | 62.352 | 7.968 | −44.503 | 1.00 | 41.51 | C |
| ATOM | 6681 | C | ALA | D | 56 | 63.265 | 8.805 | −45.391 | 1.00 | 44.77 | C |
| ATOM | 6682 | O | ALA | D | 56 | 64.479 | 8.723 | −45.282 | 1.00 | 57.62 | O |
| ATOM | 6683 | CB | ALA | D | 56 | 61.961 | 6.669 | −45.199 | 1.00 | 41.32 | C |
| ATOM | 6684 | N | ALA | D | 57 | 62.685 | 9.602 | −46.276 | 1.00 | 40.32 | N |
| ATOM | 6685 | CA | ALA | D | 57 | 63.481 | 10.455 | −47.145 | 1.00 | 45.09 | C |
| ATOM | 6686 | C | ALA | D | 57 | 64.287 | 11.448 | −46.316 | 1.00 | 51.73 | C |
| ATOM | 6687 | O | ALA | D | 57 | 65.493 | 11.592 | −46.495 | 1.00 | 56.72 | O |
| ATOM | 6688 | CB | ALA | D | 57 | 62.588 | 11.185 | −48.156 | 1.00 | 38.80 | C |
| ATOM | 6689 | N | ILE | D | 58 | 63.611 | 12.131 | −45.402 | 1.00 | 51.31 | N |
| ATOM | 6690 | CA | ILE | D | 58 | 64.264 | 13.101 | −44.538 | 1.00 | 52.64 | C |
| ATOM | 6691 | C | ILE | D | 58 | 65.345 | 12.422 | −43.710 | 1.00 | 52.46 | C |
| ATOM | 6692 | O | ILE | D | 58 | 66.323 | 13.051 | −43.319 | 1.00 | 69.02 | O |
| ATOM | 6693 | CB | ILE | D | 58 | 63.246 | 13.797 | −43.603 | 1.00 | 50.59 | C |
| ATOM | 6694 | CG1 | ILE | D | 58 | 62.404 | 14.810 | −44.379 | 1.00 | 41.95 | C |
| ATOM | 6695 | CG2 | ILE | D | 58 | 63.950 | 14.490 | −42.448 | 1.00 | 50.05 | C |
| ATOM | 6696 | CD1 | ILE | D | 58 | 61.272 | 15.405 | −43.570 | 1.00 | 31.71 | C |
| ATOM | 6697 | N | GLY | D | 59 | 65.166 | 11.131 | −43.450 | 1.00 | 50.08 | N |
| ATOM | 6698 | CA | GLY | D | 59 | 66.102 | 10.377 | −42.634 | 1.00 | 56.47 | C |
| ATOM | 6699 | C | GLY | D | 59 | 67.225 | 9.699 | −43.410 | 1.00 | 58.41 | C |
| ATOM | 6700 | O | GLY | D | 59 | 68.112 | 9.096 | −42.820 | 1.00 | 59.21 | O |
| ATOM | 6701 | N | SER | D | 60 | 67.185 | 9.781 | −44.734 | 1.00 | 54.47 | N |
| ATOM | 6702 | CA | SER | D | 60 | 68.256 | 9.240 | −45.558 | 1.00 | 54.43 | C |
| ATOM | 6703 | C | SER | D | 60 | 69.174 | 10.377 | −45.954 | 1.00 | 67.14 | C |
| ATOM | 6704 | O | SER | D | 60 | 70.239 | 10.557 | −45.369 | 1.00 | 84.46 | O |
| ATOM | 6705 | CB | SER | D | 60 | 67.700 | 8.573 | −46.820 | 1.00 | 63.72 | C |
| ATOM | 6706 | OG | SER | D | 60 | 67.110 | 7.319 | −46.534 | 1.00 | 65.60 | O |
| ATOM | 6707 | N | THR | D | 61 | 68.744 | 11.144 | −46.953 | 1.00 | 69.90 | N |
| ATOM | 6708 | CA | THR | D | 61 | 69.473 | 12.318 | −47.422 | 1.00 | 70.17 | C |
| ATOM | 6709 | C | THR | D | 61 | 69.738 | 13.299 | −46.292 | 1.00 | 82.42 | C |
| ATOM | 6710 | O | THR | D | 61 | 68.839 | 14.038 | −45.891 | 1.00 | 83.13 | O |
| ATOM | 6711 | CB | THR | D | 61 | 68.668 | 13.097 | −48.471 | 1.00 | 63.98 | C |
| ATOM | 6712 | OG1 | THR | D | 61 | 67.808 | 12.208 | −49.193 | 1.00 | 69.96 | O |
| ATOM | 6713 | CG2 | THR | D | 61 | 69.601 | 13.816 | −49.433 | 1.00 | 80.94 | C |
| ATOM | 6714 | N | GLN | D | 62 | 70.967 | 13.315 | −45.786 | 1.00 | 92.10 | N |
| ATOM | 6715 | CA | GLN | D | 62 | 71.358 | 14.298 | −44.782 | 1.00 | 97.66 | C |
| ATOM | 6716 | C | GLN | D | 62 | 71.250 | 15.687 | −45.403 | 1.00 | 95.07 | C |
| ATOM | 6717 | O | GLN | D | 62 | 71.073 | 16.693 | −44.708 | 1.00 | 92.30 | O |
| ATOM | 6718 | CB | GLN | D | 62 | 72.774 | 14.012 | −44.289 | 1.00 | 106.21 | C |
| ATOM | 6719 | CG | GLN | D | 62 | 72.878 | 12.691 | −43.539 | 1.00 | 119.48 | C |
| ATOM | 6720 | CD | GLN | D | 62 | 74.182 | 11.969 | −43.796 | 1.00 | 137.39 | C |
| ATOM | 6721 | OE1 | GLN | D | 62 | 75.220 | 12.594 | −44.018 | 1.00 | 146.63 | O |
| ATOM | 6722 | NE2 | GLN | D | 62 | 74.136 | 10.640 | −43.768 | 1.00 | 135.26 | N |
| ATOM | 6723 | N | ARG | D | 63 | 71.341 | 15.713 | −46.729 | 1.00 | 74.98 | N |
| ATOM | 6724 | CA | ARG | D | 63 | 71.112 | 16.909 | −47.523 | 1.00 | 67.77 | C |
| ATOM | 6725 | C | ARG | D | 63 | 69.652 | 17.379 | −47.411 | 1.00 | 80.85 | C |
| ATOM | 6726 | O | ARG | D | 63 | 69.312 | 18.502 | −47.793 | 1.00 | 81.86 | O |
| ATOM | 6727 | CB | ARG | D | 63 | 71.470 | 16.600 | −48.976 | 1.00 | 89.41 | C |
| ATOM | 6728 | CG | ARG | D | 63 | 70.981 | 17.608 | −49.991 | 1.00 | 101.26 | C |
| ATOM | 6729 | CD | ARG | D | 63 | 70.620 | 16.902 | −51.278 | 1.00 | 102.07 | C |
| ATOM | 6730 | NE | ARG | D | 63 | 70.776 | 17.762 | −52.445 | 1.00 | 121.62 | N |
| ATOM | 6731 | CZ | ARG | D | 63 | 70.251 | 17.496 | −53.638 | 1.00 | 125.88 | C |
| ATOM | 6732 | NH1 | ARG | D | 63 | 69.522 | 16.399 | −53.811 | 1.00 | 117.63 | N |
| ATOM | 6733 | NH2 | ARG | D | 63 | 70.445 | 18.327 | −54.654 | 1.00 | 116.52 | N |
| ATOM | 6734 | N | LEU | D | 64 | 68.791 | 16.502 | −46.902 | 1.00 | 78.89 | N |
| ATOM | 6735 | CA | LEU | D | 64 | 67.419 | 16.864 | −46.561 | 1.00 | 67.09 | C |
| ATOM | 6736 | C | LEU | D | 64 | 67.275 | 17.035 | −45.048 | 1.00 | 66.22 | C |
| ATOM | 6737 | O | LEU | D | 64 | 66.176 | 17.225 | −44.538 | 1.00 | 55.43 | O |
| ATOM | 6738 | CB | LEU | D | 64 | 66.436 | 15.795 | −47.039 | 1.00 | 63.31 | C |
| ATOM | 6739 | CG | LEU | D | 64 | 65.783 | 15.905 | −48.419 | 1.00 | 59.21 | C |
| ATOM | 6740 | CD1 | LEU | D | 64 | 64.736 | 14.808 | −48.574 | 1.00 | 47.84 | C |
| ATOM | 6741 | CD2 | LEU | D | 64 | 65.167 | 17.277 | −48.659 | 1.00 | 43.21 | C |
| ATOM | 6742 | N | GLN | D | 65 | 68.384 | 16.952 | −44.326 | 1.00 | 73.42 | N |
| ATOM | 6743 | CA | GLN | D | 65 | 68.340 | 17.148 | −42.880 | 1.00 | 80.63 | C |
| ATOM | 6744 | C | GLN | D | 65 | 68.621 | 18.597 | −42.491 | 1.00 | 72.75 | C |
| ATOM | 6745 | O | GLN | D | 65 | 69.768 | 19.042 | −42.464 | 1.00 | 70.43 | O |
| ATOM | 6746 | CB | GLN | D | 65 | 69.271 | 16.173 | −42.158 | 1.00 | 79.32 | C |
| ATOM | 6747 | CG | GLN | D | 65 | 68.582 | 14.868 | −41.797 | 1.00 | 79.58 | C |
| ATOM | 6748 | CD | GLN | D | 65 | 69.533 | 13.842 | −41.225 | 1.00 | 98.69 | C |
| ATOM | 6749 | OE1 | GLN | D | 65 | 70.667 | 13.709 | −41.687 | 1.00 | 99.45 | O |
| ATOM | 6750 | NE2 | GLN | D | 65 | 69.074 | 13.101 | −40.217 | 1.00 | 94.97 | N |
| ATOM | 6751 | N | THR | D | 66 | 67.545 | 19.326 | −42.212 | 1.00 | 62.88 | N |
| ATOM | 6752 | CA | THR | D | 66 | 67.609 | 20.740 | −41.875 | 1.00 | 50.75 | C |
| ATOM | 6753 | C | THR | D | 66 | 66.583 | 21.023 | −40.783 | 1.00 | 45.40 | C |
| ATOM | 6754 | O | THR | D | 66 | 65.666 | 20.234 | −40.582 | 1.00 | 47.66 | O |
| ATOM | 6755 | CB | THR | D | 66 | 67.321 | 21.626 | −43.109 | 1.00 | 49.94 | C |
| ATOM | 6756 | OG1 | THR | D | 66 | 65.960 | 21.466 | −43.526 | 1.00 | 40.40 | O |
| ATOM | 6757 | CG2 | THR | D | 66 | 68.228 | 21.240 | −44.257 | 1.00 | 49.41 | C |
| ATOM | 6758 | N | LEU | D | 67 | 66.750 | 22.129 | −40.066 | 1.00 | 48.46 | N |
| ATOM | 6759 | CA | LEU | D | 67 | 65.805 | 22.525 | −39.027 | 1.00 | 44.51 | C |
| ATOM | 6760 | C | LEU | D | 67 | 64.356 | 22.433 | −39.493 | 1.00 | 47.00 | C |
| ATOM | 6761 | O | LEU | D | 67 | 63.553 | 21.710 | −38.903 | 1.00 | 44.65 | O |
| ATOM | 6762 | CB | LEU | D | 67 | 66.085 | 23.956 | −38.575 | 1.00 | 56.07 | C |
| ATOM | 6763 | CG | LEU | D | 67 | 67.321 | 24.113 | −37.706 | 1.00 | 51.03 | C |
| ATOM | 6764 | CD1 | LEU | D | 67 | 67.290 | 25.460 | −37.017 | 1.00 | 64.04 | C |
| ATOM | 6765 | CD2 | LEU | D | 67 | 67.356 | 22.988 | −36.698 | 1.00 | 48.12 | C |
| ATOM | 6766 | N | THR | D | 68 | 64.026 | 23.183 | −40.541 | 1.00 | 35.88 | N |
| ATOM | 6767 | CA | THR | D | 68 | 62.681 | 23.170 | −41.094 | 1.00 | 38.07 | C |
| ATOM | 6768 | C | THR | D | 68 | 62.144 | 21.741 | −41.282 | 1.00 | 39.06 | C |
| ATOM | 6769 | O | THR | D | 68 | 60.975 | 21.470 | −40.995 | 1.00 | 47.55 | O |
| ATOM | 6770 | CB | THR | D | 68 | 62.595 | 23.968 | −42.420 | 1.00 | 37.58 | C |
| ATOM | 6771 | OG1 | THR | D | 68 | 62.813 | 25.362 | −42.165 | 1.00 | 33.31 | O |
| ATOM | 6772 | CG2 | THR | D | 68 | 61.231 | 23.803 | −43.062 | 1.00 | 39.21 | C |
| ATOM | 6773 | N | ASN | D | 69 | 62.996 | 20.823 | −41.731 | 1.00 | 32.16 | N |
| ATOM | 6774 | CA | ASN | D | 69 | 62.562 | 19.448 | −42.004 | 1.00 | 35.45 | C |
| ATOM | 6775 | C | ASN | D | 69 | 62.298 | 18.584 | −40.773 | 1.00 | 32.38 | C |
| ATOM | 6776 | O | ASN | D | 69 | 61.590 | 17.583 | −40.852 | 1.00 | 29.77 | O |
| ATOM | 6777 | CB | ASN | D | 69 | 63.523 | 18.748 | −42.969 | 1.00 | 36.74 | C |
| ATOM | 6778 | CG | ASN | D | 69 | 63.335 | 19.209 | −44.401 | 1.00 | 51.61 | C |
| ATOM | 6779 | OD1 | ASN | D | 69 | 62.291 | 19.763 | −44.746 | 1.00 | 46.39 | O |
| ATOM | 6780 | ND2 | ASN | D | 69 | 64.347 | 19.000 | −45.237 | 1.00 | 58.25 | N |
| ATOM | 6781 | N | LEU | D | 70 | 62.871 | 18.973 | −39.641 | 1.00 | 35.10 | N |
| ATOM | 6782 | CA | LEU | D | 70 | 62.557 | 18.339 | −38.370 | 1.00 | 32.48 | C |
| ATOM | 6783 | C | LEU | D | 70 | 61.097 | 18.621 | −38.024 | 1.00 | 32.25 | C |
| ATOM | 6784 | O | LEU | D | 70 | 60.359 | 17.730 | −37.609 | 1.00 | 26.48 | O |
| ATOM | 6785 | CB | LEU | D | 70 | 63.475 | 18.876 | −37.272 | 1.00 | 41.98 | C |
| ATOM | 6786 | CG | LEU | D | 70 | 64.810 | 18.172 | −37.053 | 1.00 | 36.46 | C |
| ATOM | 6787 | CD1 | LEU | D | 70 | 65.617 | 18.812 | −35.904 | 1.00 | 28.53 | C |
| ATOM | 6788 | CD2 | LEU | D | 70 | 64.520 | 16.712 | −36.775 | 1.00 | 36.99 | C |
| ATOM | 6789 | N | PHE | D | 71 | 60.685 | 19.868 | −38.212 | 1.00 | 26.47 | N |
| ATOM | 6790 | CA | PHE | D | 71 | 59.292 | 20.240 | −38.023 | 1.00 | 34.44 | C |
| ATOM | 6791 | C | PHE | D | 71 | 58.377 | 19.481 | −38.985 | 1.00 | 37.54 | C |
| ATOM | 6792 | O | PHE | D | 71 | 57.293 | 19.036 | −38.601 | 1.00 | 33.37 | O |
| ATOM | 6793 | CB | PHE | D | 71 | 59.114 | 21.746 | −38.208 | 1.00 | 36.89 | C |
| ATOM | 6794 | CO | PHE | D | 71 | 59.899 | 22.570 | −37.231 | 1.00 | 38.76 | C |
| ATOM | 6795 | CD1 | PHE | D | 71 | 60.115 | 22.115 | −35.947 | 1.00 | 36.35 | C |
| ATOM | 6796 | CD2 | PHE | D | 71 | 60.402 | 23.808 | −37.590 | 1.00 | 41.40 | C |
| ATOM | 6797 | CE1 | PHE | D | 71 | 60.829 | 22.874 | −35.044 | 1.00 | 38.20 | C |
| ATOM | 6798 | CE2 | PHE | D | 71 | 61.116 | 24.568 | −36.688 | 1.00 | 37.54 | C |
| ATOM | 6799 | CZ | PHE | D | 71 | 61.330 | 24.100 | −35.417 | 1.00 | 30.84 | C |
| ATOM | 6800 | N | ILE | D | 72 | 58.821 | 19.347 | −40.233 | 1.00 | 31.96 | N |
| ATOM | 6801 | CA | ILE | D | 72 | 58.094 | 18.587 | −41.238 | 1.00 | 28.11 | C |
| ATOM | 6802 | C | ILE | D | 72 | 57.831 | 17.160 | −40.753 | 1.00 | 33.36 | C |
| ATOM | 6803 | O | ILE | D | 72 | 56.767 | 16.580 | −41.015 | 1.00 | 34.23 | O |
| ATOM | 6804 | CB | ILE | D | 72 | 58.875 | 18.539 | −42.568 | 1.00 | 33.79 | C |
| ATOM | 6805 | CG1 | ILE | D | 72 | 58.883 | 19.916 | −43.241 | 1.00 | 31.94 | C |
| ATOM | 6806 | CG2 | ILE | D | 72 | 58.299 | 17.479 | −43.508 | 1.00 | 33.20 | C |
| ATOM | 6807 | CD1 | ILE | D | 72 | 57.529 | 20.511 | −43.466 | 1.00 | 24.25 | C |
| ATOM | 6808 | N | THR | D | 73 | 58.807 | 16.607 | −40.039 | 1.00 | 30.89 | N |
| ATOM | 6809 | CA | THR | D | 73 | 58.735 | 15.244 | −39.519 | 1.00 | 33.76 | C |
| ATOM | 6810 | C | THR | D | 73 | 57.726 | 15.123 | −38.385 | 1.00 | 35.41 | C |
| ATOM | 6811 | O | THR | D | 73 | 57.122 | 14.070 | −38.182 | 1.00 | 37.41 | O |
| ATOM | 6812 | CB | THR | D | 73 | 60.115 | 14.781 | −38.995 | 1.00 | 26.78 | C |
| ATOM | 6813 | OG1 | THR | D | 73 | 61.095 | 14.937 | −40.027 | 1.00 | 22.82 | O |
| ATOM | 6814 | CG2 | THR | D | 73 | 60.059 | 13.325 | −38.551 | 1.00 | 16.71 | C |
| ATOM | 6815 | N | SER | D | 74 | 57.580 | 16.201 | −37.624 | 1.00 | 31.99 | N |
| ATOM | 6816 | CA | SER | D | 74 | 56.601 | 16.244 | −36.560 | 1.00 | 35.78 | C |
| ATOM | 6817 | C | SER | D | 74 | 55.240 | 16.219 | −37.247 | 1.00 | 36.44 | C |
| ATOM | 6818 | O | SER | D | 74 | 54.337 | 15.466 | −36.863 | 1.00 | 32.34 | O |
| ATOM | 6819 | CB | SER | D | 74 | 56.782 | 17.527 | −35.734 | 1.00 | 35.41 | C |
| ATOM | 6820 | OG | SER | D | 74 | 55.823 | 17.636 | −34.694 | 1.00 | 41.53 | O |
| ATOM | 6821 | N | LEU | D | 75 | 55.133 | 17.040 | −38.288 | 1.00 | 29.06 | N |
| ATOM | 6822 | CA | LEU | D | 75 | 53.929 | 17.179 | −39.083 | 1.00 | 23.55 | C |
| ATOM | 6823 | C | LEU | D | 75 | 53.495 | 15.858 | −39.702 | 1.00 | 28.81 | C |
| ATOM | 6824 | O | LEU | D | 75 | 52.300 | 15.584 | −39.820 | 1.00 | 31.50 | O |
| ATOM | 6825 | CB | LEU | D | 75 | 54.169 | 18.203 | −40.181 | 1.00 | 24.08 | C |
| ATOM | 6826 | CG | LEU | D | 75 | 52.915 | 18.914 | −40.667 | 1.00 | 26.85 | C |
| ATOM | 6827 | CD1 | LEU | D | 75 | 51.889 | 18.976 | −39.545 | 1.00 | 22.32 | C |
| ATOM | 6828 | CD2 | LEU | D | 75 | 53.284 | 20.301 | −41.177 | 1.00 | 22.76 | C |
| ATOM | 6829 | N | ALA | D | 76 | 54.470 | 15.042 | −40.092 | 1.00 | 25.99 | N |
| ATOM | 6830 | CA | ALA | D | 76 | 54.209 | 13.740 | −40.690 | 1.00 | 25.57 | C |
| ATOM | 6831 | C | ALA | D | 76 | 53.797 | 12.662 | −39.676 | 1.00 | 27.01 | C |
| ATOM | 6832 | O | ALA | D | 76 | 53.124 | 11.694 | −40.024 | 1.00 | 30.47 | O |
| ATOM | 6833 | CB | ALA | D | 76 | 55.415 | 13.287 | −41.486 | 1.00 | 22.65 | C |
| ATOM | 6834 | N | CYS | D | 77 | 54.209 | 12.818 | −38.425 | 1.00 | 28.50 | N |
| ATOM | 6835 | CA | CYS | D | 77 | 53.779 | 11.893 | −37.385 | 1.00 | 33.90 | C |
| ATOM | 6836 | C | CYS | D | 77 | 52.332 | 12.162 | −36.983 | 1.00 | 31.42 | C |
| ATOM | 6837 | O | CYS | D | 77 | 51.557 | 11.228 | −36.794 | 1.00 | 27.61 | O |
| ATOM | 6838 | CB | CYS | D | 77 | 54.710 | 11.968 | −36.181 | 1.00 | 31.40 | C |
| ATOM | 6839 | SG | CYS | D | 77 | 56.357 | 11.314 | −36.539 | 1.00 | 41.80 | S |
| ATOM | 6840 | N | ALA | D | 78 | 51.974 | 13.439 | −36.865 | 1.00 | 30.36 | N |
| ATOM | 6841 | CA | ALA | D | 78 | 50.578 | 13.812 | −36.695 | 1.00 | 30.55 | C |
| ATOM | 6842 | C | ALA | D | 78 | 49.774 | 13.058 | −37.741 | 1.00 | 31.48 | C |
| ATOM | 6843 | O | ALA | D | 78 | 48.719 | 12.502 | −37.448 | 1.00 | 28.66 | O |
| ATOM | 6844 | CB | ALA | D | 78 | 50.392 | 15.308 | −36.870 | 1.00 | 20.44 | C |
| ATOM | 6845 | N | ASP | D | 79 | 50.311 | 13.023 | −38.959 | 1.00 | 31.82 | N |
| ATOM | 6846 | CA | ASP | D | 79 | 49.626 | 12.416 | −40.095 | 1.00 | 26.35 | C |
| ATOM | 6847 | C | ASP | D | 79 | 49.643 | 10.899 | −40.052 | 1.00 | 25.56 | C |
| ATOM | 6848 | O | ASP | D | 79 | 48.657 | 10.250 | −40.380 | 1.00 | 25.49 | O |
| ATOM | 6849 | CB | ASP | D | 79 | 50.201 | 12.930 | −41.407 | 1.00 | 22.71 | C |
| ATOM | 6850 | CG | ASP | D | 79 | 49.593 | 14.261 | −41.826 | 1.00 | 34.62 | C |
| ATOM | 6851 | OD1 | ASP | D | 79 | 48.789 | 14.828 | −41.052 | 1.00 | 37.53 | O |
| ATOM | 6852 | OD2 | ASP | D | 79 | 49.916 | 14.745 | −42.934 | 1.00 | 54.22 | O |
| ATOM | 6853 | N | LEU | D | 80 | 50.760 | 10.332 | −39.622 | 1.00 | 31.51 | N |
| ATOM | 6854 | CA | LEU | D | 80 | 50.832 | 8.896 | −39.419 | 1.00 | 33.76 | C |
| ATOM | 6855 | C | LEU | D | 80 | 49.759 | 8.431 | −38.419 | 1.00 | 38.62 | C |
| ATOM | 6856 | O | LEU | D | 80 | 49.118 | 7.390 | −38.599 | 1.00 | 34.43 | O |
| ATOM | 6857 | CB | LEU | D | 80 | 52.234 | 8.502 | −38.948 | 1.00 | 38.39 | C |
| ATOM | 6858 | CG | LEU | D | 80 | 52.699 | 7.136 | −39.446 | 1.00 | 38.27 | C |
| ATOM | 6859 | CD1 | LEU | D | 80 | 52.291 | 6.964 | −40.891 | 1.00 | 36.50 | C |
| ATOM | 6860 | CD2 | LEU | D | 80 | 54.199 | 6.997 | −39.295 | 1.00 | 35.72 | C |
| ATOM | 6861 | N | VAL | D | 81 | 49.550 | 9.206 | −37.365 | 1.00 | 33.39 | N |
| ATOM | 6862 | CA | VAL | D | 81 | 48.569 | 8.814 | −36.369 | 1.00 | 37.23 | C |
| ATOM | 6863 | C | VAL | D | 81 | 47.149 | 8.899 | −36.936 | 1.00 | 32.14 | C |
| ATOM | 6864 | O | VAL | D | 81 | 46.354 | 7.976 | −36.770 | 1.00 | 37.01 | O |
| ATOM | 6865 | CB | VAL | D | 81 | 48.741 | 9.610 | −35.051 | 1.00 | 43.49 | C |
| ATOM | 6866 | CG1 | VAL | D | 81 | 47.574 | 9.356 | −34.089 | 1.00 | 24.72 | C |
| ATOM | 6867 | CG2 | VAL | D | 81 | 50.070 | 9.234 | −34.404 | 1.00 | 25.07 | C |
| ATOM | 6868 | N | VAL | D | 82 | 46.839 | 9.994 | −37.620 | 1.00 | 30.23 | N |
| ATOM | 6869 | CA | VAL | D | 82 | 45.560 | 10.124 | −38.320 | 1.00 | 35.59 | C |
| ATOM | 6870 | C | VAL | D | 82 | 45.280 | 8.942 | −39.255 | 1.00 | 33.53 | C |
| ATOM | 6871 | O | VAL | D | 82 | 44.138 | 8.505 | −39.401 | 1.00 | 29.91 | O |
| ATOM | 6872 | CB | VAL | D | 82 | 45.518 | 11.406 | −39.168 | 1.00 | 29.15 | C |
| ATOM | 6873 | CG1 | VAL | D | 82 | 44.257 | 11.434 | −40.031 | 1.00 | 25.07 | C |
| ATOM | 6874 | CG2 | VAL | D | 82 | 45.615 | 12.631 | −38.272 | 1.00 | 26.31 | C |
| ATOM | 6875 | N | GLY | D | 83 | 46.334 | 8.431 | −39.881 | 1.00 | 31.84 | N |
| ATOM | 6876 | CA | GLY | D | 83 | 46.208 | 7.364 | −40.853 | 1.00 | 37.73 | C |
| ATOM | 6877 | C | GLY | D | 83 | 46.185 | 5.950 | −40.304 | 1.00 | 32.19 | C |
| ATOM | 6878 | O | GLY | D | 83 | 45.788 | 5.023 | −40.996 | 1.00 | 37.70 | O |
| ATOM | 6879 | N | LEU | D | 84 | 46.614 | 5.764 | −39.067 | 1.00 | 35.57 | N |
| ATOM | 6880 | CA | LEU | D | 84 | 46.685 | 4.416 | −38.520 | 1.00 | 42.13 | C |
| ATOM | 6881 | C | LEU | D | 84 | 45.612 | 4.149 | −37.462 | 1.00 | 41.43 | C |
| ATOM | 6882 | O | LEU | D | 84 | 45.114 | 3.029 | −37.340 | 1.00 | 45.07 | O |
| ATOM | 6883 | CB | LEU | D | 84 | 48.083 | 4.145 | −37.955 | 1.00 | 39.34 | C |
| ATOM | 6884 | CG | LEU | D | 84 | 49.187 | 4.014 | −39.002 | 1.00 | 44.93 | C |
| ATOM | 6885 | CD1 | LEU | D | 84 | 50.565 | 3.964 | −38.354 | 1.00 | 33.60 | C |
| ATOM | 6886 | CD2 | LEU | D | 84 | 48.940 | 2.776 | −39.842 | 1.00 | 44.70 | C |
| ATOM | 6887 | N | LEU | D | 85 | 45.253 | 5.177 | −36.703 | 1.00 | 34.03 | N |
| ATOM | 6888 | CA | LEU | D | 85 | 44.293 | 5.004 | −35.624 | 1.00 | 39.34 | C |
| ATOM | 6889 | C | LEU | D | 85 | 42.968 | 5.712 | −35.889 | 1.00 | 36.37 | C |
| ATOM | 6890 | O | LEU | D | 85 | 41.908 | 5.102 | −35.791 | 1.00 | 46.03 | O |
| ATOM | 6891 | CB | LEU | D | 85 | 44.910 | 5.459 | −34.303 | 1.00 | 40.89 | C |
| ATOM | 6892 | CG | LEU | D | 85 | 46.274 | 4.800 | −34.081 | 1.00 | 41.00 | C |
| ATOM | 6893 | CD1 | LEU | D | 85 | 46.977 | 5.398 | −32.897 | 1.00 | 27.73 | C |
| ATOM | 6894 | CD2 | LEU | D | 85 | 46.128 | 3.292 | −33.914 | 1.00 | 36.09 | C |
| ATOM | 6895 | N | VAL | D | 86 | 43.030 | 6.987 | −36.253 | 1.00 | 32.34 | N |
| ATOM | 6896 | CA | VAL | D | 86 | 41.825 | 7.789 | −36.449 | 1.00 | 28.11 | C |
| ATOM | 6897 | C | VAL | D | 86 | 40.971 | 7.325 | −37.640 | 1.00 | 26.83 | C |
| ATOM | 6898 | O | VAL | D | 86 | 39.848 | 6.861 | −37.482 | 1.00 | 24.50 | O |
| ATOM | 6899 | CB | VAL | D | 86 | 42.186 | 9.280 | −36.632 | 1.00 | 22.85 | C |
| ATOM | 6900 | CG1 | VAL | D | 86 | 40.940 | 10.108 | −36.883 | 1.00 | 21.57 | C |
| ATOM | 6901 | CG2 | VAL | D | 86 | 42.944 | 9.794 | −35.436 | 1.00 | 19.43 | C |
| ATOM | 6902 | N | VAL | D | 87 | 41.514 | 7.458 | −38.842 | 1.00 | 37.84 | N |
| ATOM | 6903 | CA | VAL | D | 87 | 40.756 | 7.163 | −40.059 | 1.00 | 33.94 | C |
| ATOM | 6904 | C | VAL | D | 87 | 40.257 | 5.711 | −40.168 | 1.00 | 29.65 | C |
| ATOM | 6905 | O | VAL | D | 87 | 39.112 | 5.480 | −40.539 | 1.00 | 21.68 | O |
| ATOM | 6906 | CB | VAL | D | 87 | 41.543 | 7.573 | −41.324 | 1.00 | 29.08 | C |
| ATOM | 6907 | CG1 | VAL | D | 87 | 41.107 | 6.750 | −42.501 | 1.00 | 32.70 | C |
| ATOM | 6908 | CG2 | VAL | D | 87 | 41.338 | 9.049 | −41.612 | 1.00 | 33.35 | C |
| ATOM | 6909 | N | PRO | D | 88 | 41.119 | 4.730 | −39.851 | 1.00 | 31.52 | N |
| ATOM | 6910 | CA | PRO | D | 88 | 40.611 | 3.362 | −39.959 | 1.00 | 28.00 | C |
| ATOM | 6911 | C | PRO | D | 88 | 39.378 | 3.118 | −39.097 | 1.00 | 28.75 | C |
| ATOM | 6912 | O | PRO | D | 88 | 38.404 | 2.562 | −39.592 | 1.00 | 35.20 | O |
| ATOM | 6913 | CB | PRO | D | 88 | 41.797 | 2.508 | −39.512 | 1.00 | 24.80 | C |
| ATOM | 6914 | CG | PRO | D | 88 | 42.978 | 3.320 | −39.880 | 1.00 | 26.83 | C |
| ATOM | 6915 | CD | PRO | D | 88 | 42.582 | 4.760 | −39.670 | 1.00 | 31.53 | C |
| ATOM | 6916 | N | PHE | D | 89 | 39.398 | 3.532 | −37.838 | 1.00 | 35.40 | N |
| ATOM | 6917 | CA | PHE | D | 89 | 38.225 | 3.329 | −36.993 | 1.00 | 29.86 | C |
| ATOM | 6918 | C | PHE | D | 89 | 37.039 | 4.189 | −37.447 | 1.00 | 31.05 | C |
| ATOM | 6919 | O | PHE | D | 89 | 35.896 | 3.731 | −37.447 | 1.00 | 25.12 | O |
| ATOM | 6920 | CB | PHE | D | 89 | 38.581 | 3.522 | −35.521 | 1.00 | 25.15 | C |
| ATOM | 6921 | CG | PHE | D | 89 | 39.414 | 2.406 | −34.967 | 1.00 | 31.65 | C |
| ATOM | 6922 | CD1 | PHE | D | 89 | 38.815 | 1.305 | −34.377 | 1.00 | 28.63 | C |
| ATOM | 6923 | CD2 | PHE | D | 89 | 40.798 | 2.435 | −35.077 | 1.00 | 28.72 | C |
| ATOM | 6924 | CE1 | PHE | D | 89 | 39.580 | 0.261 | −33.875 | 1.00 | 31.49 | C |
| ATOM | 6925 | CE2 | PHE | D | 89 | 41.573 | 1.397 | −34.582 | 1.00 | 25.31 | C |
| ATOM | 6926 | CZ | PHE | D | 89 | 40.961 | 0.307 | −33.980 | 1.00 | 29.91 | C |
| ATOM | 6927 | N | GLY | D | 90 | 37.323 | 5.418 | −37.871 | 1.00 | 27.71 | N |
| ATOM | 6928 | CA | GLY | D | 90 | 36.300 | 6.292 | −38.410 | 1.00 | 28.44 | C |
| ATOM | 6929 | C | GLY | D | 90 | 35.602 | 5.696 | −39.626 | 1.00 | 42.35 | C |
| ATOM | 6930 | O | GLY | D | 90 | 34.401 | 5.905 | −39.838 | 1.00 | 36.51 | O |
| ATOM | 6931 | N | ALA | D | 91 | 36.364 | 4.960 | −40.432 | 1.00 | 38.65 | N |
| ATOM | 6932 | CA | ALA | D | 91 | 35.817 | 4.243 | −41.580 | 1.00 | 40.10 | C |
| ATOM | 6933 | C | ALA | D | 91 | 34.797 | 3.162 | −41.178 | 1.00 | 39.33 | C |
| ATOM | 6934 | O | ALA | D | 91 | 33.736 | 3.057 | −41.783 | 1.00 | 38.31 | O |
| ATOM | 6935 | CB | ALA | D | 91 | 36.940 | 3.634 | −42.411 | 1.00 | 26.37 | C |
| ATOM | 6936 | N | THR | D | 92 | 35.119 | 2.359 | −40.167 | 1.00 | 30.04 | N |
| ATOM | 6937 | CA | THR | D | 92 | 34.186 | 1.339 | −39.700 | 1.00 | 35.03 | C |
| ATOM | 6938 | C | THR | D | 92 | 32.864 | 1.981 | −39.275 | 1.00 | 41.42 | C |
| ATOM | 6939 | O | THR | D | 92 | 31.791 | 1.446 | −39.534 | 1.00 | 45.42 | O |
| ATOM | 6940 | CB | THR | D | 92 | 34.761 | 0.492 | −38.532 | 1.00 | 31.39 | C |
| ATOM | 6941 | OG1 | THR | D | 92 | 35.047 | 1.337 | −37.410 | 1.00 | 31.55 | O |
| ATOM | 6942 | CG2 | THR | D | 92 | 36.039 | −0.227 | −38.957 | 1.00 | 35.33 | C |
| ATOM | 6943 | N | LEU | D | 93 | 32.951 | 3.145 | −38.642 | 1.00 | 37.78 | N |
| ATOM | 6944 | CA | LEU | D | 93 | 31.775 | 3.843 | −38.154 | 1.00 | 38.85 | C |
| ATOM | 6945 | C | LEU | D | 93 | 30.891 | 4.369 | −39.289 | 1.00 | 41.77 | C |
| ATOM | 6946 | O | LEU | D | 93 | 29.707 | 4.041 | −39.387 | 1.00 | 41.55 | O |
| ATOM | 6947 | CB | LEU | D | 93 | 32.200 | 4.993 | −37.240 | 1.00 | 41.11 | C |
| ATOM | 6948 | CG | LEU | D | 93 | 31.094 | 5.876 | −36.652 | 1.00 | 40.17 | C |
| ATOM | 6949 | CD1 | LEU | D | 93 | 30.158 | 5.071 | −35.753 | 1.00 | 32.64 | C |
| ATOM | 6950 | CD2 | LEU | D | 93 | 31.721 | 7.020 | −35.888 | 1.00 | 31.93 | C |
| ATOM | 6951 | N | VAL | D | 94 | 31.462 | 5.201 | −40.143 | 1.00 | 43.22 | N |
| ATOM | 6952 | CA | VAL | D | 94 | 30.691 | 5.767 | −41.242 | 1.00 | 53.36 | C |
| ATOM | 6953 | C | VAL | D | 94 | 30.062 | 4.657 | −42.087 | 1.00 | 49.22 | C |
| ATOM | 6954 | O | VAL | D | 94 | 28.896 | 4.737 | −42.462 | 1.00 | 48.15 | O |
| ATOM | 6955 | CB | VAL | D | 94 | 31.543 | 6.706 | −42.125 | 1.00 | 48.09 | C |
| ATOM | 6956 | CG1 | VAL | D | 94 | 30.674 | 7.390 | −43.140 | 1.00 | 45.33 | C |
| ATOM | 6957 | CG2 | VAL | D | 94 | 32.244 | 7.753 | −41.272 | 1.00 | 47.48 | C |
| ATOM | 6958 | N | VAL | D | 95 | 30.828 | 3.615 | −42.374 | 1.00 | 44.62 | N |
| ATOM | 6959 | CA | VAL | D | 95 | 30.298 | 2.495 | −43.145 | 1.00 | 53.38 | C |
| ATOM | 6960 | C | VAL | D | 95 | 29.296 | 1.672 | −42.344 | 1.00 | 52.07 | C |
| ATOM | 6961 | O | VAL | D | 95 | 28.097 | 1.732 | −42.599 | 1.00 | 56.06 | O |
| ATOM | 6962 | CB | VAL | D | 95 | 31.419 | 1.582 | −43.702 | 1.00 | 58.28 | C |
| ATOM | 6963 | CG1 | VAL | D | 95 | 30.851 | 0.245 | −44.167 | 1.00 | 57.40 | C |
| ATOM | 6964 | CG2 | VAL | D | 95 | 32.142 | 2.275 | −44.848 | 1.00 | 53.27 | C |
| ATOM | 6965 | N | ARG | D | 96 | 29.800 | 0.916 | −41.376 | 1.00 | 49.62 | N |
| ATOM | 6966 | CA | ARG | D | 96 | 28.966 | 0.074 | −40.529 | 1.00 | 56.75 | C |
| ATOM | 6967 | C | ARG | D | 96 | 27.721 | 0.801 | −39.977 | 1.00 | 54.46 | C |
| ATOM | 6968 | O | ARG | D | 96 | 26.693 | 0.175 | −39.702 | 1.00 | 52.19 | O |
| ATOM | 6969 | CB | ARG | D | 96 | 29.815 | −0.474 | −39.387 | 1.00 | 52.90 | C |
| ATOM | 6970 | CG | ARG | D | 96 | 29.068 | −1.352 | −38.426 | 1.00 | 80.81 | C |
| ATOM | 6971 | CD | ARG | D | 96 | 28.655 | −2.626 | −39.111 | 1.00 | 96.40 | C |
| ATOM | 6972 | NE | ARG | D | 96 | 27.368 | −3.086 | −38.611 | 1.00 | 104.56 | N |
| ATOM | 6973 | CZ | ARG | D | 96 | 26.588 | −3.933 | −39.266 | 1.00 | 114.67 | C |
| ATOM | 6974 | NH1 | ARG | D | 96 | 26.983 | −4.411 | −40.442 | 1.00 | 104.14 | N |
| ATOM | 6975 | NH2 | ARG | D | 96 | 25.427 | −4.308 | −38.743 | 1.00 | 115.17 | N |
| ATOM | 6976 | N | GLY | D | 97 | 27.825 | 2.120 | −39.829 | 1.00 | 47.44 | N |
| ATOM | 6977 | CA | GLY | D | 97 | 26.760 | 2.944 | −39.281 | 1.00 | 35.72 | C |
| ATOM | 6978 | C | GLY | D | 97 | 26.660 | 2.893 | −37.760 | 1.00 | 46.17 | C |
| ATOM | 6979 | O | GLY | D | 97 | 25.685 | 3.349 | −37.180 | 1.00 | 37.40 | O |
| ATOM | 6980 | N | THR | D | 98 | 27.670 | 2.335 | −37.105 | 1.00 | 49.27 | N |
| ATOM | 6981 | CA | THR | D | 98 | 27.623 | 2.158 | −35.658 | 1.00 | 47.11 | C |
| ATOM | 6982 | C | THR | D | 98 | 29.022 | 2.056 | −35.079 | 1.00 | 44.03 | C |
| ATOM | 6983 | O | THR | D | 98 | 29.947 | 1.632 | −35.777 | 1.00 | 42.85 | O |
| ATOM | 6984 | CB | THR | D | 98 | 26.797 | 0.911 | −35.274 | 1.00 | 42.09 | C |
| ATOM | 6985 | OG1 | THR | D | 98 | 25.495 | 1.324 | −34.854 | 1.00 | 53.91 | O |
| ATOM | 6986 | CG2 | THR | D | 98 | 27.448 | 0.155 | −34.135 | 1.00 | 46.93 | C |
| ATOM | 6987 | N | TRP | D | 99 | 29.179 | 2.464 | −33.818 | 1.00 | 33.43 | N |
| ATOM | 6988 | CA | TRP | D | 99 | 30.462 | 2.314 | −33.140 | 1.00 | 33.99 | C |
| ATOM | 6989 | C | TRP | D | 99 | 30.612 | 0.911 | −32.586 | 1.00 | 29.88 | C |
| ATOM | 6990 | O | TRP | D | 99 | 29.775 | 0.454 | −31.820 | 1.00 | 38.52 | O |
| ATOM | 6991 | CB | TRP | D | 99 | 30.665 | 3.343 | −32.030 | 1.00 | 29.12 | C |
| ATOM | 6992 | CG | TRP | D | 99 | 32.025 | 3.223 | −31.464 | 1.00 | 30.04 | C |
| ATOM | 6993 | CD1 | TRP | D | 99 | 32.380 | 2.612 | −30.300 | 1.00 | 34.38 | C |
| ATOM | 6994 | CD2 | TRP | D | 99 | 33.240 | 3.675 | −32.067 | 1.00 | 37.82 | C |
| ATOM | 6995 | NE1 | TRP | D | 99 | 33.738 | 2.682 | −30.120 | 1.00 | 29.98 | N |
| ATOM | 6996 | CE2 | TRP | D | 99 | 34.294 | 3.330 | −31.194 | 1.00 | 41.85 | C |
| ATOM | 6997 | CE3 | TRP | D | 99 | 33.543 | 4.349 | −33.259 | 1.00 | 31.66 | C |
| ATOM | 6998 | CZ2 | TRP | D | 99 | 35.633 | 3.635 | −31.475 | 1.00 | 31.06 | C |
| ATOM | 6999 | CZ3 | TRP | D | 99 | 34.872 | 4.661 | −33.526 | 1.00 | 27.51 | C |
| ATOM | 7000 | CH2 | TRP | D | 99 | 35.895 | 4.300 | −32.639 | 1.00 | 22.61 | C |
| ATOM | 7001 | N | LEU | D | 100 | 31.696 | 0.242 | −32.966 | 1.00 | 32.61 | N |
| ATOM | 7002 | CA | LEU | D | 100 | 31.850 | −1.190 | −32.730 | 1.00 | 31.51 | C |
| ATOM | 7003 | C | LEU | D | 100 | 32.892 | −1.516 | −31.676 | 1.00 | 31.85 | C |
| ATOM | 7004 | O | LEU | D | 100 | 33.167 | −2.687 | −31.403 | 1.00 | 35.77 | O |
| ATOM | 7005 | CB | LEU | D | 100 | 32.244 | −1.897 | −34.036 | 1.00 | 36.48 | C |
| ATOM | 7006 | CG | LEU | D | 100 | 31.221 | −1.918 | −35.174 | 1.00 | 47.33 | C |
| ATOM | 7007 | CD1 | LEU | D | 100 | 31.820 | −2.526 | −36.429 | 1.00 | 41.55 | C |
| ATOM | 7008 | CD2 | LEU | D | 100 | 29.980 | −2.680 | −34.750 | 1.00 | 29.84 | C |
| ATOM | 7009 | N | TRP | D | 101 | 33.482 | −0.494 | −31.081 | 1.00 | 26.11 | N |
| ATOM | 7010 | CA | TRP | D | 101 | 34.654 | −0.742 | −30.263 | 1.00 | 33.17 | C |
| ATOM | 7011 | C | TRP | D | 101 | 34.505 | −0.484 | −28.752 | 1.00 | 31.78 | C |
| ATOM | 7012 | O | TRP | D | 101 | 35.458 | −0.642 | −28.001 | 1.00 | 35.01 | O |
| ATOM | 7013 | CB | TRP | D | 101 | 35.837 | 0.023 | −30.860 | 1.00 | 34.73 | C |
| ATOM | 7014 | CG | TRP | D | 101 | 35.982 | −0.256 | −32.319 | 1.00 | 23.39 | C |
| ATOM | 7015 | CD1 | TRP | D | 101 | 35.435 | 0.451 | −33.344 | 1.00 | 32.33 | C |
| ATOM | 7016 | CD2 | TRP | D | 101 | 36.705 | −1.330 | −32.914 | 1.00 | 25.24 | C |
| ATOM | 7017 | NE1 | TRP | D | 101 | 35.778 | −0.112 | −34.554 | 1.00 | 27.57 | N |
| ATOM | 7018 | CE2 | TRP | D | 101 | 36.556 | −1.208 | −34.317 | 1.00 | 26.96 | C |
| ATOM | 7019 | CE3 | TRP | D | 101 | 37.478 | −2.374 | −32.407 | 1.00 | 26.35 | C |
| ATOM | 7020 | CZ2 | TRP | D | 101 | 37.143 | −2.092 | −35.208 | 1.00 | 27.47 | C |
| ATOM | 7021 | CZ3 | TRP | D | 101 | 38.064 | −3.253 | −33.293 | 1.00 | 32.72 | C |
| ATOM | 7022 | CH2 | TRP | D | 101 | 37.892 | −3.110 | −34.679 | 1.00 | 38.55 | C |
| ATOM | 7023 | N | GLY | D | 102 | 33.315 | −0.104 | −28.306 | 1.00 | 39.06 | N |
| ATOM | 7024 | CA | GLY | D | 102 | 33.109 | 0.163 | −26.894 | 1.00 | 44.18 | C |
| ATOM | 7025 | C | GLY | D | 102 | 33.325 | 1.626 | −26.568 | 1.00 | 44.57 | C |
| ATOM | 7026 | O | GLY | D | 102 | 34.031 | 2.323 | −27.295 | 1.00 | 44.14 | O |
| ATOM | 7027 | N | SER | D | 103 | 32.730 | 2.092 | −25.471 | 1.00 | 53.52 | N |
| ATOM | 7028 | CA | SER | D | 103 | 32.701 | 3.527 | −25.175 | 1.00 | 50.60 | C |
| ATOM | 7029 | C | SER | D | 103 | 34.065 | 4.142 | −24.844 | 1.00 | 37.48 | C |
| ATOM | 7030 | O | SER | D | 103 | 34.329 | 5.282 | −25.215 | 1.00 | 44.41 | O |
| ATOM | 7031 | CB | SER | D | 103 | 31.671 | 3.847 | −24.091 | 1.00 | 36.24 | C |
| ATOM | 7032 | OG | SER | D | 103 | 31.813 | 2.978 | −22.990 | 1.00 | 47.97 | O |
| ATOM | 7033 | N | PHE | D | 104 | 34.932 | 3.397 | −24.167 | 1.00 | 32.98 | N |
| ATOM | 7034 | CA | PHE | D | 104 | 36.278 | 3.896 | −23.895 | 1.00 | 39.66 | C |
| ATOM | 7035 | C | PHE | D | 104 | 37.129 | 4.162 | −25.148 | 1.00 | 37.87 | C |
| ATOM | 7036 | O | PHE | D | 104 | 37.765 | 5.209 | −25.271 | 1.00 | 26.43 | O |
| ATOM | 7037 | CB | PHE | D | 104 | 37.060 | 2.953 | −22.991 | 1.00 | 32.70 | C |
| ATOM | 7038 | CG | PHE | D | 104 | 38.482 | 3.367 | −22.825 | 1.00 | 34.01 | C |
| ATOM | 7039 | CD1 | PHE | D | 104 | 38.826 | 4.359 | −21.913 | 1.00 | 35.26 | C |
| ATOM | 7040 | CD2 | PHE | D | 104 | 39.473 | 2.817 | −23.624 | 1.00 | 39.77 | C |
| ATOM | 7041 | CE1 | PHE | D | 104 | 40.143 | 4.768 | −21.774 | 1.00 | 41.98 | C |
| ATOM | 7042 | CE2 | PHE | D | 104 | 40.796 | 3.214 | −23.487 | 1.00 | 39.06 | C |
| ATOM | 7043 | CZ | PHE | D | 104 | 41.132 | 4.192 | −22.564 | 1.00 | 36.64 | C |
| ATOM | 7044 | N | LEU | D | 105 | 37.167 | 3.191 | −26.052 | 1.00 | 31.65 | N |
| ATOM | 7045 | CA | LEU | D | 105 | 37.885 | 3.366 | −27.302 | 1.00 | 34.85 | C |
| ATOM | 7046 | C | LEU | D | 105 | 37.282 | 4.512 | −28.122 | 1.00 | 33.62 | C |
| ATOM | 7047 | O | LEU | D | 105 | 37.993 | 5.218 | −28.844 | 1.00 | 21.47 | O |
| ATOM | 7048 | CB | LEU | D | 105 | 37.927 | 2.058 | −28.097 | 1.00 | 31.29 | C |
| ATOM | 7049 | CG | LEU | D | 105 | 39.003 | 1.096 | −27.583 | 1.00 | 32.71 | C |
| ATOM | 7050 | CD1 | LEU | D | 105 | 39.010 | −0.219 | −28.373 | 1.00 | 23.98 | C |
| ATOM | 7051 | CD2 | LEU | D | 105 | 40.369 | 1.779 | −27.621 | 1.00 | 26.77 | C |
| ATOM | 7052 | N | CYS | D | 106 | 35.974 | 4.712 | −27.985 | 1.00 | 28.78 | N |
| ATOM | 7053 | CA | CYS | D | 106 | 35.326 | 5.854 | −28.607 | 1.00 | 25.43 | C |
| ATOM | 7054 | C | CYS | D | 106 | 35.891 | 7.159 | −28.055 | 1.00 | 29.61 | C |
| ATOM | 7055 | O | CYS | D | 106 | 36.194 | 8.090 | −28.806 | 1.00 | 25.98 | O |
| ATOM | 7056 | CB | CYS | D | 106 | 33.814 | 5.805 | −28.408 | 1.00 | 31.59 | C |
| ATOM | 7057 | SG | CYS | D | 106 | 32.967 | 7.370 | −28.767 | 1.00 | 35.64 | S |
| ATOM | 7058 | N | GLU | D | 107 | 36.047 | 7.233 | −26.741 | 1.00 | 27.17 | N |
| ATOM | 7059 | CA | GLU | D | 107 | 36.561 | 8.458 | −26.147 | 1.00 | 25.63 | C |
| ATOM | 7060 | C | GLU | D | 107 | 38.070 | 8.630 | −26.350 | 1.00 | 25.32 | C |
| ATOM | 7061 | O | GLU | D | 107 | 38.552 | 9.750 | −26.520 | 1.00 | 22.17 | O |
| ATOM | 7062 | CB | GLU | D | 107 | 36.159 | 8.559 | −24.681 | 1.00 | 30.41 | C |
| ATOM | 7063 | CG | GLU | D | 107 | 34.670 | 8.745 | −24.504 | 1.00 | 25.62 | C |
| ATOM | 7064 | CD | GLU | D | 107 | 34.210 | 8.501 | −23.083 | 1.00 | 37.52 | C |
| ATOM | 7065 | OE1 | GLU | D | 107 | 34.938 | 8.879 | −22.137 | 1.00 | 45.13 | O |
| ATOM | 7066 | OE2 | GLU | D | 107 | 33.110 | 7.936 | −22.913 | 1.00 | 50.79 | O |
| ATOM | 7067 | N | LEU | D | 108 | 38.810 | 7.527 | −26.362 | 1.00 | 20.38 | N |
| ATOM | 7068 | CA | LEU | D | 108 | 40.228 | 7.584 | −26.717 | 1.00 | 27.40 | C |
| ATOM | 7069 | C | LEU | D | 108 | 40.449 | 8.014 | −28.178 | 1.00 | 32.97 | C |
| ATOM | 7070 | O | LEU | D | 108 | 41.340 | 8.813 | −28.478 | 1.00 | 25.92 | O |
| ATOM | 7071 | CB | LEU | D | 108 | 40.908 | 6.244 | −26.462 | 1.00 | 25.29 | C |
| ATOM | 7072 | CG | LEU | D | 108 | 42.382 | 6.240 | −26.859 | 1.00 | 23.27 | C |
| ATOM | 7073 | CD1 | LEU | D | 108 | 43.104 | 7.426 | −26.225 | 1.00 | 23.41 | C |
| ATOM | 7074 | CD2 | LEU | D | 108 | 43.051 | 4.919 | −26.488 | 1.00 | 20.13 | C |
| ATOM | 7075 | N | TRP | D | 109 | 39.616 | 7.470 | −29.064 | 1.00 | 30.02 | N |
| ATOM | 7076 | CA | TRP | D | 109 | 39.598 | 7.781 | −30.489 | 1.00 | 20.96 | C |
| ATOM | 7077 | C | TRP | D | 109 | 39.370 | 9.259 | −30.766 | 1.00 | 26.35 | C |
| ATOM | 7078 | O | TRP | D | 109 | 40.109 | 9.884 | −31.533 | 1.00 | 29.28 | O |
| ATOM | 7079 | CB | TRP | D | 109 | 38.481 | 6.974 | −31.163 | 1.00 | 21.29 | C |
| ATOM | 7080 | CG | TRP | D | 109 | 38.306 | 7.272 | −32.626 | 1.00 | 27.20 | C |
| ATOM | 7081 | CD1 | TRP | D | 109 | 39.224 | 7.063 | −33.619 | 1.00 | 21.75 | C |
| ATOM | 7082 | CD2 | TRP | D | 109 | 37.139 | 7.807 | −33.268 | 1.00 | 28.71 | C |
| ATOM | 7083 | NE1 | TRP | D | 109 | 38.715 | 7.455 | −34.824 | 1.00 | 22.69 | N |
| ATOM | 7084 | CE2 | TRP | D | 109 | 37.437 | 7.912 | −34.646 | 1.00 | 26.89 | C |
| ATOM | 7085 | CE3 | TRP | D | 109 | 35.880 | 8.213 | −32.818 | 1.00 | 24.04 | C |
| ATOM | 7086 | CZ2 | TRP | D | 109 | 36.517 | 8.407 | −35.580 | 1.00 | 29.22 | C |
| ATOM | 7087 | CZ3 | TRP | D | 109 | 34.967 | 8.715 | −33.744 | 1.00 | 28.02 | C |
| ATOM | 7088 | CH2 | TRP | D | 109 | 35.288 | 8.797 | −35.111 | 1.00 | 29.74 | C |
| ATOM | 7089 | N | THR | D | 110 | 38.323 | 9.800 | −30.157 | 1.00 | 20.99 | N |
| ATOM | 7090 | CA | THR | D | 110 | 37.973 | 11.204 | −30.288 | 1.00 | 24.59 | C |
| ATOM | 7091 | C | THR | D | 110 | 39.140 | 12.072 | −29.841 | 1.00 | 29.06 | C |
| ATOM | 7092 | O | THR | D | 110 | 39.415 | 13.128 | −30.416 | 1.00 | 18.84 | O |
| ATOM | 7093 | CB | THR | D | 110 | 36.748 | 11.524 | −29.400 | 1.00 | 28.97 | C |
| ATOM | 7094 | OG1 | THR | D | 110 | 35.633 | 10.746 | −29.845 | 1.00 | 22.96 | O |
| ATOM | 7095 | CG2 | THR | D | 110 | 36.394 | 13.026 | −29.417 | 1.00 | 15.16 | C |
| ATOM | 7096 | N | SER | D | 111 | 39.810 | 11.603 | −28.793 | 1.00 | 33.82 | N |
| ATOM | 7097 | CA | SER | D | 111 | 40.989 | 12.255 | −28.239 | 1.00 | 30.23 | C |
| ATOM | 7098 | C | SER | D | 111 | 42.120 | 12.395 | −29.257 | 1.00 | 32.09 | C |
| ATOM | 7099 | O | SER | D | 111 | 42.658 | 13.486 | −29.444 | 1.00 | 28.55 | O |
| ATOM | 7100 | CB | SER | D | 111 | 41.491 | 11.461 | −27.037 | 1.00 | 25.25 | C |
| ATOM | 7101 | OG | SER | D | 111 | 40.771 | 11.812 | −25.879 | 1.00 | 44.62 | O |
| ATOM | 7102 | N | LEU | D | 112 | 42.490 | 11.279 | −29.885 | 1.00 | 26.57 | N |
| ATOM | 7103 | CA | LEU | D | 112 | 43.571 | 11.254 | −30.862 | 1.00 | 25.30 | C |
| ATOM | 7104 | C | LEU | D | 112 | 43.193 | 12.118 | −32.063 | 1.00 | 24.23 | C |
| ATOM | 7105 | O | LEU | D | 112 | 44.015 | 12.840 | −32.633 | 1.00 | 17.98 | O |
| ATOM | 7106 | CB | LEU | D | 112 | 43.861 | 9.812 | −31.284 | 1.00 | 17.98 | C |
| ATOM | 7107 | CG | LEU | D | 112 | 44.276 | 8.912 | −30.107 | 1.00 | 25.37 | C |
| ATOM | 7108 | CD1 | LEU | D | 112 | 44.333 | 7.433 | −30.477 | 1.00 | 17.63 | C |
| ATOM | 7109 | CD2 | LEU | D | 112 | 45.602 | 9.359 | −29.529 | 1.00 | 16.55 | C |
| ATOM | 7110 | N | ASP | D | 113 | 41.925 | 12.059 | −32.428 | 1.00 | 18.65 | N |
| ATOM | 7111 | CA | ASP | D | 113 | 41.441 | 12.866 | −33.525 | 1.00 | 20.04 | C |
| ATOM | 7112 | C | ASP | D | 113 | 41.628 | 14.361 | −33.218 | 1.00 | 27.56 | C |
| ATOM | 7113 | O | ASP | D | 113 | 42.108 | 15.120 | −34.062 | 1.00 | 28.79 | O |
| ATOM | 7114 | CB | ASP | D | 113 | 39.990 | 12.518 | −33.791 | 1.00 | 21.17 | C |
| ATOM | 7115 | CG | ASP | D | 113 | 39.465 | 13.123 | −35.065 | 1.00 | 24.14 | C |
| ATOM | 7116 | OD1 | ASP | D | 113 | 40.010 | 14.131 | −35.538 | 1.00 | 24.70 | O |
| ATOM | 7117 | OD2 | ASP | D | 113 | 38.484 | 12.580 | −35.593 | 1.00 | 32.67 | O |
| ATOM | 7118 | N | VAL | D | 114 | 41.270 | 14.772 | −32.003 | 1.00 | 25.29 | N |
| ATOM | 7119 | CA | VAL | D | 114 | 41.441 | 16.153 | −31.558 | 1.00 | 21.34 | C |
| ATOM | 7120 | C | VAL | D | 114 | 42.913 | 16.565 | −31.424 | 1.00 | 22.52 | C |
| ATOM | 7121 | O | VAL | D | 114 | 43.302 | 17.657 | −31.846 | 1.00 | 22.49 | O |
| ATOM | 7122 | CB | VAL | D | 114 | 40.713 | 16.393 | −30.227 | 1.00 | 19.96 | C |
| ATOM | 7123 | CG1 | VAL | D | 114 | 40.865 | 17.838 | −29.777 | 1.00 | 19.99 | C |
| ATOM | 7124 | CG2 | VAL | D | 114 | 39.257 | 16.057 | −30.378 | 1.00 | 25.51 | C |
| ATOM | 7125 | N | LEU | D | 115 | 43.717 | 15.688 | −30.834 | 1.00 | 20.14 | N |
| ATOM | 7126 | CA | LEU | D | 115 | 45.164 | 15.868 | −30.718 | 1.00 | 18.57 | C |
| ATOM | 7127 | C | LEU | D | 115 | 45.832 | 16.238 | −32.034 | 1.00 | 28.13 | C |
| ATOM | 7128 | O | LEU | D | 115 | 46.560 | 17.233 | −32.130 | 1.00 | 25.02 | O |
| ATOM | 7129 | CB | LEU | D | 115 | 45.797 | 14.576 | −30.216 | 1.00 | 19.00 | C |
| ATOM | 7130 | CG | LEU | D | 115 | 47.293 | 14.613 | −29.925 | 1.00 | 27.87 | C |
| ATOM | 7131 | CD1 | LEU | D | 115 | 47.597 | 15.596 | −28.818 | 1.00 | 19.58 | C |
| ATOM | 7132 | CD2 | LEU | D | 115 | 47.791 | 13.221 | −29.574 | 1.00 | 23.65 | C |
| ATOM | 7133 | N | CYS | D | 116 | 45.569 | 15.425 | −33.050 | 1.00 | 30.76 | N |
| ATOM | 7134 | CA | CYS | D | 116 | 46.291 | 15.506 | −34.312 | 1.00 | 23.94 | C |
| ATOM | 7135 | C | CYS | D | 116 | 46.042 | 16.809 | −35.065 | 1.00 | 24.41 | C |
| ATOM | 7136 | O | CYS | D | 116 | 46.985 | 17.419 | −35.570 | 1.00 | 23.74 | O |
| ATOM | 7137 | CB | CYS | D | 116 | 45.968 | 14.286 | −35.176 | 1.00 | 26.28 | C |
| ATOM | 7138 | SG | CYS | D | 116 | 46.752 | 12.770 | −34.572 | 1.00 | 38.72 | S |
| ATOM | 7139 | N | VAL | D | 117 | 44.782 | 17.234 | −35.135 | 1.00 | 19.43 | N |
| ATOM | 7140 | CA | VAL | D | 117 | 44.449 | 18.528 | −35.729 | 1.00 | 19.67 | C |
| ATOM | 7141 | C | VAL | D | 117 | 45.139 | 19.652 | −34.958 | 1.00 | 22.39 | C |
| ATOM | 7142 | O | VAL | D | 117 | 45.689 | 20.576 | −35.546 | 1.00 | 17.92 | O |
| ATOM | 7143 | CB | VAL | D | 117 | 42.927 | 18.790 | −35.714 | 1.00 | 19.31 | C |
| ATOM | 7144 | CG1 | VAL | D | 117 | 42.606 | 20.109 | −36.390 | 1.00 | 18.44 | C |
| ATOM | 7145 | CG2 | VAL | D | 117 | 42.194 | 17.665 | −36.378 | 1.00 | 20.44 | C |
| ATOM | 7146 | N | THR | D | 118 | 45.106 | 19.563 | −33.632 | 1.00 | 26.49 | N |
| ATOM | 7147 | CA | THR | D | 118 | 45.722 | 20.575 | −32.778 | 1.00 | 23.05 | C |
| ATOM | 7148 | C | THR | D | 118 | 47.244 | 20.630 | −32.934 | 1.00 | 21.98 | C |
| ATOM | 7149 | O | THR | D | 118 | 47.825 | 21.704 | −33.113 | 1.00 | 22.16 | O |
| ATOM | 7150 | CB | THR | D | 118 | 45.360 | 20.333 | −31.319 | 1.00 | 22.88 | C |
| ATOM | 7151 | OG1 | THR | D | 118 | 43.933 | 20.317 | −31.188 | 1.00 | 33.77 | O |
| ATOM | 7152 | CG2 | THR | D | 118 | 45.931 | 21.425 | −30.432 | 1.00 | 23.57 | C |
| ATOM | 7153 | N | ALA | D | 119 | 47.892 | 19.476 | −32.881 | 1.00 | 12.26 | N |
| ATOM | 7154 | CA | ALA | D | 119 | 49.331 | 19.443 | −33.069 | 1.00 | 17.27 | C |
| ATOM | 7155 | C | ALA | D | 119 | 49.772 | 19.946 | −34.479 | 1.00 | 26.22 | C |
| ATOM | 7156 | O | ALA | D | 119 | 50.793 | 20.613 | −34.618 | 1.00 | 25.05 | O |
| ATOM | 7157 | CB | ALA | D | 119 | 49.867 | 18.043 | −32.795 | 1.00 | 15.38 | C |
| ATOM | 7158 | N | SER | D | 120 | 49.004 | 19.618 | −35.513 | 1.00 | 18.91 | N |
| ATOM | 7159 | CA | SER | D | 120 | 49.355 | 19.982 | −36.872 | 1.00 | 20.60 | C |
| ATOM | 7160 | C | SER | D | 120 | 49.422 | 21.489 | −37.018 | 1.00 | 24.27 | C |
| ATOM | 7161 | O | SER | D | 120 | 50.432 | 22.041 | −37.471 | 1.00 | 24.52 | O |
| ATOM | 7162 | CB | SER | D | 120 | 48.335 | 19.418 | −37.873 | 1.00 | 17.31 | C |
| ATOM | 7163 | OG | SER | D | 120 | 48.401 | 18.009 | −37.927 | 1.00 | 25.97 | O |
| ATOM | 7164 | N | ILE | D | 121 | 48.334 | 22.146 | −36.637 | 1.00 | 22.68 | N |
| ATOM | 7165 | CA | ILE | D | 121 | 48.193 | 23.580 | −36.824 | 1.00 | 22.41 | C |
| ATOM | 7166 | C | ILE | D | 121 | 49.158 | 24.335 | −35.908 | 1.00 | 21.99 | C |
| ATOM | 7167 | O | ILE | D | 121 | 49.636 | 25.412 | −36.247 | 1.00 | 30.29 | O |
| ATOM | 7168 | CB | ILE | D | 121 | 46.742 | 24.026 | −36.596 | 1.00 | 21.99 | C |
| ATOM | 7169 | CG1 | ILE | D | 121 | 46.613 | 25.544 | −36.720 | 1.00 | 23.48 | C |
| ATOM | 7170 | CG2 | ILE | D | 121 | 46.270 | 23.562 | −35.231 | 1.00 | 27.54 | C |
| ATOM | 7171 | CD1 | ILE | D | 121 | 47.181 | 26.100 | −37.992 | 1.00 | 21.52 | C |
| ATOM | 7172 | N | GLU | D | 122 | 49.472 | 23.768 | −34.757 | 1.00 | 19.88 | N |
| ATOM | 7173 | CA | GLU | D | 122 | 50.502 | 24.374 | −33.931 | 1.00 | 24.82 | C |
| ATOM | 7174 | C | GLU | D | 122 | 51.874 | 24.246 | −34.610 | 1.00 | 27.23 | C |
| ATOM | 7175 | O | GLU | D | 122 | 52.605 | 25.229 | −34.742 | 1.00 | 27.06 | O |
| ATOM | 7176 | CB | GLU | D | 122 | 50.490 | 23.788 | −32.510 | 1.00 | 24.98 | C |
| ATOM | 7177 | CG | GLU | D | 122 | 49.316 | 24.301 | −31.667 | 1.00 | 41.10 | C |
| ATOM | 7178 | CD | GLU | D | 122 | 49.343 | 23.843 | −30.205 | 1.00 | 51.07 | C |
| ATOM | 7179 | OE1 | GLU | D | 122 | 50.446 | 23.723 | −29.627 | 1.00 | 53.01 | O |
| ATOM | 7180 | OE2 | GLU | D | 122 | 48.250 | 23.620 | −29.629 | 1.00 | 44.17 | O |
| ATOM | 7181 | N | THR | D | 123 | 52.208 | 23.040 | −35.060 | 1.00 | 21.46 | N |
| ATOM | 7182 | CA | THR | D | 123 | 53.458 | 22.810 | −35.764 | 1.00 | 18.37 | C |
| ATOM | 7183 | C | THR | D | 123 | 53.606 | 23.733 | −36.978 | 1.00 | 27.70 | C |
| ATOM | 7184 | O | THR | D | 123 | 54.707 | 24.187 | −37.274 | 1.00 | 25.89 | O |
| ATOM | 7185 | CB | THR | D | 123 | 53.606 | 21.333 | −36.189 | 1.00 | 24.89 | C |
| ATOM | 7186 | OG1 | THR | D | 123 | 53.621 | 20.496 | −35.025 | 1.00 | 30.98 | O |
| ATOM | 7187 | CG2 | THR | D | 123 | 54.893 | 21.118 | −36.963 | 1.00 | 24.29 | C |
| ATOM | 7188 | N | LEU | D | 124 | 52.502 | 24.017 | −37.672 | 1.00 | 26.04 | N |
| ATOM | 7189 | CA | LEU | D | 124 | 52.532 | 24.933 | −38.811 | 1.00 | 22.00 | C |
| ATOM | 7190 | C | LEU | D | 124 | 52.848 | 26.360 | −38.383 | 1.00 | 26.62 | C |
| ATOM | 7191 | O | LEU | D | 124 | 53.539 | 27.087 | −39.087 | 1.00 | 22.50 | O |
| ATOM | 7192 | CB | LEU | D | 124 | 51.211 | 24.903 | −39.579 | 1.00 | 24.01 | C |
| ATOM | 7193 | CG | LEU | D | 124 | 50.949 | 23.692 | −40.490 | 1.00 | 28.23 | C |
| ATOM | 7194 | CD1 | LEU | D | 124 | 49.615 | 23.817 | −41.194 | 1.00 | 25.35 | C |
| ATOM | 7195 | CD2 | LEU | D | 124 | 52.055 | 23.506 | −41.504 | 1.00 | 19.24 | C |
| ATOM | 7196 | N | CYS | D | 125 | 52.327 | 26.758 | −37.228 | 1.00 | 29.70 | N |
| ATOM | 7197 | CA | CYS | D | 125 | 52.687 | 28.038 | −36.616 | 1.00 | 31.36 | C |
| ATOM | 7198 | C | CYS | D | 125 | 54.181 | 28.163 | −36.387 | 1.00 | 31.44 | C |
| ATOM | 7199 | O | CYS | D | 125 | 54.802 | 29.149 | −36.779 | 1.00 | 29.80 | O |
| ATOM | 7200 | CB | CYS | D | 125 | 52.003 | 28.188 | −35.268 | 1.00 | 28.98 | C |
| ATOM | 7201 | SG | CYS | D | 125 | 50.426 | 28.943 | −35.374 | 1.00 | 48.66 | S |
| ATOM | 7202 | N | VAL | D | 126 | 54.742 | 27.162 | −35.715 | 1.00 | 28.48 | N |
| ATOM | 7203 | CA | VAL | D | 126 | 56.166 | 27.131 | −35.421 | 1.00 | 29.41 | C |
| ATOM | 7204 | C | VAL | D | 126 | 56.971 | 27.248 | −36.706 | 1.00 | 28.75 | C |
| ATOM | 7205 | O | VAL | D | 126 | 57.924 | 28.005 | −36.770 | 1.00 | 32.07 | O |
| ATOM | 7206 | CB | VAL | D | 126 | 56.560 | 25.844 | −34.669 | 1.00 | 33.33 | C |
| ATOM | 7207 | CG1 | VAL | D | 126 | 58.065 | 25.679 | −34.631 | 1.00 | 26.39 | C |
| ATOM | 7208 | CG2 | VAL | D | 126 | 55.971 | 25.844 | −33.262 | 1.00 | 25.43 | C |
| ATOM | 7209 | N | ILE | D | 127 | 56.576 | 26.503 | −37.732 | 1.00 | 31.64 | N |
| ATOM | 7210 | CA | ILE | D | 127 | 57.242 | 26.573 | −39.029 | 1.00 | 32.65 | C |
| ATOM | 7211 | C | ILE | D | 127 | 57.240 | 28.001 | −39.632 | 1.00 | 33.20 | C |
| ATOM | 7212 | O | ILE | D | 127 | 58.263 | 28.472 | −40.135 | 1.00 | 30.59 | O |
| ATOM | 7213 | CB | ILE | D | 127 | 56.656 | 25.551 | −40.019 | 1.00 | 27.28 | C |
| ATOM | 7214 | CG1 | ILE | D | 127 | 57.021 | 24.132 | −39.587 | 1.00 | 19.26 | C |
| ATOM | 7215 | CG2 | ILE | D | 127 | 57.170 | 25.823 | −41.426 | 1.00 | 25.83 | C |
| ATOM | 7216 | CD1 | ILE | D | 127 | 56.201 | 23.073 | −40.249 | 1.00 | 20.29 | C |
| ATOM | 7217 | N | ALA | D | 128 | 56.107 | 28.691 | −39.569 | 1.00 | 25.46 | N |
| ATOM | 7218 | CA | ALA | D | 128 | 56.051 | 30.083 | −40.011 | 1.00 | 30.35 | C |
| ATOM | 7219 | C | ALA | D | 128 | 56.998 | 30.984 | −39.209 | 1.00 | 40.13 | C |
| ATOM | 7220 | O | ALA | D | 128 | 57.772 | 31.746 | −39.784 | 1.00 | 41.89 | O |
| ATOM | 7221 | CB | ALA | D | 128 | 54.624 | 30.620 | −39.930 | 1.00 | 28.08 | C |
| ATOM | 7222 | N | ILE | D | 129 | 56.919 | 30.897 | −37.882 | 1.00 | 29.49 | N |
| ATOM | 7223 | CA | ILE | D | 129 | 57.730 | 31.724 | −37.000 | 1.00 | 30.99 | C |
| ATOM | 7224 | C | ILE | D | 129 | 59.232 | 31.468 | −37.186 | 1.00 | 37.64 | C |
| ATOM | 7225 | O | ILE | D | 129 | 60.040 | 32.399 | −37.158 | 1.00 | 34.75 | O |
| ATOM | 7226 | CB | ILE | D | 129 | 57.298 | 31.526 | −35.515 | 1.00 | 30.59 | C |
| ATOM | 7227 | CG1 | ILE | D | 129 | 55.998 | 32.279 | −35.242 | 1.00 | 27.46 | C |
| ATOM | 7228 | CG2 | ILE | D | 129 | 58.375 | 31.982 | −34.533 | 1.00 | 18.38 | C |
| ATOM | 7229 | CD1 | ILE | D | 129 | 55.286 | 31.802 | −34.019 | 1.00 | 18.07 | C |
| ATOM | 7230 | N | ASP | D | 130 | 59.584 | 30.201 | −37.384 | 1.00 | 36.33 | N |
| ATOM | 7231 | CA | ASP | D | 130 | 60.962 | 29.762 | −37.568 | 1.00 | 30.99 | C |
| ATOM | 7232 | C | ASP | D | 130 | 61.544 | 30.376 | −38.836 | 1.00 | 37.76 | C |
| ATOM | 7233 | O | ASP | D | 130 | 62.634 | 30.949 | −38.811 | 1.00 | 42.14 | O |
| ATOM | 7234 | CB | ASP | D | 130 | 60.998 | 28.222 | −37.621 | 1.00 | 45.95 | C |
| ATOM | 7235 | CG | ASP | D | 130 | 62.320 | 27.658 | −38.149 | 1.00 | 56.11 | C |
| ATOM | 7236 | OD1 | ASP | D | 130 | 63.341 | 27.696 | −37.415 | 1.00 | 55.76 | O |
| ATOM | 7237 | OD2 | ASP | D | 130 | 62.319 | 27.134 | −39.286 | 1.00 | 41.10 | O |
| ATOM | 7238 | N | ARG | D | 131 | 60.801 | 30.269 | −39.936 | 1.00 | 38.66 | N |
| ATOM | 7239 | CA | ARG | D | 131 | 61.207 | 30.863 | −41.214 | 1.00 | 42.45 | C |
| ATOM | 7240 | C | ARG | D | 131 | 61.283 | 32.389 | −41.153 | 1.00 | 32.35 | C |
| ATOM | 7241 | O | ARG | D | 131 | 62.277 | 32.981 | −41.544 | 1.00 | 46.98 | O |
| ATOM | 7242 | CB | ARG | D | 131 | 60.282 | 30.417 | −42.360 | 1.00 | 32.61 | C |
| ATOM | 7243 | CG | ARG | D | 131 | 60.496 | 28.969 | −42.830 | 1.00 | 36.10 | C |
| ATOM | 7244 | CD | ARG | D | 131 | 61.934 | 28.701 | −43.255 | 1.00 | 39.49 | C |
| ATOM | 7245 | NE | ARG | D | 131 | 62.809 | 28.409 | −42.122 | 1.00 | 45.27 | N |
| ATOM | 7246 | CZ | ARG | D | 131 | 64.139 | 28.452 | −42.171 | 1.00 | 50.57 | C |
| ATOM | 7247 | NH1 | ARG | D | 131 | 64.754 | 28.782 | −43.297 | 1.00 | 59.19 | N |
| ATOM | 7248 | NH2 | ARG | D | 131 | 64.862 | 28.168 | −41.096 | 1.00 | 52.30 | N |
| ATOM | 7249 | N | TYR | D | 132 | 60.233 | 33.027 | −40.668 | 1.00 | 28.95 | N |
| ATOM | 7250 | CA | TYR | D | 132 | 60.274 | 34.468 | −40.440 | 1.00 | 42.35 | C |
| ATOM | 7251 | C | TYR | D | 132 | 61.523 | 34.937 | −39.667 | 1.00 | 50.77 | C |
| ATOM | 7252 | O | TYR | D | 132 | 62.113 | 35.970 | −39.995 | 1.00 | 45.24 | O |
| ATOM | 7253 | CB | TYR | D | 132 | 59.024 | 34.926 | −39.692 | 1.00 | 39.60 | C |
| ATOM | 7254 | CG | TYR | D | 132 | 59.106 | 36.367 | −39.278 | 1.00 | 52.78 | C |
| ATOM | 7255 | CD1 | TYR | D | 132 | 58.761 | 37.384 | −40.167 | 1.00 | 52.73 | C |
| ATOM | 7256 | CD2 | TYR | D | 132 | 59.557 | 36.721 | −38.010 | 1.00 | 51.67 | C |
| ATOM | 7257 | CE1 | TYR | D | 132 | 58.844 | 38.713 | −39.799 | 1.00 | 48.83 | C |
| ATOM | 7258 | CE2 | TYR | D | 132 | 59.643 | 38.051 | −37.631 | 1.00 | 54.01 | C |
| ATOM | 7259 | CZ | TYR | D | 132 | 59.284 | 39.039 | −38.531 | 1.00 | 57.96 | C |
| ATOM | 7260 | OH | TYR | D | 132 | 59.368 | 40.359 | −38.163 | 1.00 | 73.34 | O |
| ATOM | 7261 | N | LEU | D | 133 | 61.904 | 34.193 | −38.629 | 1.00 | 40.20 | N |
| ATOM | 7262 | CA | LEU | D | 133 | 63.051 | 34.564 | −37.818 | 1.00 | 42.77 | C |
| ATOM | 7263 | C | LEU | D | 133 | 64.364 | 34.239 | −38.513 | 1.00 | 54.19 | C |
| ATOM | 7264 | O | LEU | D | 133 | 65.353 | 34.947 | −38.340 | 1.00 | 61.64 | O |
| ATOM | 7265 | CB | LEU | D | 133 | 62.993 | 33.909 | −36.431 | 1.00 | 43.88 | C |
| ATOM | 7266 | CG | LEU | D | 133 | 61.910 | 34.453 | −35.482 | 1.00 | 49.77 | C |
| ATOM | 7267 | CD1 | LEU | D | 133 | 61.963 | 33.745 | −34.150 | 1.00 | 25.18 | C |
| ATOM | 7268 | CD2 | LEU | D | 133 | 62.000 | 35.970 | −35.279 | 1.00 | 40.95 | C |
| ATOM | 7269 | N | ALA | D | 134 | 64.378 | 33.173 | −39.304 | 1.00 | 46.77 | N |
| ATOM | 7270 | CA | ALA | D | 134 | 65.580 | 32.817 | −40.044 | 1.00 | 44.43 | C |
| ATOM | 7271 | C | ALA | D | 134 | 65.869 | 33.844 | −41.139 | 1.00 | 54.38 | C |
| ATOM | 7272 | O | ALA | D | 134 | 66.939 | 33.834 | −41.748 | 1.00 | 59.62 | O |
| ATOM | 7273 | CB | ALA | D | 134 | 65.445 | 31.429 | −40.639 | 1.00 | 34.46 | C |
| ATOM | 7274 | N | ILE | D | 135 | 64.923 | 34.750 | −41.365 | 1.00 | 54.30 | N |
| ATOM | 7275 | CA | ILE | D | 135 | 64.951 | 35.592 | −42.558 | 1.00 | 60.70 | C |
| ATOM | 7276 | C | ILE | D | 135 | 65.088 | 37.084 | −42.260 | 1.00 | 52.86 | C |
| ATOM | 7277 | O | ILE | D | 135 | 65.063 | 37.905 | −43.168 | 1.00 | 61.82 | O |
| ATOM | 7278 | CB | ILE | D | 135 | 63.675 | 35.375 | −43.405 | 1.00 | 52.94 | C |
| ATOM | 7279 | CG1 | ILE | D | 135 | 64.021 | 35.201 | −44.875 | 1.00 | 46.16 | C |
| ATOM | 7280 | CG2 | ILE | D | 135 | 62.691 | 36.519 | −43.215 | 1.00 | 52.57 | C |
| ATOM | 7281 | CD1 | ILE | D | 135 | 62.792 | 35.103 | −45.752 | 1.00 | 62.45 | C |
| ATOM | 7282 | N | THR | D | 136 | 65.229 | 37.436 | −40.992 | 1.00 | 56.84 | N |
| ATOM | 7283 | CA | THR | D | 136 | 65.285 | 38.840 | −40.609 | 1.00 | 60.28 | C |
| ATOM | 7284 | C | THR | D | 136 | 66.332 | 39.052 | −39.522 | 1.00 | 65.78 | C |
| ATOM | 7285 | O | THR | D | 136 | 66.704 | 40.183 | −39.203 | 1.00 | 71.16 | O |
| ATOM | 7286 | CB | THR | D | 136 | 63.902 | 39.353 | −40.118 | 1.00 | 60.24 | C |
| ATOM | 7287 | OG1 | THR | D | 136 | 63.447 | 38.565 | −39.008 | 1.00 | 57.76 | O |
| ATOM | 7288 | CG2 | THR | D | 136 | 62.877 | 39.270 | −41.227 | 1.00 | 55.60 | C |
| ATOM | 7289 | N | SER | D | 137 | 66.803 | 37.947 | −38.959 | 1.00 | 56.23 | N |
| ATOM | 7290 | CA | SER | D | 137 | 67.750 | 37.980 | −37.859 | 1.00 | 60.26 | C |
| ATOM | 7291 | C | SER | D | 137 | 68.614 | 36.738 | −37.969 | 1.00 | 52.35 | C |
| ATOM | 7292 | O | SER | D | 137 | 68.758 | 35.990 | −37.000 | 1.00 | 53.84 | O |
| ATOM | 7293 | CB | SER | D | 137 | 66.998 | 37.976 | −36.525 | 1.00 | 61.61 | C |
| ATOM | 7294 | OG | SER | D | 137 | 65.749 | 38.646 | −36.643 | 1.00 | 53.92 | O |
| ATOM | 7295 | N | PRO | D | 138 | 69.199 | 36.519 | −39.157 | 1.00 | 51.90 | N |
| ATOM | 7296 | CA | PRO | D | 138 | 69.876 | 35.264 | −39.520 | 1.00 | 58.52 | C |
| ATOM | 7297 | C | PRO | D | 138 | 71.044 | 34.922 | −38.592 | 1.00 | 59.59 | C |
| ATOM | 7298 | O | PRO | D | 138 | 71.342 | 33.741 | −38.388 | 1.00 | 50.53 | O |
| ATOM | 7299 | CB | PRO | D | 138 | 70.397 | 35.528 | −40.945 | 1.00 | 42.86 | C |
| ATOM | 7300 | CG | PRO | D | 138 | 69.698 | 36.753 | −41.409 | 1.00 | 44.87 | C |
| ATOM | 7301 | CD | PRO | D | 138 | 69.396 | 37.556 | −40.180 | 1.00 | 55.81 | C |
| ATOM | 7302 | N | PHE | D | 139 | 71.704 | 35.938 | −38.043 | 1.00 | 57.77 | N |
| ATOM | 7303 | CA | PHE | D | 139 | 72.817 | 35.670 | −37.153 | 1.00 | 61.18 | C |
| ATOM | 7304 | C | PHE | D | 139 | 72.277 | 35.055 | −35.888 | 1.00 | 56.87 | C |
| ATOM | 7305 | O | PHE | D | 139 | 72.607 | 33.915 | −35.543 | 1.00 | 51.09 | O |
| ATOM | 7306 | CB | PHE | D | 139 | 73.596 | 36.938 | −36.807 | 1.00 | 64.99 | C |
| ATOM | 7307 | CG | PHE | D | 139 | 74.750 | 36.689 | −35.880 | 1.00 | 63.77 | C |
| ATOM | 7308 | CD1 | PHE | D | 139 | 75.910 | 36.093 | −36.351 | 1.00 | 58.48 | C |
| ATOM | 7309 | CD2 | PHE | D | 139 | 74.665 | 37.022 | −34.533 | 1.00 | 68.97 | C |
| ATOM | 7310 | CE1 | PHE | D | 139 | 76.972 | 35.848 | −35.506 | 1.00 | 65.26 | C |
| ATOM | 7311 | CE2 | PHE | D | 139 | 75.723 | 36.783 | −33.678 | 1.00 | 64.08 | C |
| ATOM | 7312 | CZ | PHE | D | 139 | 76.881 | 36.195 | −34.166 | 1.00 | 72.74 | C |
| ATOM | 7313 | N | ARG | D | 140 | 71.442 | 35.830 | −35.202 | 1.00 | 62.03 | N |
| ATOM | 7314 | CA | ARG | D | 140 | 70.795 | 35.372 | −33.980 | 1.00 | 71.43 | C |
| ATOM | 7315 | C | ARG | D | 140 | 70.083 | 34.047 | −34.229 | 1.00 | 56.54 | C |
| ATOM | 7316 | O | ARG | D | 140 | 69.984 | 33.204 | −33.334 | 1.00 | 51.99 | O |
| ATOM | 7317 | CB | ARG | D | 140 | 69.821 | 36.427 | −33.449 | 1.00 | 68.76 | C |
| ATOM | 7318 | CG | ARG | D | 140 | 70.499 | 37.634 | −32.805 | 1.00 | 74.38 | C |
| ATOM | 7319 | CD | ARG | D | 140 | 69.479 | 38.590 | −32.205 | 1.00 | 94.48 | C |
| ATOM | 7320 | NE | ARG | D | 140 | 70.050 | 39.380 | −31.116 | 1.00 | 116.70 | N |
| ATOM | 7321 | CZ | ARG | D | 140 | 69.338 | 40.148 | −30.295 | 1.00 | 131.79 | C |
| ATOM | 7322 | NH1 | ARG | D | 140 | 68.021 | 40.232 | −30.439 | 1.00 | 125.97 | N |
| ATOM | 7323 | NH2 | ARG | D | 140 | 69.943 | 40.830 | −29.329 | 1.00 | 132.49 | N |
| ATOM | 7324 | N | TYR | D | 141 | 69.608 | 33.856 | −35.453 | 1.00 | 42.68 | N |
| ATOM | 7325 | CA | TYR | D | 141 | 68.998 | 32.590 | −35.803 | 1.00 | 41.47 | C |
| ATOM | 7326 | C | TYR | D | 141 | 70.015 | 31.449 | −35.852 | 1.00 | 50.99 | C |
| ATOM | 7327 | O | TYR | D | 141 | 69.798 | 30.405 | −35.242 | 1.00 | 52.27 | O |
| ATOM | 7328 | CB | TYR | D | 141 | 68.241 | 32.681 | −37.124 | 1.00 | 44.35 | C |
| ATOM | 7329 | CG | TYR | D | 141 | 67.590 | 31.374 | −37.474 | 1.00 | 49.07 | C |
| ATOM | 7330 | CD1 | TYR | D | 141 | 66.262 | 31.130 | −37.159 | 1.00 | 52.39 | C |
| ATOM | 7331 | CD2 | TYR | D | 141 | 68.316 | 30.363 | −38.080 | 1.00 | 50.26 | C |
| ATOM | 7332 | CE1 | TYR | D | 141 | 65.671 | 29.923 | −37.460 | 1.00 | 53.71 | C |
| ATOM | 7333 | CE2 | TYR | D | 141 | 67.738 | 29.156 | −38.384 | 1.00 | 56.28 | C |
| ATOM | 7334 | CZ | TYR | D | 141 | 66.416 | 28.938 | −38.072 | 1.00 | 58.21 | C |
| ATOM | 7335 | OH | TYR | D | 141 | 65.847 | 27.725 | −38.379 | 1.00 | 64.43 | O |
| ATOM | 7336 | N | GLN | D | 142 | 71.116 | 31.636 | −36.579 | 1.00 | 58.51 | N |
| ATOM | 7337 | CA | GLN | D | 142 | 72.141 | 30.591 | −36.677 | 1.00 | 60.44 | C |
| ATOM | 7338 | C | GLN | D | 142 | 72.748 | 30.266 | −35.313 | 1.00 | 54.24 | C |
| ATOM | 7339 | O | GLN | D | 142 | 73.195 | 29.139 | −35.077 | 1.00 | 46.99 | O |
| ATOM | 7340 | CB | GLN | D | 142 | 73.260 | 30.973 | −37.658 | 1.00 | 44.09 | C |
| ATOM | 7341 | CG | GLN | D | 142 | 72.833 | 31.082 | −39.113 | 1.00 | 68.19 | C |
| ATOM | 7342 | CD | GLN | D | 142 | 72.350 | 29.760 | −39.693 | 1.00 | 85.43 | C |
| ATOM | 7343 | OE1 | GLN | D | 142 | 72.414 | 28.719 | −39.034 | 1.00 | 82.31 | O |
| ATOM | 7344 | NE2 | GLN | D | 142 | 71.859 | 29.797 | −40.934 | 1.00 | 82.50 | N |
| ATOM | 7345 | N | SER | D | 143 | 72.765 | 31.249 | −34.417 | 1.00 | 40.62 | N |
| ATOM | 7346 | CA | SER | D | 143 | 73.425 | 31.055 | −33.132 | 1.00 | 56.38 | C |
| ATOM | 7347 | C | SER | D | 143 | 72.526 | 30.467 | −32.038 | 1.00 | 57.58 | C |
| ATOM | 7348 | O | SER | D | 143 | 73.024 | 29.909 | −31.062 | 1.00 | 59.80 | O |
| ATOM | 7349 | CB | SER | D | 143 | 74.105 | 32.346 | −32.653 | 1.00 | 59.15 | C |
| ATOM | 7350 | OG | SER | D | 143 | 73.225 | 33.451 | −32.716 | 1.00 | 63.47 | O |
| ATOM | 7351 | N | LEU | D | 144 | 71.210 | 30.573 | −32.199 | 1.00 | 57.69 | N |
| ATOM | 7352 | CA | LEU | D | 144 | 70.280 | 30.033 | −31.199 | 1.00 | 56.25 | C |
| ATOM | 7353 | C | LEU | D | 144 | 69.652 | 28.703 | −31.598 | 1.00 | 55.07 | C |
| ATOM | 7354 | O | LEU | D | 144 | 69.592 | 27.779 | −30.791 | 1.00 | 46.13 | O |
| ATOM | 7355 | CB | LEU | D | 144 | 69.175 | 31.039 | −30.886 | 1.00 | 44.27 | C |
| ATOM | 7356 | CG | LEU | D | 144 | 69.710 | 32.338 | −30.298 | 1.00 | 60.25 | C |
| ATOM | 7357 | CD1 | LEU | D | 144 | 68.602 | 33.381 | −30.233 | 1.00 | 39.07 | C |
| ATOM | 7358 | CD2 | LEU | D | 144 | 70.346 | 32.083 | −28.930 | 1.00 | 45.81 | C |
| ATOM | 7359 | N | MET | D | 145 | 69.172 | 28.608 | −32.834 | 1.00 | 53.94 | N |
| ATOM | 7360 | CA | MET | D | 145 | 68.469 | 27.405 | −33.263 | 1.00 | 50.03 | C |
| ATOM | 7361 | C | MET | D | 145 | 69.394 | 26.246 | −33.597 | 1.00 | 43.48 | C |
| ATOM | 7362 | O | MET | D | 145 | 70.256 | 26.345 | −34.466 | 1.00 | 56.88 | O |
| ATOM | 7363 | CB | MET | D | 145 | 67.494 | 27.700 | −34.407 | 1.00 | 54.91 | C |
| ATOM | 7364 | CG | MET | D | 145 | 66.151 | 28.273 | −33.915 | 1.00 | 76.96 | C |
| ATOM | 7365 | SD | MET | D | 145 | 64.809 | 27.079 | −33.617 | 1.00 | 67.04 | S |
| ATOM | 7366 | CE | MET | D | 145 | 65.706 | 25.525 | −33.630 | 1.00 | 42.27 | C |
| ATOM | 7367 | N | THR | D | 146 | 69.207 | 25.152 | −32.872 | 1.00 | 34.27 | N |
| ATOM | 7368 | CA | THR | D | 146 | 69.978 | 23.944 | −33.069 | 1.00 | 40.34 | C |
| ATOM | 7369 | C | THR | D | 146 | 68.995 | 22.789 | −33.161 | 1.00 | 37.47 | C |
| ATOM | 7370 | O | THR | D | 146 | 67.806 | 22.971 | −32.936 | 1.00 | 47.72 | O |
| ATOM | 7371 | CB | THR | D | 146 | 70.956 | 23.710 | −31.902 | 1.00 | 44.17 | C |
| ATOM | 7372 | OG1 | THR | D | 146 | 70.225 | 23.464 | −30.691 | 1.00 | 35.16 | O |
| ATOM | 7373 | CG2 | THR | D | 146 | 71.830 | 24.926 | −31.708 | 1.00 | 38.19 | C |
| ATOM | 7374 | N | ARG | D | 147 | 69.477 | 21.603 | −33.489 | 1.00 | 37.23 | N |
| ATOM | 7375 | CA | ARG | D | 147 | 68.595 | 20.457 | −33.583 | 1.00 | 37.98 | C |
| ATOM | 7376 | C | ARG | D | 147 | 68.060 | 20.011 | −32.213 | 1.00 | 41.29 | C |
| ATOM | 7377 | O | ARG | D | 147 | 66.903 | 19.602 | −32.101 | 1.00 | 50.13 | O |
| ATOM | 7378 | CB | ARG | D | 147 | 69.287 | 19.307 | −34.325 | 1.00 | 55.09 | C |
| ATOM | 7379 | CG | ARG | D | 147 | 69.795 | 19.720 | −35.706 | 1.00 | 40.18 | C |
| ATOM | 7380 | CD | ARG | D | 147 | 69.823 | 18.573 | −36.711 | 1.00 | 49.25 | C |
| ATOM | 7381 | NE | ARG | D | 147 | 69.794 | 19.107 | −38.074 | 1.00 | 72.21 | N |
| ATOM | 7382 | CZ | ARG | D | 147 | 70.865 | 19.554 | −38.728 | 1.00 | 77.37 | C |
| ATOM | 7383 | NH1 | ARG | D | 147 | 72.064 | 19.518 | −38.157 | 1.00 | 54.89 | N |
| ATOM | 7384 | NH2 | ARG | D | 147 | 70.741 | 20.033 | −39.959 | 1.00 | 74.69 | N |
| ATOM | 7385 | N | ALA | D | 148 | 68.887 | 20.089 | −31.173 | 1.00 | 38.80 | N |
| ATOM | 7386 | CA | ALA | D | 148 | 68.436 | 19.727 | −29.837 | 1.00 | 38.10 | C |
| ATOM | 7387 | C | ALA | D | 148 | 67.301 | 20.651 | −29.432 | 1.00 | 38.90 | C |
| ATOM | 7388 | O | ALA | D | 148 | 66.313 | 20.225 | −28.824 | 1.00 | 31.80 | O |
| ATOM | 7389 | CB | ALA | D | 148 | 69.577 | 19.817 | −28.846 | 1.00 | 27.14 | C |
| ATOM | 7390 | N | ARG | D | 149 | 67.456 | 21.921 | −29.791 | 1.00 | 33.66 | N |
| ATOM | 7391 | CA | ARG | D | 149 | 66.482 | 22.936 | −29.451 | 1.00 | 26.23 | C |
| ATOM | 7392 | C | ARG | D | 149 | 65.170 | 22.719 | −30.166 | 1.00 | 37.68 | C |
| ATOM | 7393 | O | ARG | D | 149 | 64.095 | 22.741 | −29.548 | 1.00 | 33.18 | O |
| ATOM | 7394 | CB | ARG | D | 149 | 67.027 | 24.328 | −29.740 | 1.00 | 30.30 | C |
| ATOM | 7395 | CG | ARG | D | 149 | 67.270 | 25.101 | −28.461 | 1.00 | 30.50 | C |
| ATOM | 7396 | CD | ARG | D | 149 | 68.158 | 26.307 | −28.612 | 1.00 | 36.58 | C |
| ATOM | 7397 | NE | ARG | D | 149 | 69.106 | 26.336 | −27.505 | 1.00 | 46.07 | N |
| ATOM | 7398 | CZ | ARG | D | 149 | 70.135 | 27.170 | −27.410 | 1.00 | 54.25 | C |
| ATOM | 7399 | NH1 | ARG | D | 149 | 70.351 | 28.079 | −28.353 | 1.00 | 56.94 | N |
| ATOM | 7400 | NH2 | ARG | D | 149 | 70.950 | 27.098 | −26.363 | 1.00 | 55.80 | N |
| ATOM | 7401 | N | ALA | D | 150 | 65.261 | 22.516 | −31.476 | 1.00 | 34.90 | N |
| ATOM | 7402 | CA | ALA | D | 150 | 64.085 | 22.241 | −32.284 | 1.00 | 27.49 | C |
| ATOM | 7403 | C | ALA | D | 150 | 63.256 | 21.081 | −31.717 | 1.00 | 30.80 | C |
| ATOM | 7404 | O | ALA | D | 150 | 62.028 | 21.166 | −31.664 | 1.00 | 28.73 | O |
| ATOM | 7405 | CB | ALA | D | 150 | 64.498 | 21.958 | −33.703 | 1.00 | 27.97 | C |
| ATOM | 7406 | N | LYS | D | 151 | 63.928 | 20.008 | −31.296 | 1.00 | 25.88 | N |
| ATOM | 7407 | CA | LYS | D | 151 | 63.256 | 18.862 | −30.683 | 1.00 | 33.27 | C |
| ATOM | 7408 | C | LYS | D | 151 | 62.529 | 19.238 | −29.382 | 1.00 | 37.25 | C |
| ATOM | 7409 | O | LYS | D | 151 | 61.414 | 18.771 | −29.106 | 1.00 | 34.39 | O |
| ATOM | 7410 | CB | LYS | D | 151 | 64.247 | 17.720 | −30.433 | 1.00 | 31.45 | C |
| ATOM | 7411 | CG | LYS | D | 151 | 64.693 | 17.047 | −31.714 | 1.00 | 45.16 | C |
| ATOM | 7412 | CD | LYS | D | 151 | 65.653 | 15.899 | −31.466 | 1.00 | 57.07 | C |
| ATOM | 7413 | CE | LYS | D | 151 | 66.528 | 15.659 | −32.691 | 1.00 | 52.60 | C |
| ATOM | 7414 | NZ | LYS | D | 151 | 67.276 | 14.371 | −32.612 | 1.00 | 71.61 | N |
| ATOM | 7415 | N | VAL | D | 152 | 63.160 | 20.081 | −28.580 | 1.00 | 23.31 | N |
| ATOM | 7416 | CA | VAL | D | 152 | 62.483 | 20.608 | −27.411 | 1.00 | 29.69 | C |
| ATOM | 7417 | C | VAL | D | 152 | 61.190 | 21.309 | −27.846 | 1.00 | 28.57 | C |
| ATOM | 7418 | O | VAL | D | 152 | 60.129 | 21.049 | −27.286 | 1.00 | 34.22 | O |
| ATOM | 7419 | CB | VAL | D | 152 | 63.409 | 21.533 | −26.581 | 1.00 | 25.80 | C |
| ATOM | 7420 | CG1 | VAL | D | 152 | 62.619 | 22.376 | −25.607 | 1.00 | 17.42 | C |
| ATOM | 7421 | CG2 | VAL | D | 152 | 64.415 | 20.701 | −25.846 | 1.00 | 25.18 | C |
| ATOM | 7422 | N | ILE | D | 153 | 61.273 | 22.165 | −28.863 | 1.00 | 24.55 | N |
| ATOM | 7423 | CA | ILE | D | 153 | 60.098 | 22.876 | −29.370 | 1.00 | 24.31 | C |
| ATOM | 7424 | C | ILE | D | 153 | 58.995 | 21.941 | −29.887 | 1.00 | 25.84 | C |
| ATOM | 7425 | O | ILE | D | 153 | 57.814 | 22.159 | −29.618 | 1.00 | 25.79 | O |
| ATOM | 7426 | CB | ILE | D | 153 | 60.471 | 23.871 | −30.478 | 1.00 | 26.58 | C |
| ATOM | 7427 | CG1 | ILE | D | 153 | 61.446 | 24.929 | −29.951 | 1.00 | 21.99 | C |
| ATOM | 7428 | CG2 | ILE | D | 153 | 59.222 | 24.525 | −31.045 | 1.00 | 18.33 | C |
| ATOM | 7429 | CD1 | ILE | D | 153 | 62.353 | 25.523 | −31.018 | 1.00 | 19.47 | C |
| ATOM | 7430 | N | ILE | D | 154 | 59.380 | 20.906 | −30.629 | 1.00 | 23.16 | N |
| ATOM | 7431 | CA | ILE | D | 154 | 58.435 | 19.889 | −31.080 | 1.00 | 23.89 | C |
| ATOM | 7432 | C | ILE | D | 154 | 57.636 | 19.300 | −29.909 | 1.00 | 30.42 | C |
| ATOM | 7433 | O | ILE | D | 154 | 56.410 | 19.194 | −29.975 | 1.00 | 26.57 | O |
| ATOM | 7434 | CB | ILE | D | 154 | 59.158 | 18.765 | −31.859 | 1.00 | 27.64 | C |
| ATOM | 7435 | CG1 | ILE | D | 154 | 59.411 | 19.221 | −33.292 | 1.00 | 29.29 | C |
| ATOM | 7436 | CG2 | ILE | D | 154 | 58.352 | 17.464 | −31.860 | 1.00 | 18.05 | C |
| ATOM | 7437 | CD1 | ILE | D | 154 | 60.532 | 18.483 | −33.985 | 1.00 | 40.50 | C |
| ATOM | 7438 | N | CYS | D | 155 | 58.336 | 18.939 | −28.835 | 1.00 | 25.15 | N |
| ATOM | 7439 | CA | CYS | D | 155 | 57.715 | 18.260 | −27.701 | 1.00 | 28.64 | C |
| ATOM | 7440 | C | CYS | D | 155 | 56.866 | 19.198 | −26.862 | 1.00 | 27.82 | C |
| ATOM | 7441 | O | CYS | D | 155 | 55.849 | 18.796 | −26.298 | 1.00 | 20.58 | O |
| ATOM | 7442 | CB | CYS | D | 155 | 58.781 | 17.604 | −26.824 | 1.00 | 45.98 | C |
| ATOM | 7443 | SG | CYS | D | 155 | 59.798 | 16.403 | −27.707 | 1.00 | 65.51 | S |
| ATOM | 7444 | N | THR | D | 156 | 57.309 | 20.444 | −26.763 | 1.00 | 26.79 | N |
| ATOM | 7445 | CA | THR | D | 156 | 56.534 | 21.479 | −26.101 | 1.00 | 28.92 | C |
| ATOM | 7446 | C | THR | D | 156 | 55.198 | 21.612 | −26.829 | 1.00 | 23.27 | C |
| ATOM | 7447 | O | THR | D | 156 | 54.148 | 21.678 | −26.198 | 1.00 | 19.47 | O |
| ATOM | 7448 | CB | THR | D | 156 | 57.314 | 22.814 | −26.092 | 1.00 | 23.83 | C |
| ATOM | 7449 | OG1 | THR | D | 156 | 58.552 | 22.621 | −25.408 | 1.00 | 30.54 | O |
| ATOM | 7450 | CG2 | THR | D | 156 | 56.548 | 23.909 | −25.395 | 1.00 | 17.72 | C |
| ATOM | 7451 | N | VAL | D | 157 | 55.251 | 21.620 | −28.159 | 1.00 | 22.30 | N |
| ATOM | 7452 | CA | VAL | D | 157 | 54.044 | 21.641 | −28.984 | 1.00 | 20.58 | C |
| ATOM | 7453 | C | VAL | D | 157 | 53.124 | 20.439 | −28.763 | 1.00 | 20.63 | C |
| ATOM | 7454 | O | VAL | D | 157 | 51.934 | 20.615 | −28.549 | 1.00 | 24.08 | O |
| ATOM | 7455 | CB | VAL | D | 157 | 54.381 | 21.752 | −30.461 | 1.00 | 17.06 | C |
| ATOM | 7456 | CG1 | VAL | D | 157 | 53.241 | 21.228 | −31.292 | 1.00 | 27.89 | C |
| ATOM | 7457 | CG2 | VAL | D | 157 | 54.668 | 23.190 | −30.818 | 1.00 | 22.07 | C |
| ATOM | 7458 | N | TRP | D | 158 | 53.668 | 19.226 | −28.802 | 1.00 | 20.87 | N |
| ATOM | 7459 | CA | TRP | D | 158 | 52.888 | 18.028 | −28.503 | 1.00 | 19.71 | C |
| ATOM | 7460 | C | TRP | D | 158 | 52.324 | 18.005 | −27.058 | 1.00 | 27.18 | C |
| ATOM | 7461 | O | TRP | D | 158 | 51.195 | 17.548 | −26.819 | 1.00 | 21.35 | O |
| ATOM | 7462 | CB | TRP | D | 158 | 53.708 | 16.767 | −28.794 | 1.00 | 18.69 | C |
| ATOM | 7463 | CG | TRP | D | 158 | 53.765 | 16.424 | −30.259 | 1.00 | 33.87 | C |
| ATOM | 7464 | CD1 | TRP | D | 158 | 54.735 | 16.786 | −31.154 | 1.00 | 31.36 | C |
| ATOM | 7465 | CD2 | TRP | D | 158 | 52.808 | 15.655 | −31.000 | 1.00 | 35.65 | C |
| ATOM | 7466 | NE1 | TRP | D | 158 | 54.439 | 16.296 | −32.402 | 1.00 | 25.97 | N |
| ATOM | 7467 | CE2 | TRP | D | 158 | 53.262 | 15.600 | −32.339 | 1.00 | 30.33 | C |
| ATOM | 7468 | CE3 | TRP | D | 158 | 51.610 | 15.013 | −30.664 | 1.00 | 30.95 | C |
| ATOM | 7469 | CZ2 | TRP | D | 158 | 52.564 | 14.926 | −33.340 | 1.00 | 30.21 | C |
| ATOM | 7470 | CZ3 | TRP | D | 158 | 50.917 | 14.346 | −31.662 | 1.00 | 41.29 | C |
| ATOM | 7471 | CH2 | TRP | D | 158 | 51.400 | 14.308 | −32.989 | 1.00 | 32.18 | C |
| ATOM | 7472 | N | ALA | D | 159 | 53.114 | 18.495 | −26.102 | 1.00 | 26.63 | N |
| ATOM | 7473 | CA | ALA | D | 159 | 52.671 | 18.640 | −24.721 | 1.00 | 17.00 | C |
| ATOM | 7474 | C | ALA | D | 159 | 51.461 | 19.554 | −24.621 | 1.00 | 22.89 | C |
| ATOM | 7475 | O | ALA | D | 159 | 50.405 | 19.148 | −24.128 | 1.00 | 26.91 | O |
| ATOM | 7476 | CB | ALA | D | 159 | 53.780 | 19.196 | −23.880 | 1.00 | 20.16 | C |
| ATOM | 7477 | N | ILE | D | 160 | 51.622 | 20.796 | −25.068 | 1.00 | 17.90 | N |
| ATOM | 7478 | CA | ILE | D | 160 | 50.525 | 21.754 | −25.083 | 1.00 | 20.21 | C |
| ATOM | 7479 | C | ILE | D | 160 | 49.311 | 21.162 | −25.774 | 1.00 | 28.21 | C |
| ATOM | 7480 | O | ILE | D | 160 | 48.170 | 21.398 | −25.367 | 1.00 | 26.79 | O |
| ATOM | 7481 | CB | ILE | D | 160 | 50.908 | 23.044 | −25.798 | 1.00 | 25.44 | C |
| ATOM | 7482 | CG1 | ILE | D | 160 | 51.961 | 23.787 | −24.971 | 1.00 | 28.15 | C |
| ATOM | 7483 | CG2 | ILE | D | 160 | 49.678 | 23.902 | −26.028 | 1.00 | 18.32 | C |
| ATOM | 7484 | CD1 | ILE | D | 160 | 52.618 | 24.945 | −25.693 | 1.00 | 22.66 | C |
| ATOM | 7485 | N | SER | D | 161 | 49.562 | 20.376 | −26.813 | 1.00 | 24.17 | N |
| ATOM | 7486 | CA | SER | D | 161 | 48.483 | 19.755 | −27.565 | 1.00 | 24.04 | C |
| ATOM | 7487 | C | SER | D | 161 | 47.699 | 18.706 | −26.774 | 1.00 | 19.64 | C |
| ATOM | 7488 | O | SER | D | 161 | 46.482 | 18.756 | −26.764 | 1.00 | 26.32 | O |
| ATOM | 7489 | CB | SER | D | 161 | 49.010 | 19.184 | −28.873 | 1.00 | 28.01 | C |
| ATOM | 7490 | OG | SER | D | 161 | 49.485 | 20.230 | −29.702 | 1.00 | 36.67 | O |
| ATOM | 7491 | N | ALA | D | 162 | 48.376 | 17.758 | −26.130 | 1.00 | 18.53 | N |
| ATOM | 7492 | CA | ALA | D | 162 | 47.695 | 16.808 | −25.244 | 1.00 | 25.97 | C |
| ATOM | 7493 | C | ALA | D | 162 | 46.943 | 17.508 | −24.075 | 1.00 | 30.96 | C |
| ATOM | 7494 | O | ALA | D | 162 | 45.828 | 17.119 | −23.696 | 1.00 | 22.01 | O |
| ATOM | 7495 | CB | ALA | D | 162 | 48.681 | 15.782 | −24.712 | 1.00 | 17.14 | C |
| ATOM | 7496 | N | LEU | D | 163 | 47.565 | 18.536 | −23.505 | 1.00 | 21.69 | N |
| ATOM | 7497 | CA | LEU | D | 163 | 46.905 | 19.377 | −22.523 | 1.00 | 25.12 | C |
| ATOM | 7498 | C | LEU | D | 163 | 45.494 | 19.829 | −22.939 | 1.00 | 24.24 | C |
| ATOM | 7499 | O | LEU | D | 163 | 44.526 | 19.517 | −22.262 | 1.00 | 22.72 | O |
| ATOM | 7500 | CB | LEU | D | 163 | 47.761 | 20.609 | −22.237 | 1.00 | 28.83 | C |
| ATOM | 7501 | CG | LEU | D | 163 | 47.265 | 21.453 | −21.067 | 1.00 | 19.49 | C |
| ATOM | 7502 | CD1 | LEU | D | 163 | 47.125 | 20.550 | −19.865 | 1.00 | 14.86 | C |
| ATOM | 7503 | CD2 | LEU | D | 163 | 48.207 | 22.610 | −20.795 | 1.00 | 15.19 | C |
| ATOM | 7504 | N | VAL | D | 164 | 45.389 | 20.581 | −24.038 | 1.00 | 29.89 | N |
| ATOM | 7505 | CA | VAL | D | 164 | 44.100 | 21.124 | −24.501 | 1.00 | 33.15 | C |
| ATOM | 7506 | C | VAL | D | 164 | 43.209 | 20.125 | −25.259 | 1.00 | 28.57 | C |
| ATOM | 7507 | O | VAL | D | 164 | 42.114 | 20.490 | −25.702 | 1.00 | 31.65 | O |
| ATOM | 7508 | CB | VAL | D | 164 | 44.263 | 22.392 | −25.387 | 1.00 | 28.92 | C |
| ATOM | 7509 | CG1 | VAL | D | 164 | 45.253 | 23.344 | −24.776 | 1.00 | 19.27 | C |
| ATOM | 7510 | CG2 | VAL | D | 164 | 44.671 | 22.015 | −26.816 | 1.00 | 20.60 | C |
| ATOM | 7511 | N | SER | D | 165 | 43.665 | 18.881 | −25.401 | 1.00 | 19.24 | N |
| ATOM | 7512 | CA | SER | D | 165 | 42.882 | 17.870 | −26.103 | 1.00 | 19.68 | C |
| ATOM | 7513 | C | SER | D | 165 | 42.556 | 16.605 | −25.293 | 1.00 | 27.11 | C |
| ATOM | 7514 | O | SER | D | 165 | 41.390 | 16.217 | −25.195 | 1.00 | 38.00 | O |
| ATOM | 7515 | CB | SER | D | 165 | 43.540 | 17.509 | −27.428 | 1.00 | 26.61 | C |
| ATOM | 7516 | OG | SER | D | 165 | 44.804 | 16.934 | −27.195 | 1.00 | 42.66 | O |
| ATOM | 7517 | N | PHE | D | 166 | 43.558 | 15.956 | −24.712 | 1.00 | 25.41 | N |
| ATOM | 7518 | CA | PHE | D | 166 | 43.291 | 14.833 | −23.805 | 1.00 | 32.72 | C |
| ATOM | 7519 | C | PHE | D | 166 | 42.580 | 15.218 | −22.501 | 1.00 | 32.78 | C |
| ATOM | 7520 | O | PHE | D | 166 | 41.643 | 14.552 | −22.063 | 1.00 | 28.27 | O |
| ATOM | 7521 | CB | PHE | D | 166 | 44.587 | 14.132 | −23.426 | 1.00 | 34.98 | C |
| ATOM | 7522 | CG | PHE | D | 166 | 45.151 | 13.306 | −24.508 | 1.00 | 37.78 | C |
| ATOM | 7523 | CD1 | PHE | D | 166 | 46.526 | 13.128 | −24.615 | 1.00 | 37.93 | C |
| ATOM | 7524 | CD2 | PHE | D | 166 | 44.312 | 12.710 | −25.432 | 1.00 | 27.58 | C |
| ATOM | 7525 | CE1 | PHE | D | 166 | 47.054 | 12.347 | −25.630 | 1.00 | 48.10 | C |
| ATOM | 7526 | CE2 | PHE | D | 166 | 44.823 | 11.929 | −26.445 | 1.00 | 36.47 | C |
| ATOM | 7527 | CZ | PHE | D | 166 | 46.196 | 11.746 | −26.553 | 1.00 | 48.52 | C |
| ATOM | 7528 | N | LEU | D | 167 | 43.050 | 16.284 | −21.869 | 1.00 | 31.22 | N |
| ATOM | 7529 | CA | LEU | D | 167 | 42.644 | 16.578 | −20.507 | 1.00 | 24.27 | C |
| ATOM | 7530 | C | LEU | D | 167 | 41.205 | 17.030 | −20.286 | 1.00 | 22.52 | C |
| ATOM | 7531 | O | LEU | D | 167 | 40.596 | 16.625 | −19.310 | 1.00 | 31.01 | O |
| ATOM | 7532 | CB | LEU | D | 167 | 43.650 | 17.505 | −19.837 | 1.00 | 29.06 | C |
| ATOM | 7533 | CG | LEU | D | 167 | 44.659 | 16.667 | −19.043 | 1.00 | 32.36 | C |
| ATOM | 7534 | CD1 | LEU | D | 167 | 45.995 | 17.351 | −18.961 | 1.00 | 28.14 | C |
| ATOM | 7535 | CD2 | LEU | D | 167 | 44.103 | 16.366 | −17.655 | 1.00 | 30.10 | C |
| ATOM | 7536 | N | PRO | D | 168 | 40.654 | 17.871 | −21.172 | 1.00 | 27.00 | N |
| ATOM | 7537 | CA | PRO | D | 168 | 39.216 | 18.153 | −21.034 | 1.00 | 25.38 | C |
| ATOM | 7538 | C | PRO | D | 168 | 38.318 | 16.939 | −21.346 | 1.00 | 27.51 | C |
| ATOM | 7539 | O | PRO | D | 168 | 37.169 | 16.876 | −20.891 | 1.00 | 23.43 | O |
| ATOM | 7540 | CB | PRO | D | 168 | 38.986 | 19.276 | −22.041 | 1.00 | 20.69 | C |
| ATOM | 7541 | CG | PRO | D | 168 | 40.330 | 19.950 | −22.144 | 1.00 | 25.76 | C |
| ATOM | 7542 | CD | PRO | D | 168 | 41.310 | 18.819 | −22.089 | 1.00 | 27.16 | C |
| ATOM | 7543 | N | ILE | D | 169 | 38.840 | 15.975 | −22.098 | 1.00 | 22.26 | N |
| ATOM | 7544 | CA | ILE | D | 169 | 38.081 | 14.772 | −22.374 | 1.00 | 19.56 | C |
| ATOM | 7545 | C | ILE | D | 169 | 38.069 | 13.833 | −21.179 | 1.00 | 23.23 | C |
| ATOM | 7546 | O | ILE | D | 169 | 37.032 | 13.274 | −20.832 | 1.00 | 29.55 | O |
| ATOM | 7547 | CB | ILE | D | 169 | 38.552 | 14.068 | −23.660 | 1.00 | 27.64 | C |
| ATOM | 7548 | CG1 | ILE | D | 169 | 38.224 | 14.961 | −24.858 | 1.00 | 27.07 | C |
| ATOM | 7549 | CG2 | ILE | D | 169 | 37.887 | 12.701 | −23.820 | 1.00 | 20.45 | C |
| ATOM | 7550 | CD1 | ILE | D | 169 | 37.779 | 14.209 | −26.070 | 1.00 | 33.78 | C |
| ATOM | 7551 | N | MET | D | 170 | 39.208 | 13.676 | −20.523 | 1.00 | 29.38 | N |
| ATOM | 7552 | CA | MET | D | 170 | 39.241 | 12.841 | −19.324 | 1.00 | 31.28 | C |
| ATOM | 7553 | C | MET | D | 170 | 38.500 | 13.438 | −18.100 | 1.00 | 30.64 | C |
| ATOM | 7554 | O | MET | D | 170 | 38.050 | 12.701 | −17.221 | 1.00 | 33.24 | O |
| ATOM | 7555 | CB | MET | D | 170 | 40.671 | 12.335 | −19.009 | 1.00 | 30.31 | C |
| ATOM | 7556 | CG | MET | D | 170 | 41.823 | 13.274 | −19.351 | 1.00 | 31.99 | C |
| ATOM | 7557 | SD | MET | D | 170 | 43.429 | 12.470 | −19.662 | 1.00 | 46.70 | S |
| ATOM | 7558 | CE | MET | D | 170 | 43.507 | 11.146 | −18.459 | 1.00 | 21.11 | C |
| ATOM | 7559 | N | MET | D | 171 | 38.340 | 14.760 | −18.062 | 1.00 | 27.69 | N |
| ATOM | 7560 | CA | MET | D | 171 | 37.560 | 15.401 | −17.003 | 1.00 | 24.80 | C |
| ATOM | 7561 | C | MET | D | 171 | 36.121 | 15.645 | −17.447 | 1.00 | 30.85 | C |
| ATOM | 7562 | O | MET | D | 171 | 35.359 | 16.342 | −16.774 | 1.00 | 28.17 | O |
| ATOM | 7563 | CB | MET | D | 171 | 38.204 | 16.717 | −16.594 | 1.00 | 23.05 | C |
| ATOM | 7564 | CG | MET | D | 171 | 39.607 | 16.556 | −16.038 | 1.00 | 30.00 | C |
| ATOM | 7565 | SD | MET | D | 171 | 40.415 | 18.164 | −15.938 | 1.00 | 36.62 | S |
| ATOM | 7566 | CE | MET | D | 171 | 39.413 | 18.900 | −14.646 | 1.00 | 67.55 | C |
| ATOM | 7567 | N | HIS | D | 172 | 35.765 | 15.090 | −18.599 | 1.00 | 25.40 | N |
| ATOM | 7568 | CA | HIS | D | 172 | 34.403 | 15.170 | −19.111 | 1.00 | 26.32 | C |
| ATOM | 7569 | C | HIS | D | 172 | 33.864 | 16.588 | −19.319 | 1.00 | 25.68 | C |
| ATOM | 7570 | O | HIS | D | 172 | 32.661 | 16.804 | −19.260 | 1.00 | 28.54 | O |
| ATOM | 7571 | CB | HIS | D | 172 | 33.465 | 14.387 | −18.201 | 1.00 | 22.45 | C |
| ATOM | 7572 | CG | HIS | D | 172 | 33.946 | 13.005 | −17.879 | 1.00 | 26.46 | C |
| ATOM | 7573 | ND1 | HIS | D | 172 | 33.427 | 11.879 | −18.483 | 1.00 | 24.98 | N |
| ATOM | 7574 | CD2 | HIS | D | 172 | 34.884 | 12.563 | −17.010 | 1.00 | 28.11 | C |
| ATOM | 7575 | CE1 | HIS | D | 172 | 34.031 | 10.808 | −18.006 | 1.00 | 24.39 | C |
| ATOM | 7576 | NE2 | HIS | D | 172 | 34.917 | 11.191 | −17.104 | 1.00 | 23.42 | N |
| ATOM | 7577 | N | TRP | D | 173 | 34.746 | 17.541 | −19.601 | 1.00 | 25.47 | N |
| ATOM | 7578 | CA | TRP | D | 173 | 34.342 | 18.939 | −19.746 | 1.00 | 26.74 | C |
| ATOM | 7579 | C | TRP | D | 173 | 33.500 | 19.165 | −20.988 | 1.00 | 29.81 | C |
| ATOM | 7580 | O | TRP | D | 173 | 32.887 | 20.217 | −21.157 | 1.00 | 33.48 | O |
| ATOM | 7581 | CB | TRP | D | 173 | 35.573 | 19.839 | −19.800 | 1.00 | 24.06 | C |
| ATOM | 7582 | CG | TRP | D | 173 | 36.233 | 20.044 | −18.466 | 1.00 | 28.36 | C |
| ATOM | 7583 | CD1 | TRP | D | 173 | 36.002 | 19.343 | −17.311 | 1.00 | 22.02 | C |
| ATOM | 7584 | CD2 | TRP | D | 173 | 37.255 | 21.004 | −18.157 | 1.00 | 28.26 | C |
| ATOM | 7585 | NE1 | TRP | D | 173 | 36.805 | 19.825 | −16.303 | 1.00 | 21.74 | N |
| ATOM | 7586 | CE2 | TRP | D | 173 | 37.582 | 20.843 | −16.797 | 1.00 | 27.98 | C |
| ATOM | 7587 | CE3 | TRP | D | 173 | 37.918 | 21.989 | −18.896 | 1.00 | 23.06 | C |
| ATOM | 7588 | CZ2 | TRP | D | 173 | 38.548 | 21.627 | −16.166 | 1.00 | 24.59 | C |
| ATOM | 7589 | CZ3 | TRP | D | 173 | 38.872 | 22.766 | −18.263 | 1.00 | 25.97 | C |
| ATOM | 7590 | CH2 | TRP | D | 173 | 39.176 | 22.582 | −16.916 | 1.00 | 19.52 | C |
| ATOM | 7591 | N | TRP | D | 174 | 33.463 | 18.163 | −21.854 | 1.00 | 26.71 | N |
| ATOM | 7592 | CA | TRP | D | 174 | 32.848 | 18.315 | −23.161 | 1.00 | 23.52 | C |
| ATOM | 7593 | C | TRP | D | 174 | 31.373 | 17.942 | −23.168 | 1.00 | 29.29 | C |
| ATOM | 7594 | O | TRP | D | 174 | 30.632 | 18.357 | −24.063 | 1.00 | 29.53 | O |
| ATOM | 7595 | CB | TRP | D | 174 | 33.585 | 17.458 | −24.183 | 1.00 | 22.03 | C |
| ATOM | 7596 | CG | TRP | D | 174 | 33.575 | 16.004 | −23.841 | 1.00 | 22.53 | C |
| ATOM | 7597 | CD1 | TRP | D | 174 | 34.394 | 15.370 | −22.956 | 1.00 | 22.59 | C |
| ATOM | 7598 | CD2 | TRP | D | 174 | 32.699 | 15.001 | −24.367 | 1.00 | 20.26 | C |
| ATOM | 7599 | NE1 | TRP | D | 174 | 34.088 | 14.032 | −22.900 | 1.00 | 19.03 | N |
| ATOM | 7600 | CE2 | TRP | D | 174 | 33.051 | 13.779 | −23.756 | 1.00 | 17.62 | C |
| ATOM | 7601 | CE3 | TRP | D | 174 | 31.651 | 15.017 | −25.287 | 1.00 | 20.15 | C |
| ATOM | 7602 | CZ2 | TRP | D | 174 | 32.407 | 12.588 | −24.048 | 1.00 | 17.06 | C |
| ATOM | 7603 | CZ3 | TRP | D | 174 | 31.013 | 13.830 | −25.573 | 1.00 | 21.16 | C |
| ATOM | 7604 | CH2 | TRP | D | 174 | 31.396 | 12.631 | −24.959 | 1.00 | 16.11 | C |
| ATOM | 7605 | N | ARG | D | 175 | 30.944 | 17.165 | −22.175 | 1.00 | 30.92 | N |
| ATOM | 7606 | CA | ARG | D | 175 | 29.594 | 16.576 | −22.197 | 1.00 | 34.78 | C |
| ATOM | 7607 | C | ARG | D | 175 | 28.437 | 17.574 | −22.066 | 1.00 | 34.17 | C |
| ATOM | 7608 | O | ARG | D | 175 | 28.567 | 18.617 | −21.431 | 1.00 | 28.59 | O |
| ATOM | 7609 | CB | ARG | D | 175 | 29.467 | 15.468 | −21.152 | 1.00 | 17.96 | C |
| ATOM | 7610 | CG | ARG | D | 175 | 30.408 | 14.321 | −21.395 | 1.00 | 18.10 | C |
| ATOM | 7611 | CD | ARG | D | 175 | 30.020 | 13.142 | −20.554 | 1.00 | 23.60 | C |
| ATOM | 7612 | NE | ARG | D | 175 | 31.094 | 12.168 | −20.400 | 1.00 | 23.40 | N |
| ATOM | 7613 | CZ | ARG | D | 175 | 31.252 | 11.094 | −21.172 | 1.00 | 26.93 | C |
| ATOM | 7614 | NH1 | ARG | D | 175 | 30.406 | 10.861 | −22.165 | 1.00 | 28.77 | N |
| ATOM | 7615 | NH2 | ARG | D | 175 | 32.255 | 10.252 | −20.954 | 1.00 | 24.08 | N |
| ATOM | 7616 | N | ASP | D | 176 | 27.304 | 17.238 | −22.682 | 1.00 | 39.51 | N |
| ATOM | 7617 | CA | ASP | D | 176 | 26.106 | 18.063 | −22.584 | 1.00 | 39.91 | C |
| ATOM | 7618 | C | ASP | D | 176 | 25.069 | 17.473 | −21.617 | 1.00 | 34.58 | C |
| ATOM | 7619 | O | ASP | D | 176 | 25.210 | 16.347 | −21.137 | 1.00 | 30.48 | O |
| ATOM | 7620 | CB | ASP | D | 176 | 25.494 | 18.285 | −23.967 | 1.00 | 38.79 | C |
| ATOM | 7621 | CG | ASP | D | 176 | 24.764 | 19.625 | −24.082 | 1.00 | 59.31 | C |
| ATOM | 7622 | OD1 | ASP | D | 176 | 24.712 | 20.390 | −23.089 | 1.00 | 51.38 | O |
| ATOM | 7623 | OD2 | ASP | D | 176 | 24.245 | 19.914 | −25.180 | 1.00 | 65.27 | O |
| ATOM | 7624 | N | GLU | D | 177 | 24.035 | 18.252 | −21.322 | 1.00 | 43.89 | N |
| ATOM | 7625 | CA | GLU | D | 177 | 22.994 | 17.810 | −20.402 | 1.00 | 49.30 | C |
| ATOM | 7626 | C | GLU | D | 177 | 21.768 | 17.206 | −21.126 | 1.00 | 50.47 | C |
| ATOM | 7627 | O | GLU | D | 177 | 21.092 | 16.340 | −20.569 | 1.00 | 49.84 | O |
| ATOM | 7628 | CB | GLU | D | 177 | 22.579 | 18.949 | −19.461 | 1.00 | 47.65 | C |
| ATOM | 7629 | CG | GLU | D | 177 | 23.735 | 19.637 | −18.725 | 1.00 | 55.47 | C |
| ATOM | 7630 | CD | GLU | D | 177 | 24.302 | 18.807 | −17.573 | 1.00 | 82.15 | C |
| ATOM | 7631 | OE1 | GLU | D | 177 | 23.901 | 17.626 | −17.430 | 1.00 | 81.01 | O |
| ATOM | 7632 | OE2 | GLU | D | 177 | 25.148 | 19.338 | −16.813 | 1.00 | 59.73 | O |
| ATOM | 7633 | N | ASP | D | 178 | 21.507 | 17.646 | −22.360 | 1.00 | 46.36 | N |
| ATOM | 7634 | CA | ASP | D | 178 | 20.410 | 17.117 | −23.199 | 1.00 | 58.43 | C |
| ATOM | 7635 | C | ASP | D | 178 | 20.188 | 15.602 | −23.170 | 1.00 | 57.78 | C |
| ATOM | 7636 | O | ASP | D | 178 | 21.142 | 14.821 | −23.216 | 1.00 | 48.31 | O |
| ATOM | 7637 | CB | ASP | D | 178 | 20.597 | 17.530 | −24.662 | 1.00 | 56.35 | C |
| ATOM | 7638 | CG | ASP | D | 178 | 20.395 | 19.012 | −24.878 | 1.00 | 98.69 | C |
| ATOM | 7639 | OD1 | ASP | D | 178 | 20.173 | 19.735 | −23.879 | 1.00 | 105.79 | O |
| ATOM | 7640 | OD2 | ASP | D | 178 | 20.461 | 19.453 | −26.047 | 1.00 | 118.01 | O |
| ATOM | 7641 | N | PRO | D | 179 | 18.911 | 15.183 | −23.160 | 1.00 | 59.03 | N |
| ATOM | 7642 | CA | PRO | D | 179 | 18.595 | 13.754 | −23.098 | 1.00 | 55.25 | C |
| ATOM | 7643 | C | PRO | D | 179 | 19.170 | 13.067 | −24.332 | 1.00 | 49.95 | C |
| ATOM | 7644 | O | PRO | D | 179 | 19.646 | 11.929 | −24.286 | 1.00 | 39.90 | O |
| ATOM | 7645 | CB | PRO | D | 179 | 17.058 | 13.726 | −23.144 | 1.00 | 55.74 | C |
| ATOM | 7646 | CG | PRO | D | 179 | 16.608 | 15.162 | −23.004 | 1.00 | 53.23 | C |
| ATOM | 7647 | CD | PRO | D | 179 | 17.730 | 15.998 | −23.491 | 1.00 | 53.75 | C |
| ATOM | 7648 | N | GLN | D | 180 | 19.117 | 13.786 | −25.445 | 1.00 | 46.60 | N |
| ATOM | 7649 | CA | GLN | D | 180 | 19.661 | 13.295 | −26.692 | 1.00 | 53.46 | C |
| ATOM | 7650 | C | GLN | D | 180 | 21.134 | 12.993 | −26.495 | 1.00 | 44.53 | C |
| ATOM | 7651 | O | GLN | D | 180 | 21.580 | 11.876 | −26.744 | 1.00 | 39.60 | O |
| ATOM | 7652 | CB | GLN | D | 180 | 19.456 | 14.333 | −27.794 | 1.00 | 65.36 | C |
| ATOM | 7653 | CG | GLN | D | 180 | 17.988 | 14.706 | −28.028 | 1.00 | 76.49 | C |
| ATOM | 7654 | CD | GLN | D | 180 | 17.243 | 13.690 | −28.881 | 1.00 | 87.77 | C |
| ATOM | 7655 | OE1 | GLN | D | 180 | 17.837 | 13.001 | −29.715 | 1.00 | 86.58 | O |
| ATOM | 7656 | NE2 | GLN | D | 180 | 15.931 | 13.601 | −28.681 | 1.00 | 89.58 | N |
| ATOM | 7657 | N | ALA | D | 181 | 21.878 | 13.990 | −26.023 | 1.00 | 48.39 | N |
| ATOM | 7658 | CA | ALA | D | 181 | 23.277 | 13.798 | −25.644 | 1.00 | 40.56 | C |
| ATOM | 7659 | C | ALA | D | 181 | 23.419 | 12.624 | −24.682 | 1.00 | 40.69 | C |
| ATOM | 7660 | O | ALA | D | 181 | 24.271 | 11.754 | −24.862 | 1.00 | 29.47 | O |
| ATOM | 7661 | CB | ALA | D | 181 | 23.824 | 15.057 | −25.004 | 1.00 | 39.08 | C |
| ATOM | 7662 | N | LEU | D | 182 | 22.563 | 12.606 | −23.663 | 1.00 | 38.04 | N |
| ATOM | 7663 | CA | LEU | D | 182 | 22.621 | 11.592 | −22.617 | 1.00 | 34.04 | C |
| ATOM | 7664 | C | LEU | D | 182 | 22.414 | 10.157 | −23.122 | 1.00 | 31.64 | C |
| ATOM | 7665 | O | LEU | D | 182 | 23.108 | 9.237 | −22.698 | 1.00 | 32.96 | O |
| ATOM | 7666 | CB | LEU | D | 182 | 21.651 | 11.935 | −21.478 | 1.00 | 33.24 | C |
| ATOM | 7667 | CG | LEU | D | 182 | 22.020 | 13.161 | −20.627 | 1.00 | 32.58 | C |
| ATOM | 7668 | CD1 | LEU | D | 182 | 21.097 | 13.285 | −19.460 | 1.00 | 31.48 | C |
| ATOM | 7669 | CD2 | LEU | D | 182 | 23.449 | 13.069 | −20.129 | 1.00 | 35.88 | C |
| ATOM | 7670 | N | LYS | D | 183 | 21.480 | 9.960 | −24.037 | 1.00 | 35.19 | N |
| ATOM | 7671 | CA | LYS | D | 183 | 21.309 | 8.637 | −24.635 | 1.00 | 42.64 | C |
| ATOM | 7672 | C | LYS | D | 183 | 22.533 | 8.195 | −25.457 | 1.00 | 43.62 | C |
| ATOM | 7673 | O | LYS | D | 183 | 22.888 | 7.014 | −25.463 | 1.00 | 37.74 | O |
| ATOM | 7674 | CB | LYS | D | 183 | 20.007 | 8.575 | −25.434 | 1.00 | 42.72 | C |
| ATOM | 7675 | CG | LYS | D | 183 | 18.810 | 8.991 | −24.573 | 1.00 | 66.04 | C |
| ATOM | 7676 | CD | LYS | D | 183 | 17.460 | 8.652 | −25.186 | 1.00 | 75.45 | C |
| ATOM | 7677 | CE | LYS | D | 183 | 16.329 | 9.201 | −24.328 | 1.00 | 68.06 | C |
| ATOM | 7678 | NZ | LYS | D | 183 | 14.993 | 8.774 | −24.818 | 1.00 | 79.76 | N |
| ATOM | 7679 | N | CYS | D | 184 | 23.191 | 9.146 | −26.122 | 1.00 | 38.11 | N |
| ATOM | 7680 | CA | CYS | D | 184 | 24.459 | 8.871 | −26.801 | 1.00 | 36.49 | C |
| ATOM | 7681 | C | CYS | D | 184 | 25.565 | 8.479 | −25.814 | 1.00 | 35.00 | C |
| ATOM | 7682 | O | CYS | D | 184 | 26.476 | 7.722 | −26.158 | 1.00 | 42.57 | O |
| ATOM | 7683 | CB | CYS | D | 184 | 24.913 | 10.084 | −27.620 | 1.00 | 41.76 | C |
| ATOM | 7684 | SG | CYS | D | 184 | 26.088 | 9.685 | −28.938 | 1.00 | 77.84 | S |
| ATOM | 7685 | N | TYR | D | 185 | 25.497 | 9.000 | −24.594 | 1.00 | 25.67 | N |
| ATOM | 7686 | CA | TYR | D | 185 | 26.461 | 8.615 | −23.583 | 1.00 | 27.83 | C |
| ATOM | 7687 | C | TYR | D | 185 | 26.204 | 7.210 | −23.052 | 1.00 | 35.95 | C |
| ATOM | 7688 | O | TYR | D | 185 | 27.141 | 6.524 | −22.656 | 1.00 | 40.48 | O |
| ATOM | 7689 | CB | TYR | D | 185 | 26.529 | 9.631 | −22.441 | 1.00 | 28.32 | C |
| ATOM | 7690 | CG | TYR | D | 185 | 26.846 | 11.030 | −22.906 | 1.00 | 29.33 | C |
| ATOM | 7691 | CD1 | TYR | D | 185 | 27.589 | 11.240 | −24.060 | 1.00 | 21.87 | C |
| ATOM | 7692 | CD2 | TYR | D | 185 | 26.407 | 12.142 | −22.191 | 1.00 | 31.54 | C |
| ATOM | 7693 | CE1 | TYR | D | 185 | 27.877 | 12.508 | −24.504 | 1.00 | 17.57 | C |
| ATOM | 7694 | CE2 | TYR | D | 185 | 26.700 | 13.421 | −22.616 | 1.00 | 30.13 | C |
| ATOM | 7695 | CZ | TYR | D | 185 | 27.440 | 13.598 | −23.778 | 1.00 | 30.22 | C |
| ATOM | 7696 | OH | TYR | D | 185 | 27.740 | 14.869 | −24.220 | 1.00 | 31.93 | O |
| ATOM | 7697 | N | GLN | D | 186 | 24.948 | 6.765 | −23.055 | 1.00 | 39.80 | N |
| ATOM | 7698 | CA | GLN | D | 186 | 24.671 | 5.369 | −22.681 | 1.00 | 41.49 | C |
| ATOM | 7699 | C | GLN | D | 186 | 24.934 | 4.346 | −23.795 | 1.00 | 39.37 | C |
| ATOM | 7700 | O | GLN | D | 186 | 25.522 | 3.303 | −23.534 | 1.00 | 38.57 | O |
| ATOM | 7701 | CB | GLN | D | 186 | 23.291 | 5.168 | −22.026 | 1.00 | 34.03 | C |
| ATOM | 7702 | CG | GLN | D | 186 | 22.262 | 6.257 | −22.265 | 1.00 | 51.03 | C |
| ATOM | 7703 | CD | GLN | D | 186 | 20.969 | 6.025 | −21.471 | 1.00 | 61.24 | C |
| ATOM | 7704 | OE1 | GLN | D | 186 | 20.943 | 5.243 | −20.513 | 1.00 | 62.55 | O |
| ATOM | 7705 | NE2 | GLN | D | 186 | 19.896 | 6.709 | −21.869 | 1.00 | 50.15 | N |
| ATOM | 7706 | N | ASP | D | 187 | 24.526 | 4.645 | −25.028 | 1.00 | 38.37 | N |
| ATOM | 7707 | CA | ASP | D | 187 | 24.813 | 3.740 | −26.139 | 1.00 | 41.84 | C |
| ATOM | 7708 | C | ASP | D | 187 | 26.320 | 3.684 | −26.403 | 1.00 | 43.32 | C |
| ATOM | 7709 | O | ASP | D | 187 | 26.957 | 4.711 | −26.677 | 1.00 | 40.88 | O |
| ATOM | 7710 | CB | ASP | D | 187 | 24.055 | 4.155 | −27.410 | 1.00 | 37.79 | C |
| ATOM | 7711 | CG | ASP | D | 187 | 23.886 | 3.000 | −28.410 | 1.00 | 51.75 | C |
| ATOM | 7712 | OD1 | ASP | D | 187 | 24.559 | 1.948 | −28.267 | 1.00 | 51.05 | O |
| ATOM | 7713 | OD2 | ASP | D | 187 | 23.071 | 3.145 | −29.347 | 1.00 | 49.88 | O |
| ATOM | 7714 | N | PRO | D | 188 | 26.903 | 2.481 | −26.292 | 1.00 | 38.36 | N |
| ATOM | 7715 | CA | PRO | D | 188 | 28.322 | 2.295 | −26.595 | 1.00 | 43.95 | C |
| ATOM | 7716 | C | PRO | D | 188 | 28.478 | 2.295 | −28.100 | 1.00 | 46.22 | C |
| ATOM | 7717 | O | PRO | D | 188 | 29.540 | 2.638 | −28.626 | 1.00 | 51.98 | O |
| ATOM | 7718 | CB | PRO | D | 188 | 28.632 | 0.901 | −26.036 | 1.00 | 34.89 | C |
| ATOM | 7719 | CG | PRO | D | 188 | 27.449 | 0.515 | −25.218 | 1.00 | 43.85 | C |
| ATOM | 7720 | CD | PRO | D | 188 | 26.283 | 1.245 | −25.806 | 1.00 | 39.53 | C |
| ATOM | 7721 | N | GLY | D | 189 | 27.401 | 1.917 | −28.782 | 1.00 | 35.98 | N |
| ATOM | 7722 | CA | GLY | D | 189 | 27.369 | 1.893 | −30.230 | 1.00 | 29.99 | C |
| ATOM | 7723 | C | GLY | D | 189 | 27.192 | 3.271 | −30.826 | 1.00 | 33.06 | C |
| ATOM | 7724 | O | GLY | D | 189 | 27.220 | 3.438 | −32.041 | 1.00 | 42.62 | O |
| ATOM | 7725 | N | CYS | D | 190 | 26.998 | 4.269 | −29.975 | 1.00 | 36.26 | N |
| ATOM | 7726 | CA | CYS | D | 190 | 26.980 | 5.642 | −30.450 | 1.00 | 36.63 | C |
| ATOM | 7727 | C | CYS | D | 190 | 28.260 | 6.372 | −30.048 | 1.00 | 36.49 | C |
| ATOM | 7728 | O | CYS | D | 190 | 28.602 | 6.474 | −28.866 | 1.00 | 36.99 | O |
| ATOM | 7729 | CB | CYS | D | 190 | 25.754 | 6.390 | −29.943 | 1.00 | 25.70 | C |
| ATOM | 7730 | SG | CYS | D | 190 | 25.842 | 8.157 | −30.296 | 1.00 | 52.32 | S |
| ATOM | 7731 | N | CYS | D | 191 | 28.972 | 6.869 | −31.047 | 1.00 | 32.07 | N |
| ATOM | 7732 | CA | CYS | D | 191 | 30.217 | 7.571 | −30.805 | 1.00 | 33.08 | C |
| ATOM | 7733 | C | CYS | D | 191 | 30.170 | 8.942 | −31.430 | 1.00 | 31.14 | C |
| ATOM | 7734 | O | CYS | D | 191 | 31.041 | 9.312 | −32.211 | 1.00 | 33.71 | O |
| ATOM | 7735 | CB | CYS | D | 191 | 31.417 | 6.808 | −31.367 | 1.00 | 30.22 | C |
| ATOM | 7736 | SG | CYS | D | 191 | 32.981 | 7.535 | −30.803 | 1.00 | 43.00 | S |
| ATOM | 7737 | N | ASP | D | 192 | 29.137 | 9.689 | −31.082 | 1.00 | 26.62 | N |
| ATOM | 7738 | CA | ASP | D | 192 | 28.949 | 11.021 | −31.617 | 1.00 | 29.40 | C |
| ATOM | 7739 | C | ASP | D | 192 | 29.511 | 12.048 | −30.651 | 1.00 | 35.64 | C |
| ATOM | 7740 | O | ASP | D | 192 | 29.383 | 11.930 | −29.423 | 1.00 | 32.14 | O |
| ATOM | 7741 | CB | ASP | D | 192 | 27.466 | 11.265 | −31.903 | 1.00 | 39.75 | C |
| ATOM | 7742 | CG | ASP | D | 192 | 26.842 | 10.151 | −32.764 | 1.00 | 70.76 | C |
| ATOM | 7743 | OD1 | ASP | D | 192 | 27.600 | 9.374 | −33.398 | 1.00 | 61.18 | O |
| ATOM | 7744 | OD2 | ASP | D | 192 | 25.590 | 10.048 | −32.803 | 1.00 | 77.41 | O |
| ATOM | 7745 | N | PHE | D | 193 | 30.165 | 13.050 | −31.212 | 1.00 | 27.86 | N |
| ATOM | 7746 | CA | PHE | D | 193 | 30.846 | 14.032 | −30.389 | 1.00 | 34.78 | C |
| ATOM | 7747 | C | PHE | D | 193 | 29.871 | 15.165 | −30.119 | 1.00 | 34.95 | C |
| ATOM | 7748 | O | PHE | D | 193 | 30.040 | 16.284 | −30.623 | 1.00 | 29.04 | O |
| ATOM | 7749 | CB | PHE | D | 193 | 32.135 | 14.511 | −31.068 | 1.00 | 28.13 | C |
| ATOM | 7750 | CG | PHE | D | 193 | 33.109 | 15.159 | −30.138 | 1.00 | 22.65 | C |
| ATOM | 7751 | CD1 | PHE | D | 193 | 33.233 | 14.729 | −28.824 | 1.00 | 29.17 | C |
| ATOM | 7752 | CD2 | PHE | D | 193 | 33.921 | 16.194 | −30.579 | 1.00 | 21.58 | C |
| ATOM | 7753 | CE1 | PHE | D | 193 | 34.150 | 15.344 | −27.948 | 1.00 | 32.98 | C |
| ATOM | 7754 | CE2 | PHE | D | 193 | 34.834 | 16.806 | −29.722 | 1.00 | 28.32 | C |
| ATOM | 7755 | CZ | PHE | D | 193 | 34.955 | 16.384 | −28.404 | 1.00 | 19.52 | C |
| ATOM | 7756 | N | VAL | D | 194 | 28.837 | 14.831 | −29.337 | 1.00 | 32.39 | N |
| ATOM | 7757 | CA | VAL | D | 194 | 27.814 | 15.776 | −28.891 | 1.00 | 31.06 | C |
| ATOM | 7758 | C | VAL | D | 194 | 28.337 | 16.523 | −27.670 | 1.00 | 28.02 | C |
| ATOM | 7759 | O | VAL | D | 194 | 28.441 | 15.964 | −26.586 | 1.00 | 30.42 | O |
| ATOM | 7760 | CB | VAL | D | 194 | 26.505 | .15.054 | −28.486 | 1.00 | 35.21 | C |
| ATOM | 7761 | CG1 | VAL | D | 194 | 25.326 | 16.001 | −28.591 | 1.00 | 30.22 | C |
| ATOM | 7762 | CG2 | VAL | D | 194 | 26.279 | 13.809 | −29.338 | 1.00 | 33.68 | C |
| ATOM | 7763 | N | THR | D | 195 | 28.670 | 17.788 | −27.840 | 1.00 | 24.75 | N |
| ATOM | 7764 | CA | THR | D | 195 | 29.314 | 18.506 | −26.757 | 1.00 | 33.18 | C |
| ATOM | 7765 | C | THR | D | 195 | 28.457 | 19.680 | −26.336 | 1.00 | 28.23 | C |
| ATOM | 7766 | O | THR | D | 195 | 27.494 | 20.023 | −27.005 | 1.00 | 28.87 | O |
| ATOM | 7767 | CB | THR | D | 195 | 30.738 | 18.983 | −27.161 | 1.00 | 35.95 | C |
| ATOM | 7768 | OG1 | THR | D | 195 | 30.649 | 20.103 | −28.050 | 1.00 | 34.83 | O |
| ATOM | 7769 | CG2 | THR | D | 195 | 31.507 | 17.857 | −27.854 | 1.00 | 28.62 | C |
| ATOM | 7770 | N | ASN | D | 196 | 28.803 | 20.294 | −25.218 | 1.00 | 31.98 | N |
| ATOM | 7771 | CA | ASN | D | 196 | 28.109 | 21.497 | −24.798 | 1.00 | 31.91 | C |
| ATOM | 7772 | C | ASN | D | 196 | 28.638 | 22.714 | −25.577 | 1.00 | 32.87 | C |
| ATOM | 7773 | O | ASN | D | 196 | 29.750 | 22.672 | −26.123 | 1.00 | 29.48 | O |
| ATOM | 7774 | CB | ASN | D | 196 | 28.208 | 21.663 | −23.281 | 1.00 | 29.86 | C |
| ATOM | 7775 | CG | ASN | D | 196 | 29.638 | 21.826 | −22.791 | 1.00 | 26.56 | C |
| ATOM | 7776 | OD1 | ASN | D | 196 | 30.243 | 22.883 | −22.955 | 1.00 | 25.52 | O |
| ATOM | 7777 | ND2 | ASN | D | 196 | 30.164 | 20.797 | −22.151 | 1.00 | 26.89 | N |
| ATOM | 7778 | N | ARG | D | 197 | 27.839 | 23.776 | −25.664 | 1.00 | 26.82 | N |
| ATOM | 7779 | CA | ARG | D | 197 | 28.227 | 24.950 | −26.450 | 1.00 | 24.80 | C |
| ATOM | 7780 | C | ARG | D | 197 | 29.503 | 25.632 | −25.933 | 1.00 | 28.25 | C |
| ATOM | 7781 | O | ARG | D | 197 | 30.349 | 26.074 | −26.729 | 1.00 | 27.93 | O |
| ATOM | 7782 | CB | ARG | D | 197 | 27.081 | 25.966 | −26.562 | 1.00 | 23.60 | C |
| ATOM | 7783 | CG | ARG | D | 197 | 25.756 | 25.397 | −27.060 | 1.00 | 36.35 | C |
| ATOM | 7784 | CD | ARG | D | 197 | 24.745 | 26.516 | −27.305 | 1.00 | 47.96 | C |
| ATOM | 7785 | NE | ARG | D | 197 | 23.400 | 26.025 | −27.622 | 1.00 | 74.96 | N |
| ATOM | 7786 | CZ | ARG | D | 197 | 22.375 | 26.808 | −27.969 | 1.00 | 97.26 | C |
| ATOM | 7787 | NH1 | ARG | D | 197 | 22.529 | 28.127 | −28.053 | 1.00 | 89.57 | N |
| ATOM | 7788 | NH2 | ARG | D | 197 | 21.187 | 26.278 | −28.238 | 1.00 | 99.42 | N |
| ATOM | 7789 | N | ALA | D | 198 | 29.643 | 25.714 | −24.608 | 1.00 | 25.83 | N |
| ATOM | 7790 | CA | ALA | D | 198 | 30.849 | 26.276 | −23.992 | 1.00 | 27.91 | C |
| ATOM | 7791 | C | ALA | D | 198 | 32.099 | 25.597 | −24.537 | 1.00 | 29.61 | C |
| ATOM | 7792 | O | ALA | D | 198 | 32.997 | 26.254 | −25.060 | 1.00 | 27.15 | O |
| ATOM | 7793 | CB | ALA | D | 198 | 30.801 | 26.142 | −22.484 | 1.00 | 20.54 | C |
| ATOM | 7794 | N | TYR | D | 199 | 32.144 | 24.275 | −24.411 | 1.00 | 26.38. | N |
| ATOM | 7795 | CA | TYR | D | 199 | 33.279 | 23.506 | −24.870 | 1.00 | 21.80 | C |
| ATOM | 7796 | C | TYR | D | 199 | 33.487 | 23.659 | −26.370 | 1.00 | 27.77 | C |
| ATOM | 7797 | O | TYR | D | 199 | 34.593 | 23.934 | −26.829 | 1.00 | 25.65 | O |
| ATOM | 7798 | CB | TYR | D | 199 | 33.106 | 22.031 | −24.521 | 1.00 | 23.55 | C |
| ATOM | 7799 | CG | TYR | D | 199 | 34.192 | 21.178 | −25.132 | 1.00 | 29.28 | C |
| ATOM | 7800 | CD1 | TYR | D | 199 | 35.413 | 20.986 | −24.481 | 1.00 | 26.04 | C |
| ATOM | 7801 | CD2 | TYR | D | 199 | 34.014 | 20.595 | −26.379 | 1.00 | 28.10 | C |
| ATOM | 7802 | CE1 | TYR | D | 199 | 36.401 | 20.214 | −25.045 | 1.00 | 22.24 | C |
| ATOM | 7803 | CE2 | TYR | D | 199 | 34.999 | 19.827 | −26.950 | 1.00 | 30.43 | C |
| ATOM | 7804 | CZ | TYR | D | 199 | 36.189 | 19.638 | −26.283 | 1.00 | 23.00 | C |
| ATOM | 7805 | OH | TYR | D | 199 | 37.154 | 18.868 | −26.879 | 1.00 | 21.93 | O |
| ATOM | 7806 | N | ALA | D | 200 | 32.415 | 23.476 | −27.130 | 1.00 | 31.39 | N |
| ATOM | 7807 | CA | ALA | D | 200 | 32.507 | 23.531 | −28.573 | 1.00 | 24.70 | C |
| ATOM | 7808 | C | ALA | D | 200 | 33.201 | 24.820 | −29.026 | 1.00 | 28.48 | C |
| ATOM | 7809 | O | ALA | D | 200 | 34.113 | 24.779 | −29.845 | 1.00 | 32.06 | O |
| ATOM | 7810 | CB | ALA | D | 200 | 31.134 | 23.399 | −29.197 | 1.00 | 24.71 | C |
| ATOM | 7811 | N | ILE | D | 201 | 32.788 | 25.961 | −28.489 | 1.00 | 23.38 | N |
| ATOM | 7812 | CA | ILE | D | 201 | 33.410 | 27.224 | −28.873 | 1.00 | 23.66 | C |
| ATOM | 7813 | C | ILE | D | 201 | 34.849 | 27.361 | −28.378 | 1.00 | 30.86 | C |
| ATOM | 7814 | O | ILE | D | 201 | 35.738 | 27.769 | −29.116 | 1.00 | 32.71 | O |
| ATOM | 7815 | CB | ILE | D | 201 | 32.605 | 28.420 | −28.367 | 1.00 | 31.04 | C |
| ATOM | 7816 | CG1 | ILE | D | 201 | 31.334 | 28.580 | −29.209 | 1.00 | 30.75 | C |
| ATOM | 7817 | CG2 | ILE | D | 201 | 33.468 | 29.687 | −28.377 | 1.00 | 18.04 | C |
| ATOM | 7818 | CD1 | ILE | D | 201 | 30.307 | 29.492 | −28.582 | 1.00 | 30.83 | C |
| ATOM | 7819 | N | ALA | D | 202 | 35.081 | 27.015 | −27.121 | 1.00 | 33.20 | N |
| ATOM | 7820 | CA | ALA | D | 202 | 36.397 | 27.184 | −26.522 | 1.00 | 31.65 | C |
| ATOM | 7821 | C | ALA | D | 202 | 37.460 | 26.281 | −27.152 | 1.00 | 30.14 | C |
| ATOM | 7822 | O | ALA | D | 202 | 38.543 | 26.745 | −27.506 | 1.00 | 27.71 | O |
| ATOM | 7823 | CB | ALA | D | 202 | 36.326 | 26.975 | −25.006 | 1.00 | 25.03 | C |
| ATOM | 7824 | N | SER | D | 203 | 37.149 | 25.000 | −27.295 | 1.00 | 25.50 | N |
| ATOM | 7825 | CA | SER | D | 203 | 38.127 | 24.067 | −27.824 | 1.00 | 30.78 | C |
| ATOM | 7826 | C | SER | D | 203 | 38.459 | 24.391 | −29.267 | 1.00 | 32.08 | C |
| ATOM | 7827 | O | SER | D | 203 | 39.520 | 24.011 | −29.766 | 1.00 | 28.24 | O |
| ATOM | 7828 | CB | SER | D | 203 | 37.637 | 22.625 | −27.706 | 1.00 | 31.60 | C |
| ATOM | 7829 | OG | SER | D | 203 | 36.694 | 22.337 | −28.706 | 1.00 | 29.65 | O |
| ATOM | 7830 | N | SER | D | 204 | 37.552 | 25.107 | −29.922 | 1.00 | 25.47 | N |
| ATOM | 7831 | CA | SER | D | 204 | 37.737 | 25.498 | −31.308 | 1.00 | 29.15 | C |
| ATOM | 7832 | C | SER | D | 204 | 38.580 | 26.763 | −31.444 | 1.00 | 27.51 | C |
| ATOM | 7833 | O | SER | D | 204 | 39.483 | 26.837 | −32.270 | 1.00 | 29.51 | O |
| ATOM | 7834 | CB | SER | D | 204 | 36.380 | 25.681 | −31.973 | 1.00 | 28.56 | C |
| ATOM | 7835 | OG | SER | D | 204 | 35.663 | 24.463 | −31.915 | 1.00 | 36.73 | O |
| ATOM | 7836 | N | ILE | D | 205 | 38.265 | 27.762 | −30.641 | 1.00 | 28.55 | N |
| ATOM | 7837 | CA | ILE | D | 205 | 39.091 | 28.950 | −30.549 | 1.00 | 29.74 | C |
| ATOM | 7838 | C | ILE | D | 205 | 40.528 | 28.557 | −30.204 | 1.00 | 33.12 | C |
| ATOM | 7839 | O | ILE | D | 205 | 41.489 | 29.091 | −30.757 | 1.00 | 32.89 | O |
| ATOM | 7840 | CB | ILE | D | 205 | 38.531 | 29.902 | −29.476 | 1.00 | 33.98 | C |
| ATOM | 7841 | CG1 | ILE | D | 205 | 37.400 | 30.736 | −30.071 | 1.00 | 37.99 | C |
| ATOM | 7842 | CG2 | ILE | D | 205 | 39.610 | 30.813 | −28.918 | 1.00 | 29.96 | C |
| ATOM | 7843 | CD1 | ILE | D | 205 | 36.569 | 31.463 | −29.036 | 1.00 | 30.28 | C |
| ATOM | 7844 | N | ILE | D | 206 | 40.660 | 27.587 | −29.310 | 1.00 | 31.42 | N |
| ATOM | 7845 | CA | ILE | D | 206 | 41.956 | 27.220 | −28.737 | 1.00 | 31.08 | C |
| ATOM | 7846 | C | ILE | D | 206 | 42.779 | 26.191 | −29.524 | 1.00 | 30.42 | C |
| ATOM | 7847 | O | ILE | D | 206 | 44.007 | 26.238 | −29.492 | 1.00 | 33.32 | O |
| ATOM | 7848 | CB | ILE | D | 206 | 41.790 | 26.776 | −27.256 | 1.00 | 30.45 | C |
| ATOM | 7849 | CG1 | ILE | D | 206 | 41.945 | 27.991 | −26.339 | 1.00 | 30.34 | C |
| ATOM | 7850 | CG2 | ILE | D | 206 | 42.775 | 25.677 | −26.871 | 1.00 | 27.60 | C |
| ATOM | 7851 | CD1 | ILE | D | 206 | 41.062 | 27.944 | −25.123 | 1.00 | 33.24 | C |
| ATOM | 7852 | N | SER | D | 207 | 42.124 | 25.263 | −30.216 | 1.00 | 26.76 | N |
| ATOM | 7853 | CA | SER | D | 207 | 42.850 | 24.275 | −31.011 | 1.00 | 25.10 | C |
| ATOM | 7854 | C | SER | D | 207 | 43.092 | 24.774 | −32.421 | 1.00 | 25.42 | C |
| ATOM | 7855 | O | SER | D | 207 | 44.051 | 24.369 | −33.056 | 1.00 | 32.29 | O |
| ATOM | 7856 | CB | SER | D | 207 | 42.093 | 22.943 | −31.087 | 1.00 | 28.19 | C |
| ATOM | 7857 | OG | SER | D | 207 | 42.052 | 22.281 | −29.835 | 1.00 | 37.11 | O |
| ATOM | 7858 | N | PHE | D | 208 | 42.232 | 25.661 | −32.906 | 1.00 | 25.72 | N |
| ATOM | 7859 | CA | PHE | D | 208 | 42.198 | 25.934 | −34.334 | 1.00 | 26.50 | C |
| ATOM | 7860 | C | PHE | D | 208 | 42.313 | 27.403 | −34.734 | 1.00 | 28.53 | C |
| ATOM | 7861 | O | PHE | D | 208 | 43.270 | 27.781 | −35.418 | 1.00 | 35.92 | O |
| ATOM | 7862 | CB | PHE | D | 208 | 40.955 | 25.294 | −34.987 | 1.00 | 26.14 | C |
| ATOM | 7863 | CG | PHE | D | 208 | 40.919 | 25.458 | −36.477 | 1.00 | 25.37 | C |
| ATOM | 7864 | CD1 | PHE | D | 208 | 41.694 | 24.653 | −37.296 | 1.00 | 28.28 | C |
| ATOM | 7865 | CD2 | PHE | D | 208 | 40.143 | 26.444 | −37.056 | 1.00 | 24.90 | C |
| ATOM | 7866 | CE1 | PHE | D | 208 | 41.688 | 24.825 | −38.660 | 1.00 | 21.59 | C |
| ATOM | 7867 | CE2 | PHE | D | 208 | 40.126 | 26.605 | −38.411 | 1.00 | 22.80 | C |
| ATOM | 7868 | CZ | PHE | D | 208 | 40.902 | 25.798 | −39.215 | 1.00 | 21.99 | C |
| ATOM | 7869 | N | TYR | D | 209 | 41.340 | 28.216 | −34.328 | 1.00 | 29.97 | N |
| ATOM | 7870 | CA | TYR | D | 209 | 41.245 | 29.609 | −34.789 | 1.00 | 36.31 | C |
| ATOM | 7871 | C | TYR | D | 209 | 42.411 | 30.513 | −34.368 | 1.00 | 36.72 | C |
| ATOM | 7872 | O | TYR | D | 209 | 42.888 | 31.320 | −35.166 | 1.00 | 35.91 | O |
| ATOM | 7873 | CB | TYR | D | 209 | 39.898 | 30.248 | −34.397 | 1.00 | 29.81 | C |
| ATOM | 7874 | CG | TYR | D | 209 | 38.723 | 29.731 | −35.199 | 1.00 | 33.01 | C |
| ATOM | 7875 | CD1 | TYR | D | 209 | 37.760 | 28.928 | −34.611 | 1.00 | 35.42 | C |
| ATOM | 7876 | CD2 | TYR | D | 209 | 38.586 | 30.029 | −36.549 | 1.00 | 38.73 | C |
| ATOM | 7877 | CE1 | TYR | D | 209 | 36.681 | 28.447 | −35.336 | 1.00 | 30.25 | C |
| ATOM | 7878 | CE2 | TYR | D | 209 | 37.519 | 29.547 | −37.284 | 1.00 | 35.05 | C |
| ATOM | 7879 | CZ | TYR | D | 209 | 36.569 | 28.752 | −36.668 | 1.00 | 42.05 | C |
| ATOM | 7880 | OH | TYR | D | 209 | 35.500 | 28.255 | −37.382 | 1.00 | 45.10 | O |
| ATOM | 7881 | N | ILE | D | 210 | 42.876 | 30.397 | −33.130 | 1.00 | 32.26 | N |
| ATOM | 7882 | CA | ILE | D | 210 | 44.008 | 31.224 | −32.727 | 1.00 | 33.16 | C |
| ATOM | 7883 | C | ILE | D | 210 | 45.298 | 30.819 | −33.437 | 1.00 | 27.65 | C |
| ATOM | 7884 | O | ILE | D | 210 | 45.913 | 31.648 | −34.102 | 1.00 | 29.79 | O |
| ATOM | 7885 | CB | ILE | D | 210 | 44.182 | 31.302 | −31.197 | 1.00 | 30.55 | C |
| ATOM | 7886 | CG1 | ILE | D | 210 | 43.106 | 32.208 | −30.606 | 1.00 | 29.56 | C |
| ATOM | 7887 | CG2 | ILE | D | 210 | 45.552 | 31.849 | −30.829 | 1.00 | 31.81 | C |
| ATOM | 7888 | CD1 | ILE | D | 210 | 42.862 | 31.973 | −29.141 | 1.00 | 26.35 | C |
| ATOM | 7889 | N | PRO | D | 211 | 45.703 | 29.545 | −33.316 | 1.00 | 30.45 | N |
| ATOM | 7890 | CA | PRO | D | 211 | 46.911 | 29.112 | −34.041 | 1.00 | 29.58 | C |
| ATOM | 7891 | C | PRO | D | 211 | 46.801 | 29.474 | −35.518 | 1.00 | 34.65 | C |
| ATOM | 7892 | O | PRO | D | 211 | 47.792 | 29.860 | −36.124 | 1.00 | 39.89 | O |
| ATOM | 7893 | CB | PRO | D | 211 | 46.905 | 27.589 | −33.882 | 1.00 | 26.82 | C |
| ATOM | 7894 | CG | PRO | D | 211 | 46.063 | 27.323 | −32.687 | 1.00 | 32.49 | C |
| ATOM | 7895 | CD | PRO | D | 211 | 45.060 | 28.445 | −32.573 | 1.00 | 25.96 | C |
| ATOM | 7896 | N | LEU | D | 212 | 45.602 | 29.362 | −36.086 | 1.00 | 30.59 | N |
| ATOM | 7897 | CA | LEU | D | 212 | 45.375 | 29.751 | −37.479 | 1.00 | 32.12 | C |
| ATOM | 7898 | C | LEU | D | 212 | 45.639 | 31.236 | −37.745 | 1.00 | 34.58 | C |
| ATOM | 7899 | O | LEU | D | 212 | 46.424 | 31.581 | −38.629 | 1.00 | 38.10 | O |
| ATOM | 7900 | CB | LEU | D | 212 | 43.959 | 29.391 | −37.922 | 1.00 | 29.03 | C |
| ATOM | 7901 | CG | LEU | D | 212 | 43.732 | 29.483 | −39.431 | 1.00 | 27.22 | C |
| ATOM | 7902 | CD1 | LEU | D | 212 | 44.430 | 28.330 | −40.159 | 1.00 | 23.64 | C |
| ATOM | 7903 | CD2 | LEU | D | 212 | 42.261 | 29.493 | −39.731 | 1.00 | 25.17 | C |
| ATOM | 7904 | N | LEU | D | 213 | 44.978 | 32.104 | −36.980 | 1.00 | 34.16 | N |
| ATOM | 7905 | CA | LEU | D | 213 | 45.169 | 33.546 | −37.097 | 1.00 | 31.13 | C |
| ATOM | 7906 | C | LEU | D | 213 | 46.627 | 33.941 | −36.970 | 1.00 | 33.86 | C |
| ATOM | 7907 | O | LEU | D | 213 | 47.092 | 34.856 | −37.657 | 1.00 | 32.43 | O |
| ATOM | 7908 | CB | LEU | D | 213 | 44.340 | 34.284 | −36.059 | 1.00 | 33.64 | C |
| ATOM | 7909 | CG | LEU | D | 213 | 42.833 | 34.153 | −36.294 | 1.00 | 48.98 | C |
| ATOM | 7910 | CD1 | LEU | D | 213 | 42.050 | 34.651 | −35.095 | 1.00 | 42.89 | C |
| ATOM | 7911 | CD2 | LEU | D | 213 | 42.415 | 34.887 | −37.556 | 1.00 | 38.22 | C |
| ATOM | 7912 | N | ILE | D | 214 | 47.351 | 33.252 | −36.093 | 1.00 | 32.53 | N |
| ATOM | 7913 | CA | ILE | D | 214 | 48.782 | 33.504 | −35.926 | 1.00 | 36.72 | C |
| ATOM | 7914 | C | ILE | D | 214 | 49.548 | 33.077 | −37.169 | 1.00 | 40.01 | C |
| ATOM | 7915 | O | ILE | D | 214 | 50.307 | 33.857 | −37.756 | 1.00 | 41.52 | O |
| ATOM | 7916 | CB | ILE | D | 214 | 49.361 | 32.739 | −34.727 | 1.00 | 40.28 | C |
| ATOM | 7917 | CG1 | ILE | D | 214 | 48.861 | 33.348 | −33.423 | 1.00 | 29.41 | C |
| ATOM | 7918 | CG2 | ILE | D | 214 | 50.900 | 32.727 | −34.777 | 1.00 | 32.13 | C |
| ATOM | 7919 | CD1 | ILE | D | 214 | 49.267 | 32.552 | −32.239 | 1.00 | 31.92 | C |
| ATOM | 7920 | N | MET | D | 215 | 49.336 | 31.831 | −37.571 | 1.00 | 33.11 | N |
| ATOM | 7921 | CA | MET | D | 215 | 50.056 | 31.276 | −38.705 | 1.00 | 38.45 | C |
| ATOM | 7922 | C | MET | D | 215 | 49.898 | 32.133 | −39.957 | 1.00 | 40.90 | C |
| ATOM | 7923 | O | MET | D | 215 | 50.846 | 32.301 | −40.718 | 1.00 | 38.56 | O |
| ATOM | 7924 | CB | MET | D | 215 | 49.608 | 29.843 | −38.981 | 1.00 | 34.46 | C |
| ATOM | 7925 | CG | MET | D | 215 | 50.381 | 29.181 | −40.107 | 1.00 | 38.45 | C |
| ATOM | 7926 | SD | MET | D | 215 | 49.449 | 27.829 | −40.843 | 1.00 | 54.48 | S |
| ATOM | 7927 | CE | MET | D | 215 | 48.159 | 28.764 | −41.652 | 1.00 | 36.57 | C |
| ATOM | 7928 | N | ILE | D | 216 | 48.697 | 32.669 | −40.165 | 1.00 | 39.34 | N |
| ATOM | 7929 | CA | ILE | D | 216 | 48.431 | 33.501 | −41.332 | 1.00 | 37.46 | C |
| ATOM | 7930 | C | ILE | D | 216 | 49.120 | 34.847 | −41.221 | 1.00 | 44.19 | C |
| ATOM | 7931 | O | ILE | D | 216 | 49.873 | 35.233 | −42.112 | 1.00 | 47.97 | O |
| ATOM | 7932 | CB | ILE | D | 216 | 46.929 | 33.718 | −41.562 | 1.00 | 47.82 | C |
| ATOM | 7933 | CG1 | ILE | D | 216 | 46.361 | 32.557 | −42.384 | 1.00 | 41.64 | C |
| ATOM | 7934 | CG2 | ILE | D | 216 | 46.690 | 35.053 | −42.284 | 1.00 | 30.90 | C |
| ATOM | 7935 | CD1 | ILE | D | 216 | 44.917 | 32.252 | −42.080 | 1.00 | 38.28 | C |
| ATOM | 7936 | N | PHE | D | 217 | 48.866 | 35.563 | −40.130 | 1.00 | 37.99 | N |
| ATOM | 7937 | CA | PHE | D | 217 | 49.508 | 36.848 | −39.944 | 1.00 | 35.15 | C |
| ATOM | 7938 | C | PHE | D | 217 | 51.023 | 36.718 | −40.101 | 1.00 | 44.89 | C |
| ATOM | 7939 | O | PHE | D | 217 | 51.668 | 37.596 | −40.662 | 1.00 | 46.68 | O |
| ATOM | 7940 | CB | PHE | D | 217 | 49.167 | 37.445 | −38.584 | 1.00 | 46.62 | C |
| ATOM | 7941 | CG | PHE | D | 217 | 49.924 | 38.702 | −38.283 | 1.00 | 59.86 | C |
| ATOM | 7942 | CD1 | PHE | D | 217 | 49.641 | 39.873 | −38.964 | 1.00 | 63.34 | C |
| ATOM | 7943 | CD2 | PHE | D | 217 | 50.938 | 38.708 | −37.332 | 1.00 | 73.98 | C |
| ATOM | 7944 | CE1 | PHE | D | 217 | 50.346 | 41.033 | −38.699 | 1.00 | 80.21 | C |
| ATOM | 7945 | CE2 | PHE | D | 217 | 51.648 | 39.869 | −37.056 | 1.00 | 72.56 | C |
| ATOM | 7946 | CZ | PHE | D | 217 | 51.352 | 41.033 | −37.742 | 1.00 | 77.03 | C |
| ATOM | 7947 | N | VAL | D | 218 | 51.590 | 35.615 | −39.622 | 1.00 | 45.05 | N |
| ATOM | 7948 | CA | VAL | D | 218 | 53.034 | 35.415 | −39.716 | 1.00 | 43.64 | C |
| ATOM | 7949 | C | VAL | D | 218 | 53.473 | 35.039 | −41.132 | 1.00 | 41.05 | C |
| ATOM | 7950 | O | VAL | D | 218 | 54.420 | 35.609 | −41.668 | 1.00 | 44.65 | O |
| ATOM | 7951 | CB | VAL | D | 218 | 53.548 | 34.383 | −38.678 | 1.00 | 43.06 | C |
| ATOM | 7952 | CG1 | VAL | D | 218 | 54.987 | 33.954 | −38.995 | 1.00 | 34.70 | C |
| ATOM | 7953 | CG2 | VAL | D | 218 | 53.463 | 34.966 | −37.278 | 1.00 | 28.90 | C |
| ATOM | 7954 | N | ALA | D | 219 | 52.782 | 34.083 | −41.739 | 1.00 | 42.00 | N |
| ATOM | 7955 | CA | ALA | D | 219 | 53.079 | 33.688 | −43.111 | 1.00 | 39.49 | C |
| ATOM | 7956 | C | ALA | D | 219 | 52.995 | 34.869 | −44.074 | 1.00 | 42.38 | C |
| ATOM | 7957 | O | ALA | D | 219 | 53.753 | 34.944 | −45.035 | 1.00 | 44.49 | O |
| ATOM | 7958 | CB | ALA | D | 219 | 52.143 | 32.577 | −43.559 | 1.00 | 34.15 | C |
| ATOM | 7959 | N | LEU | D | 220 | 52.067 | 35.788 | −43.826 | 1.00 | 41.73 | N |
| ATOM | 7960 | CA | LEU | D | 220 | 51.956 | 36.974 | −44.668 | 1.00 | 43.00 | C |
| ATOM | 7961 | C | LEU | D | 220 | 53.175 | 37.894 | −44.534 | 1.00 | 46.18 | C |
| ATOM | 7962 | O | LEU | D | 220 | 53.646 | 38.449 | −45.520 | 1.00 | 45.02 | O |
| ATOM | 7963 | CB | LEU | D | 220 | 50.650 | 37.726 | −44.399 | 1.00 | 38.59 | C |
| ATOM | 7964 | CG | LEU | D | 220 | 49.383 | 37.034 | −44.936 | 1.00 | 54.14 | C |
| ATOM | 7965 | CD1 | LEU | D | 220 | 48.220 | 38.012 | −45.076 | 1.00 | 38.94 | C |
| ATOM | 7966 | CD2 | LEU | D | 220 | 49.630 | 36.311 | −46.268 | 1.00 | 26.68 | C |
| ATOM | 7967 | N | ARG | D | 221 | 53.698 | 38.041 | −43.326 | 1.00 | 47.95 | N |
| ATOM | 7968 | CA | ARG | D | 221 | 54.930 | 38.791 | −43.149 | 1.00 | 49.68 | C |
| ATOM | 7969 | C | ARG | D | 221 | 56.055 | 38.110 | −43.924 | 1.00 | 53.59 | C |
| ATOM | 7970 | O | ARG | D | 221 | 56.770 | 38.757 | −44.687 | 1.00 | 55.11 | O |
| ATOM | 7971 | CB | ARG | D | 221 | 55.297 | 38.897 | −41.666 | 1.00 | 43.56 | C |
| ATOM | 7972 | CG | ARG | D | 221 | 54.333 | 39.707 | −40.838 | 1.00 | 43.49 | C |
| ATOM | 7973 | CD | ARG | D | 221 | 54.194 | 41.121 | −41.359 | 1.00 | 59.36 | C |
| ATOM | 7974 | NE | ARG | D | 221 | 53.049 | 41.787 | −40.745 | 1.00 | 78.75 | N |
| ATOM | 7975 | CZ | ARG | D | 221 | 52.477 | 42.887 | −41.224 | 1.00 | 94.94 | C |
| ATOM | 7976 | NH1 | ARG | D | 221 | 52.948 | 43.447 | −42.331 | 1.00 | 100.40 | N |
| ATOM | 7977 | NH2 | ARG | D | 221 | 51.433 | 43.424 | −40.599 | 1.00 | 87.61 | N |
| ATOM | 7978 | N | VAL | D | 222 | 56.207 | 36.802 | −43.724 | 1.00 | 48.44 | N |
| ATOM | 7979 | CA | VAL | D | 222 | 57.272 | 36.049 | −44.383 | 1.00 | 45.75 | C |
| ATOM | 7980 | C | VAL | D | 222 | 57.215 | 36.234 | −45.896 | 1.00 | 50.10 | C |
| ATOM | 7981 | O | VAL | D | 222 | 58.242 | 36.416 | −46.541 | 1.00 | 57.15 | O |
| ATOM | 7982 | CB | VAL | D | 222 | 57.218 | 34.549 | −44.025 | 1.00 | 33.45 | C |
| ATOM | 7983 | CG1 | VAL | D | 222 | 58.177 | 33.739 | −44.875 | 1.00 | 27.41 | C |
| ATOM | 7984 | CG2 | VAL | D | 222 | 57.530 | 34.359 | −42.562 | 1.00 | 42.61 | C |
| ATOM | 7985 | N | TYR | D | 223 | 56.009 | 36.192 | −46.451 | 1.00 | 43.52 | N |
| ATOM | 7986 | CA | TYR | D | 223 | 55.785 | 36.502 | −47.860 | 1.00 | 53.84 | C |
| ATOM | 7987 | C | TYR | D | 223 | 56.295 | 37.896 | −48.250 | 1.00 | 59.98 | C |
| ATOM | 7988 | O | TYR | D | 223 | 57.036 | 38.035 | −49.210 | 1.00 | 54.81 | O |
| ATOM | 7989 | CB | TYR | D | 223 | 54.297 | 36.382 | −48.193 | 1.00 | 57.51 | C |
| ATOM | 7990 | CG | TYR | D | 223 | 53.945 | 36.673 | −49.639 | 1.00 | 76.86 | C |
| ATOM | 7991 | CD1 | TYR | D | 223 | 54.029 | 35.676 | −50.605 | 1.00 | 76.17 | C |
| ATOM | 7992 | CD2 | TYR | D | 223 | 53.511 | 37.939 | −50.035 | 1.00 | 76.34 | C |
| ATOM | 7993 | CE1 | TYR | D | 223 | 53.702 | 35.928 | −51.927 | 1.00 | 80.29 | C |
| ATOM | 7994 | CE2 | TYR | D | 223 | 53.185 | 38.204 | −51.356 | 1.00 | 75.80 | C |
| ATOM | 7995 | CZ | TYR | D | 223 | 53.281 | 37.191 | −52.299 | 1.00 | 93.37 | C |
| ATOM | 7996 | OH | TYR | D | 223 | 52.956 | 37.439 | −53.616 | 1.00 | 100.96 | O |
| ATOM | 7997 | N | ARG | D | 224 | 55.884 | 38.936 | −47.536 | 1.00 | 64.33 | N |
| ATOM | 7998 | CA | ARG | D | 224 | 56.396 | 40.261 | −47.850 | 1.00 | 64.63 | C |
| ATOM | 7999 | C | ARG | D | 224 | 57.915 | 40.232 | −47.801 | 1.00 | 70.13 | C |
| ATOM | 8000 | O | ARG | D | 224 | 58.568 | 40.663 | −48.744 | 1.00 | 68.16 | O |
| ATOM | 8001 | CB | ARG | D | 224 | 55.858 | 41.321 | −46.888 | 1.00 | 75.28 | C |
| ATOM | 8002 | CG | ARG | D | 224 | 54.339 | 41.428 | −46.869 | 1.00 | 79.04 | C |
| ATOM | 8003 | CD | ARG | D | 224 | 53.768 | 41.600 | −48.272 | 1.00 | 88.82 | C |
| ATOM | 8004 | NE | ARG | D | 224 | 52.306 | 41.619 | −48.258 | 1.00 | 89.80 | N |
| ATOM | 8005 | CZ | ARG | D | 224 | 51.545 | 41.992 | −49.285 | 1.00 | 90.40 | C |
| ATOM | 8006 | NH1 | ARG | D | 224 | 52.097 | 42.390 | −50.423 | 1.00 | 66.76 | N |
| ATOM | 8007 | NH2 | ARG | D | 224 | 50.225 | 41.971 | −49.172 | 1.00 | 104.25 | N |
| ATOM | 8008 | N | GLU | D | 225 | 58.477 | 39.700 | −46.715 | 1.00 | 65.17 | N |
| ATOM | 8009 | CA | GLU | D | 225 | 59.930 | 39.699 | −46.560 | 1.00 | 62.15 | C |
| ATOM | 8010 | C | GLU | D | 225 | 60.629 | 38.995 | −47.715 | 1.00 | 68.11 | C |
| ATOM | 8011 | O | GLU | D | 225 | 61.614 | 39.498 | −48.239 | 1.00 | 79.92 | O |
| ATOM | 8012 | CB | GLU | D | 225 | 60.358 | 39.082 | −45.229 | 1.00 | 57.07 | C |
| ATOM | 8013 | CG | GLU | D | 225 | 60.338 | 40.061 | −44.059 | 1.00 | 79.52 | C |
| ATOM | 8014 | CD | GLU | D | 225 | 61.448 | 41.095 | −44.137 | 1.00 | 86.64 | C |
| ATOM | 8015 | OE1 | GLU | D | 225 | 62.074 | 41.222 | −45.212 | 1.00 | 90.05 | O |
| ATOM | 8016 | OE2 | GLU | D | 225 | 61.698 | 41.780 | −43.120 | 1.00 | 86.69 | O |
| ATOM | 8017 | N | ALA | D | 226 | 60.117 | 37.834 | −48.109 | 1.00 | 63.51 | N |
| ATOM | 8018 | CA | ALA | D | 226 | 60.671 | 37.097 | −49.237 | 1.00 | 64.13 | C |
| ATOM | 8019 | C | ALA | D | 226 | 60.499 | 37.887 | −50.528 | 1.00 | 79.11 | C |
| ATOM | 8020 | O | ALA | D | 226 | 61.297 | 37.761 | −51.455 | 1.00 | 86.09 | O |
| ATOM | 8021 | CB | ALA | D | 226 | 60.011 | 35.736 | −49.360 | 1.00 | 54.87 | C |
| ATOM | 8022 | N | LYS | D | 227 | 59.457 | 38.711 | −50.580 | 1.00 | 76.98 | N |
| ATOM | 8023 | CA | LYS | D | 227 | 59.164 | 39.513 | −51.766 | 1.00 | 77.20 | C |
| ATOM | 8024 | C | LYS | D | 227 | 60.074 | 40.732 | −51.889 | 1.00 | 77.36 | C |
| ATOM | 8025 | O | LYS | D | 227 | 60.899 | 40.807 | −52.788 | 1.00 | 74.94 | O |
| ATOM | 8026 | CB | LYS | D | 227 | 57.703 | 39.964 | −51.754 | 1.00 | 87.99 | C |
| ATOM | 8027 | CG | LYS | D | 227 | 57.314 | 40.853 | −52.920 | 1.00 | 89.57 | C |
| ATOM | 8028 | CD | LYS | D | 227 | 56.103 | 41.712 | −52.573 | 1.00 | 90.25 | C |
| ATOM | 8029 | CE | LYS | D | 227 | 55.693 | 42.584 | −53.751 | 1.00 | 118.28 | C |
| ATOM | 8030 | NZ | LYS | D | 227 | 56.826 | 43.411 | −54.266 | 1.00 | 118.43 | N |
| ATOM | 8031 | N | GLU | D | 228 | 59.915 | 41.709 | −51.009 | 1.00 | 92.63 | N |
| ATOM | 8032 | CA | GLU | D | 228 | 60.721 | 42.915 | −51.124 | 1.00 | 85.87 | C |
| ATOM | 8033 | C | GLU | D | 228 | 62.197 | 42.536 | −51.171 | 1.00 | 88.02 | C |
| ATOM | 8034 | O | GLU | D | 228 | 63.054 | 43.359 | −51.493 | 1.00 | 96.21 | O |
| ATOM | 8035 | CB | GLU | D | 228 | 60.423 | 43.883 | −49.977 | 1.00 | 96.56 | C |
| ATOM | 8036 | CG | GLU | D | 228 | 58.941 | 44.191 | −49.778 | 1.00 | 100.69 | C |
| ATOM | 8037 | CD | GLU | D | 228 | 58.201 | 44.404 | −51.090 | 1.00 | 120.16 | C |
| ATOM | 8038 | OE1 | GLU | D | 228 | 58.859 | 44.683 | −52.120 | 1.00 | 116.52 | O |
| ATOM | 8039 | OE2 | GLU | D | 228 | 56.955 | 44.292 | −51.089 | 1.00 | 134.38 | O |
| ATOM | 8040 | N | GLN | D | 229 | 62.471 | 41.270 | −50.864 | 1.00 | 81.70 | N |
| ATOM | 8041 | CA | GLN | D | 229 | 63.820 | 40.733 | −50.851 | 1.00 | 85.83 | C |
| ATOM | 8042 | C | GLN | D | 229 | 64.268 | 40.441 | −52.268 | 1.00 | 93.42 | C |
| ATOM | 8043 | O | GLN | D | 229 | 65.382 | 40.784 | −52.663 | 1.00 | 114.71 | O |
| ATOM | 8044 | CB | GLN | D | 229 | 63.822 | 39.426 | −50.073 | 1.00 | 88.44 | C |
| ATOM | 8045 | CG | GLN | D | 229 | 65.078 | 39.137 | −49.299 | 1.00 | 85.28 | C |
| ATOM | 8046 | CD | GLN | D | 229 | 65.051 | 37.744 | −48.713 | 1.00 | 87.50 | C |
| ATOM | 8047 | OE1 | GLN | D | 229 | 65.547 | 37.509 | −47.608 | 1.00 | 83.56 | O |
| ATOM | 8048 | NE2 | GLN | D | 229 | 64.454 | 36.806 | −49.449 | 1.00 | 87.34 | N |
| ATOM | 8049 | N | ILE | D | 230 | 63.382 | 39.796 | −53.021 | 1.00 | 93.51 | N |
| ATOM | 8050 | CA | ILE | D | 230 | 63.658 | 39.324 | −54.375 | 1.00 | 95.37 | C |
| ATOM | 8051 | C | ILE | D | 230 | 62.692 | 38.190 | −54.638 | 1.00 | 80.95 | C |
| ATOM | 8052 | O | ILE | D | 230 | 61.497 | 38.408 | −54.812 | 1.00 | 91.66 | O |
| ATOM | 8053 | CB | ILE | D | 230 | 65.087 | 38.771 | −54.517 | 1.00 | 104.11 | C |
| ATOM | 8054 | CG1 | ILE | D | 230 | 65.307 | 38.190 | −55.918 | 1.00 | 99.04 | C |
| ATOM | 8055 | CG2 | ILE | D | 230 | 65.351 | 37.706 | −53.455 | 1.00 | 90.14 | C |
| ATOM | 8056 | CD1 | ILE | D | 230 | 64.831 | 36.756 | −56.073 | 1.00 | 83.25 | C |
| ATOM | 8057 | N | ARG | D | 267 | 66.556 | 29.458 | −54.852 | 1.00 | 95.95 | N |
| ATOM | 8058 | CA | ARG | D | 267 | 65.724 | 28.261 | −54.835 | 1.00 | 102.74 | C |
| ATOM | 8059 | C | ARG | D | 267 | 65.047 | 28.193 | −53.475 | 1.00 | 100.85 | C |
| ATOM | 8060 | O | ARG | D | 267 | 64.219 | 27.317 | −53.210 | 1.00 | 91.34 | O |
| ATOM | 8061 | CB | ARG | D | 267 | 66.571 | 27.008 | −55.052 | 1.00 | 111.25 | C |
| ATOM | 8062 | CG | ARG | D | 267 | 67.133 | 26.864 | −56.459 | 1.00 | 129.89 | C |
| ATOM | 8063 | CD | ARG | D | 267 | 66.062 | 26.435 | −57.460 | 1.00 | 130.40 | C |
| ATOM | 8064 | NE | ARG | D | 267 | 66.618 | 26.229 | −58.796 | 1.00 | 138.47 | N |
| ATOM | 8065 | CZ | ARG | D | 267 | 65.936 | 25.752 | −59.834 | 1.00 | 131.25 | C |
| ATOM | 8066 | NH1 | ARG | D | 267 | 64.658 | 25.422 | −59.702 | 1.00 | 116.16 | N |
| ATOM | 8067 | NH2 | ARG | D | 267 | 66.536 | 25.601 | −61.007 | 1.00 | 133.63 | N |
| ATOM | 8068 | N | GLU | D | 268 | 65.424 | 29.133 | −52.615 | 1.00 | 99.56 | N |
| ATOM | 8069 | CA | GLU | D | 268 | 64.847 | 29.262 | −51.287 | 1.00 | 78.15 | C |
| ATOM | 8070 | C | GLU | D | 268 | 63.386 | 29.690 | −51.374 | 1.00 | 73.17 | C |
| ATOM | 8071 | O | GLU | D | 268 | 62.564 | 29.268 | −50.565 | 1.00 | 71.46 | O |
| ATOM | 8072 | CB | GLU | D | 268 | 65.641 | 30.282 | −50.472 | 1.00 | 91.39 | C |
| ATOM | 8073 | CG | GLU | D | 268 | 67.142 | 30.029 | −50.462 | 1.00 | 119.84 | C |
| ATOM | 8074 | CD | GLU | D | 268 | 67.529 | 28.835 | −49.609 | 1.00 | 131.25 | C |
| ATOM | 8075 | OE1 | GLU | D | 268 | 66.707 | 28.412 | −48.768 | 1.00 | 132.47 | O |
| ATOM | 8076 | OE2 | GLU | D | 268 | 68.657 | 28.323 | −49.777 | 1.00 | 131.55 | O |
| ATOM | 8077 | N | HIS | D | 269 | 63.068 | 30.526 | −52.359 | 1.00 | 74.28 | N |
| ATOM | 8078 | CA | HIS | D | 269 | 61.691 | 30.953 | −52.578 | 1.00 | 72.83 | C |
| ATOM | 8079 | C | HIS | D | 269 | 60.830 | 29.795 | −53.055 | 1.00 | 57.35 | C |
| ATOM | 8080 | O | HIS | D | 269 | 59.610 | 29.818 | −52.919 | 1.00 | 67.94 | O |
| ATOM | 8081 | CB | HIS | D | 269 | 61.632 | 32.123 | −53.565 | 1.00 | 63.99 | C |
| ATOM | 8082 | CG | HIS | D | 269 | 62.125 | 33.407 | −52.981 | 1.00 | 88.92 | C |
| ATOM | 8083 | ND1 | HIS | D | 269 | 61.715 | 34.650 | −53.448 | 1.00 | 92.00 | N |
| ATOM | 8084 | CD2 | HIS | D | 269 | 62.970 | 33.661 | −51.960 | 1.00 | 94.74 | C |
| ATOM | 8085 | CE1 | HIS | D | 269 | 62.305 | 35.592 | −52.749 | 1.00 | 90.46 | C |
| ATOM | 8086 | NE2 | HIS | D | 269 | 63.075 | 35.024 | −51.829 | 1.00 | 92.43 | N |
| ATOM | 8087 | N | LYS | D | 270 | 61.470 | 28.770 | −53.595 | 1.00 | 55.39 | N |
| ATOM | 8088 | CA | LYS | D | 270 | 60.741 | 27.590 | −54.040 | 1.00 | 72.56 | C |
| ATOM | 8089 | C | LYS | D | 270 | 60.438 | 26.669 | −52.861 | 1.00 | 72.24 | C |
| ATOM | 8090 | O | LYS | D | 270 | 59.406 | 25.995 | −52.834 | 1.00 | 65.48 | O |
| ATOM | 8091 | CB | LYS | D | 270 | 61.515 | 26.872 | −55.144 | 1.00 | 73.08 | C |
| ATOM | 8092 | CG | LYS | D | 270 | 61.745 | 27.776 | −56.354 | 1.00 | 102.52 | C |
| ATOM | 8093 | CD | LYS | D | 270 | 62.772 | 27.225 | −57.332 | 1.00 | 126.84 | C |
| ATOM | 8094 | CE | LYS | D | 270 | 63.046 | 28.219 | −58.461 | 1.00 | 125.90 | C |
| ATOM | 8095 | NZ | LYS | D | 270 | 61.813 | 28.571 | −59.228 | 1.00 | 127.49 | N |
| ATOM | 8096 | N | ALA | D | 271 | 61.333 | 26.664 | −51.879 | 1.00 | 63.57 | N |
| ATOM | 8097 | CA | ALA | D | 271 | 61.094 | 25.958 | −50.630 | 1.00 | 45.39 | C |
| ATOM | 8098 | C | ALA | D | 271 | 60.034 | 26.688 | −49.796 | 1.00 | 55.18 | C |
| ATOM | 8099 | O | ALA | D | 271 | 59.201 | 26.057 | −49.137 | 1.00 | 50.07 | O |
| ATOM | 8100 | CB | ALA | D | 271 | 62.381 | 25.825 | −49.856 | 1.00 | 50.03 | C |
| ATOM | 8101 | N | LEU | D | 272 | 60.069 | 28.018 | −49.832 | 1.00 | 43.08 | N |
| ATOM | 8102 | CA | LEU | D | 272 | 59.062 | 28.833 | −49.166 | 1.00 | 44.91 | C |
| ATOM | 8103 | C | LEU | D | 272 | 57.695 | 28.725 | −49.831 | 1.00 | 47.47 | C |
| ATOM | 8104 | O | LEU | D | 272 | 56.666 | 28.861 | −49.171 | 1.00 | 44.87 | O |
| ATOM | 8105 | CB | LEU | D | 272 | 59.487 | 30.299 | −49.134 | 1.00 | 49.57 | C |
| ATOM | 8106 | CG | LEU | D | 272 | 60.616 | 30.647 | −48.168 | 1.00 | 47.03 | C |
| ATOM | 8107 | CD1 | LEU | D | 272 | 60.810 | 32.143 | −48.148 | 1.00 | 45.70 | C |
| ATOM | 8108 | CD2 | LEU | D | 272 | 60.312 | 30.123 | −46.777 | 1.00 | 29.86 | C |
| ATOM | 8109 | N | LYS | D | 273 | 57.672 | 28.490 | −51.138 | 1.00 | 57.41 | N |
| ATOM | 8110 | CA | LYS | D | 273 | 56.397 | 28.428 | −51.839 | 1.00 | 52.99 | C |
| ATOM | 8111 | C | LYS | D | 273 | 55.667 | 27.141 | −51.482 | 1.00 | 50.91 | C |
| ATOM | 8112 | O | LYS | D | 273 | 54.438 | 27.095 | −51.452 | 1.00 | 43.18 | O |
| ATOM | 8113 | CB | LYS | D | 273 | 56.581 | 28.547 | −53.349 | 1.00 | 51.54 | C |
| ATOM | 8114 | CG | LYS | D | 273 | 55.431 | 29.299 | −54.014 | 1.00 | 83.76 | C |
| ATOM | 8115 | CD | LYS | D | 273 | 54.958 | 28.638 | −55.306 | 1.00 | 81.99 | C |
| ATOM | 8116 | CE | LYS | D | 273 | 53.869 | 29.471 | −55.968 | 1.00 | 73.40 | C |
| ATOM | 8117 | NZ | LYS | D | 273 | 53.316 | 28.808 | −57.177 | 1.00 | 88.35 | N |
| ATOM | 8118 | N | THR | D | 274 | 56.439 | 26.099 | −51.197 | 1.00 | 51.68 | N |
| ATOM | 8119 | CA | THR | D | 274 | 55.881 | 24.824 | −50.775 | 1.00 | 44.79 | C |
| ATOM | 8120 | C | THR | D | 274 | 55.340 | 24.914 | −49.348 | 1.00 | 44.75 | C |
| ATOM | 8121 | O | THR | D | 274 | 54.276 | 24.377 | −49.046 | 1.00 | 41.55 | O |
| ATOM | 8122 | CB | THR | D | 274 | 56.935 | 23.718 | −50.858 | 1.00 | 39.54 | C |
| ATOM | 8123 | OG1 | THR | D | 274 | 57.486 | 23.696 | −52.179 | 1.00 | 56.89 | O |
| ATOM | 8124 | CG2 | THR | D | 274 | 56.321 | 22.365 | −50.553 | 1.00 | 47.87 | C |
| ATOM | 8125 | N | LEU | D | 275 | 56.073 | 25.599 | −48.475 | 1.00 | 41.41 | N |
| ATOM | 8126 | CA | LEU | D | 275 | 55.604 | 25.819 | −47.112 | 1.00 | 35.53 | C |
| ATOM | 8127 | C | LEU | D | 275 | 54.289 | 26.565 | −47.157 | 1.00 | 38.08 | C |
| ATOM | 8128 | O | LEU | D | 275 | 53.365 | 26.233 | −46.416 | 1.00 | 34.76 | O |
| ATOM | 8129 | CB | LEU | D | 275 | 56.629 | 26.596 | −46.287 | 1.00 | 30.71 | C |
| ATOM | 8130 | CG | LEU | D | 275 | 57.925 | 25.825 | −46.006 | 1.00 | 45.29 | C |
| ATOM | 8131 | CD1 | LEU | D | 275 | 58.855 | 26.595 | −45.055 | 1.00 | 36.74 | C |
| ATOM | 8132 | CD2 | LEU | D | 275 | 57.619 | 24.419 | −45.462 | 1.00 | 32.17 | C |
| ATOM | 8133 | N | GLY | D | 276 | 54.210 | 27.561 | −48.040 | 1.00 | 33.71 | N |
| ATOM | 8134 | CA | GLY | D | 276 | 52.981 | 28.308 | −48.256 | 1.00 | 34.24 | C |
| ATOM | 8135 | C | GLY | D | 276 | 51.842 | 27.441 | −48.771 | 1.00 | 37.00 | C |
| ATOM | 8136 | O | GLY | D | 276 | 50.707 | 27.587 | −48.338 | 1.00 | 32.99 | O |
| ATOM | 8137 | N | ILE | D | 277 | 52.143 | 26.534 | −49.696 | 1.00 | 38.32 | N |
| ATOM | 8138 | CA | ILE | D | 277 | 51.137 | 25.616 | −50.219 | 1.00 | 32.04 | C |
| ATOM | 8139 | C | ILE | D | 277 | 50.618 | 24.686 | −49.124 | 1.00 | 32.37 | C |
| ATOM | 8140 | O | ILE | D | 277 | 49.417 | 24.459 | −49.020 | 1.00 | 34.46 | O |
| ATOM | 8141 | CB | ILE | D | 277 | 51.676 | 24.800 | −51.423 | 1.00 | 36.08 | C |
| ATOM | 8142 | CG1 | ILE | D | 277 | 51.826 | 25.704 | −52.648 | 1.00 | 32.33 | C |
| ATOM | 8143 | CG2 | ILE | D | 277 | 50.752 | 23.636 | −51.768 | 1.00 | 22.56 | C |
| ATOM | 8144 | CD1 | ILE | D | 277 | 52.909 | 25.266 | −53.614 | 1.00 | 42.34 | C |
| ATOM | 8145 | N | ILE | D | 278 | 51.522 | 24.162 | −48.304 | 1.00 | 33.99 | N |
| ATOM | 8146 | CA | ILE | D | 278 | 51.153 | 23.299 | −47.184 | 1.00 | 32.70 | C |
| ATOM | 8147 | C | ILE | D | 278 | 50.172 | 23.985 | −46.216 | 1.00 | 38.70 | C |
| ATOM | 8148 | O | ILE | D | 278 | 49.197 | 23.381 | −45.765 | 1.00 | 31.55 | O |
| ATOM | 8149 | CB | ILE | D | 278 | 52.405 | 22.848 | −46.418 | 1.00 | 36.33 | C |
| ATOM | 8150 | CG1 | ILE | D | 278 | 53.239 | 21.917 | −47.287 | 1.00 | 52.35 | C |
| ATOM | 8151 | CG2 | ILE | D | 278 | 52.037 | 22.093 | −45.174 | 1.00 | 36.57 | C |
| ATOM | 8152 | CD1 | ILE | D | 278 | 52.659 | 20.543 | −47.390 | 1.00 | 43.55 | C |
| ATOM | 8153 | N | MET | D | 279 | 50.432 | 25.254 | −45.916 | 1.00 | 38.93 | N |
| ATOM | 8154 | CA | MET | D | 279 | 49.624 | 26.007 | −44.969 | 1.00 | 31.66 | C |
| ATOM | 8155 | C | MET | D | 279 | 48.284 | 26.407 | −45.558 | 1.00 | 33.52 | C |
| ATOM | 8156 | O | MET | D | 279 | 47.277 | 26.446 | −44.852 | 1.00 | 38.36 | O |
| ATOM | 8157 | CB | MET | D | 279 | 50.370 | 27.254 | −44.500 | 1.00 | 28.69 | C |
| ATOM | 8158 | CG | MET | D | 279 | 51.667 | 26.967 | −43.764 | 1.00 | 32.04 | C |
| ATOM | 8159 | SD | MET | D | 279 | 52.567 | 28.472 | −43.315 | 1.00 | 41.24 | S |
| ATOM | 8160 | CE | MET | D | 279 | 54.030 | 27.745 | −42.590 | 1.00 | 34.41 | C |
| ATOM | 8161 | N | GLY | D | 280 | 48.275 | 26.712 | −46.849 | 1.00 | 29.19 | N |
| ATOM | 8162 | CA | GLY | D | 280 | 47.048 | 27.074 | −47.533 | 1.00 | 28.74 | C |
| ATOM | 8163 | C | GLY | D | 280 | 46.112 | 25.894 | −47.692 | 1.00 | 29.40 | C |
| ATOM | 8164 | O | GLY | D | 280 | 44.909 | 26.010 | −47.463 | 1.00 | 30.31 | O |
| ATOM | 8165 | N | VAL | D | 281 | 46.667 | 24.753 | −48.085 | 1.00 | 25.84 | N |
| ATOM | 8166 | CA | VAL | D | 281 | 45.872 | 23.545 | −48.269 | 1.00 | 32.79 | C |
| ATOM | 8167 | C | VAL | D | 281 | 45.304 | 23.051 | −46.932 | 1.00 | 36.35 | C |
| ATOM | 8168 | O | VAL | D | 281 | 44.179 | 22.543 | −46.873 | 1.00 | 29.45 | O |
| ATOM | 8169 | CB | VAL | D | 281 | 46.682 | 22.414 | −48.959 | 1.00 | 31.97 | C |
| ATOM | 8170 | CG1 | VAL | D | 281 | 45.845 | 21.174 | −49.109 | 1.00 | 25.43 | C |
| ATOM | 8171 | CG2 | VAL | D | 281 | 47.153 | 22.856 | −50.322 | 1.00 | 28.74 | C |
| ATOM | 8172 | N | PHE | D | 282 | 46.077 | 23.209 | −45.859 | 1.00 | 28.77 | N |
| ATOM | 8173 | CA | PHE | D | 282 | 45.590 | 22.839 | −44.536 | 1.00 | 27.17 | C |
| ATOM | 8174 | C | PHE | D | 282 | 44.363 | 23.674 | −44.148 | 1.00 | 29.02 | C |
| ATOM | 8175 | O | PHE | D | 282 | 43.364 | 23.148 | −43.660 | 1.00 | 26.95 | O |
| ATOM | 8176 | CB | PHE | D | 282 | 46.689 | 22.988 | −43.490 | 1.00 | 23.81 | C |
| ATOM | 8177 | CG | PHE | D | 282 | 46.222 | 22.738 | −42.074 | 1.00 | 28.53 | C |
| ATOM | 8178 | CD1 | PHE | D | 282 | 46.366 | 21.475 | −41.491 | 1.00 | 22.01 | C |
| ATOM | 8179 | CD2 | PHE | D | 282 | 45.647 | 23.767 | −41.324 | 1.00 | 16.61 | C |
| ATOM | 8180 | CE1 | PHE | D | 282 | 45.950 | 21.244 | −40.183 | 1.00 | 19.32 | C |
| ATOM | 8181 | CE2 | PHE | D | 282 | 45.220 | 23.542 | −40.029 | 1.00 | 20.06 | C |
| ATOM | 8182 | CZ | PHE | D | 282 | 45.376 | 22.282 | −39.451 | 1.00 | 21.15 | C |
| ATOM | 8183 | N | THR | D | 283 | 44.452 | 24.978 | −44.370 | 1.00 | 25.35 | N |
| ATOM | 8184 | CA | THR | D | 283 | 43.338 | 25.876 | −44.137 | 1.00 | 24.04 | C |
| ATOM | 8185 | C | THR | D | 283 | 42.128 | 25.488 | −44.989 | 1.00 | 30.29 | C |
| ATOM | 8186 | O | THR | D | 283 | 41.033 | 25.305 | −44.472 | 1.00 | 37.88 | O |
| ATOM | 8187 | CB | THR | D | 283 | 43.738 | 27.339 | −44.429 | 1.00 | 26.75 | C |
| ATOM | 8188 | OG1 | THR | D | 283 | 44.926 | 27.657 | −43.698 | 1.00 | 26.79 | O |
| ATOM | 8189 | CG2 | THR | D | 283 | 42.620 | 28.309 | −44.036 | 1.00 | 26.16 | C |
| ATOM | 8190 | N | LEU | D | 284 | 42.316 | 25.358 | −46.293 | 1.00 | 28.75 | N |
| ATOM | 8191 | CA | LEU | D | 284 | 41.218 | 24.950 | −47.153 | 1.00 | 36.42 | C |
| ATOM | 8192 | C | LEU | D | 284 | 40.555 | 23.637 | −46.712 | 1.00 | 33.53 | C |
| ATOM | 8193 | O | LEU | D | 284 | 39.388 | 23.415 | −46.994 | 1.00 | 37.51 | O |
| ATOM | 8194 | CB | LEU | D | 284 | 41.684 | 24.836 | −48.606 | 1.00 | 43.39 | C |
| ATOM | 8195 | CG | LEU | D | 284 | 42.164 | 26.138 | −49.247 | 1.00 | 51.02 | C |
| ATOM | 8196 | CD1 | LEU | D | 284 | 42.506 | 25.897 | −50.706 | 1.00 | 44.82 | C |
| ATOM | 8197 | CD2 | LEU | D | 284 | 41.119 | 27.241 | −49.098 | 1.00 | 39.51 | C |
| ATOM | 8198 | N | CYS | D | 285 | 41.287 | 22.768 | −46.026 | 1.00 | 22.25 | N |
| ATOM | 8199 | CA | CYS | D | 285 | 40.761 | 21.445 | −45.722 | 1.00 | 22.27 | C |
| ATOM | 8200 | C | CYS | D | 285 | 40.018 | 21.350 | −44.400 | 1.00 | 30.31 | C |
| ATOM | 8201 | O | CYS | D | 285 | 39.223 | 20.430 | −44.206 | 1.00 | 34.61 | O |
| ATOM | 8202 | CB | CYS | D | 285 | 41.883 | 20.413 | −45.710 | 1.00 | 31.81 | C |
| ATOM | 8203 | SG | CYS | D | 285 | 42.320 | 19.712 | −47.299 | 1.00 | 38.11 | S |
| ATOM | 8204 | N | TRP | D | 286 | 40.289 | 22.279 | −43.485 | 1.00 | 30.28 | N |
| ATOM | 8205 | CA | TRP | D | 286 | 39.764 | 22.206 | −42.116 | 1.00 | 21.92 | C |
| ATOM | 8206 | C | TRP | D | 286 | 38.831 | 23.356 | −41.767 | 1.00 | 29.20 | C |
| ATOM | 8207 | O | TRP | D | 286 | 37.944 | 23.212 | −40.934 | 1.00 | 26.83 | O |
| ATOM | 8208 | CB | TRP | D | 286 | 40.900 | 22.166 | −41.108 | 1.00 | 18.07 | C |
| ATOM | 8209 | CG | TRP | D | 286 | 41.431 | 20.802 | −40.893 | 1.00 | 20.32 | C |
| ATOM | 8210 | CD1 | TRP | D | 286 | 42.673 | 20.352 | −41.213 | 1.00 | 17.32 | C |
| ATOM | 8211 | CD2 | TRP | D | 286 | 40.731 | 19.690 | −40.321 | 1.00 | 19.10 | C |
| ATOM | 8212 | NE1 | TRP | D | 286 | 42.796 | 19.029 | −40.872 | 1.00 | 18.62 | N |
| ATOM | 8213 | CE2 | TRP | D | 286 | 41.618 | 18.598 | −40.320 | 1.00 | 16.38 | C |
| ATOM | 8214 | CE3 | TRP | D | 286 | 39.446 | 19.513 | −39.812 | 1.00 | 16.48 | C |
| ATOM | 8215 | CZ2 | TRP | D | 266 | 41.263 | 17.347 | −39.829 | 1.00 | 14.33 | C |
| ATOM | 8216 | CZ3 | TRP | D | 286 | 39.098 | 18.273 | −39.318 | 1.00 | 20.59 | C |
| ATOM | 8217 | CH2 | TRP | D | 286 | 40.006 | 17.205 | −39.331 | 1.00 | 18.53 | C |
| ATOM | 8218 | N | LEU | D | 287 | 39.037 | 24.503 | −42.400 | 1.00 | 28.13 | N |
| ATOM | 8219 | CA | LEU | D | 287 | 38.178 | 25.650 | −42.156 | 1.00 | 28.72 | C |
| ATOM | 8220 | C | LEU | D | 287 | 36.688 | 25.323 | −42.367 | 1.00 | 30.33 | C |
| ATOM | 8221 | O | LEU | D | 287 | 35.846 | 25.779 | −41.601 | 1.00 | 34.00 | O |
| ATOM | 8222 | CB | LEU | D | 287 | 38.622 | 26.846 | −43.002 | 1.00 | 31.16 | C |
| ATOM | 8223 | CG | LEU | D | 287 | 37.939 | 28.187 | −42.733 | 1.00 | 37.64 | C |
| ATOM | 8224 | CD1 | LEU | D | 287 | 37.811 | 28.444 | −41.242 | 1.00 | 28.41 | C |
| ATOM | 8225 | CD2 | LEU | D | 287 | 38.706 | 29.310 | −43.402 | 1.00 | 36.84 | C |
| ATOM | 8226 | N | PRO | D | 288 | 36.353 | 24.527 | −43.396 | 1.00 | 32.72 | N |
| ATOM | 8227 | CA | PRO | D | 288 | 34.920 | 24.212 | −43.534 | 1.00 | 30.90 | C |
| ATOM | 8228 | C | PRO | D | 288 | 34.376 | 23.507 | −42.294 | 1.00 | 32.43 | C |
| ATOM | 8229 | O | PRO | D | 288 | 33.370 | 23.935 | −41.736 | 1.00 | 36.00 | O |
| ATOM | 8230 | CB | PRO | D | 288 | 34.866 | 23.274 | −44.753 | 1.00 | 26.84 | C |
| ATOM | 8231 | CG | PRO | D | 288 | 36.119 | 23.585 | −45.534 | 1.00 | 29.49 | C |
| ATOM | 8232 | CD | PRO | D | 288 | 37.159 | 23.994 | −44.513 | 1.00 | 31.14 | C |
| ATOM | 8233 | N | PHE | D | 289 | 35.043 | 22.447 | −41.856 | 1.00 | 25.46 | N |
| ATOM | 8234 | CA | PHE | D | 289 | 34.569 | 21.695 | −40.705 | 1.00 | 24.09 | C |
| ATOM | 8235 | C | PHE | D | 289 | 34.424 | 22.533 | −39.418 | 1.00 | 33.01 | C |
| ATOM | 8236 | O | PHE | D | 289 | 33.520 | 22.275 | −38.623 | 1.00 | 35.49 | O |
| ATOM | 8237 | CB | PHE | D | 289 | 35.452 | 20.471 | −40.472 | 1.00 | 22.53 | C |
| ATOM | 8238 | CG | PHE | D | 289 | 35.278 | 19.841 | −39.116 | 1.00 | 32.15 | C |
| ATOM | 8239 | CD1 | PHE | D | 289 | 34.469 | 18.721 | −38.952 | 1.00 | 32.57 | C |
| ATOM | 8240 | CD2 | PHE | D | 289 | 35.932 | 20.364 | −37.994 | 1.00 | 28.76 | C |
| ATOM | 8241 | CE1 | PHE | D | 289 | 34.310 | 18.122 | −37.697 | 1.00 | 26.39 | C |
| ATOM | 8242 | CE2 | PHE | D | 289 | 35.780 | 19.774 | −36.737 | 1.00 | 27.71 | C |
| ATOM | 8243 | CZ | PHE | D | 289 | 34.972 | 18.648 | −36.592 | 1.00 | 27.96 | C |
| ATOM | 8244 | N | PHE | D | 290 | 35.301 | 23.518 | −39.207 | 1.00 | 32.46 | N |
| ATOM | 8245 | CA | PHE | D | 290 | 35.225 | 24.371 | −38.008 | 1.00 | 31.02 | C |
| ATOM | 8246 | C | PHE | D | 290 | 34.211 | 25.481 | −38.179 | 1.00 | 33.73 | C |
| ATOM | 8247 | O | PHE | D | 290 | 33.626 | 25.948 | −37.204 | 1.00 | 33.39 | O |
| ATOM | 8248 | CB | PHE | D | 290 | 36.590 | 24.954 | −37.593 | 1.00 | 29.07 | C |
| ATOM | 8249 | CG | PHE | D | 290 | 37.490 | 23.953 | −36.917 | 1.00 | 25.36 | C |
| ATOM | 8250 | CD1 | PHE | D | 290 | 38.493 | 23.309 | −37.626 | 1.00 | 24.31 | C |
| ATOM | 8251 | CD2 | PHE | D | 290 | 37.315 | 23.634 | −35.591 | 1.00 | 25.53 | C |
| ATOM | 8252 | CE1 | PHE | D | 290 | 39.305 | 22.377 | −37.022 | 1.00 | 18.26 | C |
| ATOM | 8253 | CE2 | PHE | D | 290 | 38.126 | 22.691 | −34.984 | 1.00 | 31.45 | C |
| ATOM | 8254 | CZ | PHE | D | 290 | 39.116 | 22.065 | −35.702 | 1.00 | 24.13 | C |
| ATOM | 8255 | N | LEU | D | 291 | 33.992 | 25.905 | −39.417 | 1.00 | 30.31 | N |
| ATOM | 8256 | CA | LEU | D | 291 | 32.937 | 26.878 | −39.657 | 1.00 | 36.64 | C |
| ATOM | 8257 | C | LEU | D | 291 | 31.581 | 26.239 | −39.416 | 1.00 | 36.32 | C |
| ATOM | 8258 | O | LEU | D | 291 | 30.689 | 26.851 | −38.836 | 1.00 | 48.26 | O |
| ATOM | 8259 | CB | LEU | D | 291 | 33.028 | 27.498 | −41.050 | 1.00 | 35.77 | C |
| ATOM | 8260 | CG | LEU | D | 291 | 34.061 | 28.622 | −41.087 | 1.00 | 37.74 | C |
| ATOM | 8261 | CD1 | LEU | D | 291 | 33.956 | 29.433 | −42.365 | 1.00 | 24.96 | C |
| ATOM | 8262 | CD2 | LEU | D | 291 | 33.859 | 29.511 | −39.877 | 1.00 | 38.94 | C |
| ATOM | 8263 | N | VAL | D | 292 | 31.430 | 24.996 | −39.841 | 1.00 | 34.79 | N |
| ATOM | 8264 | CA | VAL | D | 292 | 30.170 | 24.286 | −39.640 | 1.00 | 37.13 | C |
| ATOM | 8265 | C | VAL | D | 292 | 29.985 | 23.935 | −38.164 | 1.00 | 33.65 | C |
| ATOM | 8266 | O | VAL | D | 292 | 28.875 | 23.663 | −37.694 | 1.00 | 35.33 | O |
| ATOM | 8267 | CB | VAL | D | 292 | 30.086 | 23.046 | −40.563 | 1.00 | 32.12 | C |
| ATOM | 8268 | CG1 | VAL | D | 292 | 29.679 | 21.797 | −39.798 | 1.00 | 30.86 | C |
| ATOM | 8269 | CG2 | VAL | D | 292 | 29.154 | 23.336 | −41.713 | 1.00 | 29.68 | C |
| ATOM | 8270 | N | ASN | D | 293 | 31.089 | 23.978 | −37.430 | 1.00 | 35.67 | N |
| ATOM | 8271 | CA | ASN | D | 293 | 31.070 | 23.658 | −36.010 | 1.00 | 37.64 | C |
| ATOM | 8272 | C | ASN | D | 293 | 30.554 | 24.823 | −35.174 | 1.00 | 34.94 | C |
| ATOM | 8273 | O | ASN | D | 293 | 29.899 | 24.612 | −34.172 | 1.00 | 29.15 | O |
| ATOM | 8274 | CB | ASN | D | 293 | 32.459 | 23.249 | −35.541 | 1.00 | 37.24 | C |
| ATOM | 8275 | CG | ASN | D | 293 | 32.426 | 22.076 | −34.590 | 1.00 | 46.27 | C |
| ATOM | 8276 | OD1 | ASN | D | 293 | 31.948 | 20.994 | −34.936 | 1.00 | 36.08 | O |
| ATOM | 8277 | ND2 | ASN | D | 293 | 32.952 | 22.277 | −33.386 | 1.00 | 54.55 | N |
| ATOM | 8278 | N | ILE | D | 294 | 30.854 | 26.048 | −35.598 | 1.00 | 39.38 | N |
| ATOM | 8279 | CA | ILE | D | 294 | 30.354 | 27.233 | −34.923 | 1.00 | 37.72 | C |
| ATOM | 8280 | C | ILE | D | 294 | 28.883 | 27.419 | −35.243 | 1.00 | 38.41 | C |
| ATOM | 8281 | O | ILE | D | 294 | 28.120 | 27.961 | −34.442 | 1.00 | 41.64 | O |
| ATOM | 8282 | CB | ILE | D | 294 | 31.140 | 28.493 | −35.337 | 1.00 | 40.31 | C |
| ATOM | 8283 | CG1 | ILE | D | 294 | 32.580 | 28.381 | −34.851 | 1.00 | 44.71 | C |
| ATOM | 8284 | CG2 | ILE | D | 294 | 30.503 | 29.769 | −34.770 | 1.00 | 29.81 | C |
| ATOM | 8285 | CD1 | ILE | D | 294 | 33.444 | 29.546 | −35.270 | 1.00 | 57.58 | C |
| ATOM | 8286 | N | VAL | D | 295 | 28.482 | 26.952 | −36.415 | 1.00 | 30.14 | N |
| ATOM | 8287 | CA | VAL | D | 295 | 27.091 | 27.066 | −36.826 | 1.00 | 32.81 | C |
| ATOM | 8288 | C | VAL | D | 295 | 26.175 | 26.121 | −36.032 | 1.00 | 36.89 | C |
| ATOM | 8289 | O | VAL | D | 295 | 25.057 | 26.499 | −35.691 | 1.00 | 30.26 | O |
| ATOM | 8290 | CB | VAL | D | 295 | 26.929 | 26.880 | −38.352 | 1.00 | 37.49 | C |
| ATOM | 8291 | CG1 | VAL | D | 295 | 25.460 | 26.798 | −38.748 | 1.00 | 39.69 | C |
| ATOM | 8292 | CG2 | VAL | D | 295 | 27.612 | 28.012 | −39.078 | 1.00 | 30.22 | C |
| ATOM | 8293 | N | ASN | D | 296 | 26.633 | 24.907 | −35.721 | 1.00 | 35.54 | N |
| ATOM | 8294 | CA | ASN | D | 296 | 25.816 | 24.032 | −34.867 | 1.00 | 40.82 | C |
| ATOM | 8295 | C | ASN | D | 296 | 25.673 | 24.567 | −33.462 | 1.00 | 28.01 | C |
| ATOM | 8296 | O | ASN | D | 296 | 24.809 | 24.141 | −32.715 | 1.00 | 27.18 | O |
| ATOM | 8297 | CB | ASN | D | 296 | 26.319 | 22.584 | −34.826 | 1.00 | 34.42 | C |
| ATOM | 8298 | CG | ASN | D | 296 | 25.699 | 21.736 | −35.921 | 1.00 | 71.75 | C |
| ATOM | 8299 | OD1 | ASN | D | 296 | 24.503 | 21.424 | −35.891 | 1.00 | 77.03 | O |
| ATOM | 8300 | ND2 | ASN | D | 296 | 26.501 | 21.386 | −36.914 | 1.00 | 76.98 | N |
| ATOM | 8301 | N | VAL | D | 297 | 26.535 | 25.505 | −33.110 | 1.00 | 27.97 | N |
| ATOM | 8302 | CA | VAL | D | 297 | 26.456 | 26.127 | −31.814 | 1.00 | 41.62 | C |
| ATOM | 8303 | C | VAL | D | 297 | 25.262 | 27.088 | −31.761 | 1.00 | 42.82 | C |
| ATOM | 8304 | O | VAL | D | 297 | 24.482 | 27.061 | −30.813 | 1.00 | 44.08 | O |
| ATOM | 8305 | CB | VAL | D | 297 | 27.778 | 26.831 | −31.440 | 1.00 | 43.32 | C |
| ATOM | 8306 | CG1 | VAL | D | 297 | 27.542 | 27.898 | −30.365 | 1.00 | 34.25 | C |
| ATOM | 8307 | CG2 | VAL | D | 297 | 28.804 | 25.801 | −30.967 | 1.00 | 28.85 | C |
| ATOM | 8308 | N | PHE | D | 298 | 25.101 | 27.926 | −32.777 | 1.00 | 37.96 | N |
| ATOM | 8309 | CA | PHE | D | 298 | 24.000 | 28.880 | −32.764 | 1.00 | 42.34 | C |
| ATOM | 8310 | C | PHE | D | 298 | 22.662 | 28.158 | −32.918 | 1.00 | 53.12 | C |
| ATOM | 8311 | O | PHE | D | 298 | 21.693 | 28.441 | −32.204 | 1.00 | 55.04 | O |
| ATOM | 8312 | CB | PHE | D | 298 | 24.216 | 29.982 | −33.810 | 1.00 | 44.58 | C |
| ATOM | 8313 | CG | PHE | D | 298 | 25.335 | 30.956 | −33.442 | 1.00 | 83.16 | C |
| ATOM | 8314 | CD1 | PHE | D | 298 | 26.508 | 30.498 | −32.832 | 1.00 | 71.03 | C |
| ATOM | 8315 | CD2 | PHE | D | 298 | 25.217 | 32.320 | −33.704 | 1.00 | 83.75 | C |
| ATOM | 8316 | CE1 | PHE | D | 298 | 27.540 | 31.373 | −32.487 | 1.00 | 51.53 | C |
| ATOM | 8317 | CE2 | PHE | D | 298 | 26.250 | 33.206 | −33.368 | 1.00 | 74.78 | C |
| ATOM | 8318 | CZ | PHE | D | 298 | 27.412 | 32.728 | −32.760 | 1.00 | 68.60 | C |
| ATOM | 8319 | N | ASN | D | 299 | 22.633 | 27.185 | −33.816 | 1.00 | 44.56 | N |
| ATOM | 8320 | CA | ASN | D | 299 | 21.435 | 26.412 | −34.057 | 1.00 | 33.98 | C |
| ATOM | 8321 | C | ASN | D | 299 | 21.871 | 25.024 | −34.481 | 1.00 | 36.44 | C |
| ATOM | 8322 | O | ASN | D | 299 | 22.383 | 24.831 | −35.585 | 1.00 | 38.36 | O |
| ATOM | 8323 | CB | ASN | D | 299 | 20.606 | 27.086 | −35.166 | 1.00 | 54.01 | C |
| ATOM | 8324 | CG | ASN | D | 299 | 19.194 | 26.493 | −35.326 | 1.00 | 62.23 | C |
| ATOM | 8325 | OD1 | ASN | D | 299 | 18.703 | 25.732 | −34.485 | 1.00 | 43.72 | O |
| ATOM | 8326 | ND2 | ASN | D | 299 | 18.536 | 26.861 | −36.421 | 1.00 | 67.04 | N |
| ATOM | 8327 | N | ARG | D | 300 | 21.714 | 24.055 | −33.592 | 1.00 | 42.13 | N |
| ATOM | 8328 | CA | ARG | D | 300 | 21.758 | 22.670 | −34.026 | 1.00 | 43.78 | C |
| ATOM | 8329 | C | ARG | D | 300 | 20.599 | 22.598 | −35.011 | 1.00 | 46.09 | C |
| ATOM | 8330 | O | ARG | D | 300 | 19.808 | 23.537 | −35.087 | 1.00 | 58.27 | O |
| ATOM | 8331 | CB | ARG | D | 300 | 21.576 | 21.737 | −32.831 | 1.00 | 49.64 | C |
| ATOM | 8332 | CG | ARG | D | 300 | 22.336 | 22.221 | −31.582 | 1.00 | 56.07 | C |
| ATOM | 8333 | CD | ARG | D | 300 | 22.864 | 21.078 | −30.707 | 1.00 | 78.85 | C |
| ATOM | 8334 | NE | ARG | D | 300 | 23.808 | 21.543 | −29.686 | 1.00 | 66.34 | N |
| ATOM | 8335 | CZ | ARG | D | 300 | 24.109 | 20.870 | −28.576 | 1.00 | 78.94 | C |
| ATOM | 8336 | NH1 | ARG | D | 300 | 23.539 | 19.696 | −28.325 | 1.00 | 72.71 | N |
| ATOM | 8337 | NH2 | ARG | D | 300 | 24.974 | 21.375 | −27.706 | 1.00 | 62.48 | N |
| ATOM | 8338 | N | ASP | D | 301 | 20.496 | 21.530 | −35.789 | 1.00 | 42.09 | N |
| ATOM | 8339 | CA | ASP | D | 301 | 19.451 | 21.455 | −36.836 | 1.00 | 52.98 | C |
| ATOM | 8340 | C | ASP | D | 301 | 19.744 | 22.302 | −38.096 | 1.00 | 45.95 | C |
| ATOM | 8341 | O | ASP | D | 301 | 19.362 | 21.921 | −39.201 | 1.00 | 57.49 | O |
| ATOM | 8342 | CB | ASP | D | 301 | 18.043 | 21.786 | −36.282 | 1.00 | 44.72 | C |
| ATOM | 8343 | CG | ASP | D | 301 | 17.587 | 20.809 | −35.179 | 1.00 | 60.78 | C |
| ATOM | 8344 | OD1 | ASP | D | 301 | 17.877 | 19.595 | −35.287 | 1.00 | 51.03 | O |
| ATOM | 8345 | OD2 | ASP | D | 301 | 16.934 | 21.253 | −34.200 | 1.00 | 55.47 | O |
| ATOM | 8346 | N | LEU | D | 302 | 20.431 | 23.429 | −37.936 | 1.00 | 46.47 | N |
| ATOM | 8347 | CA | LEU | D | 302 | 20.655 | 24.352 | −39.045 | 1.00 | 43.95 | C |
| ATOM | 8348 | C | LEU | D | 302 | 21.645 | 23.838 | −40.104 | 1.00 | 58.35 | C |
| ATOM | 8349 | O | LEU | D | 302 | 21.998 | 24.563 | −41.037 | 1.00 | 63.80 | O |
| ATOM | 8350 | CB | LEU | D | 302 | 21.117 | 25.712 | −38.513 | 1.00 | 49.78 | C |
| ATOM | 8351 | CG | LEU | D | 302 | 20.915 | 26.918 | −39.435 | 1.00 | 67.21 | C |
| ATOM | 8352 | CD1 | LEU | D | 302 | 19.505 | 27.459 | −39.293 | 1.00 | 73.77 | C |
| ATOM | 8353 | CD2 | LEU | D | 302 | 21.927 | 28.011 | −39.144 | 1.00 | 67.12 | C |
| ATOM | 8354 | N | VAL | D | 303 | 22.092 | 22.594 | −39.963 | 1.00 | 54.17 | N |
| ATOM | 8355 | CA | VAL | D | 303 | 23.069 | 22.020 | −40.891 | 1.00 | 47.07 | C |
| ATOM | 8356 | C | VAL | D | 303 | 23.161 | 20.504 | −40.726 | 1.00 | 44.79 | C |
| ATOM | 8357 | O | VAL | D | 303 | 23.342 | 19.999 | −39.621 | 1.00 | 48.74 | O |
| ATOM | 8358 | CB | VAL | D | 303 | 24.463 | 22.697 | −40.756 | 1.00 | 73.98 | C |
| ATOM | 8359 | CG1 | VAL | D | 303 | 25.551 | 21.683 | −40.417 | 1.00 | 52.06 | C |
| ATOM | 8360 | CG2 | VAL | D | 303 | 24.805 | 23.474 | −42.028 | 1.00 | 81.60 | C |
| ATOM | 8361 | N | PRO | D | 304 | 23.027 | 19.783 | −41.845 | 1.00 | 46.05 | N |
| ATOM | 8362 | CA | PRO | D | 304 | 22.713 | 18.351 | −41.960 | 1.00 | 42.69 | C |
| ATOM | 8363 | C | PRO | D | 304 | 23.895 | 17.438 | −41.708 | 1.00 | 47.95 | C |
| ATOM | 8364 | O | PRO | D | 304 | 24.967 | 17.631 | −42.284 | 1.00 | 53.21 | O |
| ATOM | 8365 | CB | PRO | D | 304 | 22.290 | 18.219 | −43.424 | 1.00 | 46.38 | C |
| ATOM | 8366 | CG | PRO | D | 304 | 23.097 | 19.282 | −44.112 | 1.00 | 46.70 | C |
| ATOM | 8367 | CD | PRO | D | 304 | 23.051 | 20.442 | −43.164 | 1.00 | 48.45 | C |
| ATOM | 8368 | N | ASP | D | 305 | 23.677 | 16.419 | −40.890 | 1.00 | 45.10 | N |
| ATOM | 8369 | CA | ASP | D | 305 | 24.745 | 15.513 | −40.478 | 1.00 | 50.60 | C |
| ATOM | 8370 | C | ASP | D | 305 | 25.688 | 15.105 | −41.619 | 1.00 | 56.72 | C |
| ATOM | 8371 | O | ASP | D | 305 | 26.907 | 15.051 | −41.436 | 1.00 | 58.04 | O |
| ATOM | 8372 | CB | ASP | D | 305 | 24.154 | 14.284 | −39.773 | 1.00 | 48.38 | C |
| ATOM | 8373 | CG | ASP | D | 305 | 23.285 | 14.661 | −38.563 | 1.00 | 82.04 | C |
| ATOM | 8374 | OD1 | ASP | D | 305 | 23.789 | 15.350 | −37.645 | 1.00 | 86.49 | O |
| ATOM | 8375 | OD2 | ASP | D | 305 | 22.095 | 14.274 | −38.526 | 1.00 | 78.84 | O |
| ATOM | 8376 | N | TRP | D | 306 | 25.135 | 14.841 | −42.799 | 1.00 | 53.90 | N |
| ATOM | 8377 | CA | TRP | D | 306 | 25.953 | 14.398 | −43.927 | 1.00 | 49.07 | C |
| ATOM | 8378 | C | TRP | D | 306 | 26.995 | 15.448 | −44.318 | 1.00 | 46.14 | C |
| ATOM | 8379 | O | TRP | D | 306 | 28.090 | 15.096 | −44.762 | 1.00 | 36.11 | O |
| ATOM | 8380 | CB | TRP | D | 306 | 25.079 | 14.020 | −45.143 | 1.00 | 39.31 | C |
| ATOM | 8381 | CG | TRP | D | 306 | 24.470 | 15.208 | −45.813 | 1.00 | 40.74 | C |
| ATOM | 8382 | CD1 | TRP | D | 306 | 23.219 | 15.703 | −45.610 | 1.00 | 39.79 | C |
| ATOM | 8383 | CD2 | TRP | D | 306 | 25.099 | 16.077 | −46.771 | 1.00 | 38.88 | C |
| ATOM | 8384 | NE1 | TRP | D | 306 | 23.024 | 16.822 | −46.387 | 1.00 | 48.43 | N |
| ATOM | 8385 | CE2 | TRP | D | 306 | 24.164 | 17.074 | −47.107 | 1.00 | 39.88 | C |
| ATOM | 8386 | CE3 | TRP | D | 306 | 26.364 | 16.113 | −47.368 | 1.00 | 48.17 | C |
| ATOM | 8387 | CZ2 | TRP | D | 306 | 24.446 | 18.092 | −48.023 | 1.00 | 37.91 | C |
| ATOM | 8388 | CZ3 | TRP | D | 306 | 26.644 | 17.130 | −48.286 | 1.00 | 48.31 | C |
| ATOM | 8389 | CH2 | TRP | D | 306 | 25.687 | 18.100 | −48.601 | 1.00 | 31.95 | C |
| ATOM | 8390 | N | LEU | D | 307 | 26.648 | 16.729 | −44.172 | 1.00 | 38.49 | N |
| ATOM | 8391 | CA | LEU | D | 307 | 27.580 | 17.795 | −44.510 | 1.00 | 39.42 | C |
| ATOM | 8392 | C | LEU | D | 307 | 28.702 | 17.827 | −43.475 | 1.00 | 45.64 | C |
| ATOM | 8393 | O | LEU | D | 307 | 29.819 | 18.285 | −43.738 | 1.00 | 36.32 | O |
| ATOM | 8394 | CB | LEU | D | 307 | 26.879 | 19.148 | −44.582 | 1.00 | 33.34 | C |
| ATOM | 8395 | CG | LEU | D | 307 | 27.823 | 20.314 | −44.915 | 1.00 | 38.63 | C |
| ATOM | 8396 | CD1 | LEU | D | 307 | 28.673 | 20.026 | −46.150 | 1.00 | 35.55 | C |
| ATOM | 8397 | CD2 | LEU | D | 307 | 27.052 | 21.606 | −45.101 | 1.00 | 47.66 | C |
| ATOM | 8398 | N | PHE | D | 308 | 28.401 | 17.314 | −42.292 | 1.00 | 44.70 | N |
| ATOM | 8399 | CA | PHE | D | 308 | 29.391 | 17.255 | −41.240 | 1.00 | 41.41 | C |
| ATOM | 8400 | C | PHE | D | 308 | 30.383 | 16.163 | −41.564 | 1.00 | 39.75 | C |
| ATOM | 8401 | O | PHE | D | 308 | 31.590 | 16.348 | −41.445 | 1.00 | 42.97 | O |
| ATOM | 8402 | CB | PHE | D | 308 | 28.728 | 16.970 | −39.902 | 1.00 | 38.18 | C |
| ATOM | 8403 | CG | PHE | D | 308 | 29.013 | 18.006 | −38.881 | 1.00 | 43.65 | C |
| ATOM | 8404 | CD1 | PHE | D | 308 | 27.987 | 18.730 | −38.303 | 1.00 | 47.48 | C |
| ATOM | 8405 | CD2 | PHE | D | 308 | 30.318 | 18.295 | −38.525 | 1.00 | 55.24 | C |
| ATOM | 8406 | CE1 | PHE | D | 308 | 28.261 | 19.704 | −37.364 | 1.00 | 45.97 | C |
| ATOM | 8407 | CE2 | PHE | D | 308 | 30.597 | 19.273 | −37.585 | 1.00 | 55.35 | C |
| ATOM | 8408 | CZ | PHE | D | 308 | 29.566 | 19.980 | −37.005 | 1.00 | 46.28 | C |
| ATOM | 8409 | N | VAL | D | 309 | 29.867 | 15.018 | −41.978 | 1.00 | 35.58 | N |
| ATOM | 8410 | CA | VAL | D | 309 | 30.725 | 13.900 | −42.287 | 1.00 | 28.15 | C |
| ATOM | 8411 | C | VAL | D | 309 | 31.634 | 14.283 | −43.436 | 1.00 | 32.48 | C |
| ATOM | 8412 | O | VAL | D | 309 | 32.825 | 13.989 | −43.412 | 1.00 | 33.33 | O |
| ATOM | 8413 | CB | VAL | D | 309 | 29.917 | 12.638 | −42.644 | 1.00 | 35.83 | C |
| ATOM | 8414 | CG1 | VAL | D | 309 | 30.810 | 11.608 | −43.288 | 1.00 | 32.75 | C |
| ATOM | 8415 | CG2 | VAL | D | 309 | 29.272 | 12.053 | −41.402 | 1.00 | 33.77 | C |
| ATOM | 8416 | N | ALA | D | 310 | 31.082 | 14.963 | −44.435 | 1.00 | 31.15 | N |
| ATOM | 8417 | CA | ALA | D | 310 | 31.865 | 15.312 | −45.622 | 1.00 | 34.56 | C |
| ATOM | 8418 | C | ALA | D | 310 | 32.997 | 16.263 | −45.270 | 1.00 | 30.94 | C |
| ATOM | 8419 | O | ALA | D | 310 | 34.155 | 15.996 | −45.586 | 1.00 | 26.65 | O |
| ATOM | 8420 | CB | ALA | D | 310 | 30.986 | 15.911 | −46.711 | 1.00 | 32.20 | C |
| ATOM | 8421 | N | PHE | D | 311 | 32.657 | 17.368 | −44.618 | 1.00 | 29.12 | N |
| ATOM | 8422 | CA | PHE | D | 311 | 33.668 | 18.327 | −44.194 | 1.00 | 30.86 | C |
| ATOM | 8423 | C | PHE | D | 311 | 34.729 | 17.725 | −43.278 | 1.00 | 23.35 | C |
| ATOM | 8424 | O | PHE | D | 311 | 35.905 | 18.089 | −43.358 | 1.00 | 20.32 | O |
| ATOM | 8425 | CB | PHE | D | 311 | 33.028 | 19.552 | −43.551 | 1.00 | 28.21 | C |
| ATOM | 8426 | CG | PHE | D | 311 | 32.433 | 20.502 | −44.543 | 1.00 | 32.17 | C |
| ATOM | 8427 | CD1 | PHE | D | 311 | 31.477 | 21.426 | −44.159 | 1.00 | 37.11 | C |
| ATOM | 8428 | CD2 | PHE | D | 311 | 32.817 | 20.457 | −45.869 | 1.00 | 29.14 | C |
| ATOM | 8429 | CE1 | PHE | D | 311 | 30.935 | 22.300 | −45.077 | 1.00 | 39.00 | C |
| ATOM | 8430 | CE2 | PHE | D | 311 | 32.278 | 21.330 | −46.784 | 1.00 | 31.00 | C |
| ATOM | 8431 | CZ | PHE | D | 311 | 31.339 | 22.252 | −46.393 | 1.00 | 25.18 | C |
| ATOM | 8432 | N | ASN | D | 312 | 34.320 | 16.800 | −42.419 | 1.00 | 21.90 | N |
| ATOM | 8433 | CA | ASN | D | 312 | 35.272 | 16.112 | −41.552 | 1.00 | 29.98 | C |
| ATOM | 8434 | C | ASN | D | 312 | 36.253 | 15.220 | −42.324 | 1.00 | 26.01 | C |
| ATOM | 8435 | O | ASN | D | 312 | 37.406 | 15.082 | −41.938 | 1.00 | 24.26 | O |
| ATOM | 8436 | CB | ASN | D | 312 | 34.549 | 15.300 | −40.471 | 1.00 | 24.35 | C |
| ATOM | 8437 | CG | ASN | D | 312 | 35.385 | 15.125 | −39.212 | 1.00 | 26.41 | C |
| ATOM | 8438 | OD1 | ASN | D | 312 | 34.886 | 14.680 | −38.181 | 1.00 | 30.82 | O |
| ATOM | 8439 | ND2 | ASN | D | 312 | 36.661 | 15.488 | −39.288 | 1.00 | 33.26 | N |
| ATOM | 8440 | N | TRP | D | 313 | 35.790 | 14.621 | −43.415 | 1.00 | 25.36 | N |
| ATOM | 8441 | CA | TRP | D | 313 | 36.652 | 13.783 | −44.224 | 1.00 | 22.66 | C |
| ATOM | 8442 | C | TRP | D | 313 | 37.504 | 14.617 | −45.152 | 1.00 | 22.05 | C |
| ATOM | 8443 | O | TRP | D | 313 | 38.587 | 14.194 | −45.550 | 1.00 | 25.44 | O |
| ATOM | 8444 | CB | TRP | D | 313 | 35.847 | 12.731 | −44.992 | 1.00 | 30.28 | C |
| ATOM | 8445 | CG | TRP | D | 313 | 35.433 | 11.628 | −44.079 | 1.00 | 40.01 | C |
| ATOM | 8446 | CD1 | TRP | D | 313 | 34.330 | 11.608 | −43.275 | 1.00 | 35.32 | C |
| ATOM | 8447 | CD2 | TRP | D | 313 | 36.140 | 10.409 | −43.820 | 1.00 | 36.48 | C |
| ATOM | 8448 | NE1 | TRP | D | 313 | 34.293 | 10.444 | −42.547 | 1.00 | 39.15 | N |
| ATOM | 8449 | CE2 | TRP | D | 313 | 35.392 | 9.688 | −42.858 | 1.00 | 38.86 | C |
| ATOM | 8450 | CE3 | TRP | D | 313 | 37.320 | 9.849 | −44.317 | 1.00 | 31.79 | C |
| ATOM | 8451 | CZ2 | TRP | D | 313 | 35.786 | 8.434 | −42.377 | 1.00 | 33.81 | C |
| ATOM | 8452 | CZ3 | TRP | D | 313 | 37.713 | 8.603 | −43.838 | 1.00 | 44.97 | C |
| ATOM | 8453 | CH2 | TRP | D | 313 | 36.944 | 7.909 | −42.873 | 1.00 | 35.86 | C |
| ATOM | 8454 | N | LEU | D | 314 | 37.021 | 15.807 | −45.489 | 1.00 | 14.95 | N |
| ATOM | 8455 | CA | LEU | D | 314 | 37.822 | 16.728 | −46.257 | 1.00 | 17.22 | C |
| ATOM | 8456 | C | LEU | D | 314 | 39.109 | 17.017 | −45.484 | 1.00 | 31.64 | C |
| ATOM | 8457 | O | LEU | D | 314 | 40.212 | 16.950 | −46.036 | 1.00 | 30.51 | O |
| ATOM | 8458 | CB | LEU | D | 314 | 37.062 | 18.020 | −46.517 | 1.00 | 19.32 | C |
| ATOM | 8459 | CG | LEU | D | 314 | 37.975 | 19.085 | −47.128 | 1.00 | 27.03 | C |
| ATOM | 8460 | CD1 | LEU | D | 314 | 38.663 | 18.549 | −48.387 | 1.00 | 17.77 | C |
| ATOM | 8461 | CD2 | LEU | D | 314 | 37.219 | 20.372 | −47.406 | 1.00 | 23.34 | C |
| ATOM | 8462 | N | GLY | D | 315 | 38.958 | 17.329 | −44.198 | 1.00 | 24.81 | N |
| ATOM | 8463 | CA | GLY | D | 315 | 40.087 | 17.510 | −43.301 | 1.00 | 24.03 | C |
| ATOM | 8464 | C | GLY | D | 315 | 40.976 | 16.294 | −43.073 | 1.00 | 23.21 | C |
| ATOM | 8465 | O | GLY | D | 315 | 42.188 | 16.435 | −42.954 | 1.00 | 20.76 | O |
| ATOM | 8466 | N | TYR | D | 316 | 40.401 | 15.100 | −42.991 | 1.00 | 21.10 | N |
| ATOM | 8467 | CA | TYR | D | 316 | 41.246 | 13.919 | −42.908 | 1.00 | 21.00 | C |
| ATOM | 8468 | C | TYR | D | 316 | 42.065 | 13.792 | −44.182 | 1.00 | 27.01 | C |
| ATOM | 8469 | O | TYR | D | 316 | 43.242 | 13.446 | −44.136 | 1.00 | 29.10 | O |
| ATOM | 8470 | CB | TYR | D | 316 | 40.452 | 12.631 | −42.721 | 1.00 | 25.78 | C |
| ATOM | 8471 | CG | TYR | D | 316 | 39.775 | 12.435 | −41.386 | 1.00 | 26.95 | C |
| ATOM | 8472 | CD1 | TYR | D | 316 | 38.600 | 11.697 | −41.308 | 1.00 | 25.65 | C |
| ATOM | 8473 | CD2 | TYR | D | 316 | 40.299 | 12.980 | −40.205 | 1.00 | 26.58 | C |
| ATOM | 8474 | CE1 | TYR | D | 316 | 37.958 | 11.500 | −40.106 | 1.00 | 32.38 | C |
| ATOM | 8475 | CE2 | TYR | D | 316 | 39.656 | 12.783 | −38.972 | 1.00 | 26.12 | C |
| ATOM | 8476 | CZ | TYR | D | 316 | 38.476 | 12.042 | −38.947 | 1.00 | 32.51 | C |
| ATOM | 8477 | OH | TYR | D | 316 | 37.785 | 11.816 | −37.791 | 1.00 | 25.19 | O |
| ATOM | 8478 | N | ALA | D | 317 | 41.447 | 14.058 | −45.329 | 1.00 | 27.93 | N |
| ATOM | 8479 | CA | ALA | D | 317 | 42.149 | 13.849 | −46.602 | 1.00 | 33.59 | C |
| ATOM | 8480 | C | ALA | D | 317 | 43.411 | 14.707 | −46.661 | 1.00 | 34.47 | C |
| ATOM | 8481 | O | ALA | D | 317 | 44.369 | 14.372 | −47.369 | 1.00 | 38.19 | O |
| ATOM | 8482 | CB | ALA | D | 317 | 41.233 | 14.104 | −47.807 | 1.00 | 24.37 | C |
| ATOM | 8483 | N | ASN | D | 318 | 43.410 | 15.799 | −45.897 | 1.00 | 25.75 | N |
| ATOM | 8484 | CA | ASN | D | 318 | 44.586 | 16.647 | −45.764 | 1.00 | 28.48 | C |
| ATOM | 8485 | C | ASN | D | 318 | 45.856 | 15.889 | −45.381 | 1.00 | 30.29 | C |
| ATOM | 8486 | O | ASN | D | 318 | 46.942 | 16.268 | −45.806 | 1.00 | 39.20 | O |
| ATOM | 8487 | CB | ASN | D | 318 | 44.331 | 17.763 | −44.757 | 1.00 | 29.33 | C |
| ATOM | 8488 | CG | ASN | D | 318 | 45.504 | 18.716 | −44.622 | 1.00 | 26.60 | C |
| ATOM | 8489 | OD1 | ASN | D | 318 | 45.559 | 19.738 | −45.293 | 1.00 | 36.52 | O |
| ATOM | 8490 | ND2 | ASN | D | 318 | 46.446 | 18.386 | −43.750 | 1.00 | 28.73 | N |
| ATOM | 8491 | N | SER | D | 319 | 45.730 | 14.828 | −44.587 | 1.00 | 23.22 | N |
| ATOM | 8492 | CA | SER | D | 319 | 46.899 | 14.029 | −44.187 | 1.00 | 26.95 | C |
| ATOM | 8493 | C | SER | D | 319 | 47.579 | 13.314 | −45.360 | 1.00 | 34.07 | C |
| ATOM | 8494 | O | SER | D | 319 | 48.720 | 12.864 | −45.244 | 1.00 | 31.33 | O |
| ATOM | 8495 | CB | SER | D | 319 | 46.532 | 12.992 | −43.121 | 1.00 | 27.35 | C |
| ATOM | 8496 | OG | SER | D | 319 | 46.196 | 13.600 | −41.883 | 1.00 | 25.20 | O |
| ATOM | 8497 | N | ALA | D | 320 | 46.875 | 13.209 | −46.486 | 1.00 | 37.70 | N |
| ATOM | 8498 | CA | ALA | D | 320 | 47.407 | 12.543 | −47.674 | 1.00 | 32.84 | C |
| ATOM | 8499 | C | ALA | D | 320 | 48.026 | 13.525 | −48.684 | 1.00 | 35.46 | C |
| ATOM | 8500 | O | ALA | D | 320 | 48.820 | 13.128 | −49.526 | 1.00 | 36.55 | O |
| ATOM | 8501 | CB | ALA | D | 320 | 46.331 | 11.705 | −48.328 | 1.00 | 24.83 | C |
| ATOM | 8502 | N | MET | D | 321 | 47.681 | 14.803 | −48.580 | 1.00 | 29.25 | N |
| ATOM | 8503 | CA | MET | D | 321 | 48.205 | 15.818 | −49.492 | 1.00 | 36.01 | C |
| ATOM | 8504 | C | MET | D | 321 | 49.678 | 16.201 | −49.355 | 1.00 | 36.05 | C |
| ATOM | 8505 | O | MET | D | 321 | 50.347 | 16.432 | −50.355 | 1.00 | 42.66 | O |
| ATOM | 8506 | CB | MET | D | 321 | 47.368 | 17.087 | −49.388 | 1.00 | 41.36 | C |
| ATOM | 8507 | CG | MET | D | 321 | 46.013 | 16.951 | −50.018 | 1.00 | 44.06 | C |
| ATOM | 8508 | SD | MET | D | 321 | 44.881 | 18.173 | −49.371 | 1.00 | 55.32 | S |
| ATOM | 8509 | CE | MET | D | 321 | 43.370 | 17.678 | −50.208 | 1.00 | 44.30 | C |
| ATOM | 8510 | N | ASN | D | 322 | 50.181 | 16.306 | −48.132 | 1.00 | 37.31 | N |
| ATOM | 8511 | CA | ASN | D | 322 | 51.528 | 16.831 | −47.930 | 1.00 | 36.72 | C |
| ATOM | 8512 | C | ASN | D | 322 | 52.633 | 16.143 | −48.742 | 1.00 | 41.76 | C |
| ATOM | 8513 | O | ASN | D | 322 | 53.401 | 16.821 | −49.428 | 1.00 | 44.82 | O |
| ATOM | 8514 | CB | ASN | D | 322 | 51.888 | 16.897 | −46.444 | 1.00 | 47.41 | C |
| ATOM | 8515 | CG | ASN | D | 322 | 51.290 | 18.111 | −45.751 | 1.00 | 40.90 | C |
| ATOM | 8516 | OD1 | ASN | D | 322 | 50.105 | 18.392 | −45.883 | 1.00 | 48.11 | O |
| ATOM | 8517 | ND2 | ASN | D | 322 | 52.113 | 18.833 | −45.005 | 1.00 | 37.79 | N |
| ATOM | 8518 | N | PRO | D | 323 | 52.718 | 14.801 | −48.680 | 1.00 | 44.70 | N |
| ATOM | 8519 | CA | PRO | D | 323 | 53.776 | 14.102 | −49.432 | 1.00 | 43.47 | C |
| ATOM | 8520 | C | PRO | D | 323 | 53.715 | 14.417 | −50.932 | 1.00 | 41.57 | C |
| ATOM | 8521 | O | PRO | D | 323 | 54.733 | 14.704 | −51.556 | 1.00 | 40.98 | O |
| ATOM | 8522 | CB | PRO | D | 323 | 53.476 | 12.616 | −49.177 | 1.00 | 37.92 | C |
| ATOM | 8523 | CG | PRO | D | 323 | 52.639 | 12.589 | −47.940 | 1.00 | 44.64 | C |
| ATOM | 8524 | CD | PRO | D | 323 | 51.831 | 13.862 | −47.968 | 1.00 | 48.51 | C |
| ATOM | 8525 | N | ILE | D | 324 | 52.518 | 14.366 | −51.499 | 1.00 | 32.52 | N |
| ATOM | 8526 | CA | ILE | D | 324 | 52.324 | 14.815 | −52.863 | 1.00 | 39.17 | C |
| ATOM | 8527 | C | ILE | D | 324 | 52.959 | 16.196 | −53.073 | 1.00 | 36.15 | C |
| ATOM | 8528 | O | ILE | D | 324 | 53.822 | 16.357 | −53.926 | 1.00 | 42.86 | O |
| ATOM | 8529 | CB | ILE | D | 324 | 50.819 | 14.823 | −53.244 | 1.00 | 40.12 | C |
| ATOM | 8530 | CG1 | ILE | D | 324 | 50.333 | 13.390 | −53.484 | 1.00 | 28.43 | C |
| ATOM | 8531 | CG2 | ILE | D | 324 | 50.570 | 15.697 | −54.472 | 1.00 | 32.13 | C |
| ATOM | 8532 | CD1 | ILE | D | 324 | 48.822 | 13.239 | −53.498 | 1.00 | 40.00 | C |
| ATOM | 8533 | N | ILE | D | 325 | 52.544 | 17.185 | −52.291 | 1.00 | 32.27 | N |
| ATOM | 8534 | CA | ILE | D | 325 | 53.085 | 18.536 | −52.429 | 1.00 | 44.78 | C |
| ATOM | 8535 | C | ILE | D | 325 | 54.631 | 18.612 | −52.384 | 1.00 | 48.42 | C |
| ATOM | 8536 | O | ILE | D | 325 | 55.231 | 19.468 | −53.043 | 1.00 | 36.31 | O |
| ATOM | 8537 | CB | ILE | D | 325 | 52.468 | 19.478 | −51.372 | 1.00 | 37.76 | C |
| ATOM | 8538 | CG1 | ILE | D | 325 | 50.957 | 19.528 | −51.541 | 1.00 | 30.30 | C |
| ATOM | 8539 | CG2 | ILE | D | 325 | 53.064 | 20.885 | −51.456 | 1.00 | 40.79 | C |
| ATOM | 8540 | CD1 | ILE | D | 325 | 50.240 | 20.226 | −50.399 | 1.00 | 32.30 | C |
| ATOM | 8541 | N | TYR | D | 326 | 55.268 | 17.728 | −51.612 | 1.00 | 34.91 | N |
| ATOM | 8542 | CA | TYR | D | 326 | 56.720 | 17.746 | −51.473 | 1.00 | 39.36 | C |
| ATOM | 8543 | C | TYR | D | 326 | 57.424 | 17.249 | −52.724 | 1.00 | 53.06 | C |
| ATOM | 8544 | O | TYR | D | 326 | 58.646 | 17.356 | −52.851 | 1.00 | 49.24 | O |
| ATOM | 8545 | CB | TYR | D | 326 | 57.183 | 16.871 | −50.316 | 1.00 | 50.03 | C |
| ATOM | 8546 | CG | TYR | D | 326 | 56.708 | 17.304 | −48.960 | 1.00 | 48.46 | C |
| ATOM | 8547 | CD1 | TYR | D | 326 | 56.427 | 16.360 | −47.977 | 1.00 | 45.38 | C |
| ATOM | 8548 | CD2 | TYR | D | 326 | 56.538 | 18.646 | −48.656 | 1.00 | 48.02 | C |
| ATOM | 8549 | CE1 | TYR | D | 326 | 55.995 | 16.738 | −46.734 | 1.00 | 38.79 | C |
| ATOM | 8550 | CE2 | TYR | D | 326 | 56.097 | 19.037 | −47.406 | 1.00 | 44.34 | C |
| ATOM | 8551 | CZ | TYR | D | 326 | 55.826 | 18.075 | −46.452 | 1.00 | 41.61 | C |
| ATOM | 8552 | OH | TYR | D | 326 | 55.378 | 18.444 | −45.210 | 1.00 | 44.51 | O |
| ATOM | 8553 | N | CYS | D | 327 | 56.661 | 16.669 | −53.638 | 1.00 | 50.06 | N |
| ATOM | 8554 | CA | CYS | D | 327 | 57.239 | 16.199 | −54.883 | 1.00 | 42.80 | C |
| ATOM | 8555 | C | CYS | D | 327 | 57.586 | 17.384 | −55.775 | 1.00 | 51.68 | C |
| ATOM | 8556 | O | CYS | D | 327 | 58.288 | 17.233 | −56.769 | 1.00 | 62.06 | O |
| ATOM | 8557 | CB | CYS | D | 327 | 56.301 | 15.217 | −55.565 | 1.00 | 35.52 | C |
| ATOM | 8558 | SG | CYS | D | 327 | 56.198 | 13.645 | −54.671 | 1.00 | 58.05 | S |
| ATOM | 8559 | N | ARG | D | 328 | 57.108 | 18.566 | −55.387 | 1.00 | 47.54 | N |
| ATOM | 8560 | CA | ARG | D | 328 | 57.484 | 19.822 | −56.025 | 1.00 | 49.29 | C |
| ATOM | 8561 | C | ARG | D | 328 | 58.977 | 20.076 | −55.897 | 1.00 | 57.89 | C |
| ATOM | 8562 | O | ARG | D | 328 | 59.591 | 20.676 | −56.774 | 1.00 | 62.66 | O |
| ATOM | 8563 | CB | ARG | D | 328 | 56.745 | 20.985 | −55.375 | 1.00 | 49.28 | C |
| ATOM | 8564 | CG | ARG | D | 328 | 55.276 | 21.091 | −55.731 | 1.00 | 60.98 | C |
| ATOM | 8565 | CD | ARG | D | 328 | 54.715 | 22.252 | −54.962 | 1.00 | 52.06 | C |
| ATOM | 8566 | NE | ARG | D | 328 | 55.796 | 23.190 | −54.701 | 1.00 | 56.38 | N |
| ATOM | 8567 | CZ | ARG | D | 328 | 55.961 | 24.338 | −55.345 | 1.00 | 63.00 | C |
| ATOM | 8568 | NH1 | ARG | D | 328 | 55.090 | 24.705 | −56.275 | 1.00 | 67.31 | N |
| ATOM | 8569 | NH2 | ARG | D | 328 | 56.984 | 25.128 | −55.042 | 1.00 | 61.76 | N |
| ATOM | 8570 | N | SER | D | 329 | 59.550 | 19.645 | −54.779 | 1.00 | 65.88 | N |
| ATOM | 8571 | CA | SER | D | 329 | 60.987 | 19.740 | −54.578 | 1.00 | 64.23 | C |
| ATOM | 8572 | C | SER | D | 329 | 61.676 | 18.622 | −55.331 | 1.00 | 70.12 | C |
| ATOM | 8573 | O | SER | D | 329 | 61.132 | 17.526 | −55.456 | 1.00 | 65.98 | O |
| ATOM | 8574 | CB | SER | D | 329 | 61.341 | 19.633 | −53.098 | 1.00 | 72.32 | C |
| ATOM | 8575 | OG | SER | D | 329 | 62.660 | 19.135 | −52.940 | 1.00 | 66.64 | O |
| ATOM | 8576 | N | PRO | D | 330 | 62.889 | 18.893 | −55.826 | 1.00 | 86.13 | N |
| ATOM | 8577 | CA | PRO | D | 330 | 63.672 | 17.918 | −56.589 | 1.00 | 75.79 | C |
| ATOM | 8578 | C | PRO | D | 330 | 64.286 | 16.871 | −55.671 | 1.00 | 72.07 | C |
| ATOM | 8579 | O | PRO | D | 330 | 64.447 | 15.719 | −56.072 | 1.00 | 63.80 | O |
| ATOM | 8580 | CB | PRO | D | 330 | 64.775 | 18.773 | −57.231 | 1.00 | 77.38 | C |
| ATOM | 8581 | CG | PRO | D | 330 | 64.386 | 20.222 | −56.967 | 1.00 | 91.37 | C |
| ATOM | 8582 | CD | PRO | D | 330 | 63.584 | 20.185 | −55.712 | 1.00 | 90.21 | C |
| ATOM | 8583 | N | ASP | D | 331 | 64.609 | 17.275 | −54.446 | 1.00 | 78.20 | N |
| ATOM | 8584 | CA | ASP | D | 331 | 65.266 | 16.400 | −53.478 | 1.00 | 75.09 | C |
| ATOM | 8585 | C | ASP | D | 331 | 64.360 | 15.272 | −52.999 | 1.00 | 67.51 | C |
| ATOM | 8586 | O | ASP | D | 331 | 64.754 | 14.110 | −53.009 | 1.00 | 63.80 | O |
| ATOM | 8587 | CB | ASP | D | 331 | 65.748 | 17.209 | −52.274 | 1.00 | 90.05 | C |
| ATOM | 8588 | CG | ASP | D | 331 | 66.566 | 18.417 | −52.675 | 1.00 | 99.54 | C |
| ATOM | 8589 | OD1 | ASP | D | 331 | 66.980 | 18.486 | −53.850 | 1.00 | 95.72 | O |
| ATOM | 8590 | OD2 | ASP | D | 331 | 66.795 | 19.295 | −51.814 | 1.00 | 111.50 | O |
| ATOM | 8591 | N | PHE | D | 332 | 63.153 | 15.623 | −52.561 | 1.00 | 71.23 | N |
| ATOM | 8592 | CA | PHE | D | 332 | 62.179 | 14.630 | −52.119 | 1.00 | 69.55 | C |
| ATOM | 8593 | C | PHE | D | 332 | 61.870 | 13.672 | −53.261 | 1.00 | 69.17 | C |
| ATOM | 8594 | O | PHE | D | 332 | 61.815 | 12.457 | −53.073 | 1.00 | 62.25 | O |
| ATOM | 8595 | CB | PHE | D | 332 | 60.885 | 15.301 | −51.648 | 1.00 | 67.26 | C |
| ATOM | 8596 | CG | PHE | D | 332 | 60.948 | 15.846 | −50.244 | 1.00 | 66.82 | C |
| ATOM | 8597 | CD1 | PHE | D | 332 | 61.197 | 17.195 | −50.016 | 1.00 | 62.39 | C |
| ATOM | 8598 | CD2 | PHE | D | 332 | 60.738 | 15.013 | −49.150 | 1.00 | 66.40 | C |
| ATOM | 8599 | CE1 | PHE | D | 332 | 61.242 | 17.710 | −48.719 | 1.00 | 62.29 | C |
| ATOM | 8600 | CE2 | PHE | D | 332 | 60.787 | 15.518 | −47.845 | 1.00 | 60.47 | C |
| ATOM | 8601 | CZ | PHE | D | 332 | 61.040 | 16.871 | −47.633 | 1.00 | 53.48 | C |
| ATOM | 8602 | N | ARG | D | 333 | 61.669 | 14.246 | −54.443 | 1.00 | 71.03 | N |
| ATOM | 8603 | CA | ARG | D | 333 | 61.389 | 13.500 | −55.663 | 1.00 | 59.85 | C |
| ATOM | 8604 | C | ARG | D | 333 | 62.494 | 12.480 | −55.934 | 1.00 | 62.75 | C |
| ATOM | 8605 | O | ARG | D | 333 | 62.228 | 11.281 | −56.028 | 1.00 | 63.64 | O |
| ATOM | 8606 | CB | ARG | D | 333 | 61.283 | 14.480 | −56.827 | 1.00 | 65.60 | C |
| ATOM | 8607 | CG | ARG | D | 333 | 60.158 | 14.222 | −57.797 | 1.00 | 69.61 | C |
| ATOM | 8608 | CD | ARG | D | 333 | 59.833 | 15.501 | −58.563 | 1.00 | 77.89 | C |
| ATOM | 8609 | NE | ARG | D | 333 | 60.998 | 16.384 | −58.677 | 1.00 | 87.50 | N |
| ATOM | 8610 | CZ | ARG | D | 333 | 60.963 | 17.619 | −59.180 | 1.00 | 85.86 | C |
| ATOM | 8611 | NH1 | ARG | D | 333 | 59.820 | 18.124 | −59.624 | 1.00 | 82.44 | N |
| ATOM | 8612 | NH2 | ARG | D | 333 | 62.072 | 18.351 | −59.240 | 1.00 | 64.36 | N |
| ATOM | 8613 | N | LYS | D | 334 | 63.730 | 12.963 | −56.063 | 1.00 | 63.75 | N |
| ATOM | 8614 | CA | LYS | D | 334 | 64.899 | 12.092 | −56.221 | 1.00 | 71.16 | C |
| ATOM | 8615 | C | LYS | D | 334 | 64.877 | 10.979 | −55.181 | 1.00 | 70.64 | C |
| ATOM | 8616 | O | LYS | D | 334 | 65.039 | 9.801 | −55.504 | 1.00 | 62.05 | O |
| ATOM | 8617 | CB | LYS | D | 334 | 66.209 | 12.881 | −56.039 | 1.00 | 87.21 | C |
| ATOM | 8618 | CG | LYS | D | 334 | 66.565 | 13.908 | −57.123 | 1.00 | 87.99 | C |
| ATOM | 8619 | CD | LYS | D | 334 | 67.948 | 14.519 | −56.833 | 1.00 | 88.69 | C |
| ATOM | 8620 | CE | LYS | D | 334 | 68.087 | 15.949 | −57.355 | 1.00 | 88.99 | C |
| ATOM | 8621 | NZ | LYS | D | 334 | 69.167 | 16.716 | −56.646 | 1.00 | 63.98 | N |
| ATOM | 8622 | N | ALA | D | 335 | 64.686 | 11.378 | −53.926 | 1.00 | 73.95 | N |
| ATOM | 8623 | CA | ALA | D | 335 | 64.730 | 10.468 | −52.782 | 1.00 | 71.55 | C |
| ATOM | 8624 | C | ALA | D | 335 | 63.564 | 9.485 | −52.741 | 1.00 | 67.66 | C |
| ATOM | 8625 | O | ALA | D | 335 | 63.741 | 8.333 | −52.358 | 1.00 | 60.79 | O |
| ATOM | 8626 | CB | ALA | D | 335 | 64.801 | 11.256 | −51.482 | 1.00 | 70.69 | C |
| ATOM | 8627 | N | PHE | D | 336 | 62.373 | 9.946 | −53.117 | 1.00 | 70.79 | N |
| ATOM | 8628 | CA | PHE | D | 336 | 61.218 | 9.063 | −53.219 | 1.00 | 72.39 | C |
| ATOM | 8629 | C | PHE | D | 336 | 61.501 | 7.964 | −54.248 | 1.00 | 78.71 | C |
| ATOM | 8630 | O | PHE | D | 336 | 61.080 | 6.821 | −54.081 | 1.00 | 78.71 | O |
| ATOM | 8631 | CB | PHE | D | 336 | 59.952 | 9.830 | −53.631 | 1.00 | 70.56 | C |
| ATOM | 8632 | CG | PHE | D | 336 | 59.462 | 10.826 | −52.606 | 1.00 | 75.48 | C |
| ATOM | 8633 | CD1 | PHE | D | 336 | 59.528 | 10.548 | −51.248 | 1.00 | 76.03 | C |
| ATOM | 8634 | CD2 | PHE | D | 336 | 58.894 | 12.031 | −53.011 | 1.00 | 73.48 | C |
| ATOM | 8635 | CE1 | PHE | D | 336 | 59.063 | 11.466 | −50.308 | 1.00 | 63.21 | C |
| ATOM | 8636 | CE2 | PHE | D | 336 | 58.431 | 12.951 | −52.080 | 1.00 | 69.06 | C |
| ATOM | 8637 | CZ | PHE | D | 336 | 58.519 | 12.667 | −50.725 | 1.00 | 62.63 | C |
| ATOM | 8638 | N | LYS | D | 337 | 62.219 | 8.319 | −55.311 | 1.00 | 79.93 | N |
| ATOM | 8639 | CA | LYS | D | 337 | 62.511 | 7.376 | −56.388 | 1.00 | 81.12 | C |
| ATOM | 8640 | C | LYS | D | 337 | 63.625 | 6.389 | −56.021 | 1.00 | 76.84 | C |
| ATOM | 8641 | O | LYS | D | 337 | 63.493 | 5.189 | −56.265 | 1.00 | 76.58 | O |
| ATOM | 8642 | CB | LYS | D | 337 | 62.823 | 8.115 | −57.696 | 1.00 | 80.16 | C |
| ATOM | 8643 | CG | LYS | D | 337 | 61.691 | 9.024 | −58.164 | 1.00 | 82.54 | C |
| ATOM | 8644 | CD | LYS | D | 337 | 61.806 | 9.380 | −59.647 | 1.00 | 89.62 | C |
| ATOM | 8645 | CE | LYS | D | 337 | 60.631 | 10.249 | −60.100 | 1.00 | 91.61 | C |
| ATOM | 8646 | NZ | LYS | D | 337 | 60.651 | 10.553 | −61.560 | 1.00 | 82.56 | N |
| ATOM | 8647 | N | ARG | D | 338 | 64.716 | 6.886 | −55.443 | 1.00 | 71.06 | N |
| ATOM | 8648 | CA | ARG | D | 338 | 65.725 | 5.996 | −54.874 | 1.00 | 83.59 | C |
| ATOM | 8649 | C | ARG | D | 338 | 65.011 | 4.946 | −54.030 | 1.00 | 83.42 | C |
| ATOM | 8650 | O | ARG | D | 338 | 65.141 | 3.746 | −54.261 | 1.00 | 82.98 | O |
| ATOM | 8651 | CB | ARG | D | 338 | 66.694 | 6.757 | −53.966 | 1.00 | 88.62 | C |
| ATOM | 8652 | CG | ARG | D | 338 | 67.496 | 7.868 | −54.616 | 1.00 | 98.18 | C |
| ATOM | 8653 | CD | ARG | D | 338 | 68.417 | 8.503 | −53.573 | 1.00 | 106.87 | C |
| ATOM | 8654 | NE | ARG | D | 338 | 69.483 | 9.302 | −54.172 | 1.00 | 135.96 | N |
| ATOM | 8655 | CZ | ARG | D | 338 | 70.563 | 9.724 | −53.518 | 1.00 | 134.03 | C |
| ATOM | 8656 | NH1 | ARG | D | 338 | 70.731 | 9.422 | −52.236 | 1.00 | 125.41 | N |
| ATOM | 8657 | NH2 | ARG | D | 338 | 71.480 | 10.448 | −54.149 | 1.00 | 114.84 | N |
| ATOM | 8658 | N | LEU | D | 339 | 64.246 | 5.428 | −53.053 | 1.00 | 88.95 | N |
| ATOM | 8659 | CA | LEU | D | 339 | 63.517 | 4.583 | −52.106 | 1.00 | 87.86 | C |
| ATOM | 8660 | C | LEU | D | 339 | 62.587 | 3.562 | −52.764 | 1.00 | 88.81 | C |
| ATOM | 8661 | O | LEU | D | 339 | 62.336 | 2.491 | −52.207 | 1.00 | 83.89 | O |
| ATOM | 8662 | CB | LEU | D | 339 | 62.692 | 5.456 | −51.157 | 1.00 | 83.67 | C |
| ATOM | 8663 | CG | LEU | D | 339 | 63.398 | 6.362 | −50.147 | 1.00 | 89.49 | C |
| ATOM | 8664 | CD1 | LEU | D | 339 | 62.417 | 7.408 | −49.632 | 1.00 | 82.25 | C |
| ATOM | 8665 | CD2 | LEU | D | 339 | 63.991 | 5.556 | −48.995 | 1.00 | 73.40 | C |
| ATOM | 8666 | N | LEU | D | 340 | 62.057 | 3.898 | −53.934 | 1.00 | 86.32 | N |
| ATOM | 8667 | CA | LEU | D | 340 | 61.143 | 3.000 | −54.628 | 1.00 | 88.02 | C |
| ATOM | 8668 | C | LEU | D | 340 | 61.871 | 2.158 | −55.685 | 1.00 | 85.87 | C |
| ATOM | 8669 | O | LEU | D | 340 | 61.337 | 1.884 | −56.760 | 1.00 | 84.19 | O |
| ATOM | 8670 | CB | LEU | D | 340 | 59.980 | 3.793 | −55.234 | 1.00 | 85.39 | C |
| ATOM | 8671 | CG | LEU | D | 340 | 59.036 | 4.429 | −54.202 | 1.00 | 82.90 | C |
| ATOM | 8672 | CD1 | LEU | D | 340 | 58.348 | 5.686 | −54.736 | 1.00 | 78.73 | C |
| ATOM | 8673 | CD2 | LEU | D | 340 | 58.012 | 3.412 | −53.711 | 1.00 | 72.18 | C |
| ATOM | 8674 | C16 | PDL | D | 400 | 34.229 | 20.502 | −31.029 | 1.00 | 37.57 | C |
| ATOM | 8675 | N3 | PDL | D | 400 | 33.164 | 20.648 | −30.675 | 1.00 | 46.59 | N |
| ATOM | 8676 | N1 | PDL | D | 400 | 36.770 | 20.970 | −30.897 | 1.00 | 37.28 | N |
| ATOM | 8677 | C1 | PDL | D | 400 | 35.570 | 20.327 | −31.476 | 1.00 | 28.68 | C |
| ATOM | 8678 | C2 | PDL | D | 400 | 36.018 | 19.415 | −32.613 | 1.00 | 28.59 | C |
| ATOM | 8679 | C3 | PDL | D | 400 | 37.484 | 19.555 | −32.704 | 1.00 | 30.84 | C |
| ATOM | 8680 | C4 | PDL | D | 400 | 38.459 | 18.903 | −33.655 | 1.00 | 24.90 | C |
| ATOM | 8681 | C5 | PDL | D | 400 | 39.941 | 19.197 | −33.553 | 1.00 | 30.73 | C |
| ATOM | 8682 | C6 | PDL | D | 400 | 40.443 | 20.161 | −32.480 | 1.00 | 34.53 | C |
| ATOM | 8683 | C7 | PDL | D | 400 | 39.462 | 20.833 | −31.522 | 1.00 | 31.87 | C |
| ATOM | 8684 | C8 | PDL | D | 400 | 37.961 | 20.523 | −31.636 | 1.00 | 33.85 | C |
| ATOM | 8685 | O1 | PDL | D | 400 | 37.965 | 18.058 | −34.646 | 1.00 | 35.43 | O |
| ATOM | 8686 | C9 | PDL | D | 400 | 38.363 | 16.721 | −34.734 | 1.00 | 37.29 | C |
| ATOM | 8687 | C10 | PDL | D | 400 | 37.404 | 16.098 | −35.744 | 1.00 | 32.42 | C |
| ATOM | 8688 | O2 | PDL | D | 400 | 38.137 | 15.290 | −36.630 | 1.00 | 27.86 | O |
| ATOM | 8689 | C11 | PDL | D | 400 | 36.335 | 15.315 | −34.963 | 1.00 | 22.61 | C |
| ATOM | 8690 | N2 | PDL | D | 400 | 35.771 | 14.176 | −35.692 | 1.00 | 39.48 | N |
| ATOM | 8691 | C12 | PDL | D | 400 | 34.935 | 13.380 | −34.777 | 1.00 | 41.45 | C |
| ATOM | 8692 | C13 | PDL | D | 400 | 33.676 | 14.227 | −34.453 | 1.00 | 20.04 | C |
| ATOM | 8693 | C14 | PDL | D | 400 | 35.727 | 13.042 | −33.480 | 1.00 | 24.70 | C |
| ATOM | 8694 | C15 | PDL | D | 400 | 34.541 | 12.053 | −35.475 | 1.00 | 28.56 | C |
| ATOM | 8695 | NA | NA | D | 401 | 28.626 | 6.588 | −26.566 | 1.00 | 43.36 | Na |
| ATOM | 8696 | C1 | 8TG | | 500 | 10.993 | 30.178 | 7.889 | 1.00 | 37.04 | C |
| ATOM | 8697 | S1 | 8TG | | 500 | 9.765 | 29.130 | 8.728 | 1.00 | 44.60 | S |
| ATOM | 8698 | C2 | 8TG | | 500 | 11.138 | 29.728 | 6.402 | 1.00 | 37.52 | C |
| ATOM | 8699 | O2 | 8TG | | 500 | 11.795 | 28.486 | 6.285 | 1.00 | 34.58 | O |
| ATOM | 8700 | C3 | 8TG | | 500 | 11.825 | 30.766 | 5.489 | 1.00 | 34.16 | C |
| ATOM | 8701 | O3 | 8TG | | 500 | 11.470 | 30.384 | 4.183 | 1.00 | 38.38 | O |
| ATOM | 8702 | C4 | 8TG | | 500 | 11.275 | 32.160 | 5.791 | 1.00 | 35.70 | C |
| ATOM | 8703 | O4 | 8TG | | 500 | 12.019 | 33.168 | 5.170 | 1.00 | 50.67 | O |
| ATOM | 8704 | C5 | 8TG | | 500 | 11.306 | 32.464 | 7.279 | 1.00 | 44.51 | C |
| ATOM | 8705 | O5 | 8TG | | 500 | 10.500 | 31.537 | 8.035 | 1.00 | 42.28 | O |
| ATOM | 8706 | C6 | 8TG | | 500 | 10.790 | 33.889 | 7.463 | 1.00 | 45.31 | C |
| ATOM | 8707 | O6 | 8TG | | 500 | 9.389 | 33.845 | 7.426 | 1.00 | 47.54 | O |
| ATOM | 8708 | C1′ | 8TG | | 500 | 9.666 | 29.823 | 10.414 | 1.00 | 41.86 | C |
| ATOM | 8709 | C2′ | 8TG | | 500 | 11.085 | 29.848 | 11.032 | 1.00 | 37.52 | C |
| ATOM | 8710 | C3′ | 8TG | | 500 | 11.126 | 28.808 | 12.177 | 1.00 | 39.74 | C |
| ATOM | 8711 | C4′ | 8TG | | 500 | 12.544 | 28.220 | 12.368 | 1.00 | 33.61 | C |
| ATOM | 8712 | C5′ | 8TG | | 500 | 13.597 | 29.354 | 12.407 | 1.00 | 35.15 | C |
| ATOM | 8713 | C6′ | 8TG | | 500 | 14.980 | 28.671 | 12.503 | 1.00 | 45.87 | C |
| ATOM | 8714 | C7′ | 8TG | | 500 | 15.968 | 29.365 | 11.540 | 1.00 | 39.33 | C |
| ATOM | 8715 | C8′ | 8TG | | 500 | 17.398 | 28.888 | 11.867 | 1.00 | 43.93 | C |
| ATOM | 8716 | C1 | 8TG | | 501 | 32.608 | 19.475 | 11.900 | 1.00 | 90.15 | C |
| ATOM | 8717 | S1 | 8TG | | 501 | 31.628 | 17.960 | 11.587 | 1.00 | 93.54 | S |
| ATOM | 8718 | C2 | 8TG | | 501 | 33.626 | 19.696 | 10.750 | 1.00 | 104.82 | C |
| ATOM | 8719 | O2 | 8TG | | 501 | 34.302 | 18.505 | 10.395 | 1.00 | 77.91 | O |
| ATOM | 8720 | C3 | 8TG | | 501 | 34.557 | 20.858 | 11.161 | 1.00 | 115.79 | C |
| ATOM | 8721 | O3 | 8TG | | 501 | 35.467 | 21.085 | 10.115 | 1.00 | 104.95 | O |
| ATOM | 8722 | C4 | 8TG | | 501 | 33.692 | 22.112 | 11.340 | 1.00 | 120.59 | C |
| ATOM | 8723 | O4 | 8TG | | 501 | 34.439 | 23.174 | 11.887 | 1.00 | 114.75 | O |
| ATOM | 8724 | C5 | 8TG | | 501 | 32.447 | 21.890 | 12.218 | 1.00 | 122.15 | C |
| ATOM | 8725 | O5 | 8TG | | 501 | 31.738 | 20.638 | 12.008 | 1.00 | 120.40 | O |
| ATOM | 8726 | C6 | 8TG | | 501 | 31.501 | 23.099 | 12.065 | 1.00 | 109.10 | C |
| ATOM | 8727 | O6 | 8TG | | 501 | 30.488 | 23.093 | 13.037 | 1.00 | 82.15 | O |
| ATOM | 8728 | C1′ | 8TG | | 501 | 30.222 | 18.330 | 10.469 | 1.00 | 53.70 | C |
| ATOM | 8729 | C2′ | 8TG | | 501 | 29.563 | 16.996 | 10.048 | 1.00 | 39.87 | C |
| ATOM | 8730 | C3′ | 8TG | | 501 | 28.320 | 16.751 | 10.935 | 1.00 | 34.30 | C |
| ATOM | 8731 | C4′ | 8TG | | 501 | 27.208 | 16.163 | 10.052 | 1.00 | 22.54 | C |
| ATOM | 8732 | C5′ | 8TG | | 501 | 26.674 | 14.878 | 10.731 | 1.00 | 16.34 | C |
| ATOM | 8733 | C6′ | 8TG | | 501 | 25.235 | 14.679 | 10.209 | 1.00 | 18.79 | C |
| ATOM | 8734 | C7′ | 8TG | | 501 | 25.041 | 13.222 | 9.743 | 1.00 | 26.21 | C |
| ATOM | 8735 | C8′ | 8TG | | 501 | 23.603 | 13.076 | 9.237 | 1.00 | 18.47 | C |
| ATOM | 8736 | C1 | 8TG | | 502 | 31.062 | 22.948 | −17.988 | 1.00 | 63.93 | C |
| ATOM | 8737 | S1 | 8TG | | 502 | 31.491 | 24.487 | −18.897 | 1.00 | 88.98 | S |
| ATOM | 8738 | C2 | 8TG | | 502 | 29.787 | 22.257 | −18.566 | 1.00 | 72.41 | C |
| ATOM | 8739 | O2 | 8TG | | 502 | 28.709 | 23.169 | −18.623 | 1.00 | 77.66 | O |
| ATOM | 8740 | C3 | 8TG | | 502 | 29.445 | 20.982 | −17.747 | 1.00 | 68.42 | C |
| ATOM | 8741 | O3 | 8TG | | 502 | 28.305 | 20.342 | −18.288 | 1.00 | 51.95 | O |
| ATOM | 8742 | C4 | 8TG | | 502 | 30.667 | 20.050 | −17.795 | 1.00 | 55.05 | C |
| ATOM | 8743 | O4 | 8TG | | 502 | 30.474 | 18.850 | −17.077 | 1.00 | 41.32 | O |
| ATOM | 8744 | C5 | 8TG | | 502 | 31.968 | 20.754 | −17.349 | 1.00 | 56.59 | C |
| ATOM | 8745 | O5 | 8TG | | 502 | 32.214 | 22.054 | −17.951 | 1.00 | 56.64 | O |
| ATOM | 8746 | C6 | 8TG | | 502 | 32.105 | 20.825 | −15.807 | 1.00 | 75.21 | C |
| ATOM | 8747 | O6 | 8TG | | 502 | 33.129 | 21.699 | −15.377 | 1.00 | 60.34 | O |
| ATOM | 8748 | C1′ | 8TG | | 502 | 33.304 | 24.586 | −19.169 | 1.00 | 42.60 | C |
| ATOM | 8749 | C2′ | 8TG | | 502 | 33.686 | 23.994 | −20.546 | 1.00 | 27.43 | C |
| ATOM | 8750 | C3′ | 8TG | | 502 | 35.148 | 23.481 | −20.495 | 1.00 | 30.02 | C |
| ATOM | 8751 | C4′ | 8TG | | 502 | 36.133 | 24.513 | −21.102 | 1.00 | 32.30 | C |
| ATOM | 8752 | C5′ | 8TG | | 502 | 36.762 | 23.924 | −22.397 | 1.00 | 35.98 | C |
| ATOM | 8753 | C6′ | 8TG | | 502 | 38.306 | 24.074 | −22.368 | 1.00 | 32.25 | C |
| ATOM | 8754 | C7′ | 8TG | | 502 | 38.901 | 23.416 | −23.643 | 1.00 | 27.76 | C |
| ATOM | 8755 | C8′ | 8TG | | 502 | 40.179 | 24.166 | −24.074 | 1.00 | 27.49 | C |
| ATOM | 8756 | C1 | 8TG | | 503 | 38.078 | 7.692 | −18.116 | 1.00 | 34.01 | C |
| ATOM | 8757 | S1 | 8TG | | 503 | 37.381 | 8.321 | −19.659 | 1.00 | 48.31 | S |
| ATOM | 8758 | C2 | 8TG | | 503 | 38.737 | 6.287 | −18.184 | 1.00 | 32.37 | C |
| ATOM | 8759 | O2 | 8TG | | 503 | 37.807 | 5.367 | −18.721 | 1.00 | 38.95 | O |
| ATOM | 8760 | C3 | 8TG | | 503 | 39.064 | 5.877 | −16.731 | 1.00 | 33.15 | C |
| ATOM | 8761 | O3 | 8TG | | 503 | 39.978 | 4.818 | −16.777 | 1.00 | 53.58 | O |
| ATOM | 8762 | C4 | 8TG | | 503 | 39.729 | 7.001 | −15.940 | 1.00 | 32.23 | C |
| ATOM | 8763 | O4 | 8TG | | 503 | 39.631 | 6.718 | −14.569 | 1.00 | 65.01 | O |
| ATOM | 8764 | C5 | 8TG | | 503 | 39.186 | 8.411 | −16.195 | 1.00 | 36.99 | C |
| ATOM | 8765 | O5 | 8TG | | 503 | 38.948 | 8.702 | −17.586 | 1.00 | 32.95 | O |
| ATOM | 8766 | C6 | 8TG | | 503 | 40.181 | 9.470 | −15.738 | 1.00 | 47.42 | C |
| ATOM | 8767 | O6 | 8TG | | 503 | 40.173 | 10.410 | −16.778 | 1.00 | 37.05 | O |
| ATOM | 8768 | C1′ | 8TG | | 503 | 38.121 | 9.963 | −19.919 | 1.00 | 36.29 | C |
| ATOM | 8769 | C2′ | 8TG | | 503 | 38.495 | 10.094 | −21.418 | 1.00 | 51.31 | C |
| ATOM | 8770 | C3′ | 8TG | | 503 | 39.174 | 8.801 | −21.946 | 1.00 | 29.03 | C |
| ATOM | 8771 | C4′ | 8TG | | 503 | 39.815 | 9.077 | −23.330 | 1.00 | 27.01 | C |
| ATOM | 8772 | C5′ | 8TG | | 503 | 41.159 | 9.842 | −23.184 | 1.00 | 24.46 | C |
| ATOM | 8773 | C6′ | 8TG | | 503 | 42.327 | 8.909 | −22.767 | 1.00 | 27.21 | C |
| ATOM | 8774 | C7′ | 8TG | | 503 | 43.657 | 9.561 | −23.229 | 1.00 | 48.34 | C |
| ATOM | 8775 | C8′ | 8TG | | 503 | 44.820 | 9.277 | −22.259 | 1.00 | 28.64 | C |
| ATOM | 8776 | C1 | 8TG | | 504 | 23.266 | 27.271 | −22.442 | 1.00 | 106.91 | C |
| ATOM | 8777 | S1 | 8TG | | 504 | 24.230 | 28.721 | −23.001 | 1.00 | 88.64 | S |
| ATOM | 8778 | C2 | 8TG | | 504 | 21.769 | 27.515 | −22.773 | 1.00 | 116.38 | C |
| ATOM | 8779 | O2 | 8TG | | 504 | 21.344 | 28.799 | −22.371 | 1.00 | 105.09 | O |
| ATOM | 8780 | C3 | 8TG | | 504 | 20.983 | 26.422 | −22.037 | 1.00 | 124.77 | C |
| ATOM | 8781 | O3 | 8TG | | 504 | 19.595 | 26.661 | −22.121 | 1.00 | 97.72 | O |
| ATOM | 8782 | C4 | 8TG | | 504 | 21.374 | 25.071 | −22.644 | 1.00 | 125.35 | C |
| ATOM | 8783 | O4 | 8TG | | 504 | 20.732 | 24.920 | −23.884 | 1.00 | 117.30 | O |
| ATOM | 8784 | C5 | 8TG | | 504 | 22.900 | 24.856 | −22.811 | 1.00 | 124.17 | C |
| ATOM | 8785 | O5 | 8TG | | 504 | 23.743 | 26.027 | −23.028 | 1.00 | 107.69 | O |
| ATOM | 8786 | C6 | 8TG | | 504 | 23.182 | 23.798 | −23.903 | 1.00 | 114.67 | C |
| ATOM | 8787 | O6 | 8TG | | 504 | 24.551 | 23.473 | −23.991 | 1.00 | 72.69 | O |
| ATOM | 8788 | C1′ | 8TG | | 504 | 25.758 | 28.308 | −23.916 | 1.00 | 30.56 | C |
| ATOM | 8789 | C2′ | 8TG | | 504 | 26.428 | 29.663 | −24.256 | 1.00 | 32.55 | C |
| ATOM | 8790 | C3′ | 8TG | | 504 | 27.880 | 29.428 | −24.741 | 1.00 | 42.76 | C |
| ATOM | 8791 | C4′ | 8TG | | 504 | 28.874 | 30.473 | −24.171 | 1.00 | 30.69 | C |
| ATOM | 8792 | C5′ | 8TG | | 504 | 30.225 | 30.284 | −24.926 | 1.00 | 42.38 | C |
| ATOM | 8793 | C6′ | 8TG | | 504 | 31.484 | 30.595 | −24.071 | 1.00 | 27.05 | C |
| ATOM | 8794 | C7′ | 8TG | | 504 | 32.749 | 30.447 | −24.958 | 1.00 | 29.84 | C |
| ATOM | 8795 | C8′ | 8TG | | 504 | 33.956 | 31.242 | −24.409 | 1.00 | 22.84 | C |
| ATOM | 8796 | C1 | 8TG | | 505 | 3.430 | 16.859 | 6.928 | 1.00 | 66.25 | C |
| ATOM | 8797 | S1 | 8TG | | 505 | 2.975 | 15.077 | 6.894 | 1.00 | 110.67 | S |
| ATOM | 8798 | C2 | 8TG | | 505 | 4.034 | 17.386 | 5.566 | 1.00 | 72.15 | C |
| ATOM | 8799 | O2 | 8TG | | 505 | 4.001 | 16.400 | 4.549 | 1.00 | 98.40 | O |
| ATOM | 8800 | C3 | 8TG | | 505 | 3.566 | 18.793 | 5.038 | 1.00 | 87.45 | C |
| ATOM | 8801 | O3 | 8TG | | 505 | 3.308 | 18.793 | 3.647 | 1.00 | 85.83 | O |
| ATOM | 8802 | C4 | 8TG | | 505 | 2.400 | 19.453 | 5.778 | 1.00 | 81.01 | C |
| ATOM | 8803 | O4 | 8TG | | 505 | 2.543 | 20.858 | 5.837 | 1.00 | 63.58 | O |
| ATOM | 8804 | C5 | 8TG | | 505 | 2.428 | 18.975 | 7.211 | 1.00 | 64.81 | C |
| ATOM | 8805 | O5 | 8TG | | 505 | 2.237 | 17.564 | 7.368 | 1.00 | 71.69 | O |
| ATOM | 8806 | C6 | 8TG | | 505 | 1.490 | 19.823 | 8.058 | 1.00 | 60.70 | C |
| ATOM | 8807 | O6 | 8TG | | 505 | 2.287 | 20.324 | 9.097 | 1.00 | 68.65 | O |
| ATOM | 8808 | C1′ | 8TG | | 505 | 3.906 | 14.159 | 8.175 | 1.00 | 60.99 | C |
| ATOM | 8809 | C2′ | 8TG | | 505 | 5.365 | 14.683 | 8.258 | 1.00 | 45.47 | C |
| ATOM | 8810 | C3′ | 8TG | | 505 | 5.896 | 14.352 | 9.675 | 1.00 | 41.77 | C |
| ATOM | 8811 | C4′ | 8TG | | 505 | 7.352 | 14.825 | 9.892 | 1.00 | 37.65 | C |
| ATOM | 8812 | C5′ | 8TG | | 505 | 7.999 | 13.908 | 10.967 | 1.00 | 29.55 | C |
| ATOM | 8813 | C6′ | 8TG | | 505 | 9.506 | 14.200 | 11.127 | 1.00 | 38.75 | C |
| ATOM | 8814 | C7′ | 8TG | | 505 | 9.713 | 15.109 | 12.363 | 1.00 | 45.04 | C |
| ATOM | 8815 | C8′ | 8TG | | 505 | 11.192 | 15.051 | 12.796 | 1.00 | 25.19 | C |
| ATOM | 8816 | C1′ | 8TG | | 506 | −2.430 | 10.405 | 12.432 | 1.00 | 27.63 | C |
| ATOM | 8817 | C2′ | 8TG | | 506 | −2.097 | 8.958 | 12.878 | 1.00 | 31.79 | C |
| ATOM | 8818 | C3′ | 8TG | | 506 | −0.758 | 8.868 | 13.664 | 1.00 | 33.40 | C |
| ATOM | 8819 | C4′ | 8TG | | 506 | 0.430 | 8.460 | 12.756 | 1.00 | 41.94 | C |
| ATOM | 8820 | C5′ | 8TG | | 506 | 1.611 | 7.945 | 13.632 | 1.00 | 44.21 | C |
| ATOM | 8821 | C6′ | 8TG | | 506 | 2.984 | 8.042 | 12.905 | 1.00 | 38.42 | C |
| ATOM | 8822 | C7′ | 8TG | | 506 | 4.126 | 7.789 | 13.928 | 1.00 | 39.56 | C |
| ATOM | 8823 | C8′ | 8TG | | 506 | 5.513 | 7.582 | 13.276 | 1.00 | 28.31 | C |
| ATOM | 8824 | C1 | 8TG | | 507 | 28.439 | 3.060 | 41.727 | 1.00 | 86.98 | C |
| ATOM | 8825 | S1 | 8TG | | 507 | 28.607 | 3.947 | 40.129 | 1.00 | 104.80 | S |
| ATOM | 8826 | C2 | 8TG | | 507 | 29.828 | 2.439 | 42.050 | 1.00 | 87.16 | C |
| ATOM | 8827 | O2 | 8TG | | 507 | 30.146 | 1.485 | 41.068 | 1.00 | 94.81 | O |
| ATOM | 8828 | C3 | 8TG | | 507 | 29.868 | 1.780 | 43.438 | 1.00 | 83.65 | C |
| ATOM | 8829 | O3 | 8TG | | 507 | 31.201 | 1.444 | 43.718 | 1.00 | 80.40 | O |
| ATOM | 8830 | C4 | 8TG | | 507 | 29.456 | 2.830 | 44.455 | 1.00 | 102.43 | C |
| ATOM | 8831 | O4 | 8TG | | 507 | 29.460 | 2.255 | 45.739 | 1.00 | 85.58 | O |
| ATOM | 8832 | C5 | 8TG | | 507 | 28.074 | 3.398 | 44.109 | 1.00 | 107.02 | C |
| ATOM | 8833 | O5 | 8TG | | 507 | 27.982 | 3.964 | 42.776 | 1.00 | 94.18 | O |
| ATOM | 8834 | C6 | 8TG | | 507 | 27.628 | 4.420 | 45.168 | 1.00 | 99.65 | C |
| ATOM | 8835 | O6 | 8TG | | 507 | 26.229 | 4.532 | 45.157 | 1.00 | 93.29 | O |
| ATOM | 8836 | C1′ | 8TG | | 507 | 27.143 | 4.985 | 39.754 | 1.00 | 82.08 | C |
| ATOM | 8837 | C2′ | 8TG | | 507 | 27.499 | 5.991 | 38.628 | 1.00 | 68.13 | C |
| ATOM | 8838 | C3′ | 8TG | | 507 | 27.289 | 5.372 | 37.223 | 1.00 | 52.73 | C |
| ATOM | 8839 | C4′ | 8TG | | 507 | 26.044 | 6.020 | 36.579 | 1.00 | 38.44 | C |
| ATOM | 8840 | C5′ | 8TG | | 507 | 26.270 | 6.213 | 35.063 | 1.00 | 39.71 | C |
| ATOM | 8841 | C6′ | 8TG | | 507 | 27.146 | 7.465 | 34.838 | 1.00 | 51.90 | C |
| ATOM | 8842 | C7′ | 8TG | | 507 | 28.020 | 7.272 | 33.581 | 1.00 | 36.63 | C |
| ATOM | 8843 | C8′ | 8TG | | 507 | 27.150 | 6.726 | 32.430 | 1.00 | 60.74 | C |
| ATOM | 8844 | C1 | DMU | | 510 | 62.736 | 16.453 | −22.348 | 1.00 | 116.44 | C |
| ATOM | 8845 | C10 | DMU | | 510 | 66.802 | 15.892 | −20.286 | 1.00 | 141.54 | C |
| ATOM | 8846 | C11 | DMU | | 510 | 69.835 | 17.900 | −21.184 | 1.00 | 116.62 | C |
| ATOM | 8847 | C18 | DMU | | 510 | 60.720 | 19.342 | −22.513 | 1.00 | 68.47 | C |
| ATOM | 8848 | C19 | DMU | | 510 | 59.249 | 19.715 | −22.750 | 1.00 | 44.14 | C |
| ATOM | 8849 | C2 | DMU | | 510 | 63.836 | 15.811 | −21.476 | 1.00 | 130.58 | C |
| ATOM | 8850 | C22 | DMU | | 510 | 58.684 | 20.283 | −21.432 | 1.00 | 44.98 | C |
| ATOM | 8851 | C25 | DMU | | 510 | 57.148 | 20.435 | −21.479 | 1.00 | 35.88 | C |
| ATOM | 8852 | C28 | DMU | | 510 | 56.772 | 21.801 | −20.872 | 1.00 | 29.61 | C |
| ATOM | 8853 | C3 | DMU | | 510 | 64.672 | 16.872 | −20.718 | 1.00 | 137.39 | C |
| ATOM | 8854 | C31 | DMU | | 510 | 55.791 | 22.583 | −21.768 | 1.00 | 23.05 | C |
| ATOM | 8855 | C34 | DMU | | 510 | 54.389 | 22.609 | −21.127 | 1.00 | 19.25 | C |
| ATOM | 8856 | C37 | DMU | | 510 | 53.753 | 24.004 | −21.288 | 1.00 | 15.93 | C |
| ATOM | 8857 | C4 | DMU | | 510 | 63.751 | 17.907 | −20.014 | 1.00 | 130.14 | C |
| ATOM | 8858 | C40 | DMU | | 510 | 52.472 | 24.084 | −20.428 | 1.00 | 22.57 | C |
| ATOM | 8859 | C43 | DMU | | 510 | 51.851 | 25.487 | −20.508 | 1.00 | 23.10 | C |
| ATOM | 8860 | C5 | DMU | | 510 | 67.395 | 14.647 | −19.567 | 1.00 | 153.52 | C |
| ATOM | 8861 | C57 | DMU | | 510 | 63.240 | 17.385 | −18.650 | 1.00 | 106.97 | C |
| ATOM | 8862 | C6 | DMU | | 510 | 61.877 | 17.425 | −21.500 | 1.00 | 111.64 | C |
| ATOM | 8863 | C7 | DMU | | 510 | 68.943 | 14.511 | −19.570 | 1.00 | 149.84 | C |
| ATOM | 8864 | C8 | DMU | | 510 | 69.702 | 15.843 | −19.743 | 1.00 | 142.90 | C |
| ATOM | 8865 | C9 | DMU | | 510 | 69.011 | 16.638 | −20.867 | 1.00 | 138.50 | C |
| ATOM | 8866 | O1 | DMU | | 510 | 67.683 | 17.019 | −20.468 | 1.00 | 145.50 | O |
| ATOM | 8867 | O16 | DMU | | 510 | 60.811 | 17.963 | −22.257 | 1.00 | 93.04 | O |
| ATOM | 8868 | O2 | DMU | | 510 | 69.551 | 16.577 | −18.548 | 1.00 | 126.18 | O |
| ATOM | 8869 | O3 | DMU | | 510 | 66.893 | 13.510 | −20.227 | 1.00 | 153.38 | O |
| ATOM | 8870 | O4 | DMU | | 510 | 69.335 | 13.597 | −20.567 | 1.00 | 129.69 | O |
| ATOM | 8871 | O49 | DMU | | 510 | 61.937 | 15.430 | −22.901 | 1.00 | 103.89 | O |
| ATOM | 8872 | O5 | DMU | | 510 | 62.653 | 18.436 | −20.806 | 1.00 | 100.03 | O |
| ATOM | 8873 | O55 | DMU | | 510 | 64.629 | 14.971 | −22.282 | 1.00 | 126.79 | O |
| ATOM | 8874 | O6 | DMU | | 510 | 69.294 | 18.527 | −22.317 | 1.00 | 118.67 | O |
| ATOM | 8875 | O61 | DMU | | 510 | 62.012 | 17.983 | −18.323 | 1.00 | 77.40 | O |
| ATOM | 8876 | O7 | DMU | | 510 | 65.525 | 16.240 | −19.765 | 1.00 | 135.87 | O |
| ATOM | 8877 | C18 | DMU | | 511 | 18.925 | 9.748 | 16.297 | 1.00 | 44.00 | C |
| ATOM | 8878 | C19 | DMU | | 511 | 17.534 | 9.105 | 16.106 | 1.00 | 51.16 | C |
| ATOM | 8879 | C22 | DMU | | 511 | 17.162 | 8.980 | 14.608 | 1.00 | 41.98 | C |
| ATOM | 8880 | C25 | DMU | | 511 | 17.198 | 7.509 | 14.140 | 1.00 | 33.39 | C |
| ATOM | 8881 | C28 | DMU | | 511 | 16.027 | 7.193 | 13.177 | 1.00 | 48.72 | C |
| ATOM | 8882 | C31 | DMU | | 511 | 14.840 | 6.556 | 13.940 | 1.00 | 51.48 | C |
| ATOM | 8883 | C34 | DMU | | 511 | 14.051 | 5.549 | 13.073 | 1.00 | 43.12 | C |
| ATOM | 8884 | C37 | DMU | | 511 | 13.010 | 4.817 | 13.945 | 1.00 | 47.34 | C |
| ATOM | 8885 | C40 | DMU | | 511 | 11.824 | 4.325 | 13.091 | 1.00 | 38.48 | C |
| ATOM | 8886 | C43 | DMU | | 511 | 10.501 | 4.627 | 13.819 | 1.00 | 28.99 | C |
| ATOM | 8887 | O | HOH | | 2 | 2.783 | 31.451 | 14.797 | 1.00 | 54.63 | O |
| ATOM | 8888 | O | HOH | | 6 | 30.179 | 7.916 | −24.831 | 1.00 | 41.19 | O |
| ATOM | 8889 | O | HOH | | 7 | 7.986 | 38.338 | 14.733 | 1.00 | 32.37 | O |
| ATOM | 8890 | O | HOH | | 8 | 42.716 | −0.857 | 25.658 | 1.00 | 47.72 | O |
| ATOM | 8891 | O | HOH | | 9 | 41.521 | −3.114 | 28.145 | 1.00 | 59.36 | O |
| ATOM | 8892 | O | HOH | | 10 | 11.506 | 19.784 | 15.060 | 1.00 | 13.55 | O |
| ATOM | 8893 | O | HOH | | 11 | 7.044 | 26.934 | 11.072 | 1.00 | 20.10 | O |
| ATOM | 8894 | O | HOH | | 12 | −6.971 | 8.262 | 17.962 | 1.00 | 38.26 | O |
| ATOM | 8895 | O | HOH | | 13 | 8.363 | 17.264 | 32.381 | 1.00 | 26.48 | O |
| ATOM | 8896 | O | HOH | | 14 | 30.921 | 13.599 | −15.216 | 1.00 | 20.42 | O |
| ATOM | 8897 | O | HOH | | 15 | 29.552 | 9.877 | −4.325 | 1.00 | 21.82 | O |
| ATOM | 8898 | O | HOH | | 16 | 37.679 | 13.512 | 8.617 | 1.00 | 28.28 | O |
| ATOM | 8899 | O | HOH | | 17 | 39.661 | −0.205 | 5.263 | 1.00 | 60.94 | O |
| ATOM | 8900 | O | HOH | | 18 | 17.910 | −3.424 | 0.360 | 1.00 | 46.24 | O |
| ATOM | 8901 | O | HOH | | 19 | 57.365 | 31.834 | 4.236 | 1.00 | 26.03 | O |
| ATOM | 8902 | O | HOH | | 20 | 58.636 | 24.845 | 4.503 | 1.00 | 25.09 | O |
| ATOM | 8903 | O | HOH | | 21 | 57.610 | 27.774 | −6.483 | 1.00 | 31.23 | O |
| ATOM | 8904 | O | HOH | | 22 | 62.365 | 30.490 | −1.467 | 1.00 | 32.34 | O |
| ATOM | 8905 | O | HOH | | 23 | 45.990 | 41.409 | −11.157 | 1.00 | 39.81 | O |
| ATOM | 8906 | O | HOH | | 24 | 36.124 | 0.372 | −25.587 | 1.00 | 23.54 | O |
| ATOM | 8907 | O | HOH | | 25 | 34.998 | 11.750 | −21.896 | 1.00 | 28.39 | O |
| ATOM | 8908 | O | HOH | | 26 | 22.086 | 5.561 | −17.348 | 1.00 | 30.52 | O |
| ATOM | 8909 | O | HOH | | 27 | 32.502 | 10.968 | −29.158 | 1.00 | 41.93 | O |
| ATOM | 8910 | O | HOH | | 28 | 39.530 | 18.364 | −25.865 | 1.00 | 17.99 | O |
| ATOM | 8911 | O | HOH | | 29 | 36.707 | 20.814 | −43.464 | 1.00 | 21.15 | O |
| ATOM | 8912 | O | HOH | | 30 | 43.882 | 15.460 | −41.512 | 1.00 | 30.63 | O |
| ATOM | 8913 | O | HOH | | 31 | 54.025 | 16.325 | −43.651 | 1.00 | 43.31 | O |

1. A method comprising:
providing the coordinates of the turkey β1-AR structure listed in Table A, Table B, Table C or Table D, optionally varied by a root mean square deviation of residue backbone atoms of not more than 1.235 Å, or selected coordinates thereof.

2. A method according to claim 1 further comprising predicting the three-dimensional structural representation of a target protein of unknown structure, or part thereof, by modelling the structural representation on all of the selected coordinates of the turkey β1-AR; and
optionally aligning the amino acid sequence of the target protein of unknown structure with the amino acid sequence of turkey β1-AR listed in FIG. 7 to match homologous regions of the amino acid sequences prior to predicting the structural representation, and wherein modeling the structural representation comprises modeling the structural representation of the matched homologous regions of the target protein on the corresponding regions of the β1-AR to obtain a three dimensional structural representation for the target protein that substantially preserves the structural representation of the matched homologous regions.

3. A method of claim 1 further comprising
either (a) positioning the coordinates in the crystal unit cell of a target protein of unknown structure, or part thereof, so as to predict its structural representation, or (b) assigning NMR spectra peaks of the protein by manipulating the coordinates.

4. A method of claim 1 further comprising
providing an X-ray diffraction pattern of the target protein; and
using the coordinates to predict at least part of the structure coordinates of the target protein.

5.-8. (canceled)

9. A method of claim 1, further comprising using molecular modelling means to select or design one or more binding partners of β1-AR, wherein the three-dimensional structural representation of at least part of turkey β1-AR, as defined by the coordinates of turkey β1-AR listed in Table A, Table B, Table C or Table D, optionally varied by a root mean square deviation of residue backbone atoms of not more than 1.235 Å, or selected coordinates thereof, is compared with a three-dimensional structural representation of one or more candidate binding partners, and one or more binding partners that are predicted to interact with β1-AR are selected,
optionally wherein the three-dimensional structural representation of the one or more candidate binding partners is obtained by: providing structural representations of a plurality of molecular fragments; fitting the structural representation of each of the molecular fragments to the coordinates of the turkey β1-AR listed in Table A, Table B, Table C or Table D, optionally varied by a root mean square deviation of residue backbone atoms of not more than 1.235 Å, or selected coordinates thereof; and assembling the representations of the molecular fragments into one or more representations of single molecules to provide the three-dimensional structural representation of one or more candidate binding partners.

10. A method of claim 1 further comprising analyzing the interaction of one or more binding partners with β1-AR by a method comprising:
providing a three dimensional structural representation of one or more binding partners to be fitted to the structural representation of β1-AR or selected coordinates thereof; and
fitting the one of more binding partners to said structure.

11.-14. (canceled)

15. A method according to claim 9, further comprising the steps of:
obtaining or synthesising the one or more binding partners; and either:
(I) contacting the one or more binding partners with a β1-AR to determine the ability of the one or more binding partners to interact with the β1-AR; or
(II) forming one or more complexes of a β1-AR and a binding partner and analysing the one or more complexes by X-ray crystallography to determine the ability of the one or more binding partners to interact with β1-AR; or
(III) forming one or more crystallised complexes of a β1-AR and a binding partner and analysing the one or more complexes by X-ray crystallography by employing the coordinates of the turkey β1-AR structure, listed in Table A, Table B, Table C or Table D, optionally varied by a root mean square deviation of residue backbone atoms of not more than 1.235 Å, or selected coordinates thereof, to determine the ability of the one or more binding partners to interact with the β1-AR,
optionally wherein the one or more crystallised complexes are formed by either (a) providing a crystal of β1-AR and soaking the crystal with the binding partner to form a complex; or (b) mixing β1-AR with the binding partner and crystallising a β1-AR-binding partner complex.

16.-18. (canceled)

19. A method for producing a binding partner of β1-AR comprising:
identifying a binding partner according to the method of claim 9, and synthesising the binding partner.

20. A binding partner produced by the method of claim 19, optionally wherein the binding partner is a full agonist, a partial agonist, an inverse agonist or an antagonist of β1-AR.

21. A method of claim 1 further comprising:
providing an X-ray diffraction pattern of β1-AR complexed with a β1-AR binding partner, or part thereof, which binds to β1-AR; and
using said coordinates to predict at least part of the structure coordinates of the binding partner,
optionally wherein the X-ray diffraction pattern is from a crystal formed either by (a) soaking a crystal of β1-AR with the binding partner to form a complex, or (b) mixing β1-AR with the binding partner and crystallising a β1-AR-binding partner complex,
thereby predicting the three dimensional structure of a binding partner of unknown structure, or part thereof, which binds to β1-AR.

22.-26. (canceled)

27. A pharmaceutical composition comprising the binding partner according to claim 20.

28. A method of providing data for generating three dimensional structural representations of β1-AR, β1-AR homologues or analogues, complexes of β1-AR with binding partners, or complexes of β1-AR homologues or analogues with binding partners, or, for analysing or optimising binding of binding partners to said β1-AR or homologues or analogues, or complexes thereof, the method comprising:
(i) establishing communication with a remote device containing computer-readable data comprising at least one of:
(a) the coordinates of the turkey β1-AR structure provided in claim 1;
(b) the coordinates of a target β1-AR homologue or analogue generated by homology modelling of the target based on the data in (a);
(c) the coordinates of a binding partner generated by interpreting X-ray crystallographic data or NMR data by reference to the coordinates of the turkey β1-AR structure and
(d) structure factor data derivable from the coordinates of (a), (b) or (c); and
(ii) receiving said computer-readable data from said remote device.

29. A method of claim 1 further comprising generating a three-dimensional structural representation of said coordinates,
optionally wherein the three-dimensional structural representation is a computer generated representation or a physical representation,
optionally wherein the computer used to generate the representation comprises:
(i) a computer-readable data storage medium comprising a data storage material encoded with computer-readable data, wherein said data comprise the coordinates of the turkey β1-AR structure, listed in Table A, Table B, Table C or Table D, optionally varied by a root mean square deviation of residue backbone atoms of not more than 1.235 Å, or selected coordinates thereof; and
(ii) instructions for processing the computer-readable data into a three-dimensional structural representation.

30.-32. (canceled)

33. A method of claim 1 further comprising:
analysing said coordinates to predict one or more sites of interaction; or
analysing said coordinates to predict the location of internal and/or external parts of the structure; or
performing a statistical and/or a topological analysis on the coordinates; and comparing the results of the analysis with the results of an analysis of coordinates of proteins of known activation states.

34.-37. (canceled)

38. A computer system, intended to generate three dimensional structural representations of β1-AR, β1-AR homologues or analogues, complexes of β1-AR with binding partners, or complexes of β1-AR homologues or analogues with binding partners, or, to analyse or optimise binding of binding partners to said β1-AR or homologues or analogues, or complexes thereof, the system containing computer-readable data comprising one or more of:
(a) the coordinates of the turkey β1-AR structure provided in claim 1;
(b) the coordinates of a target β1-AR homologue or analogue generated by homology modelling of the target based on the data in (a);
(c) the coordinates of a binding partner generated by interpreting X-ray crystallographic data or NMR data by reference to the coordinates of the turkey β1-AR structure, listed in Table A, Table B, Table C or Table D, optionally varied by a root mean square deviation of residue backbone atoms of not more than 1.235 Å, or selected coordinates thereof, and
(d) structure factor data derivable from the coordinates of (a), (b) or (c).

39. A computer system according to claim 38, comprising:
(i) a computer-readable data storage medium comprising data storage material encoded with the computer-readable data;
(ii) a working memory for storing instructions for processing the computer-readable data; and
(iii) a central processing unit coupled to the working memory and to the computer-readable data storage medium for processing the computer-readable data to generate said structural representations or to analyse or optimise said binding; and
optionally comprising a display coupled to the central-processing unit for displaying structural representations.

40. (canceled)

41. A computer-readable storage medium, comprising a data storage material encoded with
(I) computer readable data, wherein the data comprises one or more of
(a) the coordinates of the turkey β1-AR structure provided in claim 1;
(b) the coordinates of a target β1-AR homologue or analogue generated by homology modelling of the target based on the data in (a);
(c) the coordinates of a binding partner generated by interpreting X-ray crystallographic data or NMR data by reference to the coordinates of the turkey β1-AR structure, listed in Table A, Table B, Table C or Table D, optionally varied by a root mean square deviation of residue backbone atoms of not more than 1.235 Å, or selected coordinates thereof, and
(d) structure factor data derivable from the coordinates of (a), (b) or (c); or
(II) a first set of computer-readable data comprising a Fourier transform of at least a portion of the structural coordinates of turkey β1-AR provided in claim 1; which data, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data.

42.-44. (canceled)

45. A method of producing a protein with a binding region that has substrate specificity substantially identical to that of β1-AR, the method comprising
a) aligning the amino acid sequence of a target protein with the amino acid sequence of a β1-AR;
b) identifying the amino acid residues in the target protein that correspond to any one or more of the following positions according to the numbering of the turkey β1-AR, as set out in (SEQ ID NO:4), 117, 118, 121, 122, 125, 201, 203, 207, 211, 215, 306, 307, 310 and 329; and
c) making one or more mutations in the amino acid sequence of the target protein to replace one or more identified amino acid residues with the corresponding residue in the turkey β1-AR.

46. A peptide of not more than 100 amino acid residues in length comprising at least five contiguous amino acid residues which define an external structural moiety of the β1-AR.

47. (canceled)

48. A mutant β1-AR, wherein the β1-AR before mutation has a binding region in the position equivalent to the binding region of turkey β1-AR that is defined by residues including 117, 118, 121, 122, 125, 201, 203, 207, 211, 215, 306, 307, 310 and 329 of β1-AR and wherein one or more residues equivalent to 117, 118, 121, 122, 125, 201, 203, 207, 211, 215, 306, 307, 310 and 329 forming part of the binding region of β1-AR is mutated.

49. A method of making a β1-AR crystal comprising:
providing purified β1-AR; and
crystallising the β1-AR either by using the sitting drop or hanging drop vapour diffusion technique, using a precipitant solution comprising 0.1M ADA (N-(2-acetaimido) immunodiacetic acid) (pH5.6-9.5) and 25-35% PEG 600, optionally wherein the precipitant solution comprises 0.1M ADA (pH 6.9-7.3) and 29-32% PEG600.

50. (canceled)

51. A crystal of β1-AR having the structure defined by the coordinates of the β1-AR structure provided in claim 1.

52.-56. (canceled)
